(19) United States
(12) Patent Application Publication
(10) Pub. No.: US 2025/0332266 A1
(43) Pub. Date: Oct. 30, 2025

(54) COMPOUNDS FOR THE DEGRADATION OF EGFR KINASE

(71) Applicant: BeiGene Switzerland GmbH, Basel (CH)

(72) Inventors: Bailin LEI, Beijing (CN); Huaqing LIU, Beijing (CN); Yizhou ZHAO, Beijing (CN); Songzhe HAN, Beijing (CN); Wei LIU, Beijing (CN); Xinzhu QI, Beijing, (CN); Zhiwei WANG, Beijing, (CN)

(73) Assignee: BeiGene Switzerland GmbH, Basel (CH)

(21) Appl. No.: 19/204,126

(22) Filed: May 9, 2025

Related U.S. Application Data
(63) Continuation of application No. PCT/CN2023/130774, filed on Nov. 9, 2023.

(30) Foreign Application Priority Data
Nov. 10, 2022 (WO) PCT/CN2022/131085
May 31, 2023 (WO) PCT/CN2023/097329

(51) Int. Cl. A61K 47/54; C07D 498/22

(52) U.S. Cl. CPC A61K 47/545; C07D 498/22

(57) ABSTRACT

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis targeting chimera (PROTAC) consists of two covalently linked protein-binding molecules: one capable of engaging an E3 ubiquitin ligase, and another that binds to the protein of interest (POI) a target meant for degradation (Sakamoto K M et al., Proc. Natl. Acad. Sci. 2001, 98: 8554-9.; Sakamoto K. M. et al., Methods Enzymol. 2005; 399:833-847.). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (cIAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4A^{CRBN} E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347.; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018; 61: 6685-6704. Winter G E. et al., Science 2015; 348:1376-1381.) In the recent years, PROTACs as useful modulators promote the selective degradation of a wide range of target proteins have been reported in antitumor studies. (Lu J. et al., Chem Biol. 2015; 22(6):755-763; Ottis P. et al., Chem Biol. 2017; 12(4):892-898.; Crews C. M. et al., J Med Chem. 2018; 61(2):403-404; Neklesa T. K. et al., Pharmacol Ther. 2017, 174:138-144.; Cermakova K. et al., Molecules, 2018.23(8).; An S. et al., EBioMedicine, 2018.; Lebraud H. et al., Essays Biochem. 2017; 61(5): 517-527.; Sun Y. H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110(2019); Shaodong Liu et al., Medicinal Chemistry Research, volume 29, pages 802-808(2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, WO2018071606, WO2021178920, WO2021127283, WO2021127190, WO202111871 and WO202111913.

Epidermal growth factor receptor (EGFR) that belongs to the ErbB family is a transmembrane receptor tyrosine kinase (RTK), which plays a fundamentally key role in cell proliferation, differentiation, and motility (Y. Yarden, et al., Nat. Rev. Mol. Cell Biol. 2001; 2:127-137.). Homo- or heterodimerization of EGFR and other ErbB family members activates cytoplasmic tyrosine kinase domains to initiate intracellular signaling. Overexpression or activating mutations of EGFR are associated the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707.). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611.). The first-generation EGFR tyrosine kinase inhibitors (EGFR-TKIs) gefitinib and erlotinib have approved for NSCLC patients with EGFR activation mutations (M. Maemondo, N. Engl. J. Med. 362 (2010) 2380-2388.). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), is also called “gatekeeper” T790M mutation (Xu Y., et al. Cancer Biol Ther. 2010, 9 (8): 572-582.). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562.). There is a need for drugs which can overcome EGFR (C797S) resistance obstacle in non-small cell lung cancer (NSCLC). EGFR-Targeting PROTACs serve as a potential strategy to overcome drug resistance mediated by these mutants, which has been disclosed or discussed in patent publications, e.g. WO2018119441, WO2019149922, WO2019183523, WO2019121562, US20190106417, WO202157882, WO2021123087, WO2021133809, WO2021168074, WO2021208918 and WO2021216440.

Although, a number of EGFR-targeting PROTACs which were designed to degrade EGFR mutant proteins have been published (Zhang X., et al. Eur. J. Med. Chem. 2020, 192, 112199.; Zhang H, et al. Eur. J. Med. Chem. 2020, 189, 112061.; Lu X, Med. Res. Rev. 2018, 38(5):1550-1581. He K., et al. Bioorg. Med. Chem. Lett. 2020, 15, 127167.). Most of the published molecules are based on first, second, and third generation of EGFR inhibitors (WO2021023233, WO2019121562 and WO2018119441) or allosteric EGFR inhibitors (WO2021127561). However, there were no data which showed those EGFR-Targeting PROTACs degrading all the main EGFR mutations, Such as Del19, L858R, Del19/T790M, L858R/T790M, Del19/T790M/C797S, L858R/T790M/C797S.

The present application provides novel bifunctional compounds and compositions for the treatment of serious diseases.

### SUMMARY OF THE INVENTION

One objective of the present invention is to provide compounds and derivatives formed by conjugating EGFR inhibitor moieties with E3 ligase Ligand moieties, which function to recruit targeted proteins to E3 ubiquitin ligase for degradation, and methods of preparation and uses thereof.

The compounds described herein or salts thereof are useful in the treatment of a disease that can be affected by EGFR modulation. The present invention provides the use of the compounds described herein or a pharmaceutically acceptable salt thereof in the manufacture of a medicament for the treatment of a disease that can be affected by EGFR modulation. The present invention further provides a compound described herein or a pharmaceutically acceptable salt thereof, for use in the treatment of a disease that can be affected by EGFR modulation. The present application further provides a method of treating a proliferative disorder, comprising administering to a subject in need thereof a therapeutically effective amount of the compounds described herein or a pharmaceutically acceptable salt thereof.

Aspect 1. A compound of Formula (I):

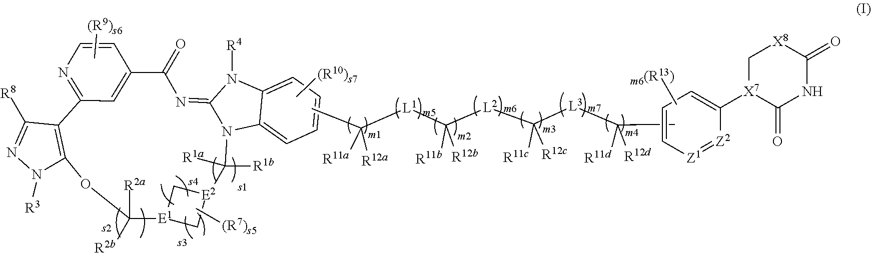

or a N-oxide thereof, or a pharmaceutically acceptable salt thereof, or a stereoisomer thereof, or a tautomer thereof, or a deuterated analog thereof, or a prodrug thereof,
wherein:
E¹ is N or CR⁵;
E² is N or CR⁶;
R^{1a}, R^{1b}, R^{2a} and R^{2b} are each independently absence, hydrogen, halogen, —C₁₋₈alkyl, —C₂₋₈alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈ alkoxy, or —C₃₋₈cycloalkyl is optionally substituted with at least one substituent selected from hydrogen, halogen, —C₁₋₈alkoxy, —C₃₋₈cycloalkyl or —CN;
R³ and R⁴ are each independently hydrogen, —C₁₋₆alkyl, or —C₃₋₈cycloalkyl; each said —C₁₋₆alkyl or —C₃₋₈cycloalkyl is optionally substituted with at least one substituent selected from hydrogen, halogen, —C₁₋₆alkoxy;
R⁵ and R⁶ are each independently absence, hydrogen, halogen, —C₁₋₈alkyl, —C₂₋₈alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl or —CN; or
R⁵ and R⁶ with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;
R⁷ is each independently absence, hydrogen, halogen, —C₁₋₈alkyl, —C₂₋₈alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl or —CN; or
two R⁷ with the carbon atom(s) to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;
R⁸ and R⁹ are each independently selected from hydrogen, halogen, —C₁-C₆alkyl or C₃-C₈cycloalkyl; each of —C₁-C₆alkyl or C₃-C₈cycloalkyl is optionally substituted with at least one substituent selected from hydrogen, halogen, —C₁₋₆alkoxy;
R¹⁰ is each independently selected from hydrogen, halogen, —C₁-C₈alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b} or —NR^{10a}SO₂R^{10b} or —CN; each of —C₁-C₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl is optionally substituted with at least one R^{10c};
R^{10a} and R^{10b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{10d};
R^{10c} and R^{10d} are each independently selected from halogen, hydrogen, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₂aryl, 5- to 12-membered heteroaryl, oxo (═O), —NR^{10c}R^{10f}, —OR^{10c}, —SR^{10c}, —SO₂R^{10c}, —SO₂NR^{10c}R^{10f}, —C(O)R^{10c}, —CO₂R^{10c}, —C(O)NR^{10c}R^{10f}, —NR^{10c}COR^{10f}, —NR^{10c}CO₂R^{8f} or —NR^{10c}SO₂R^{10f} or —CN;
R^{10e} and R^{10f} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R^{11a}, R^{11b}, R^{11c}, R^{11d}, R^{12a}, R^{12b}, R^{12c} and R^{12d} are each independently absence, oxo, hydrogen, halogen, —C₁₋₈alkyl, —C₂₋₈alkenyl, —C₂₋₈alkynyl, —C₁₋₈alkoxy or —C₃₋₈cycloalkyl; each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy or —CN;
L¹ is independently selected from —O—, —NR^{a}—, —C(O)—, *^{L1}-C(O)NR^{a}—**^{L1}, *^{L1}-C(O)O—**^{L1}, *^{L1}-NR^{a}C(O)—**^{L1}, *^{L1}-OC(O)—**^{L1},

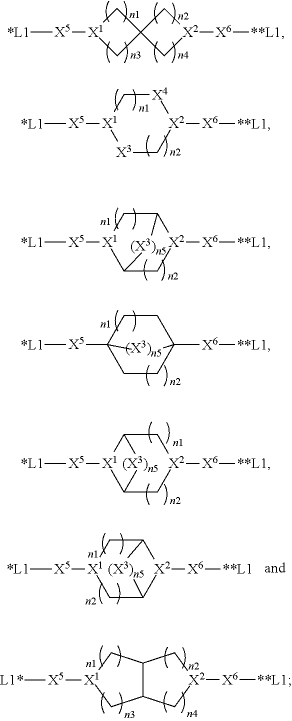

wherein each of said

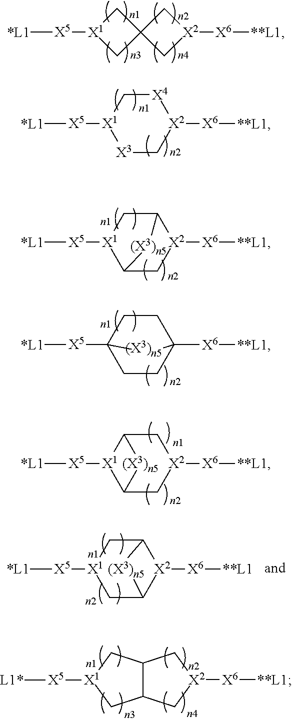

is optionally substituted with at least one R^{L1c};
wherein *^{L1} refers to the position attached to the

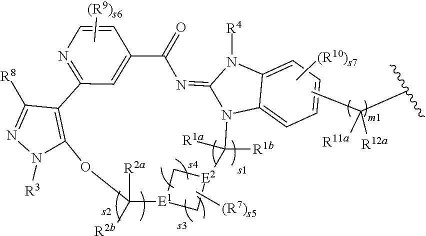

moiety, and **^{L1} refers to the position attached to the

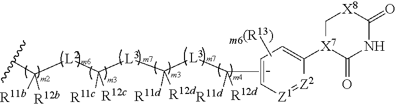

moiety
L² is independently selected from —O—, —NR^{a}—, —C(O)—, *^{L2}-C(O)NR^{a}—**^{L2}, *^{L2}-C(O)O—**^{L2}, *^{L2}-NR^{a}C(O)—**^{L2}, *^{L2}-OC(O)—**^{L2},

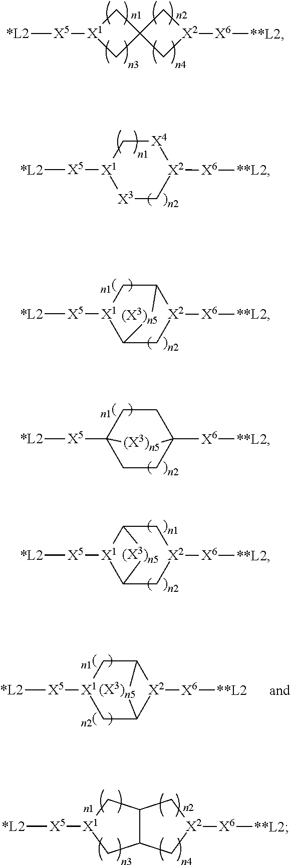

wherein each of said

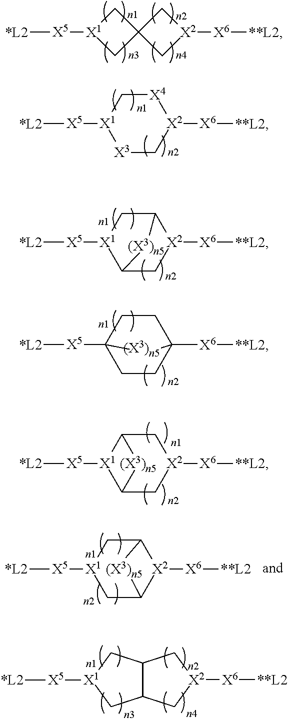

is optionally substituted with at least one R^{L2c};
wherein *^{L2} refers to the position attached to the

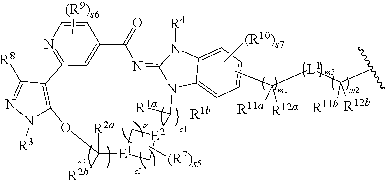

moiety, and **^{L2} refers to the position attached to the

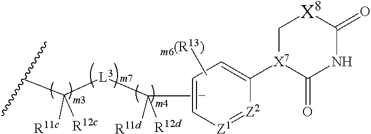

moiety;
L³ is independently selected from —O—, —NR^{a}—, —C(O)—, *^{L3}-C(O)NR^{a}-**^{L3}, *^{L3}-C(O)O—**^{L3}, *^{L3}-NR^{a}C(O)—**^{L3}, *^{L3}-C(O)—**^{L3},

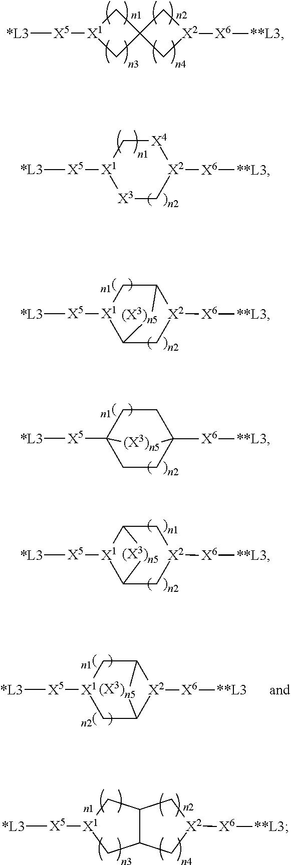

wherein each of said

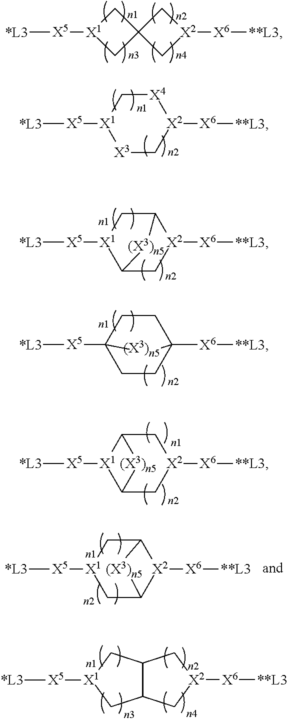

is optionally substituted with at least one R^{L3c};
wherein *^{L3} refers to the position attached to the

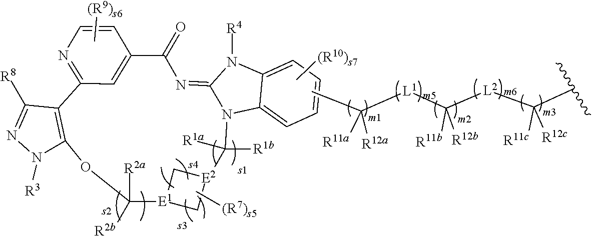

moiety, and **^{L3} refers to the position attached to the

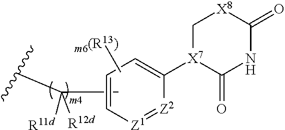

moiety;
each of said R^{L1c}, R^{L2c} and R^{L3c} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
Z¹ and Z² are each independently N or CR^{z};
R^{z}, at each occurrence, is independently selected from absence, hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
R^{Za} and R^{Zb} are each independently selected from absence, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ is each independently selected from absence, hydrogen, halogen, —C₁₋₈alkyl, —C₂₋₈alkenyl, —C₂-C₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{3a}CO₂R^{13b}, or —NR^{13a}SO₂R^{13b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absence, hydrogen, —C₁₋₈alkyl, —C₂-C₈alkenyl, —C₂₋₈alkynyl, C₃-C₈cycloalkyl, 3- to 8-membered heterocyclyl, C₆-C₂aryl, or 5- to 12-membered heteroaryl;
at each occurrence, X¹, X² and X⁷ are each independently selected from —CR^{a}, or N;
at each occurrence, X³, X⁴ and X⁸ are each independently selected from —NR^{a}—, —O—, —S— and —CR^{a}R^{b}—;
at each occurrence, X⁵ and X⁶ are each independently selected from absence, single bond, —C(O)—, —NR^{a}— and —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
m1, m2, m3 and m4 are each independently 0, 1 or 2; provided that m1+m2+m3+m4≤4;
m5, m6 and m7 are each independently 0, 1 or 2; provided that m5+m6+m7≥1;
n1, n2, n3, n4 and n5 are each independently 0, 1, 2 or 3;
n6 is each independently 0, 1, 2, 3 or 4;
s1 and s2 are each independently 0, 1, 2 or 3;
s3 and s4 are each independently 1, 2 or 3;
s5, s6 and s7 are each independently 0, 1, 2 or 3;
provided that:
for any one of L¹, L² or L³, when X¹ is N, X⁵ is single bond, absence, —C(O)—; and/or when X² is N, X⁶ is single bond, absence, —C(O)—;
when L¹ is

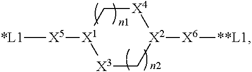

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L² is

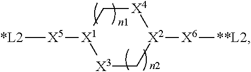

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L³ is

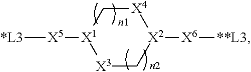

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—.

Aspect 2. The compound of Aspect 1, wherein the compound is selected from formula (IIa) or (IIb),

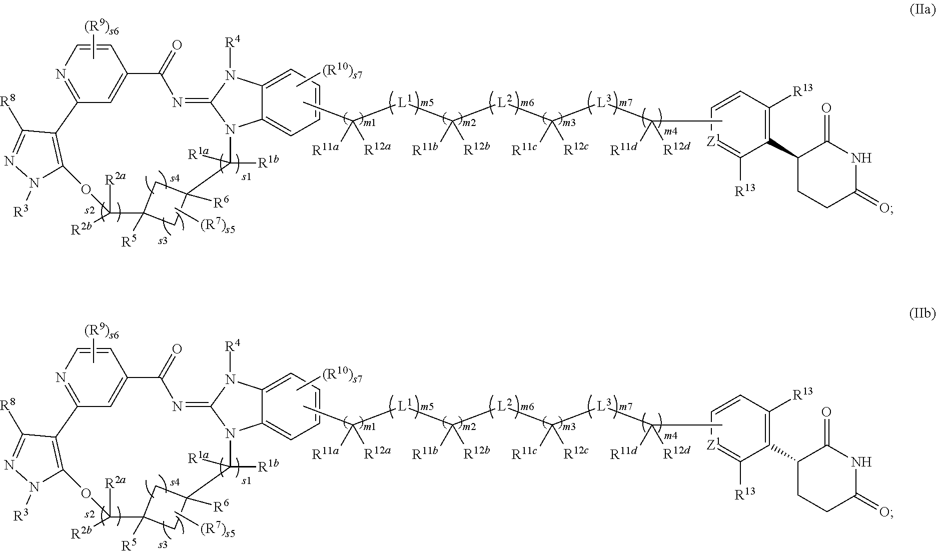

wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12a}, R¹³, L¹, L², L³, Z¹, s1, s2, s3, s4, s5, s6, s7, m1, m2, m3, m4, m5, m6 and m7 are as defined in any one of the preceding Aspects;
more preferably, the compound is selected from formula (IIIa) or (IIIb),

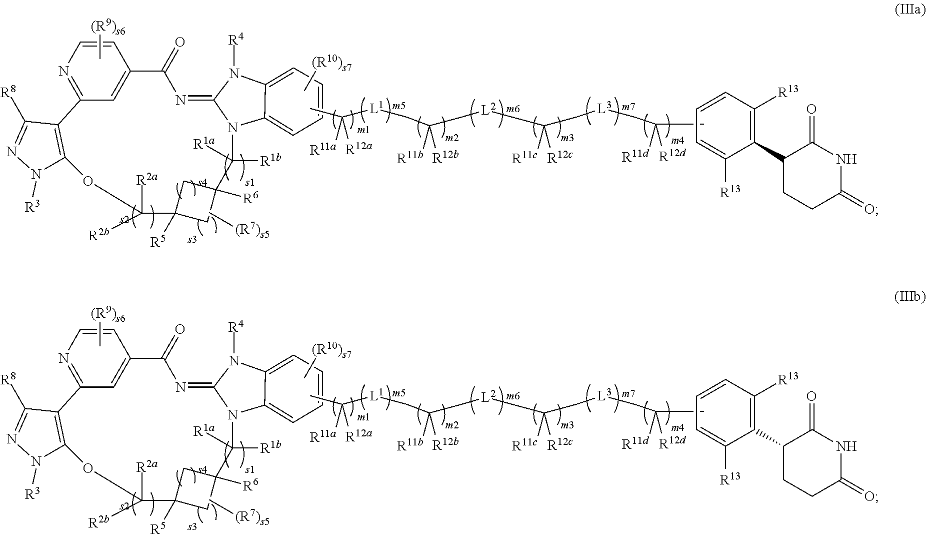

wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12a}, R¹³, L¹, L², L³, s1, s2, s3, s4, s5, s6, s7, m1, m2, m3, m4, m5, m6 and m7 are as defined in any one of the preceding Aspects;
even more preferably, the compound is selected from formula (IVa) or (IVb),

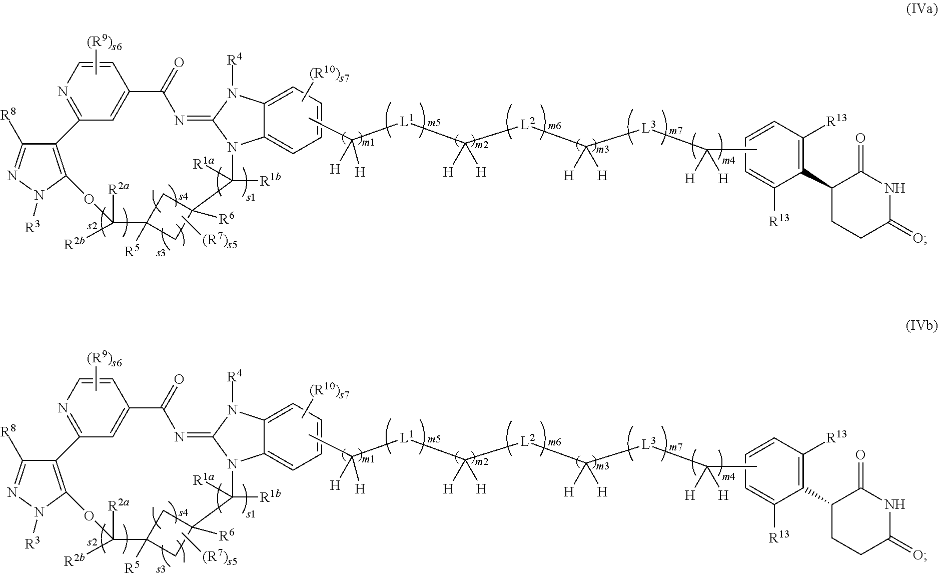

wherein, R^{1a}, R^{1b}, R^{2a}, R^{2b}, R³, R⁴, R⁵, R⁶, R⁷, R⁸, R⁹, R¹⁰, R¹³, L¹, L², L³, s1, s2, s3, s4, s5, s6, s7, m1, m2, m3, m4, m5, m6 and m7 are as defined in any one of the preceding Aspects;
even more preferably, the compound is selected from formula (Va) or (Vb),

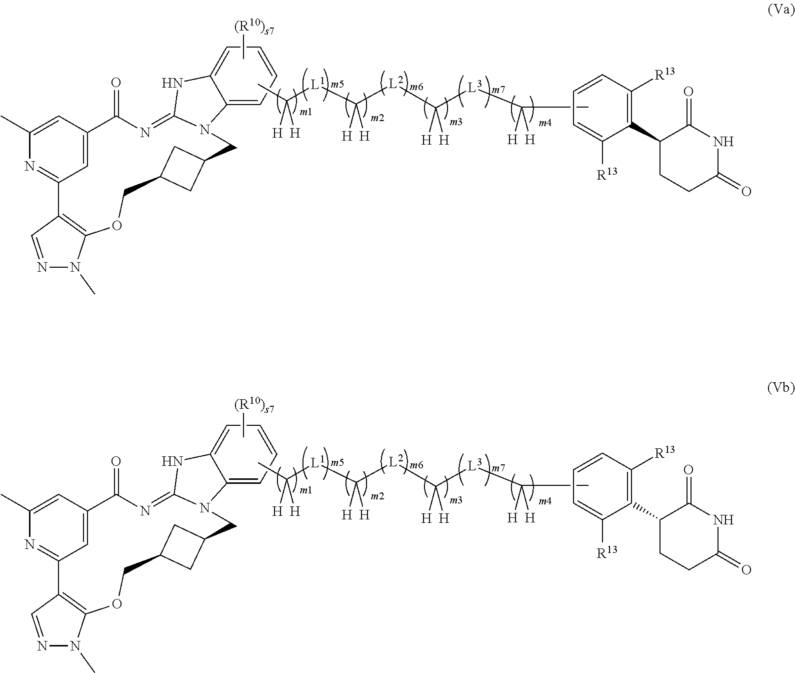

wherein, R¹⁰, R¹³, L¹, L², L³, s7, m1, m2, m3, m4, m5, m6 and m7 are as defined in any one of the preceding Aspects.

Aspect 3. The compound of any one of the preceding Aspects, wherein the compound is selected from formula (VIa),

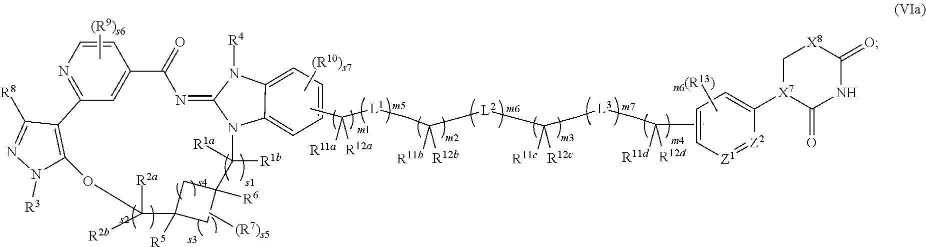

preferably, the compound is selected from formula (VIb), (VIc) or (VIc′)

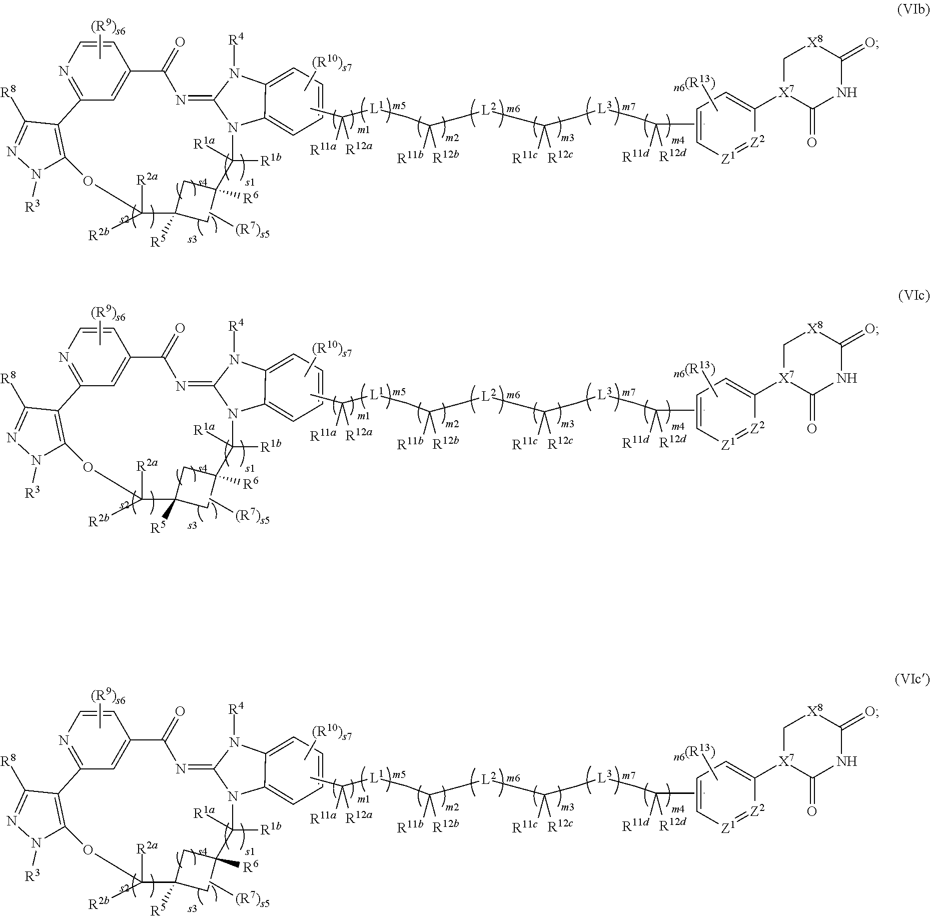

more preferably, the compound is selected from formula (VId) or (VIe)

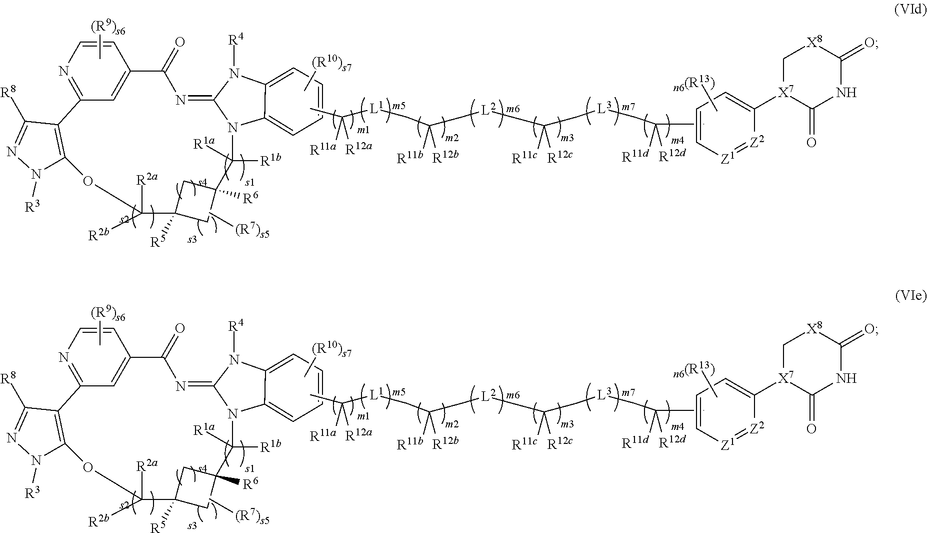

more preferably, the compound is selected from formula (VIf) or (VIg)

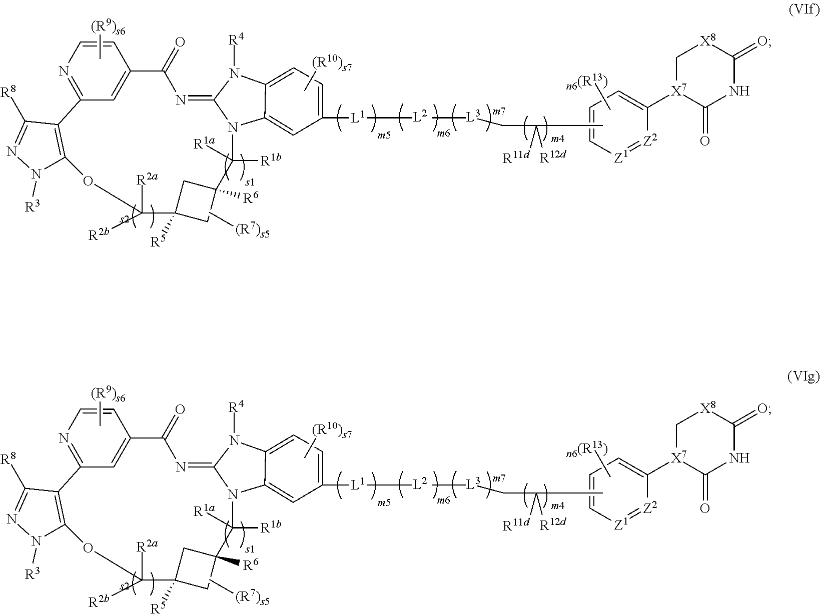

even more preferably, the compound is selected from formula (VIh) or (VIi)

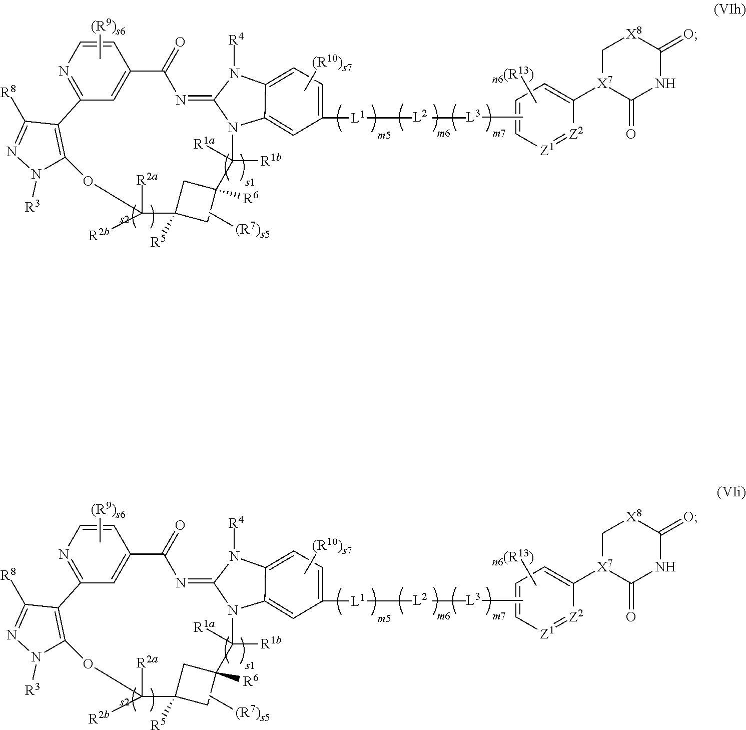

even more preferably, the compound is selected from formula (VIj), (VIk), (VIl) or (VIm)

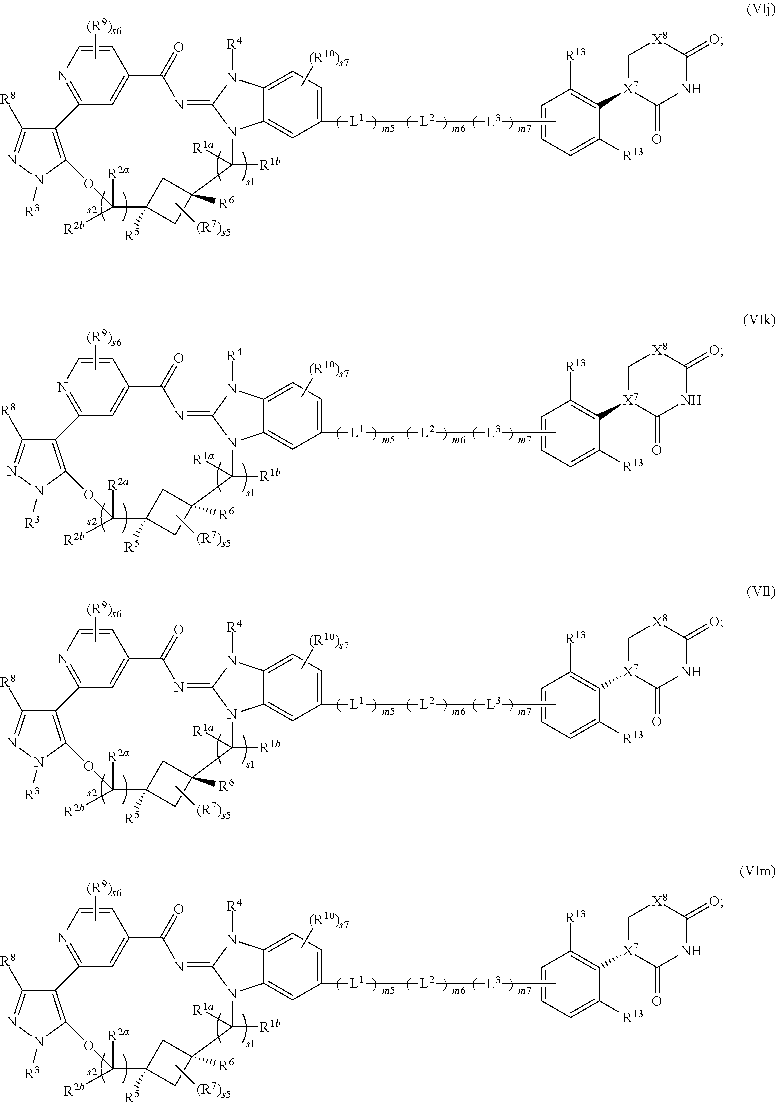

even more preferably, the compound is selected from formula (VIn), (VIo), (VIp) or (VIq)

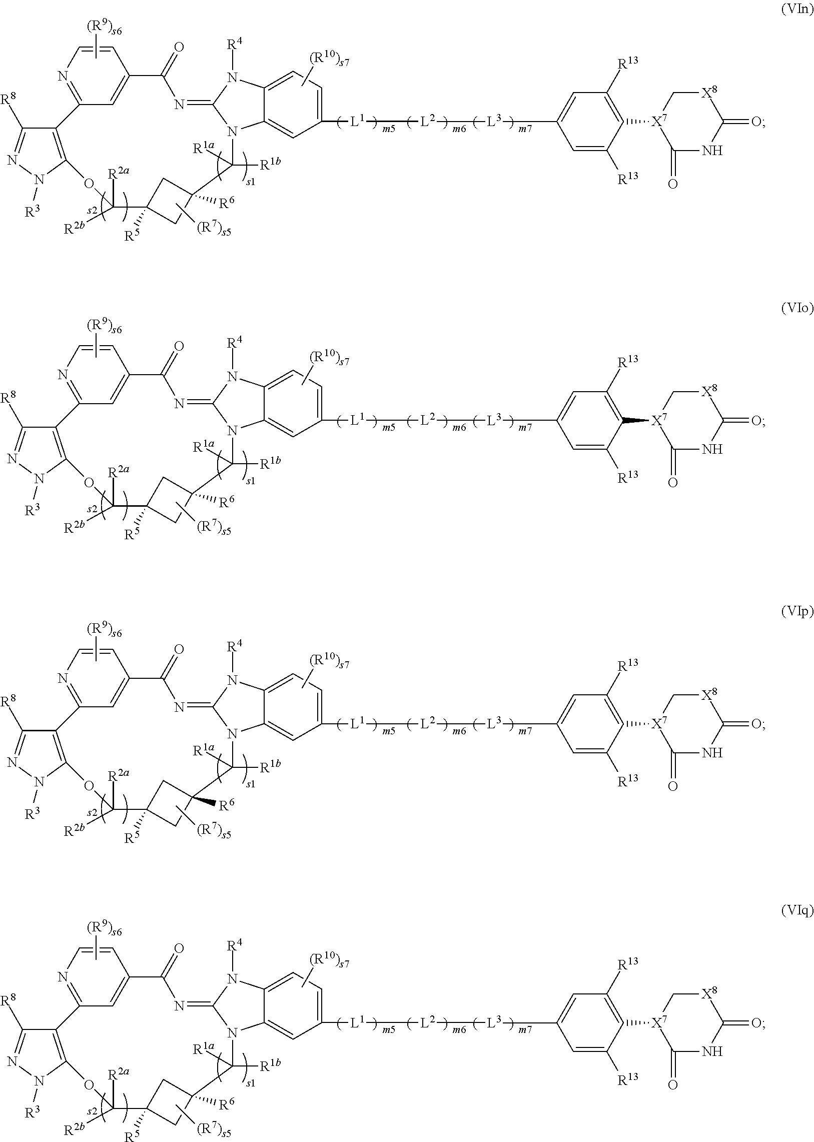

wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{12a}, R^{12b}, R^{12c}, R^{12c}, R¹³, L¹, L², L³, s1, s2, s3, s4, s5, s6, s7, m1, m2, m3, m4, m5, m6, m7, n6, Z¹, Z², X⁷ and X⁸ are as defined in any one of the preceding Aspects.

Aspect 4. The compound of any one of the preceding Aspects, wherein m1+m2+m3+m4≤3.

Aspect 5. The compound of any one of the preceding Aspects, wherein m1+m2+m3+m4=0, 1, 2 or 3; preferably, m1+m2+m3+m4=0, 1 or 2.

Aspect 6. The compound of any one of the preceding Aspects, wherein the number of —CH₂— in

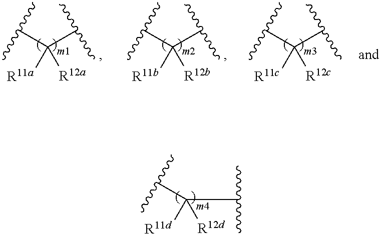

moieties is no more than 4, preferably is no more than 3, even more preferably is no more than 2.

Aspect 7. The compound of any one of the preceding Aspects, wherein R³ and R⁴ are each independently hydrogen, methyl, ethyl, propyl, butyl, pentyl, hexyl, cyclopropyl, cyclobutyl, cyclopentyl, cyclohexyl, cycloheptyl or cyclooctyl; said methyl, ethyl, propyl, butyl, pentyl, hexyl, cyclopropyl, cyclobutyl, cyclopentyl, cyclohexyl, cycloheptyl or cyclooctyl is optionally substituted with at least one substituent selected from hydrogen, F, Cl, Br, I, methoxy, ethoxy, propoxy, butoxy, pentoxy or hexoxy;
preferably, R³ and R⁴ are each independently hydrogen, methyl, ethyl, propyl, butyl, pentyl, hexyl, heptyl, cyclobutyl, cyclopentyl, cyclohexyl, cycloheptyl or cyclooctyl; preferably R³ is independently methyl, and R⁴ is hydrogen.

Aspect 8. The compound of any one of the preceding Aspects, wherein R^{1a}, R^{1b}, R^{2a} and R^{2b} are each independently hydrogen, F, Cl, Br, T, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN; wherein each said methyl, ethyl, propyl, butyl, pentyl, hexyl, heptyl, octyl, —C₂-salkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl and cyclooctyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN.

Aspect 9. The compound of any one of the preceding Aspects, wherein R^{1a}, R^{1b}, R^{2a} and R^{2b} are each independently hydrogen, F, Cl, Br, T, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, —CH₂CH₂OCH₂CH₃;
preferably R^{1a}, R^{1b}, R^{2a} and R^{2b} are each independently hydrogen, F, Cl, methyl, methoxy, cyclopropyl, —CF₃ or —CHF₂, —CH₂OCH₃.

Aspect 10. The compound of any one of the preceding Aspects, wherein R⁵ and R⁶ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, —CH₂CH₂OCH₂CH₃;
preferably R⁵ and R⁶ are each independently hydrogen, F, C₁, methyl, methoxy, cyclopropyl, —CF₃ or —CHF₂, —CH₂OCH₃.

Aspect 11. The compound of any one of the preceding Aspects, wherein R⁵ and R^{e} with the carbon atoms to which they are attached, form a 3-, 4-, 5-, 6-, 7- or 8-membered ring, said ring comprising 0-3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl or octyl;
preferably, R⁵ and R⁶ with the carbon atoms to which they are attached, form a 3-, 4-, 5- or 6-membered ring, said ring is optionally substituted with at least one substituent F, Cl, Br, I, hydroxy, methyl, ethyl, propyl, butyl, pentyl, hexyl, heptyl or octyl.

Aspect 12. The compound of any one of the preceding Aspects, wherein R⁷ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, —CH₂CH₂OCH₂CH₃;
preferably R⁷ are each independently hydrogen, F, Cl, methyl, methoxy, cyclopropyl, —CF₃ or —CHF₂, —CH₂OCH₃.

Aspect 13. The compound of any one of the preceding Aspects, wherein two R⁷ with the carbon atom(s) to which they are attached, form a 3-, 4-, 5-, 6-, 7- or 8-membered ring, said ring comprising 0-3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl or octyl;
preferably, two R⁷ with the carbon atom(s) to which they are attached, form a 3-, 4-, 5- or 6-membered ring, said ring is optionally substituted with at least one substituent F, Cl, Br, I, hydroxy, methyl, ethyl, propyl, butyl, pentyl, hexyl, heptyl or octyl.

Aspect 14. The compound of any one of the preceding Aspects, wherein R⁸ and R⁹ are each independently selected from hydrogen, F, Cl, Br, I, methyl, ethyl, propyl, butyl, pentyl, hexyl, cyclopropyl, cyclobutyl, cyclopentyl, cyclohexyl, cycloheptyl or cyclooctyl; said methyl, ethyl, propyl, butyl, pentyl, hexyl, cyclopropyl, cyclobutyl, cyclopentyl, cyclohexyl, cycloheptyl or cyclooctyl is optionally substituted with at least one substituent selected from hydrogen, F, Cl, Br, I, methoxy, ethoxy, propoxy, butoxy, pentoxy or hexoxy;
preferably, R⁸ and R⁹ are each independently hydrogen, F, Cl, Br, I, methyl, ethyl, propyl, butyl, pentyl, hexyl, heptyl, cyclobutyl, cyclopentyl, cyclohexyl, cycloheptyl or cyclooctyl; preferably R⁸ is independently hydrogen, and R⁹ is F or methyl.

Aspect 15. The compound of any one of the preceding Aspects, wherein R¹⁰ is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b} or —NR^{10a}SO₂R^{10b} or —CN; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl is optionally substituted with at least one R^{10c};
R^{10a} and R^{10b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl, each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl is optionally substituted with at least one substituent R^{10d};
R^{10c} and R^{10d} are each independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{10e}R^{10f}, —OR^{10e}, —SR^{10e}, —SO₂R^{10e}, —SO₂NR^{10e}R^{10f}, —C(O)R^{10e}, —CO₂R^{10e}, —C(O)NR^{10e}R^{10f}, —NR^{10e}COR^{10f}, —NR^{10e}CO₂R^{10f} or —NR^{10e}SO₂R^{10f} or —CN;
R^{10e} and R^{10f} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, R¹⁰ is each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b} or —NR^{10a}SO₂R^{10b} or —CN; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl is optionally substituted with at least one R^{10c}
R^{10a} and R^{10b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl;
R^{10c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{10e}R^{10f}, —OR^{10e} or —CN;
R^{10e} and R^{10f} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R¹⁰ is each independently selected from hydrogen, —F, —Cl, —Br, —I, methyl, ethyl, propyl, butyl, pentyl, hexyl, cyclopropyl, cyclobutyl, cyclopentyl, cyclohexyl, 3- to 8-membered heterocyclyl, —NR^{10a}R^{10b}, —OR^{10a}, —CO₂R^{10a} or —C(O)NR^{10a}R^{10b}; each of methyl, ethyl, propyl, butyl, pentyl, hexyl, cyclopropyl, cyclobutyl, cyclopentyl, cyclohexyl or 3- to 8-membered heterocyclyl is optionally substituted with at least one R^{10c};
R^{10a} and R^{10b} are each independently selected from hydrogen, methyl, ethyl, propyl, butyl, pentyl, cyclopropyl, cyclobutyl, cyclopentyl, cyclohexyl, 3-membered heterocyclyl, 4-membered heterocyclyl, 5-membered heterocyclyl or 6-membered heterocyclyl;
R^{10c} is each independently selected from hydrogen, —F, —Cl, —Br, —I, methyl, ethyl, propyl, butyl, cyclopropyl, cyclobutyl, cyclopentyl, oxo (═O), —NR^{10e}R^{10f}, —OR^{10e} or —CN;
R^{10e} and R^{10f} are each independently selected from hydrogen, methyl, ethyl, propyl, cyclopropyl, cyclobutyl, cyclopentyl;
even more preferably, R¹⁰ is each independently selected from H, F, Cl, Br, methyl, ethyl, propyl (n-propyl or iso-propyl), butyl(n-butyl, sec-butyl, iso-butyl or tert-butyl), cyclopropyl, cyclobutyl, cyclopentyl, cyclohexyl,

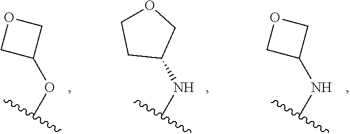 [SMILES: *OC1COC1 ; *N[C@@H]1CCOC1 ; *NC1COC1]

—COOH, —CONH₂, —CH₂OCH₃ or —CH₂OH.

Aspect 16. The compound of any one of the preceding Aspects, wherein the

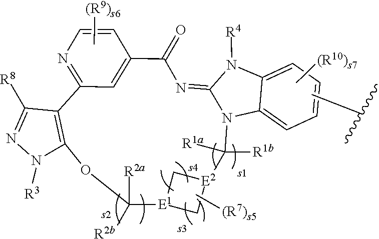

moiety is selected from

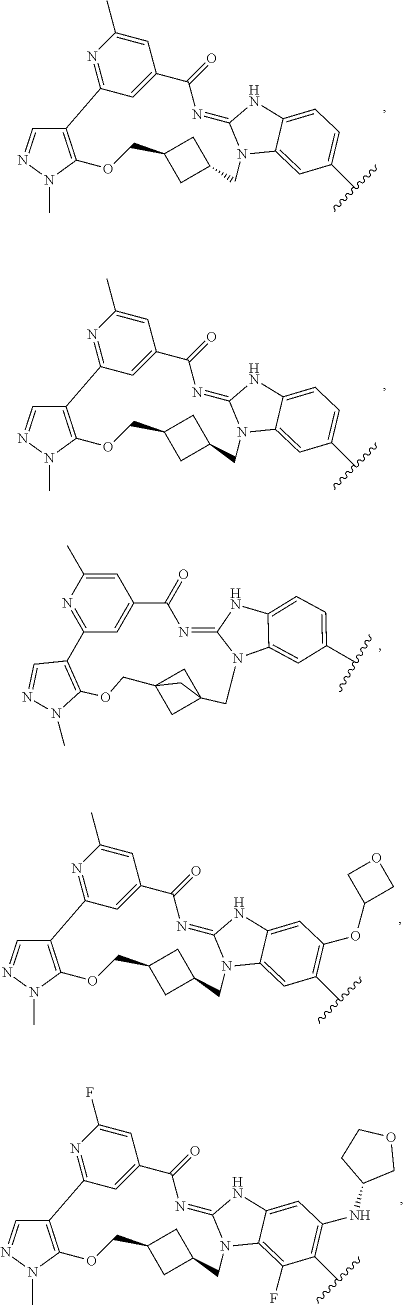 [SMILES: *c1ccc2c(c1)N1C[C@H]3C[C@H](COc4c(cnn4C)-c4cc(cc(C)n4)C(=O)/N=C/1N2)C3 ; *c1ccc2c(c1)N1C[C@H]3C[C@@H](COc4c(cnn4C)-c4cc(cc(C)n4)C(=O)/N=C/1N2)C3 ; *c1ccc2c(c1)N1CC34CC(COc5c(cnn5C)-c5cc(cc(C)n5)C(=O)/N=C/1N2)(C3)C4 ; *c1cc2c(cc1OC1COC1)N/C1=N\C(=O)c3cc(C)nc(c3)-c3cnn(C)c3OC[C@H]3C[C@@H](CN12)C3 ; *c1c(N[C@@H]2CCOC2)cc2c(c1F)N1C[C@H]3C[C@@H](COc4c(cnn4C)-c4cc(cc(F)n4)C(=O)/N=C/1N2)C3]

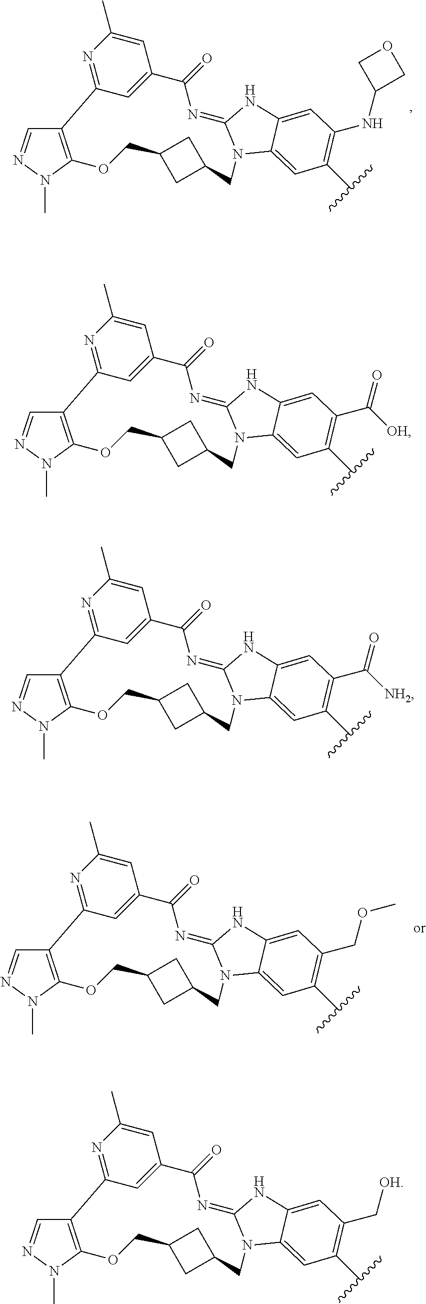 [SMILES: *c1cc2c(cc1NC1COC1)N/C1=N\C(=O)c3cc(C)nc(c3)-c3cnn(C)c3OC[C@H]3C[C@@H](CN12)C3 ; *c1cc2c(cc1C(=O)O)N/C1=N\C(=O)c3cc(C)nc(c3)-c3cnn(C)c3OC[C@H]3C[C@@H](CN12)C3 ; *c1cc2c(cc1C(N)=O)N/C1=N\C(=O)c3cc(C)nc(c3)-c3cnn(C)c3OC[C@H]3C[C@@H](CN12)C3 ; *c1cc2c(cc1COC)N/C1=N\C(=O)c3cc(C)nc(c3)-c3cnn(C)c3OC[C@H]3C[C@@H](CN12)C3 ; *c1cc2c(cc1CO)N/C1=N\C(=O)c3cc(C)nc(c3)-c3cnn(C)c3OC[C@H]3C[C@@H](CN12)C3]

Aspect 17. The compound of any one of the preceding Aspects, wherein the

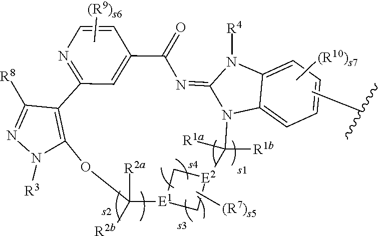

moiety is selected from

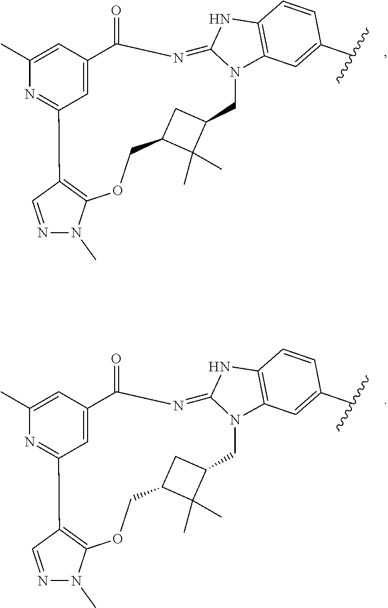 [SMILES: *c1ccc2c(c1)N1C[C@@H]3C[C@H](COc4c(cnn4C)-c4cc(cc(C)n4)C(=O)/N=C/1N2)C3(C)C ; "," ; *c1ccc2c(c1)N1C[C@H]3C[C@@H](COc4c(cnn4C)-c4cc(cc(C)n4)C(=O)/N=C/1N2)C3(C)C ; "."]

Aspect 18. The compound of any one of the preceding Aspects, wherein R^{11a}, R^{11b}, R^{11c}, R^{11d}, R^{12a}, R^{12b}, R^{12c} and R^{12d} are each independently oxo, hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl; each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl is optionally substituted with at least one substituent selected from hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy or —CN;
preferably, R^{11a}, R^{11b}, R^{11c}, R^{11d}, R^{12a}, R^{12b}, R^{12c} and R^{12d} are each independently oxo, hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl; preferably, R^{11a}, R^{11b}, R^{11c}, R^{11d}, R^{12a}, R^{12b}, R^{12c} and R^{12d} are each independently oxo, hydrogen, F, Cl, Br, I, methyl, ethyl or propyl; more preferably, R^{11a}, R^{11b}, R^{11c}, R^{11d}, R^{12a}, R^{12b}, R^{12c} and R^{12d} are each independently hydrogen or methyl.

Aspect 19. The compound of any one of the preceding Aspects, wherein L¹ is selected from —O—, —C(O)—, —N(R′)—, *^{L1}-C(O)N(R′)—**^{L1}, *^{L1}-C(O)O—**^{L1}, *^{L1}-N(Ra)C(O)—**^{L1}, *^{L1}-OC(O)—**^{L1},

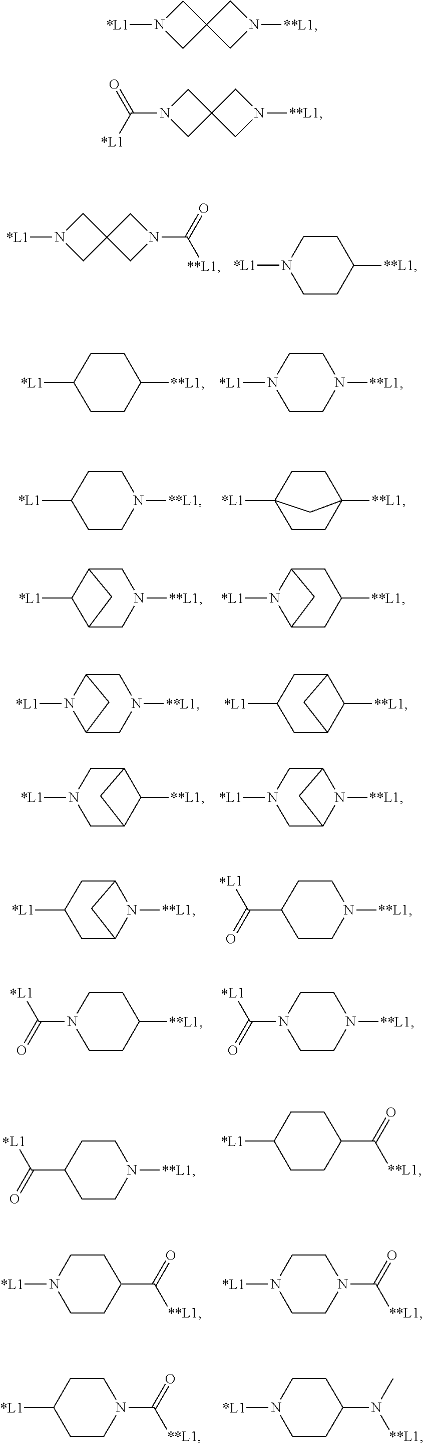

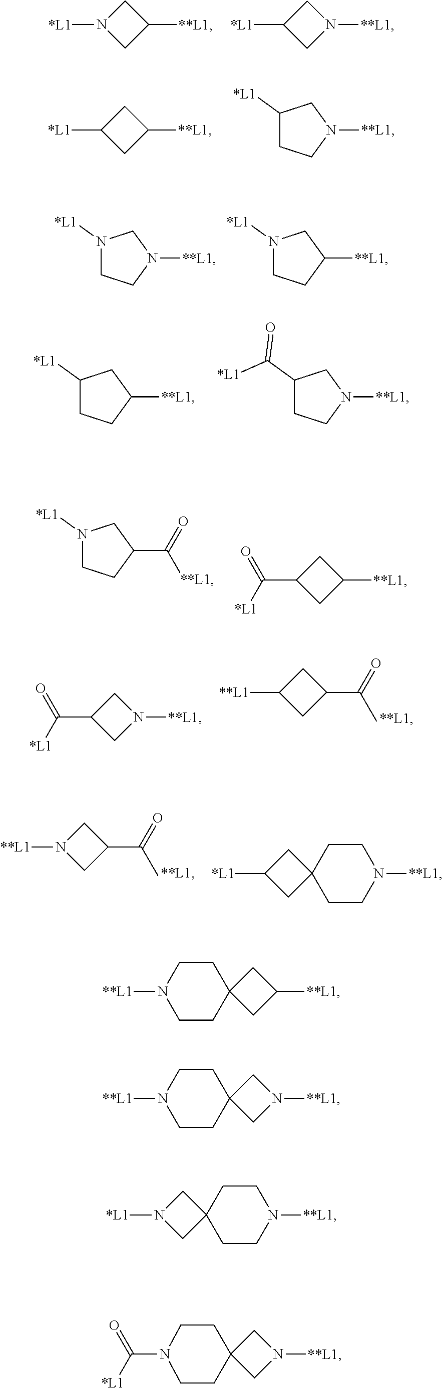

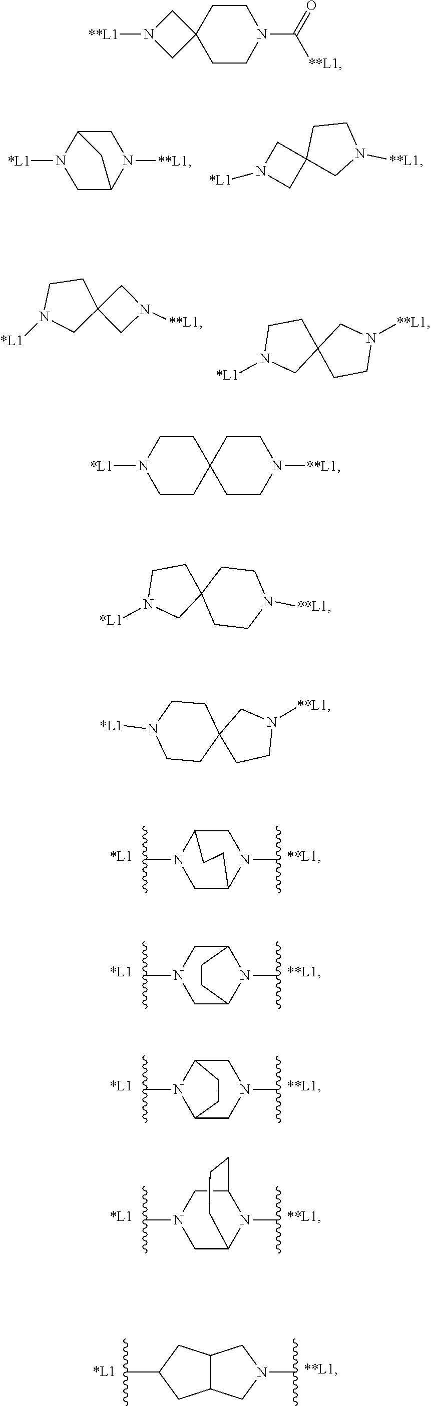

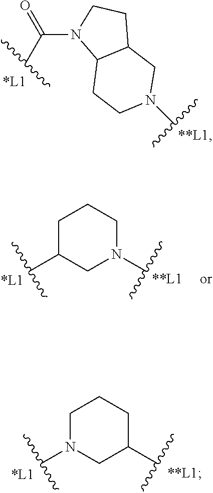 [SMILES: *C(=O)N1CCC2CN(*)CCC21 ; *C1CCCN(*)C1 ; *C1CCCN(*)C1]

wherein each of said

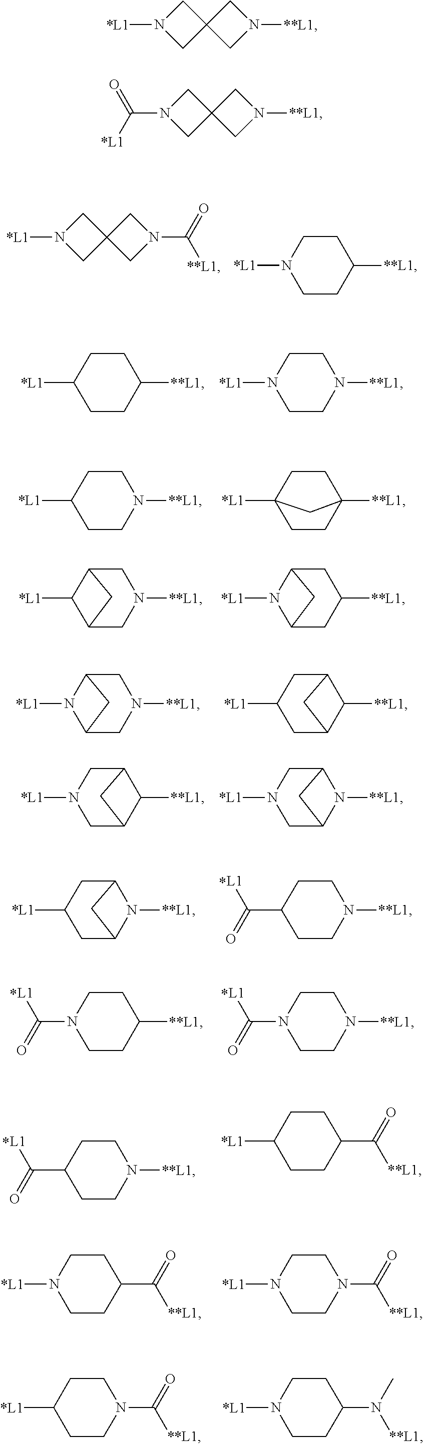

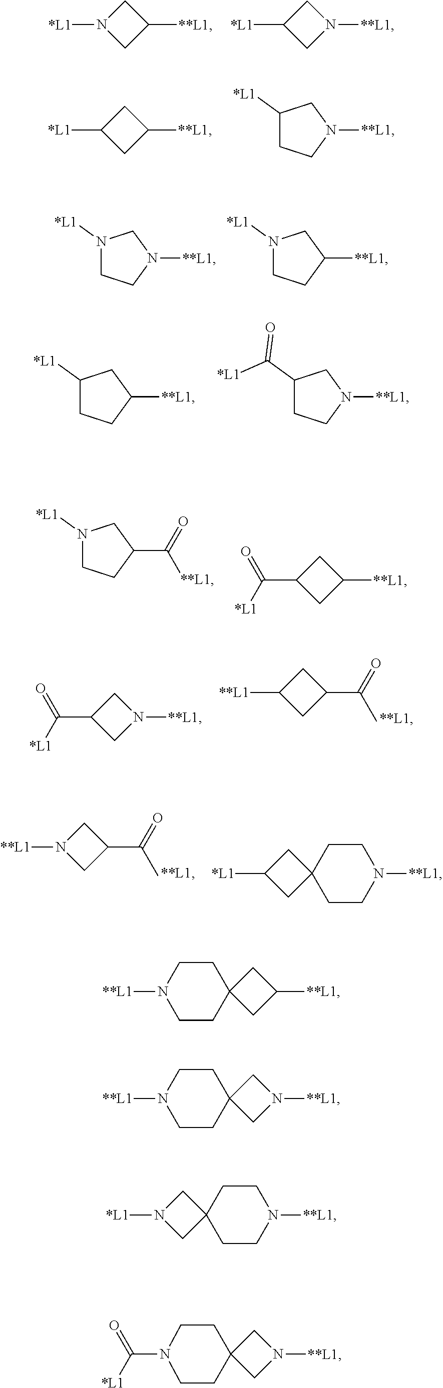

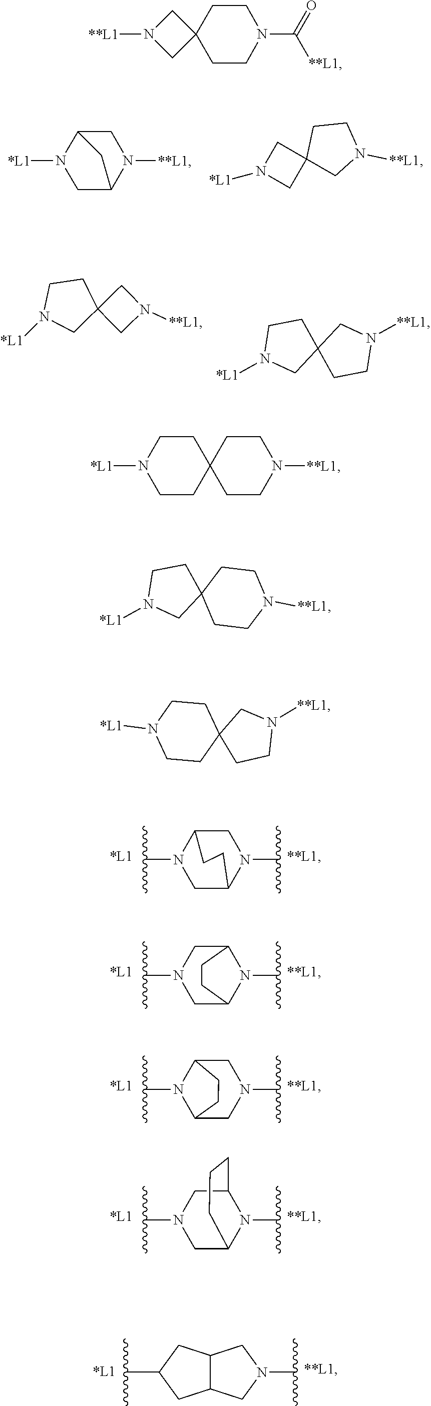

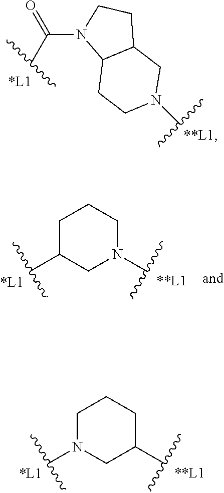 [SMILES: *C(=O)N1CCC2CN(*)CCC21 ; *C1CCCN(*)C1 ; *C1CCCN(*)C1]

is optionally substituted with at least one R^{L1c};
each of said R^{L}C is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{LCa},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 20. The compound of any one of the preceding Aspects, wherein L¹ is selected from —O—, —N(CH₃)—, —C(O)—, —NH—, *^{L1}-C(O)N(CH₃)—**^{L1}, *^{L1}-C(O)NH—**^{L1}, *^{L1}-C(O)O—**^{L1}, *^{L1}-C(O)N(C₂H₅)—**^{L1}, *^{L1}-C(O)N(C₃H₇)**^{L1}, *^{L1}-N(CH₃)C(O)—**^{L1}, *^{L1}-NHC(O)—**^{L1}, *^{L1}—OC(O)—**L, *^{L1}-N(C₂H₅)C(O)**^{L1}, *^{L1}-N(C₃H₇)C(O)—**^{L1},

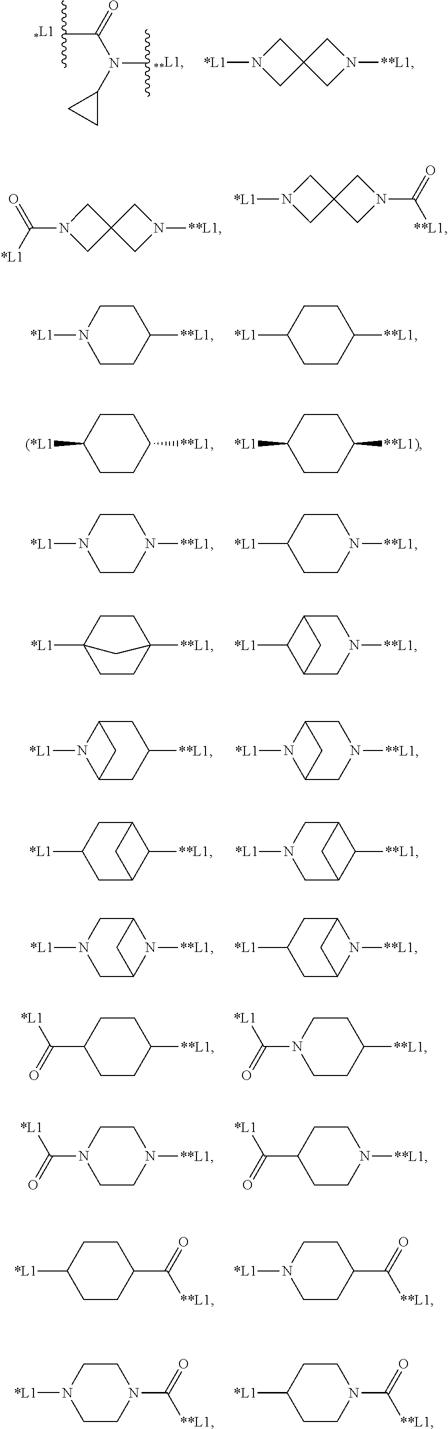

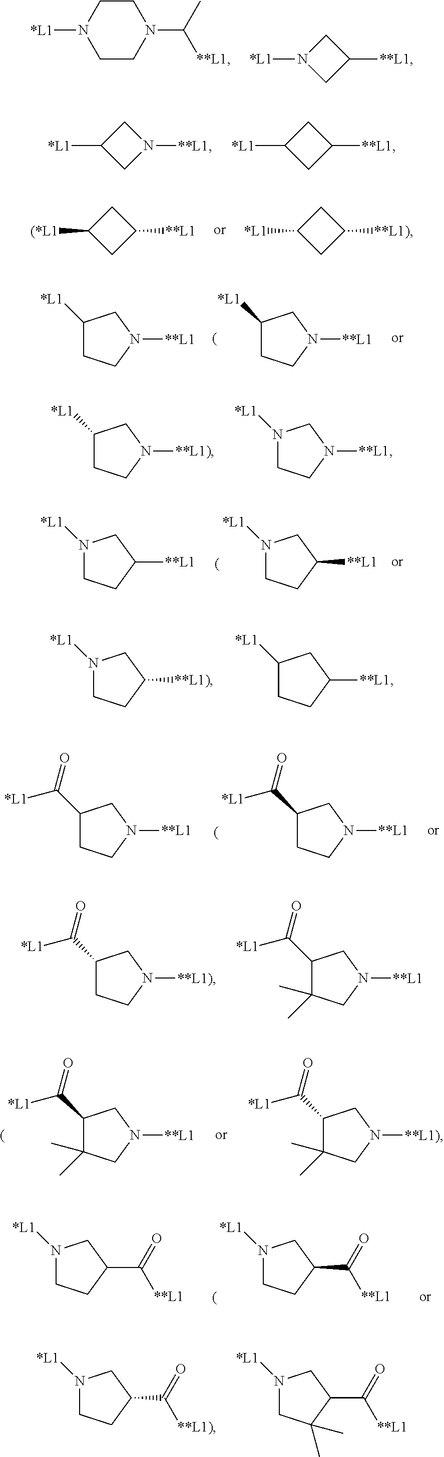

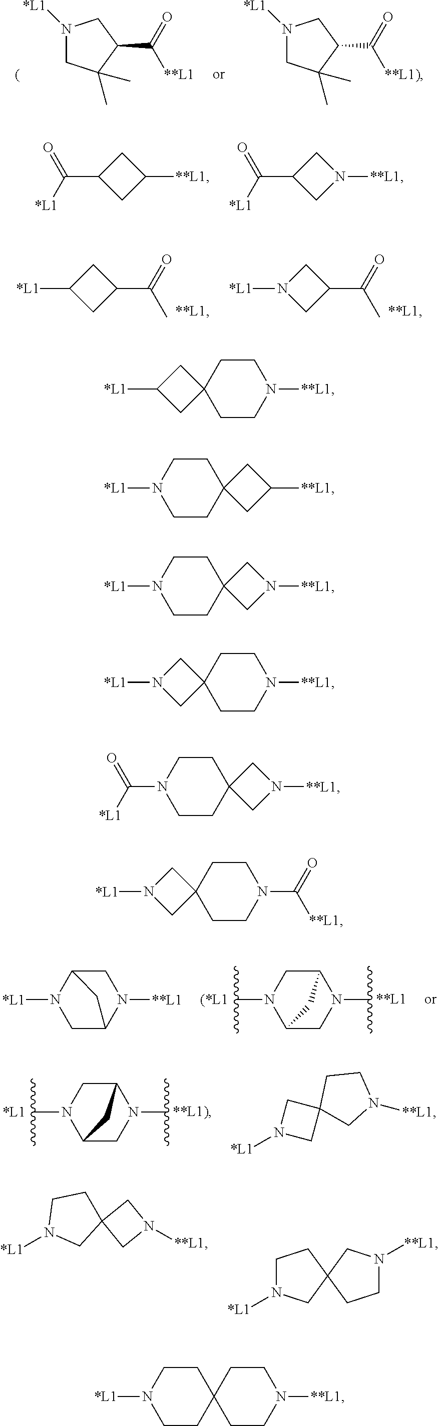

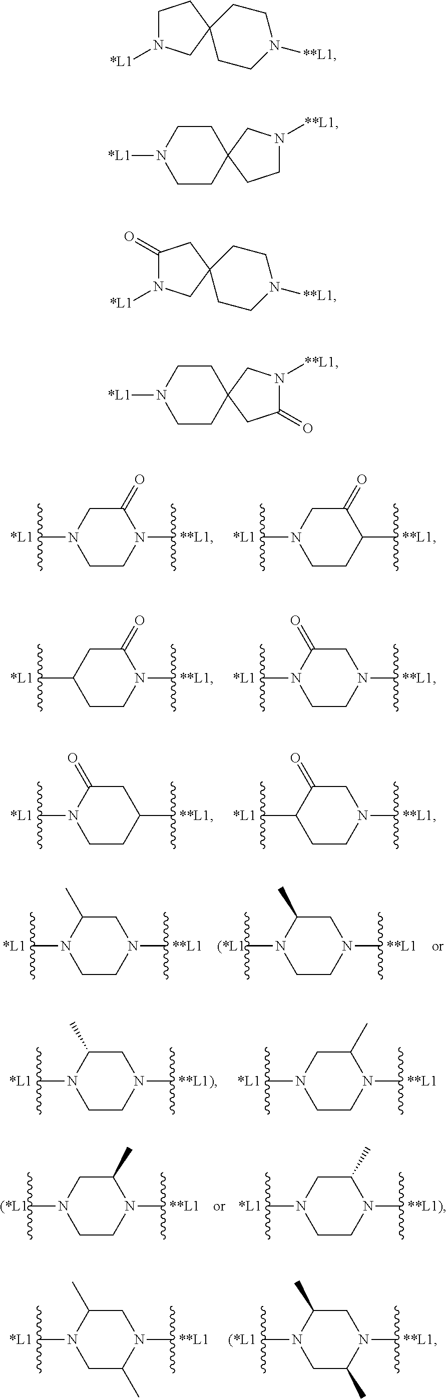

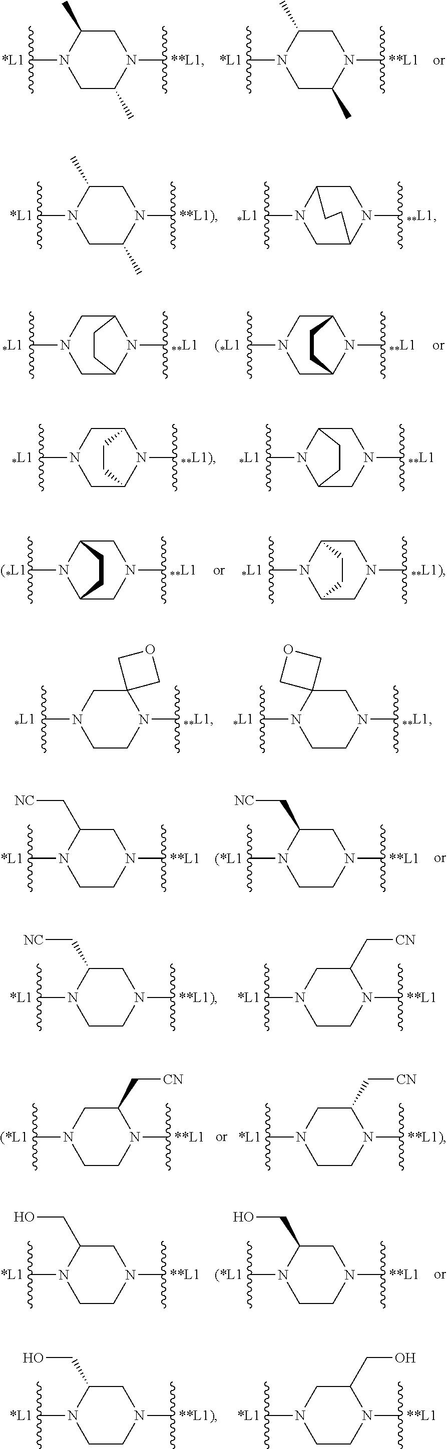

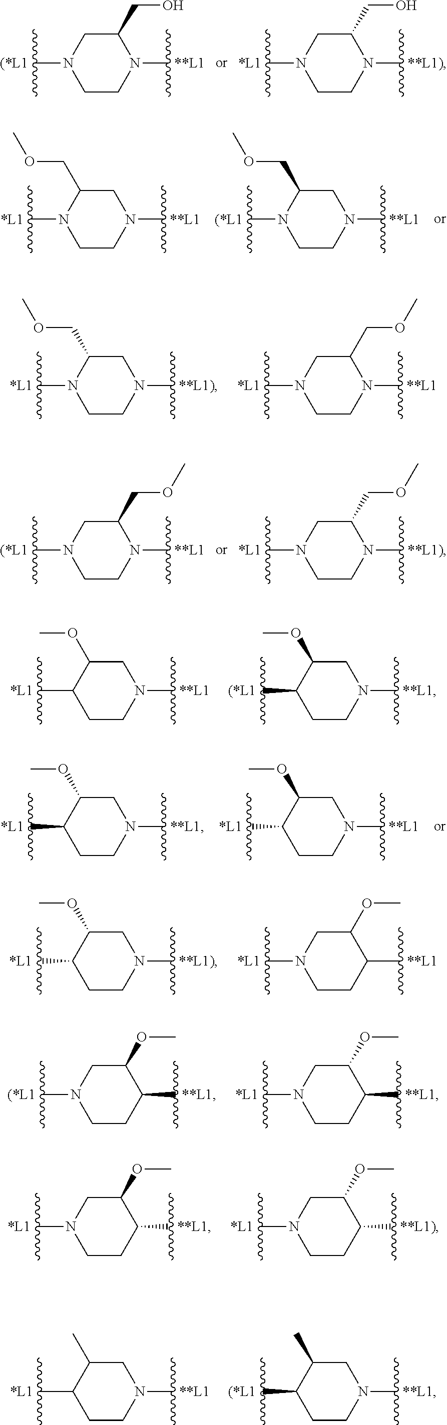

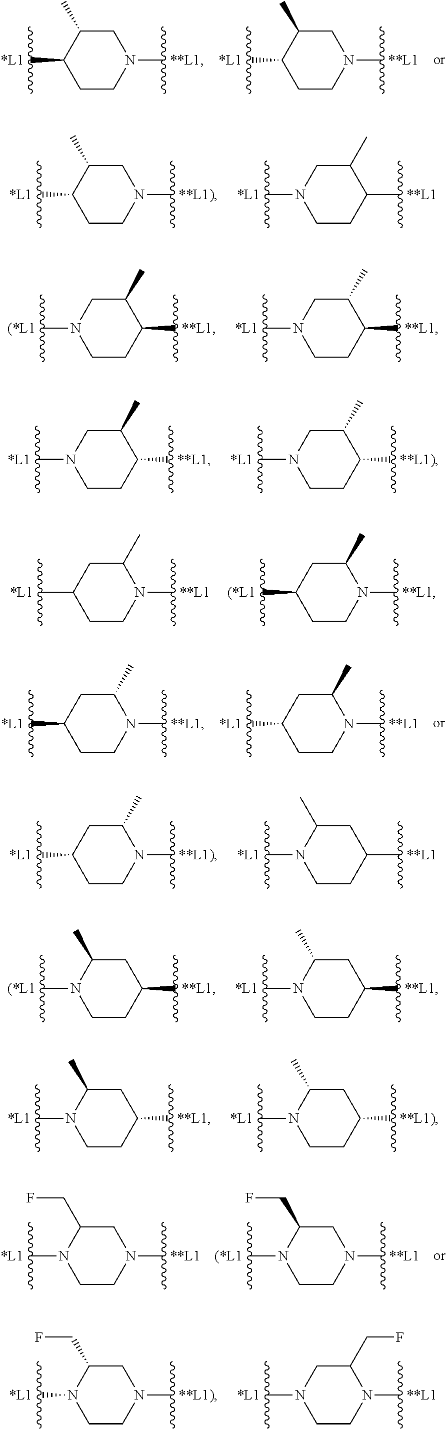

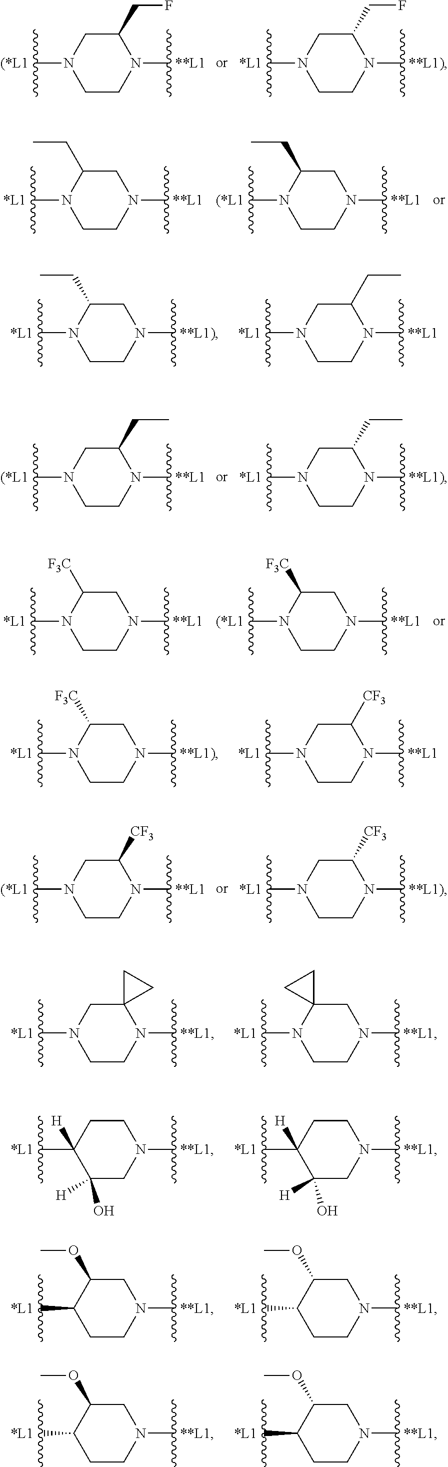

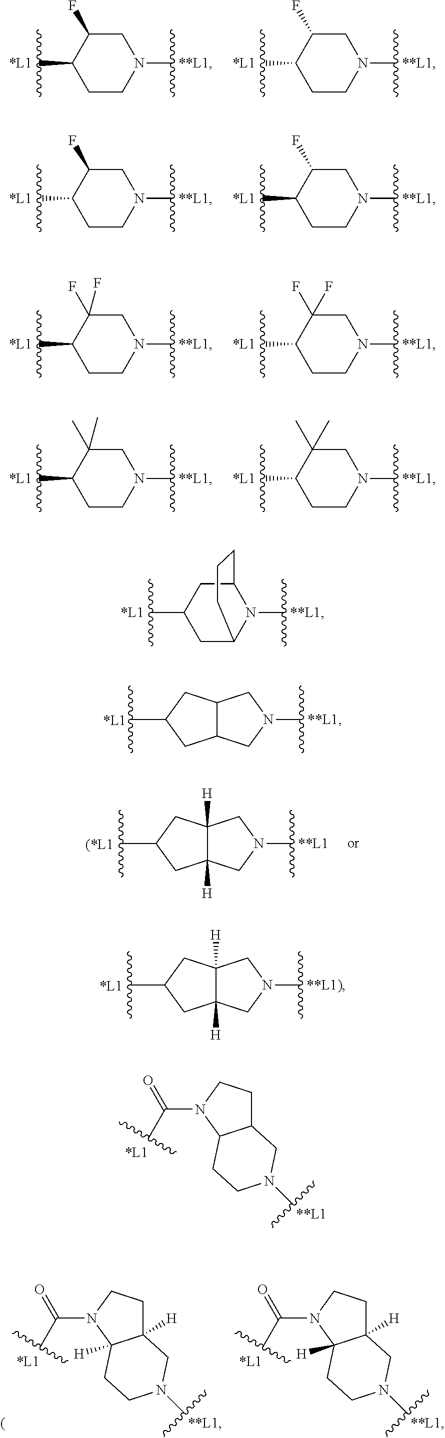

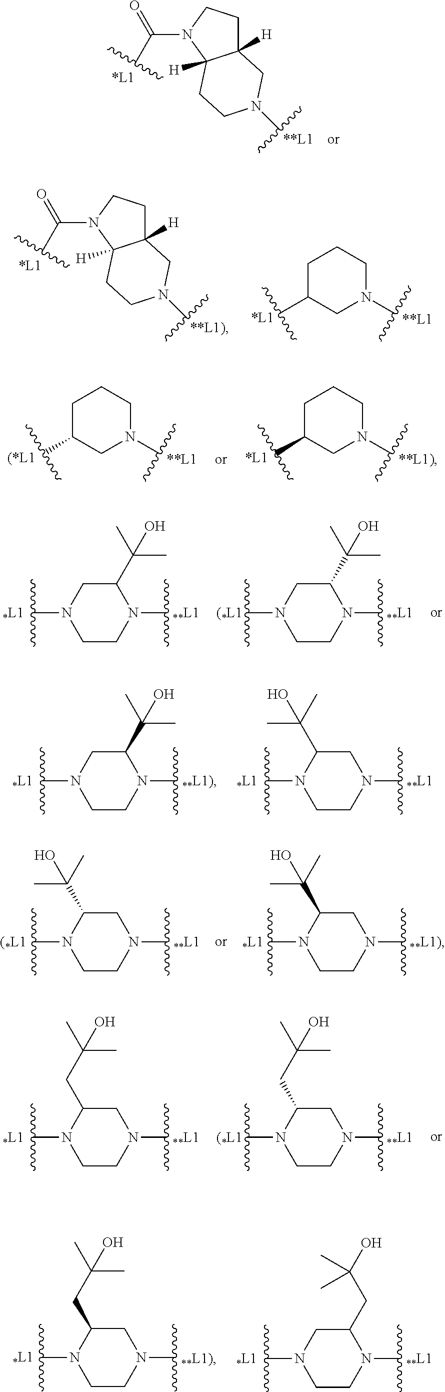

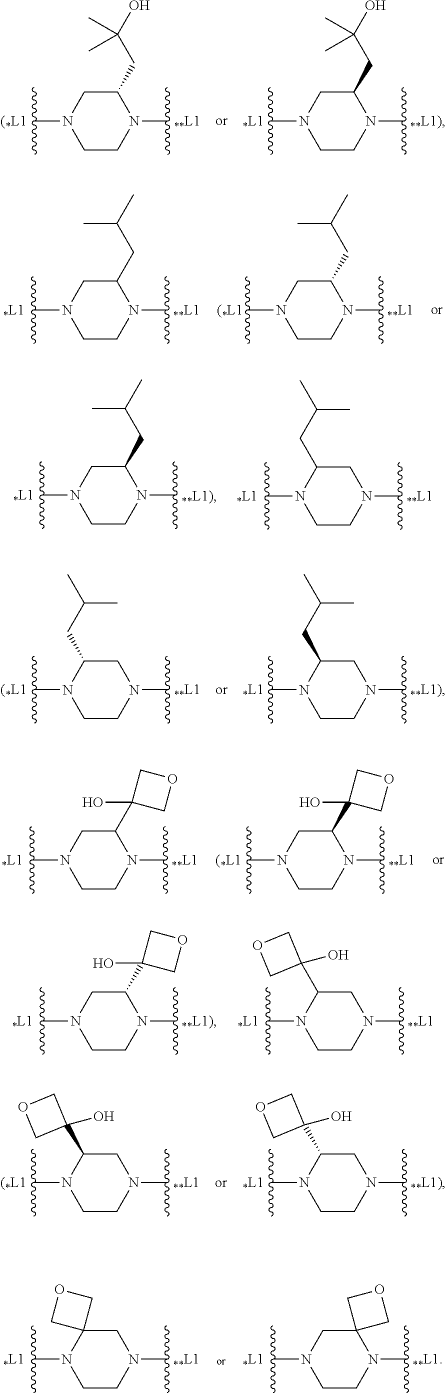

Aspect 21. The compound of any one of the preceding Aspects, wherein L² is selected from —O—, —C(O)—, —N(R^{a})—, -*^{L2}-C(O)N(R^{a})—**^{L2}, *^{L2}-C(O)O—**^{L2}, *^{L2}-N(R^{a})C(O)—*^{L2}, *^{L2}-OC(O)—**^{L2},

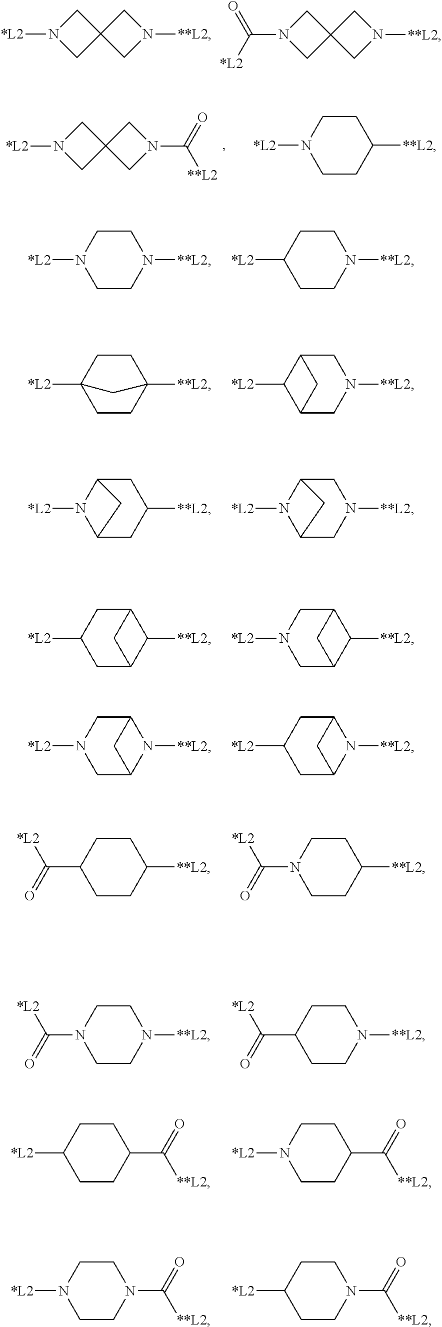

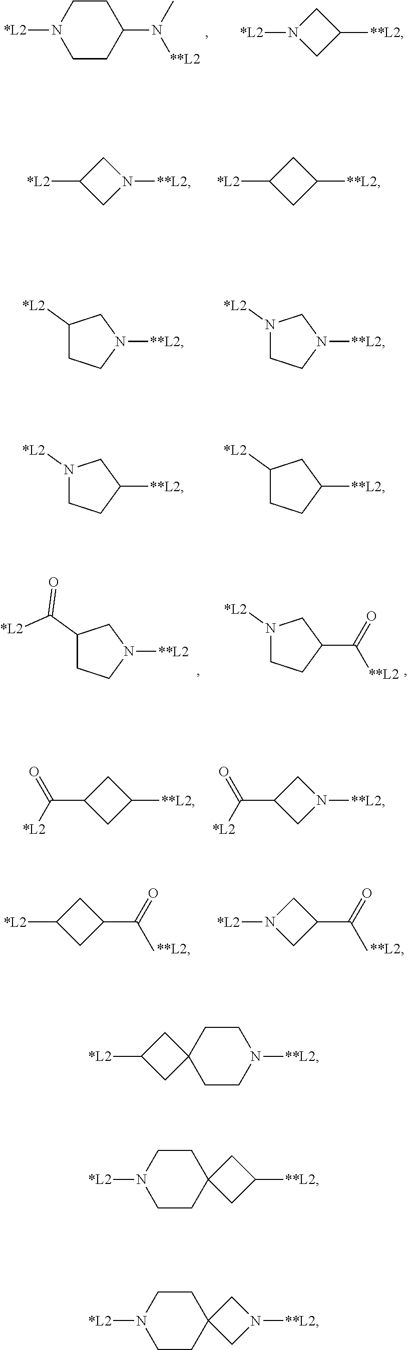

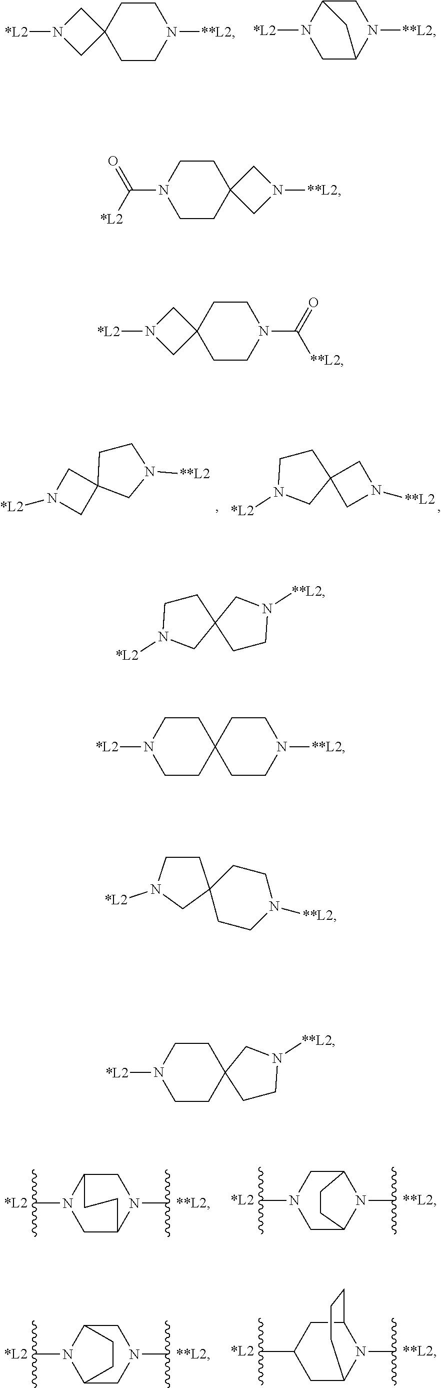

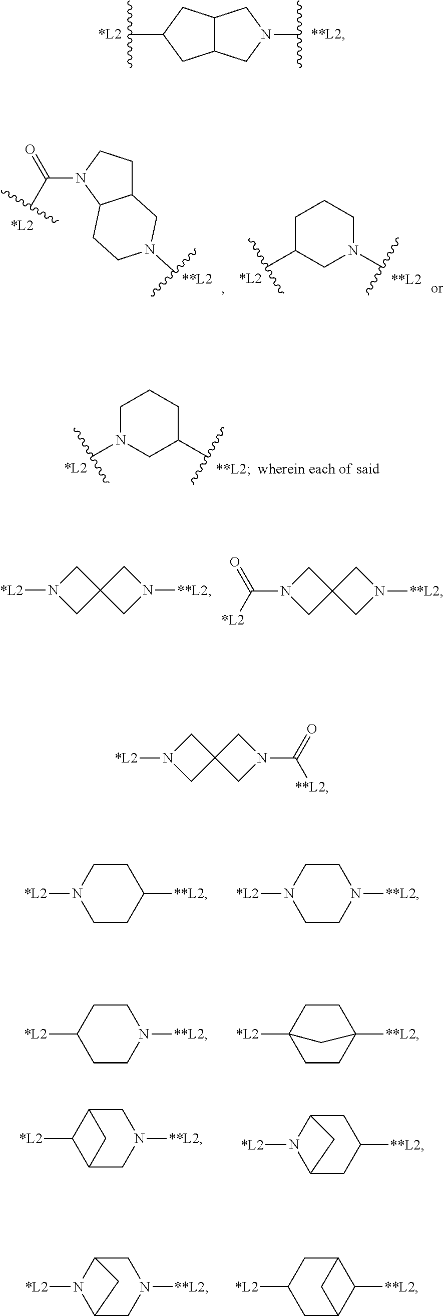

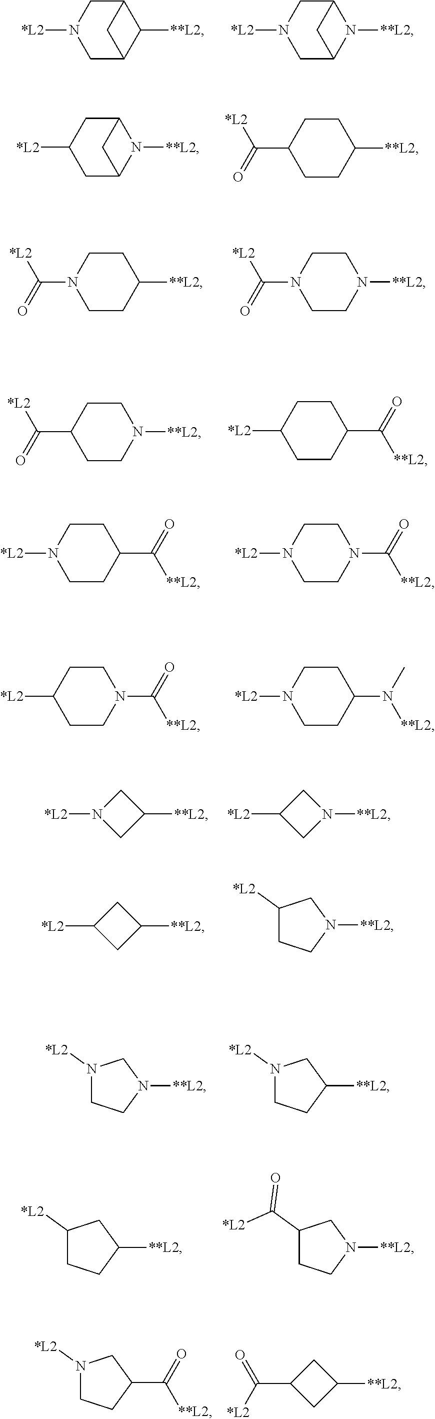

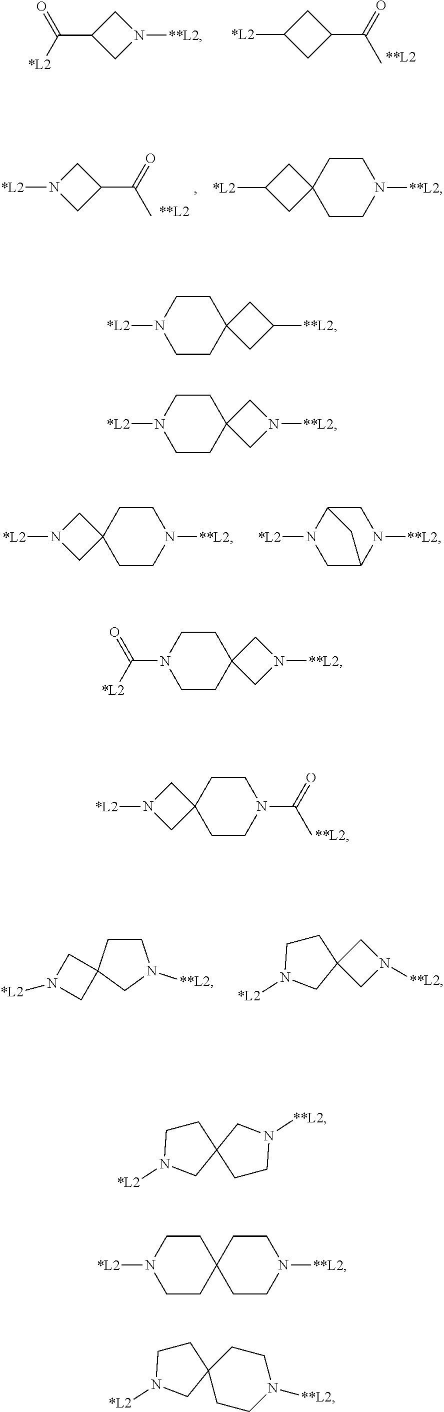

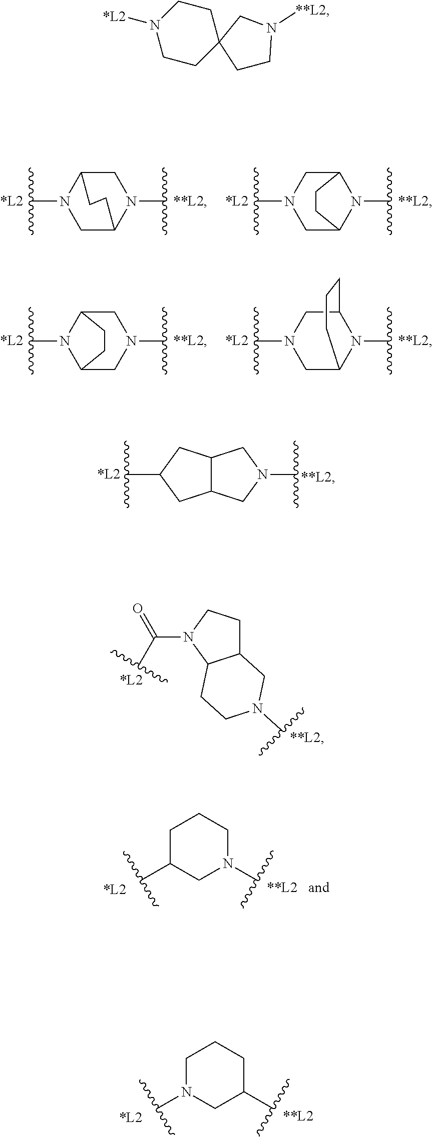

is optionally substituted with at least one R^{L2e};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2}, together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 22. The compound of any one of the preceding Aspects, wherein L² is selected from —O—, —N(CH₃)—, —C(O)—, —NH—, *^{L2}-C(O)N(CH₃)**^{L2}, *^{L2}-C(O)NH—**^{L2}, *^{L2}-C(O)O—**^{L2}, *^{L2}-C(O)N(C₂H₅)—**^{L2}, *^{L2}—C(O)N(C₃H₇)—**^{L2}, *^{L2}—N(CH₃)C(O)**^{L2}, *^{L2}-NHC(O)**^{L2}, *^{L2}-C(O)**^{L2}, *^{L2}-N(C₂H₅)C(O)**^{L2}, *^{L2}-N(C₃H₇)C(O)—**^{L2},

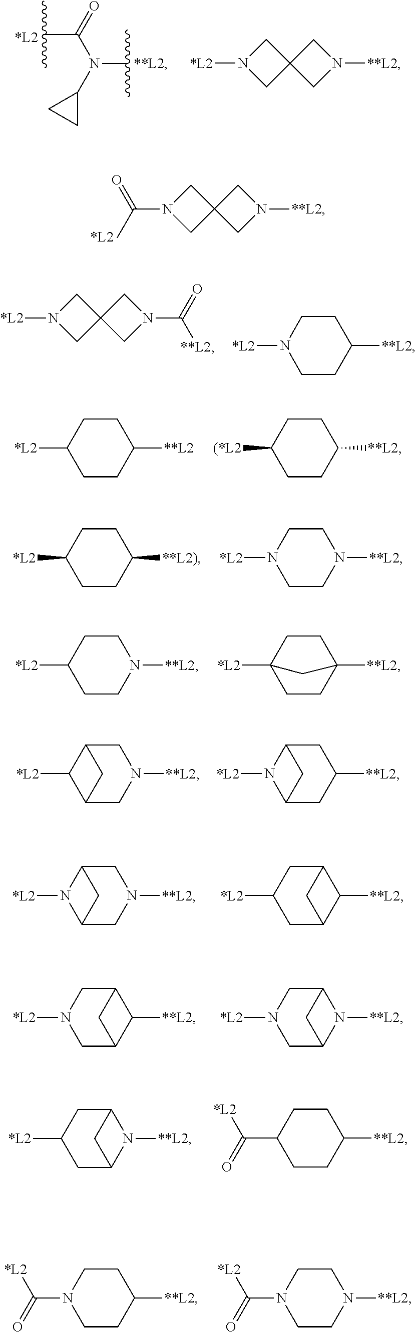

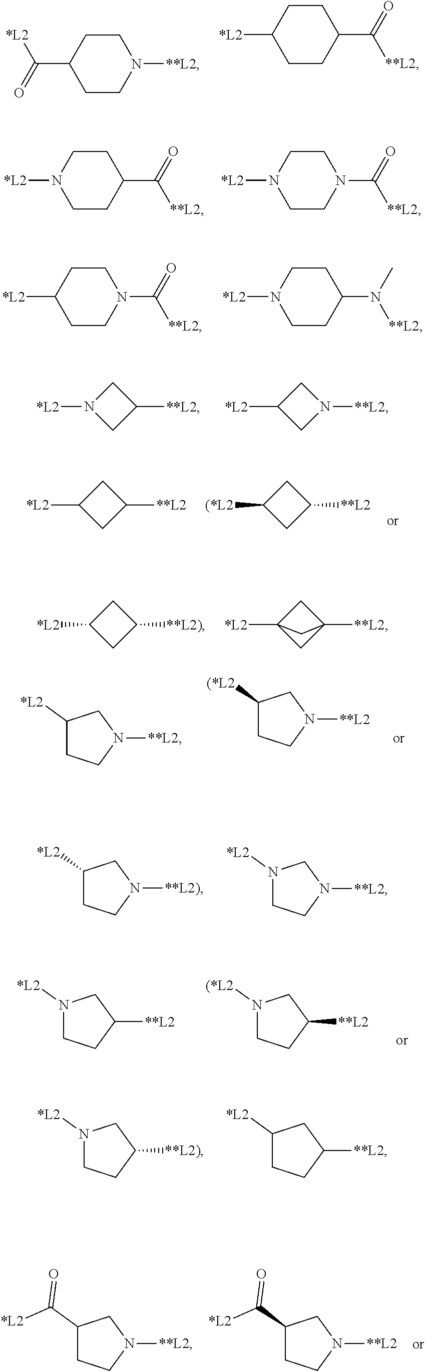

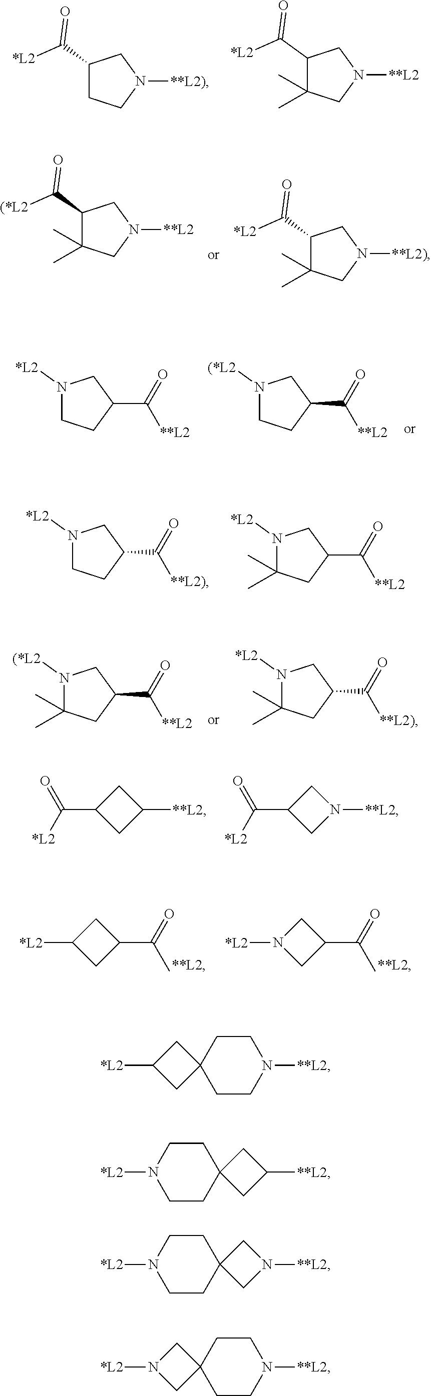

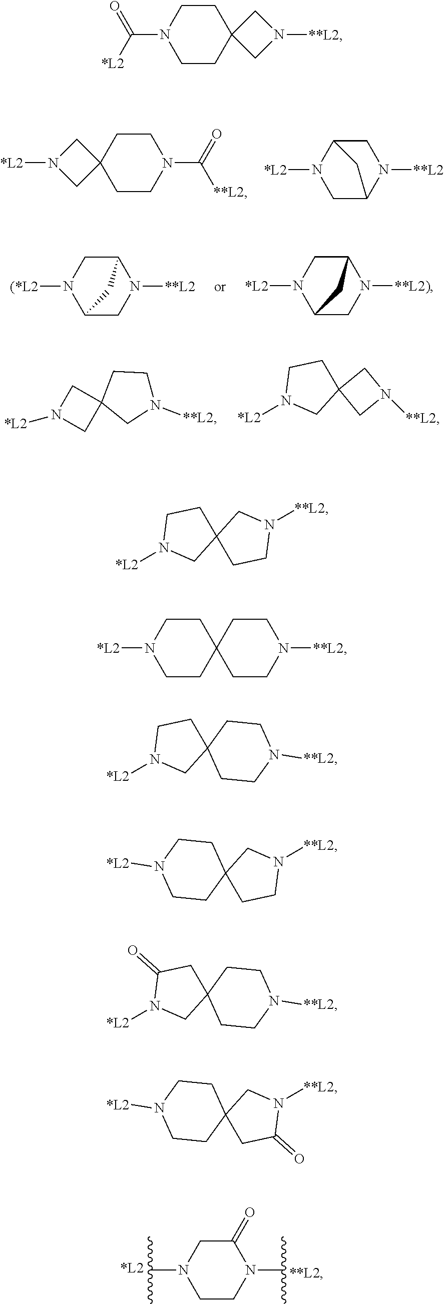

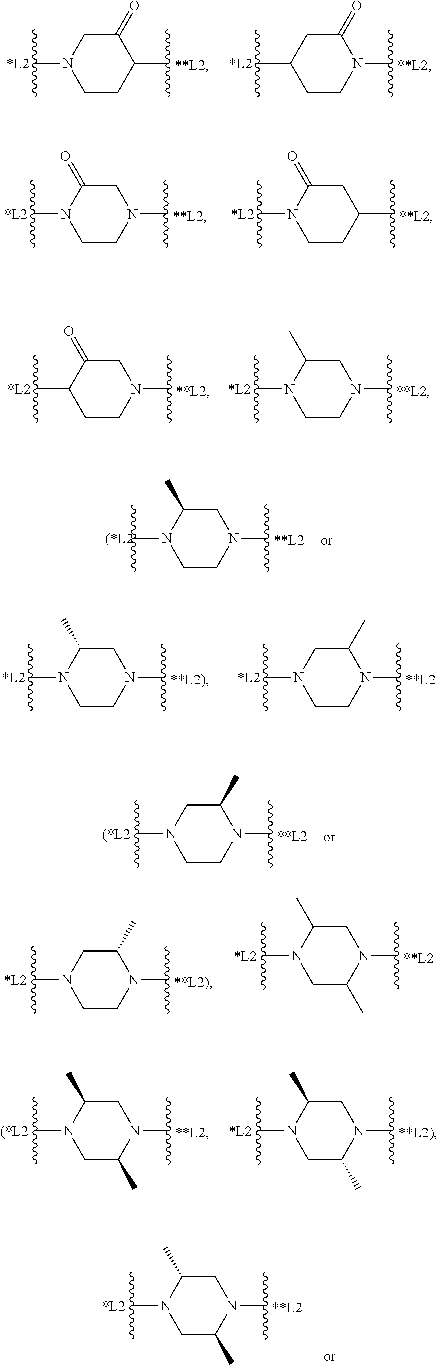

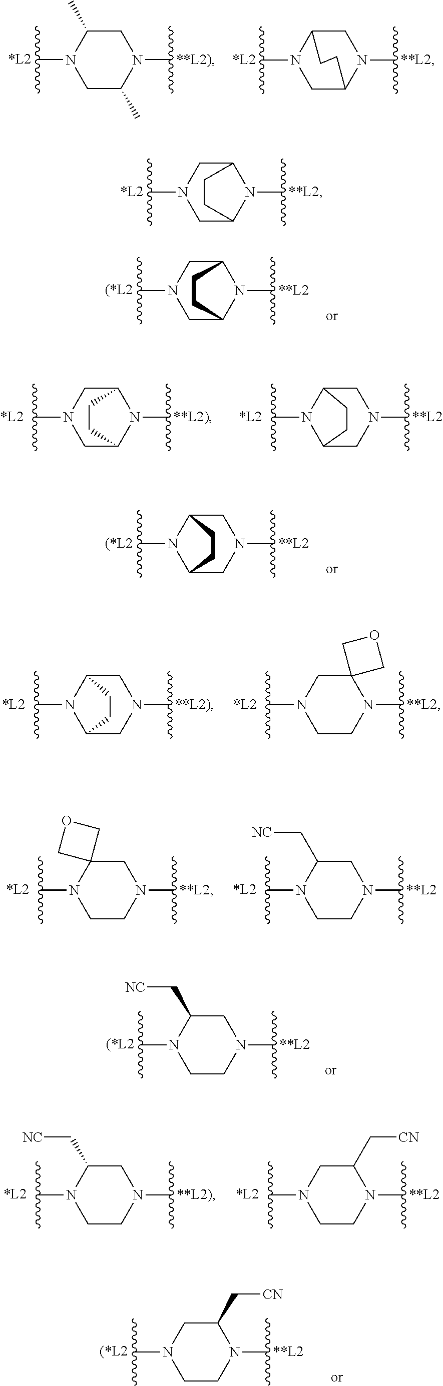

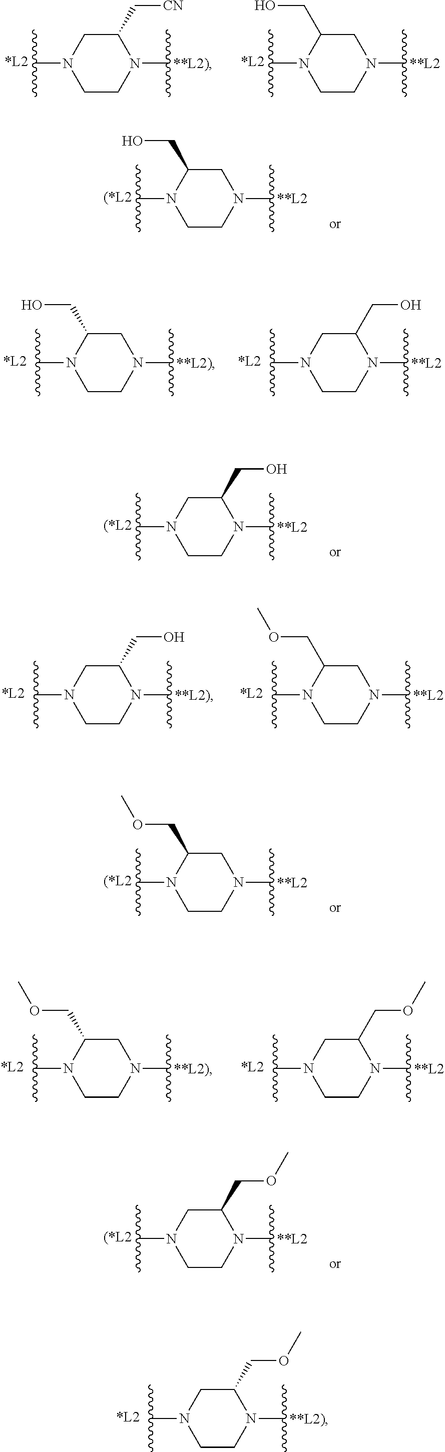

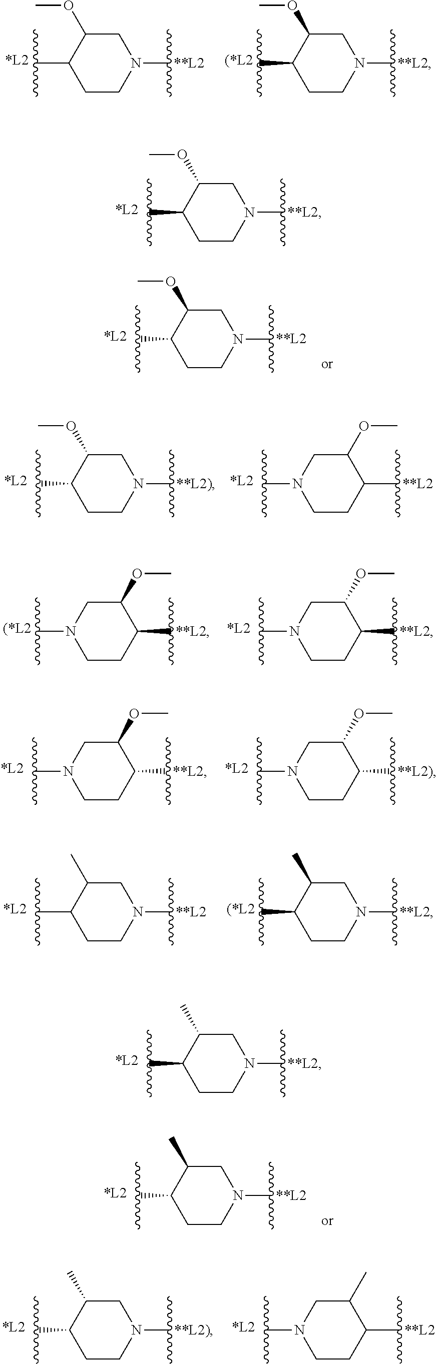

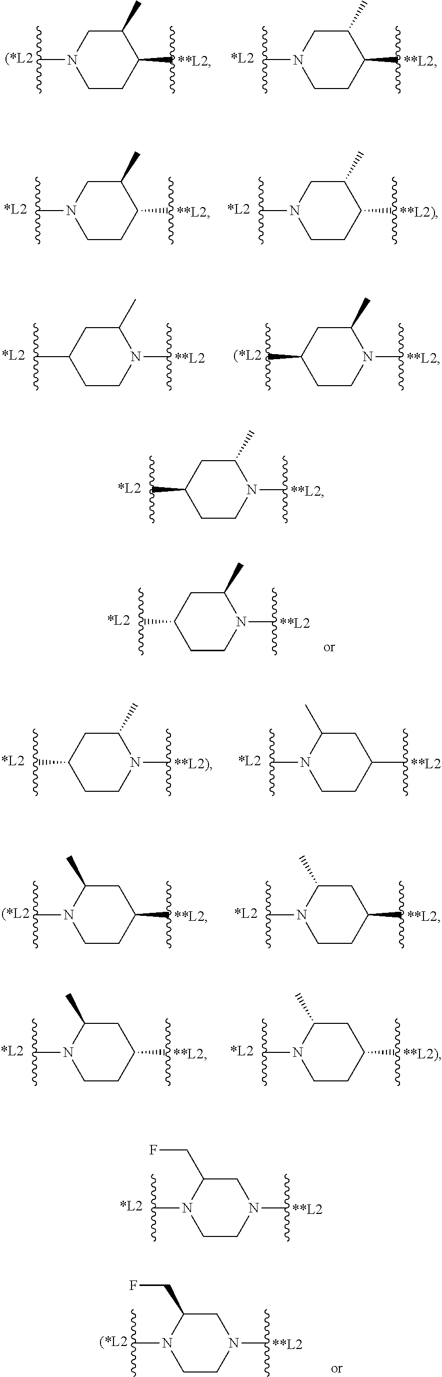

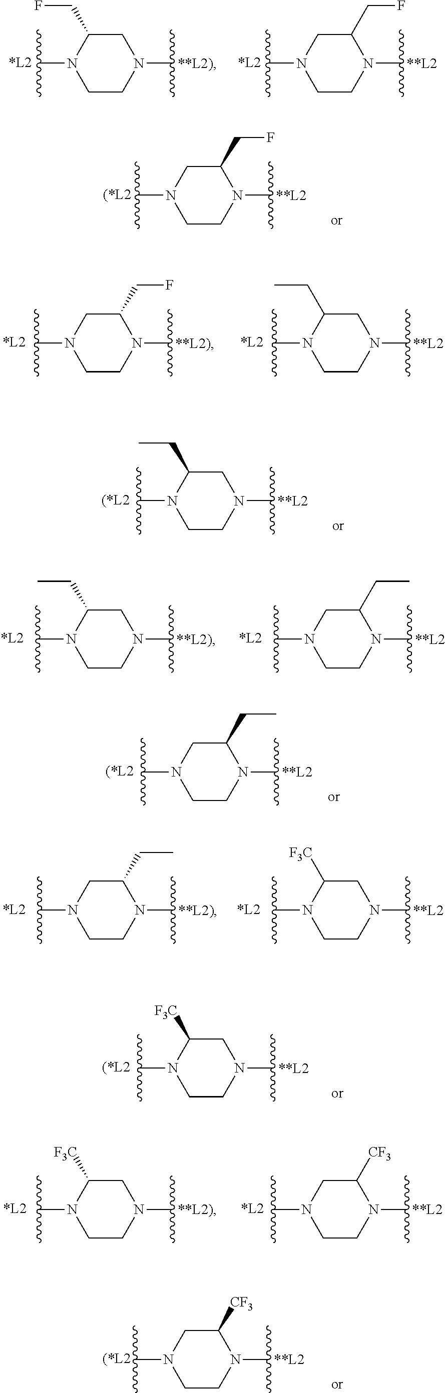

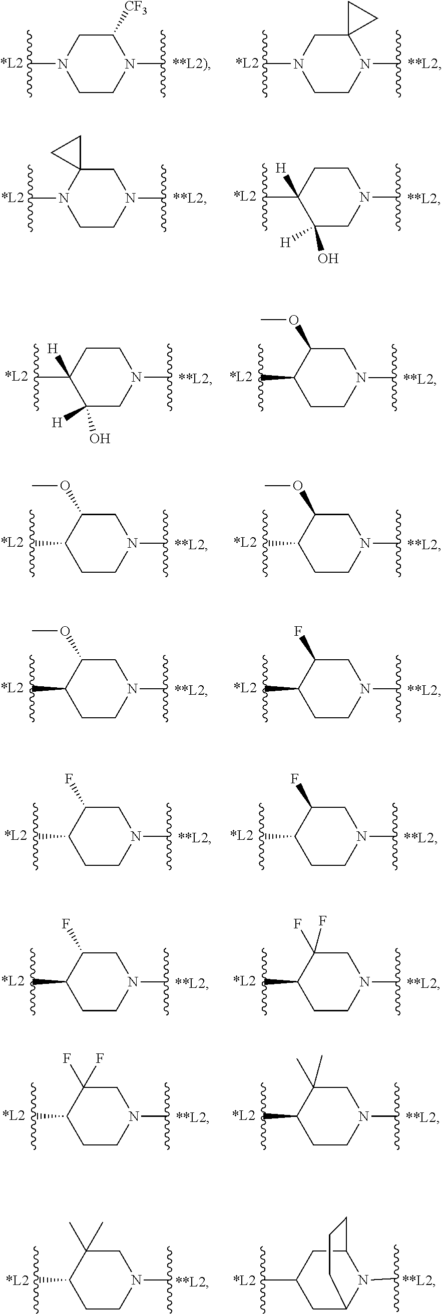

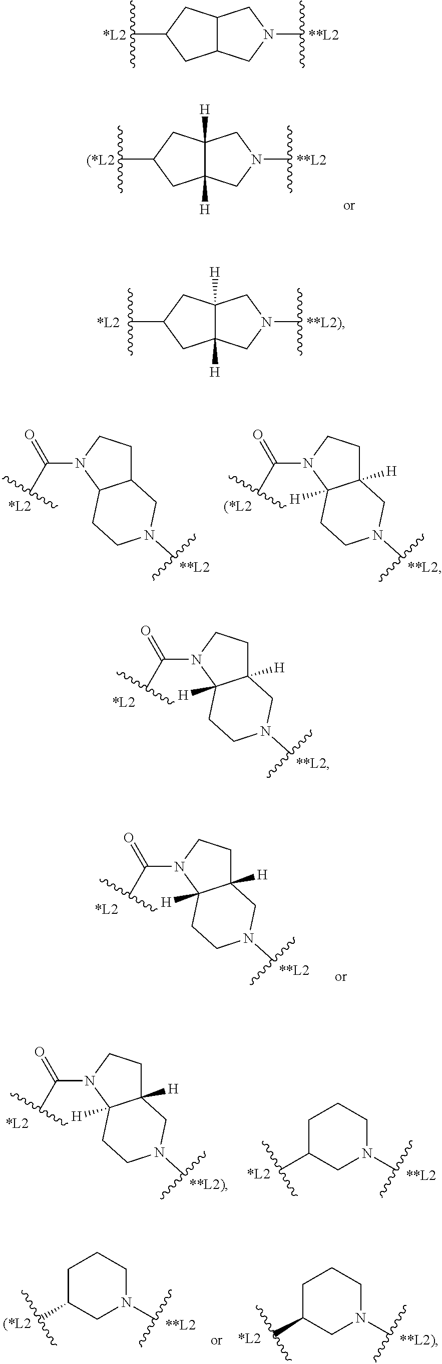

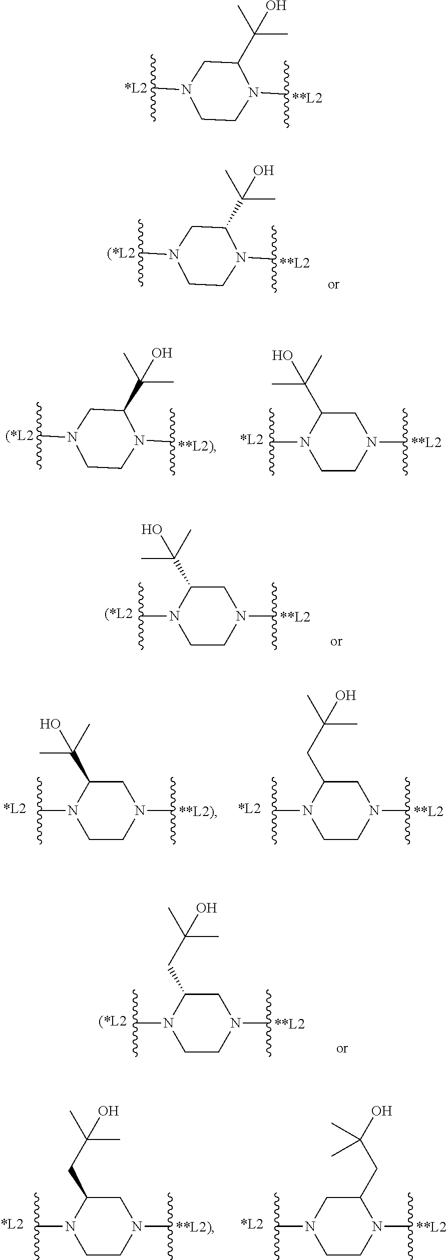

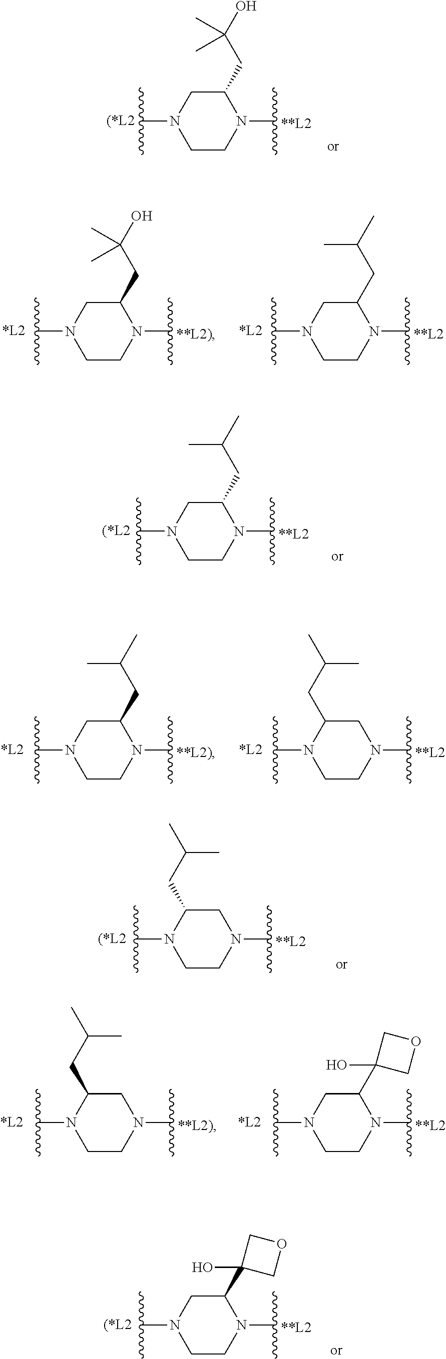

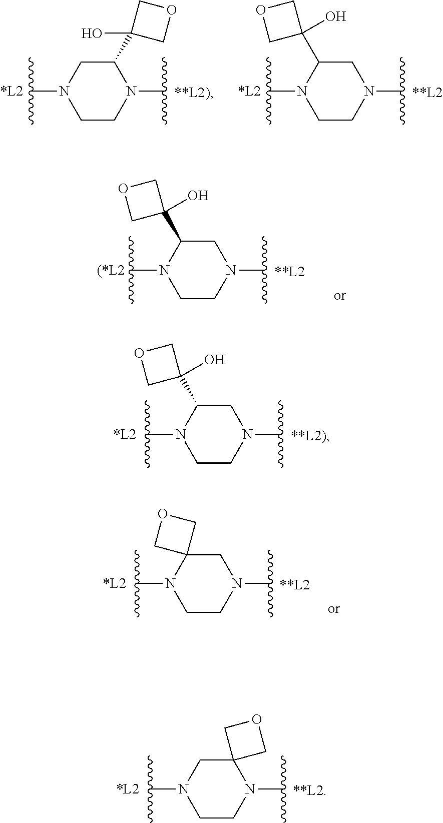

Aspect 23. The compound of any one of the preceding Aspects, wherein L³ is selected from —O—, —N(R^{a})—, —C(O)—, *^{L3}-C(O)N(R^{a})—**^{L3}, *^{L3}-C(O)O—**^{L3}, *^{L3}-N(R^{a})C(O)—**^{L3}, *^{L3}-OC(O)—**^{L3},

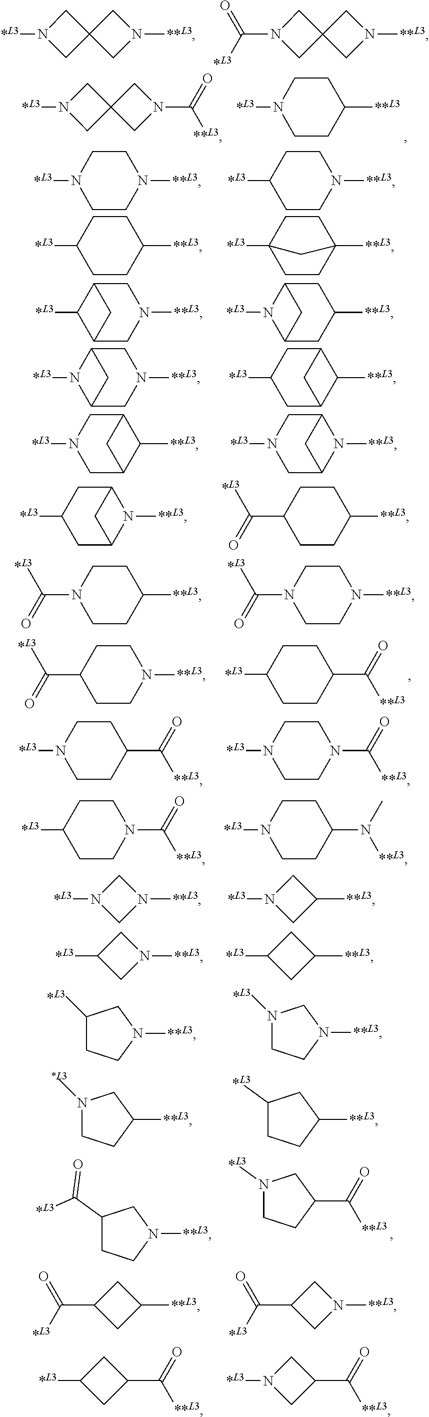

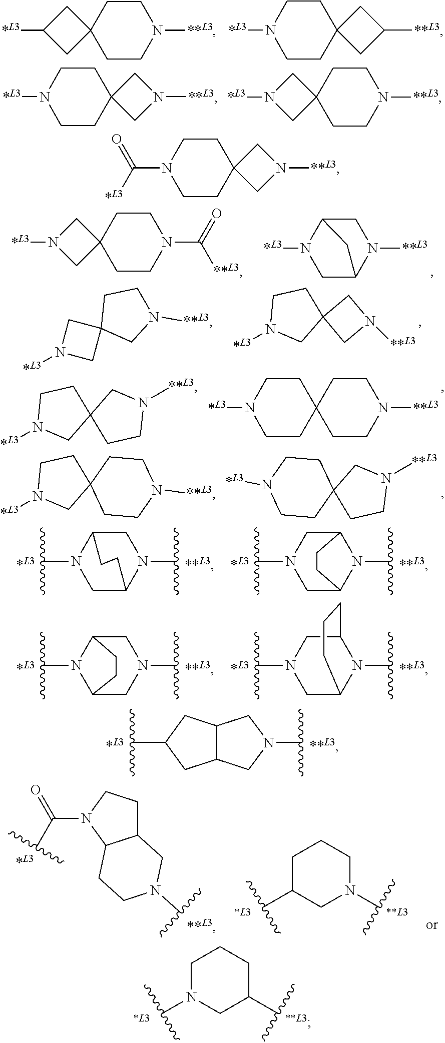

wherein each of said

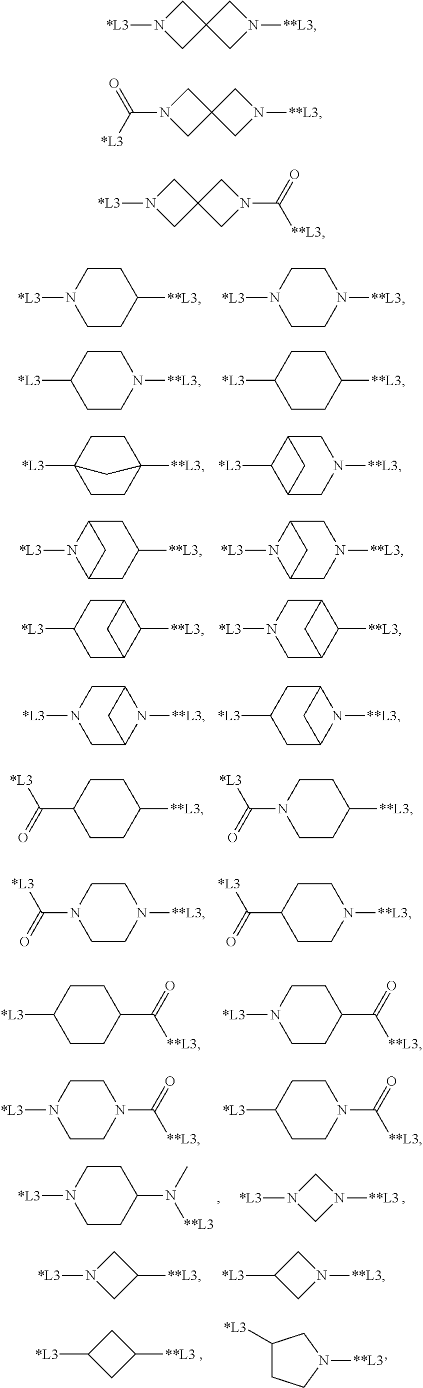

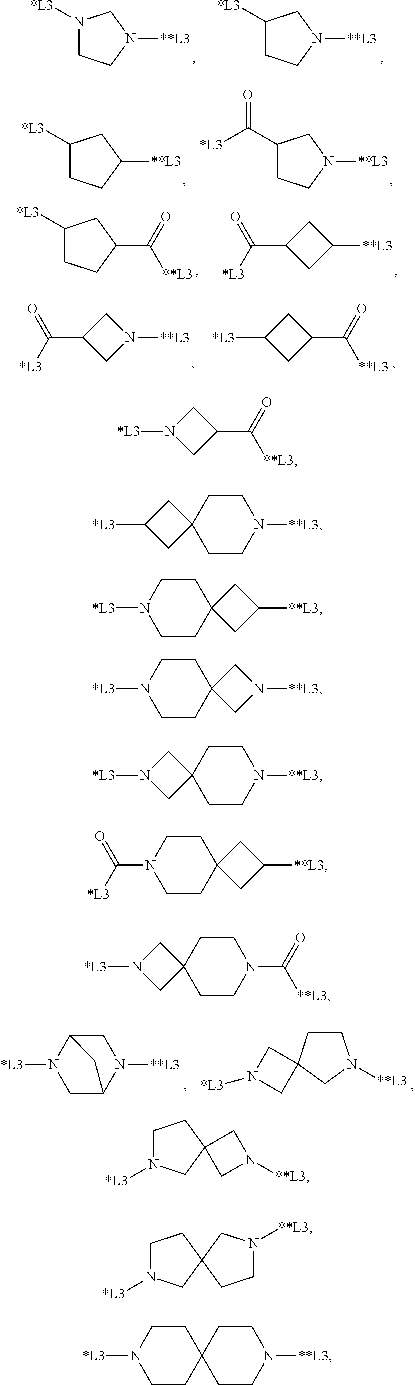

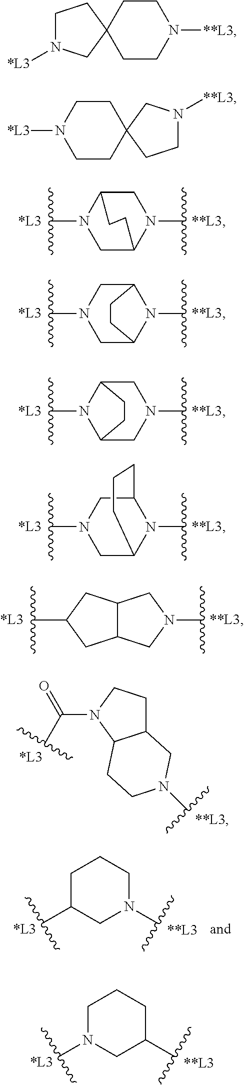

is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 24. The compound of any one of the preceding Aspects, wherein L³ is selected from —O—, —N(CH₃)—, —C(O)—, —NH—, *^{L3}-C(O)N(CH₃)—**^{L3}, *^{L3}-C(O)NH—**^{L3}, *^{L3}-C(O)O—**^{L3}, *^{L3}-C(O)N(C₂H₅)—**^{L3}, *^{L3}-C(O)N(C₃H₇)—**^{L3}, *^{L3}—N(CH₃)C(O)—**^{L3}, *^{L3}-NHC(O)—**^{L3}, *^{L3}-OC(O)—**^{L3}, *^{L3}-N(C₂H₅)C(O)—**^{L3}, *^{L3}-N(C₃H₇)C(O)—**^{L3},

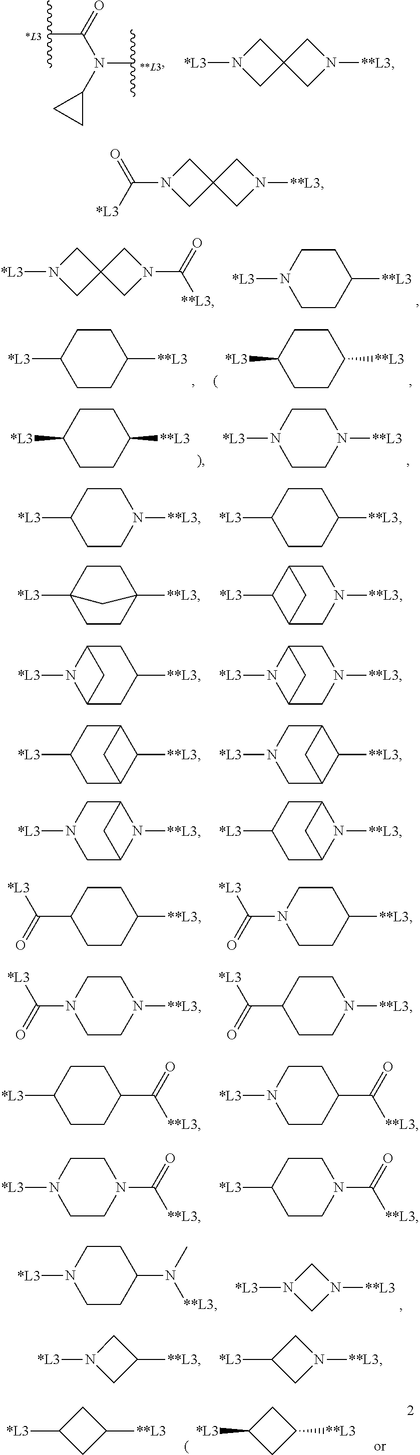

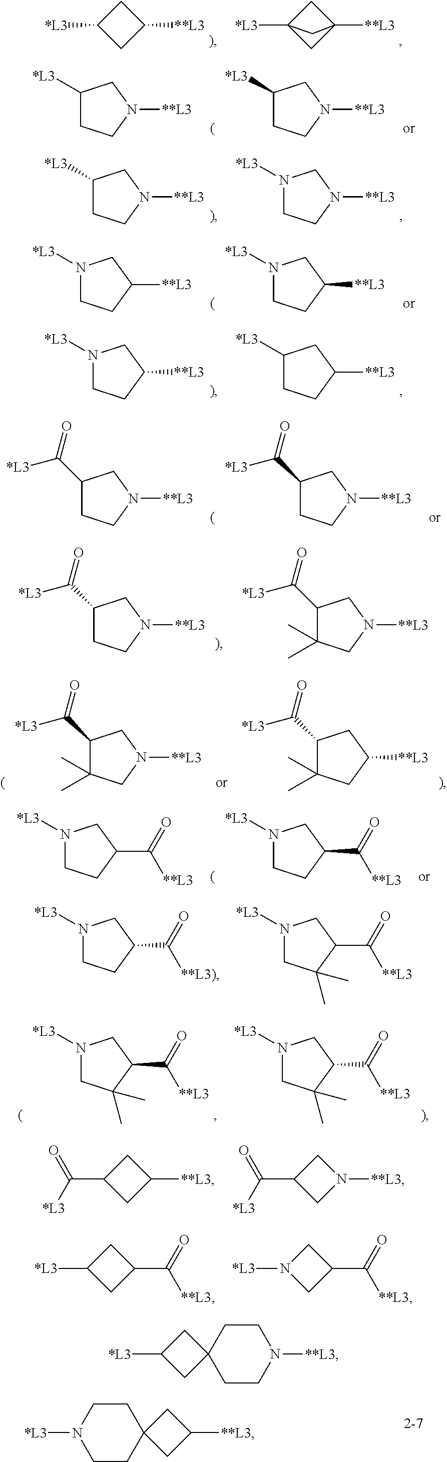

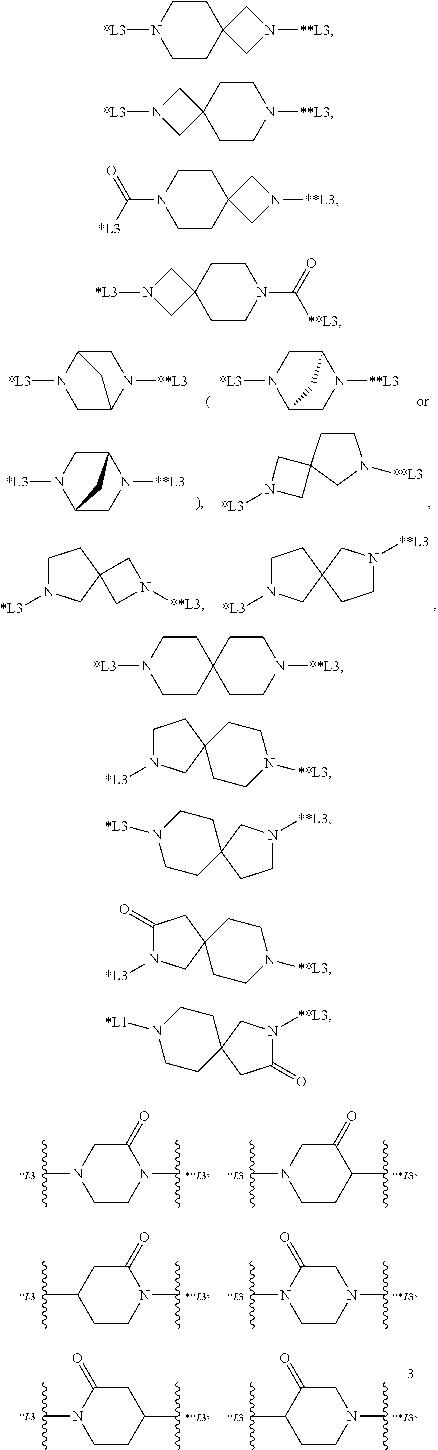

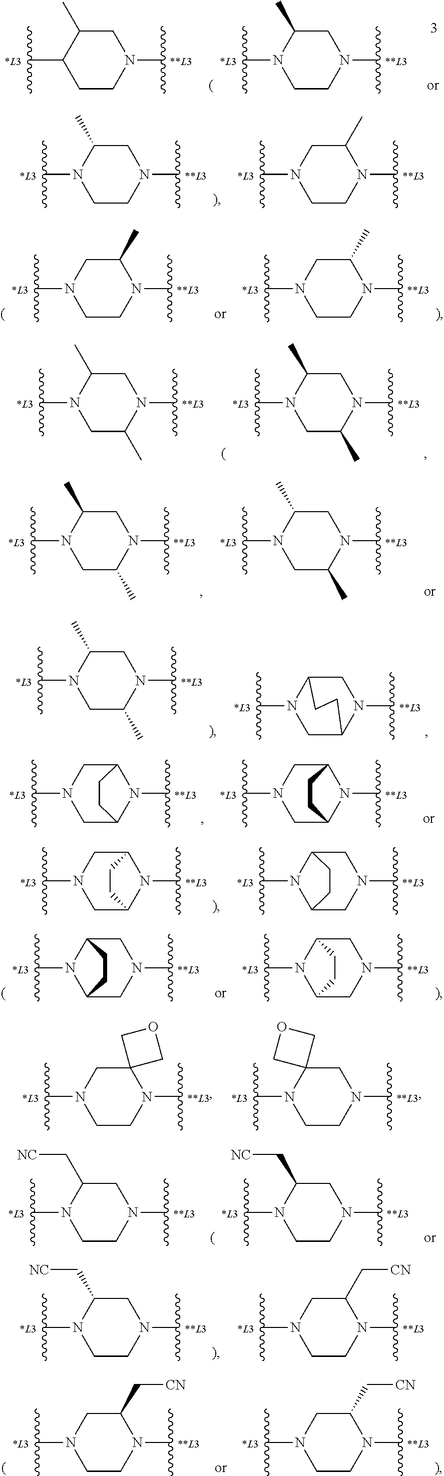

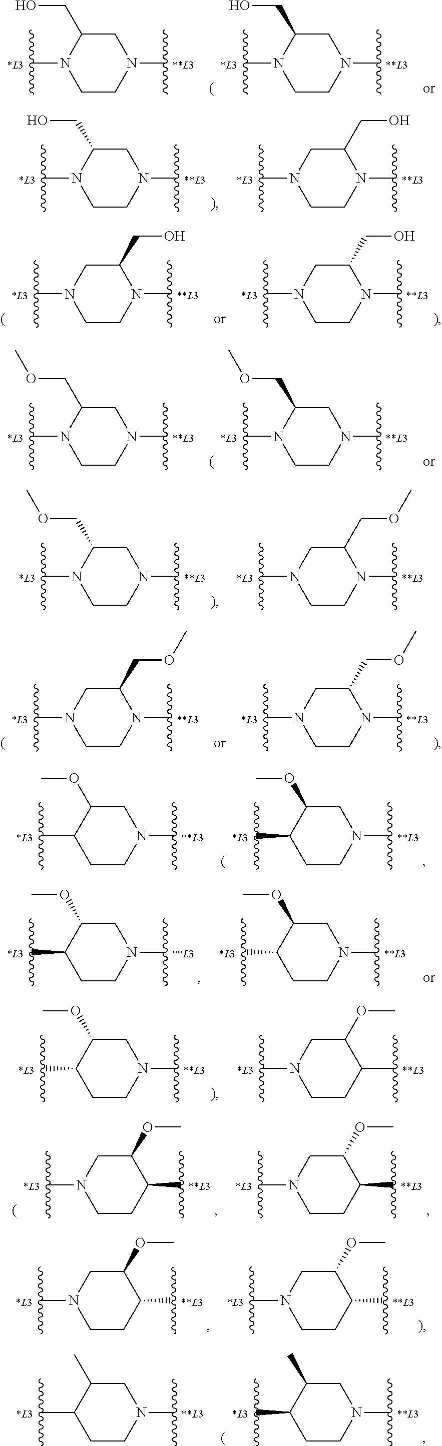

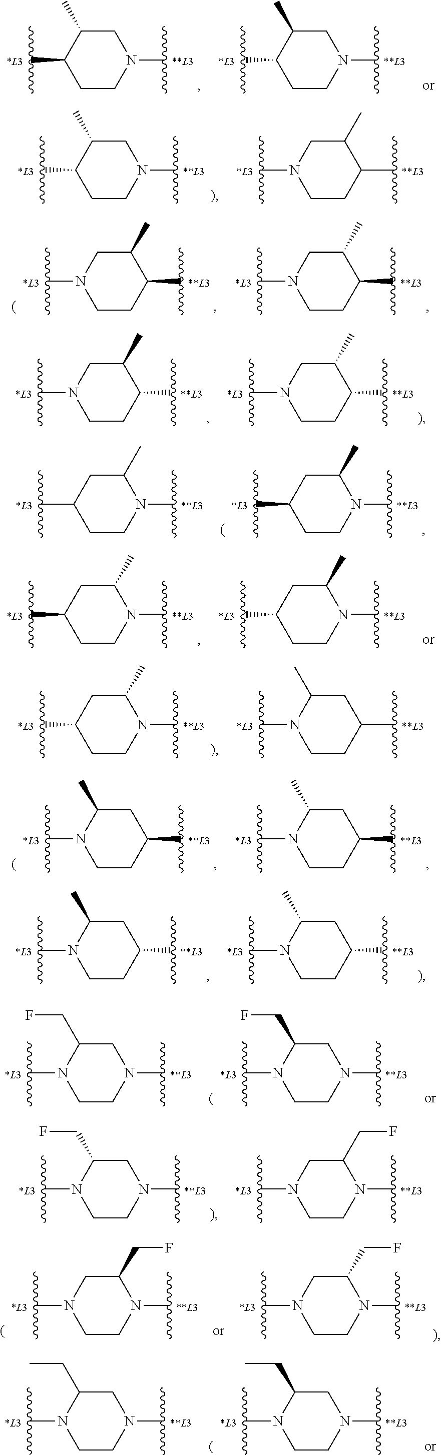

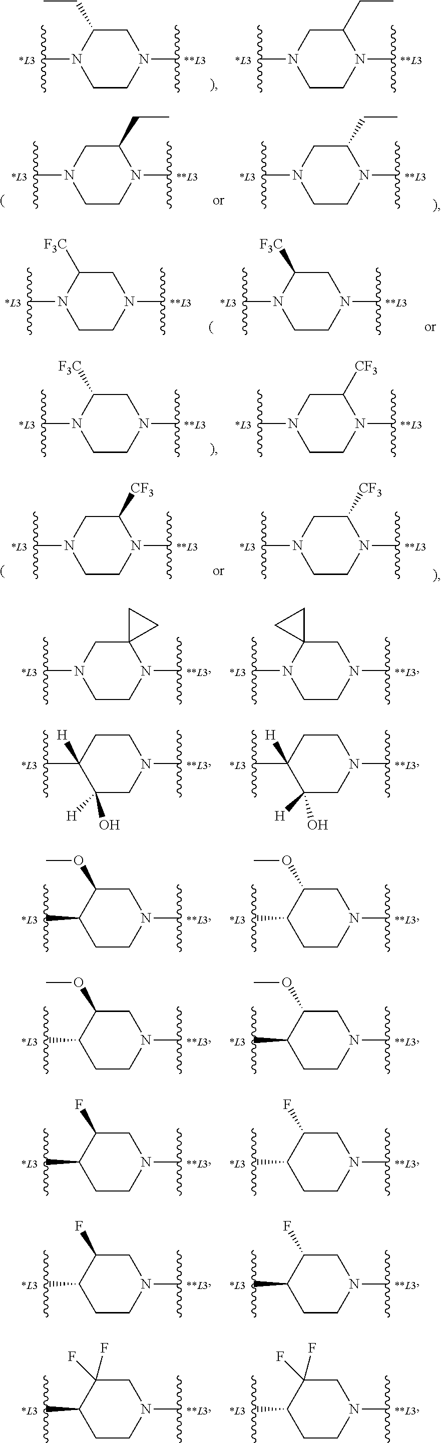

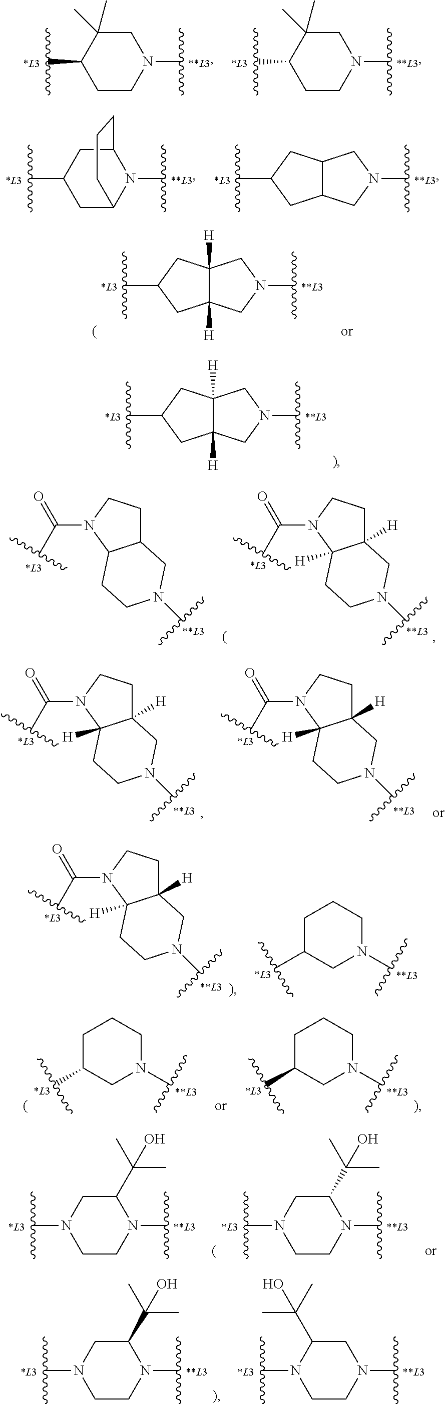

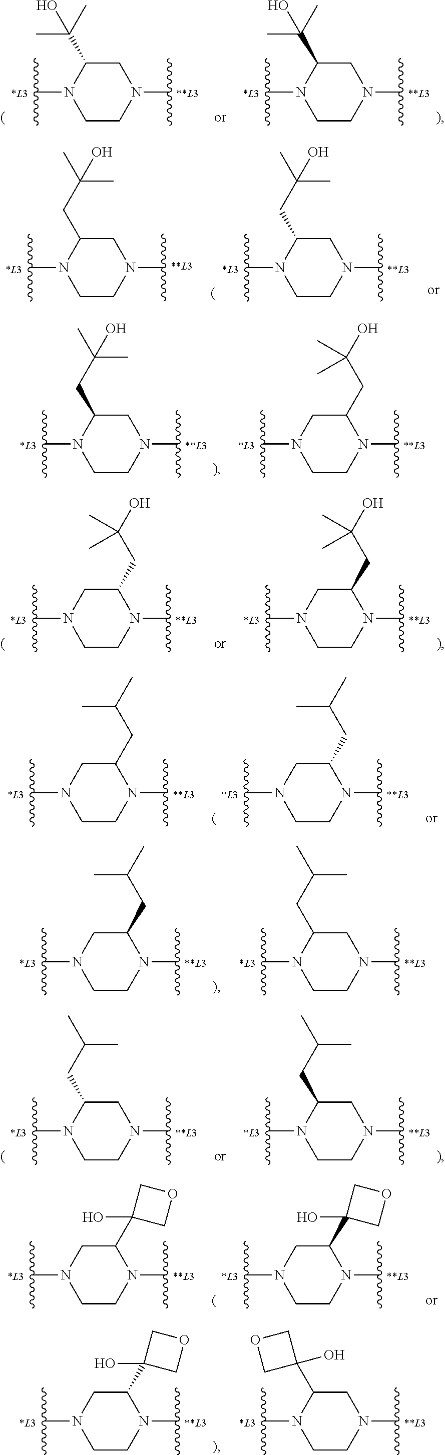

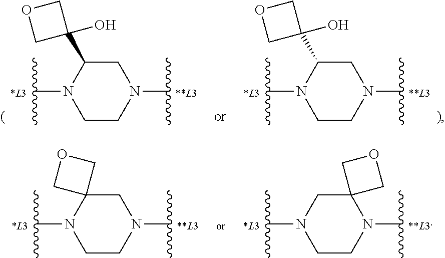

Aspect 25. The compound of any one of the preceding Aspects, wherein

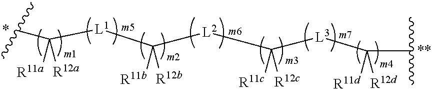

moiety is selected from

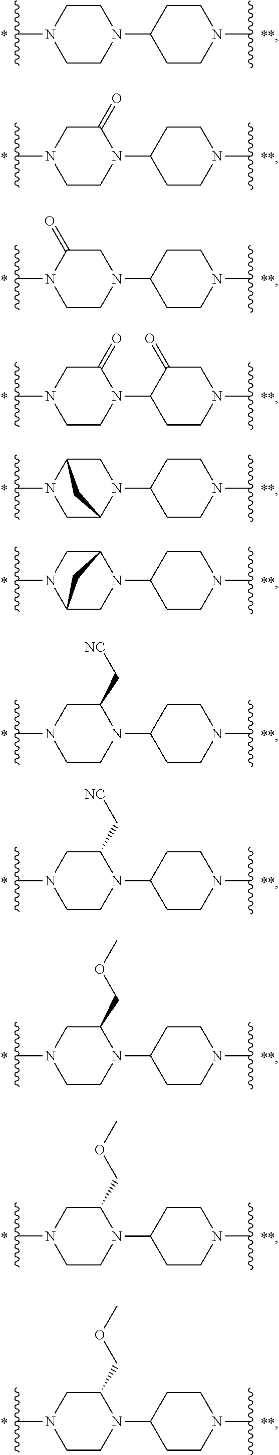

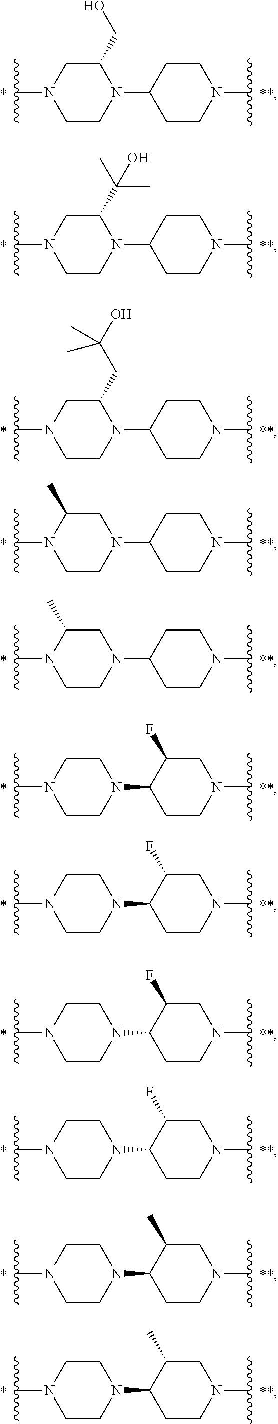

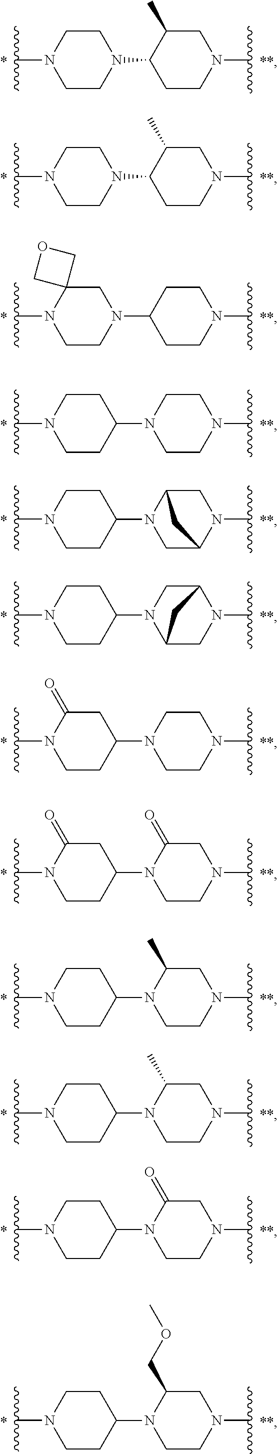

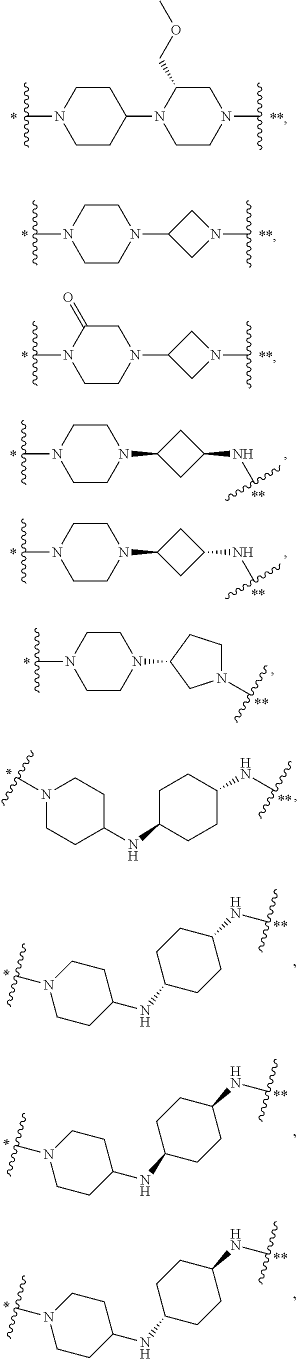

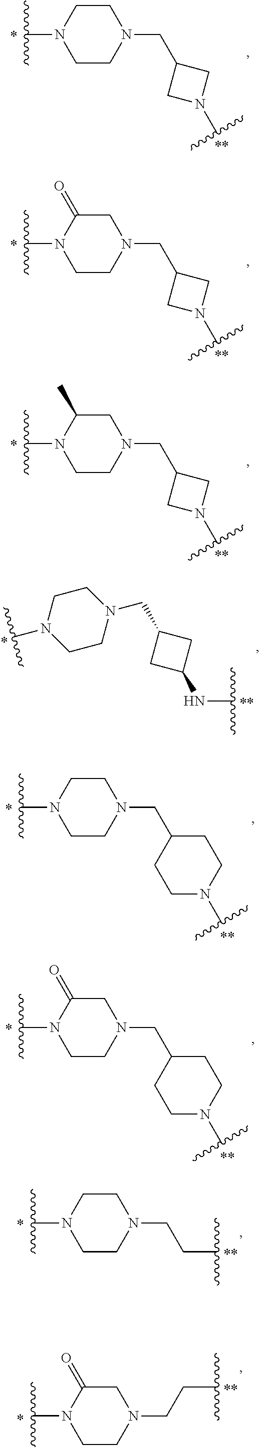

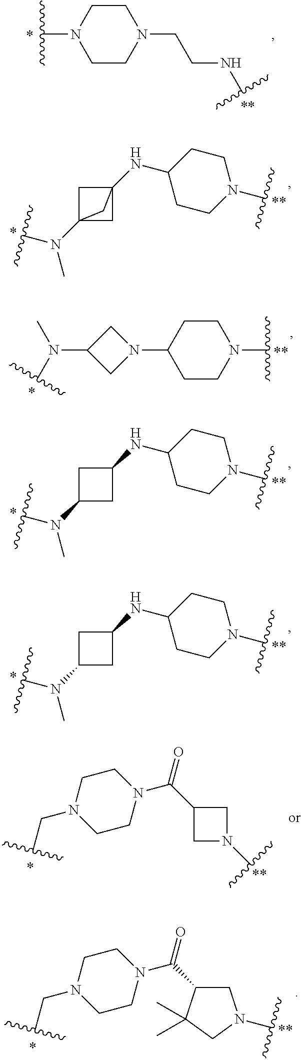

Aspect 26. The compound of any one of the preceding Aspects, wherein

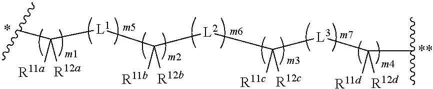

moiety is selected from

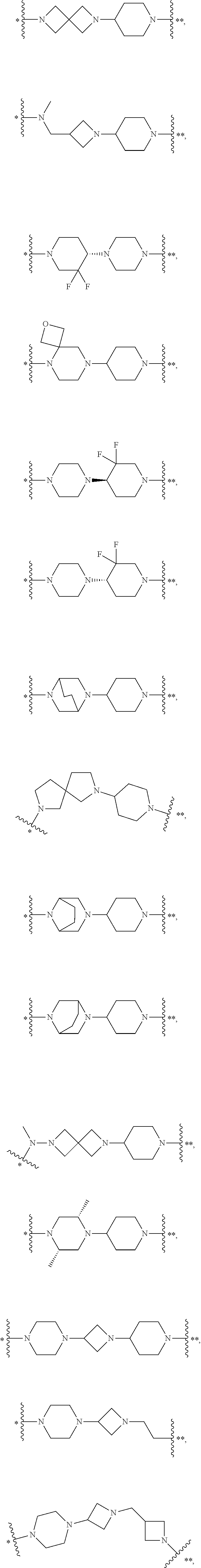

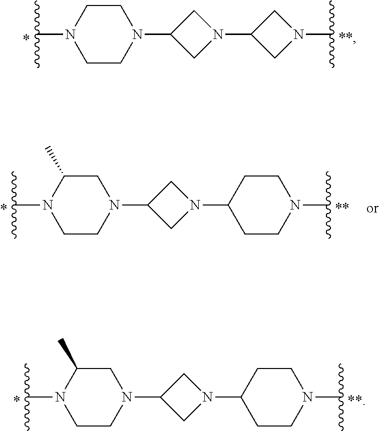 [SMILES: *N1CCN(C2CN(C3CN(*)C3)C2)CC1 ; *N1CCC(N2CC(N3CCN(*)[C@H](C)C3)C2)CC1 ; *N1CCC(N2CC(N3CCN(*)[C@@H](C)C3)C2)CC1]

Aspect 27. The compound of any one of the preceding Aspects, wherein X⁷ is independently selected from —CR^{a}, or N;
R^{a} is independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, X⁷ is independently selected from —CH, —C(CH₃), or N; preferably X⁷ is independently selected from —CH.

Aspect 28. The compound of any one of the preceding Aspects, wherein X⁸ is independently selected from —NR^{a}—, —O—, —S— and —CR^{a}R^{b}—;
at each occurrence, R and Rb are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, X⁸ is independently selected from —NH— and —CH₂—; preferably X⁸ is independently selected from —CH₂—.

Aspect 29. The compound of any one of the preceding Aspects, wherein [symbol] is selected from

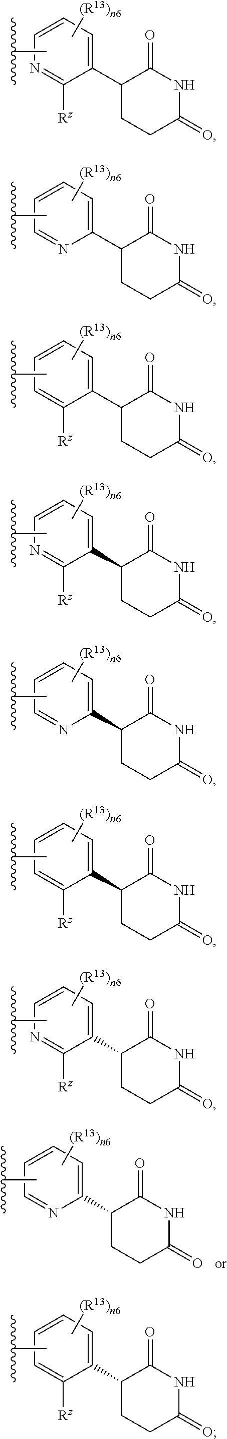

preferably [symbol] is selected from

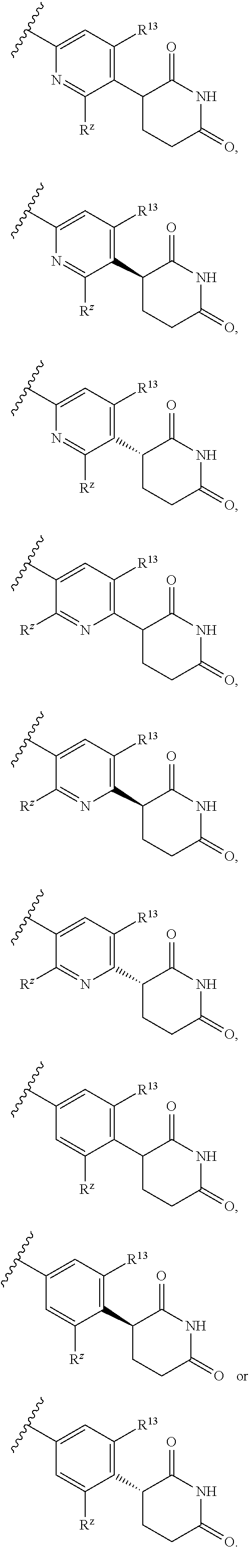

Aspect 30. The compound of any one of the preceding Aspects, wherein at most one of Z¹ and Z² is N; preferably Z¹ and Z² are each independently CR^{z}.

Aspect 31. The compound of any one of the preceding Aspects, wherein R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl or CN; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one R^{Zc};
R^{Za} and R^{Zb} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R^{z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃.

Aspect 32. The compound of any one of the preceding Aspects, wherein R¹³ is each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₂aryl, 5- to 12-membered heteroaryl, oxo, —CN, —OR^{13c}, —SO₂R³, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₂aryl, or 5- to 12-membered heteroaryl; preferably, R¹³ is each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.

Aspect 33. The compound of any one of the preceding Aspects, wherein [symbol] is

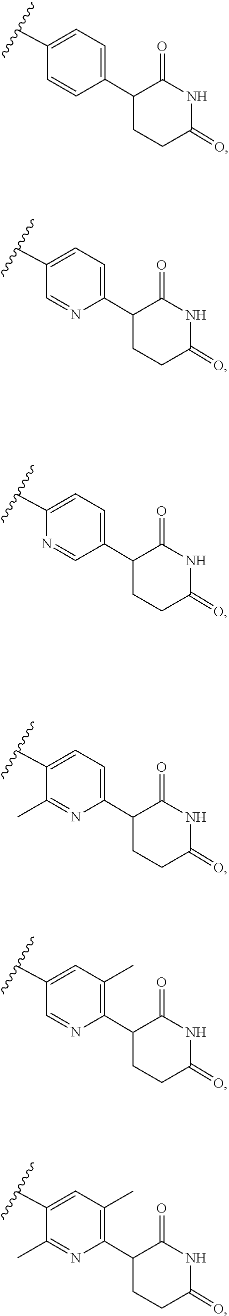 [SMILES: *c1ccc(C2CCC(=O)NC2=O)cc1 ; *c1ccc(C2CCC(=O)NC2=O)nc1 ; *c1ccc(C2CCC(=O)NC2=O)cn1 ; *c1ccc(C2CCC(=O)NC2=O)nc1C ; *c1cnc(C2CCC(=O)NC2=O)c(C)c1 ; *c1cc(C)c(C2CCC(=O)NC2=O)nc1C]

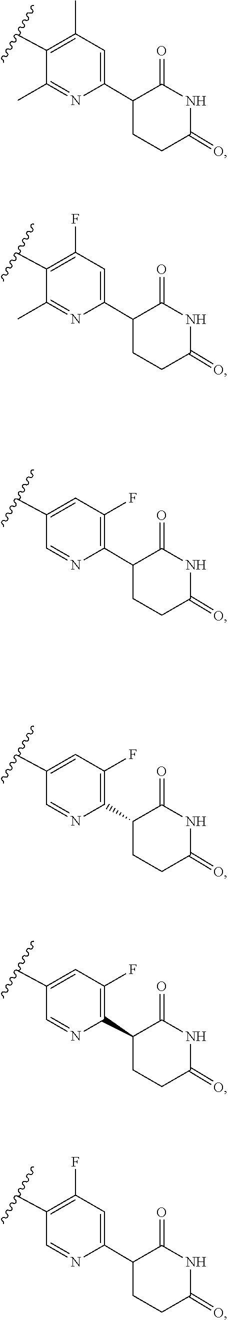 [SMILES: *c1c(C)cc(C2CCC(=O)NC2=O)nc1C ; *c1c(F)cc(C2CCC(=O)NC2=O)nc1C ; *c1cnc(C2CCC(=O)NC2=O)c(F)c1 ; *c1cnc([C@H]2CCC(=O)NC2=O)c(F)c1 ; *c1cnc([C@@H]2CCC(=O)NC2=O)c(F)c1 ; *c1cnc(C2CCC(=O)NC2=O)cc1F]

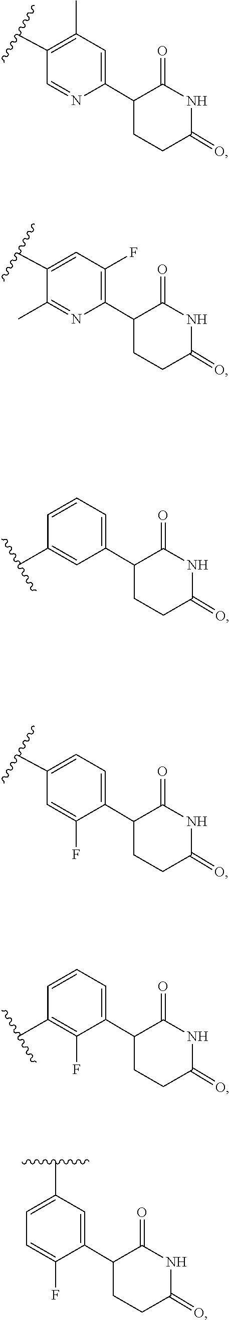 [SMILES: *c1cnc(C2CCC(=O)NC2=O)cc1C ; *c1cc(F)c(C2CCC(=O)NC2=O)nc1C ; *c1cccc(C2CCC(=O)NC2=O)c1 ; *c1ccc(C2CCC(=O)NC2=O)c(F)c1 ; *c1cccc(C2CCC(=O)NC2=O)c1F ; *c1ccc(F)c(C2CCC(=O)NC2=O)c1]

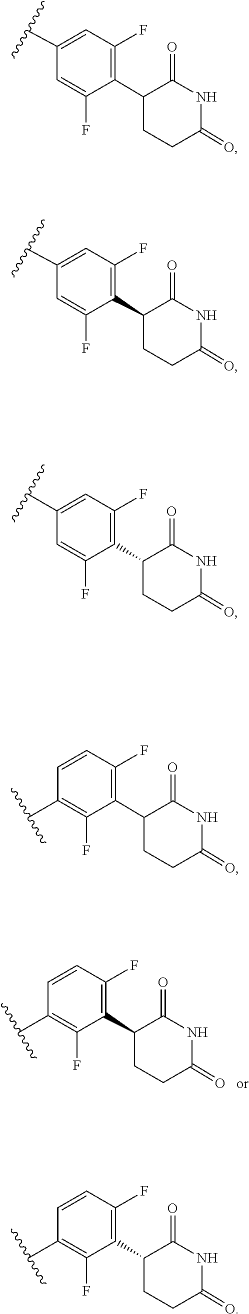

Aspect 34. The compound of any one of the preceding Aspects, wherein [symbol] is

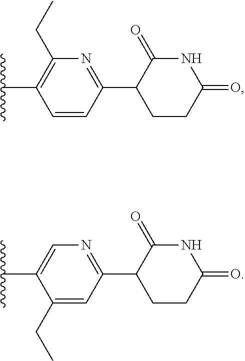 [SMILES: *c1ccc(C2CCC(=O)NC2=O)nc1CC ; *c1cnc(C2CCC(=O)NC2=O)cc1CC]

Aspect 35. The compound of any one of the preceding Aspects is selected from

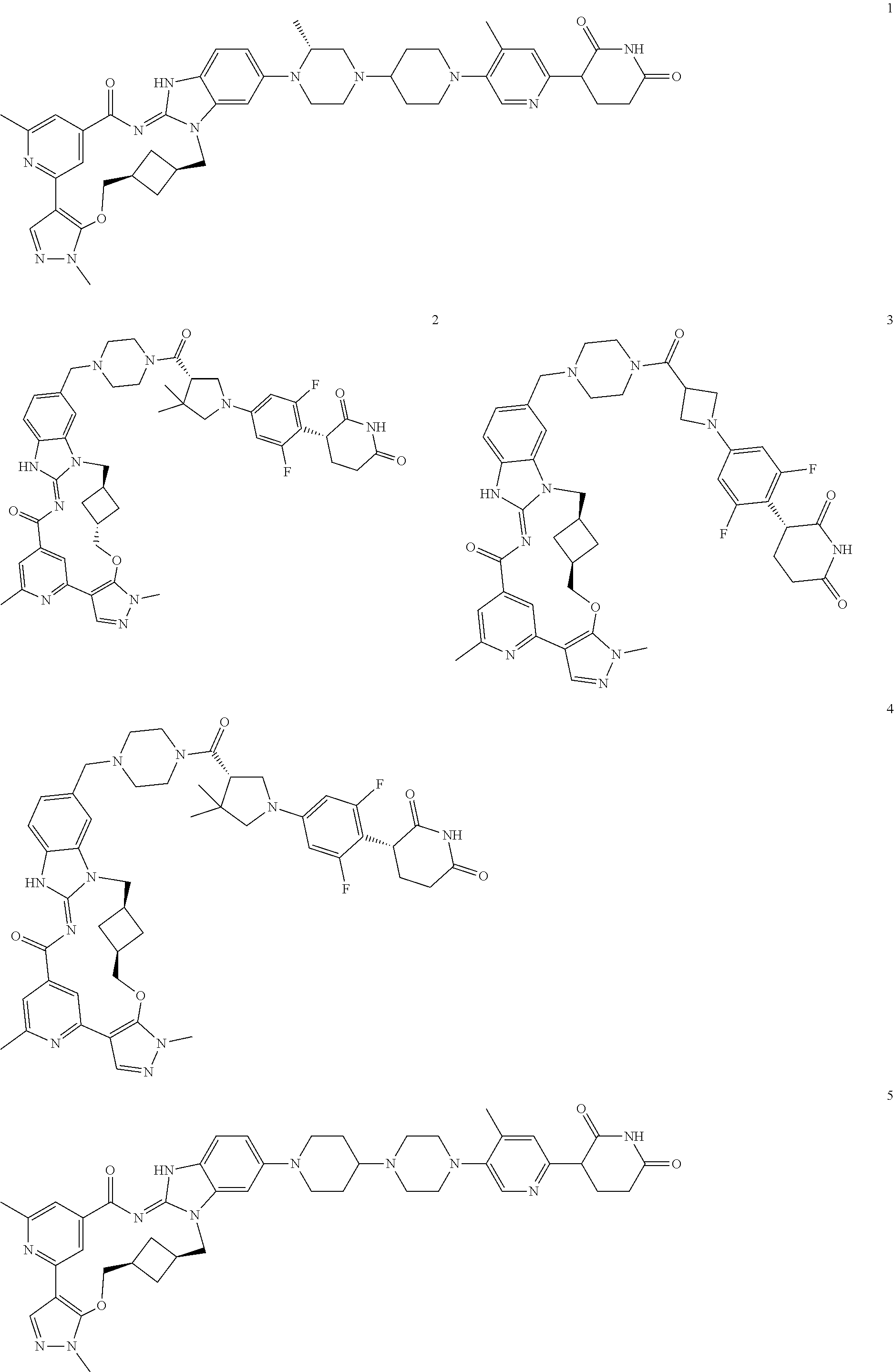

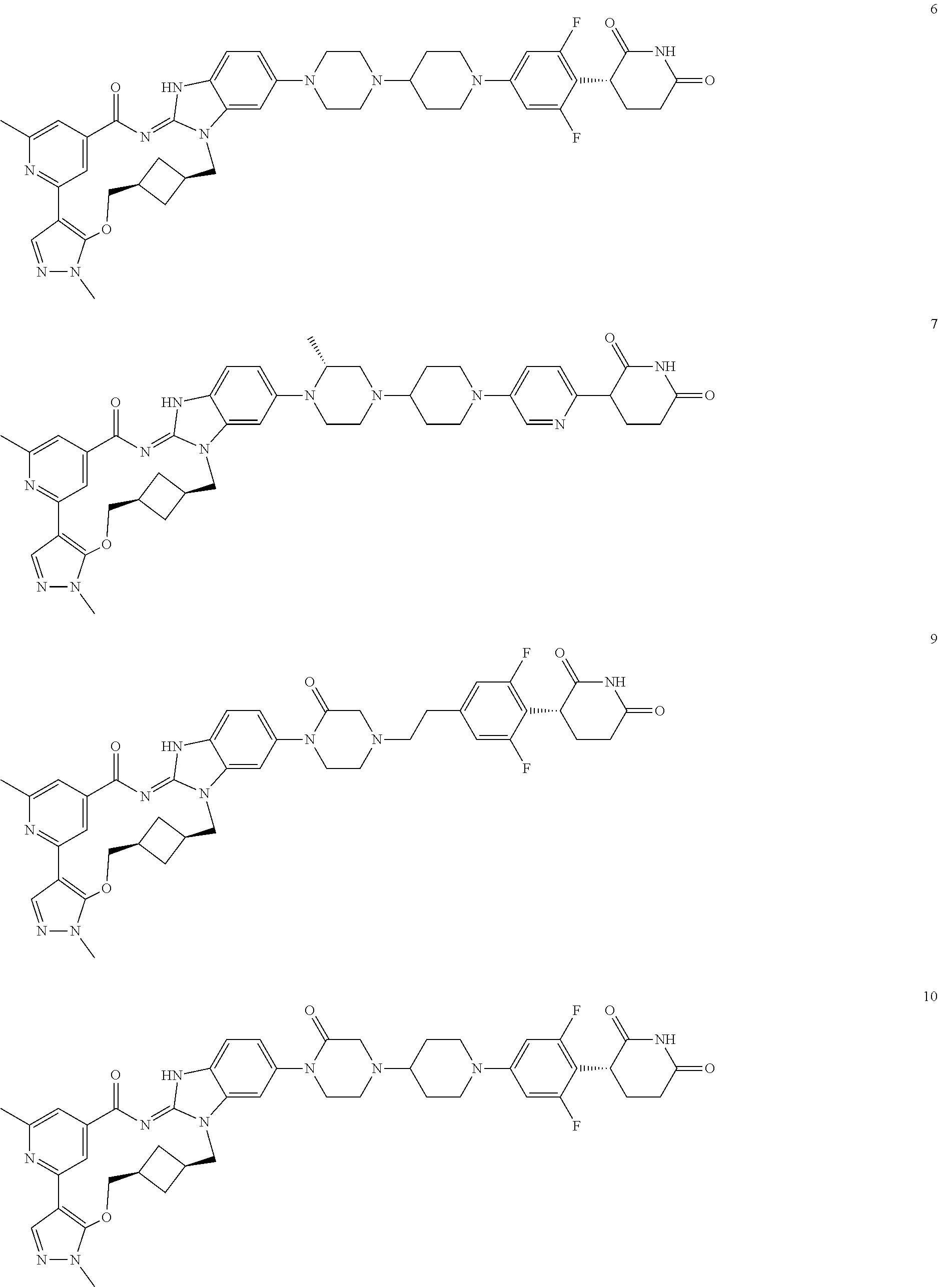 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C[C@H]4C)cc23)C1 ; Cc1cc2cc(n1)-c1cnn(C)c1OC[C@H]1C[C@@H](CN3/C(=N/C2=O)Nc2ccc(N4CCN(CCc5cc(F)c([C@H]6CCC(=O)NC6=O)c(F)c5)CC4=O)cc23)C1 ; Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)CC4=O)cc23)C1]

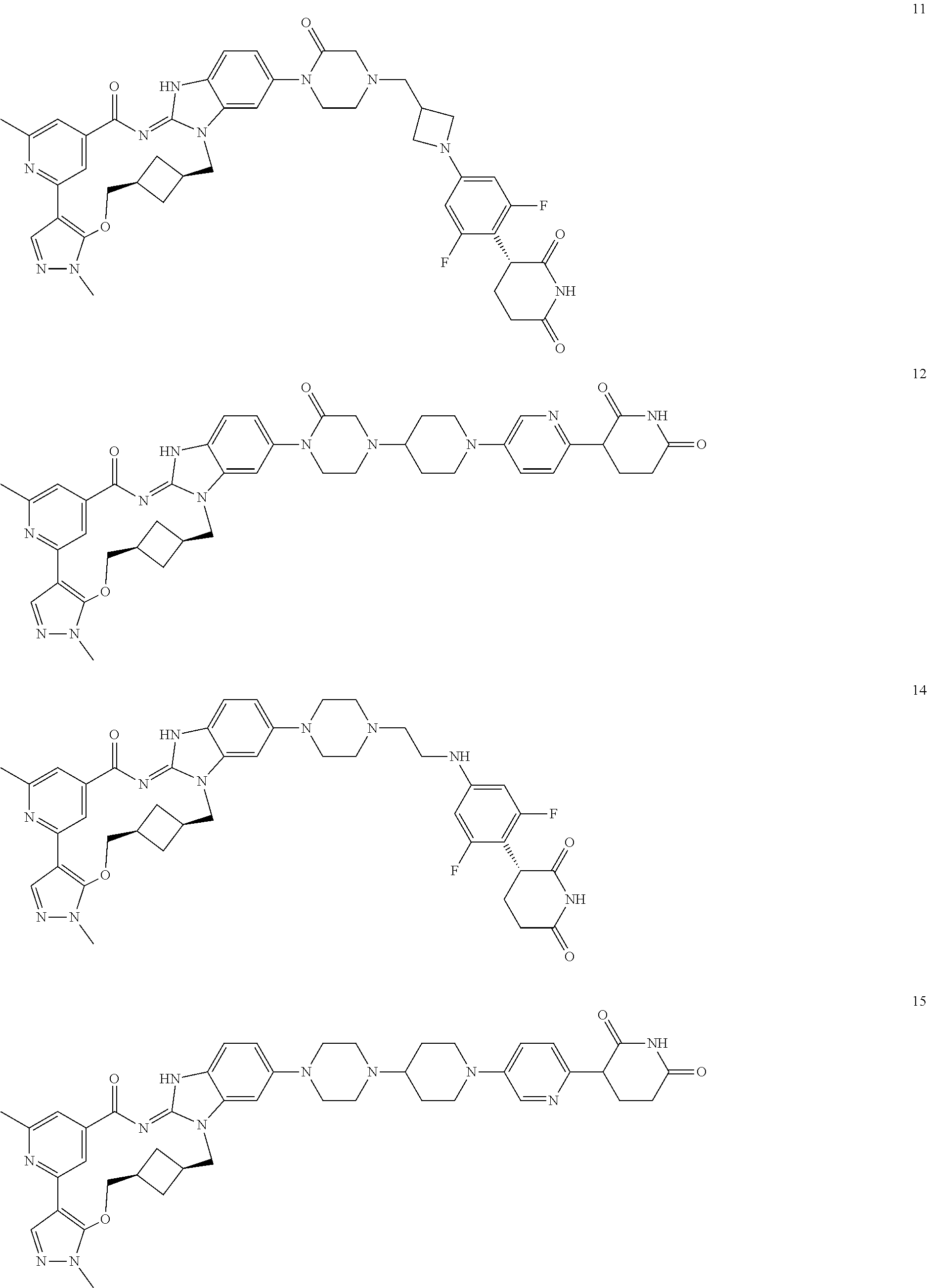 [SMILES: Cc1cc2cc(n1)-c1cnn(C)c1OC[C@H]1C[C@@H](CN3/C(=N/C2=O)Nc2ccc(N4CCN(CC5CN(c6cc(F)c([C@H]7CCC(=O)NC7=O)c(F)c6)C5)CC4=O)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)CC4=O)cc23)C1 ; Cc1cc2cc(n1)-c1cnn(C)c1OC[C@H]1C[C@@H](CN3/C(=N/C2=O)Nc2ccc(N4CCN(CCNc5cc(F)c([C@H]6CCC(=O)NC6=O)c(F)c5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)CC4)cc23)C1]

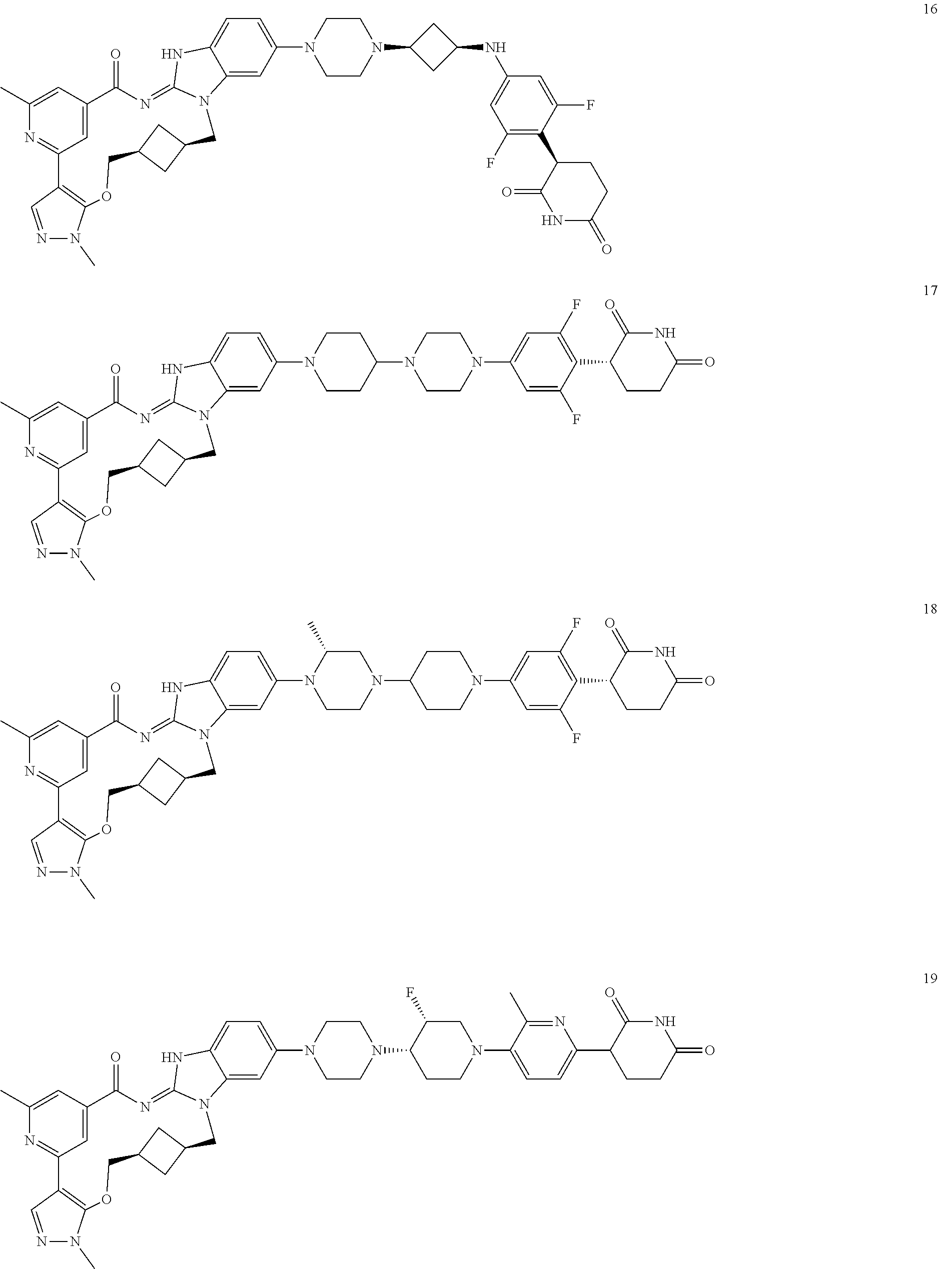

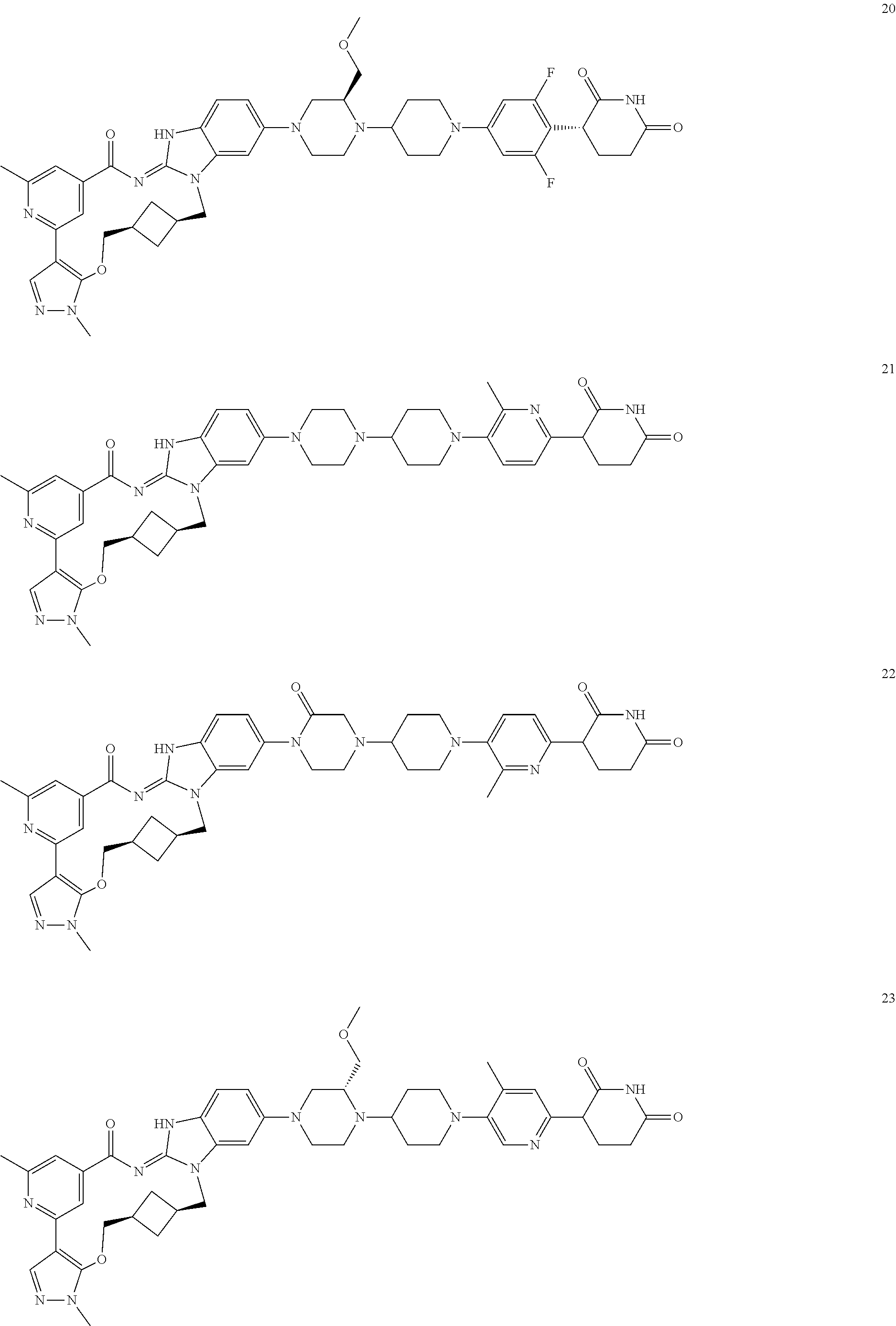 [SMILES: COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2cc(F)c([C@H]3CCC(=O)NC3=O)c(F)c2)CC1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC4=O)cc23)C1 ; COC[C@H]1CN(c2ccc3c(c2)N2C[C@H]4C[C@@H](COc5c(cnn5C)-c5cc(cc(C)n5)C(=O)/N=C/2N3)C4)CCN1C1CCN(c2cnc(C3CCC(=O)NC3=O)cc2C)CC1]

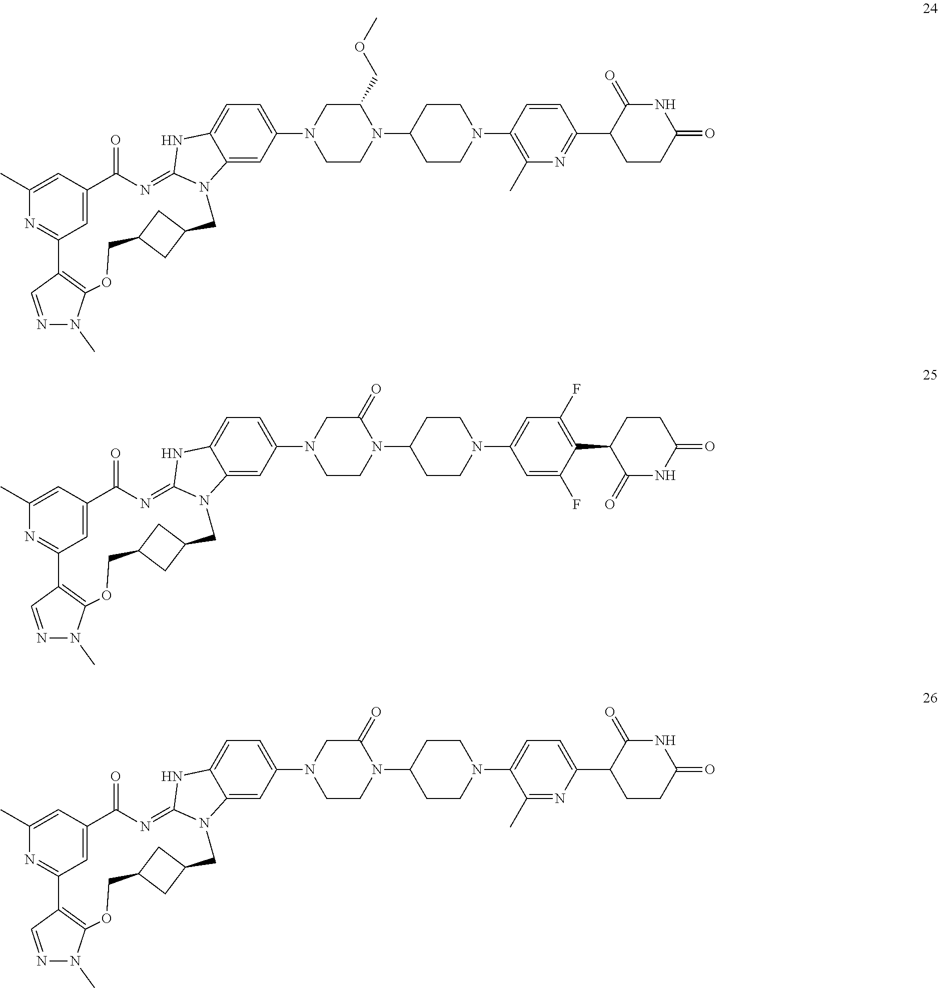 [SMILES: COC[C@H]1CN(c2ccc3c(c2)N2C[C@H]4C[C@@H](COc5c(cnn5C)-c5cc(cc(C)n5)C(=O)/N=C/2N3)C4)CCN1C1CCN(c2ccc(C3CCC(=O)NC3=O)nc2C)CC1 ; Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)C(=O)C4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C(=O)C4)cc23)C1]

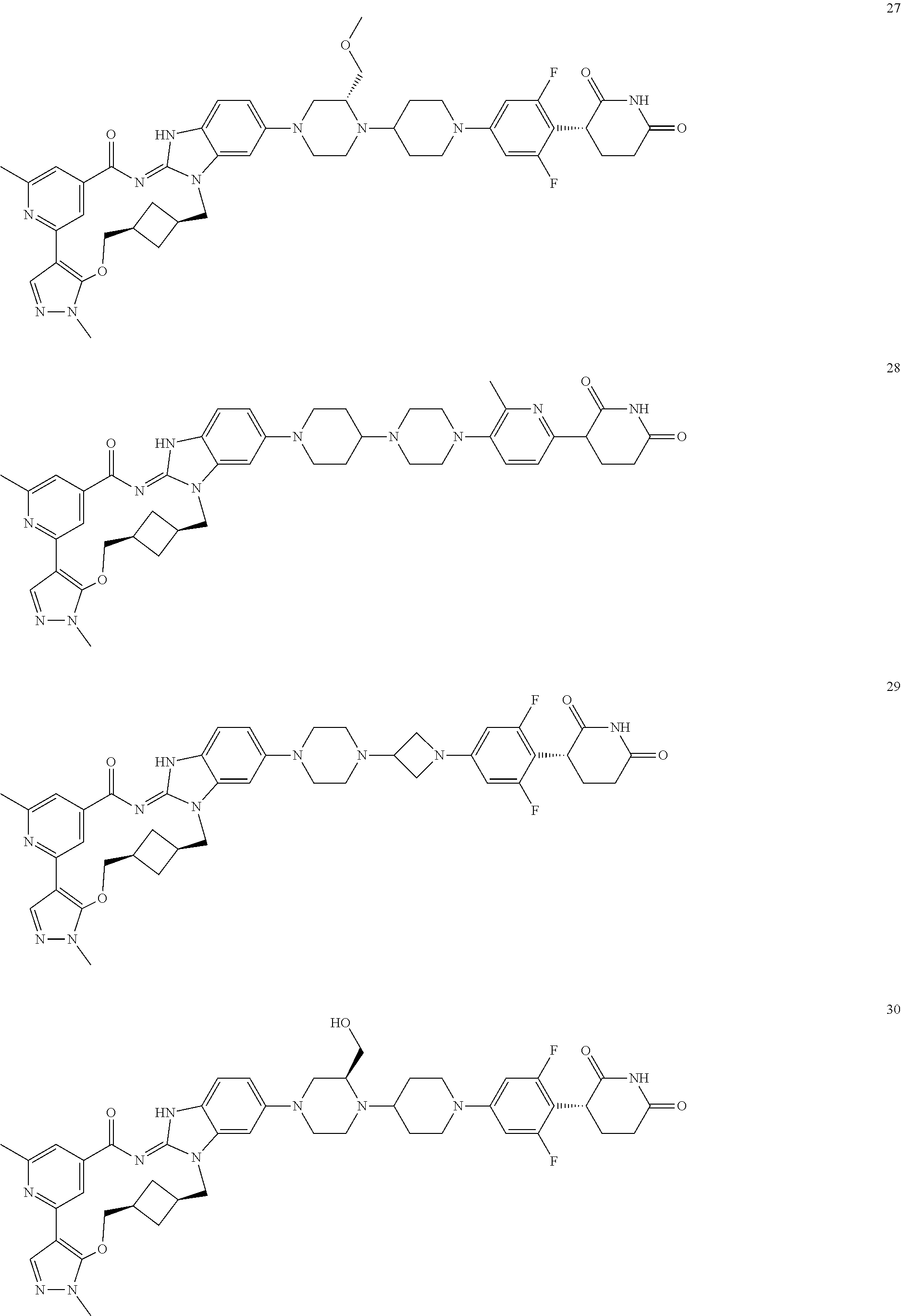

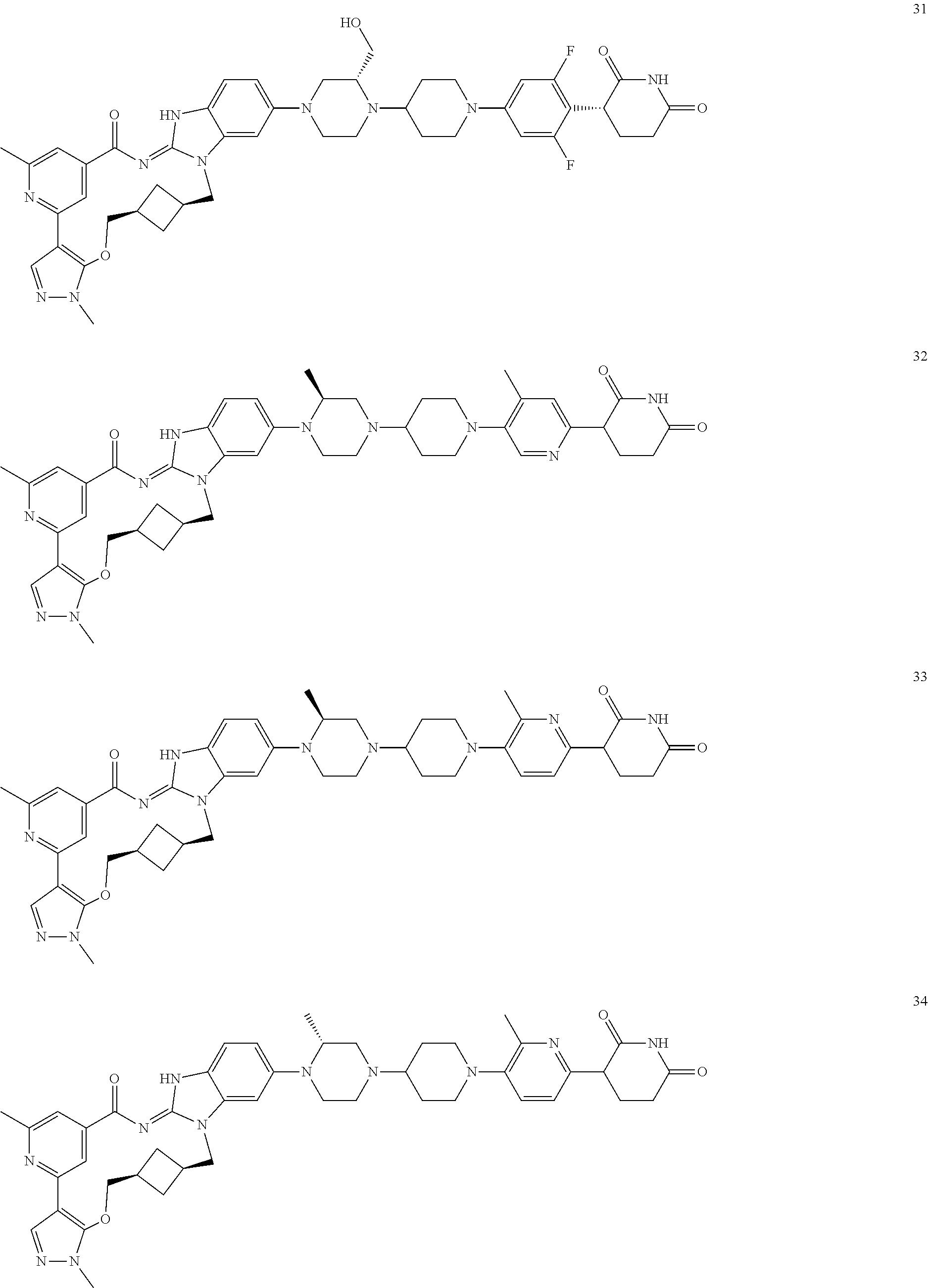 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)[C@@H](CO)C4)cc23)C1 ; Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)C[C@@H]4C)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C[C@@H]4C)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C[C@H]4C)cc23)C1]

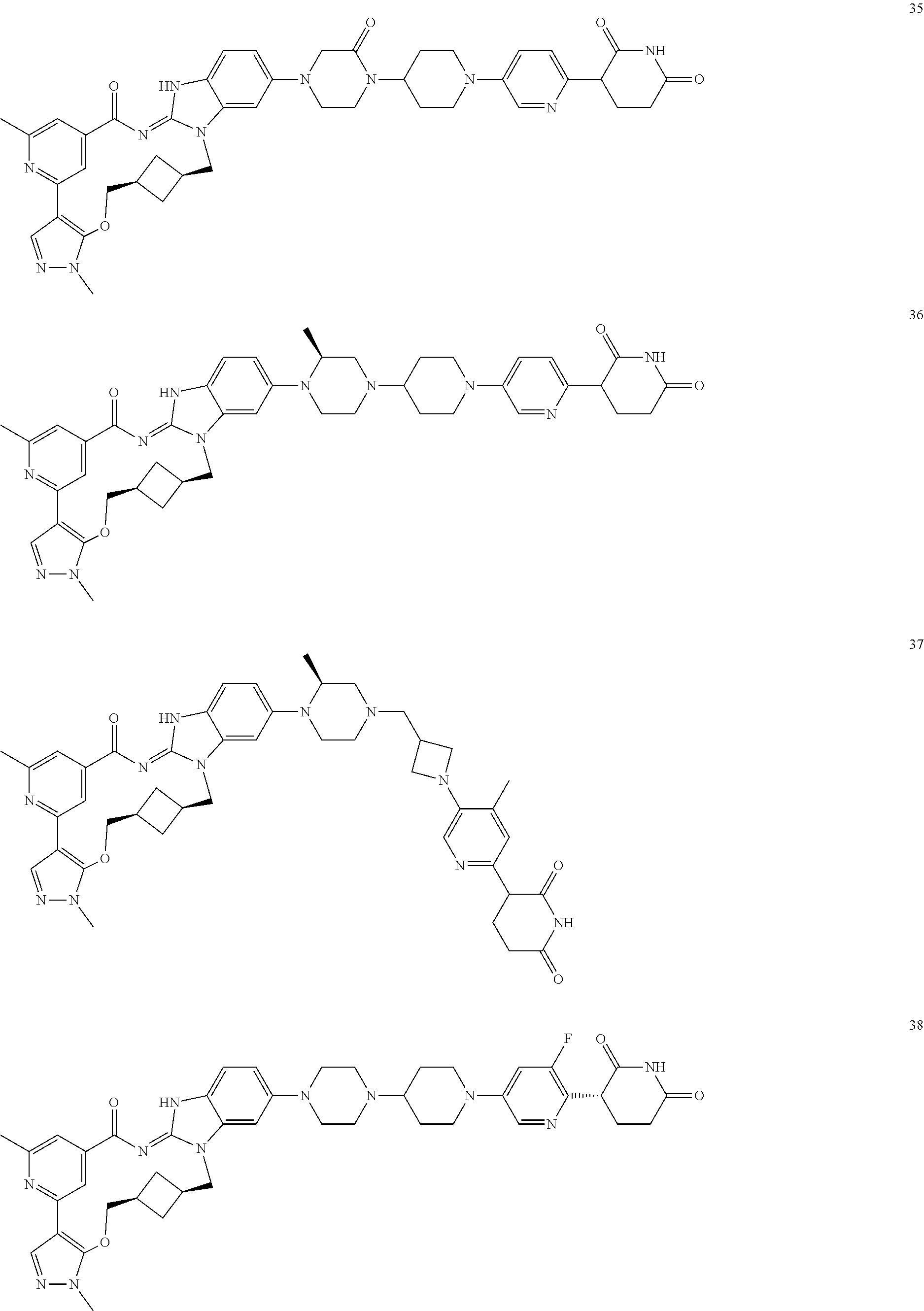 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C(=O)C4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C[C@@H]4C)cc23)C1 ; Cc1cc2cc(n1)-c1cnn(C)c1OC[C@H]1C[C@@H](CN3/C(=N/C2=O)Nc2ccc(N4CCN(CC5CN(c6cnc(C7CCC(=O)NC7=O)cc6C)C5)C[C@@H]4C)cc23)C1 ; Cc1cc2cc(n1)-c1cnn(C)c1OC[C@H]1C[C@@H](CN3/C(=N/C2=O)Nc2ccc(N4CCN(C5CCN(c6cnc([C@H]7CCC(=O)NC7=O)c(F)c6)CC5)CC4)cc23)C1]

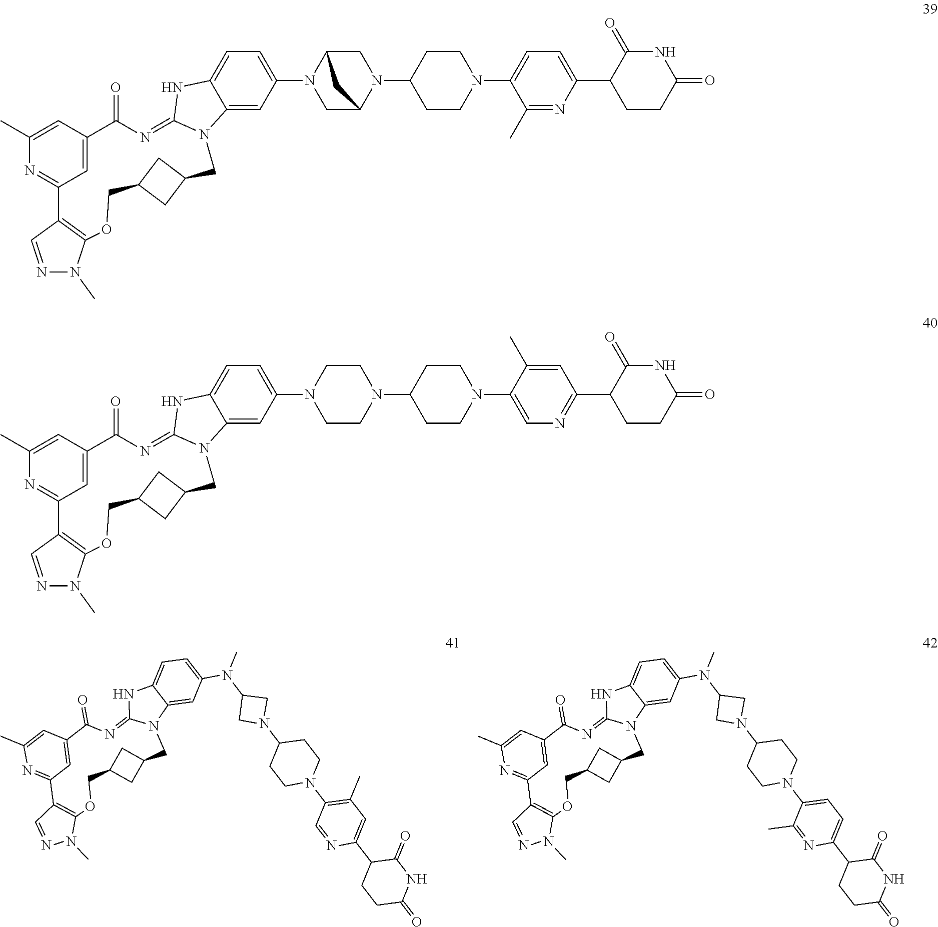 [SMILES: Cc1cc2cc(n1)-c1cnn(C)c1OC[C@H]1C[C@@H](CN3/C(=N/C2=O)Nc2ccc(N4C[C@@H]5C[C@H]4CN5C4CCN(c5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)CC4)cc23)C1 ; Cc1cc2cc(n1)-c1cnn(C)c1OC[C@H]1C[C@@H](CN3/C(=N/C2=O)Nc2ccc(N(C)C4CN(C5CCN(c6cnc(C7CCC(=O)NC7=O)cc6C)CC5)C4)cc23)C1 ; Cc1cc2cc(n1)-c1cnn(C)c1OC[C@H]1C[C@@H](CN3/C(=N/C2=O)Nc2ccc(N(C)C4CN(C5CCN(c6ccc(C7CCC(=O)NC7=O)nc6C)CC5)C4)cc23)C1]

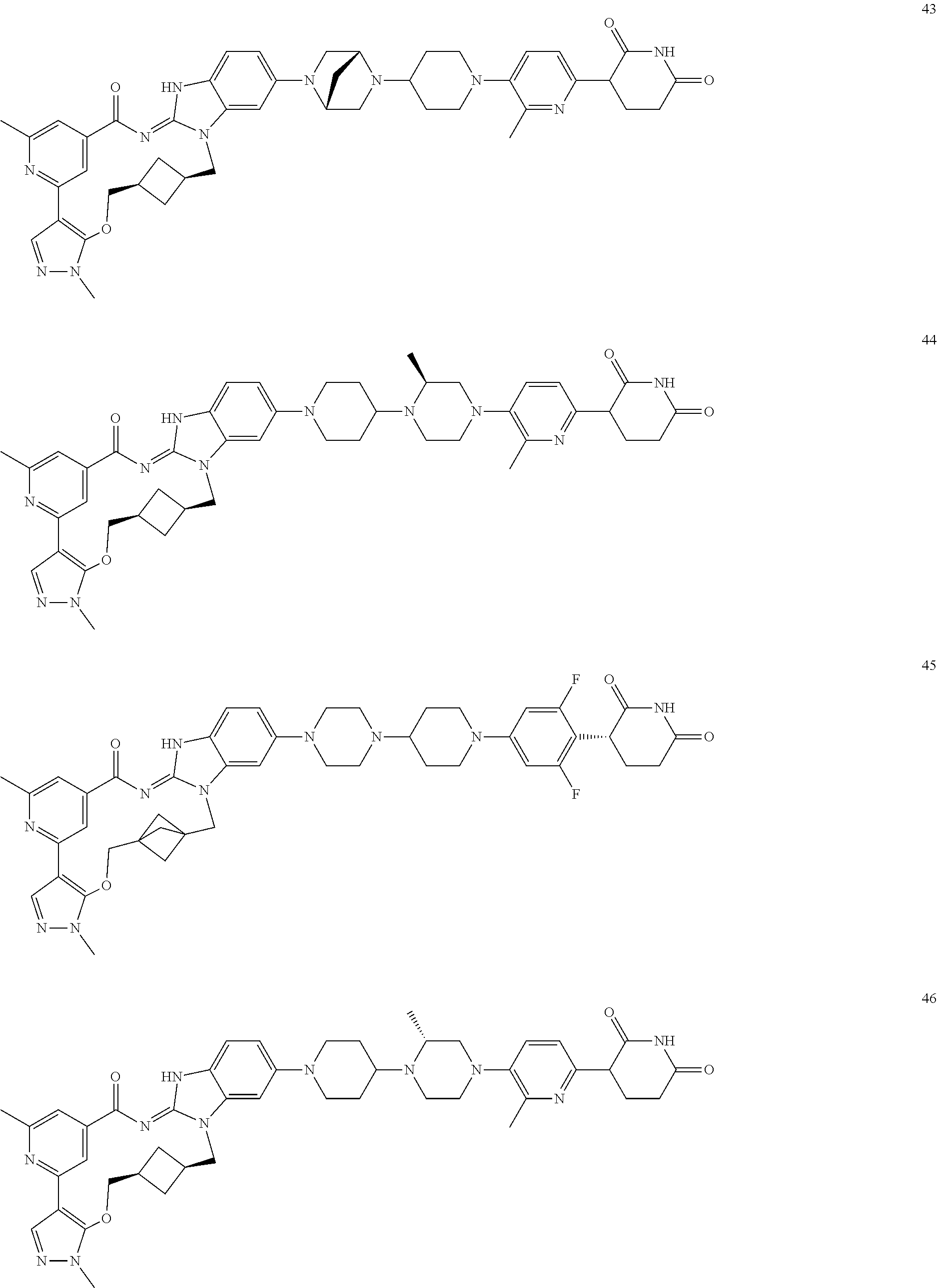 [SMILES: Cc1cc2cc(n1)-c1cnn(C)c1OC[C@H]1C[C@@H](CN3/C(=N/C2=O)Nc2ccc(N4C[C@H]5C[C@@H]4CN5C4CCN(c5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)C[C@@H]5C)CC4)cc23)C1 ; Cc1cc2cc(n1)-c1cnn(C)c1OCC13CC(CN4/C(=N/C2=O)Nc2ccc(N5CCN(C6CCN(c7cc(F)c([C@H]8CCC(=O)NC8=O)c(F)c7)CC6)CC5)cc24)(C1)C3 ; Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)C[C@H]5C)CC4)cc23)C1]

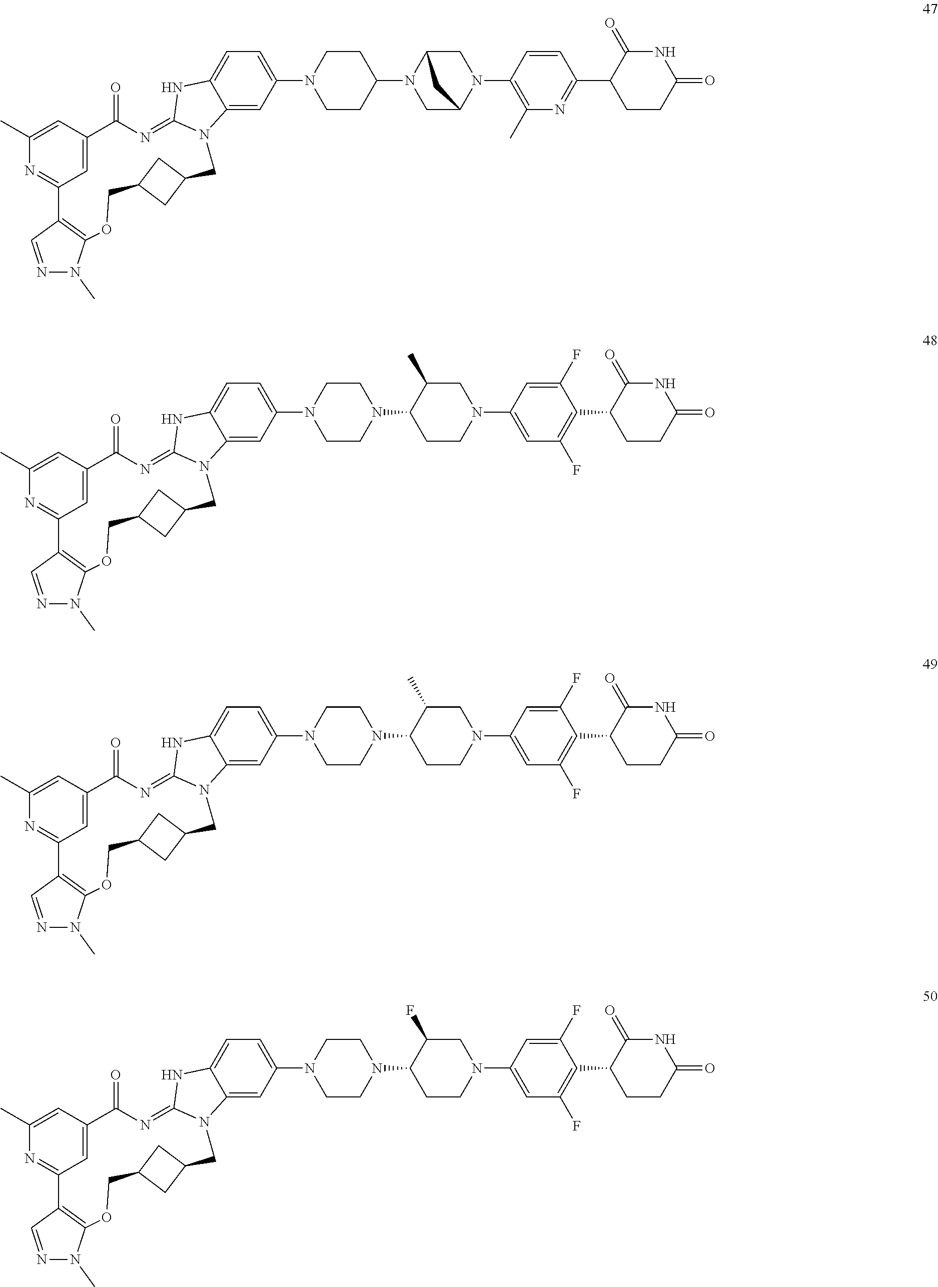

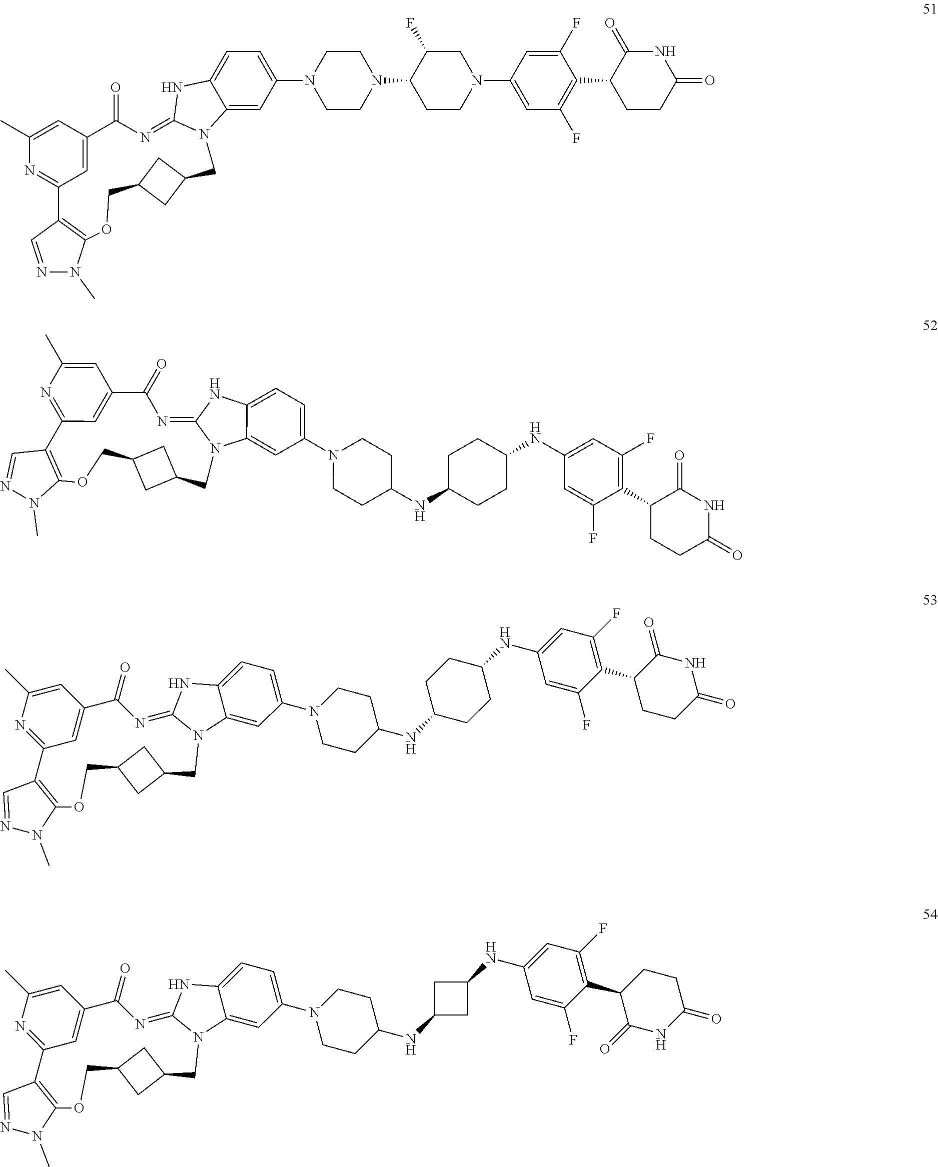

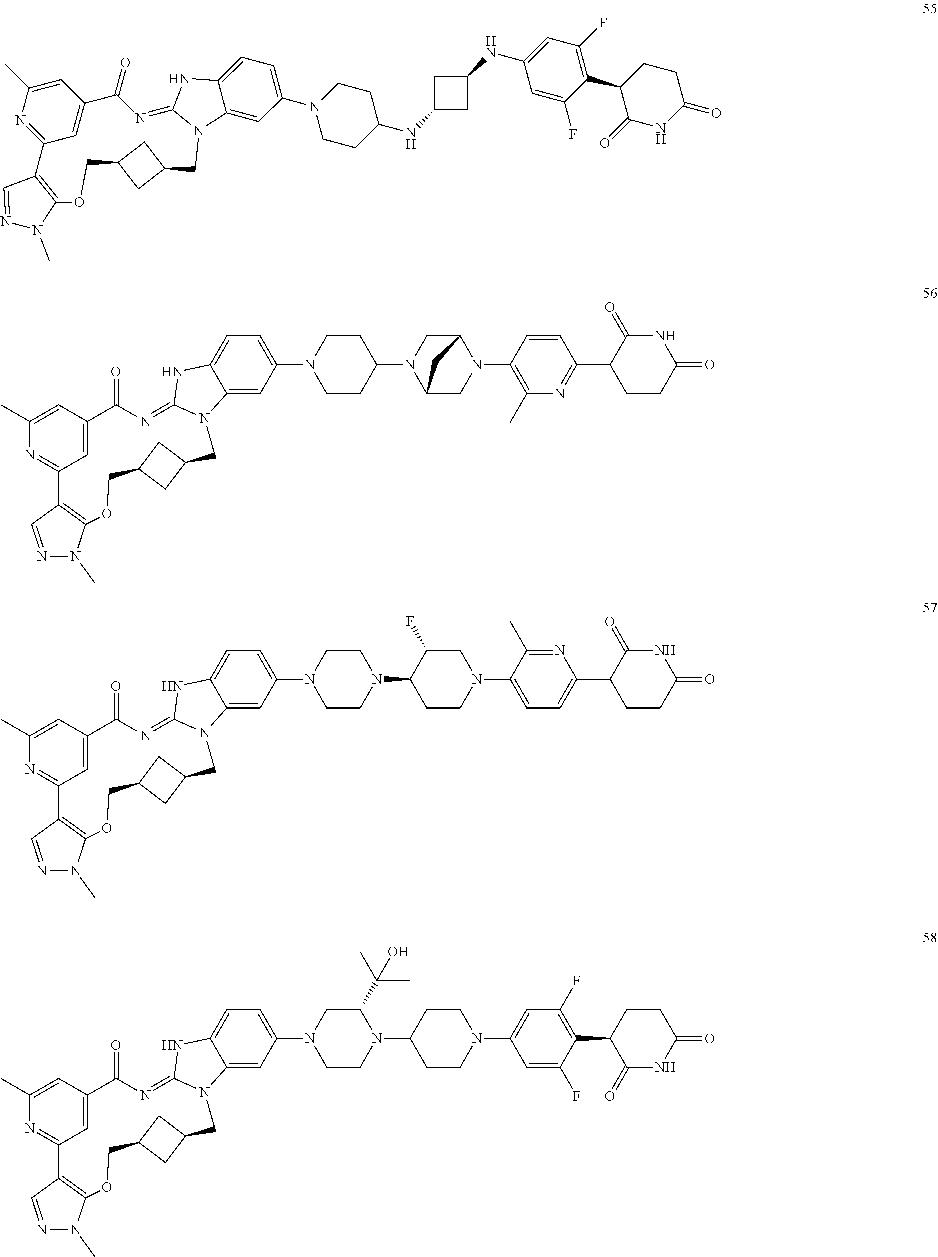

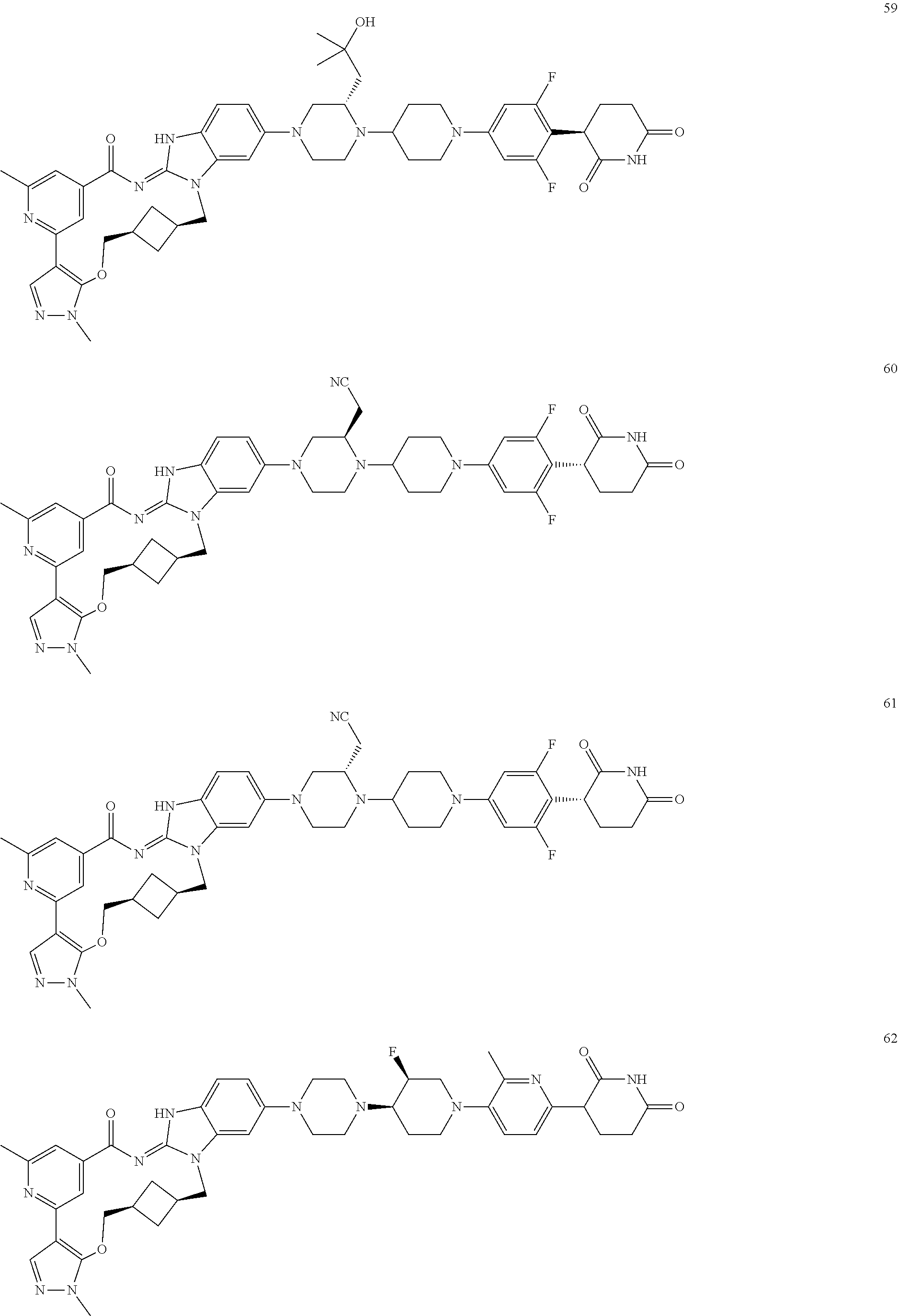

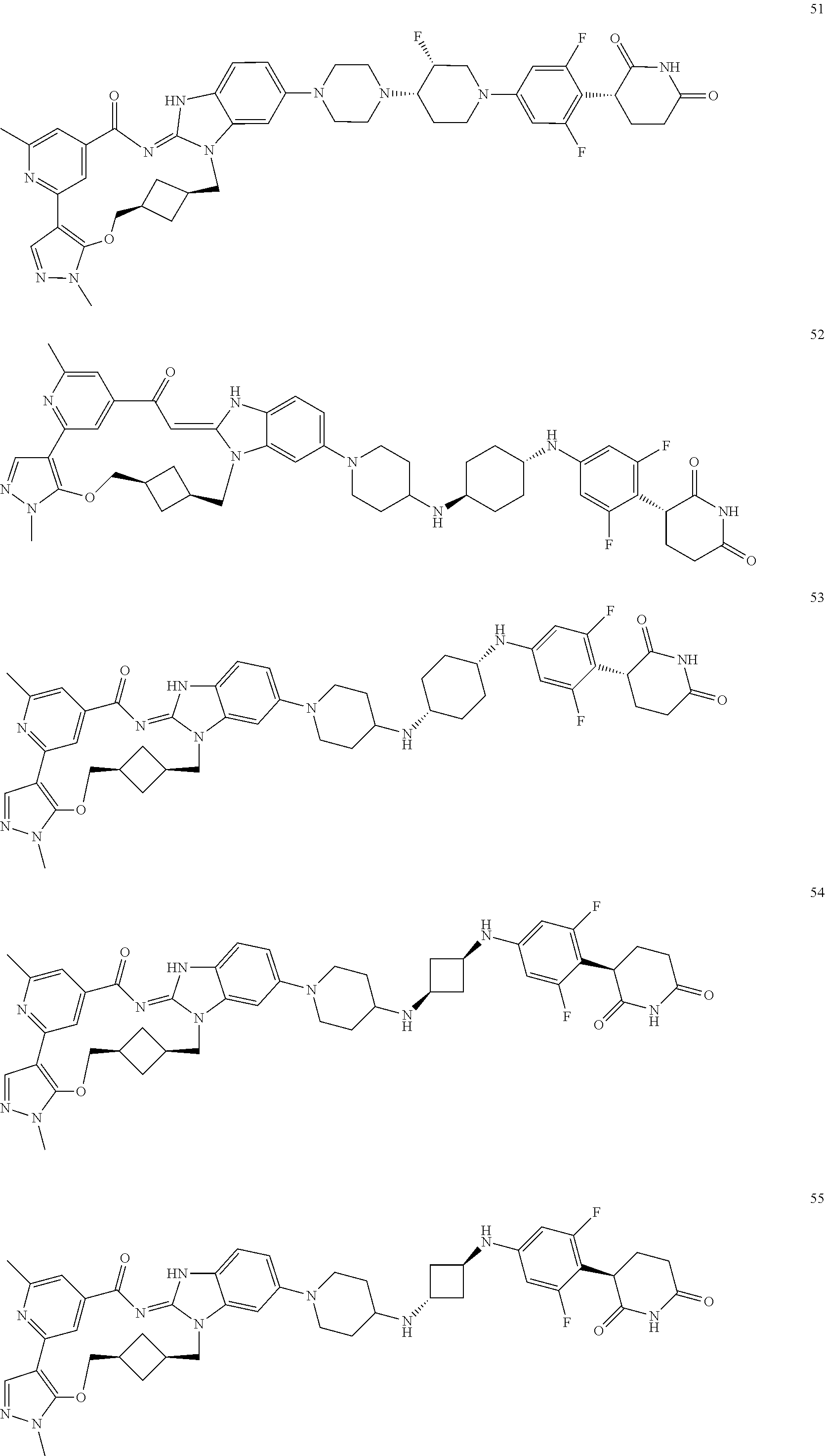

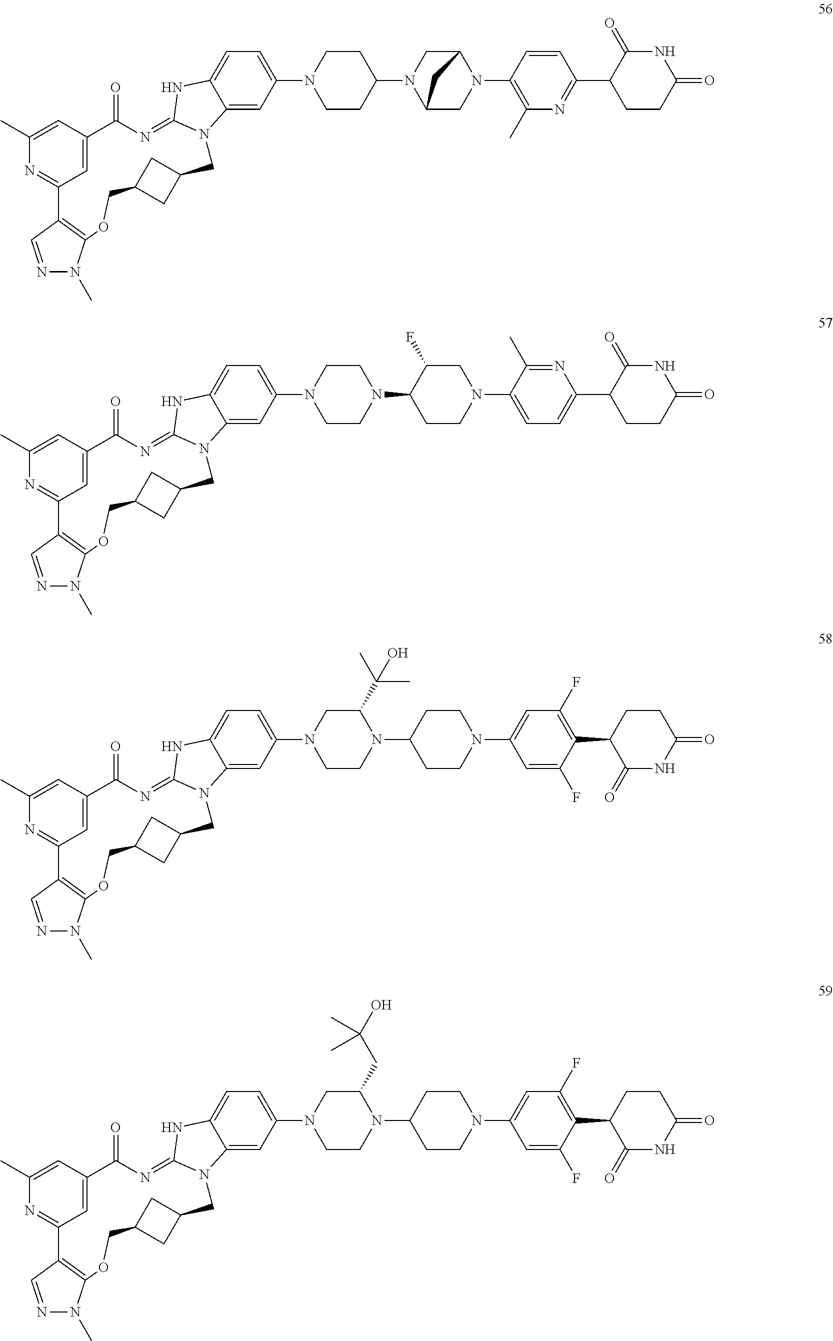

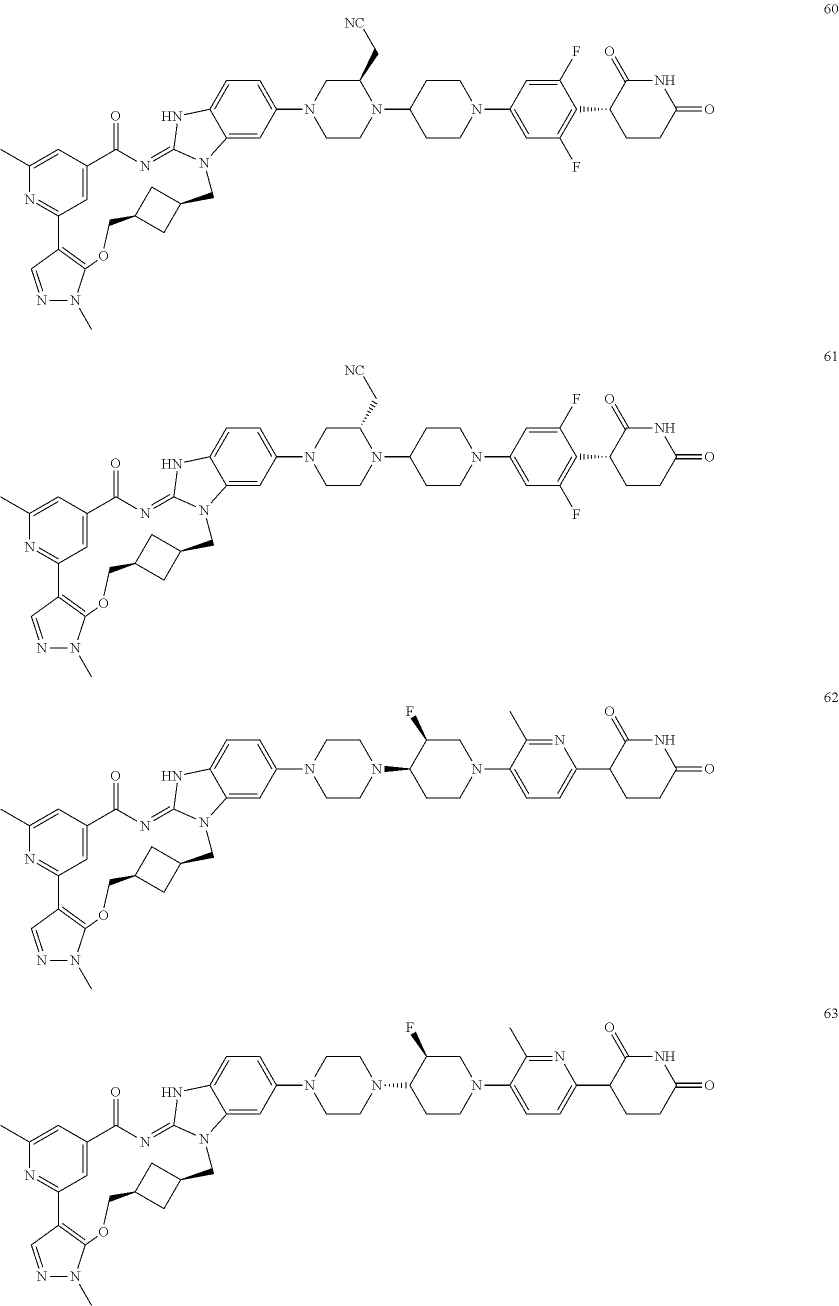

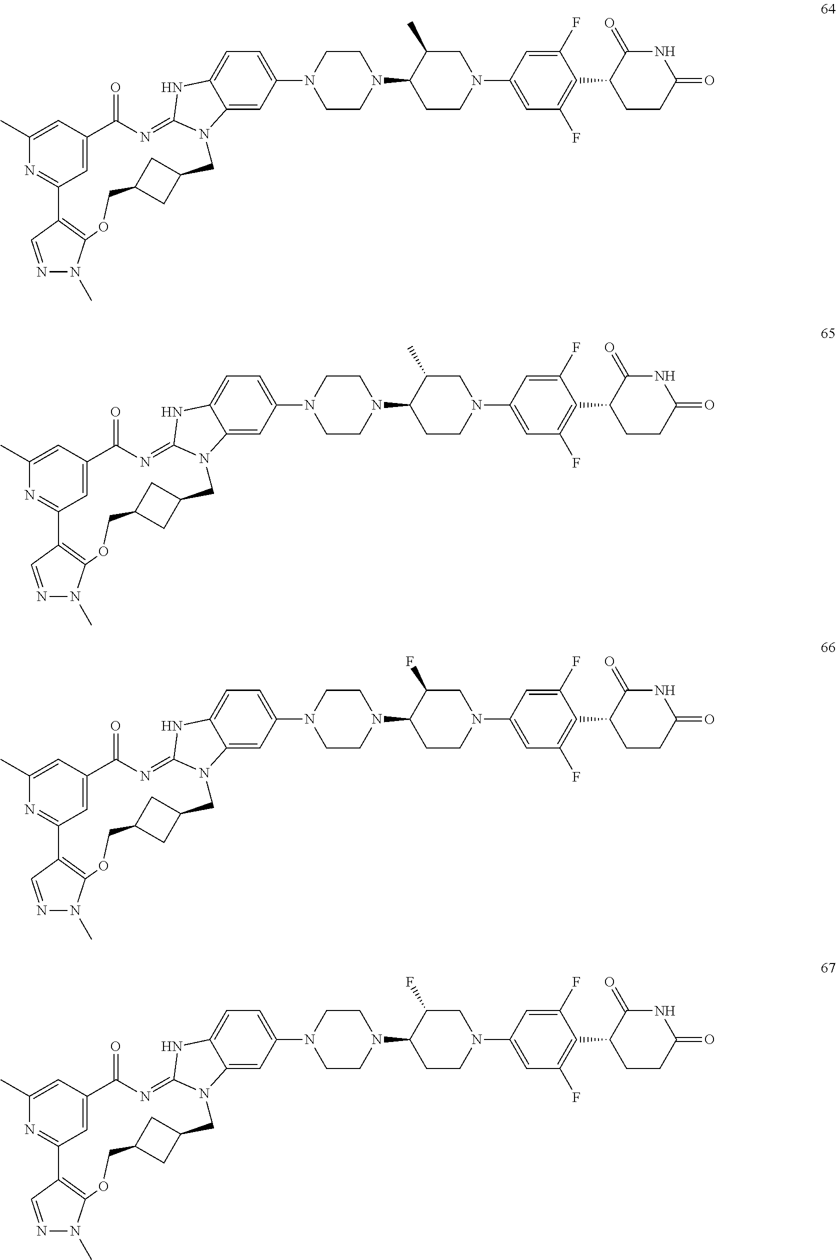

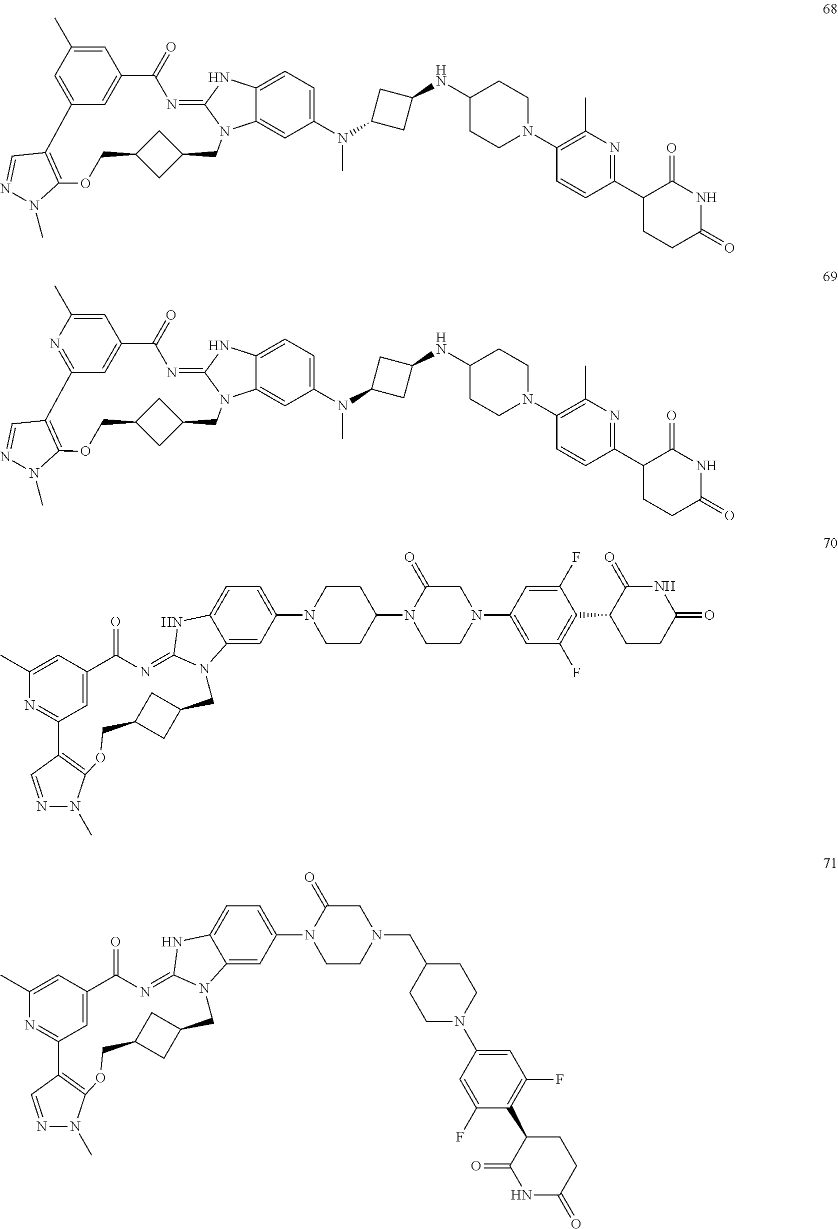

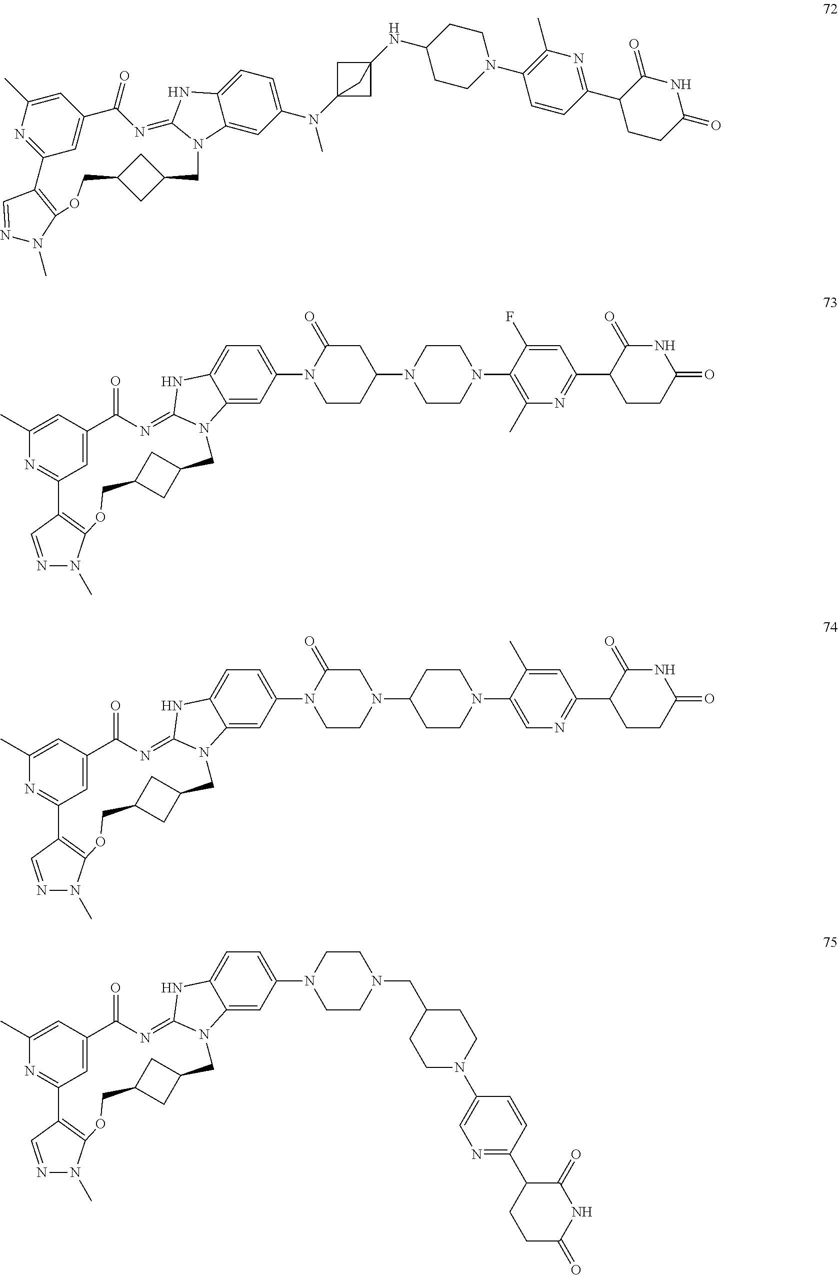 [SMILES: Cc1cc2cc(n1)-c1cnn(C)c1OC[C@H]1C[C@@H](CN3/C(=N/C2=O)Nc2ccc(N(C)C45CC(NC6CCN(c7ccc(C8CCC(=O)NC8=O)nc7C)CC6)(C4)C5)cc23)C1 ; Cc1cc2cc(n1)-c1cnn(C)c1OC[C@H]1C[C@@H](CN3/C(=N/C2=O)Nc2ccc(N4CCC(N5CCN(c6c(F)cc(C7CCC(=O)NC7=O)nc6C)CC5)CC4=O)cc23)C1 ; Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)CC4=O)cc23)C1 ; Cc1cc2cc(n1)-c1cnn(C)c1OC[C@H]1C[C@@H](CN3/C(=N/C2=O)Nc2ccc(N4CCN(CC5CCN(c6ccc(C7CCC(=O)NC7=O)nc6)CC5)CC4)cc23)C1]

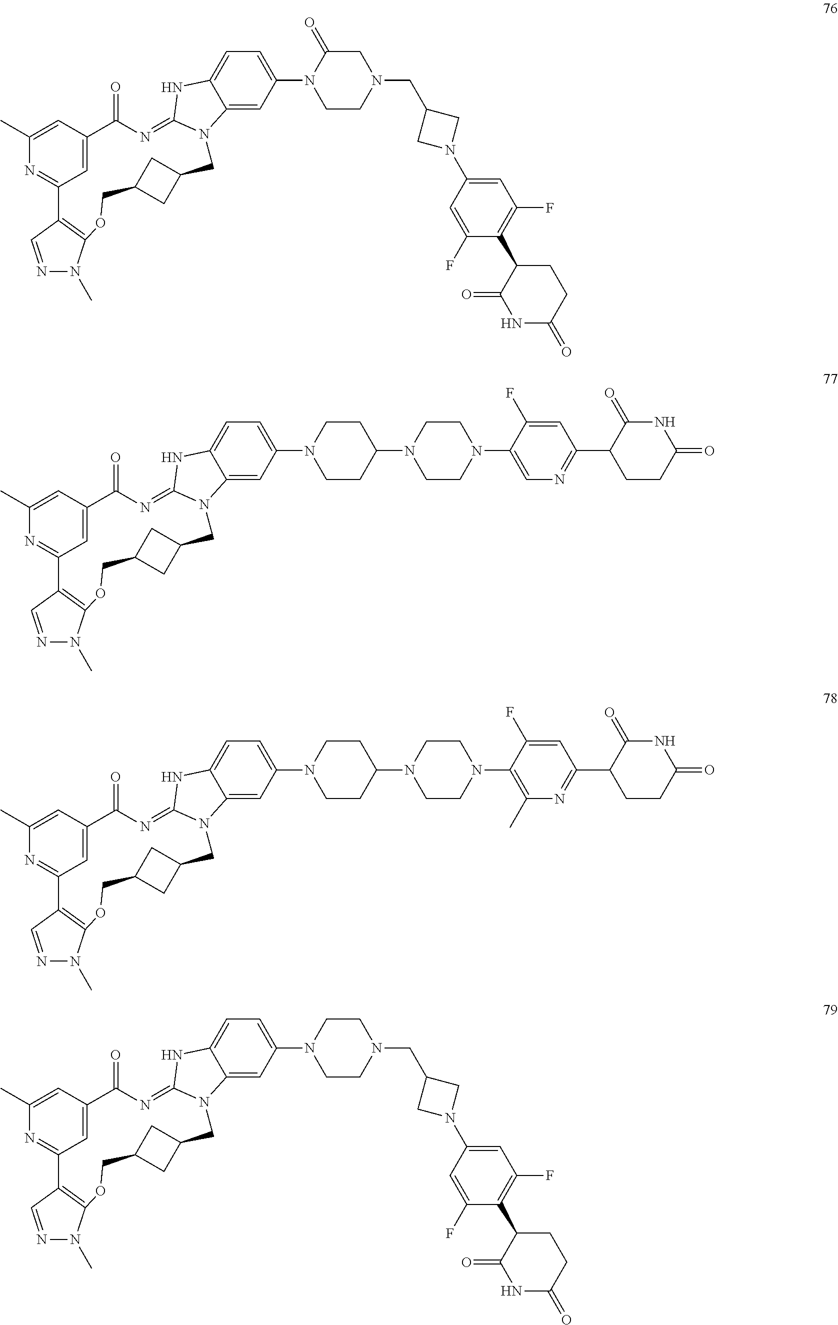 [SMILES: Cc1cc2cc(n1)-c1cnn(C)c1OC[C@H]1C[C@@H](CN3/C(=N/C2=O)Nc2ccc(N4CCN(CC5CN(c6cc(F)c([C@H]7CCC(=O)NC7=O)c(F)c6)C5)CC4=O)cc23)C1 ; Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6F)CC5)CC4)cc23)C1 ; Cc1cc2cc(n1)-c1cnn(C)c1OC[C@H]1C[C@@H](CN3/C(=N/C2=O)Nc2ccc(N4CCC(N5CCN(c6c(F)cc(C7CCC(=O)NC7=O)nc6C)CC5)CC4)cc23)C1 ; Cc1cc2cc(n1)-c1cnn(C)c1OC[C@H]1C[C@@H](CN3/C(=N/C2=O)Nc2ccc(N4CCN(CC5CN(c6cc(F)c([C@H]7CCC(=O)NC7=O)c(F)c6)C5)CC4)cc23)C1]

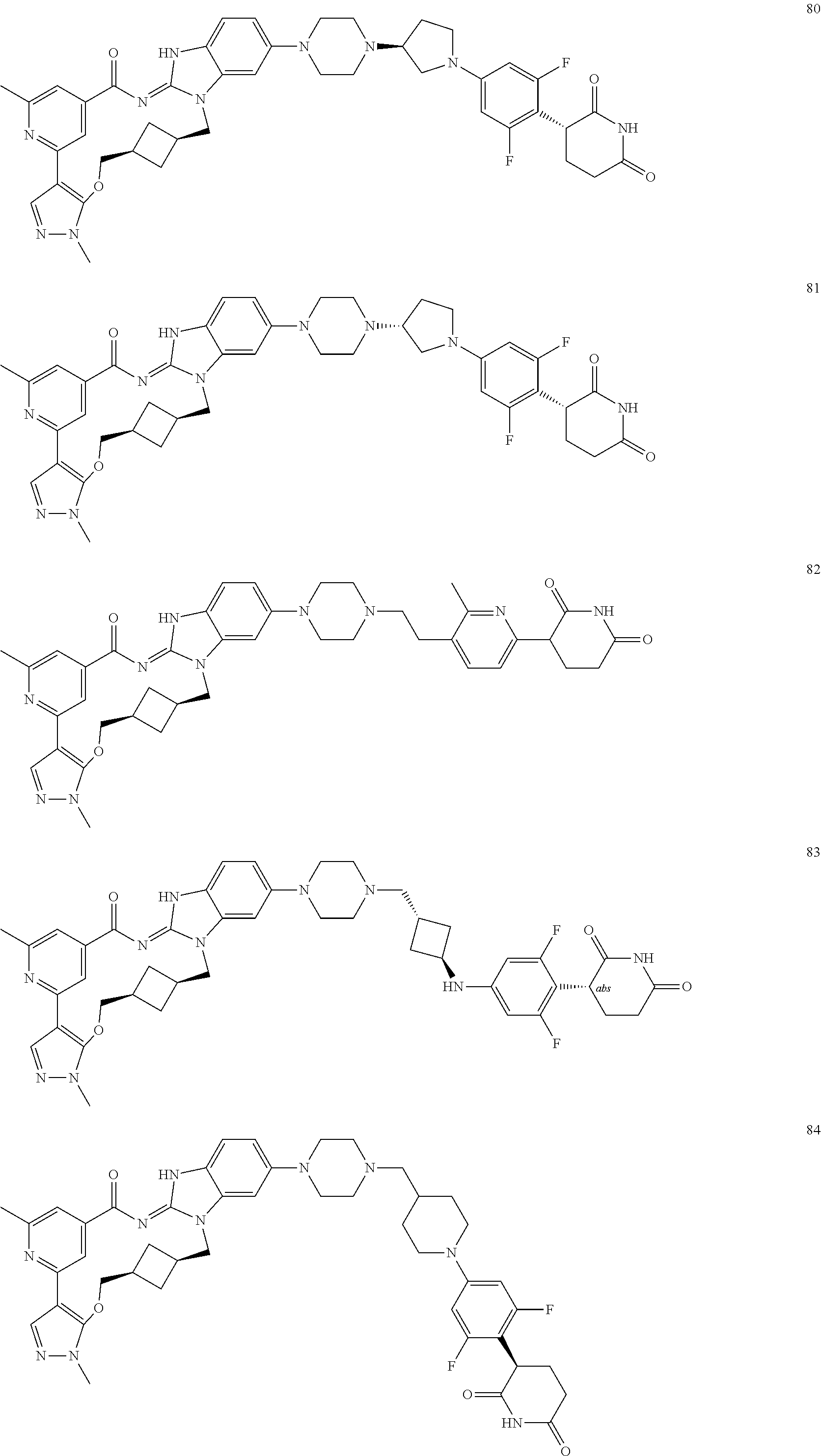

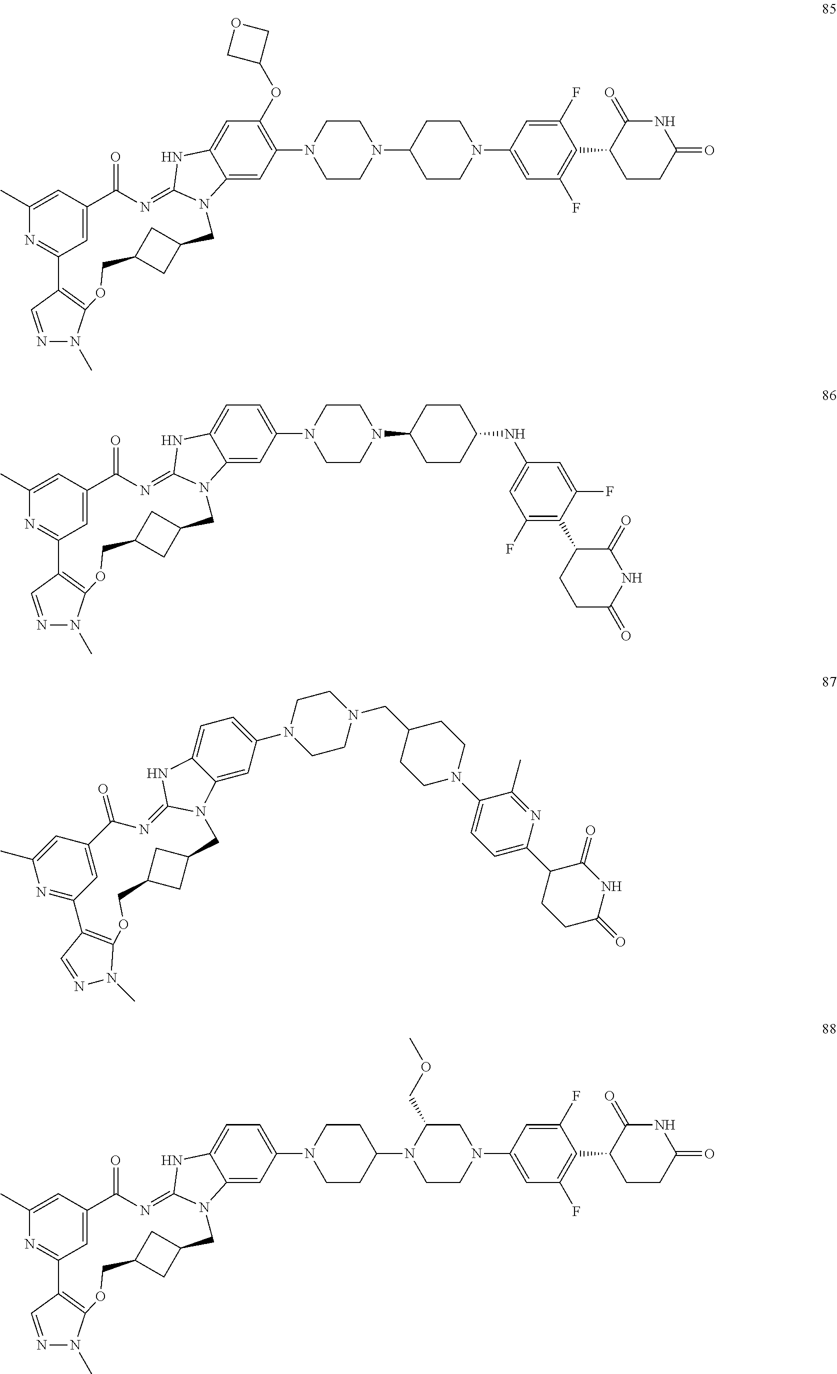

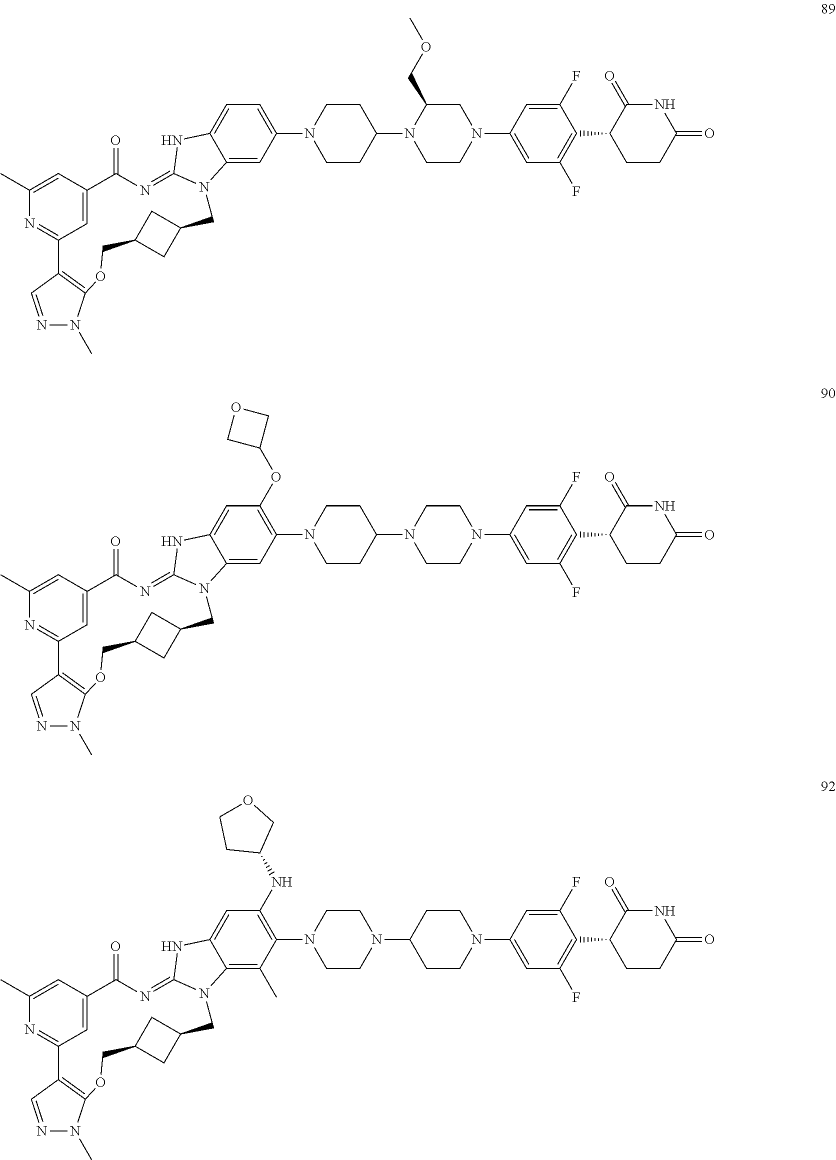 [SMILES: COC[C@H]1CN(c2cc(F)c([C@H]3CCC(=O)NC3=O)c(F)c2)CCN1C1CCN(c2ccc3c(c2)N2C[C@H]4C[C@@H](COc5c(cnn5C)-c5cc(cc(C)n5)C(=O)/N=C/2N3)C4)CC1 ; Cc1cc2cc(n1)-c1cnn(C)c1OC[C@H]1C[C@@H](CN3/C(=N/C2=O)Nc2cc(OC4COC4)c(N4CCC(N5CCN(c6cc(F)c([C@H]7CCC(=O)NC7=O)c(F)c6)CC5)CC4)cc23)C1 ; Cc1cc2cc(n1)-c1cnn(C)c1OC[C@H]1C[C@@H](CN3/C(=N/C2=O)Nc2cc(N[C@@H]4CCOC4)c(N4CCN(C5CCN(c6cc(F)c([C@H]7CCC(=O)NC7=O)c(F)c6)CC5)CC4)c(C)c23)C1]

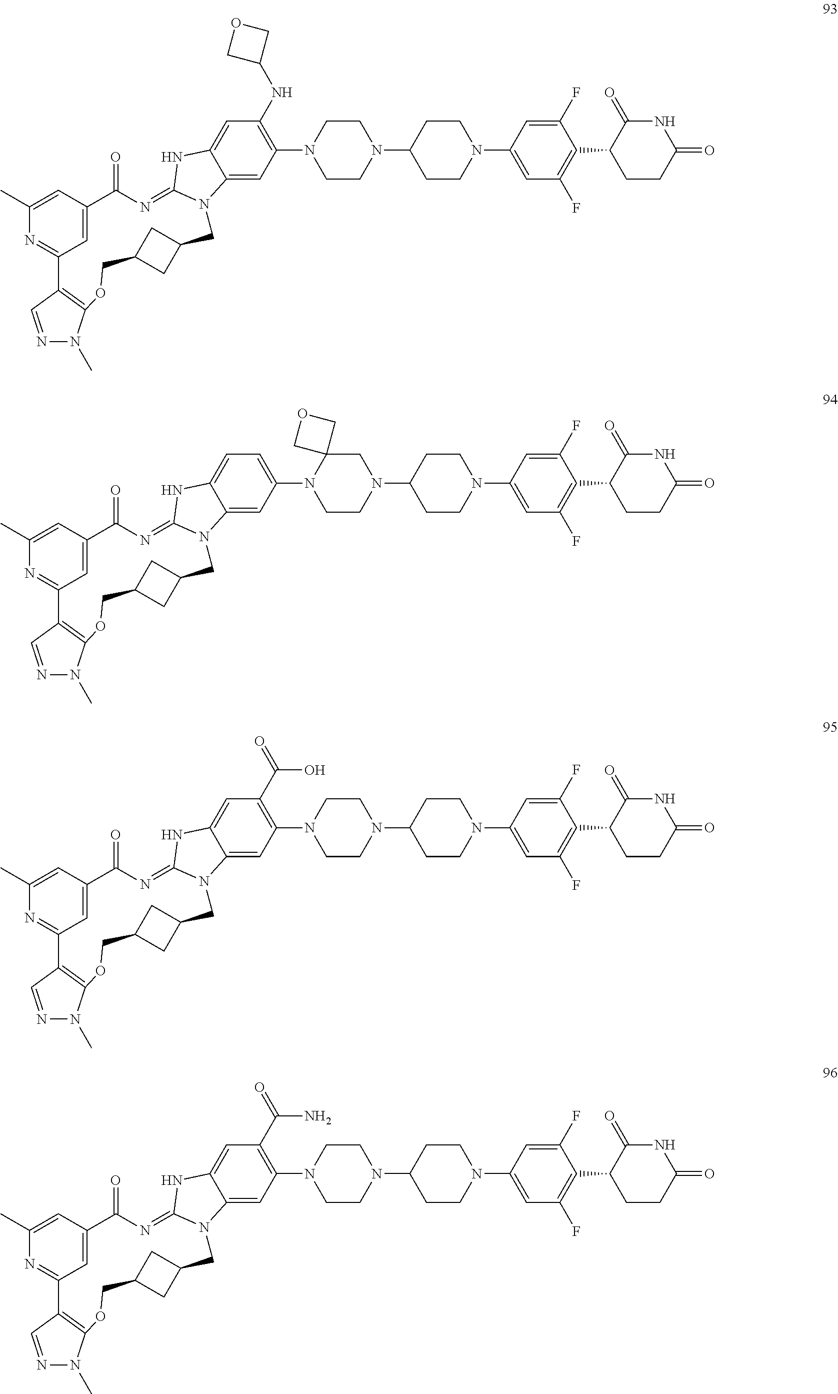

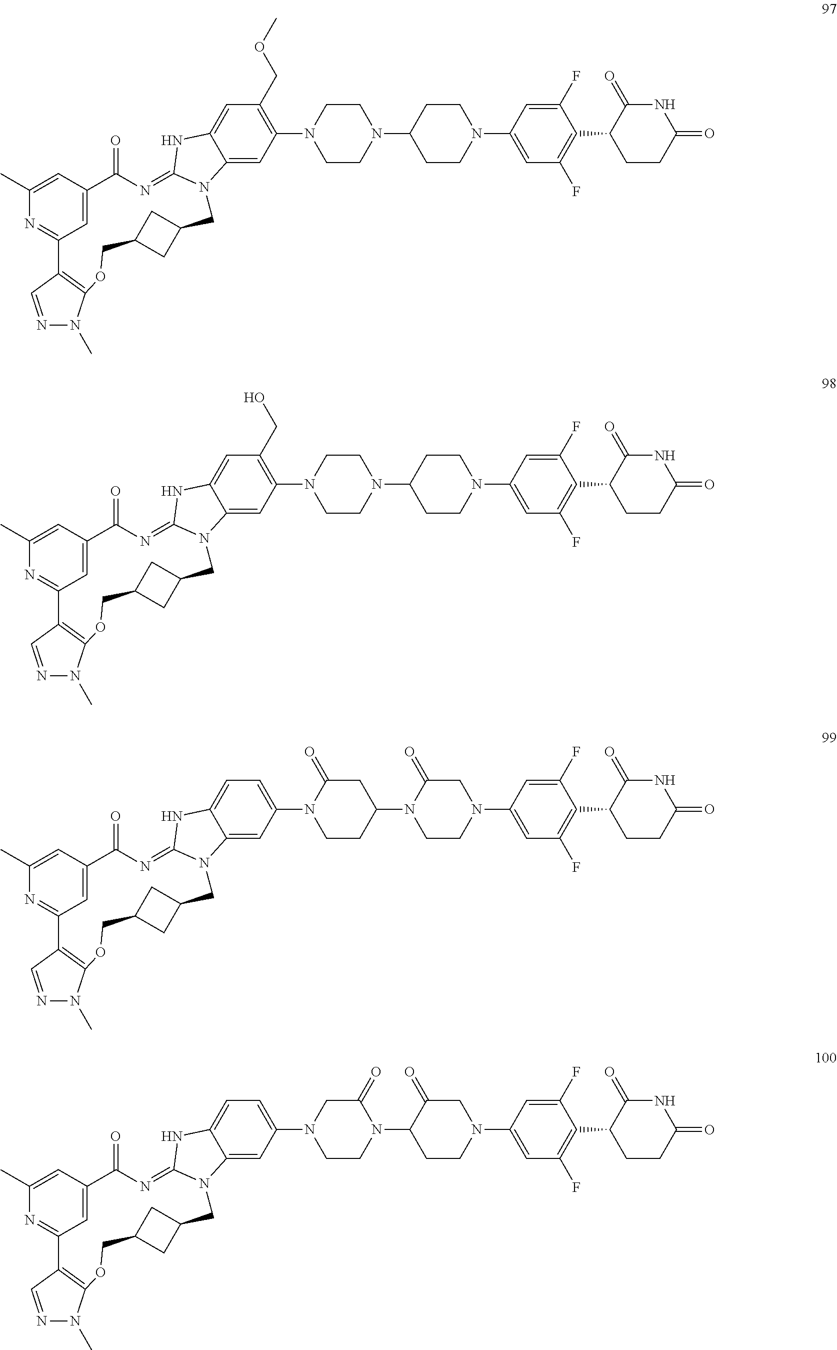

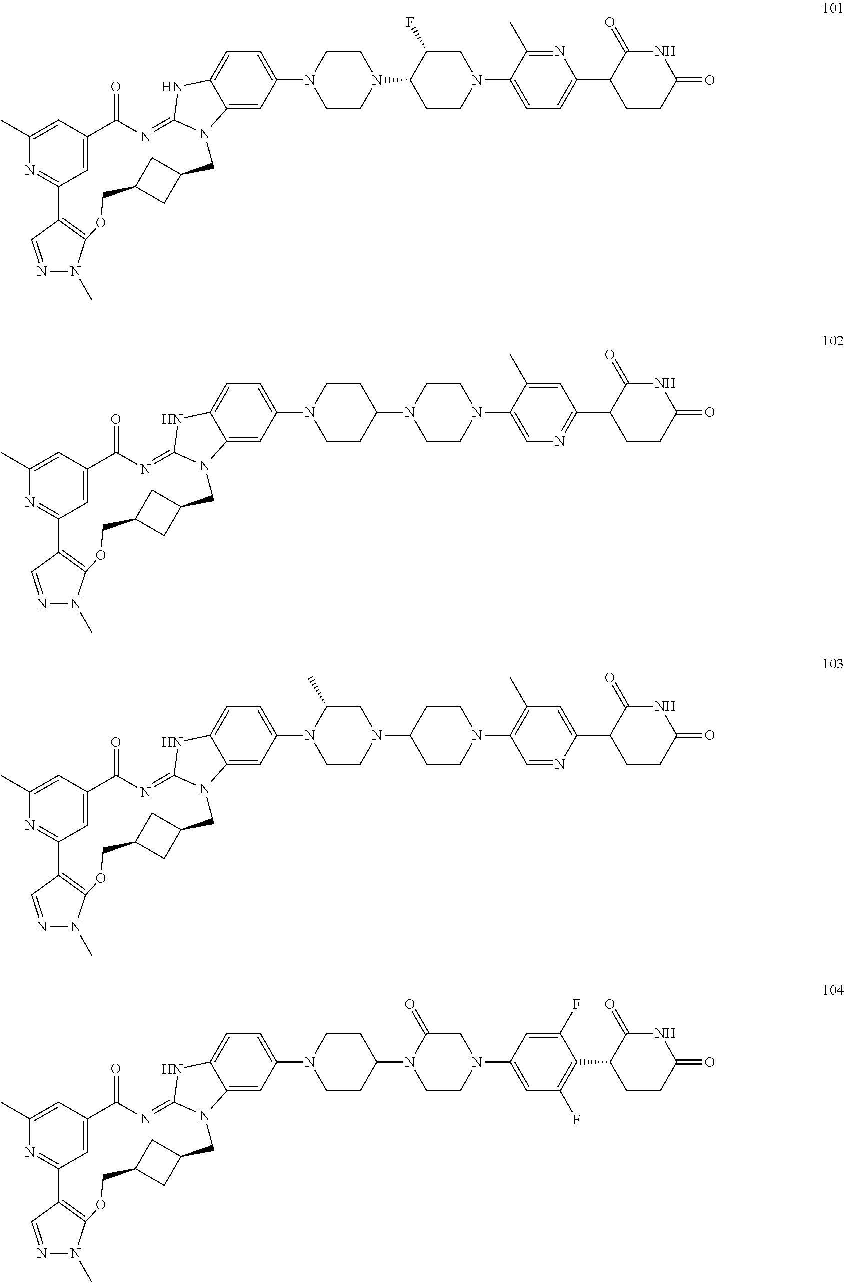 [SMILES: Cc1cc2cc(n1)-c1cnn(C)c1OC[C@H]1C[C@@H](CN3/C(=N/C2=O)Nc2ccc(N4CCN([C@H]5CCN(c6ccc(C7CCC(=O)NC7=O)nc6C)C[C@H]5F)CC4)cc23)C1 ; Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6C)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)C[C@H]4C)cc23)C1 ; Cc1cc2cc(n1)-c1cnn(C)c1OC[C@H]1C[C@@H](CN3/C(=N/C2=O)Nc2ccc(N4CCC(N5CCN(c6cc(F)c([C@H]7CCC(=O)NC7=O)c(F)c6)CC5=O)CC4)cc23)C1]

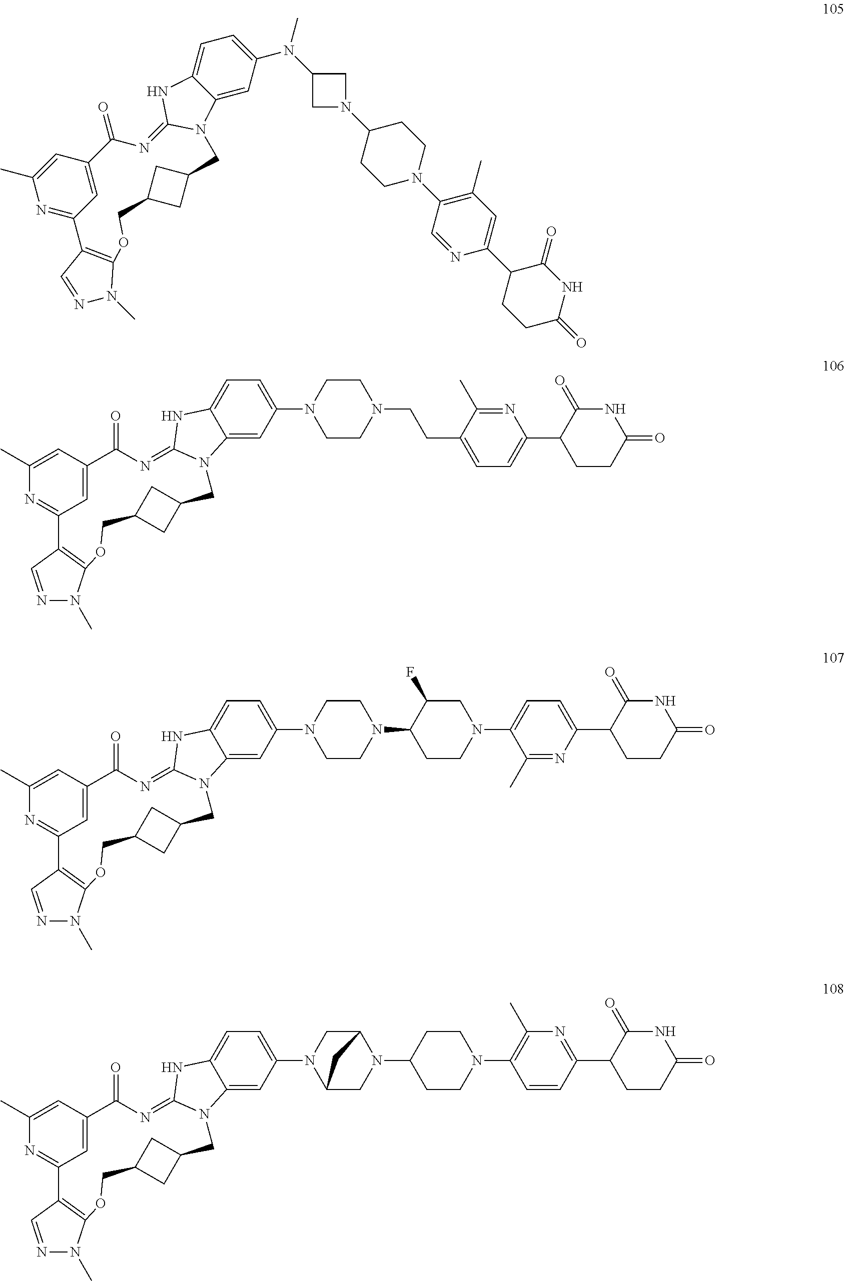

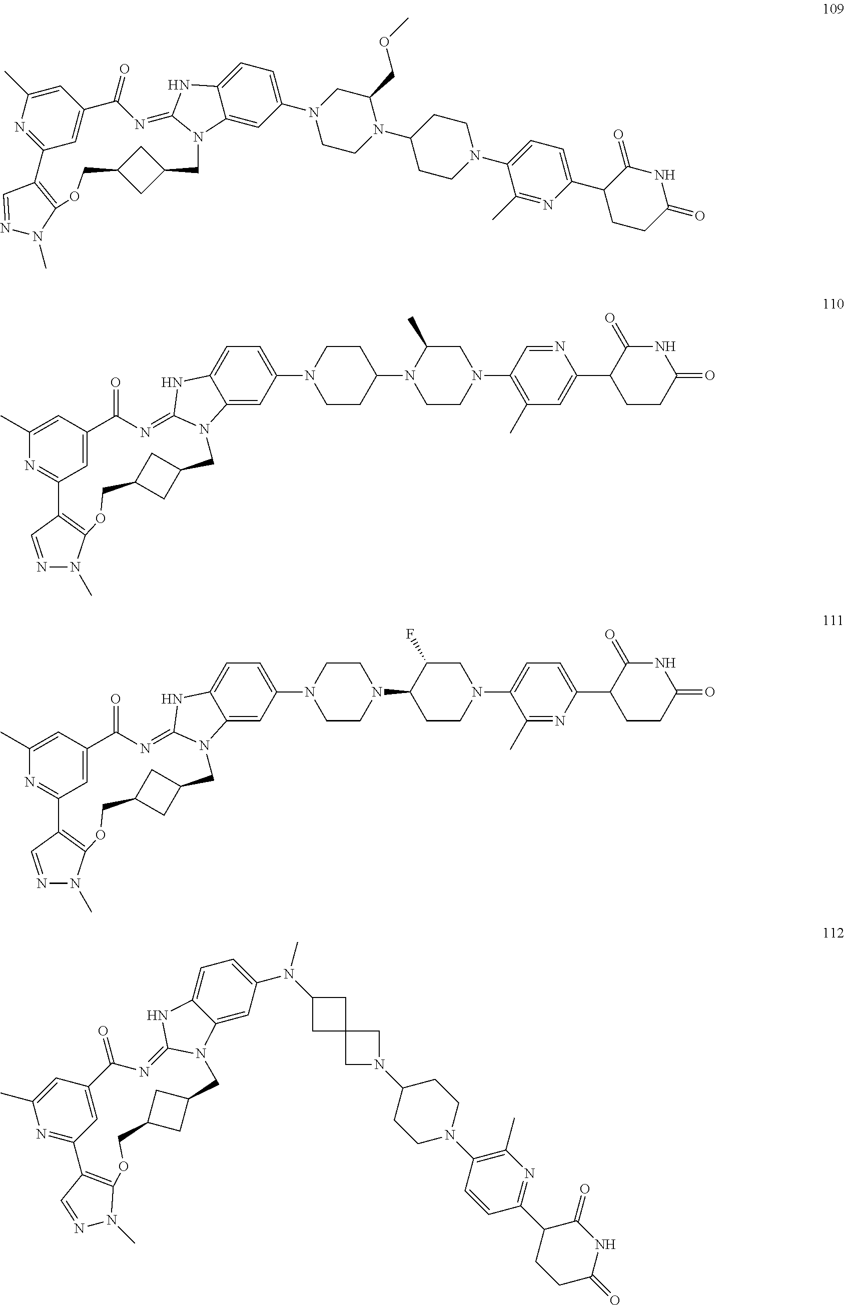 [SMILES: COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2ccc(C3CCC(=O)NC3=O)nc2C)CC1 ; Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6C)C[C@@H]5C)CC4)cc23)C1 ; Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)C[C@H]5F)CC4)cc23)C1 ; Cc1cc2cc(n1)-c1cnn(C)c1OC[C@H]1C[C@@H](CN3/C(=N/C2=O)Nc2ccc(N(C)C4CC5(C4)CN(C4CCN(c6ccc(C7CCC(=O)NC7=O)nc6C)CC4)C5)cc23)C1]

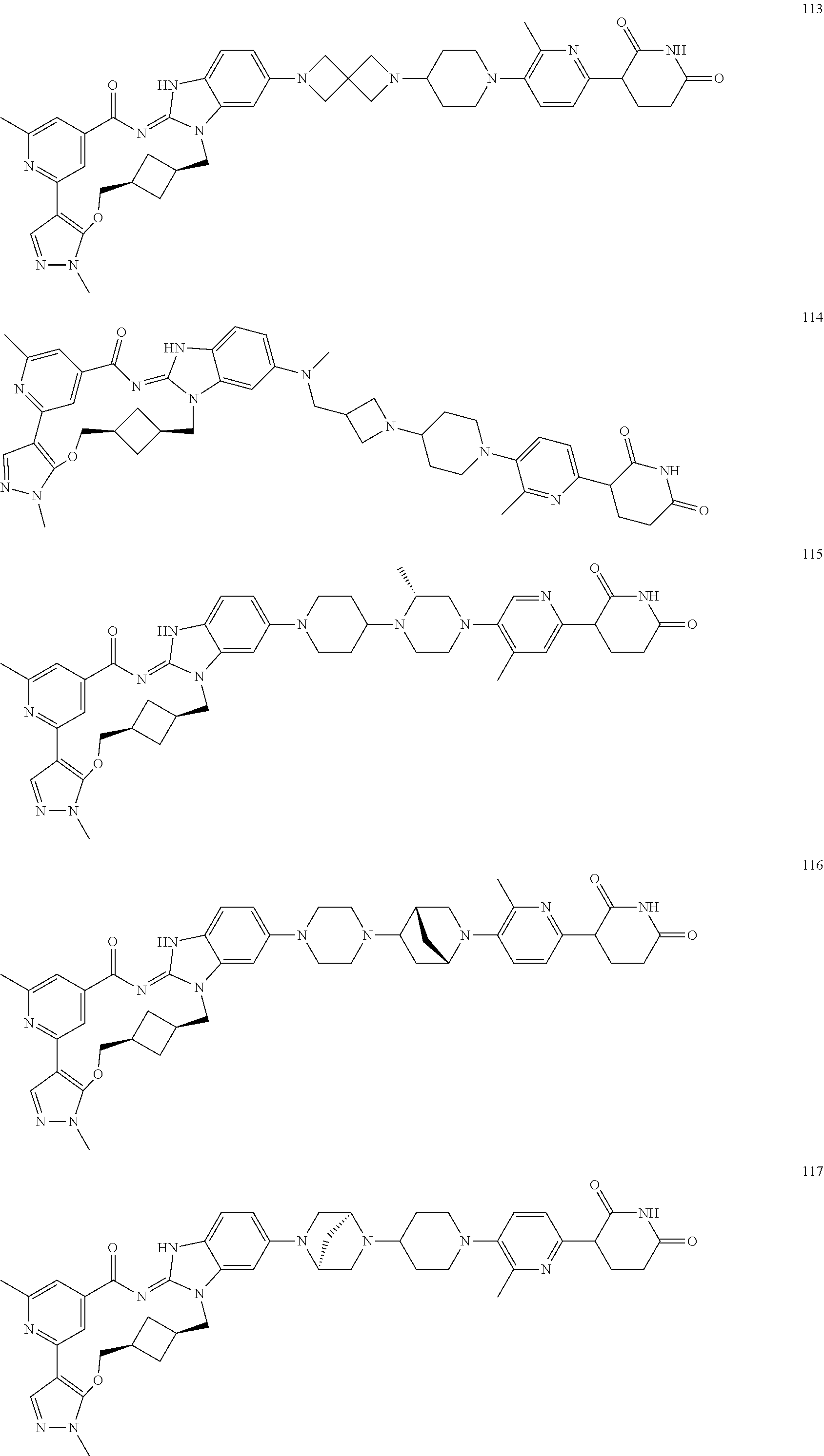

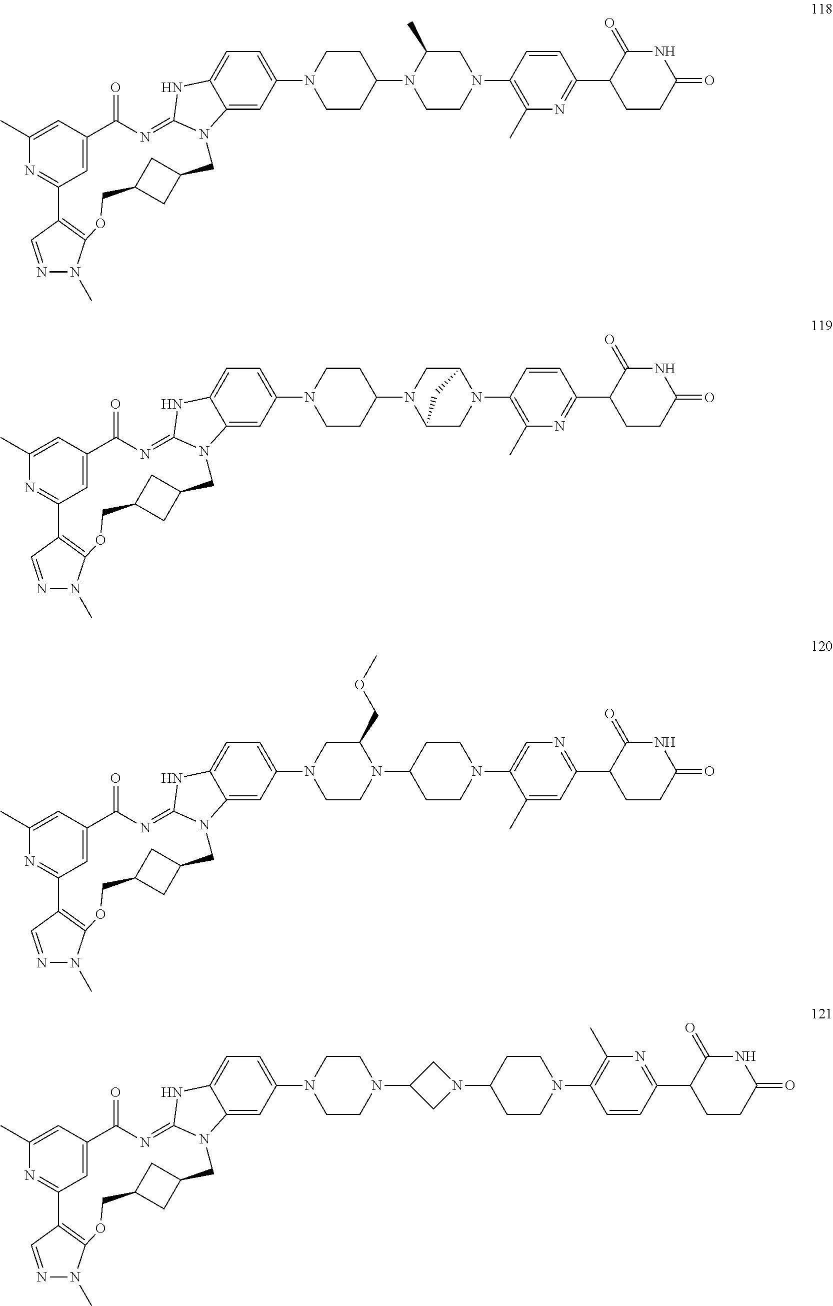 [SMILES: Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)C[C@@H]5C)CC4)cc23)C1 ; Cc1cc2cc(n1)-c1cnn(C)c1OC[C@H]1C[C@@H](CN3/C(=N/C2=O)Nc2ccc(N4CCC(N5C[C@@H]6C[C@H]5CN6c5ccc(C6CCC(=O)NC6=O)nc5C)CC4)cc23)C1 ; COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2cnc(C3CCC(=O)NC3=O)cc2C)CC1 ; Cc1cc2cc(n1)-c1cnn(C)c1OC[C@H]1C[C@@H](CN3/C(=N/C2=O)Nc2ccc(N4CCN(C5CN(C6CCN(c7ccc(C8CCC(=O)NC8=O)nc7C)CC6)C5)CC4)cc23)C1]

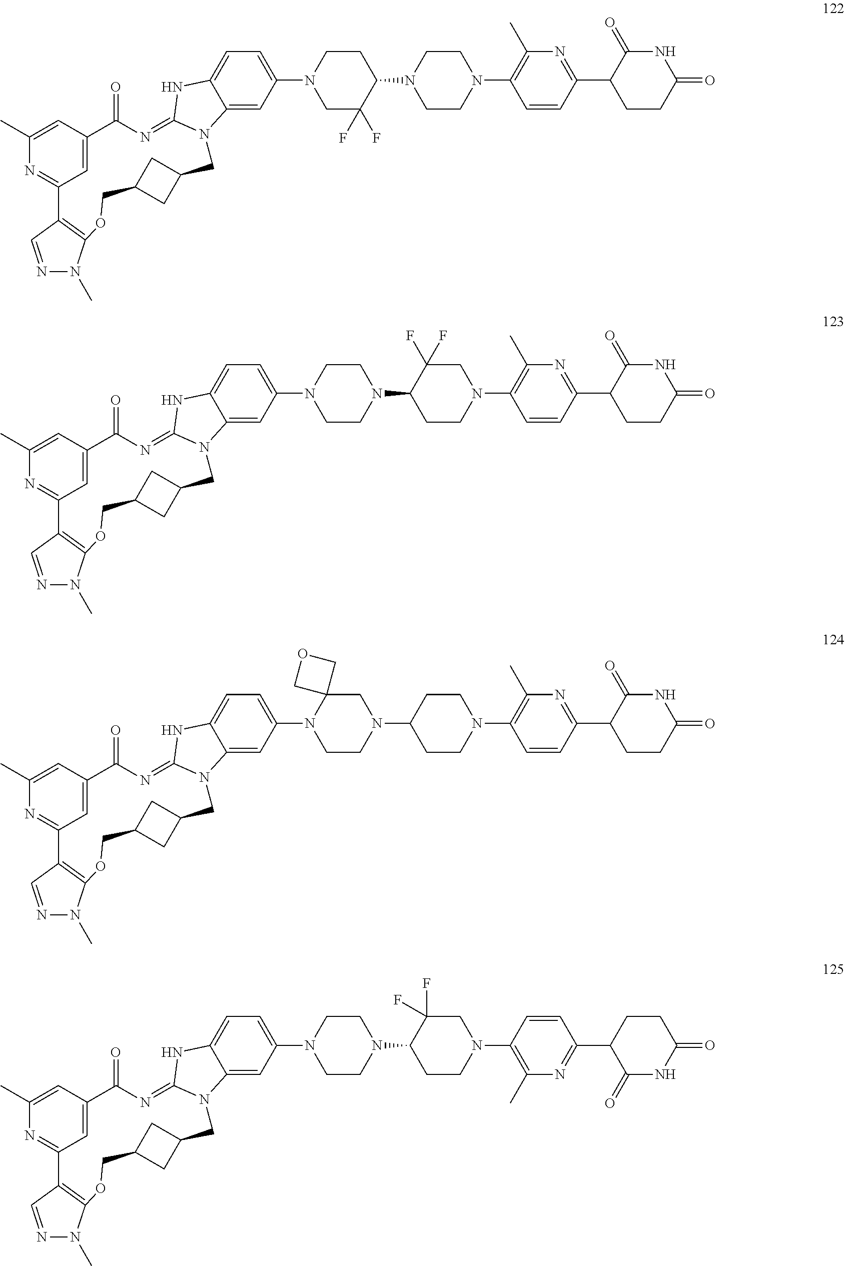 [SMILES: Cc1cc2cc(n1)-c1cnn(C)c1OC[C@H]1C[C@@H](CN3/C(=N/C2=O)Nc2ccc(N4CC[C@H](N5CCN(c6ccc(C7CCC(=O)NC7=O)nc6C)CC5)C(F)(F)C4)cc23)C1 ; Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)CC5(F)F)CC4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC45COC5)cc23)C1 ; Cc1cc2cc(n1)-c1cnn(C)c1OC[C@H]1C[C@@H](CN3/C(=N/C2=O)Nc2ccc(N4CCN([C@H]5CCN(c6ccc(C7CCC(=O)NC7=O)nc6C)CC5(F)F)CC4)cc23)C1]

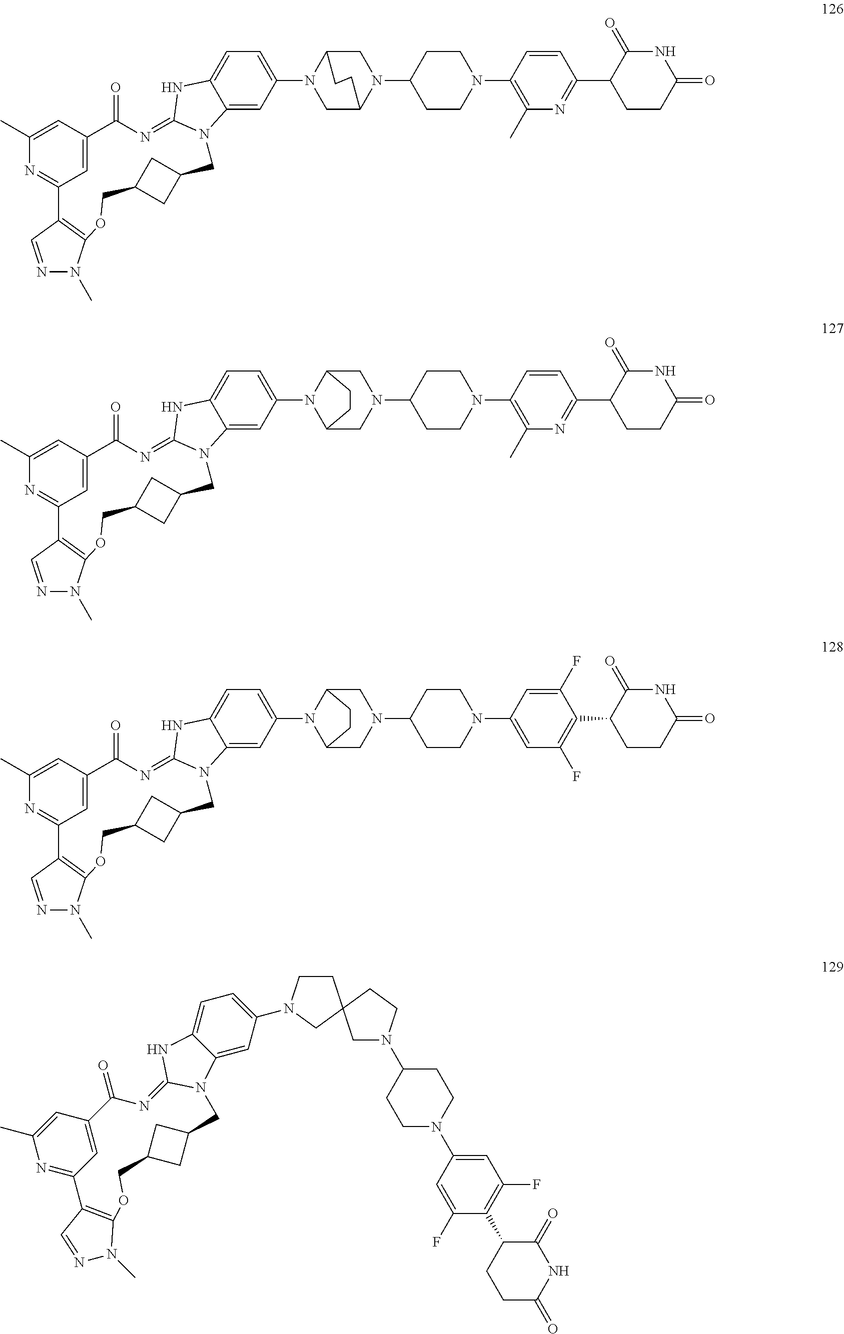 [SMILES: Cc1cc2cc(n1)-c1cnn(C)c1OC[C@H]1C[C@@H](CN3/C(=N/C2=O)Nc2ccc(N4CC5CCC4CN5C4CCN(c5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4C5CCC4CN(C4CCN(c6ccc(C7CCC(=O)NC7=O)nc6C)CC4)C5)cc23)C1 ; Cc1cc2cc(n1)-c1cnn(C)c1OC[C@H]1C[C@@H](CN3/C(=N/C2=O)Nc2ccc(N4C5CCC4CN(C4CCN(c6cc(F)c([C@H]7CCC(=O)NC7=O)c(F)c6)CC4)C5)cc23)C1 ; Cc1cc2cc(n1)-c1cnn(C)c1OC[C@H]1C[C@@H](CN3/C(=N/C2=O)Nc2ccc(N4CCC5(CCN(C6CCN(c7cc(F)c([C@H]8CCC(=O)NC8=O)c(F)c7)CC6)C5)C4)cc23)C1]

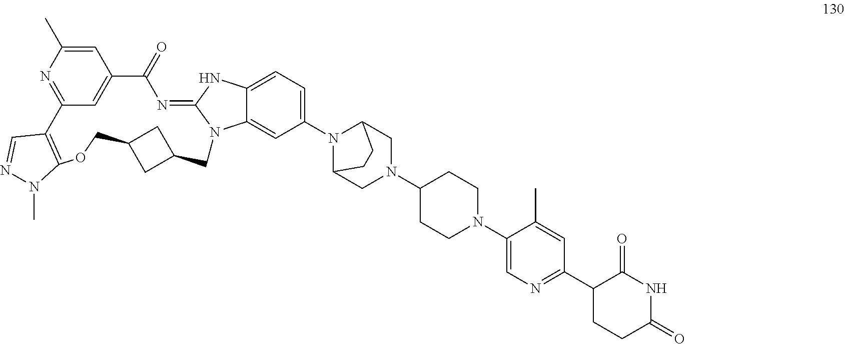 [SMILES: Cc1cc2cc(n1)-c1cnn(C)c1OC[C@H]1C[C@@H](CN3/C(=N/C2=O)Nc2ccc(N4C5CCC4CN(C4CCN(c6cnc(C7CCC(=O)NC7=O)cc6C)CC4)C5)cc23)C1]

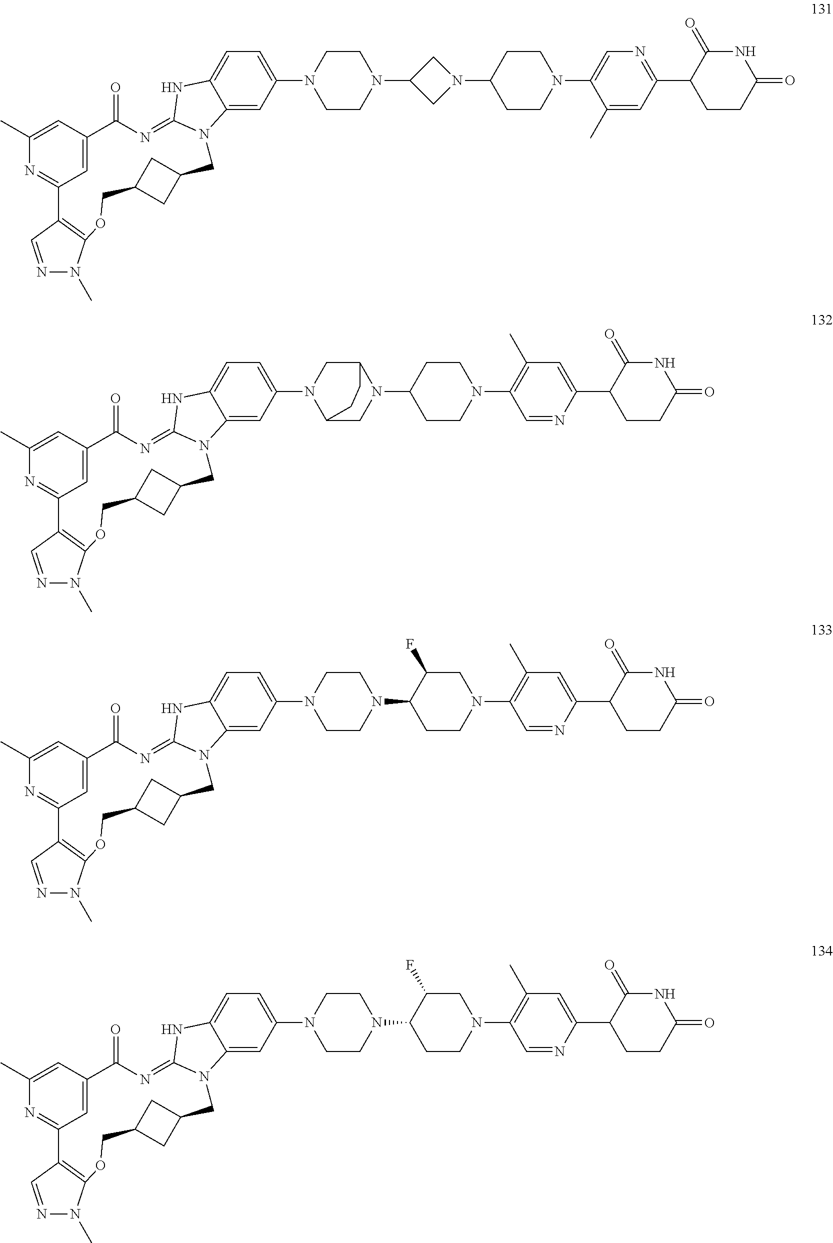 [SMILES: Cc1cc2cc(n1)-c1cnn(C)c1OC[C@H]1C[C@@H](CN3/C(=N/C2=O)Nc2ccc(N4CCN(C5CN(C6CCN(c7cnc(C8CCC(=O)NC8=O)cc7C)CC6)C5)CC4)cc23)C1 ; Cc1cc2cc(n1)-c1cnn(C)c1OC[C@H]1C[C@@H](CN3/C(=N/C2=O)Nc2ccc(N4CC5CCC4CN5C4CCN(c5cnc(C6CCC(=O)NC6=O)cc5C)CC4)cc23)C1 ; Cc1cc2cc(n1)-c1cnn(C)c1OC[C@H]1C[C@@H](CN3/C(=N/C2=O)Nc2ccc(N4CCN([C@@H]5CCN(c6cnc(C7CCC(=O)NC7=O)cc6C)C[C@@H]5F)CC4)cc23)C1 ; Cc1cc2cc(n1)-c1cnn(C)c1OC[C@H]1C[C@@H](CN3/C(=N/C2=O)Nc2ccc(N4CCN([C@H]5CCN(c6cnc(C7CCC(=O)NC7=O)cc6C)C[C@H]5F)CC4)cc23)C1]

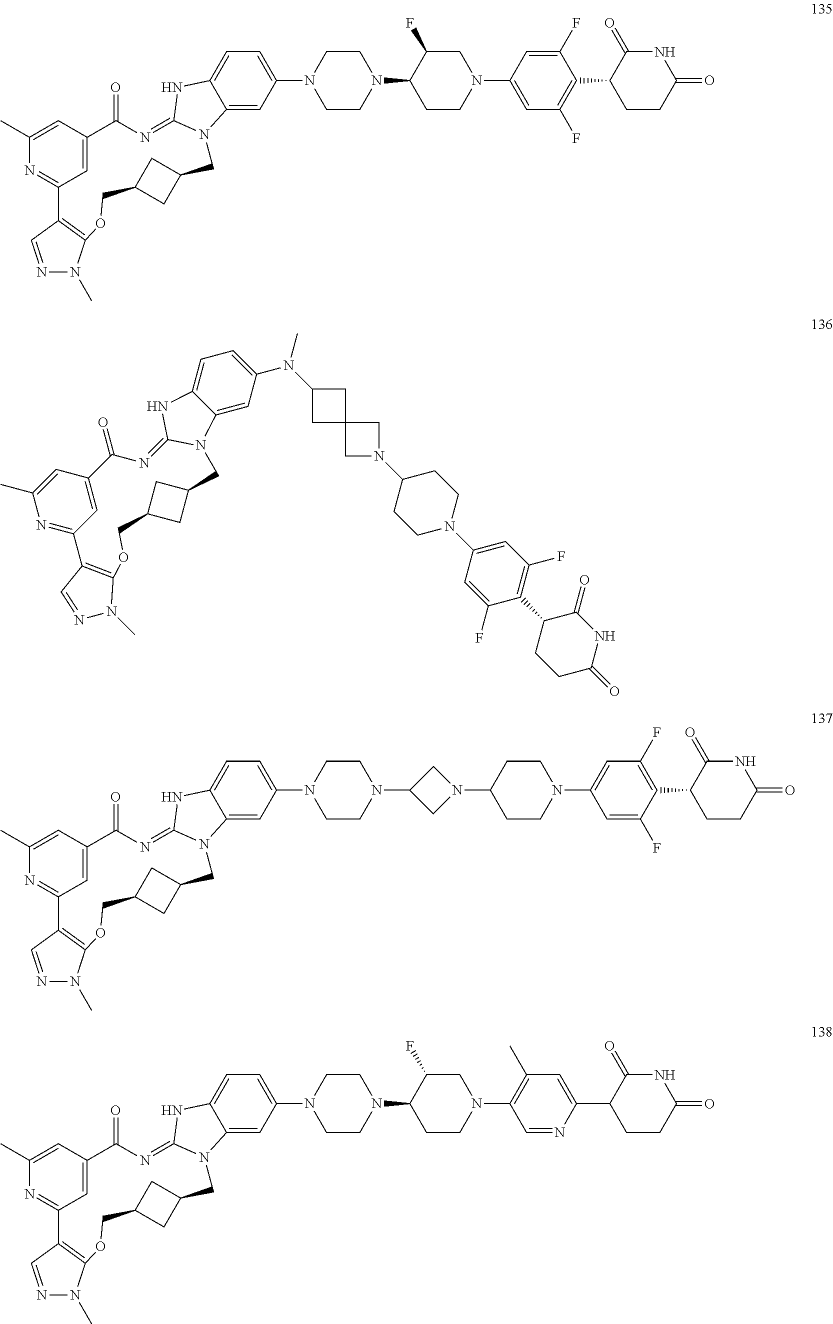

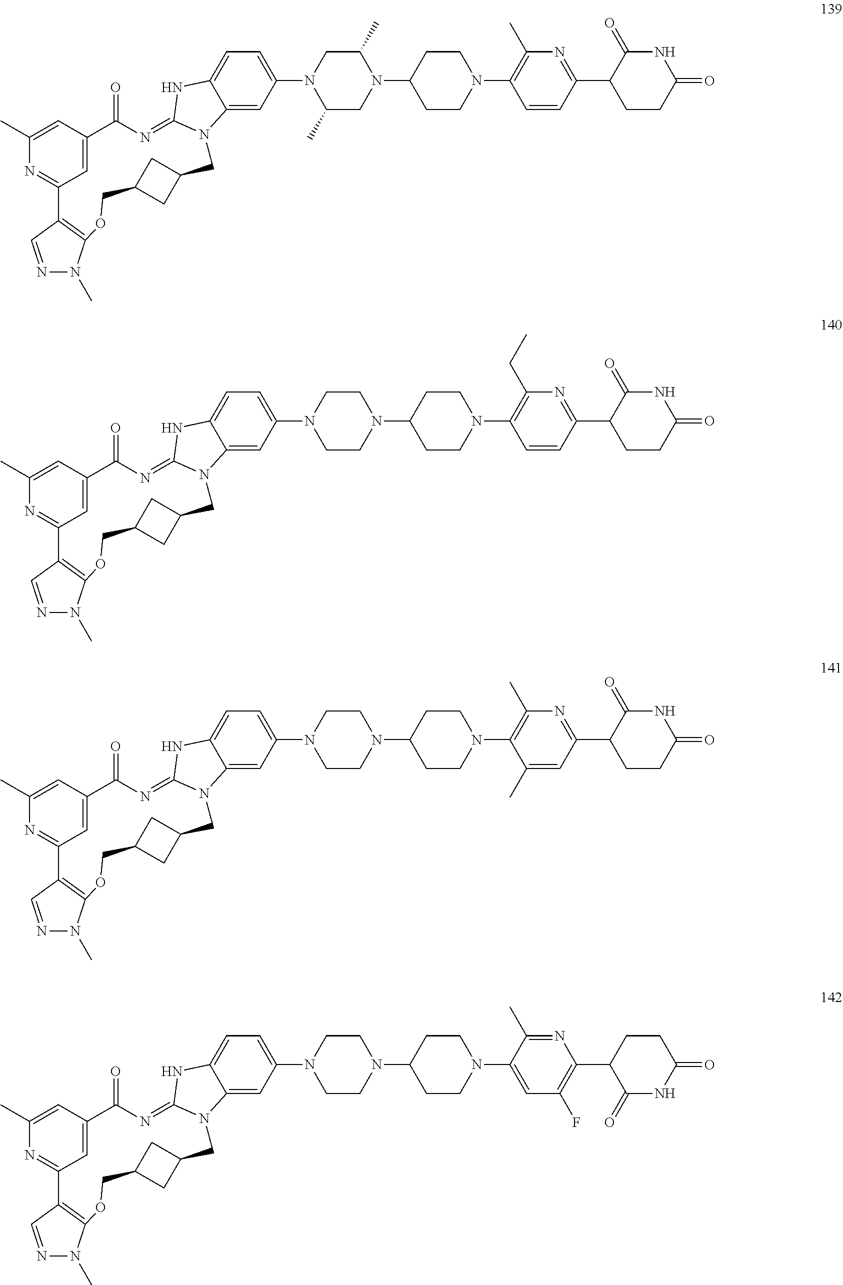 [SMILES: Cc1cc2cc(n1)-c1cnn(C)c1OC[C@H]1C[C@@H](CN3/C(=N/C2=O)Nc2ccc(N4C[C@H](C)N(C5CCN(c6ccc(C7CCC(=O)NC7=O)nc6C)CC5)C[C@@H]4C)cc23)C1 ; CCc1nc(C2CCC(=O)NC2=O)ccc1N1CCC(N2CCN(c3ccc4c(c3)N3C[C@H]5C[C@@H](COc6c(cnn6C)-c6cc(cc(C)n6)C(=O)/N=C/3N4)C5)CC2)CC1 ; Cc1cc2cc(n1)-c1cnn(C)c1OC[C@H]1C[C@@H](CN3/C(=N/C2=O)Nc2ccc(N4CCN(C5CCN(c6c(C)cc(C7CCC(=O)NC7=O)nc6C)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6cc(F)c(C7CCC(=O)NC7=O)nc6C)CC5)CC4)cc23)C1]

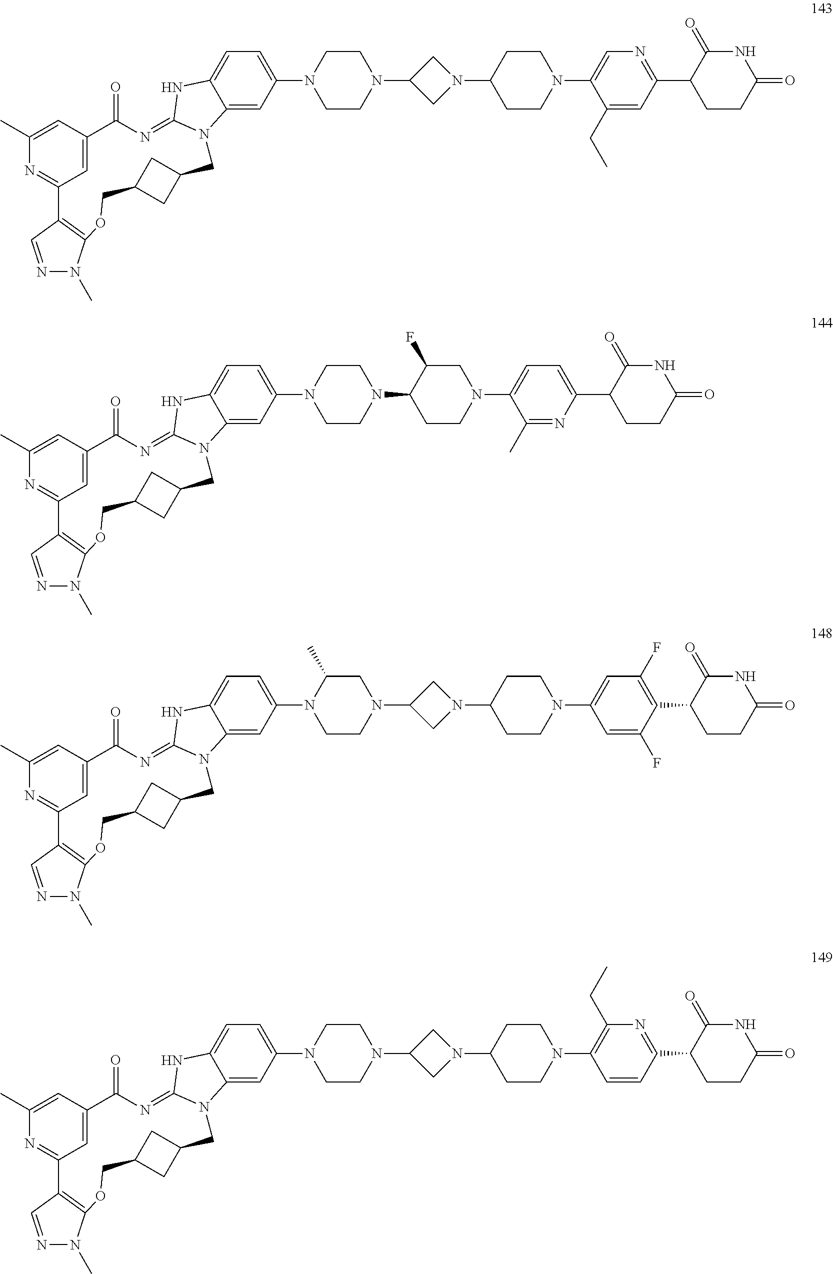

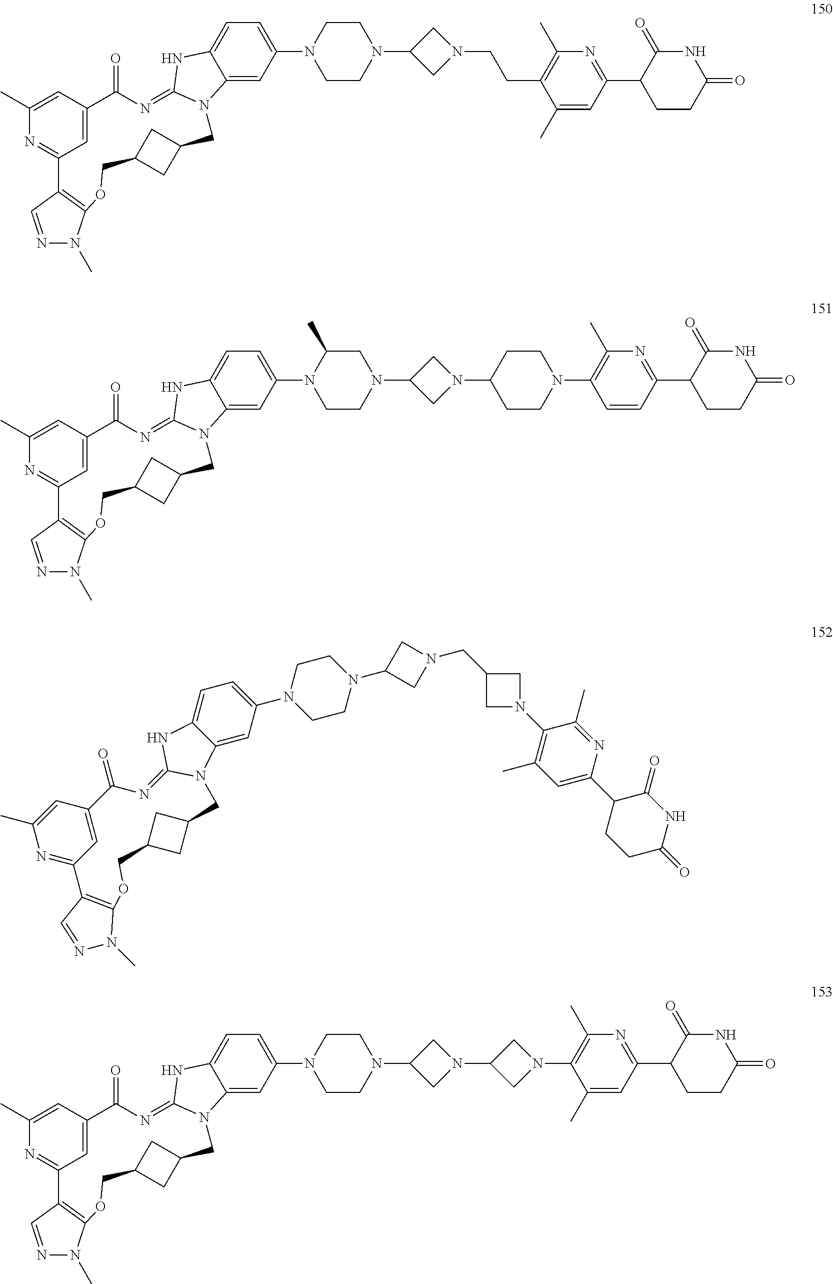 [SMILES: Cc1cc2cc(n1)-c1cnn(C)c1OC[C@H]1C[C@@H](CN3/C(=N/C2=O)Nc2ccc(N4CCN(C5CN(CCc6c(C)cc(C7CCC(=O)NC7=O)nc6C)C5)CC4)cc23)C1 ; Cc1cc2cc(n1)-c1cnn(C)c1OC[C@H]1C[C@@H](CN3/C(=N/C2=O)Nc2ccc(N4CCN(C5CN(C6CCN(c7ccc(C8CCC(=O)NC8=O)nc7C)CC6)C5)C[C@@H]4C)cc23)C1 ; Cc1cc2cc(n1)-c1cnn(C)c1OC[C@H]1C[C@@H](CN3/C(=N/C2=O)Nc2ccc(N4CCN(C5CN(CC6CN(c7c(C)cc(C8CCC(=O)NC8=O)nc7C)C6)C5)CC4)cc23)C1 ; Cc1cc2cc(n1)-c1cnn(C)c1OC[C@H]1C[C@@H](CN3/C(=N/C2=O)Nc2ccc(N4CCN(C5CN(C6CN(c7c(C)cc(C8CCC(=O)NC8=O)nc7C)C6)C5)CC4)cc23)C1]

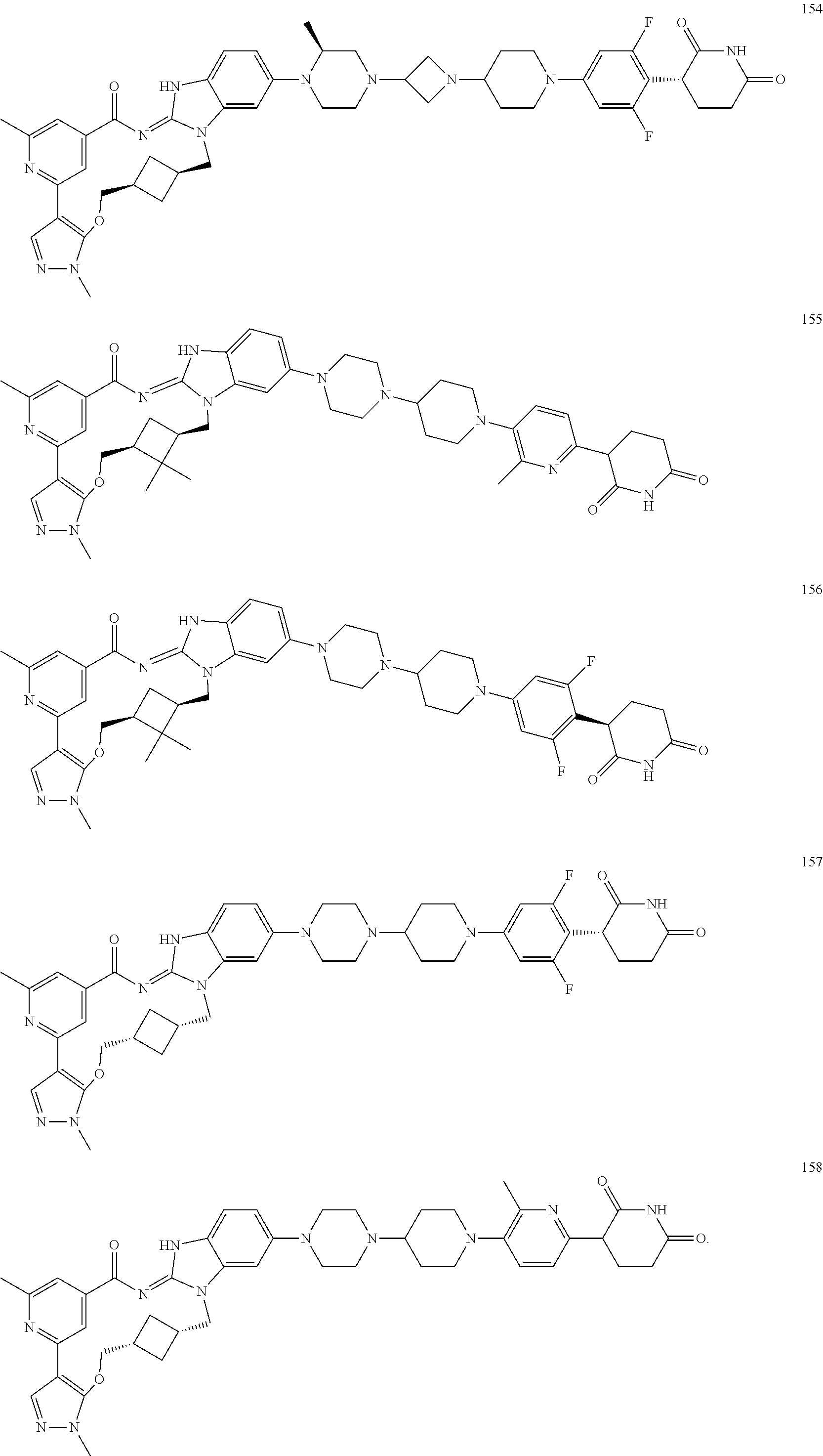

Aspect 36. A pharmaceutical composition comprising a compound of any one of Aspects 1-32 or a pharmaceutically acceptable salt, stereoisomer, tautomer or prodrug thereof, together with a pharmaceutically acceptable excipient.

Aspect 37. A method of treating a disease that can be affected by EGFR modulation, comprises administrating a subject in need thereof an effective amount of a compound of any one of Aspects 1-32 or a pharmaceutically acceptable salt, stereoisomer, tautomer or prodrug thereof.

Aspect 38. The method of Aspect 34, wherein the disease is selected from cancer, preferred pancreatic cancer, breast cancer, glioblastoma multiforme, head and neck cancer, or non-small cell lung cancer.

Aspect 39. Use of a compound of any one of Aspects 1-32 or a pharmaceutically acceptable salt, stereoisomer, tautomer or prodrug thereof in the preparation of a medicament for treating a disease that can be affected by EGFR modulation.

Aspect 40. The use of Aspect 36, wherein the disease is cancer, preferred pancreatic cancer, breast cancer, glioblastoma multiforme, head and neck cancer, or non-small cell lung cancer.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkynylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from: 5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:

5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;

7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and

11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “H” or “hydrogen” disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H, et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W, Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable. For another example, compounds including pyrazolyl may under go tautomerism to form a different ring like below:

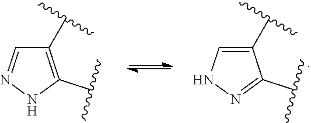 [SMILES: *c1cn[nH]c1* ; *c1c[nH]nc1*]

For another example, compounds including guanidinyl in the ring may under go tautomerism to form a different ring like below:

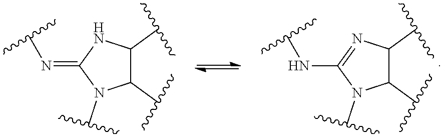 [SMILES: */N=C1\NC(*)C(*)N1* ; *NC1=NC(*)C(*)N1*]

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“deuterated analog” refers to a derivative of an active agent that an arbitrary hydrogen is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “treated”, “treating” or “treatment” as used herein also generally refers to the acquisition of the desired pharmacological and/or physiological effect. The effect may be prophylactic according to the prevention of the disease or its symptoms in whole or in part; and/or may be therapeutic according to the partial or complete stabilization or cure of the disease and/or the side effect due to the disease. As used herein, “treated”, “treating” or “treatment” encompasses any treatment for the disease of a patient, including: (a) prevention of the disease or condition in the patient that may be predisposed to the disease or condition but has not yet been diagnosed; (b) inhibition of the symptoms of the disease, i.e., preventing its development; or (c) remission of the symptoms of the disease, i.e., causing regression of the disease or symptoms in whole or in part.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” or “Cₙ-Cₘ” indicates a range which includes the endpoints, wherein n and m are integers and indicate the number of carbons. Examples include C₁₋₈, C₁₋₆, C₁-C₈, C₁-C₆ and the like.

Unless otherwise specified, the percentages, proportions, ratios or parts used in the present application are by weight or volume. The amount used in the present application is a weight or volume amount. It can be determined easily by those skilled in the art.

Hereinafter, the present application will demonstrate the beneficial effects of the present application by way of examples. Those skilled in the art will recognize that these examples are illustrative and not restrictive. These examples will do not limit the scope of the present application in any way. The experimental methods described in the following examples, unless otherwise specified, are conventional methods; the reagents and materials, unless otherwise specified, are commercially available.

Unless specifically defined elsewhere in this document, all other technical and scientific terms used herein have the meaning commonly understood by one of ordinary skill in the art to which this invention belongs.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise.

Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR

spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.10% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 m1, at room temperature and UV Detection at 214 nm and 254 nm.

In the following examples, the abbreviations below are used:

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| Aq | Aqueous |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| BTEAC | Benzyltriethylammonium chloride |
| C: 40691-33-6 | dichlorobis(tri-o-tolylphosphine)palladium(II) |
| Cbz | Benzyloxycarbonyl |
| DCM | Dichloromethane |
| Con. | Concentrated |
| DavePhos | 2′-(Dicyclohexylphosphino)-N,N-dimethyl-2-biphenylamine |
| DBU | 1,8-diazabicyclo[5.4.0]undec-7-ene |
| DCE | dichloroethane |
| DHP | 3,4-Dihydro-2H-pyran |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h or hr | Hour |
| HATU | 2-(7-Azabenzotriazol-1-yl)-N,N,N′,N′-tetramethyluronium |
| | hexafluorophosphate |
| HBTU | O-(7-Benzotriazole-1-yl)-N, N,N′,N′-tetramethyluronium |
| | hexafluorophosphate |
| Hex | Hexane |
| HPLC | High Performance Liquid Chromatography |
| hrs | hours |
| IBX | 2-Iodoxybenzoic acid |
| IPA | 2-propanol |
| i-PrOH | Isopropyl alcohol |
| KHMDS | Potassium bis(trimethylsilyl)amide |
| LIHMDS | Lithium bis(trimethylsilyl)amide |
| KOAc or AcOK | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]di-chloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride;Sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCl | tert-Butyldimethylsilyl chloride |
| Ti(OiPr)4 | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH, Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |
| TCFH | N,N,N′,N′-Tetramethylchloroformamidinium |
| | hexafluorophosphate |
| CDI | 1,1′-Carbonyldiimidazole |
| aq. | Aqueous |
| DCE | Dichloroethane |
| DEA | N,N-Diethylaniline |
| DIAD | Diisopropyl azodicarboxylate |
| DMA | N,N-Dimethylacetamide |
| DMC | Dimethyl carbonate |
| LCMS | Liquid Chromatography Mass Spectrometry |
| LDA | Lithium diisopropylamide |
| NMR | Nuclear Magnetic Resonance |
| dtbpf | 1,1′-Bis(di-tert-butylphosphino)ferrocene |
| Pd(dtbpf)Cl₂ | [1,1′-Bis(di-tert-butylphosphino)ferrocene]dichloropalladium(II) |
| PPh₃ | triphenylphosphine |
| PPh₃O | Triphenylphosphine oxide |
| Prep-HPLC | Preparative-scale High-Performance Liquid Chromatography |
| RuPhos | 2-Dicyclohexylphosphino-2′,6′-diisopropoxy-1,1′-biphenyl |
| RuPhos Pd G3 | Methanesulfonato(2-dicyclohexylphosphino-2′,6′-di-isopropoxy- |
| | 1,1′-biphenyl)(2′-amino-1,1′-biphenyl-2-yl)palladium(II) |
| Sat. | Saturated |
| T₃P | Propylphosphonic anhydride |
| Pd-PEPPSI-IPentCl | [1,3-bis[2,6-bis(1-propylbutyl)phenyl]-4,5-dichloro-imidazol-2- |
| | ylidene]-dichloro-(3-chloropyridin-1-ium-1-yl)palladium |
| Johnphos | (2-Biphenyl)di-tert-butylphosphine |

### Intermediate 1: (7¹s,7³s,E)-5⁶-bromo-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

### Step 1: methyl (1s,3s)-3-formylcyclobutane-1-carboxylate

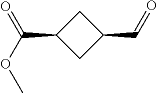

To a stirred solution of methyl 3-(hydroxymethyl)cyclobutane-1-carboxylate (100 g, 693 mmol) in DCM (1000 mL) were added Dess-Martin periodinane (353 g, 832 mmol) at room temperature. The resulting mixture was stirred for 2 h at room temperature. Then the mixture was diluted with DCM (1000 mL), washed with sat. aq. NaHCO₃ (3×1000 mL) and brine (1000 mL). The organic layer was dried over anhydrous Na₂SO₄, filtered and concentrated under reduced pressure to give the crude product as a mixture of cis and trans isomers. The residue was purified by silica gel columnm chromatography, eluted with PE/EtOAc (100:12) to afford the title compound (cis) (45 g, 45.6%), and trans product (30 g, 30.4%); ¹H NMR (500 MHz, CDCl₃) δ 9.70 (s, 1H), 3.69 (s, 3H), 3.23-3.07 (m, 2H), 2.57-2.51 (m, 2H), 2.45-2.39 (in, 2H).

### Step 2: methyl (1s,3s)-3-(((tert-butylsulfinyl)amino)methyl)cyclobutane-1-carboxylate

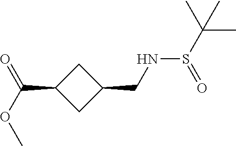 [SMILES: COC(=O)[C@H]1C[C@@H](CNS(=O)C(C)(C)C)C1]

To a stirred solution of methyl (1s,3s)-3-formylcyclobutane-1-carboxylate (45 g, 318 mmol) in DCM (200 mL) were added 2-methylpropane-2-sulfinamide (42.2 g, 348 mmol) and 5 mL. AcOH at room temperature. The resulting mixture was stirred for 16 h at room temperature. The resulting mixture was concentrated and diluted with MeOH (200 mL). Then NaBH₄ (24 g, 633 mmol) was added and the mixture was stirred at room temperature for 4 h. The mixture was concentrated and diluted with DCM (1000 mL), and washed with brine (500 mL×2). The organic layer was dried over anhydrous Na₂SO₄, filtered and concentrated under reduced pressure. The residue was purified by silica gel column chromatography, eluted with PE/EtOAc (4:1) to afford the product (48 g, 61.3%); [M+H]⁺=248.2.

### Step 3: methyl (1s,3s)-3-(aminomethyl)cyclobutane-1-carboxylate hydrochloride

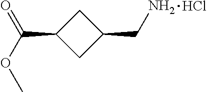

A solution of methyl (1s,3s)-3-(((tert-butylsulfinyl)amino)methyl)cyclobutane-1-carboxylate (48 g, 194 mmol) in 100 mL HCl (4N in dioxane) was stirred for 4 h at room temperature. The resulting mixture was concentrated to afford the product (38 g) as a hydrochloride; [M+H]⁺=144.1.

### Step 4: methyl (1s,3s)-3-(((5-bromo-2-nitrophenyl)amino)methyl)cyclobutane-1-carboxylate

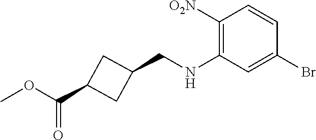 [SMILES: COC(=O)[C@H]1C[C@@H](CNc2cc(Br)ccc2[N+](=O)[O-])C1]

A solution of methyl (1s,3s)-3-(aminomethyl)cyclobutane-1-carboxylate hydrochloride (38 g, 265 mmol), and 4-bromo-2-fluoro-1-nitrobenzene (87.6 g, 398 mmol) and DIEA (137 g, 1.06 mol) in MeCN (500 mL) was stirred for 16 h at 80° C. Then the mixture was concentrated and diluted with DCM (1000 mL), and then washed with sat. aq. NH₄Cl (3×500 mL) and brine (500 mL). The organic layer was dried over anhydrous Na₂SO₄, filtered and concentrated under reduced pressure. The residue was purified by silica gel column chromatography, eluted with EtOAc/PE (0-20%) to afford the product (45 g, 49.4%); [M+H]⁺=343.2.

### Step 5: ((1s,3s)-3-(((5-bromo-2-nitrophenyl)amino)methyl)cyclobutyl)methanol

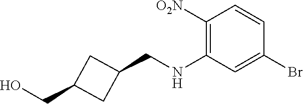 [SMILES: O=[N+]([O-])c1ccc(Br)cc1NC[C@H]1C[C@@H](CO)C1]

A solution of methyl (1s,3s)-3-(((5-bromo-2-nitrophenyl)amino)methyl)cyclobutane-1-carboxylate (45 g, 131 mmol) in 600 mL THF was cooled to −20° C. LiA1H₄ (2.4 M in THF, 60 mL) was added for 15 mins at −20° C. The resulting mixture was stirred at −20° C. for 1 hr. Then the mixture was diluted with EA (600 mL), washed with sat. aq. NH₄Cl (3×500 mL) and brine (500 mL). The organic layer was dried over anhydrous Na₂SO₄, filtered and concentrated under reduced pressure. The residue was purified by silica gel column chromatography, eluted with EtOAc/PE (0-60%) to afford the product (37 g, 89.5%); [M+H]⁺=315.1.

### Step 6: methyl 2-(5-(((1s,3s)-3-(((5-bromo-2-nitrophenyl)amino)methyl)cyclobutyl)methoxy)-1-methyl-1H-pyrazol-4-yl)-6-methylisonicotinate

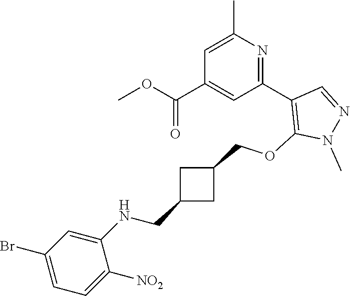 [SMILES: COC(=O)c1cc(C)nc(-c2cnn(C)c2OC[C@H]2C[C@@H](CNc3cc(Br)ccc3[N+](=O)[O-])C2)c1]

To a stirred solution of ((1s,3s)-3-(((5-bromo-2-nitrophenyl)amino)methyl)cyclobutyl)methanol (37 g, 117 mmol), methyl 2-(5-hydroxy-1-methyl-1H-pyrazol-4-yl)-6-methylisonicotinate (27.4 g, 117 mmol), and PPh₃ (37 g, 141 mmol) in THF (500 mL) was added DIAD (28.5 g, 141 mmol). Then the mixture was stirred for 2 h at room temperature. Then the mixture was concentrated and purified by silica gel column chromatography, eluted with EtOAc/PE (0-80%) to afford the product mixed with PPh₃O (72 g); [M+H]⁺=544.3.

### Step 7: methyl 2-(5-(((1s,3s)-3-(((2-amino-5-bromophenyl)amino)methyl)cyclobutyl)methoxy)-1-methyl-1H-pyrazol-4-yl)-6-methylisonicotinate

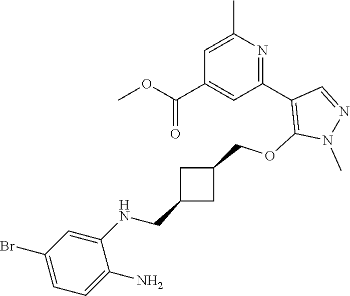 [SMILES: COC(=O)c1cc(C)nc(-c2cnn(C)c2OC[C@H]2C[C@@H](CNc3cc(Br)ccc3N)C2)c1]

To a stirred solution of methyl 2-(5-(((1s,3s)-3-(((5-bromo-2-nitrophenyl)amino)methyl)cyclobutyl)methoxy)-1-methyl-1H-pyrazol-4-yl)-6-methylisonicotinate (72 g, 132 mmol) in THF (1000 mL) was added Raney nickel (39 g, 661 mmol). The resulting mixture was stirred under hydrogen atmosphere (1 atm) at room temperature for 2 hr. Then the mixture was filtered and the filtrate was concentrated. The resulting mixture (66 g) was used for next step without purification; [M+H]⁺=514.3.

### Step 8: methyl 2-(5-(((1s,3s)-3-((6-bromo-2-imino-2,3-dihydro-1H-benzo[d]imidazol-1-yl)methyl)cyclobutyl)methoxy)-1-methyl-1H-pyrazol-4-yl)-6-methylisonicotinate

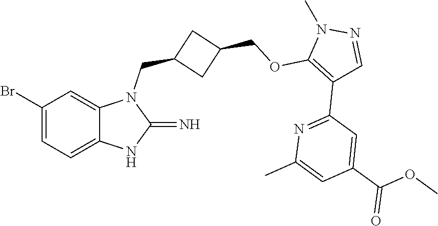 [SMILES: COC(=O)c1cc(C)nc(-c2cnn(C)c2OC[C@H]2C[C@@H](Cn3c(=N)[nH]c4ccc(Br)cc43)C2)c1]

A solution of methyl 2-(5-(((1s,3s)-3-(((2-amino-5-bromophenyl)amino)methyl) cyclobutyl)methoxy)-1-methyl-1H-pyrazol-4-yl)-6-methylisonicotinate (66 g, 128 mmol) and BrCN (41 g, 384 mmol) in MeOH (200 mL) was stirred for 4 h at room temperature. Then the mixture was concentrated and diluted with DCM (1000 mL), and then washed with sat. aq. NaHCO₃ (3×500 mL) and brine (500 mL). The organic layer was dried over anhydrous Na₂SO₄, filtered and concentrated under reduced pressure. The residue was purified by silica gel column chromatography, eluted with MeOH/DCM (0-15%) to afford the product (37 g, 43.5%); [M+H]⁺=539.4.

### Step 9: (7¹s,7³s,E)-5⁶-bromo-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

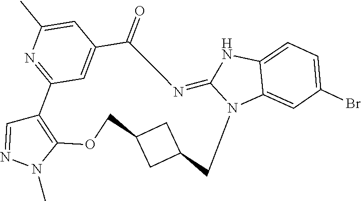 [SMILES: Cc1cc2cc(n1)-c1cnn(C)c1OC[C@H]1C[C@@H](CN3/C(=N/C2=O)Nc2ccc(Br)cc23)C1]

To a stirred solution of methyl 2-(5-(((1s,3s)-3-((6-bromo-2-imino-2,3-dihydro-1H-benzo[d]imidazol-1-yl)methyl)cyclobutyl)methoxy)-1-methyl-1H-pyrazol-4-yl)-6-methylisonicotinate (37 g, 69.6 mmol) in 600 mL THF was added LiHMDS (1N in THF, 137 mL) at room temperature for 15 min. The resulting mixture was stirred at room temperature for 1 hr. Then the mixture was diluted with EA (600 mL), washed with sat. aq. NH₄Cl (2×500 mL) and brine (500 mL). The organic layer was dried over anhydrous Na₂SO₄, filtered and concentrated under reduced pressure. The residue was suspended in 100 mL EA and stirred at room temperature for 1 h. Then the mixture was filtered to afford the product (26 g, 74.7%); [M+H]⁺=507.2.

### Intermediate 2: (7¹r,7³r,E)-5⁶-bromo-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

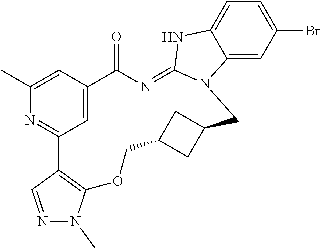 [SMILES: Cc1cc2cc(n1)-c1cnn(C)c1OC[C@H]1C[C@H](CN3/C(=N/C2=O)Nc2ccc(Br)cc23)C1]

The titled compound was prepared in a manner similar to that in intermediate 1, [M+H]⁺=507.2.

### Intermediate 3: (R)-1-(4-(2,6-dioxopiperidin-3-yl)-3,5-difluorophenyl)azetidine-3-carboxylic acid

### Step 1: methyl 1-(4-(2,6-bis(benzyloxy)pyridin-3-yl)-3,5-difluorophenyl)azetidine-3-carboxylate

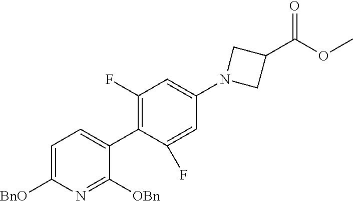 [SMILES: COC(=O)C1CN(c2cc(F)c(-c3ccc(OCc4ccccc4)nc3OCc3ccccc3)c(F)c2)C1]

A mixture of intermediate 16 (3.00 g, 6.22 mmol), methyl azetidine-3-carboxylate hydrochloride (1.41 g, 9.33 mmol), Cs₂CO₃ (6.06 g, 18.66 mmol) and RuPhos Pd G3 (520.7 mg, 0.62 mmol) in toluene (50 mL) was stirred overnight at 100° C. under nitrogen atmosphere. The resulting mixture was diluted with brine (300 mL) and extracted with EtOAc (100 mL×3). The combined organic layers were washed with brine, dried over anhydrous Na₂SO₄. After filtration, the filtrate was concentrated under reduced pressure. The residue was purified by silica gel column chromatography, eluted with PE/EtOAc (2:1) to afford the product (1.7 g, 53%). [M+1]⁺=517.1.

### Step 2: 1-(4-(2,6-bis(benzyloxy)pyridin-3-yl)-3,5-difluorophenyl)azetidine-3-carboxylic acid

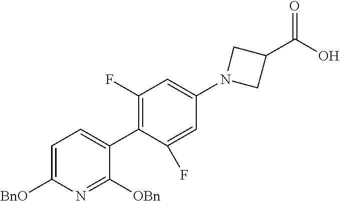 [SMILES: O=C(O)C1CN(c2cc(F)c(-c3ccc(OCc4ccccc4)nc3OCc3ccccc3)c(F)c2)C1]

To a stirred mixture of methyl 1-(4-(2,6-bis(benzyloxy)pyridin-3-yl)-3,5-difluorophenyl)azetidine-3-carboxylate (1.7 g, 3.29 mmol) in THF (20 mL) was added LiOH·H₂O (168 mg, 4 mmol) in 10 mL water dropwise at room temperature. Then the mixture was stirred for 2 hours. The resulting mixture was concentrated in vacuum. The water layer was adjusted pH<5 with 1N HCl and then extracted with EtOAc (3×40 mL). The combined organic layers were washed with brine, dried over anhydrous Na₂SO₄. After filtration, the filtrate was concentrated under reduced pressure to afford the product (1.4 g, 85%), which was used in next step without further purification. [M+1]⁺=503.2.

### Step 3: (R)-1-(4-(2,6-dioxopiperidin-3-yl)-3,5-difluorophenyl)azetidine-3-carboxylic acid

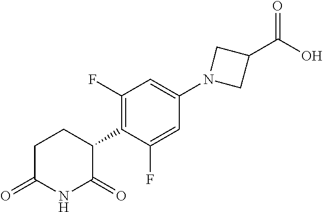 [SMILES: O=C1CC[C@H](c2c(F)cc(N3CC(C(=O)O)C3)cc2F)C(=O)N1]

To a solution of 1-(4-(2,6-bis(benzyloxy)pyridin-3-yl)-3,5-difluorophenyl)azetidine-3-carboxylic acid (1.40 g, 2.79 mmol) in iPrOH (20 mL) and DCM (20 mL) was added Pd/C (1.0 g, 10% wt), and the mixture was stirred at room temperature under hydrogen atmosphere for 48 hours. The resulting mixture was filtered, the filter cake was washed with MeOH (20 mL). The filtrate was concentrated under reduced pressure and purified by SFC (CHIRALPAK AD-3 3.0*100 mm, 3 pm, MeOH (0.1% DEA)), and the title compound corresponded to peak A @) 1.849 min/254 nm (190 mg, 22%). [M+1]⁺=325.3.

### Intermediate 4: (S)-1-(4-((R)-2,6-dioxopiperidin-3-yl)-3,5-difluorophenyl)-4,4-dimethylpyrrolidine-3-carboxylic acid

### Step 1: methyl (S)-4,4-dimethylpyrrolidine-3-carboxylate

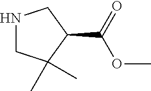

To a stirred solution of (S)-4,4-dimethylpyrrolidine-3-carboxylic acid (2 g, 13.96 mmol) in MeOH (30 mL) was added SOCl₂ (1.66 g, 13.96 mmol) dropwise at 0° C. under nitrogen atmosphere. The resulting mixture was stirred for 2 hrs at 60° C. temperature. The resulting mixture was concentrated under reduced pressure to afford the product (2.1 g, 95.8%) which was used for next step without further purification. [M+H]⁺=158.1

### Steps 2-4: (S)-1-(4-((R)-2,6-dioxopiperidin-3-yl)-3,5-difluorophenyl)-4,4-dimethylpyrrolidine-3-carboxylic acid

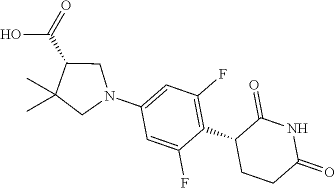 [SMILES: CC1(C)CN(c2cc(F)c([C@H]3CCC(=O)NC3=O)c(F)c2)C[C@H]1C(=O)O]

The titled compound was prepared in a manner similar to that in intermediate 3 synthesis (steps 1-3). [M+H]⁺=367.4.

### Intermediate 5: (R)-2-(4-(2,6-dioxopiperidin-3-yl)-3,5-difluorophenyl)acetaldehyde

### Step 1: ethyl 4-(4-bromo-2,6-difluorophenyl)-4-cyanobutanoate

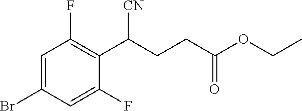 [SMILES: CCOC(=O)CCC(C#N)c1c(F)cc(Br)cc1F]

To a solution of 2-(4-bromo-2,6-difluorophenyl)acetonitrile (10 g, 43.1 mmol) in THE (150 mL) was added LDA (2 M in THF, 24 mL, 48 mmol) dropwise in 20 min at −65° C., the reaction solution was stirred for 1 hour at this temperature, then to this was added ethyl 3-bromopropanoate (9.4 g, 51.7 mmol) in THE (30 mL) dropwise in 10 min. The resulting solution was stirred for 30 min at −65° C., and then allowed to warm to room temperature naturally. The reaction was quenched by the addition of sat. aq. NH₄Cl (50 mL), and extracted with EtOAc (100 mL×3). The combined organic layers were washed with brine, dried over anhydrous Na₂SO₄. After filtration, the filtrate was concentrated under reduced pressure to afford the product (13.8 g, 96.5%). [M+H]⁺=332.0.

### Step 2: 4-(4-bromo-2,6-difluorophenyl)-4-cyanobutanoic acid

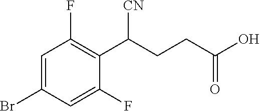 [SMILES: N#CC(CCC(=O)O)c1c(F)cc(Br)cc1F]

To a solution of ethyl 4-(4-bromo-2,6-difluorophenyl)-4-cyanobutanoate (13.5 g, 40.7 mmol) in THF/H₂O (90 mL/30 mL) was added LiOH (2.9 g, 0.122 mol). The reaction mixture was stirred for 12 h at room temperature. The resulting mixture was diluted with water, and extracted with EtOAc (50 mL×2). The pH value of water phase was adjusted to 4-5 with 1 N HCl (10 mL), and extracted with EtOAc (50 mL×3). The combined organic layers were washed with brine (50 mL×3), and dried over anhydrous Na₂SO₄. After filtration, the filtrate was concentrated under reduced pressure to afford the product (10.2 g, 82.5%). [M+H]⁺=304.2.

### Step 3: 3-(4-bromo-2,6-difluorophenyl)piperidine-2,6-dione

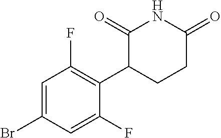 [SMILES: O=C1CCC(c2c(F)cc(Br)cc2F)C(=O)N1]

To a stirred solution of 4-(4-bromo-2,6-difluorophenyl)-4-cyanobutanoic acid (10.2 g, 33.5 mmol) in toluene (100 mL) was added cone. H₂SO₄ (2 mL, 36.9 mmol). The resulting solution was stirred at 100 TC for 3 h. The reaction mixture was concentrated under vacuum, then the mixture was poured into water. The pH value was adjusted to 7-8 with sat. aq. NaHCO₃ (40 mL), and resulting solution was extracted with EtOAc (50 mL×3). The combined organic layers were washed with water and brine, dried over anhydrous Na₂SO₄, filtered and concentrated to afford the product (8.2 g, 80.4%). [M+H]⁺=304.3.

### Step 4: (R,E)-3-(4-(2-ethoxyvinyl)-2,6-difluorophenyl)piperidine-2,6-dione

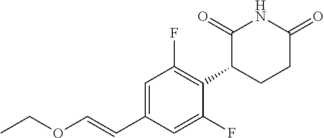 [SMILES: CCO/C=C/c1cc(F)c([C@H]2CCC(=O)NC2=O)c(F)c1]

To a stirred solution of 3-(4-bromo-2,6-difluorophenyl)piperidine-2,6-dione (8.2 g, 27.0 mmol) and (E)-2-(2-ethoxyvinyl)-4,4,5,5-tetramethyl-1,3,2-dioxaborolane (6.4 g, 32.4 mmol) in DMF/H₂O (100 mL/20 mL) were added Pd(dtbpf) Cl₂ (883 mg, 1.35 mmol) and CsF (8.2 g, 54.0 mmol). The resulting mixture was stirred for 2 h at 80° C. under nitrogen atmosphere. The reaction solution was diluted with water, extracted with EtOAc (100 mL×2). The combined organic layers were washed with water and brine, dried over anhydrous Na₂SO₄, filtered and concentrated under reduced pressure. The residue was purified by SFC (IH (3*25 cm, 5 um), 13% EtOH/87% CO₂, 100 bar, 100 ml/min) and the title compound corresponded to peak A @1.679 min/254 nm. (3.1 g, 39.0%). [M+H]⁺=296.1.

### Step 5: (R)-2-(4-(2,6-dioxopiperidin-3-yl)-3,5-difluorophenyl)acetaldehyde

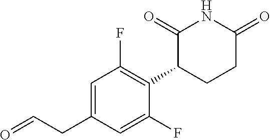 [SMILES: O=CCc1cc(F)c([C@H]2CCC(=O)NC2=O)c(F)c1]

(R,E)-3-(4-(2-ethoxyvinyl)-2,6-difluorophenyl)piperidine-2,6-dione (3.1 g, 10.4 mmol) was dissolved in FA (50 mL). The resulting solution was stirred for 2 h at room temperature. The reaction solution was evaporated to dryness to afford the product (2.6 g, 91.8%). [M+H]⁺=268.1.

### Intermediate 6: (R)-3-(2,6-difluoro-4-(4-oxopiperidin-1-yl)phenyl)piperidine-2,6-dione Step 1: 8-(4-(2,6-bis(benzyloxy)pyridin-3-yl)-3,5-difluorophenyl)-1,4-dioxa-8-azaspiro[4.5]decane

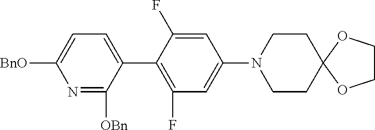 [SMILES: Fc1cc(N2CCC3(CC2)OCCO3)cc(F)c1-c1ccc(OCc2ccccc2)nc1OCc1ccccc1]

To the solution of intermediate 16 (30 g, 62.24 mmol), 1,4-dioxa-8-azaspiro[4.5]decane (10.68 g, 74.69 mmol) and Cs₂CO₃ (40.58 g, 124.48 mmol) in 500 mL dioxane, Pd₂(dba)₃ (2.85 g, 3.11 mmol) and Xantphos (3.6 g, 6.22 mmol) was added at N₂ atmosphere. The mixture was stirred at 80° C. for 16 hours under N₂ protected. The mixture was diluted with EtOAc and filtered. The filtrated was concentrated in vacuum and purified by silica column chromatography (EA:PE=0-80%) to afford the crude product. The crude was recrystallized with MeOH and filtered. The filter cake was dried to afford the title compound (26.8 g, 79% yield); [M+H]⁺=544.9.

### Step 2: 3-(2,6-difluoro-4-(1,4-dioxa-8-azaspiro[4.5]decan-8-yl)phenyl)piperidine-2,6-dione

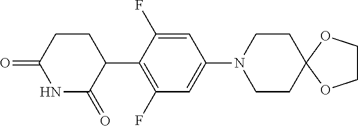 [SMILES: O=C1CCC(c2c(F)cc(N3CCC4(CC3)OCCO4)cc2F)C(=O)N1]

To the solution of 8-(4-(2,6-bis(benzyloxy)pyridin-3-yl)-3,5-difluorophenyl)-1,4-dioxa-8-azaspiro[4.5]decane (26.8 g, 49.26 mmol) in 400 mL DMF and 80 mL iPrOH, Pd/C (27 g, 10 wt. %, wet) was added. The mixture was stirred at 45° C. for 16 hours at H₂ atmosphere (4 bar). The mixture was filtered and the filter cake was washed with DMF. The combined liquid was concentrated in vacuum to afford the title compound (15 g, 83.2% yield); [M+H]⁺=367.1.

### Step 3: (R)-3-(2,6-difluoro-4-(1,4-dioxa-8-azaspiro[4.5]decan-8-yl)phenyl)piperidine-2,6-dione

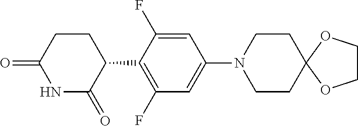 [SMILES: O=C1CC[C@H](c2c(F)cc(N3CCC4(CC3)OCCO4)cc2F)C(=O)N1]

The title compound was purified by chiral-HPLC (CHIRALPAK IF (2*25 cm, 5 um), MtBE(0.1% DEA):(MeOH:DCM=1:1)=50:50, 100 bar, 20 ml/min) (4.47 g, 37% yield from 12 g racemate); [M+H]⁺=367.1.

The title compound could also be purified by chiral-HPLC (CHIRALPAK IF (4.6*50 mm, 3 um), MtBE (0.1% DEA):(MeOH:DCM=1:1)=50:50, 1 ml/min) and the title compound corresponded to peak A @0.990 min/254 nm.

### Step 4: (R)-3-(2,6-difluoro-4-(4-oxopiperidin-1-yl)phenyl)piperidine-2,6-dione

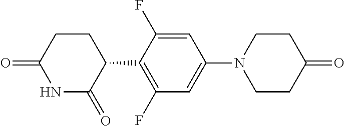 [SMILES: O=C1CCN(c2cc(F)c([C@H]3CCC(=O)NC3=O)c(F)c2)CC1]

(R)-3-(2,6-difluoro-4-(1,4-dioxa-8-azaspiro[4.5]decan-8-yl)phenyl)piperidine-2,6-dione (1 g, 2.73 mmol) was placed in 100 mL round bottom flask with a magnetic stir bar. Then, 10 mL 12N HCl aqueous was added. The mixture was stirred at room temperature for 30 minutes. The mixture was added dropwise to sat. aq. NaHCO₃ solution and finally pH=6-7. The liquid was extracted with DCM and separated. The organic phase was concentrated in vacuum and purified with silica gel column chromatography (MeOH:DCM=0-5%) to afford the title compound (850 mg, 96.7% yield); [M+H]⁺=323.1.

### Intermediate 7: (R)-3-(2,6-difluoro-4-(3-oxoazetidin-1-yl)phenyl)piperidine-2,6-dione

### Step 1: 2,6-bis(benzyloxy)-3-(4-(3-(benzyloxy)azetidin-1-yl)-2,6-difluorophenyl)pyridine

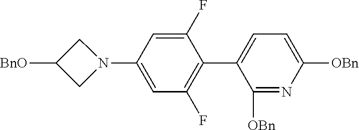 [SMILES: Fc1cc(N2CC(OCc3ccccc3)C2)cc(F)c1-c1ccc(OCc2ccccc2)nc1OCc1ccccc1]

A solution of intermediate 16 (20 g, 41.49 mmol), 3-(benzyloxy)azetidine hydrogen chloride (9.96 g, 49.79 mmol), Pd₂(dba)₃ (3.79 g, 4.15 mmol), RuPhos (3.88 g, 8.3 mmol) and Cs₂CO₃ (40.58 g, 124.47 mmol) in dioxane (400 mL) was stirred at 100° C. for 3 h under nitrogen atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (10:1) to afford 2,6-bis(benzyloxy)-3-(4-(3-(benzyloxy)azetidin-1-yl)-2,6-difluorophenyl)pyridine (17 g, 72.6%). [M+H]⁺=565.6.

### Step 2: 3-(2,6-difluoro-4-(3-hydroxyazetidin-1-yl)phenyl)piperidine-2,6-dione

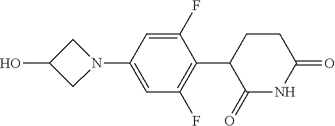 [SMILES: O=C1CCC(c2c(F)cc(N3CC(O)C3)cc2F)C(=O)N1]

To a solution of 2,6-bis(benzyloxy)-3-(4-(3-(benzyloxy)azetidin-1-yl)-2,6-difluorophenyl)pyridine (17 g, 30.09 mmol) in DCM/THF (200 mL/200 mL), was added Pd/C (10 wt %, wet, 34 g). The mixture was stirred for 2 days at 65° C. under hydrogen atmosphere. After cooled to room temperature. The resulting mixture was filtered, the filter cake was washed with IPA. The filtrate was concentrated under reduced pressure and purified by trituration with DCM/MeOH (10/1) to afford 3-(2,6-difluoro-4-(3-hydroxyazetidin-1-yl)phenyl)piperidine-2,6-dione (8 g, 89.9%). [M+H]⁺=297.1

### Step 3: 3-(4-(3-((tert-butyldimethylsilyl)oxy)azetidin-1-yl)-2,6-difluorophenyl)piperidine-2,6-dione

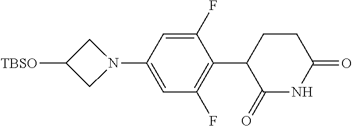 [SMILES: CC(C)(C)[Si](C)(C)OC1CN(c2cc(F)c(C3CCC(=O)NC3=O)c(F)c2)C1]

The solution of 3-(2,6-difluoro-4-(3-hydroxyazetidin-1-yl)phenyl)piperidine-2,6-dione (11 g, 37.16 mmol), TBSCl (11.15 g, 74.32 mmol) and imidazole (7.58 g, 111.49 mmol) in DMF (200 mL) was stirred for 2 h at rt. The resulting mixture was diluted with sat. aq. NaHCO₃ (500 ml) and extracted with EA (200 mL×3). The combined organic layers were washed with brine, dried over anhydrous Na₂SO₄. After filtration, the filtrate was concentrated under reduced pressure. The residue was purified by silica gel column chromatography, eluted with PE/EA (10:1) to afford 3-(4-(3-((tert-butyldimethylsilyl)oxy)azetidin-1-yl)-2,6-difluorophenyl)piperidine-2,6-dione (11 g, 72.4%). [M+H]⁺=411.2

### Step 4: (R)-3-(4-(3-((tert-butyldimethylsilyl)oxy)azetidin-1-yl)-2,6-difluorophenyl)piperidine-2,6-dione

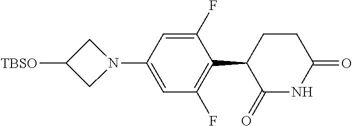 [SMILES: CC(C)(C)[Si](C)(C)OC1CN(c2cc(F)c([C@H]3CCC(=O)NC3=O)c(F)c2)C1]

The crude product (11 g) was purified by Prep-HPLC and the title compound corresponds to (CHIRALPAK IF-3, 4.6*50 mm, 3 μm, MtBE (0.1% DEA):(MeOH:DCM=1:1)=80:20, flow 1.0 mL/min, peak A @1.084 min). (4 g, 36%, ee=100%). [M+H]⁺=411.2

### Step 5: (R)-3-(2,6-difiuoro-4-(3-hydroxyazetidin-1-yl)phenyl)piperidine-2,6-dione

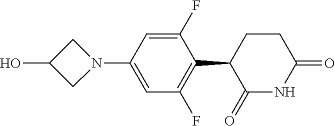 [SMILES: O=C1CC[C@H](c2c(F)cc(N3CC(O)C3)cc2F)C(=O)N1]

(R)-3-(4-(3-((tert-butyldimethylsilyl)oxy)azetidin-1-yl)-2,6-difluorophenyl)piperidine-2,6-dione (2.6 g, 6.3 mmol) was placed in a 250 mL round bottom flask with a magnetic stir bar. Then, 60 mL THF and 60 mL 1M HCl were added at 0° C. The mixture was stirred at room temperature for 3 hours. The mixture was added dropwise to sat. aq. NaHCO₃ and pH was adjusted to 6-7. The liquid was extracted with DCM (50 mL×3) and separated. The combined organic phase was concentrated in vacuum and purified with combiflash (DCM MeOH=20:1) to afford the title compound (1.8 g, 95% yield). [M+H]⁺=297.2.

### Step 6: (R)-3-(2,6-difluoro-4-(3-oxoazetidin-1-yl)phenyl)piperidine-2,6-dione

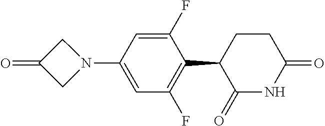 [SMILES: O=C1CN(c2cc(F)c([C@H]3CCC(=O)NC3=O)c(F)c2)C1]

To a stirred solution of (R)-3-(2,6-difluoro-4-(3-hydroxyazetidin-1-yl)phenyl)piperidine-2,6-dione (1.8 g, 6.0 mmol) in DCM (60 mL) were added Dess-Martin periodinane (3.8 g, 9.0 mmol) at room temperature. The resulting mixture was stirred for 2 h at room temperature. Then the mixture was diluted with DCM (100 mL), washed with sat. aq. Na₂S₂O₃ (100 mL), sat. aq. NaHCO₃ (3×100 mL) and brine (100 mL). The organic layer was dried over anhydrous Na₂SO₄, filtered and concentrated under reduced pressure to give the crude product (2.0 g), which was used without further purification. [M+H]⁺=295.2.

### Intermediate 8: (R)-1-(4-(2,6-dioxopiperidin-3-yl)-3,5-difluorophenyl)azetidine-3-carbaldehyde

### Step 1: (R)-3-(2,6-difluoro-4-(3-(hydroxymethyl)azetidin-1-yl)phenyl)piperidine-2,6-dione

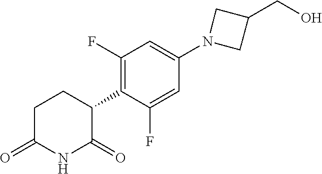 [SMILES: O=C1CC[C@H](c2c(F)cc(N3CC(CO)C3)cc2F)C(=O)N1]

To the solution of intermediate 3 (6.5 g, 20 mmol) in THE was added BH₃·THF (30 mL, 1 M in THF) dropwise at 0° C. The reaction mixture was stirred at room temperature overnight. Then the mixture was quenched with MeOH (20 mL) and diluted with DCM (150 mL), washed with sat. aq. NaHCO₃ (3×100 mL) and brine (100 mL). The organic layer was dried over anhydrous Na₂SO₄, filtered and concentrated under reduced pressure to give the crude product, which was purified by silica gel column chromatography, eluted with DCM/MeOH (30:1 to 15:1) to afford the title product (3.8 g, 61.2%). [M+H]⁺=311.2.

### Step 2: (R)-1-(4-(2,6-dioxopiperidin-3-yl)-3,5-difluorophenyl)azetidine-3-carbaldehyde

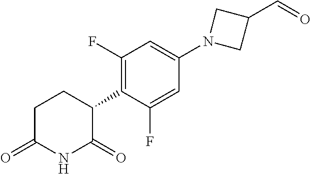 [SMILES: O=CC1CN(c2cc(F)c([C@H]3CCC(=O)NC3=O)c(F)c2)C1]

A mixture of (R)-3-(2,6-difluoro-4-(3-(hydroxymethyl)azetidin-1-yl)phenyl)piperidine-2,6-dione (3.8 g, 12.3 mmol) and IBX (6.8 g, 24.6 mmol) in DMSO (80 mL) was stirred in a flask at room temperature overnight. The reaction was quenched with water and the mixture was extracted with DCM (60 mL×3). The combined organic layers were washed with sat. aq. Na₂S₂O₃ (100 mL), sat. aq. NaHCO₃ (100 mL×2), sat. aq. NaCl (100 mL×2), dried over anhydrous Na₂SO₄, filtered and concentrated under vacuum to afford the product (2.3 g, 70.10%), which was used without further purification. [M+H]⁺309.1.

### Intermediate 9: 3-(3-fluoro-5-(4-oxopiperidin-1-yl)pyridin-2-yl)piperidine-2,6-dione

### Step 1: 8-(6-chloro-5-fluoropyridin-3-yl)-1,4-dioxa-8-azaspiro[4.5]decane

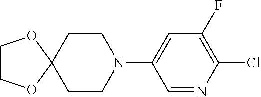 [SMILES: Fc1cc(N2CCC3(CC2)OCCO3)cnc1Cl]

To the solution of 5-bromo-2-chloro-3-fluoropyridine (2.0 g, 9.5 mmol), 1,4-dioxa-8-azaspiro[4.5]decane (2.0 g, 14.25 mmol) and Cs₂CO₃ (9.26 g, 28.5 mmol) in 80 mL 1,4-dioxane were added Pd₂(dba)₃ (871 mg, 0.95 mmol) and XantPhos (880 mg, 1.9 mmol). The mixture was stirred at 100° C. for 3 hours under nitrogen atmosphere. After LCMS showed the reaction was completed, the mixture was filtered through a celite pad and washed with DCM. The filtrate was concentrated under reduced pressure to give the crude residue, which was purified by silica column chromatography (PE:EA=50:1-2:1) to afford the product (1.7 g, 65.8%). [M+H]⁺=273.5.

### Step 2: 8-(2′,6′-bis(benzyloxy)-3-fluoro-[2,3′-bipyridin]-5-yl)-1,4-dioxa-8-azaspiro[4.5]decane

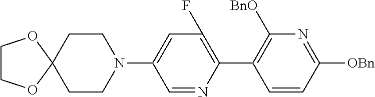 [SMILES: Fc1cc(N2CCC3(CC2)OCCO3)cnc1-c1ccc(OCc2ccccc2)nc1OCc1ccccc1]

To the solution of 8-(6-chloro-5-fluoropyridin-3-yl)-1,4-dioxa-8-azaspiro[4.5]decane (1.6 g, 5.87 mmol), 2,6-bis(benzyloxy)-3-(4,4,5,5-tetramethyl-1,3,2-dioxaborolan-2-yl)pyridine (2.0 g, 7.04 mmol) and K₂CO₃ (2.43 g, 17.61 mmol) in 40 mL 1,4-dioxane and 8 mL H₂O was added Pd(dppf)Cl₂ (858 mg, 1.17 mmol). The mixture was stirred at 100° C. overnight under nitrogen atmosphere. The mixture was filtered through a celite pad and washed with DCM. The filtrate was concentrated under reduced pressure to give the crude residue, which was purified by silica column chromatography (PE:EA=50:1-2:1) to afford the product (2.7 g, 90.1%). [M+H]⁺=528.5.

### Step 3: 3-(3-fluoro-5-(1,4-dioxa-8-azaspiro[4.5]decan-8-yl)pyridin-2-yl)piperidine-2,6-dione

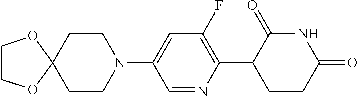 [SMILES: O=C1CCC(c2ncc(N3CCC4(CC3)OCCO4)cc2F)C(=O)N1]

8-(2′,6′-bis(benzyloxy)-3-fluoro-[2,3′-bipyridin]-5-yl)-1,4-dioxa-8-azaspiro[4.5]decane (2.7 g, 5.12 mmol) was dissolved in DMF (40 mL) and iPrOH (20 mL). Pd/C (2.7 g, 10 wt. %, wet) was added to the solution in one portion. The resulting mixture was stirred under hydrogen atmosphere (1 atm) at 50° C. overnight. The solid was filtered off and the filtrate was concentrated to give the crude product. The crude was purified by silica gel column chromatography, eluted with DCM/MeOH (20:1) to afford the product (1.6 g, 89.8%); [M+H]⁺=350.5.

### Step 4: 3-(3-fluoro-5-(4-oxopiperidin-1-yl)pyridin-2-yl)piperidine-2,6-dione

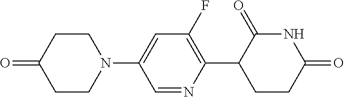 [SMILES: O=C1CCN(c2cnc(C3CCC(=O)NC3=O)c(F)c2)CC1]

3-(3-fluoro-5-(1,4-dioxa-8-azaspiro[4.5]decan-8-yl)pyridin-2-yl)piperidine-2,6-dione (500 mg, 1.43 mmol) was placed in a 25 mL round bottom flask with a magnetic stir bar. Then, 5 mL concentrated HCl aqueous was added. The mixture was stirred at room temperature for 2 hours. The mixture was added dropwise to sat. aq. NaHCO₃ and pH was adjusted to 6-7. The liquid was extracted with DCM (20 mL×3) and separated. The combined organic phase was concentrated in vacuum and purified with combiflash (DCM:MeOH=20:1) to afford the title compound (350 mg, 80.2% yield). [M+H]⁺=306.5.

### Intermediate 10: 3-(4-methyl-5-(4-oxopiperidin-1-yl)pyridin-2-yl)piperidine-2,6-dione

### Step 1: 2′,6′-bis(benzyloxy)-5-bromo-4-methyl-2,3′-bipyridine

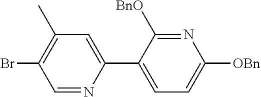 [SMILES: Cc1cc(-c2ccc(OCc3ccccc3)nc2OCc2ccccc2)ncc1Br]

To the solution of 5-bromo-2-iodo-4-methylpyridine (4.8 g, 16.1 mmol), 2,6-bis(benzyloxy)-3-(4,4,5,5-tetramethyl-1,3,2-dioxaborolan-2-yl)pyridine (6.7 g, 16.1 mmol) and K₂CO₃ (4.5 g, 32.2 mmol) in 100 mL 1,4-dioxane and 20 mL H₂O was added Pd(dppf)Cl₂ (1.2 g, 1.61 mmol). The mixture was stirred at 90° C. for 16 hours. The mixture was concentrated under reduced pressure to give the crude residue, which was purified by silica column chromatography (PE:EA=100:1-5:1) to afford the product (6.7 g, 90.5%). [M+H]⁺=461.5.

### Step 2: 8-(2′,6′-bis(benzyloxy)-4-methyl-[2,3′-bipyridin]-5-yl)-1,4-dioxa-8-azaspiro[4.5]decane

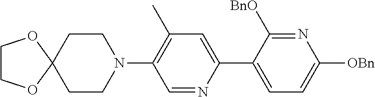 [SMILES: Cc1cc(-c2ccc(OCc3ccccc3)nc2OCc2ccccc2)ncc1N1CCC2(CC1)OCCO2]

To the solution of 2′,6′-bis(benzyloxy)-5-bromo-4-methyl-2,3′-bipyridine (6.7 g, 14.5 mmol), 1,4-dioxa-8-azaspiro[4.5]decane (5.2 g, 36.3 mmol) and Cs₂CO₃ (9.4 g, 29.0 mmol) in 80 mL DMA, were added Pd₂(dba)₃ (2.6 g, 2.9 mmol) and RuPhos (2.7 g, 5.8 mmol). The mixture was stirred at 100° C. for 16 hours under nitrogen atmosphere. The mixture was filtered through a celite pad and washed with DCM. The filtrate was concentrated under reduced pressure to give the crude residue, which was purified by silica column chromatography (PE:EA=50:1-2:1) to afford the product (5.3 g, 69.7%). [M+H]⁺=524.5.

### Step 3: 3-(4-methyl-5-(1,4-dioxa-8-azaspiro[4.5]decan-8-yl)pyridin-2-yl)piperidine-2,6-dione

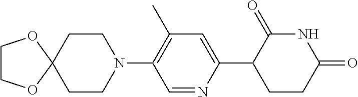 [SMILES: Cc1cc(C2CCC(=O)NC2=O)ncc1N1CCC2(CC1)OCCO2]

To the solution of 8-(2′,6′-bis(benzyloxy)-4-methyl-[2,3′-bipyridin]-5-yl)-1,4-dioxa-8-azaspiro[4.5]decane (5.3 g, 10.1 mmol) in 75 mL DMF and 75 mL iPrOH, Pd/C (2.0 g, 10 wt. %, wet) was added. The mixture was stirred at 50° C. for 20 hours under hydrogen atmosphere (balloon). The mixture was cooled to room temperature and filtered by celite directly. The filtrate was concentrated in vacuum to afford the desired product (2.7 g, 77.1%). [M+H]⁺=346.6.

### Step 4: 3-(4-methyl-5-(4-oxopiperidin-1-yl)pyridin-2-yl)piperidine-2,6-dione

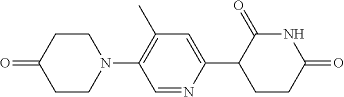 [SMILES: Cc1cc(C2CCC(=O)NC2=O)ncc1N1CCC(=O)CC1]

3-(4-methyl-5-(1,4-dioxa-8-azaspiro[4.5]decan-8-yl)pyridin-2-yl)piperidine-2,6-dione (2.7 g, 7.8 mmol) was placed in 250 mL round bottom flask with a magnetic stir bar. Then 45 mL 8N HCl aqueous was added. The mixture was stirred at room temperature for 2 hours. The mixture was added dropwise to sat. aq. NaHCO₃ and the pH was adjusted to 6-7. The liquid was extracted with DCM (40 mL×3) and separated. The combined organic phase was concentrated in vacuum and purified with combiflash (DCM:MeOH=25:1) to afford the title compound (2.2 g, 93.6% yield). [M+H]⁺=302.5.

### Intermediate 11: 3-(5-(4-oxopiperidin-1-yl)pyridin-2-yl)piperidine-2,6-dione

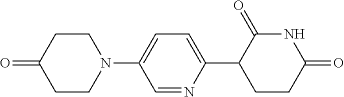 [SMILES: O=C1CCN(c2ccc(C3CCC(=O)NC3=O)nc2)CC1]

The title compound was prepared in a procedure similar to that in intermediate 10. [M+H]⁺=288.2.

### Intermediate 12: 3-(6-methyl-5-(4-oxopiperidin-1-yl)pyridin-2-yl)piperidine-2,6-dione

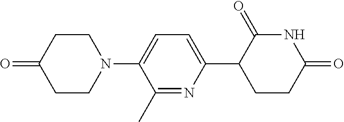 [SMILES: Cc1nc(C2CCC(=O)NC2=O)ccc1N1CCC(=O)CC1]

The title compound was prepared in a procedure similar to that in intermediate 10. [M+H]⁺=302.5.

### Intermediate 13: 2-((4-(2,6-dioxopiperidin-3-yl)-3,5-difluorophenyl)amino)ethyl methanesulfonate

### Step 1: 2-((4-(2,6-bis(benzyloxy)pyridin-3-yl)-3,5-difluorophenyl)amino)ethan-1-ol

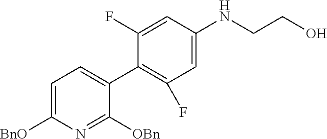 [SMILES: OCCNc1cc(F)c(-c2ccc(OCc3ccccc3)nc2OCc2ccccc2)c(F)c1]

To the solution of intermediate 16 (180 g, 374 mmol), 2-aminoethan-1-ol (45.6 g, 748 mmol) and K₃PO₄ (158.6 g, 748 mmol) in 1 L DMSO, CuI (7.1 g, 37.4 mmol) and L-Proline (4.3 g, 37.4 mmol) was added under N₂ protected. The mixture was stirred at 90° C. for 16 hours. The mixture was diluted with EtOAc and filtered. The filtrate was washed with saturated NaCl aqueous and concentrated in vacuum. The crude was purified with silica column chromatography (EA:PE=0-70%) to afford the title compound (109 g, 63% yield). [M+H]⁺=463.6.

### Step 2: 3-(2,6-difluoro-4-((2-hydroxyethyl)amino)phenyl)piperidine-2,6-dione

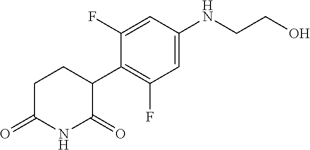 [SMILES: O=C1CCC(c2c(F)cc(NCCO)cc2F)C(=O)N1]

To the solution of 2-((4-(2,6-bis(benzyloxy)pyridin-3-yl)-3,5-difluorophenyl)amino)ethan-1-ol (108 g, 234 mmol) in 200 mL DCM and 1600 mL IPA, Pd/C (108 g, 10% w.t.) was added. The mixture was stirred at 45° C. for 16 hours at H₂ atmosphere. The mixture was filtered by celite. The filter cake was washed with DMF 2 times and the filtrate was combined and concentrated in vacuum to afford the title compound (45.6 g, 68% yield). [M+H]⁺=285.6.

### Step 3: 2-((4-(2,6-dioxopiperidin-3-yl)-3,5-difluorophenyl)amino)ethyl methanesulfonate

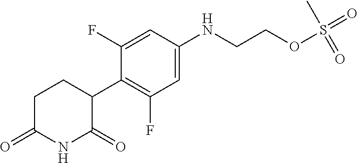 [SMILES: CS(=O)(=O)OCCNc1cc(F)c(C2CCC(=O)NC2=O)c(F)c1]

To the solution of 3-(2,6-difluoro-4-((2-hydroxyethyl)amino)phenyl)piperidine-2,6-dione (5.1 g, 18 mmol) and Et₃N (5.4 g, 54 mmol) in 100 mL DCM, MsCl (4.1 g, 36 mmol) was added. The mixture was stirred at room temperature for 1 hour. After LCMS shown the reaction was completed. The mixture was concentrated in vacuum and purified with silica column chromatography (MeOH:DCM=0-3%) to afford the title compound (4.2 g, 64% yield). [M+H]⁺=363.6.

### Intermediate 14: (R)-3-(2,6-difluoro-4-(piperazin-1-yl)phenyl)piperidine-2,6-dione

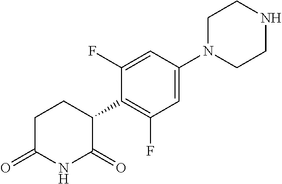 [SMILES: O=C1CC[C@H](c2c(F)cc(N3CCNCC3)cc2F)C(=O)N1]

### Step 1: benzyl 4-(4-(2,6-bis(benzyloxy)pyridin-3-yl)-3,5-difluorophenyl)piperazine-1-carboxylate

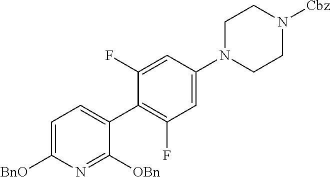 [SMILES: O=C(OCc1ccccc1)N1CCN(c2cc(F)c(-c3ccc(OCc4ccccc4)nc3OCc3ccccc3)c(F)c2)CC1]

To the solution of intermediate 16 (4.8 g, 10 mmol), benzyl piperazine-1-carboxylate (2.4 g, 11 mmol) and Cs₂CO₃ (6.5 g, 20 mmol) in 100 mL dioxane was added Pd₂(dba)₃ (457 mg, 0.5 mmol) and Xantphos (578 mg, 1 mmol). The mixture was stirred at 90° C. for 18 hours under N₂ atmosphere. After LCMS showed the reaction was completed, the mixture was diluted with EtOAc and filtered. The filtrate was concentrated in vacuum and the crude product was purified by silica column chromatography (EA:PE=0-50%) to afford the desired product (5.9 g, 95%). [M+H]⁺=622.7.

### Step 2: (R)-3-(2,6-difluoro-4-(piperazin-1-yl)phenyl)piperidine-2,6-dione

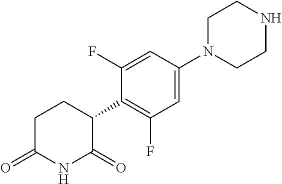 [SMILES: O=C1CC[C@H](c2c(F)cc(N3CCNCC3)cc2F)C(=O)N1]

To the solution of benzyl 4-(4-(2,6-bis(benzyloxy)pyridin-3-yl)-3,5-difluorophenyl)piperazine-1-carboxylate (5.9 g, 9.5 mmol) in 5 mL DCM and 100 mL IPA was added Pd/C (5.9 g, 10% w.t.). The mixture was stirred at 45° C. for 18 hours under H₂ atmosphere. After LCMS showed the reaction was completed, the mixture was filtered through celite. The filter cake was suspended in 50 mL DMF and filtered. The filtrate was combined and concentrated in vacuum. The crude product was washed with MeOH and purified by chiral HPLC (IF (2*25 cm, 5 um), 60% MtBE/40% MeOH:DCM=1:1, 80 bar, 20 ml/min) (2.5 g, 85%). [M+H]⁺=310.7.

The title compound could also be purified by chiral SFC (column: Chiral ND (2) 3.0*100 mm, 3 um; solvent A: CO₂, solvent B: IPA (0.1% DEA), gradient (B %): 10% to 50% in 2.0 min, hold 1.0 min at 50%; flow 2 mL/min, retention time 1.979 min).

### Intermediate 15: Step 3: 3-(6-methyl-5-(piperazin-1-yl)pyridin-2-yl)piperidine-2,6-dione

### Step 1: tert-butyl 4-(2′,6′-bis(benzyloxy)-6-methyl-[2,3′-bipyridin]-5-yl)piperazine-1-carboxylate

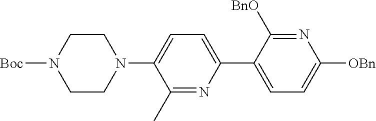 [SMILES: Cc1nc(-c2ccc(OCc3ccccc3)nc2OCc2ccccc2)ccc1N1CCN(C(=O)OC(C)(C)C)CC1]

To the solution of 2′,6′-bis(benzyloxy)-5-bromo-6-methyl-2,3′-bipyridine (4.0 g, 8.7 mmol) (the compound was obtained through the similar method of intermediate 10 step 1), tert-butyl piperazine-1-carboxylate (4.0 g, 17.4 mmol) and Cs₂CO₃ (5.7 g, 17.4 mmol) in 80 mL DMA, were added Pd₂(dba)₃ (1.6 g, 1.74 mmol) and RuPhos (1.6 g, 3.48 mmol). The mixture was stirred at 100° C. for 16 hours under nitrogen atmosphere. The mixture was filtered through a celite pad and washed with DCM. The filtrate was concentrated under reduced pressure to give the crude residue, which was purified by silica column chromatography (PE:EA=50:1-2:1) to afford the product (1.9 g, 38.8%). [M+H]⁺=567.5.

### Step 2: tert-butyl 4-(6-(2,6-dioxopiperidin-3-yl)-2-methylpyridin-3-yl)piperazine-1-carboxylate

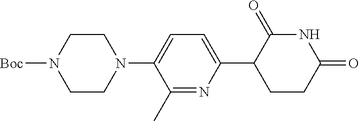 [SMILES: Cc1nc(C2CCC(=O)NC2=O)ccc1N1CCN(C(=O)OC(C)(C)C)CC1]

To the solution of tert-butyl 4-(2′,6′-bis(benzyloxy)-6-methyl-[2,3′-bipyridin]-5-yl)piperazine-1-carboxylate (1.9 g, 3.7 mmol) in 25 mL DMF and 25 mL iPrOH was added Pd/C (1.2 g, 10 wt. %, wet). The mixture was stirred at 50° C. for 16 hours under hydrogen atmosphere (balloon). The mixture was cooled to room temperature and filtered by celite directly. The filtrate was concentrated in vacuum to afford the desired product (1.2 g, 92.3%). [M+H]⁺=389.5.

### Step 3: 3-(6-methyl-5-(piperazin-1-yl)pyridin-2-yl)piperidine-2,6-dione

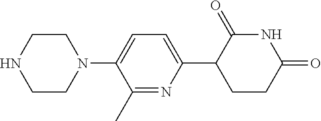 [SMILES: Cc1nc(C2CCC(=O)NC2=O)ccc1N1CCNCC1]

To a 50-mL round-bottomed flask equipped with a magnetic stir bar was added tert-butyl 4-(6-(2,6-dioxopiperidin-3-yl)-2-methylpyridin-3-yl)piperazine-1-carboxylate (400 mg, 1.0 mmol) and HCl/1,4-dioxane (10 mL). After stirring at room temperature overnight, the reaction mixture was concentrated under reduced pressure to afford the product (as a hydrochloride) (350 mg), which was used without further purification. [M+H]⁺=289.5.

### Intermediate 16: 2,6-bis(benzyloxy)-3-(4-bromo-2,6-difluorophenyl)pyridine

### Step 1: 2,6-bis(benzyloxy)-3-(4,4,5,5-tetramethyl-1,3,2-dioxaborolan-2-yl)pyridine

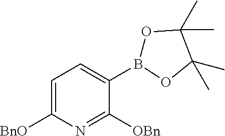 [SMILES: CC1(C)OB(c2ccc(OCc3ccccc3)nc2OCc2ccccc2)OC1(C)C]

4 reactions were carried out in parallel.

To a solution of 2,6-dibenzyloxy-3-bromo-pyridine (2.00 kg, 5.40 mol, 1.00 eq) in dioxane (12.0 L) was added 4,4,5,5-tetramethyl-1,3,2-dioxaborolane (760 g, 5.94 mol, 862 mL, 1.10 eq) and TEA (1.09 kg, 10.8 mol, 1.50 L, 2.00 eq), degassed and purged with N₂, then Pd(PPh₃)₂Cl₂ (189 g, 270 mmol, 0.05 eq) was added to the mixture. The mixture was stirred at 90° C. for 16 hrs. TLC (Petroleum ether/Ethyl acetate=10/1, Rr (starting material)=0.50, Rr (product)=0.55) showed starting material was consumed completely. The reaction mixture was filtered and concentrated. 4 reactions were combined. The residue was purified by column chromatography (SiO₂, Petroleum ether/Ethyl acetate=1/0 to 0/1). 2,6-bis(benzyloxy)-3-(4,4,5,5-tetramethyl-1,3,2-dioxaborolan-2-yl)pyridine (5.80 kg, 13.9 mol, 64.3% yield) was obtained. [M+H]⁺=418.3

### Step 2: 5-bromo-1,3-difluoro-2-iodobenzene

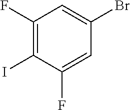

5 reactions were carried out in parallel.

To a solution of 1-bromo-3,5-difluoro-benzene (1.00 kg, 5.18 mol, 595 mL) in THF (4.00 L) was added LDA (2 M, 2.59 L) at −70° C. The mixture was stirred at −70° C. for 1 hr. Then to the mixture was added solution of I₂ (1.33 kg, 5.23 mol, 1.05 L) in THF (1.00 L) at −70° C. The mixture was stirred at −70° C. for 1 hr. TLC (Petroleum ether:Ethyl acetate=1:0, Rr (starting material)=0.86, R_{f} (product)=0.80) showed the starting material was consumed completely. The reaction mixture was poured into H₂O (5.00 L), extracted with EtOAc (3.00 L×2), 5 reactions were combined, the combined organic layers were washed by brine (5.00 L), dried over Na₂SO₄, filtered and concentrated in vacuum at 40° C. to give a residue. The crude product was triturated with petroleum ether (6.00 L). Compound 5-bromo-1,3-difluoro-2-iodo-benzene (4.50 kg, 14.1 mol, 54.4% yield) was obtained. [M+H]⁺=318.8

### Step 3: 2,6-bis(benzyloxy)-3-(4-bromo-2,6-difluorophenyl)pyridine

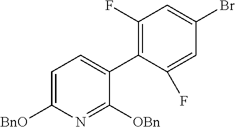 [SMILES: Fc1cc(Br)cc(F)c1-c1ccc(OCc2ccccc2)nc1OCc1ccccc1]

To a solution of 5-bromo-1,3-difluoro-2-iodo-benzene (4.50 kg, 14.1 mol) and 2,6-bis(benzyloxy)-3-(4,4,5,5-tetramethyl-1,3,2-dioxaborolan-2-yl)pyridine (5.89 kg, 14.1 mol) in dioxane (22.5 L) and H₂O (4.50 L) was added Pd(PPh₃)₄ (1.63 kg, 1.41 mol), K₃PO₄ (8.99 kg, 42.3 mol). The mixture was stirred at 90° C. for 12 hrs. TLC (Petroleum ether:Ethyl acetate=10:1, R_{f} (starting material)=0.84, R_{f} (product)=0.66) showed the starting material was consumed completely. The mixture was filtered and filtrate was extracted with EtOAc (5.00 L), the combined organic was washed with brine (5.00 L), dried over Na₂SO₄, concentrated in vacuum. The residue was purified by column chromatography (SiO₂, Petroleum ether:Ethyl acetate=1:0 to 1:1). Compound 2,6-dibenzyloxy-3-(4-bromo-2,6-difluoro-phenyl)pyridine (3.00 kg, 6.01 mol, 42.5% yield, 96.5% purity) was obtained. ¹H NMR (400 MHz, CHLOROFORM-d) 7.47-7.40 (m, 1H), 7.39-7.34 (m, 2H), 7.34-7.29 (m, 2H), 7.29-7.25 (m, 4H), 7.25-7.17 (m, 2H), 7.12-7.05 (m, 2H), 6.42 (d, J=8.0 Hz, 1H), 5.32 (s, 2H), 5.28 (s, 2H); [M+H]⁺=482.1.

### Intermediate 17: 1-(6-(2,6-dioxopiperidin-3-yl)-4-methylpyridin-3-yl)azetidine-3-carbaldehyde

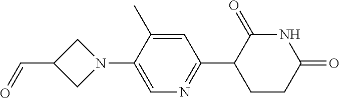 [SMILES: Cc1cc(C2CCC(=O)NC2=O)ncc1N1CC(C=O)C1]

The title compound was prepared in a procedure similar to that in intermediate 8 and 10. [M+H]⁺=288.3.

### Intermediate 18: 2′,6′-bis(benzyloxy)-3-fluoro-5-iodo-6-methyl-2,3′-bipyridine

### Step 1: 5-fluoro-2-methylpyridin-3-amine

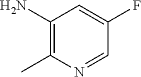

To a solution of 2-bromo-5-fluoropyridin-3-amine (20 g, 105.26 mmol) in dioxane/H₂O (200/40 mL) were added 2,4,6-trimethyl-1,3,5,2,4,6-trioxatriborinane (15.91 g, 126.32 mmol), Pd(dppf)Cl₂ (8.59 g, 10.53 mmol), and K₂CO₃ (43.57 g, 315.79 mmol). The resulting solution was stirred overnight at 120° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with MeOH/DCM (0˜10%) to afford 5-fluoro-2-methylpyridin-3-amine (12 g, 90.1%). [M+H]⁺=127.1.

### Step 2: 6-chloro-5-fluoro-2-methylpyridin-3-amine

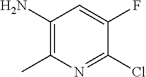

To a stirred mixture of 5-fluoro-2-methylpyridin-3-amine (6 g, 47.62 mmol) in DMF (120 mL) were added NCS (8.43 g, 47.62 mmol) at 0° C. The mixture was stirred overnight at 60° C. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with EA/PE (0˜50%) to afford 6-chloro-5-fluoro-2-methylpyridin-3-amine (3 g, 39.5%). [M+H]⁺=161.3.

### Step 3: 2′,6′-bis(benzyloxy)-3-fluoro-6-methyl-[2,3′-bipyridin]-5-amine

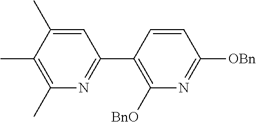 [SMILES: Cc1cc(-c2ccc(OCc3ccccc3)nc2OCc2ccccc2)nc(C)c1C]

To a solution of 6-chloro-5-fluoro-2-methylpyridin-3-amine (3 g, 18.63 mmol) in dioxane/H₂O (30/5 mL) were added 2,6-bis(benzyloxy)-3-(4,4,5,5-tetramethyl-1,3,2-dioxaborolan-2-yl)pyridine (9.32 g, 22.36 mmol), Pd(dppf)Cl₂ (1.52 g, 1.86 mmol), and K₂CO₃ (7.71 g, 55.89 mmol). The resulting solution was stirred overnight at 100° C. under N₂ atmosphere. After cooling to rt, diluted with H₂O and extracted with EtOAc. The combined organic layer was washed with brine, dried by Na₂SO₄ and concentrated. The filtrate was concentrated under reduced pressure. The crude product was purified by silica column chromatography (EA/PE=0-50%) to afford 2′,6′-bis(benzyloxy)-3-fluoro-6-methyl-[2,3′-bipyridin]-5-amine (5 g, 64.3%). [M+H]⁺=416.2.

### Step 4: 2′,6′-bis(benzyloxy)-3-fluoro-5-iodo-6-methyl-2,3′-bipyridine

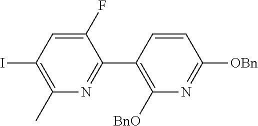 [SMILES: Cc1nc(-c2ccc(OCc3ccccc3)nc2OCc2ccccc2)c(F)cc1I]

To a solution of 2′,6′-bis(benzyloxy)-3-fluoro-6-methyl-[2,3′-bipyridin]-5-amine (3 g, 7.23 mmol) in ACN (30 mL) were added KI (6 g, 36.14 mmol), CuI (1.65 g, 8.67 mmol), and t-BuONO (3.72 g, 36.14 mmol). The resulting solution was stirred overnight at 80° C. under N₂ atmosphere. After cooling to rt, the mixture was diluted with H₂O and extracted with EtOAc. The combined organic layer was washed with brine, dried by Na₂SO₄ and concentrated. The filtrate was concentrated under reduced pressure. The crude product purified by silica column chromatography (EA/PE=0-15%) to afford 2′,6′-bis(benzyloxy)-3-fluoro-5-iodo-6-methyl-2,3′-bipyridine (2.5 g, 65.79%). [M+H]⁺=527.1.

### Intermediate 19: 3-(3-fluoro-5-((3R,4S)-3-fluoro-4-(piperazin-1-yl)piperidin-1-yl)-6-methylpyridin-2-yl)piperidine-2,6-dione

### Step 1: tert-butyl (3R,4S)-4-(4-benzylpiperazin-1-yl)-3-fluoropiperidine-1-carboxylate

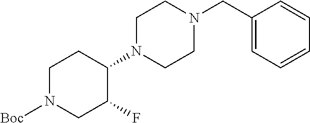 [SMILES: CC(C)(C)OC(=O)N1CC[C@H](N2CCN(Cc3ccccc3)CC2)[C@H](F)C1]

The solution of tert-butyl (3R,4S)-4-amino-3-fluoropiperidine-1-carboxylate (2.5 g, 11.5 mmol), N-benzyl-2-chloro-N-(2-chloroethyl)ethan-1-amine hydrogen chloride (3 g, 11.5 mmol) and NaHCO₃ (3.85 g, 45.8 mmol) in 50 mL EtOH was stirred at 80° C. for 16 hours. After LCMS showed the reaction was completed, the mixture was concentrated, diluted with DCM and washed with water. The organic layer was separated and concentrated. The mixture was purified by silica column chromatography (MeOH:DCM=0-4%) to afford tert-butyl (3R,4S)-4-(4-benzylpiperazin-1-yl)-3-fluoropiperidine-1-carboxylate (2 g, 5.3 mmol, 46.3%). [M+H]-378.6.

### Step 2: 1-benzyl-4-((3R,4S)-3-fluoropiperidin-4-yl)piperazine

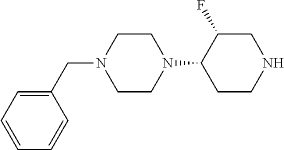 [SMILES: F[C@@H]1CNCC[C@@H]1N1CCN(Cc2ccccc2)CC1]

To a solution of tert-butyl (3R,4S)-4-(4-benzylpiperazin-1-yl)-3-fluoropiperidine-1-carboxylate (5 g, 13.2 mmol) in DCM (20 mL) was added TFA (10 mL). The reaction was stirred at room temperature for 2 hr and then concentrated in vacuo. The residue was dissolved in DCM (200 mL), washed with sat. NaHCO₃ solution (3×100 mL) and brine (50 mL), dried over Na₂SO₄, filtered and concentrated under reduced pressure to afford the product (3 g, 81.7%); [M+H]⁺=278.4.

### Step 3: 2′,6′-bis(benzyloxy)-5-((3R,4S)-4-(4-benzylpiperazin-1-yl)-3-fluoropiperidin-1-yl)-3-fluoro-6-methyl-2,3′-bipyridine

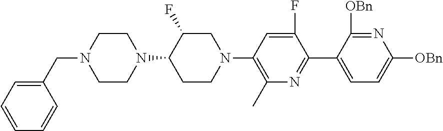 [SMILES: Cc1nc(-c2ccc(OCc3ccccc3)nc2OCc2ccccc2)c(F)cc1N1CC[C@H](N2CCN(Cc3ccccc3)CC2)[C@H](F)C1]

To a stirred solution of intermediate 18 (0.95 g, 1.8 mmol) in dioxane (20 mL) were added Cs₂CO₃ (1.2 g, 3.6 mmol), 1-benzyl-4-((3R,4S)-3-fluoropiperidin-4-yl)piperazine (0.5 g, 1.8 mmol) and Pd-Ruphos-G3 (0.3 g, 0.4 mmol) at room temperature under nitrogen atmosphere. The resulting mixture was stirred for 12 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with MeOH/DCM (0-5%) to afford the product (0.95 g, 78%). [M+H]⁺=676.7.

### Step 4: tert-butyl 4-((3R,4S)-1-(6-(2,6-dioxopiperidin-3-yl)-5-fluoro-2-methylpyridin-3-yl)-3-fluoropiperidin-4-yl)piperazine-1-carboxylate

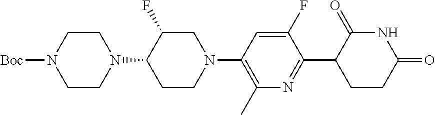 [SMILES: Cc1nc(C2CCC(=O)NC2=O)c(F)cc1N1CC[C@H](N2CCN(C(=O)OC(C)(C)C)CC2)[C@H](F)C1]

To a stirred mixture of 2′,6′-bis(benzyloxy)-5-((3R,4S)-4-(4-benzylpiperazin-1-yl)-3-fluoropiperidin-1-yl)-3-fluoro-6-methyl-2,3′-bipyridine (0.95 g, 1.4 mmol), di-tert-butyl dicarbonate (1.5 g, 7 mmol) in i-PrOH (40 mL) and DMF (40 mL) was added Pd/C (10 wt %, 1 g). The resulting mixture was stirred under hydrogen atmosphere (1 atm) at room temperature for 24 hr. Then the mixture was filtered and the filtrate was concentrated. The residue was purified by silica gel column chromatography, eluted with MeOH/DCM (0-7%) to afford the product (0.46 g, 64.5%). [M+H]⁺=508.4.

### Step 5: 3-(3-fluoro-5-((3R,4S)-3-fluoro-4-(piperazin-1-yl)piperidin-1-yl)-6-methylpyridin-2-yl)piperidine-2,6-dione

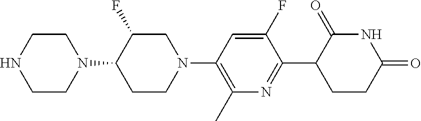 [SMILES: Cc1nc(C2CCC(=O)NC2=O)c(F)cc1N1CC[C@H](N2CCNCC2)[C@H](F)C1]

To a solution of tert-butyl 4-((3R,4S)-1-(6-(2,6-dioxopiperidin-3-yl)-5-fluoro-2-methylpyridin-3-yl)-3-fluoropiperidin-4-yl)piperazine-1-carboxylate (460 mg, 0.9 mmol) in DCM (10 mL) was added TFA (5 mL).

The reaction was stirred at room temperature for 2 hr and then concentrated in vacuo. The residue was dissolved in DCM (50 mL), washed with sat. NaHCO₃ solution (3×20 mL) and brine (20 mL), dried over Na₂SO₄, filtered and concentrated under reduced pressure to afford the product (260 mg, 70.4%). [M+H]⁺=408.5.

### Intermediate 20: 3-(5-((3R,4S)-3-fluoro-4-(piperazin-1-yl)piperidin-1-yl)-6-methylpyridin-2-yl)piperidine-2,6-dione

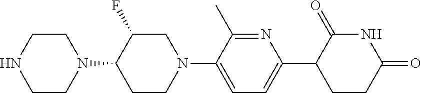 [SMILES: Cc1nc(C2CCC(=O)NC2=O)ccc1N1CC[C@H](N2CCNCC2)[C@H](F)C1]

The compound was prepared in a manner similar to that in Intermediate 19.

### Intermediate 21: 3-(4-methyl-5-(piperazin-1-yl)pyridin-2-yl)piperidine-2,6-dione

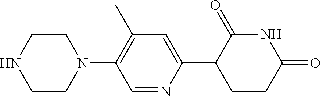 [SMILES: Cc1cc(C2CCC(=O)NC2=O)ncc1N1CCNCC1]

The compound was prepared in a manner similar to that in Intermediate 15.

### Intermediate 22: 3-(4-methyl-5-((S)-3-methylpiperazin-1-yl)pyridin-2-yl)piperidine-2,6-dione

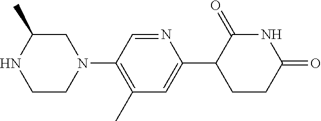 [SMILES: Cc1cc(C2CCC(=O)NC2=O)ncc1N1CCN[C@@H](C)C1]

The compound was prepared in a manner similar to that in Intermediate 15.

### Intermediate 23: 3-(5-((1S,4S)-2,5-diazabicyclo[2.2.1]heptan-2-yl)-6-methylpyridin-2-yl)piperidine-2,6-dione

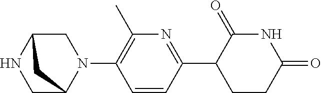 [SMILES: Cc1nc(C2CCC(=O)NC2=O)ccc1N1C[C@@H]2C[C@H]1CN2]

The compound was prepared in a manner similar to that in Intermediate 15.

### Intermediate 24: 3-(5-((R)-3,3-difluoro-4-(piperazin-1-yl)piperidin-1-yl)-6-methylpyridin-2-yl)piperidine-2,6-dione

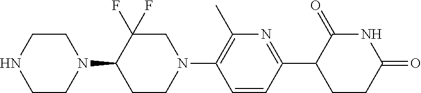 [SMILES: Cc1nc(C2CCC(=O)NC2=O)ccc1N1CC[C@@H](N2CCNCC2)C(F)(F)C1]

The compound was prepared in a manner similar to that in Intermediate 19.

### Intermediate 25: (7¹s,7³s,E)-1¹,2⁶-dimethyl-5-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

### Step 1: (7¹s,7³s,E)-1¹,2⁶-dimethyl-5⁶-(1,4-dioxa-8-azaspiro[4.5]decan-8-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

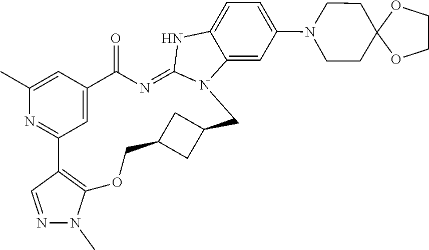 [SMILES: Cc1cc2cc(n1)-c1cnn(C)c1OC[C@H]1C[C@@H](CN3/C(=N/C2=O)Nc2ccc(N4CCC5(CC4)OCCO5)cc23)C1]

To the solution of intermediate 1 (3.0 g, 5.9 mmol), 1,4-dioxa-8-azaspiro[4.5]decane (1.7 g, 11.8 mmol) and t-BuONa (1.2 g, 11.8 mmol) in 80 mL DMA, were added Pd₂(dba)₃ (540 mg, 0.59 mmol) and RuPhos (550 mg, 1.18 mmol). The mixture was stirred at 100° C. for 3 hours under N₂. After LCMS showed the reaction was completed, the mixture was diluted with water and extracted by EtOAc. The organic layer was washed with brine, dried over anhydrous Na₂SO₄, filtered and concentrated under reduced pressure. The resulting mixture was purified by silica column chromatography (DCM:MeOH=100:1-15:1) to afford the product (2.5 g, 73.2%). [M+H]⁺=570.4.

### Step 2: (7¹s,7³s,E)-1¹,2⁶-dimethyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

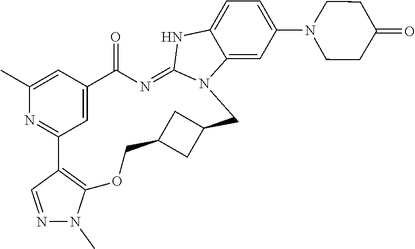 [SMILES: Cc1cc2cc(n1)-c1cnn(C)c1OC[C@H]1C[C@@H](CN3/C(=N/C2=O)Nc2ccc(N4CCC(=O)CC4)cc23)C1]

(7¹s,7³s,E)-1¹,2⁶-dimethyl-5⁶-(1,4-dioxa-8-azaspiro[4.5]decan-8-yl)-5²,5³-dihydro-1¹H,5′H-9-oxa-4-aza- 5(2,1)-benzo[d]imidazola-2(2,4)-pyridina-1(4,5)-pyrazola-7(1,3)-cyclobutanacyclononaphan-3-one (300 mg, 0.53 mmol) was placed in 25 mL round bottom flask with a magnetic stir bar. Then 4 mL concentrated HCl aqueous was added. The mixture was stirred at room temperature for 2 hours. To the mixture was added sat. aq. NaHCO₃ solution dropwise and the pH was adjusted to 6-7. The liquid was extracted with DCM. The combined organic phase was dried over anhydrous Na₂SO₄, concentrated in vacuum and purified with combiflash (DCM:MeOH=20:1) to afford the title compound (120 mg, 43.2% yield). [M+H]⁺=526.5.

### Intermediate 26: 2-(6-(2,6-dioxopiperidin-3-yl)-2,4-dimethylpyridin-3-yl)ethyl methanesulfonate

### Step 1: 2′,6′-bis(benzyloxy)-5-bromo-4,6-dimethyl-2,3′-bipyridine

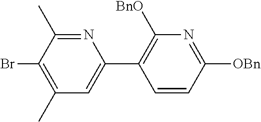 [SMILES: Cc1cc(-c2ccc(OCc3ccccc3)nc2OCc2ccccc2)nc(C)c1Br]

To a solution of 3-bromo-6-chloro-2,4-dimethylpyridine (9 g, 40.9 mmol) in dioxane/H₂O (100/20 mL) were added 2,6-bis(benzyloxy)-3-(4,4,5,5-tetramethyl-1,3,2-dioxaborolan-2-yl)pyridine (17.1 g, 40.9 mmol), Pd(PPh₃)₄ (4.64 g, 4.10 mmol), and K₂CO₃ (16.94 g, 122.73 mmol). The resulting solution was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-50%) to afford 2′,6′-bis(benzyloxy)-5-bromo-4,6-dimethyl-2,3′-bipyridine (12 g, 62.2%). [M+H]⁺=475.1.

### Step 2: 2′,6′-bis(benzyloxy)-4,6-dimethyl-5-vinyl-2,3′-bipyridine

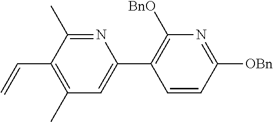 [SMILES: C=Cc1c(C)cc(-c2ccc(OCc3ccccc3)nc2OCc2ccccc2)nc1C]

To a stirred mixture of 2′,6′-bis(benzyloxy)-5-bromo-4,6-dimethyl-2,3′-bipyridine (3 g, 6.31 mmol), 4,4,5,5-tetramethyl-2-vinyl-1,3,2-dioxaborolane (1.17 g, 7.58 mmol), and K₂CO₃ (2.61 g, 18.93 mmol) in 1,4-dioxane (30 mL) and H₂O (6 mL) was added Pd(dpppf)Cl₂ (514.1 mg, 0.63 mmol). The resulting solution was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-20%) to afford 2′,6′-bis(benzyloxy)-4,6-dimethyl-5-vinyl-2,3′-bipyridine (1.7 g, 63.9%). [M+H]⁺=423.0.

### Step 3: 2-(2′,6′-bis(benzyloxy)-4,6-dimethyl-[2,3′-bipyridin]-5-yl)ethan-1-ol

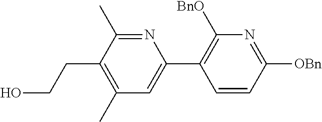 [SMILES: Cc1cc(-c2ccc(OCc3ccccc3)nc2OCc2ccccc2)nc(C)c1CCO]

To a stirred mixture of 2′,6′-bis(benzyloxy)-4,6-dimethyl-5-vinyl-2,3′-bipyridine (1.7 g, 4.01 mmol) in THF (20 mL) was added 9-BBN (0.5 M in THF, 40 mL, 20.0 mmol) dropwise at 0° C. The reaction mixture was stirred overnight at rt, then NaOH (2M in water, 4 mL, 8.03 mmol) and H₂O₂ (30%, 40.2 mL, 12.1 mmol) were added at 0° C. The reaction mixture was stirred at rt for 2 h, The mixture was diluted with H₂O and extracted with EA, and the residue was purified by column chromatography (EA/PE=0-35%) to afford 2-(2′,6′-bis(benzyloxy)-4,6-dimethyl-[2,3′-bipyridin]-5-yl)ethan-1-ol (1.4 g, 79.5%). [M+H]⁺=441.0.

### Step 4: 3-(5-(2-hydroxyethyl)-4,6-dimethylpyridin-2-yl)piperidine-2,6-dione

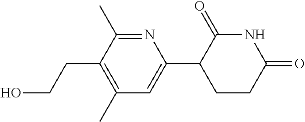 [SMILES: Cc1cc(C2CCC(=O)NC2=O)nc(C)c1CCO]

To a stirred solution 2-(2′,6′-bis(benzyloxy)-4,6-dimethyl-[2,3′-bipyridin]-5-yl)ethan-1-ol (1.4 g, 8.31 mmol) in THE (50 mL) was added Pd/C (10 wt %, 1.5 g). The resulting mixture was degassed under reduced pressure and purged with H₂ for five times, then stirred overnight at 50° C. The resulting mixture was filtered, the filter cake was washed with THE. The filtrate was concentrated under reduced pressure. And the residue was purified by column chromatography (EA/PE=30-50%) to afford 3-(5-(2-hydroxyethyl)-4,6-dimethylpyridin-2-yl)piperidine-2,6-dione (855.7 mg, 83.0%). [M+H]⁺=263.1. ¹H NMR (300 MHz, DMSO) δ 10.80 (s, 1H), 6.96 (s, 1H), 4.83-4.74 (m, 1H), 3.83 (m, 1H), 3.52 (m, 2H), 2.78 (t, J=9 Hz, 2H), 2.64-2.54 (m, 3H), 2.44 (s, 3H), 2.29 (s, 3H), 2.26-2.02 (m, 1H).

### Step 5: 2-(6-(2,6-dioxopiperidin-3-yl)-2,4-dimethylpyridin-3-yl)ethyl methanesulfonate

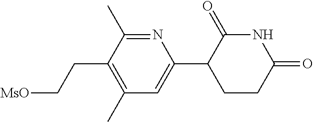 [SMILES: Cc1cc(C2CCC(=O)NC2=O)nc(C)c1CCOS(C)(=O)=O]

To a solution of 3-(5-(2-hydroxyethyl)-4,6-dimethylpyridin-2-yl)piperidine-2,6-dione (300 mg, 1.15 mmol) and Et₃N (347 mg, 3.44 mmol) in DCM (6 mL) and THE (6 mL), MsCl (172 mg, 1.49 mmol) was slowly added at 0° C. The mixture was stirred at 25° C. for 2 hours. After LCMS shown the reaction was completed. The mixture was quenched with water (10 mL). The organic phase was separated and concentrated in vacuum. The residue was purified by prep-TLC (DCM:MeOH=20:1) to afford product (220 mg, 56.5% yield). [M+H]⁺=341.5.

### Intermediate 27: 2-(6-2,6-dioxopiperidin-3-yl)-2-methylpyridin-3-yl)ethyl methanesulfonate

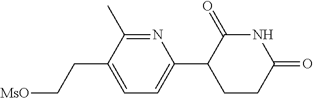 [SMILES: Cc1nc(C2CCC(=O)NC2=O)ccc1CCOS(C)(=O)=O]

The compound was prepared in a manner similar to that in Intermediate 26.

### Intermediate 28: 3-(5-((3S,4R)-3-fluoro-4-(piperazin-1-yl)piperidin-1-yl)-6-methylpyridin-2-yl)piperidine-2,6-dione

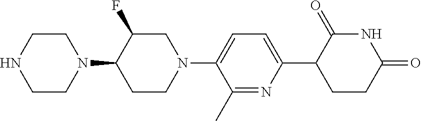 [SMILES: Cc1nc(C2CCC(=O)NC2=O)ccc1N1CC[C@@H](N2CCNCC2)[C@@H](F)C1]

The compound was prepared in a manner similar to that in Intermediate 19

### Intermediate 29: 3-(5-((3R,4R)-3-fluoro-4-(piperazin-1-yl)piperidin-1-yl)-6-methylpyridin-2-yl)piperidine-2,6-dione

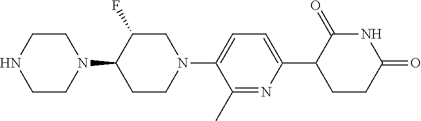 [SMILES: Cc1nc(C2CCC(=O)NC2=O)ccc1N1CC[C@@H](N2CCNCC2)[C@H](F)C1]

The compound was prepared in a manner similar to that in Intermediate 19.

### Intermediate 30: 3-(6-methyl-5-((S)-3-methylpiperazin-1-yl)pyridin-2-yl)piperidine-2,6-dione

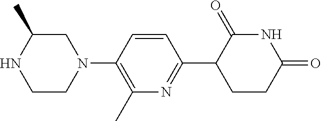 [SMILES: Cc1nc(C2CCC(=O)NC2=O)ccc1N1CCN[C@@H](C)C1]

The compound was prepared in a manner similar to that in Intermediate 15.

### Intermediate 31: 3-(5-((S)-3,3-difluoro-4-(piperazin-1-yl)piperidin-1-yl)-6-methylpyridin-2-yl)piperidine-2,6-dione

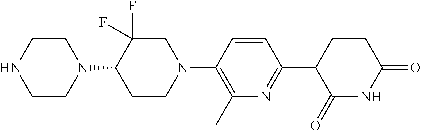 [SMILES: Cc1nc(C2CCC(=O)NC2=O)ccc1N1CC[C@H](N2CCNCC2)C(F)(F)C1]

The compound was prepared in a manner similar to that in Intermediate 19.

### Intermediate 32: 3-(5-((3S,4R)-3-fluoro-4-(piperazin-1-yl)piperidin-1-yl)-4-methylpyridin-2-yl)piperidine-2,6-dione

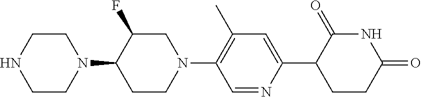 [SMILES: Cc1cc(C2CCC(=O)NC2=O)ncc1N1CC[C@@H](N2CCNCC2)[C@@H](F)C1]

The compound was prepared in a manner similar to that in Intermediate 19.

### Intermediate 33: 3-(5-((3R,4S)-3-fluoro-4-(piperazin-1-yl)piperidin-1-yl)-4-methylpyridin-2-yl)piperidine-2,6-dione

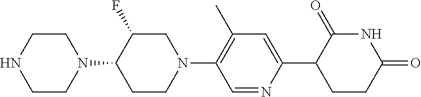 [SMILES: Cc1cc(C2CCC(=O)NC2=O)ncc1N1CC[C@H](N2CCNCC2)[C@H](F)C1]

The compound was prepared in a manner similar to that in Intermediate 19.

### Intermediate 34: 3-(2,6-difluoro-4-((3S,4R)-3-fluoro-4-(piperazin-1-yl)piperidin-1-yl)phenyl)piperidine-2,6-dione

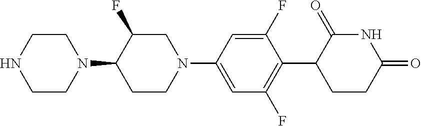 [SMILES: O=C1CCC(c2c(F)cc(N3CC[C@@H](N4CCNCC4)[C@@H](F)C3)cc2F)C(=O)N1]

The compound was prepared in a manner similar to that in Intermediate 19.

### Intermediate 35: 3-(5-((3R,4R)-3-fluoro-4-(piperazin-1-yl)piperidin-1-yl)-4-methylpyridin-2-yl)piperidine-2,6-dione

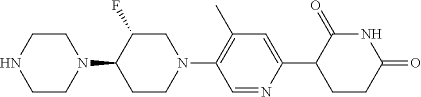 [SMILES: Cc1cc(C2CCC(=O)NC2=O)ncc1N1CC[C@@H](N2CCNCC2)[C@H](F)C1]

The compound was prepared in a manner similar to that in Intermediate 19.

### Intermediate 36: 3-(6-ethyl-5-(4-oxopiperidin-1-yl)pyridin-2-yl)piperidine-2,6-dione

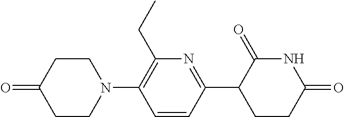 [SMILES: CCc1nc(C2CCC(=O)NC2=O)ccc1N1CCC(=O)CC1]

The compound was prepared in a manner similar to that in Intermediate 10.

### Intermediate 37: 3-(4,6-dimethyl-5-(4-oxopiperidin-1-yl)pyridin-2-yl)piperidine-2,6-dione

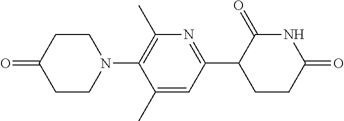 [SMILES: Cc1cc(C2CCC(=O)NC2=O)nc(C)c1N1CCC(=O)CC1]

The compound was prepared in a manner similar to that in Intermediate 10.

### Intermediate 38: 3-(3-fluoro-6-methyl-5-(4-oxopiperidin-1-yl)pyridin-2-yl)piperidine-2,6-dione

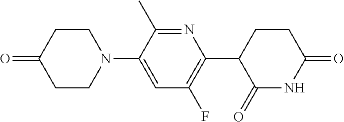 [SMILES: Cc1nc(C2CCC(=O)NC2=O)c(F)cc1N1CCC(=O)CC1]

The compound was prepared in a manner similar to that in Intermediate 10.

### Intermediate 39: (7¹s,7³s,E)-5⁶-(4-(azetidin-3-yl)piperazin-1-yl)-1¹,2⁶-dimethyl-5²,5³-dihydro-1H,5H-9-oxa-4-aza-5(2,1)-benzo[d]imidazola-2(2,4)-pyridina-1(4,5)-pyrazola-7(1,3)-cyclobutanacyclononaphan-3-one

### Step 1: tert-butyl 3-(4-((7¹s,7³s,E)-1¹,2⁶-dimethyl-3-oxo-5²,5³-dihydro-1¹H,5¹H-9-oxa-4-aza-5(2,1)- benzo[d]imidazola-2(2,4)-pyridina-1(4,5)-pyrazola-7(1,3)-cyclobutanacyclononaphane-5′-yl)piperazin-1-yl)azetidine-1-carboxylate

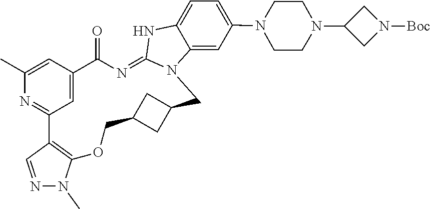 [SMILES: Cc1cc2cc(n1)-c1cnn(C)c1OC[C@H]1C[C@@H](CN3/C(=N/C2=O)Nc2ccc(N4CCN(C5CN(C(=O)OC(C)(C)C)C5)CC4)cc23)C1]

To the solution of (7¹s,7³s,E)-5⁶-bromo-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (507.0 mg, 1.0 mmol), tert-butyl 3-(piperazin-1-yl)azetidine-1-carboxylate (313.0 mg, 1.3 mmol) and t-BuONa (288 mg, 3.0 mmol) in 10 mL DMA, were added Pd₂(dba)₃ (183 mg, 0.2 mmol) and RuPhos (186 mg, 0.4 mmol). The mixture was stirred at 90° C. for 1.5 hours under N₂. The mixture was diluted with aqueous NH₄Cl (20 mL) and extracted by DCM (25 mL×2). The organic layer was washed with brine, dried over anhydrous Na₂SO₄, filtered and concentrated under reduced pressure. The resulting mixture was purified by silica column chromatography (DCM:MeOH=100:1-90:10) to afford the product (450 mg, 67.5%). [M+H]⁺=668.7.

### Step 2: (7¹s,7³s,E)-5⁶-(4-(azetidin-3-yl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

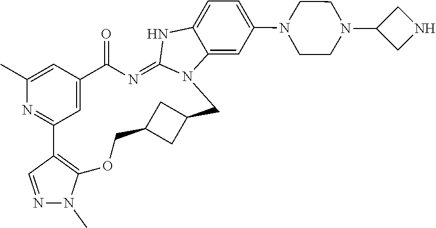 [SMILES: Cc1cc2cc(n1)-c1cnn(C)c1OC[C@H]1C[C@@H](CN3/C(=N/C2=O)Nc2ccc(N4CCN(C5CNC5)CC4)cc23)C1]

To a solution of tert-butyl 3-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)azetidine-1-carboxylate (400 mg, 0.599 mmol) in DCM (6 mL) was added TFA (2 mL). The reaction solution was stirred at r.t for 1.5 hrs and then concentrated in vacuo. The residue was dissolved in DCM and then washed with sat NaHCO₃ solution (5 mL) and brine (5 mL), dried over Na₂SO₄, filtered and concentrated in vacuum to afford the product (290 mg, 85.2%). [M+H]⁺=568.7.

### Intermediate 40: 3-(5-(4-((R)-3,3-difluoropiperidin-4-yl)piperazin-1-yl)-6-methylpyridin-2-yl)piperidine-2,6-dione

### Step 1: tert-butyl (S)-4-(4-benzylpiperazin-1-yl)-3,3-difluoropiperidine-1-carboxylate

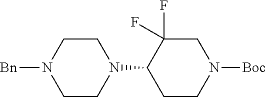 [SMILES: CC(C)(C)OC(=O)N1CC[C@H](N2CCN(Cc3ccccc3)CC2)C(F)(F)C1]

The solution of tert-butyl (S)-4-amino-3,3-difluoropiperidine-1-carboxylate (2.5 g, 10.6 mmol), N-benzyl-2-chloro-N-(2-chloroethyl)ethan-1-amine hydrochloride (2.84 g, 11.6 mmol) and NaHCO₃ (3.56 g, 42.4 mmol) in 50 mL EtOH was stirred at 80° C. for 16 hours. After LCMS shown the reaction was completed, the mixture was concentrated, diluted with water and extracted by DCM. The organic layer was separated and concentrated. The mixture was purified by silica column chromatography (MeOH:DCM=0-4%) to afford tert-butyl (S)-4-(4-benzylpiperazin-1-yl)-3,3-difluoropiperidine-1-carboxylate (2 g, 5.0 mmol, 47.2%). [M+H]⁺=396.3.

### Step 2: tert-butyl (S)-3,3-difluoro-4-(piperazin-1-yl)piperidine-1-carboxylate

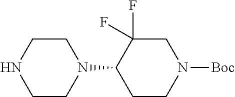 [SMILES: CC(C)(C)OC(=O)N1CC[C@H](N2CCNCC2)C(F)(F)C1]

To a solution of tert-butyl (S)-4-(4-benzylpiperazin-1-yl)-3,3-difluoropiperidine-1-carboxylate (2 g, 5.06 mmol) in MeOH (30 mL) was added Pd/C (20 wt %, 400 mg). The reaction was stirred at room temperature for 2 h. The reaction was filtrated and the filtrate was concentrated to afford the product tert-butyl (S)-3,3-difluoro-4-(piperazin-1-yl)piperidine-1-carboxylate (1.2 g, 77.5%). [M+H]⁺306.4.

### Step 3: tert-butyl (S)-4-(4-(2′,6′-bis(benzyloxy)-6-methyl-[2,3′-bipyridin]-5-yl)piperazin-1-yl)-3,3-difluoropiperidine-1-carboxylate

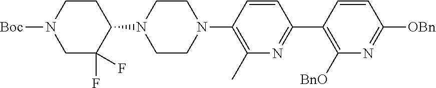 [SMILES: Cc1nc(-c2ccc(OCc3ccccc3)nc2OCc2ccccc2)ccc1N1CCN([C@H]2CCN(C(=O)OC(C)(C)C)CC2(F)F)CC1]

To a solution of 2′,6′-bis(benzyloxy)-5-bromo-6-methyl-2,3′-bipyridine 4 (lg, 2.17 mmol) in dioxane (20 mL) were added Cs₂CO₃ (1.4 g, 4.34 mmol), tert-butyl (S)-3,3-difluoro-4-(piperazin-1-yl)piperidine-1-carboxylate (997 mg, 3.26 mmol), Pd₂(dba)₃ (393 mg, 0.43 mmol) and RuPhos (400 mg, 0.86 mmol). The resulting mixture was stirred for 16 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with MeOH/DCM (0-5%) to afford the product tert-butyl (S)-4-(4-(2′,6′-bis(benzyloxy)-6-methyl-[2,3′-bipyridin]-5-yl)piperazin-1-yl)-3,3-difluoropiperidine-1-carboxylate (800 mg, 53.7%). [M+H]⁺=686.7.

### Step 4: tert-butyl (4S)-4-(4-(6-(2,6-dioxopiperidin-3-yl)-2-methylpyridin-3-yl)piperazin-1-yl)-3,3-difluoropiperidine-1-carboxylate

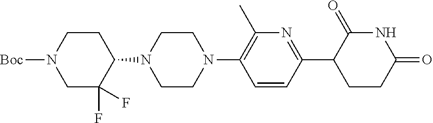 [SMILES: Cc1nc(C2CCC(=O)NC2=O)ccc1N1CCN([C@H]2CCN(C(=O)OC(C)(C)C)CC2(F)F)CC1]

To a stirred mixture of tert-butyl (S)-4-(4-(2′,6′-bis(benzyloxy)-6-methyl-[2,3′-bipyridin]-5-yl)piperazin-1-yl)-3,3-difluoropiperidine-1-carboxylate (800 mg, 1.17 mmol) in i-PrOH (10 mL) and DMF (10 mL) was added Pd/C (20 wt %, 160 mg). The resulting mixture was stirred under hydrogen atmosphere (1 atm) at 50° C. for 24 hr. Then the mixture was filtered and the filtrate was concentrated. The residue was purified by silica gel column chromatography, eluted with DCM/MeOH (0-7%) to afford the product (400 mg, 67.4%). [M+H]⁺=508.6.

### Step 5: 3-(5-(4-((S)-3,3-difluoropiperidin-4-yl)piperazin-1-yl)-6-methylpyridin-2-yl)piperidine-2,6-dione

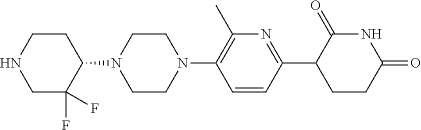 [SMILES: Cc1nc(C2CCC(=O)NC2=O)ccc1N1CCN([C@H]2CCNCC2(F)F)CC1]

To a solution of tert-butyl (4S)-4-(4-(6-(2,6-dioxopiperidin-3-yl)-2-methylpyridin-3-yl)piperazin-1-yl)-3,3-difluoropiperidine-1-carboxylate (400 mg, 0.79 mmol) in DCM (10 mL) was added HCl (4M in dioxane) (5 mL). The reaction was stirred at room temperature for 2 hr and then concentrated in vacuo to afford the product (260 mg, 80.8%). [M+H]⁺=408.5.

Example 1: 3-(5-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)-4-methylpyridin-2-yl)piperidine-2,6-dione

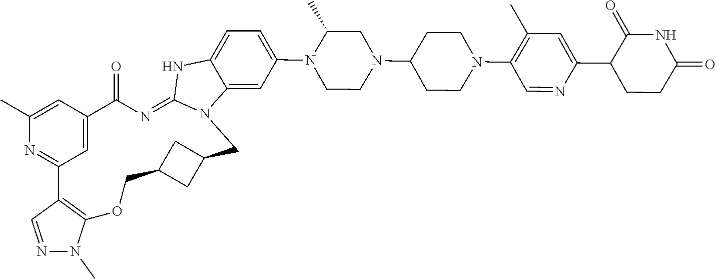 [SMILES: Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)C[C@H]4C)cc23)C1]

The titled compound was prepared in a manner similar to that in Example 33.

¹H NMR (500 MHz, DMSO) δ 12.34 (s, 1H), 10.73 (s, 1H), 8.59 (s, 1H), 8.08 (s, 1H), 7.82 (s, 1H), 7.39 (s, 1H), 7.29 (d, J=8.7 Hz, 1H), 7.07 (s, 1H), 6.94 (s, 1H), 6.82 (d, J=8.7 Hz, 1H), 3.89-3.81 (m, 3H), 3.65 (s, 3H), 3.15-3.11 (m, 3H), 3.01-2.90 (m, 2H), 2.88-2.82 (m, 4H), 2.70-2.63 (m, 5H), 2.59-2.54 (m, 4H), 2.49 (s, 5H), 2.31 (d, J=12.6 Hz, 2H), 2.19 (s, 3H), 2.16-2.09 (m, 1H), 2.07-2.00 (m, 1H), 1.84 (s, 2H), 1.64-1.45 (m, 2H), 0.91 (d, J=6.1 Hz, 3H). [M+H]⁺=812.7.

### Example 2: (R)-3-(4-((S)-4-(4-(((7¹r,7³S,E)-1¹,2⁶-dimethyl-3-oxo-5²,5³-dihydro-1²H,5′H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carbonyl)-3,3-dimethylpyrrolidin-1-yl)-2,6-difluorophenyl)piperidine-2,6-dione

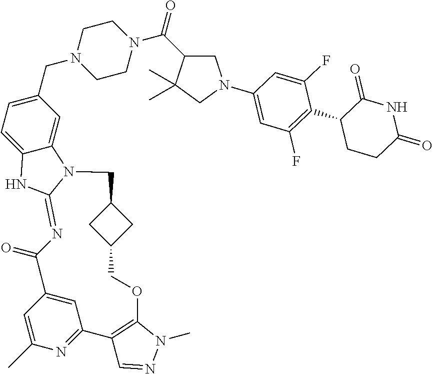 [SMILES: Cc1cc2cc(n1)-c1cnn(C)c1OC[C@H]1C[C@H](CN3/C(=N/C2=O)Nc2ccc(CN4CCN(C(=O)C5CN(c6cc(F)c([C@H]7CCC(=O)NC7=O)c(F)c6)CC5(C)C)CC4)cc23)C1]

The titled compound was prepared in a manner similar to that in Example 3 with intermediate 4.

¹H NMR (500 MHz, DMSO) δ 12.61 (s, 1H), 10.83 (s, 1H), 8.46 (s, 1H), 7.87 (s, 1H), 7.49-7.43 (m, 3H), 7.23-7.21 (m, 1H), 6.23-6.20 (m, 2H), 4.77-4.68 (m, 1H), 4.59-4.46 (m, 1H), 4.30-4.24 (m, 1H), 4.18-4.12 (m, 1H), 4.04-3.98 (m, 1H), 3.73 (s, 3H), 3.68-3.64 (m, 1H), 3.60-3.56 (m, 4H), 3.53-3.48 (m, 3H), 3.47-3.40 (m, 2H), 3.25-3.21 (m, 1H), 2.86-2.60 (m, 2H), 2.54 (s, 4H), 2.45-2.41 (m, 2H), 2.40-2.35 (m, 2H), 2.20-1.99 (m, 4H), 1.98-1.62 (m, 3H), 1.17 (s, 3H), 0.98 (s, 3H); [M+H]⁺=875.8.

### Example 3: (R)-3-(4-(3-(4-(((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carbonyl)azetidin-1-yl)-2,6-difluorophenyl)piperidine-2,6-dione

### Step 1: Tert-butyl 4-(3-fluoro-4-nitrobenzyl)piperazine-1-carboxylate

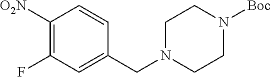 [SMILES: CC(C)(C)OC(=O)N1CCN(Cc2ccc([N+](=O)[O-])c(F)c2)CC1]

To a solution of 3-fluoro-4-nitrobenzaldehyde (5 g, 29.6 mmol) and tert-butyl piperazine-1-carboxylate (6.6 g, 35.5 mmol) in DCE (100 mL) was added STAB (12.55 g, 59.2 mmol). The reaction mixture was stirred at r.t for 6 hrs. The reaction was quenched with sat. aq. NaHCO₃ (60 mL), and the resulting mixture was extracted with DCM (100 mL×3). The combined organic phase was washed with brine (130 mL), dried over Na₂SO₄, filtered and concentrated in vacuum. The residue was purified by silica gel column (PE:EA=1:1) to afford tert-butyl 4-(3-fluoro-4-nitrobenzyl)piperazine-1-carboxylate (8.6 g, 86%). [M+H]⁺=340.2.

### Step 2: Tert-butyl 4-(3-((((1s,3s)-3-(methoxycarbonyl)cyclobutyl)methyl)amino)-4-nitrobenzyl)piperazine-1-carboxylate

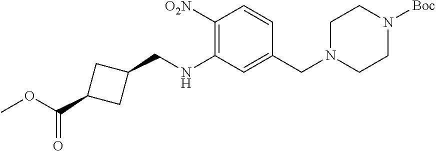 [SMILES: COC(=O)[C@H]1C[C@@H](CNc2cc(CN3CCN(C(=O)OC(C)(C)C)CC3)ccc2[N+](=O)[O-])C1]

To a mixture of tert-butyl 4-(3-fluoro-4-nitrobenzyl)piperazine-1-carboxylate (4 g, 11.8 mmol) and methyl (1s,3s)-3-(aminomethyl)cyclobutane-1-carboxylate hydrochloride (2.0 g, 14.1 mmol) in CH₃CN (80 mL) was added DIEA (6.1 g, 47.0 mmol). The reaction was stirred at 70° C. for 16 hrs. After cooling to rt, the reaction was concentrated in vacuum. The residue was purified by silica gel column (PE:EA=1:2) to afford tert-butyl 4-(3-((((1s,3s)-3-(methoxycarbonyl)cyclobutyl)methyl)amino)-4-nitrobenzyl)piperazine-1-carboxylate (4.3 g, 79.6%). [M+H]⁺463.5.

### Step 3: Tert-butyl 4-(3-((((1s,3s)-3-(hydroxymethyl)cyclobutyl)methyl)amino)-4-nitrobenzyl)piperazine-1-carboxylate

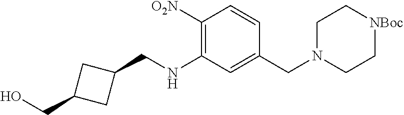 [SMILES: CC(C)(C)OC(=O)N1CCN(Cc2ccc([N+](=O)[O-])c(NC[C@H]3C[C@@H](CO)C3)c2)CC1]

A solution of tert-butyl 4-(3-((((1s,3s)-3-(methoxycarbonyl)cyclobutyl)methyl)amino)-4-nitrobenzyl)piperazine-1-carboxylate (2.0 g, 4.3 mmol) in THF (50 mL) was cooled to 0° C., and then LiAlH₄ (246 mg, 6.5 mmol) was added. The mixture was stirred at 0° C. for 1 hr. The reaction was quenched with sat. aq. NH₄Cl (50 mL), and the resulting mixture was extracted with DCM (40 mL×3). The combined organic phase was washed with brine (50 mL), dried over Na₂SO₄, filtered and concentrated in vacuum. The residue was purified by silica gel column (PE:EA=1:2) to afford tert-butyl 4-(3-((((1s,3s)-3-(hydroxymethyl)cyclobutyl)methyl)amino)-4-nitrobenzyl)piperazine-1-carboxylate (1.6 g, 84.2%). [M+H]⁺=435.4.

### Step 4: Tert-butyl 4-(3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)-4-nitrobenzyl)piperazine-1-carboxylate

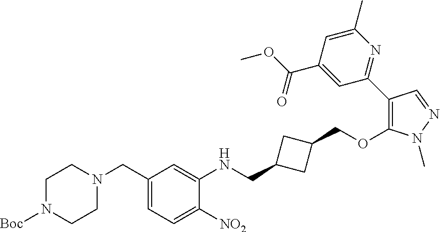 [SMILES: COC(=O)c1cc(C)nc(-c2cnn(C)c2OC[C@H]2C[C@@H](CNc3cc(CN4CCN(C(=O)OC(C)(C)C)CC4)ccc3[N+](=O)[O-])C2)c1]

To a solution of tert-butyl 4-(3-((((1s,3s)-3-(hydroxymethyl)cyclobutyl)methyl)amino)-4-nitrobenzyl)piperazine-1-carboxylate (499 mg, 1.15 mmol) and methyl 2-(5-hydroxy-1-methyl-1H-pyrazol-4-yl)-6-methylisonicotinate (312 mg, 1.26 mmol) in THF (10 mL) was added PPh₃ (453 mg, 1.73 mmol) and DIAD (349 mg, 1.73 mmol). The reaction was stirred at rt for 2 hrs. The reaction was concentrated in vacuum, the residue was purified by silica gel column (DCM:CH₃OH=20:1) to afford tert-butyl 4-(3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)-4-nitrobenzyl)piperazine-1-carboxylate (550 mg, 72.1%). [M+H]⁺=664.6.

### Step 5: Tert-butyl 4-(4-amino-3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)benzyl)piperazine-1-carboxylate

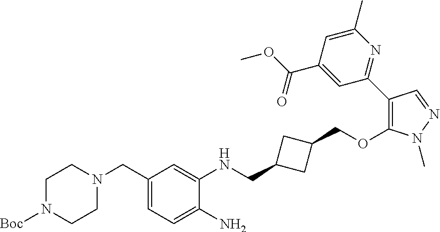 [SMILES: COC(=O)c1cc(C)nc(-c2cnn(C)c2OC[C@H]2C[C@@H](CNc3cc(CN4CCN(C(=O)OC(C)(C)C)CC4)ccc3N)C2)c1]

To a solution of tert-butyl 4-(3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)-4-nitrobenzyl)piperazine-1-carboxylate (550 mg, 0.83 mmol) in THF (15 mL) was added Raney-Ni (220 mg). The reaction was stirred at rt for 2 hrs under H₂ atmosphere. The catalyst was filtered off and the filtrate was concentrated in vacuum to afford tert-butyl 4-(4-amino-3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)benzyl)piperazine-1-carboxylate (500 mg, 95.2%). [M+H]⁺=634.6.

### Step 6: Tert-butyl 4-((2-imino-3-(((1s,3s)-3-(((4-(4-(methoxycarbonyl)-6-methylpyridin-2-yl)-1-methyl-1H-pyrazol-5-yl)oxy)methyl)cyclobutyl)methyl)-2,3-dihydro-1H-benzo[d]imidazol-5-yl)methyl)piperazine-1-carboxylate

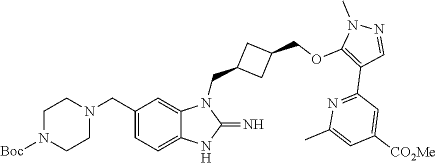 [SMILES: COC(=O)c1cc(C)nc(-c2cnn(C)c2OC[C@H]2C[C@@H](Cn3c(=N)[nH]c4ccc(CN5CCN(C(=O)OC(C)(C)C)CC5)cc43)C2)c1]

To a solution of tert-butyl 4-(4-amino-3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)benzyl)piperazine-1-carboxylate (500 mg, 0.79 mmol) in DCM (5 mL) and CH₃OH (2 mL) was added BrCN (166 mg, 1.58 mmol). The reaction was stirred at r.t for 16 hrs. The reaction was concentrated in vacuo, and the residue was purified by silica gel column (DCM:MeOH=8:1) to afford tert-butyl 4-((2-imino-3-(((1s,3s)-3-(((4-(4-(methoxycarbonyl)-6-methylpyridin-2-yl)-1-methyl-1H-pyrazol-5-yl)oxy)methyl)cyclobutyl)methyl)-2,3-dihydro-1H-benzo[d]imidazol-5-yl)methyl)piperazine-1-carboxylate (490 mg, 94%). [M+H]⁺=659.5.

### Step 7: 2-(5-(((1s,3s)-3-((6-((4-(tert-butoxycarbonyl)piperazin-1-yl)methyl)-2-imino-2,3-dihydro-1H-benzo[d]imidazol-1-yl)methyl)cyclobutyl)methoxy)-1-methyl-1H-pyrazol-4-yl)-6-methylisonicotinic acid

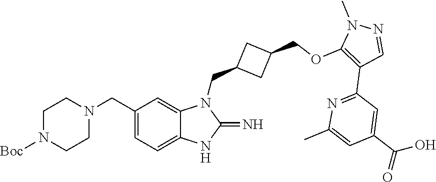 [SMILES: Cc1cc(C(=O)O)cc(-c2cnn(C)c2OC[C@H]2C[C@@H](Cn3c(=N)[nH]c4ccc(CN5CCN(C(=O)OC(C)(C)C)CC5)cc43)C2)n1]

To a solution of tert-butyl 4-((2-imino-3-(((1s,3s)-3-(((4-(4-(methoxycarbonyl)-6-methylpyridin-2-yl)-1-methyl-1H-pyrazol-5-yl)oxy)methyl)cyclobutyl)methyl)-2,3-dihydro-1H-benzo[d]imidazol-5-yl)methyl)piperazine-1-carboxylate (490 mg, 0.74 mmol) in THE (15 mL) and H₂O (5 m1) was added LiOH monohydrate (156 mg, 3.72 mmol). The reaction was stirred at rt for 5 hrs. The pH of reaction mixture was adjusted to 5-6 with 1N HCl solution, and then extracted with DCM (20 mL×3). The combined organic phase was washed with brine (30 mL), dried over Na₂SO₄, filtered and concentrated in vacuum to afford 2-(5-(((1s,3s)-3-((6-((4-(tert-butoxycarbonyl)piperazin-1-yl)methyl)-2-imino-2,3-dihydro-1H-benzo[d]imidazol-1-yl)methyl)cyclobutyl)methoxy)-1-methyl-1H-pyrazol-4-yl)-6-methylisonicotinic acid (450 mg, 94.3%). [M+H]⁺=645.6.

### Step 8: Tert-butyl 4-(((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carboxylate

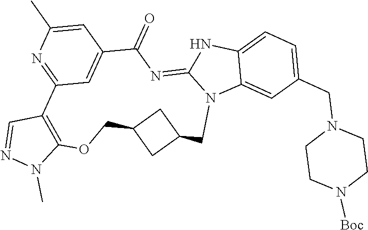 [SMILES: Cc1cc2cc(n1)-c1cnn(C)c1OC[C@H]1C[C@@H](CN3/C(=N/C2=O)Nc2ccc(CN4CCN(C(=O)OC(C)(C)C)CC4)cc23)C1]

To a solution of 2-(5-(((1s,3s)-3-((6-((4-(tert-butoxycarbonyl)piperazin-1-yl)methyl)-2-imino-2,3-dihydro-1H-benzo[d]imidazol-1-yl)methyl)cyclobutyl)methoxy)-1-methyl-1H-pyrazol-4-yl)-6-methylisonicotinic acid (450 mg, 0.7 mmol) in DMF (10 mL) was added HATU (265 mg, 0.7 mmol) and DIEA (181 mg, 1.4 mmol). The reaction was stirred at r.t for 30 min. The reaction was quenched with water (10 mL), and the resulting mixture was extracted with EA (15 mL×3). The combined organic phase was washed with brine (10 mL×3), dried over Na₂SO₄, filtered and concentrated in vacuum. The residue was purified by silica gel column (DCM:CH₃OH=15:1) to afford tert-butyl 4-(((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carboxylate (220 mg, 50.1%). [M+H]⁺=627.5.

### Step 9: (7¹s,7³s,E)-1¹,2⁶-dimethyl-5⁶-(piperazin-1-ylmethyl)-5²,5³-dihydro-1¹H,5¹H-9-oxa-4-aza-5(2,1)- benzo[d]imidazola-2(2,4)-pyridina-1(4,5)-pyrazola-7(1,3)-cyclobutanacyclononaphan-3-one

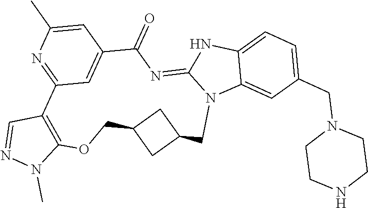 [SMILES: Cc1cc2cc(n1)-c1cnn(C)c1OC[C@H]1C[C@@H](CN3/C(=N/C2=O)Nc2ccc(CN4CCNCC4)cc23)C1]

To a solution of tert-butyl 4-(((7¹s,7³s,E)-1¹,2⁶-dimethyl-3-oxo-5²,5³-dihydro-1¹H,5′H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carboxylate (220 mg, 0.35 mmol) in DCM (8 mL) was added TFA (2 mL). The reaction was stirred at r.t for 1 hrs. The reaction was concentrated in vacuum. The residue was dissolved in DCM (15 mL), washed with sat. aq. NaHCO₃ solution (10 mL) and brine (10 mL), dried over Na₂SO₄, filtered and concentrated in vacuum to afford (7¹s,7³s,E)-1¹,2⁶-dimethyl-5⁶-(piperazin-1-ylmethyl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (170 mg, 92%). [M+H]⁺=527.4.

### Step 10: (R)-3-(4-(3-(4-(((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carbonyl)azetidin-1-yl)-2,6-difluorophenyl)piperidine-2,6-dione

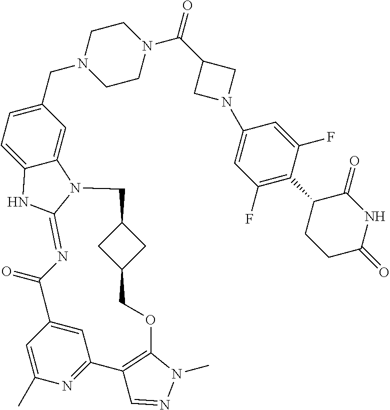 [SMILES: Cc1cc2cc(n1)-c1cnn(C)c1OC[C@H]1C[C@@H](CN3/C(=N/C2=O)Nc2ccc(CN4CCN(C(=O)C5CN(c6cc(F)c([C@H]7CCC(=O)NC7=O)c(F)c6)C5)CC4)cc23)C1]

To a solution of (7¹s,7³s,E)-1¹,2⁶-dimethyl-5⁶-(piperazin-1-ylmethyl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (50 mg, 0.10 mmol) and intermediate 3 (36.9 mg, 0.11 mmol) in DCM (5 mL) was added T₃P (90 mg, 0.14 mmol, 50% in EA) and DIEA (24.5 mg, 0.19 mmol). The reaction was stirred at rt for 1 hr. The reaction was quenched by water (5 mL) and the resulting mixture was extracted by DCM (10 mL×3), the combined organic phase was washed with brine (10 mL×3), dried over Na₂SO₄, filtered and concentrated in vacuum. The residue was purified by prep-TLC (DCM:CH₃OH=12:1) to afford (R)-3-(4-(3-(4-(((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carbonyl)azetidin-1-yl)-2,6-difluorophenyl)piperidine-2,6-dione (24 mg, 30.4%). ¹H NMR (500 MHz, DMSO) δ 12.62 (s, 1H), 10.85 (s, 1H), 8.66 (s, 1H), 7.90 (s, 1H), 7.48-7.46 (m, 2H), 7.39 (s, 1H), 7.23-7.21 (m, 1H), 6.16 (d, J=11.1 Hz, 2H), 4.88-4.46 (m, 2H), 4.04-4.01 (m, 3H), 3.89-3.84 (m, 2H), 3.82-3.76 (m, 1H), 3.72 (s, 3H), 3.59 (s, 2H), 3.50 (s, 2H), 3.43-3.40 (m, 2H), 3.00-2.82 (m, 2H), 2.81-2.75 (m, 1H), 2.56 (s, 3H), 2.55-2.52 (m, 2H), 2.49-2.44 (m, 4H), 2.43-2.29 (m, 5H), 2.11-2.03 (m, 1H), 1.99-1.84 (m, 1H); [M+H]⁺=833.6.

### Example 4: (R)-3-(4-((S)-4-(4-(((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)methyl)piperazine-1-carbonyl)-3,3-dimethylpyrrolidin-1-yl)-2,6-difluorophenyl)piperidine-2,6-dione

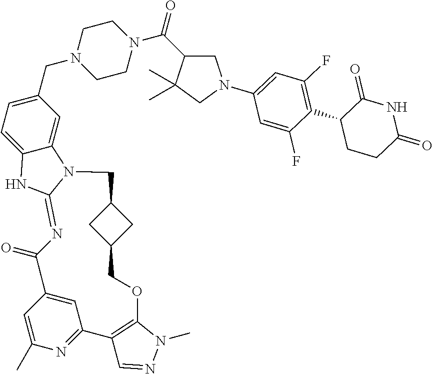 [SMILES: Cc1cc2cc(n1)-c1cnn(C)c1OC[C@H]1C[C@@H](CN3/C(=N/C2=O)Nc2ccc(CN4CCN(C(=O)C5CN(c6cc(F)c([C@H]7CCC(=O)NC7=O)c(F)c6)CC5(C)C)CC4)cc23)C1]

The titled compound was prepared in a manner similar to that in Example 3 with intermediate 4. ¹H NMR (500 MHz, DMSO) δ 12.62 (s, 1H), 10.83 (s, 1H), 8.66 (s, 1H), 7.90 (s, 1H), 7.48-7.46 (m, 2H), 7.41 (s, 1H), 7.21 (d, J=7.6 Hz, 11H), 6.14 (d, J=12.4 Hz, 2H), 4.98-4.41 (m, 2H), 4.35-4.11 (m, 2H), 4.07-3.92 (m, 1H), 3.72 (s, 3H), 3.59 (s, 4H), 3.52 (s, 2H), 3.43 (s, 2H), 3.37 (s, 1H), 3.10-3.04 (m, 2H), 2.95-2.86 (m, 3H), 2.81-2.73 (m, 1H), 2.56 (s, 3H), 2.53-2.51 (m, 1H), 2.48-2.45 (m, 2H), 2.42-2.36 (m, 5H), 2.14-2.01 (m, 1H), 1.96-1.89 (m, 1H), 1.17 (s, 3H), 0.98 (s, 3H); [M+H]⁺=875.8.

### Example 5: 3-(5-(4-(1-((7¹s,7³s,E)-1¹,2⁶-dimethyl-3-oxo-5²,5³-dihydro-1¹H,5¹H-9-oxa-4-aza-5(2,1)- benzo[d]imidazola-2(2,4)-pyridina-1(4,5)-pyrazola-7(1,3)-cyclobutanacyclononaphane-5⁶-yl)piperidin-4-yl)piperazin-1-yl)-4-methylpyridin-2-yl)piperidine-2,6-dione

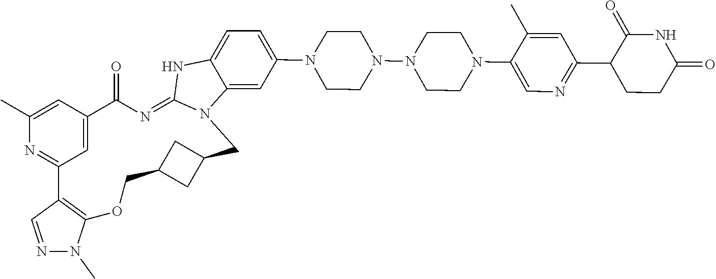 [SMILES: Cc1cc2cc(n1)-c1cnn(C)c1OC[C@H]1C[C@@H](CN3/C(=N/C2=O)Nc2ccc(N4CCN(N5CCN(c6cnc(C7CCC(=O)NC7=O)cc6C)CC5)CC4)cc23)C1]

The titled compound was prepared in a manner similar to that in Example 17. ¹H NMR (500 MHz, DMSO) δ 12.34 (s, 1H), 10.74 (s, 1H), 8.59 (s, 1H), 8.11 (s, 1H), 7.82 (s, 1H), 7.39 (s, 1H), 7.28 (d, J=8.7 Hz, 1H), 7.09 (s, 1H), 6.98 (s, 1H), 6.86 (d, J=8.8 Hz, 1H), 4.59-4.49 (m, 1H), 4.20-4.10 (m, 2H), 3.86-3.82 (m, 1H), 3.77-3.71 (m, 1H), 3.65 (s, 3H), 3.24-3.22 (m, 4H), 2.89-2.78 (m, 5H), 2.65 (t, J=11.4 Hz, 3H), 2.56 (d, J=11.2 Hz, 1H), 2.54-2.48 (m, 5H), 2.46-2.39 (m, 5H), 2.20 (s, 3H), 2.14-2.10 (m, 1H), 2.07-2.01 (m, 1H), 1.96-1.83 (m, 2H), 1.59-1.53 (m, 2H), 1.23-1.12 (m, 1H); [M+H]⁺=798.1

### Example 6: (R)-3-(4-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)piperidin-1-yl)-2,6-difluorophenyl)piperidine-2,6-dione

### Step 1: tert-butyl 4-((7¹s,7³s,E)-1¹,2⁶-dimethyl-3-oxo-5²,5³-dihydro-1H,5¹H-9-oxa-4-aza-5(2,1)-benzo[d]imidazola-2(2,4)-pyridina-1(4,5)-pyrazola-7(13)-cyclobutanacyclononaphane-5⁶-yl)piperazine-1-carboxylate

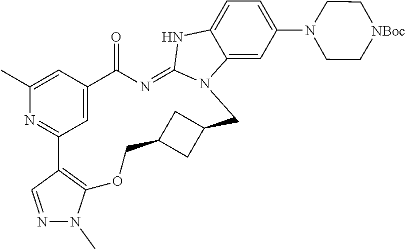 [SMILES: Cc1cc2cc(n1)-c1cnn(C)c1OC[C@H]1C[C@@H](CN3/C(=N/C2=O)Nc2ccc(N4CCN(C(=O)OC(C)(C)C)CC4)cc23)C1]

To a stirred solution of intermediate 1 (19.1 g, 102 mmol) in DMA (500 mL) were added tert-butyl piperazine-1-carboxylate (22.8 g, 122 mmol), t-BuONa (24.6 g, 256 mmol), Ruphos (19.6 g, 20 mmol) and Pd₂(dba)₃ (9.3 g, 10 mmol) at room temperature under nitrogen atmosphere. The resulting mixture was stirred for 2 h at 100° C. under nitrogen atmosphere. The mixture was allowed to cool down to room temperature. The resulting mixture was concentrated under reduced pressure. The residue was diluted with EtOAc (2000 mL), and washed with water (3×1000 mL) and brine (1000 mL). The organic layer was dried over anhydrous Na₂SO₄, filtered and concentrated under reduced pressure. The residue was purified by silica gel column chromatography, eluted with MeOH/DCM (0-7%) to afford the product (25 g, 79.6%); [M+H]⁺=613.6.

### Step 2: (7 s,7³s,E)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

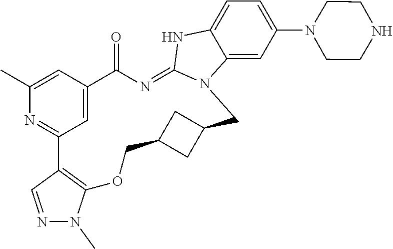 [SMILES: Cc1cc2cc(n1)-c1cnn(C)c1OC[C@H]1C[C@@H](CN3/C(=N/C2=O)Nc2ccc(N4CCNCC4)cc23)C1]

To a solution of tert-butyl 4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazine-1-carboxylate (25 g, 40.8 mmol) in DCM (100 mL) was added TFA (50 mL). The reaction was stirred at room temperature for 2 hr and then concentrated in vacuo. The residue was dissolved in DCM (1500 mL), washed with sat. aq. NaHCO₃ (3×500 mL) and brine (500 mL), dried over Na₂SO₄, filtered and concentrated under reduced pressure. The residue was purified by silica gel column chromatography, eluted with MeOH (with 3% NH₃·H₂O)/DCM (0-35%) to afford the product (16 g, 76.5%); [M+H]⁺=513.5.

### Step 3: (R)-3-(4-(4-(4-((7¹s,7³s,E)-1¹,2⁶-dimethyl-3-oxo-5²,5³-dihydro-1H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)piperidin-1-yl)-2,6-difluorophenyl)piperidine-2,6-dione

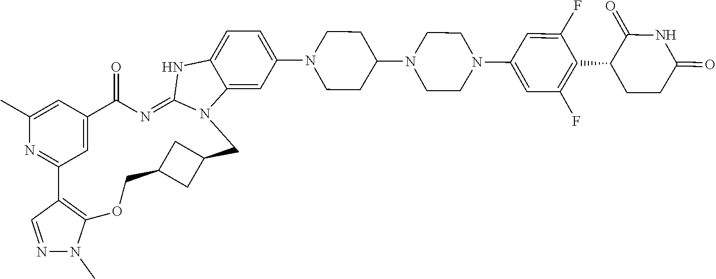 [SMILES: Cc1cc2cc(n1)-c1cnn(C)c1OC[C@H]1C[C@@H](CN3/C(=N/C2=O)Nc2ccc(N4CCC(N5CCN(c6cc(F)c([C@H]7CCC(=O)NC7=O)c(F)c6)CC5)CC4)cc23)C1]

To a solution of (7¹s,7³s,E)-ji,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (150 mg, 0.3 mmol), and intermediate 6 (188 mg, 0.6 mmol) in DCE (8 mL) was added STAB (127 mg, 0.6 mmol). Then the mixture was stirred at 50° C. for 16 hr. Sat. aq. NaHCO₃ (70 mL) was added and the resulting mixture was extracted with DCM (30 mL×3). The combined organic phase was washed with brine (50 mL), dried over Na₂SO₄, filtered and concentrated in vacuum. The residue was purified by silica gel column (MeOH:DCM=0-10%) to afford the product (130 mg, 54.3%).

¹H NMR (500 MHz, DMSO) δ 12.41 (s, 1H), 10.85 (s, 1H), 8.65 (s, 1H), 7.89 (s, 1H), 7.46 (s, 1H), 7.35 (d, J=8.9 Hz, 1H), 7.01 (s, 1H), 6.89 (d, J=9.6 Hz, 1H), 6.64 (d, J=13.0 Hz, 2H), 4.72-4.24 (m, 4H), 4.05 (d, J=7.9 Hz, 1H), 3.81 (d, J=11.9 Hz, 2H), 3.71 (s, 3H), 3.15 (s, 4H), 2.91 (s, 3H), 2.75-2.77 (m, 4H), 2.63-2.68 (m, 4H), 2.55-2.54 (m, 4H), 2.36 (s, 2H), 2.10-2.08 (m, 2H), 1.97 (s, 1H), 1.86-1.89 (m, 2H), 1.50-1.48 (m, 2H); [M+H]⁺=819.6.

### Example 7: 3-(5-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)pyridin-2-yl)piperidine-2,6-dione

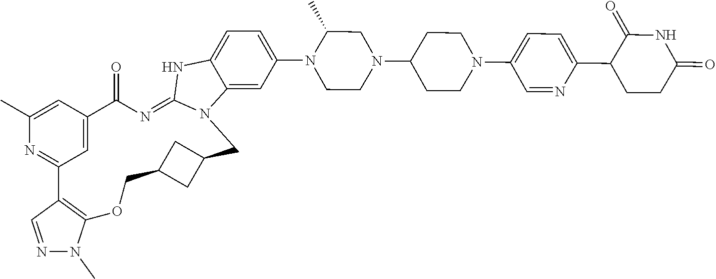 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C[C@H]4C)cc23)C1]

The titled compound was prepared in a manner similar to that in Example 33.

¹H NMR (500 MHz, DMSO) δ 12.34 (s, 1H), 10.71 (s, 1H), 8.59 (s, 1H), 8.16 (s, 1H), 7.82 (s, 1H), 7.39 (s, 1H), 7.28 (t, J=8.3 Hz, 2H), 7.10 (d, J=8.7 Hz, 1H), 6.94 (s, 1H), 6.81 (d, J=7.7 Hz, 1H), 4.78-3.93 (m, 4H), 3.90-3.79 (m, 2H), 3.77-3.67 (m, 2H), 3.65 (s, 3H), 3.23-3.22 (m, 6H), 2.93 (s, 1H), 2.83 (s, 3H), 2.70 (t, J=11.6 Hz, 2H), 2.61 (s, 1H), 2.58-2.55 (m, 1H), 2.53-2.47 (m, 6H), 2.14-2.08 (m, 1H), 2.05-2.01 (m, 1H), 1.84 (s, 2H), 1.50 (s, 2H), 0.90 (d, J=5.9 Hz, 3H); [M+H]⁺=798.5

### Example 9: (R)-3-(4-(2-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-oxopiperazin-1-yl)ethyl)-2,6-difluorophenyl)piperidine-2,6-dione

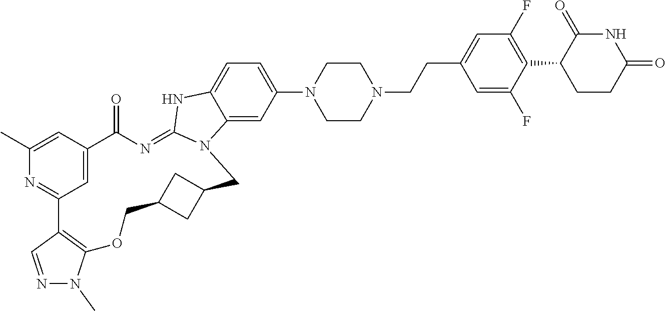 [SMILES: Cc1cc2cc(n1)-c1cnn(C)c1OC[C@H]1C[C@@H](CN3/C(=N/C2=O)Nc2ccc(N4CCN(CCc5cc(F)c([C@H]6CCC(=O)NC6=O)c(F)c5)CC4)cc23)C1]

The titled compound was prepared in a manner similar to that in Example 22 with intermediate 5. ¹H NMR (500 MHz, DMSO) δ 12.67 (s, 1H), 10.96 (s, 1H), 8.66 (s, 1H), 7.90 (s, 1H), 7.55 (d, J=1.4 Hz, 1H), 7.50 (s, 1H), 7.48 (s, 1H), 7.18 (dd, J=8.5, 1.7 Hz, 1H), 7.09 (d, J=10.0 Hz, 2H), 4.62 (s, 2H), 4.40-3.94 (m, 3H), 3.72 (s, 3H), 3.69 (t, J=5.2 Hz, 2H), 3.30-2.25 (m, 4H), 2.92-2.78 (m, 6H), 2.73 (t, J=7.2 Hz, 2H), 2.60-2.51 (m, 7H), 2.14 (dt, J=12.9, 10.9 Hz, 1H), 2.05-1.96 (m, 1H). [M+H]⁺=778.6.

### Example 10: (R)-3-(4-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-oxopiperazin-1-yl)piperidin-1-yl)-2,6-difluorophenyl)piperidine-2,6-dione

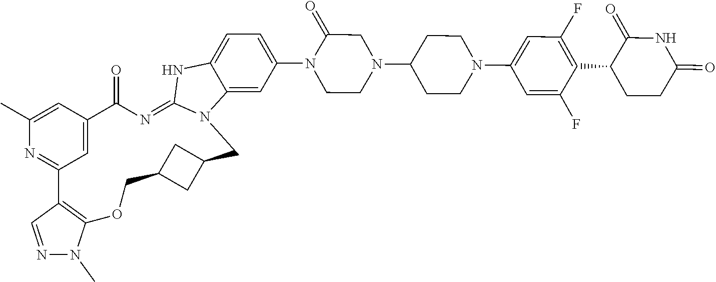 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)CC4=O)cc23)C1]

The titled compound was prepared in a manner similar to that in Example 22 with intermediate 6. ¹H NMR (500 MHz, DMSO) δ 12.67 (s, 1H), 10.87 (s, 1H), 8.66 (s, 1H), 7.89 (s, 1H), 7.54 (s, 1H), 7.49 (d, J=8.8 Hz, 1H), 7.48 (s, 1H), 7.17 (dd, J=8.5, 1.6 Hz, 1H), 6.66 (d, J=12.8 Hz, 2H), 4.87-4.48 (m, 2H), 4.05 (dd, J=12.7, 5.1 Hz, 3H), 3.81 (d, J=12.5 Hz, 2H), 3.72 (s, 3H), 3.67 (s, 2H), 3.34 (s, 2H), 3.31-3.24 (m, 2H), 2.90 (s, 3H), 2.79 (t, J=11.4 Hz, 4H), 2.59-2.53 (m, 7H), 2.13-2.05 (m, 1H), 2.01-1.94 (m, 1H), 1.91 (d, J=10.1 Hz, 2H), 1.55-1.43 (m, 2H). [M+H]⁺=833.7.

### Example 11: (R)-3-(4-(3-((4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-oxopiperazin-1-yl)methyl)azetidin-1-yl)-2,6-difluorophenyl)piperidine-2,6-dione

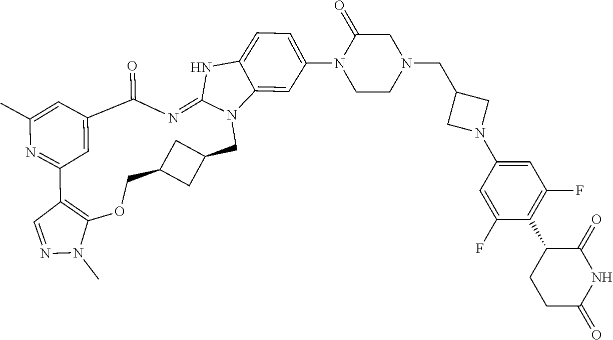 [SMILES: Cc1cc2cc(n1)-c1cnn(C)c1OC[C@H]1C[C@@H](CN3/C(=N/C2=O)Nc2ccc(N4CCN(CC5CN(c6cc(F)c([C@H]7CCC(=O)NC7=O)c(F)c6)C5)CC4=O)cc23)C1]

The titled compound was prepared in a manner similar to that in Example 22 with intermediate 8. ¹H NMR (500 MHz, DMSO) δ 12.66 (s, 1H), 10.86 (s, 1H), 8.66 (s, 1H), 7.90 (s, 1H), 7.55 (s, 1H), 7.49 (d, J=8.6 Hz, 1H), 7.48 (s, 1H), 7.17 (d, J=8.4 Hz, 1H), 6.14 (d, J=11.2 Hz, 2H), 4.67 (s, 2H), 4.06 (s, 2H), 4.03 (dd, J=12.7, 4.9 Hz, 1H), 3.97 (s, 2H), 3.72 (s, 3H), 3.69 (s, 2H), 3.54 (s, 2H), 3.21 (s, 2H), 3.04-2.97 (m, 1H), 2.91 (s, 2H), 2.84 (s, 2H), 2.81-2.76 (m, 1H), 2.73 (d, J=7.5 Hz, 2H), 2.60-2.51 (m, 8H), 2.11-2.04 (m, 1H), 1.99-1.92 (m, 1H). [M+H]⁺=819.7.

### Example 12: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-oxopiperazin-1-yl)piperidin-1-yl)pyridin-2-yl)piperidine-2,6-dione

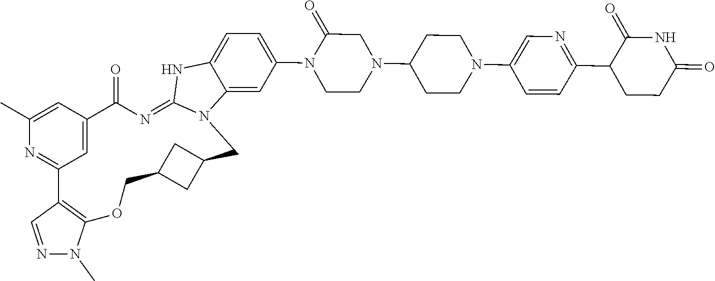 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)CC4=O)cc23)C1]

The titled compound was prepared in a manner similar to that in Example 22 with intermediate 11. ¹H NMR (500 MHz, DMSO) δ 12.67 (s, 1H), 10.78 (s, 1H), 8.66 (s, 1H), 8.23 (d, J=2.8 Hz, 1H), 7.90 (s, 1H), 7.55 (s, 1H), 7.49 (d, J=8.6 Hz, 1H), 7.48 (s, 1H), 7.35 (dd, J=8.6, 2.8 Hz, 1H), 7.17 (d, J=8.7 Hz, 2H), 4.55 (s, 2H), 4.21 (s, 2H), 3.89 (dd, J=8.8, 5.3 Hz, 1H), 3.79 (d, J=11.5 Hz, 2H), 3.72 (s, 3H), 3.68 (s, 2H), 3.35 (s, 2H), 2.92 (s, 4H), 2.76 (t, J=11.4 Hz, 2H), 2.65-2.52 (m, 10H), 2.19 (dt, J=18.4, 7.2 Hz, 1H), 2.10 (dd, J=13.1, 6.0 Hz, 1H), 1.95 (d, J=12.1 Hz, 2H), 1.57 (d, J=10.2 Hz, 2H).[M+H]⁺=798.8.

### Example 14: (R)-3-(4-((2-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)ethyl)amino)-2,6-difluorophenyl)piperidine-2,6-dione

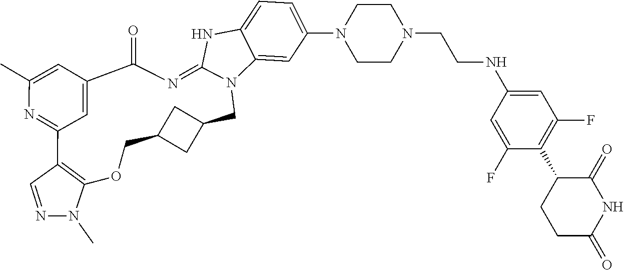 [SMILES: Cc1cc2cc(n1)-c1cnn(C)c1OC[C@H]1C[C@@H](CN3/C(=N/C2=O)Nc2ccc(N4CCN(CCNc5cc(F)c([C@H]6CCC(=O)NC6=O)c(F)c5)CC4)cc23)C1]

A solution of (7 s,7³s,E)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (80 mg, 0.16 mmol), intermediate 13 (74 mg, 0.2 mmol), DIEA (61 mg, 0.47 mmol) and KI (52 mg, 0.31 mmol) in MeCN (5 mL) and DMSO (1 mL) was stirred at 80° C. for 16 hr. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=15:1), followed by SFC (Column: CHIRALPAK IE-3, 4.6*50 mm, 3 mm; MtBE (0.1% FA):(MeOH:DCM=1:1)=35:65) and the title compound corresponded to peak A @2.222 min/254 nm (6.2 mg, 5.1%). ¹H NMR (500 MHz, DMSO) δ 12.41 (s, 1H), 10.83 (s, 1H), 8.65 (s, 1H), 7.89 (s, 1H), 7.46 (s, 111), 7.35 (d, J=8.7 Hz, 1H), 7.03 (s, 111), 6.91 (d, J=8.3 Hz, 1H), 6.29 (d, J=12.1 Hz, 2H), 6.11 (s, 1H), 4.78-4.43 (m, 4H), 4.00-3.97 (m, 1H), 3.72 (s, 3H), 3.32 (s, 3H), 3.20-3.18 (m, 6H), 2.91 (s, 3H), 2.81-2.73 (m, 1H), 2.64-2.61 (m, 5H), 2.57-2.54 (m, 5H), 2.10-2.02 (m, 1H), 1.98-1.91 (m, 1H); [M+H]⁺=779.6.

### Example 15: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)piperidin-1-yl)pyridin-2-yl)piperidine-2,6-dione

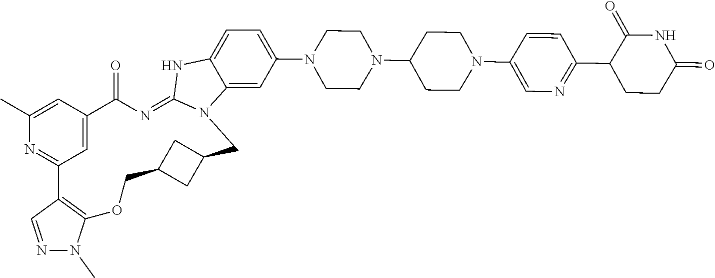 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)CC4)cc23)C1]

The titled compound was prepared in a manner similar to that in Example 21 with intermediate 11. ¹H NMR (500 MHz, DMSO) δ 12.40 (s, 1H), 10.78 (s, 1H), 8.65 (s, 1H), 8.22 (d, J=2.8 Hz, 1H), 7.89 (s, 1H), 7.46 (s, 1H), 7.33 (dd, J=11.6, 5.8 Hz, 2n), 7.16 (d, J=8.6 Hz, 1H), 7.01 (s, 1H), 6.89 (dd, J=8.8, 1.6 Hz, 1H), 4.94-4.39 (m, 2H), 4.36-3.93 (m, 2H), 3.9-3.87 (m, 1H), 3.8-3.77 (m, 2H), 3.72 (s, 3H), 3.16 (s, 5H), 2.93-2.98 (m, 3H), 2.77-2.59 (m, 7H), 2.59-2.51 (m, 6H), 2.44-2.40 (m, 1H), 2.22-2.15 (m, 1H), 2.13-2.07 (m, 1H), 1.91 (d, J=11.5 Hz, 2H), 1.58-1.52 (m, 2H); [M+H]-784.6.

### Example 16: (R)-3-(4-(((1S,3S)-3-(4-((7¹s,7³S,E)-1¹,2⁶-dimethyl-3-oxo-5²,5³-dihydro-1 ¹H,5¹H-9- oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)cyclobutyl)amino)-2,6-difluorophenyl)piperidine-2,6-dione

### Step 1: tert-butyl 4-(3-(((benzyloxy)carbonyl)amino)cyclobutyl)piperazine-1-carboxylate

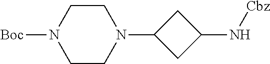 [SMILES: CC(C)(C)OC(=O)N1CCN(C2CC(NC(=O)OCc3ccccc3)C2)CC1]

To a 250-mL round-bottomed flask equipped with a magnetic stir bar was added benzyl (3-oxocyclobutyl)carbamate (2.2 g, 10.0 mmol), tert-butyl piperazine-1-carboxylate (2.2 g, 12.0 mmol), NaBH(OAc)₃ (4.2 g, 20.0 mmol) and DCE (100 mL). After stirring for 6 h at 50° C., the reaction mixture was diluted with sat. aq. NaHCO₃ (150 mL) and the layers were separated. The aqueous layer was extracted with DCM (3×70 mL). The combined organic layers were washed with brine (150 mL), dried over anhydrous Na₂SO₄, filtered and concentrated under reduced pressure. Purification by flash column chromatography (0-7% MeOH in DCM) afforded the product (3.0 g, 77%). [M+H]⁺=390.0.

### Step 2: tert-butyl 4-(3-aminocyclobutyl)piperazine-1-carboxylate

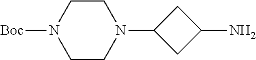 [SMILES: CC(C)(C)OC(=O)N1CCN(C2CC(N)C2)CC1]

To the solution of tert-butyl 4-(3-(((benzyloxy)carbonyl)amino)cyclobutyl)piperazine-1-carboxylate (3.8 g, 9.8 mmol) in 100 mL MeOH was added Pd/C (4.0 g, 10 wt. %, wet). The mixture was stirred at rt for 3 hours under hydrogen atmosphere (balloon). The mixture was filtered by celite directly, and the solid was washed by MeOH (20 mL) and DCM (100 mL). The filtrate was concentrated in vacuum to afford the crude product (2.8 g), which was used without further purification. [M+H]⁺=255.9.

### Step 3: tert-butyl 4-((1s,3s)-3-((4-(2,6-bis(benzyloxy)pyridin-3-yl)-3,5-difluorophenyl)amino)cyclobutyl)piperazine-1-carboxylate

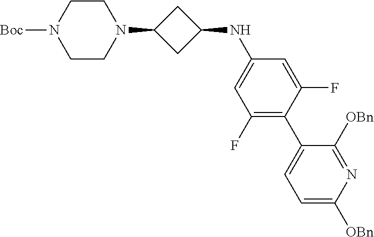 [SMILES: CC(C)(C)OC(=O)N1CCN([C@H]2C[C@@H](Nc3cc(F)c(-c4ccc(OCc5ccccc5)nc4OCc4ccccc4)c(F)c3)C2)CC1]

The solution of intermediate 16 (3.5 g, 7.2 mmol), tert-butyl 4-(3-aminocyclobutyl)piperazine-1-carboxylate (2.8 g, 10.8 mmol), K₃PO₄ (5.1 g, 24.0 mmol), CuI (306 mg, 1.6 mmol) and L-Proline (522 mg, 3.2 mmol) in 100 mL DMSO was stirred at 100° C. for 16 hours under nitrogen atmosphere. The mixture was diluted with EtOAc and filtered. The filtrate was washed with brine (300 mL×3), dried over anhydrous Na₂SO₄, filtered and concentrated under reduced pressure. The crude was purified by flash column chromatography (0-7% MeOH in DCM) and SFC (Lux Cellulose-2 4.6*50 mm 3 um, Hex:EtOH=95:5) and the title compound corresponded to peak A @2.692 min/254 nm (384 mg, 8%). [M+H]657.1.

### Step 4: tert-butyl 4-((1s,3s)-3-((4-(2,6-dioxopiperidin-3-yl)-3,5-difluorophenyl)amino)cyclobutyl)piperazine-1-carboxylate

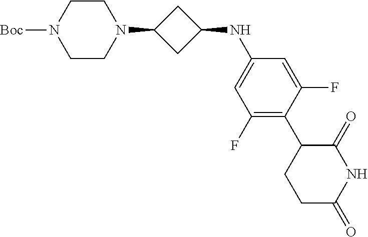 [SMILES: CC(C)(C)OC(=O)N1CCN([C@H]2C[C@@H](Nc3cc(F)c(C4CCC(=O)NC4=O)c(F)c3)C2)CC1]

To the solution of tert-butyl 4-((1s,3s)-3-((4-(2,6-bis(benzyloxy)pyridin-3-yl)-3,5-difluorophenyl)amino)cyclobutyl)piperazine-1-carboxylate (1.0 g, 1.5 mmol) in 50 mL DMF/iPrOH was added Pd/C (1.0 g, 10% w.t.). The mixture was stirred at 50° C. for 16 hours under H₂ atmosphere. The mixture was filtered through celite directly, and the solid was washed by MeOH (20 mL) and DCM (100 mL). The filtrate was concentrated in vacuum to afford the crude product (604 mg), which was used without further purification. [M+H]⁺=478.9.

### Step 5: 3-(2,6-difluoro-4-(((1s,3s)-3-(piperazin-1-yl)cyclobutyl)amino)phenyl)piperidine-2,6-dione

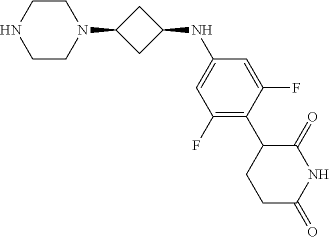 [SMILES: O=C1CCC(c2c(F)cc(N[C@H]3C[C@@H](N4CCNCC4)C3)cc2F)C(=O)N1]

To a 100-mL round-bottomed flask equipped with a magnetic stir bar was added tert-butyl 4-((1s,3s)-3-((4-(2,6-dioxopiperidin-3-yl)-3,5-difluorophenyl)amino)cyclobutyl)piperazine-1-carboxylate (604 mg, 1.3 mmol), DCM (40 mL) and TFA (10 mL). After stirring for 1 h at room temperature, the reaction mixture was concentrated under reduced pressure to afforded the product as TFA salt (720 mg), which was used without further purification. [M+H]⁺=378.9.

### Step 6: (R)-3-(4-(((1S,3S)-3-(4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)cyclobutyl)amino)-2,6-difluorophenyl)piperidine-2,6-dione

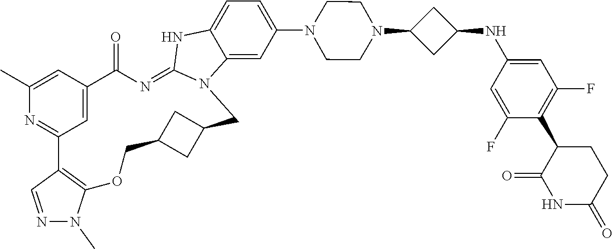 [SMILES: Cc1cc2cc(n1)-c1cnn(C)c1OC[C@H]1C[C@@H](CN3/C(=N/C2=O)Nc2ccc(N4CCN([C@H]5C[C@@H](Nc6cc(F)c([C@H]7CCC(=O)NC7=O)c(F)c6)C5)CC4)cc23)C1]

To a stirred solution of intermediate 1 (110 mg, 0.22 mmol) and 3-(2,6-difluoro-4-(((1s,3s)-3-(piperazin-1-yl)cyclobutyl)amino)phenyl)piperidine-2,6-dione (TFA salt) (150 mg, 0.33 mmol) in DMA (10 mL) were added t-BuONa (127 mg, 1.32 mmol), Ruphos (41 mg, 0.088 mmol) and Pd₂(dba)₃ (40 mg, 0.044 mmol) at room temperature under nitrogen atmosphere. The resulting mixture was stirred for 2 h at 100° C. under nitrogen atmosphere. The mixture was allowed to cool down to room temperature. The resulting mixture was concentrated under reduced pressure. The residue was diluted with DCM (40 mL), washed with sat. aq. NH₄Cl (2×30 mL) and brine (50 mL). The organic layer was dried over anhydrous Na₂SO₄, filtered and concentrated under reduced pressure. The residue was purified by silica gel column chromatography, eluted with DCM/MeOH (10:1), followed by SFC (Column: CHIRALPAK IA, 2 cm×25 cm, 5 um; Flow rate: 20 mL/min;) and the title compound corresponded to peak A @2.227 min/254 nm (4.32 mg, 2.5%). ¹H NMR (500 MHz, DMSO) δ 12.41 (s, 1H), 10.84 (s, 1H), 8.66 (s, 1H), 7.89 (s, 1H), 7.46 (s, 1H), 7.35 (d, J=8.8 Hz, 1H), 7.02 (s, 1H), 6.91 (d, J=9.1 Hz, 1H), 6.50 (d, J=7.4 Hz, 1H), 6.18 (d, J=11.6 Hz, 2H), 3.99-3.96 (m, 2H), 3.72 (s, 4H), 3.19-3.14 (m, 3H), 2.95-2.88 (m, 5H), 2.67-2.62 (m, 5H), 2.59-2.52 (m, 6H), 2.46-2.43 (m, 3H), 2.37 (s, 2H), 2.09-2.01 (m, 1H), 1.98-1.91 (m, 1H), 1.72-1.65 (m, 2H), 1.24 (s, 1H); [M+H]⁺=805.4

### Example 17: (R)-3-(4-(4-(1-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperidin-4-yl)piperazin-1-yl)-2,6-difluorophenyl)piperidine-2,6-dione

### Step 1: (7¹s,7³s,E)-1¹,2⁶-dimethyl-5⁶-(1,4-dioxa-8-azaspiro[4.5]decan-8-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

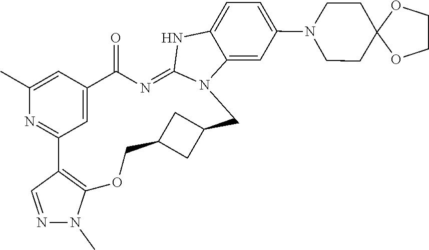 [SMILES: Cc1cc2cc(n1)-c1cnn(C)c1OC[C@H]1C[C@@H](CN3/C(=N/C2=O)Nc2ccc(N4CCC5(CC4)OCCO5)cc23)C1]

To the solution of intermediate 1 (3.0 g, 5.9 mmol), 1,4-dioxa-8-azaspiro[4.5]decane (1.7 g, 11.8 mmol) and t-BuONa (1.2 g, 11.8 mmol) in 80 mL DMA, were added Pd₂(dba)₃ (540 mg, 0.59 mmol) and RuPhos (550 mg, 1.18 mmol). The mixture was stirred at 100° C. for 3 hours under N₂. After LCMS showed the reaction was completed, the mixture was diluted with water and extracted by EtOAc. The organic layer was washed with brine, dried over anhydrous Na₂SO₄, filtered and concentrated under reduced pressure. The resulting mixture was purified by silica column chromatography (DCM:MeOH=100:1-15:1) to afford the product (2.5 g, 73.2%). [M+H]⁺=570.4.

### Step 2: (7¹s,7³s,E)-1¹,2⁶-dimethyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

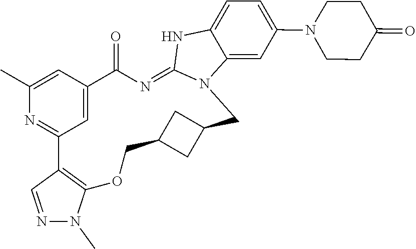 [SMILES: Cc1cc2cc(n1)-c1cnn(C)c1OC[C@H]1C[C@@H](CN3/C(=N/C2=O)Nc2ccc(N4CCC(=O)CC4)cc23)C1]

(7¹s,7³s,E)-1¹,2⁶-dimethyl-5⁶-(1,4-dioxa-8-azaspiro[4.5]decan-8-yl)-5²,5³-dihydro-1¹H,5¹H-9-oxa-4-aza- 5(2,1)-benzo[d]imidazola-2(2,4)-pyridina-1(4,5)-pyrazola-7(1,3)-cyclobutanacyclononaphan-3-one (300 mg, 0.53 mmol) was placed in 25 mL round bottom flask with a magnetic stir bar. Then, 4 mL concentrated HCl aqueous was added. The mixture was stirred at room temperature for 2 hours. The mixture was added dropwise to sat. aq. NaHCO₃ solution and pH was adjusted to 6-7. The liquid was extracted with DCM. The combined organic phase was dried over anhydrous Na₂SO₄, concentrated in vacuum and purified with combiflash (DCM:MeOH=20:1) to afford the title compound (120 mg, 43.2% yield). [M+H]⁺=526.5.

### Step 3: (R)-3-(4-(4-(1-((7¹s,7³s,E)-1¹,2⁶-dimethyl-3-oxo-5²,5³-dihydro-1′H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperidin-4-yl)piperazin-1-yl)-2,6-difluorophenyl)piperidine-2,6-dione

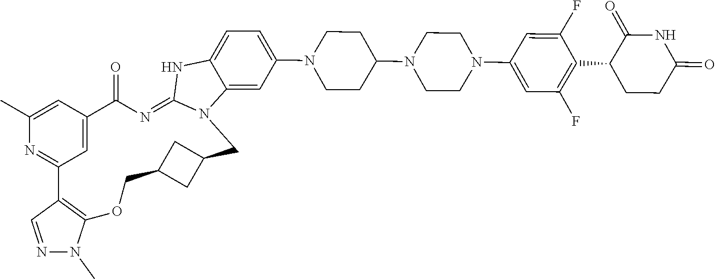 [SMILES: Cc1cc2cc(n1)-c1cnn(C)c1OC[C@H]1C[C@@H](CN3/C(=N/C2=O)Nc2ccc(N4CCC(N5CCN(c6cc(F)c([C@H]7CCC(=O)NC7=O)c(F)c6)CC5)CC4)cc23)C1]

To a solution of (7¹s,7³s,E)-1¹,2⁶-dimethyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (100 mg, 0.19 mmol), intermediate 14 (86 mg, 0.25 mmol) and DIEA (50 mg, 0.38 mmol) in DCE (8 mL) was added STAB (121 mg, 0.57 mmol). Then the mixture was stirred at 50° C. for 16 hours. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=10:1), followed by Prep-HPLC chromatography to afford the title product (30 mg, 14.7%). ¹H NMR (500 MHz, DMSO) δ 12.41 (s, 1H), 10.88 (s, 1H), 8.66 (s, 1H), 7.90 (s, 1H), 7.46 (s, 1H), 7.34 (d, J=8.7 Hz, 1H), 7.03 (s, 1H), 6.92 (d, J=8.8 Hz, 1H), 6.65 (d, J=12.6 Hz, 2H), 4.59-4.52 (m, 2H), 4.19-4.13 (m, 1H), 4.06 (dd, J=12.5, 5.0 Hz, 1H), 3.75 (d, J=8.8 Hz, 2H), 3.72 (s, 3H), 3.32-3.28 (m, 5H), 3.20 (s, 4H), 2.94-2.89 (m, 3H), 2.83-2.67 (m, 7H), 2.56 (s, 3H), 2.43-2.33 (m, 1H), 2.09 (dd, J=13.0, 3.8 Hz, 1H), 2.02-1.91 (m, 3H), 1.64-1.59 (m, 2H); [M+H]⁺=819.6.

### Example 18: (R)-3-(4-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)-2,6-difluorophenyl)piperidine-2,6-dione

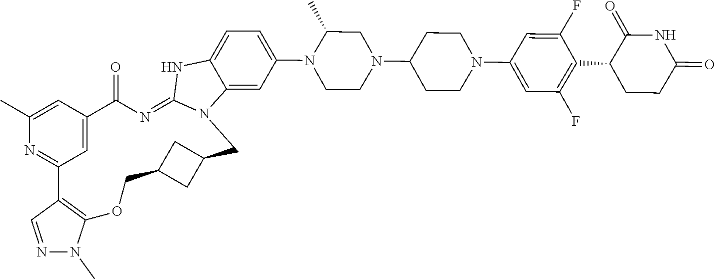 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)C[C@H]4C)cc23)C1]

The titled compound was prepared in a manner similar to that in Example 33 with intermediate 6. ¹H NMR (500 MHz, DMSO) δ 12.40 (s, 1H), 10.87 (s, 1H), 8.65 (s, 1H), 7.89 (s, 1H), 7.46 (s, 1H), 7.35 (d, J=8.7 Hz, 1H), 7.00 (s, 1H), 6.87 (d, J=9.0 Hz, 1H), 6.64 (d, J=12.8 Hz, 2H), 5.11-4.12 (m, 4H), 4.07-4.03 (m, 1H), 3.90 (s, 1H), 3.81-3.79 (m, 2H), 3.72 (s, 3H), 3.29 (s, 1H), 3.23-3.22 (m, 1H), 3.00-2.97 (m, 5H), 2.82-2.77 (m, 3H), 2.71-2.53 (m, 9H), 2.44 (s, 1H), 2.09 (d, J=16.5 Hz, 1H), 1.96 (d, J=5.8 Hz, 1H), 1.85 (s, 2H), 1.52-1.49 (m, 2H), 0.96 (d, J=6.3 Hz, 3H); [M+H]⁺=833.6.

### Example 19: 3-(5-((3R,4S)-4-(4-((7¹s,7³R,E)-1¹,2⁶-dimethyl-3-oxo-5²,5³-dihydro-1¹H,5¹H-9-oxa-4- aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-6-methylpyridin-2-yl)piperidine-2,6-dione

### Step 1: tert-butyl (3R,4S)-4-(4-benzylpiperazin-1-yl)-3-fluoropiperidine-1-carboxylate

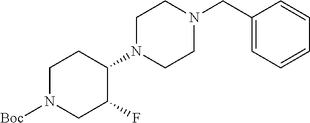 [SMILES: CC(C)(C)OC(=O)N1CC[C@H](N2CCN(Cc3ccccc3)CC2)[C@H](F)C1]

To the solution of tert-butyl (3R,4S)-4-amino-3-fluoropiperidine-1-carboxylate (2.5 g, 11.46 mmol) N-benzyl-2-chloro-N-(2-chloroethyl)ethan-1-amine hydrochloride (3 g, 11.46 mmol) and NaHCO₃ (3.85 g, 45.84 mmol) in 50 mL EtOH, the mixture was stirred at 80° C. for 16 hours. After LCMS shown the reaction was completed. The mixture was concentrated, washed with water and extracted by DCM. The organic layer was separated and concentrated. The mixture was purified by silica column chromatography (MeOH:DCM=0-4%) to afford tert-butyl (3R,4S)-4-(4-benzylpiperazin-1-yl)-3-fluoropiperidine-1-carboxylate (2 g, 5.3 mmol, 46.3%). [M+H]⁺=378.6.

### Step 2: 1-benzyl-4-((3R,4S)-3-fluoropiperidin-4-yl)piperazine

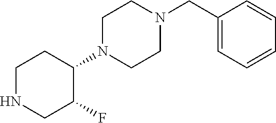 [SMILES: F[C@@H]1CNCC[C@@H]1N1CCN(Cc2ccccc2)CC1]

To the solution of tert-butyl (3R,4S)-4-(4-benzylpiperazin-1-yl)-3-fluoropiperidine-1-carboxylate (2 g, 5.3 mmol) in 2 mL DCM, 6 mL TFA was added. The mixture was stirred at room temperature for 1 hour. The mixture was concentrated and sat. aq. NaHCO₃ was added. The water layer was extracted with DCM and separated. The organic layer was dried with Na₂SO₄ was filtered. The liquid was concentrated to afford 1-benzyl-4-((3R,4S)-3-fluoropiperidin-4-yl)piperazine (1.1 g, 3.97 mmol, 74.93%). [M+H]⁺=278.6.

### Step 3: 2′,6′-bis(benzyloxy)-5-((3R,4S)-4-(4-benzylpiperazin-1-yl)-3-fluoropiperidin-1-yl)-6-methyl-2,3′-bipyridine

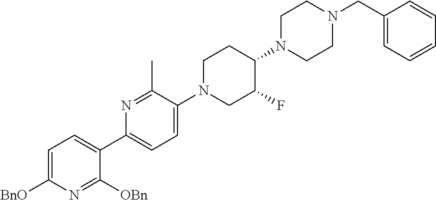 [SMILES: Cc1nc(-c2ccc(OCc3ccccc3)nc2OCc2ccccc2)ccc1N1CC[C@H](N2CCN(Cc3ccccc3)CC2)[C@H](F)C1]

To the solution of 1-benzyl-4-((3R,4S)-3-fluoropiperidin-4-yl)piperazine (1.1 g, 3.97 mmol), 2′,6′-bis(benzyloxy)-5-bromo-6-methyl-2,3′-bipyridine (1.84 g, 4 mmol) and Cs₂CO₃ (2.61 g, 8 mmol) in 15 mL DMA, Pd₂(dba)₃ (915 mg, 1 mmol) and Ruphos (466 mg, 1 mmol) was added. The mixture was stirred at 100° C. for 16 hours under N₂ protected. The mixture was concentrated in vacuum. The residue was washed with saturated NaCl aqueous and extracted with EtOAc. The organic layer was separated and concentrated. The residue was purified by silica column chromatography (EA:PE=0-100%) to afford 2′,6′-bis(benzyloxy)-5-((3R,4S)-4-(4-benzylpiperazin-1-yl)-3-fluoropiperidin-1-yl)-6-methyl-2,3′-bipyridine (1.2 g, 1.83 mmol, 46%). [M+H]⁺=658.7.

### Step 4: tert-butyl 4-((3R,4S)-1-(6-(2,6-dioxopiperidin-3-yl)-2-methylpyridin-3-yl)-3-fluoropiperidin-4-yl)piperazine-1-carboxylate

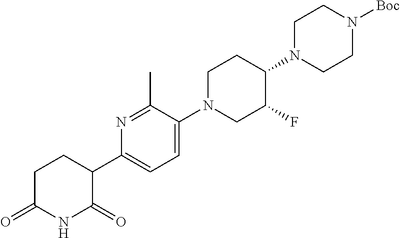 [SMILES: Cc1nc(C2CCC(=O)NC2=O)ccc1N1CC[C@H](N2CCN(C(=O)OC(C)(C)C)CC2)[C@H](F)C1]

To the solution of 2′,6′-bis(benzyloxy)-5-((3R,4S)-4-(4-benzylpiperazin-1-yl)-3-fluoropiperidin-1-yl)-6-methyl-2,3′-bipyridine (1.2 g, 1.83 mmol) in 40 mL DMF and 40 mL iPrOH, 2 mL Boc₂O and Pd/C (1.2 g, 10% w.t.) was added. The mixture was stirred at 55° C. for 16 hours at H₂ atmosphere. The mixture was filtered by celite. The liquid was concentrated and purified by silica column chromatography (MeOH:DCM=0-7%) to afford tert-butyl 4-((3R,4S)-1-(6-(2,6-dioxopiperidin-3-yl)-2-methylpyridin-3-yl)-3-fluoropiperidin-4-yl)piperazine-1-carboxylate (450 mg, 0.92 mmol, 50.27%). [M+H]⁺=490.7.

### Step 5: 3-(5-((3R,4S)-3-fluoro-4-(piperazin-1-yl)piperidin-1-yl)-6-methylpyridin-2-yl)piperidine-2,6-dione 2,2,2-trifluoroacetate

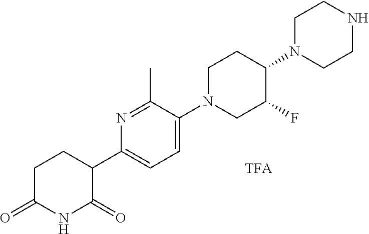 [SMILES: Cc1nc(C2CCC(=O)NC2=O)ccc1N1CC[C@H](N2CCNCC2)[C@H](F)C1]

To the solution of tert-butyl 4-((3R,4S)-1-(6-(2,6-dioxopiperidin-3-yl)-2-methylpyridin-3-yl)-3-fluoropiperidin-4-yl)piperazine-1-carboxylate (150 mg, 0.31 mmol) in 1 mL DCM, 3 mL TFA was added. The mixture was stirred at room temperature for 1 hour. After LCMS shown the reaction was completed. The mixture was concentrated to afford 3-(5-((3R,4S)-3-fluoro-4-(piperazin-1-yl)piperidin-1-yl)-6-methylpyridin-2-yl)piperidine-2,6-dione 2,2,2-trifluoroacetate (120 mg crude). [M+H]⁺=390.7.

### Step 6: 3-(5-((3R,4S)-4-(4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-6-methylpyridin-2-yl)piperidine-2,6-dione

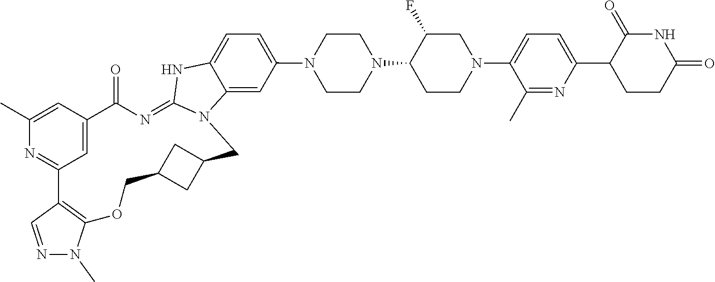 [SMILES: Cc1cc2cc(n1)-c1cnn(C)c1OC[C@H]1C[C@@H](CN3/C(=N/C2=O)Nc2ccc(N4CCN([C@H]5CCN(c6ccc(C7CCC(=O)NC7=O)nc6C)C[C@H]5F)CC4)cc23)C1]

The titled compound was prepared in a manner similar to that in Example 26 step 4. ¹H NMR (500 MHz, DMSO) δ 12.35 (s, 1H), 10.72 (s, 1H), 8.59 (s, 1H), 7.83 (s, 1H), 7.40 (s, 1H), 7.34-7.26 (m, 2H), 7.04 (d, J=8.1 Hz, 1H), 6.95 (s, 1H), 6.84 (d, J=8.8 Hz, 1H), 5.09-4.96 (m, 1H), 4.65-3.95 (m, 4H), 3.87-3.83 (m, 1H), 3.65 (s, 3H), 3.15-3.06 (m, 6H), 2.91-2.68 (m, 10H), 2.57-2.46 (m, 8H), 2.35 (s, 3H), 2.19-2.09 (m, 1H), 2.03-1.93 (m, 2H), 1.79-1.71 (m, 1H). [M+H]⁺=816.7.

### Example 20: (R)-3-(4-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(methoxymethyl)piperazin-1-yl)piperidin-1-yl)-2,6-difluorophenyl)piperidine-2,6-dione

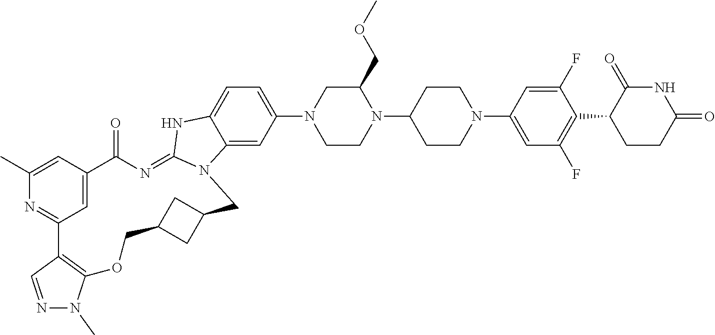 [SMILES: COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2cc(F)c([C@H]3CCC(=O)NC3=O)c(F)c2)CC1]

The titled compound was prepared in a manner similar to that in Example 6 with intermediate 6. ¹H NMR (500 MHz, DMSO) δ 12.41 (s, 1H), 10.87 (s, 1H), 8.65 (s, 1H), 7.89 (s, 1H), 7.46 (s, 1H), 7.35 (d, J=8.7 Hz, 1H), 6.97 (s, 1H), 6.86 (d, J=8.9 Hz, 1H), 6.64 (d, J=12.7 Hz, 2H), 4.82-4.13 (m, 4H), 4.07-4.03 (m, 2H), 3.83 (d, J=11.7 Hz, 2H), 3.72 (s, 3H), 3.57-3.56 (m, 1H), 3.50-3.48 (m, 1H), 3.28 (d, J=12.1 Hz, 5H), 3.21 (s, 1H), 3.02 (s, 2H), 2.98-2.86 (m, 4H), 2.83-2.70 (m, 5H), 2.65-2.64 (m, 2H), 2.55 (s, 4H), 2.1-2.08 (m, 1H), 1.96 (d, J=5.3 Hz, 1H), 1.89-1.87 (m, 1H), 1.72 (s, 1H), 1.62 (d, J=9.7 Hz, 1H), 1.45-1.43 (m, 1H); [M+H]⁺=863.7.

### Example 21: 3-(5-(4-(4-((7¹s,7³s,E)-1¹,2⁶-dimethyl-3-oxo-5²,5³-dihydro-1H,5¹H-9-oxa-4-aza-5(2,1)- benzo[d]imidazola-2(2,4)-pyridina-1(4,5)-pyrazola-7(1,3)-cyclobutanacyclononaphane-5⁶-yl)piperazin-1-yl)piperidin-1-yl)-6-methylpyridin-2-yl)piperidine-2,6-dione

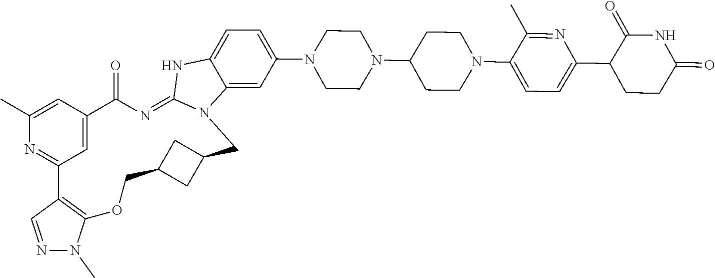 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC4)cc23)C1]

To a solution of (7¹s,7³s,E)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (50 mg, 0.1 mmol), and intermediate 12 (35 mg, 0.12 mmol) in DCE (10 mL) was added STAB (41 mg, 0.2 mmol). Then the mixture was stirred at 50° C. for 6 hr. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=20:1) to afford the product (11.1 mg, 14.3%). ¹H NMR (500 MHz, DMSO) δ 12.41 (s, 1H), 10.78 (s, 1H), 8.66 (s, 1H), 7.89 (s, 1H), 7.46 (s, 1H), 7.36 (dd, J=12.4, 8.5 Hz, 2H), 7.11 (d, J=8.1 Hz, 1H), 7.02 (s, 1H), 6.91 (d, J=10.8 Hz, 1H), 4.98-3.94 (m, 4H), 3.91-3.88 (m, 1H), 3.72 (s, 3H), 3.29 (s, 1H), 3.18-3.13 (m, 6H), 2.92 (s, 2H), 2.72 (s, 4H), 2.67-2.62 (m, 3H), 2.60-2.52 (m, 5H), 2.40-2.36 (m, 5H), 2.24-2.16 (m, 2H), 2.10-2.06 (m, 1H), 1.95-1.92 (m, 2H), 1.64-1.62 (m, 2H); [M+H]⁺=798.7.

### Example 22: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-oxopiperazin-1-yl)piperidin-1-yl)-6-methylpyridin-2-yl)piperidine-2,6-dione

### Step 1: tert-butyl 4-(3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)-4-nitrophenyl)-3-oxopiperazine-1-carboxylate

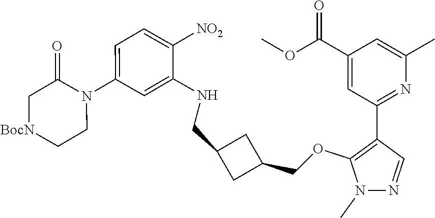 [SMILES: COC(=O)c1cc(C)nc(-c2cnn(C)c2OC[C@H]2C[C@@H](CNc3cc(N4CCN(C(=O)OC(C)(C)C)CC4=O)ccc3[N+](=O)[O-])C2)c1]

To a stirred solution of methyl 2-(5-(((1s,3s)-3-(((5-bromo-2-nitrophenyl)amino)methyl)cyclobutyl)methoxy)-1-methyl-1H-pyrazol-4-yl)-6-methylisonicotinate (1.0 g, 1.83 mmol) and tert-butyl 3-oxopiperazine-1-carboxylate (1.1 g, 5.49 mmol) in DMSO (20 mL) were added N1,N2-dimethylethane-1,2-diamine (80 mg, 0.92 mmol), CuI (175 mg, 0.92 mmol) and K₃PO₄ (1.36 g, 6.4 mmol). The resulting mixture was stirred for 2 h at 100° C. under nitrogen atmosphere. The reaction solution was diluted with water, extracted with EtOAc (50 mL×2). The combined organic layers were washed with water and brine, dried over anhydrous Na₂SO₄, filtered and concentrated under reduced pressure. The residue was purified by column chromatography (DCM:MeOH=100/0 to 100/4) to afford the product (520 mg, 43.3% yield). [M+H]⁺=664.5.

### Step 2: tert-butyl 4-(4-amino-3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)phenyl)-3-oxopiperazine-1-carboxylate

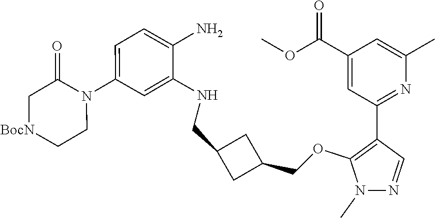 [SMILES: COC(=O)c1cc(C)nc(-c2cnn(C)c2OC[C@H]2C[C@@H](CNc3cc(N4CCN(C(=O)OC(C)(C)C)CC4=O)ccc3N)C2)c1]

A mixture of tert-butyl 4-(3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)-4-nitrophenyl)-3-oxopiperazine-1-carboxylate (500 mg, 0.75 mmol) and Raney-Ni (500 mg) in THF (10 mL) was degassed and purged with H₂ (40 Psi) for 3 times at room temperature, and then the mixture was stirred for 1.5 hours under H₂ atmosphere and turned to black suspension. The mixture was filtered and concentrated under reduced pressure. The product (410 mg, 86.3% yield) was used for next step without purification. [M+H]⁺=634.5.

### Step 3: tert-butyl 4-(2-amino-1-(((1s,3s)-3-(((4-(4-(methoxycarbonyl)-6-methylpyridin-2-yl)-1-methyl-1H-pyrazol-5-yl)oxy)methyl)cyclobutyl)methyl)-1H-benzo[d]imidazol-6-yl)-3-oxopiperazine-1-carboxylate

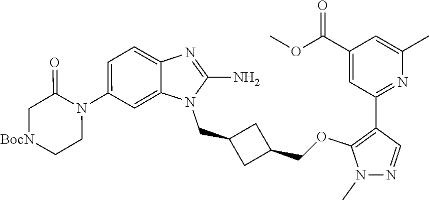 [SMILES: COC(=O)c1cc(C)nc(-c2cnn(C)c2OC[C@H]2C[C@@H](Cn3c(N)nc4ccc(N5CCN(C(=O)OC(C)(C)C)CC5=O)cc43)C2)c1]

To a solution of tert-butyl 4-(4-amino-3-((((1s,3s)-3-(((4-(4-(methoxycarbonyl)-6-methylpyridin-2-yl)-1-methyl-1H-pyrazol-5-yl)oxy)methyl)cyclobutyl)methyl)amino)phenyl)-3-oxopiperazine-1-carboxylate (410 mg, 0.64 mmol) in DCM (8 mL) and t-BuOH (2 mL) was added CNBr (203 mg, 1.92 mmol) at 25° C. The mixture was stirred at 25° C. for 16 hours. Then the reaction mixture was quenched by sat. aq. NaHCO₃ (10 mL). The layers were separated and the organic layer was washed with sat. aq. NaHCO₃ (10 mL×2). The organic layer was dried over Na₂SO₄, filtered and concentrated under reduced pressure to give the product (270 mg, 64.1% yield) which was used in the next step without further purification. [M+H]⁺=659.7

### Step 4: 2-(5-(((1s,3s)-3-((2-amino-6-(4-(tert-butoxycarbonyl)-2-oxopiperazin-1-yl)-1H-benzo[d]imidazol-1-yl)methyl)cyclobutyl)methoxy)-1-methyl-1H-pyrazol-4-yl)-6-methylisonicotinic acid

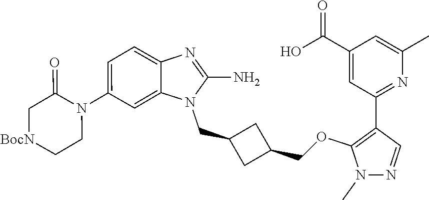 [SMILES: Cc1cc(C(=O)O)cc(-c2cnn(C)c2OC[C@H]2C[C@@H](Cn3c(N)nc4ccc(N5CCN(C(=O)OC(C)(C)C)CC5=O)cc43)C2)n1]

To a solution of tert-butyl 4-(2-amino-1-(((1s,3s)-3-(((4-(4-(methoxycarbonyl)-6-methylpyridin-2-yl)-1-methyl-1H-pyrazol-5-yl)oxy)methyl)cyclobutyl)methyl)-1H-benzo[d]imidazol-6-yl)-3-oxopiperazine-1-carboxylate (270 mg, 0.41 mmol) in THF (8 mL) was added NaOH (32.8 mg, 0.82 mmol) in H₂O (20 mL) at 25° C. The mixture was stirred at 25° C. for 2 hours. Then the reaction mixture was concentrated under reduced pressure to remove solvent. The residue was acidified with 4M aqueous HCl to pH=6, resulting in the formation of a slurry. The solid was filtered, washed with water (5 mL) and dried under reduced pressure to give the product. The product (220 mg, crude) was used for next step without purification. [M+H]⁺=645.7.

### Step 5: tert-butyl 4-((7¹s,7³s,E)-1¹,2⁶-dimethyl-3-oxo-5²,5³-dihydro-1′H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-oxopiperazine-1-carboxylate

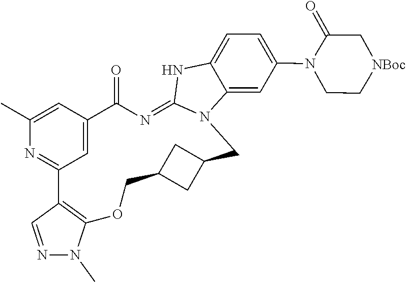 [SMILES: Cc1cc2cc(n1)-c1cnn(C)c1OC[C@H]1C[C@@H](CN3/C(=N/C2=O)Nc2ccc(N4CCN(C(=O)OC(C)(C)C)CC4=O)cc23)C1]

To a solution of 2-(5-(((1s,3s)-3-((2-amino-6-(4-(tert-butoxycarbonyl)-2-oxopiperazin-1-yl)-1H-benzo[d]imidazol-1-yl)methyl)cyclobutyl)methoxy)-1-methyl-1H-pyrazol-4-yl)-6-methylisonicotinic acid (220 mg, 0.34 mmol) in DCM (10 mL) was added HATU (156 mg, 0.41 mmol) and DIEA (132 mg, 1.02 mmol) at 25° C. The mixture was stirred at 25° C. for 1 hour and turned to a black solution. The reaction mixture was washed with sat. aq. NaHCO₃ (10 mL×3), dried over Na₂SO₄, filtered and concentrated under reduced pressure to give a residue. The residue was purified by column chromatography (DCM:MeOH=100/1 to 10/1) to afford the product (110 mg, 51.6% yield). [M+H]⁺=627.7.

### Step 6: (7¹s,7³s,E)-1¹,2⁶-dimethyl-5⁶-(2-oxopiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

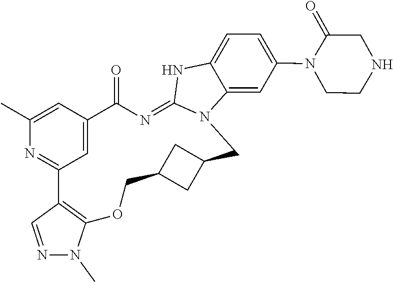 [SMILES: Cc1cc2cc(n1)-c1cnn(C)c1OC[C@H]1C[C@@H](CN3/C(=N/C2=O)Nc2ccc(N4CCNCC4=O)cc23)C1]

To a solution of tert-butyl 4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-oxopiperazine-1-carboxylate (110 mg, 0.175 mmol) in DCM (6 mL) was added TFA (2 mL). The reaction solution was stirred at r.t for 1.5 hrs and then concentrated in vacuo. The residue was dissolved in DCM and then washed with sat. aq. NaHCO₃ (5 mL) and brine (5 mL), dried over Na₂SO₄, filtered and concentrated in vacuum to afford the product (85 mg, 92.4%). [M+H]⁺=527.7.

### Step 7: 3-(5-(4-(4-((7¹s,7³s,E)-1¹,2⁶-dimethyl-3-oxo-5²,5³-dihydro-1¹H,5¹H-9-oxa-4-aza-5(2,1)- benzo[d]imidazola-2(2,4)-pyridina-1(4,5)-pyrazola-7(1,3)-cyclobutanacyclononaphane-5⁶-yl)-3-oxopiperazin-1-yl)piperidin-1-yl)-6-methylpyridin-2-yl)piperidine-2,6-dione

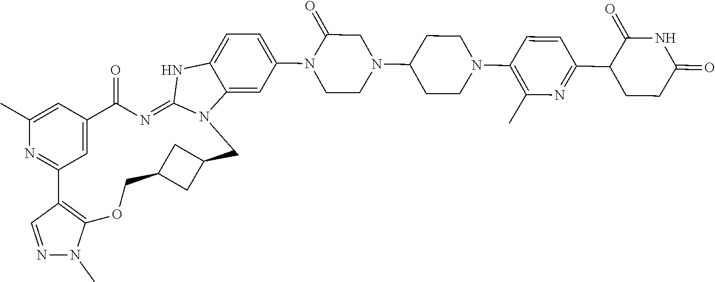 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC4=O)cc23)C1]

The titled compound was prepared in a manner similar to that in Example 21 with intermediate 12.

¹H NMR (500 MHz, DMSO) δ 12.67 (s, 1H), 10.78 (s, 1H), 8.67 (s, 1H), 7.90 (s, 1H), 7.49 (d, J=4.1 Hz, 2H), 7.38 (d, J=8.2 Hz, 1H), 7.18 (d, J=9.9 Hz, 1H), 7.11 (d, J=8.1 Hz, 2H), 4.19-4.13 (m, 2H), 3.91-3.88 (m, 1H), 3.76-3.71 (m, 4H), 3.69-3.66 (m, 2H), 3.36 (s, 3H), 3.18-3.12 (m, 2H), 2.99-2.89 (m, 5H), 2.68-2.62 (m, 3H), 2.60-2.58 (m, 1H), 2.56 (s, 3H), 2.44-2.40 (m, 3H), 2.27-2.20 (m, 2H), 2.13-2.06 (m, 2H), 1.97-1.93 (m, 3H), 1.67-1.61 (m, 3H); [M+H]⁺=812.7.

### Example 23: 3-(5-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(methoxymethyl)piperazin-1-yl)piperidin-1-yl)-4-methylpyridin-2-yl)piperidine-2,6-dione

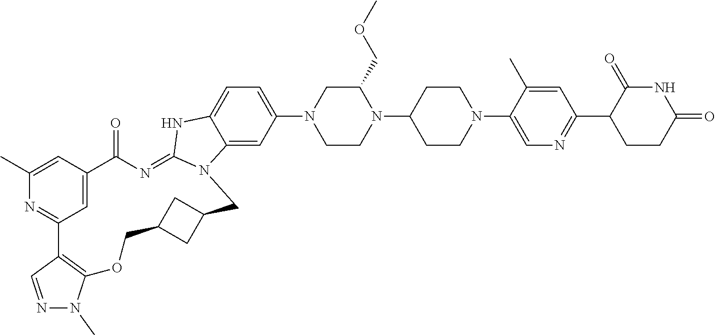 [SMILES: COC[C@H]1CN(c2ccc3c(c2)N2C[C@H]4C[C@@H](COc5c(cnn5C)-c5cc(cc(C)n5)C(=O)/N=C/2N3)C4)CCN1C1CCN(c2cnc(C3CCC(=O)NC3=O)cc2C)CC1]

The titled compound was prepared in a manner similar to that in Example 30 with intermediate 10. ¹H NMR (500 MHz, DMSO) δ 12.35 (s, 1H), 10.73 (s, 1H), 8.59 (s, 1H), 8.08 (s, 1H), 7.82 (s, 1H), 7.39 (s, 1H), 7.29 (d, J=8.7 Hz, 1H), 7.07 (s, 1H), 6.92 (s, 1H), 6.81 (d, J=8.7 Hz, 1H), 4.87-3.91 (m, 4H), 3.86-3.80 (m, 1H), 3.65 (s, 3H), 3.54-3.46 (m, 1H), 3.45-3.40 (m, 1H), 3.23-3.07 (m, 6H), 3.03-2.93 (m, 2H), 2.92-2.78 (m, 5H), 2.75-2.68 (m, 1H), 2.67-2.61 (m, 2H), 2.55-2.45 (m, 10H), 2.19 (s, 3H), 2.18-2.10 (m, 1H), 2.09-2.00 (m, 1H), 1.90-1.84 (m, 1H), 1.75-1.67 (m, 2H), 1.54-1.46 (m, 1H). [M+H]⁺=842.7.

### Example 24: 3-(5-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(methoxymethyl)piperazin-1-yl)piperidin-1-yl)-6-methylpyridin-2-yl)piperidine-2,6-dione

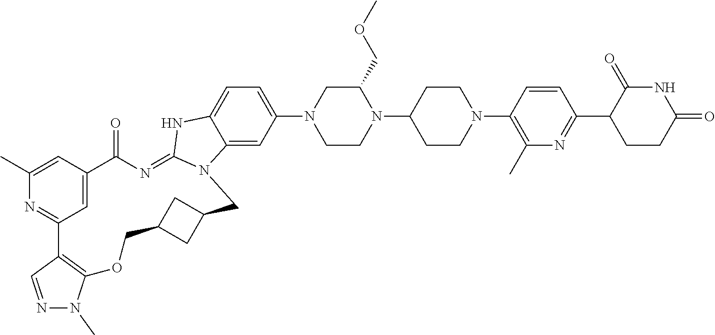 [SMILES: COC[C@H]1CN(c2ccc3c(c2)N2C[C@H]4C[C@@H](COc5c(cnn5C)-c5cc(cc(C)n5)C(=O)/N=C/2N3)C4)CCN1C1CCN(c2ccc(C3CCC(=O)NC3=O)nc2C)CC1]

The titled compound was prepared in a manner similar to that in Example 30 with intermediate 12.

¹H NMR (500 MHz, DMSO) δ 12.34 (s, 1H), 10.72 (s, 1H), 8.59 (s, 1H), 7.83 (s, 1H), 7.39 (s, 1H), 7.30 (t, J=8.5 Hz, 2H), 7.03 (d, J=8.1 Hz, 1H), 6.91 (s, 1H), 6.81 (d, J=8.7 Hz, 1H), 4.92-3.90 (m, 4H), 3.82 (dd, J=9.4, 5.3 Hz, 1H), 3.65 (s, 3H), 3.52-3.46 (m, 1H), 3.45-3.40 (m, 1H), 3.24-3.15 (m, 6H), 3.11-3.04 (m, 2H), 2.99-2.92 (m, 2H), 2.90-2.76 (m, 5H), 2.67-2.59 (m, 2H), 2.56-2.44 (m, 9H), 2.34 (s, 3H), 2.19-2.11 (m, 1H), 2.06-1.97 (m, 1H), 1.89-1.83 (m, 1H), 1.71 (s, 2H), 1.56-1.47 (m, 1H). [M+H]⁺=842.8.

Example 25: (R)-3-(4-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-oxopiperazin-1-yl)piperidin-1-yl)-2,6-difluorophenyl)piperine-2,6-dione

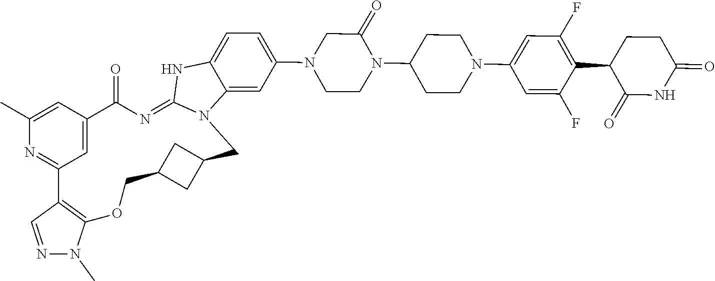 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)C(=O)C4)cc23)C1]

The titled compound was prepared in a manner similar to that in Example 26. ¹H NMR (500 MHz, DMSO) δ 12.43 (s, 1H), 10.88 (s, 1H), 8.66 (s, 1H), 7.89 (s, 1H), 7.46 (s, 1H), 7.37 (d, J=8.7 Hz, 1H), 7.07 (s, 1H), 6.90 (d, J=8.8 Hz, 1H), 6.68 (d, J=12.7 Hz, 2H), 4.84-4.44 (m, 3H), 4.34-4.01 (m, 3H), 3.94-3.83 (m, 4H), 3.72 (s, 3H), 3.49-3.36 (m, 2H), 3.42-3.39 (m, 2H), 3.29 (s, 1H), 2.99-2.75 (m, 6H), 2.58-2.52 (m, 7H), 2.13-2.05 (m, 1H), 1.97-1.96 (m, 1H), 1.80-1.78 (m, 2H), 1.64-1.62 (m, 1H); [M+H]⁺=833.7.

### Example 26: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-oxopiperazin-1-yl)piperidin-1-yl)-6-methylpyridin-2-yl)piperidine-2,6-dione

### Step 1: tert-butyl 4-(1-(2′,6′-bis(benzyloxy)-6-methyl-[2,3′-bipyridin]-5-yl)piperidin-4-yl)-3-oxopiperazine-1-carboxylate

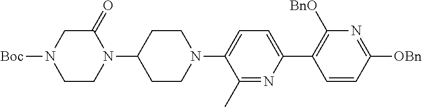 [SMILES: Cc1nc(-c2ccc(OCc3ccccc3)nc2OCc2ccccc2)ccc1N1CCC(N2CCN(C(=O)OC(C)(C)C)CC2=O)CC1]

To a stirred solution of 2′,6′-bis(benzyloxy)-5-bromo-6-methyl-2,3′-bipyridine (1.5 g, 3.25 mmol) (the compound was obtained through the similar method of intermediate 10 step 1) and tert-butyl 3-oxo-4-(piperidin-4-yl)piperazine-1-carboxylate (1.2 g, 4.23 mmol) in DMA (30 mL) were added Cs₂CO₃ (3.17 g, 9.75 mmol), Pd₂(dba)₃ (300 mg, 0.325 mmol) and Ruphos (300 mg, 0.65 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (200 mL), washed with water (3×100 mL) and brine (100 mL). The organic layer was dried over anhydrous Na₂SO₄, filtered and concentrated under reduced pressure. The residue was purified by silica gel column chromatography, eluted with PE/EtOAc (4:1) to afford the product (350 mg, 16.7%); [M+H]⁺=664.7.

### Step 2: tert-butyl 4-(1-(6-(2,6-dioxopiperidin-3-yl)-2-methylpyridin-3-yl)piperidin-4-yl)-3-oxopiperazine-1-carboxylate

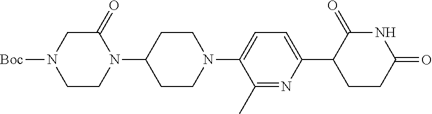 [SMILES: Cc1nc(C2CCC(=O)NC2=O)ccc1N1CCC(N2CCN(C(=O)OC(C)(C)C)CC2=O)CC1]

tert-butyl 4-(1-(2′,6′-bis(benzyloxy)-6-methyl-[2,3′-bipyridin]-5-yl)piperidin-4-yl)-3-oxopiperazine-1-carboxylate (350 mg, 0.53 mmol) was dissolved in DMF (8 mL) and iPrOH (4 mL). Pd/C (350 mg, 10 wt. %, wet) was added to the solution in one portion. The resulting mixture was stirred under hydrogen atmosphere (1 atm) at 50° C. overnight. The solid was filtered off and the filtrate was concentrated to give the crude product. The crude was purified by silica gel column chromatography, eluted with DMC/MeOH (20:1) to afford the product (190 mg, 73.9%); [M+H]⁺=486.5.

### Step 3: 3-(6-methyl-5-(4-(2-oxopiperazin-1-yl)piperidin-1-yl)pyridin-2-yl)piperidine-2,6-dione

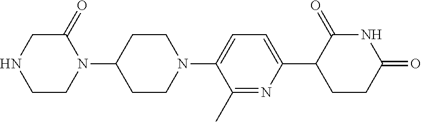 [SMILES: Cc1nc(C2CCC(=O)NC2=O)ccc1N1CCC(N2CCNCC2=O)CC1]

To a 100-mL round-bottomed flask equipped with a magnetic stir bar was added tert-butyl 4-(1-(6-(2,6-dioxopiperidin-3-yl)-2-methylpyridin-3-yl)piperidin-4-yl)-3-oxopiperazine-1-carboxylate (190 mg, 0.39 mmol), DCM (6 mL) and TFA (2 mL). After stirring for 1 h at room temperature, the reaction mixture was concentrated under reduced pressure to afford the product as TFA salt (260 mg), which was used without further purification. [M+H]⁺=386.4.

### Step 4: 3-(5-(4-(4-((7¹s,7³s,E)-1¹,2⁶-dimethyl-3-oxo-5²,5³-dihydro-1¹H,5¹H-9-oxa-4-aza-5(2,1)- benzo[d]imidazola-2(2,4)-pyridina-1(4,5)-pyrazola-7(1,3)-cyclobutanacyclononaphane-5⁶-yl)-2-oxopiperazin-1-yl)piperidin-1-yl)-6-methylpyridin-2-yl)piperidine-2,6-dione

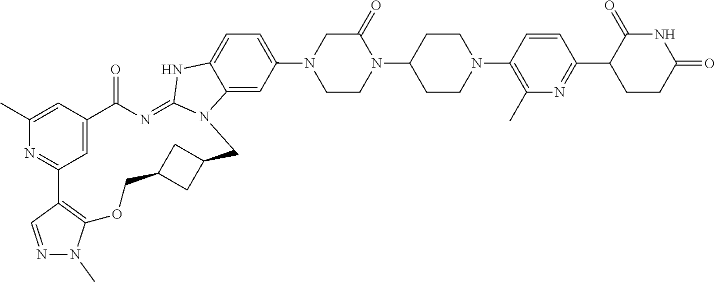 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C(=O)C4)cc23)C1]

To a stirred solution of intermediate 1 (150 mg, 0.29 mmol) and 3-(6-methyl-5-(4-(2-oxopiperazin-1-yl)piperidin-1-yl)pyridin-2-yl)piperidine-2,6-dione (TFA salt) (209 mg, 0.44 mmol) in DMA (10 mL) were added t-BuONa (141 mg, 1.47 mmol), Ruphos (55 mg, 0.12 mmol) and Pd₂(dba)₃ (54 mg, 0.06 mmol) at room temperature under nitrogen atmosphere. The resulting mixture was stirred for 2 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with DCM/MeOH (10:1) to afford the crude product, followed by Prep-HPLC chromatography to afford the title product (6.12 mg, 2.6%). ¹H NMR (500 MHz, DMSO) δ 12.43 (s, 1H), 10.79 (s, 1H), 8.66 (s, 1H), 7.89 (s, 1H), 7.46 (s, 1H), 7.42 (d, J=8.2 Hz, 1H), 7.38 (d, J=8.6 Hz, 1H), 7.12 (d, J=8.1 Hz, 1H), 7.08 (s, 1H), 6.92 (d, J=8.8 Hz, 1H), 4.49-4.41 (m, 1H), 3.93-3.88 (m, 2H), 3.87 (s, 2H), 3.72 (s, 4H), 3.55-3.46 (m, 6H), 3.16 (d, J=9.6 Hz, 2H), 2.98-2.92 (m, 3H), 2.76 (t, J=11.4 Hz, 3H), 2.59-2.51 (m, 7H), 2.43 (s, 3H), 2.29-2.21 (m, 1H), 2.11-2.07 (m, 1H), 1.97-1.92 (m, 2H), 1.72-1.65 (m, 2H); [M+H]⁺=812.7.

### Example 27: (R)-3-(4-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(methoxymethyl)piperazin-1-yl)piperidin-1-yl)-2,6-difluorophenyl)piperidine-2,6-dione

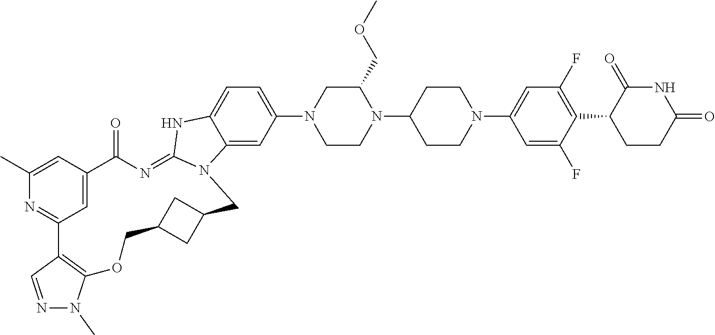 [SMILES: COC[C@H]1CN(c2ccc3c(c2)N2C[C@H]4C[C@@H](COc5c(cnn5C)-c5cc(cc(C)n5)C(=O)/N=C/2N3)C4)CCN1C1CCN(c2cc(F)c([C@H]3CCC(=O)NC3=O)c(F)c2)CC1]

The titled compound was prepared in a manner similar to that in Example 6 with intermediate 6.

¹H NMR (500 MHz, DMSO) δ 12.40 (s, 1H), 10.87 (s, 1H), 8.65 (s, 1H), 7.89 (s, 1H), 7.46 (s, 1H), 7.35 (d, J=8.7 Hz, 1H), 6.97 (s, 1H), 6.86 (d, J=7.4 Hz, 1H), 6.64 (d, J=12.8 Hz, 2H), 4.96-4.08 (m, 4H), 4.08-4.02 (m, 1H), 3.83 (d, J=12.4 Hz, 2H), 3.72 (s, 3H), 3.58-3.56 (m, 1H), 3.51-3.48 (m, 1H), 3.28 (d, J=11.8 Hz, 4H), 3.20 (s, 1H), 3.02 (s, 2H), 2.99-2.85 (m, 5H), 2.82-2.64 (m, 6H), 2.55-2.52 (m, 6H), 2.15-2.04 (m, 1H), 1.96 (d, J=5.1 Hz, 1H), 1.89-1.86 (m, 1H), 1.74-1.72 (m, 1H), 1.76-1.59 (m, 1H), 1.47-1.40 (m, 1H); [M+H]⁺=863.7.

Example 28: 3-(5-(4-(1-((7¹s,7³s,E)-1¹,2⁶-dimethyl-3-oxo-5²,5³-dihydro-1¹H,5¹H-9-oxa-4-aza-5(2,1)- benzo[d]imidazola-2(2,4)-pyridina-1(4,5)-pyrazola-7(1,3)-cyclobutanacyclononaphane-5⁶-yl)piperidin-4-yl)piperazin-1-yl)-6-methylpyridin-2-yl)piperidine-2,6-dione

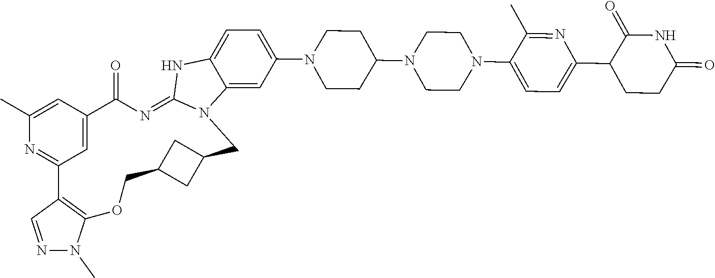 [SMILES: Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)CC5)CC4)cc23)C1]

To a solution of (7¹s,7³s,E)-1¹,2⁶-dimethyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (80 mg, 0.15 mmol), intermediate 15 (60 mg, 0.18 mmol) and DIEA (58 mg, 0.45 mmol) in DCE (8 mL) was added STAB (95 mg, 0.45 mmol). Then the mixture was stirred at 50° C. for 16 hours. H₂O (15 mL) was added and the resulting mixture was extracted with DCM (15 mL×3). The combined organic phase was washed with brine (10 mL), dried over Na₂SO₄, filtered and concentrated in vacuum. The residue was purified by silica gel column (DCM:CH₃OH=10:1), followed by prep-HPLC chromatography to afford the title product (35 mg, 28.9%). ¹H NMR (500 MHz, DMSO) δ 12.31 (s, 1H), 10.78 (s, 1H), 8.65 (s, 1H), 7.89 (s, 1H), 7.46 (s, 1H), 7.40-7.31 (m, 2H), 7.11 (d, J=8.1 Hz, 1H), 7.02 (s, 1H), 6.91 (d, J=8.9 Hz, 1H), 4.85-4.03 (m, 4H), 3.89 (dd, J=9.4, 5.3 Hz, 1H), 3.77-3.70 (m, 5H), 3.61-3.50 (m, 1H), 3.00-2.81 (m, 7H), 2.75-2.63 (m, 6H), 2.62-2.52 (m, 6H), 2.45-2.35 (m, 4H), 2.28-2.15 (m, 2H), 2.08 (dd, J=13.3, 5.5 Hz, 1H), 1.92 (d, J=11.3 Hz, 2H), 1.65-1.55 (m, 2H). [M+H]⁺=798.6.

### Example 29: (R)-3-(4-(3-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)azetidin-1-yl)-2,6-difluorophenyl)piperidine-2,6-dione

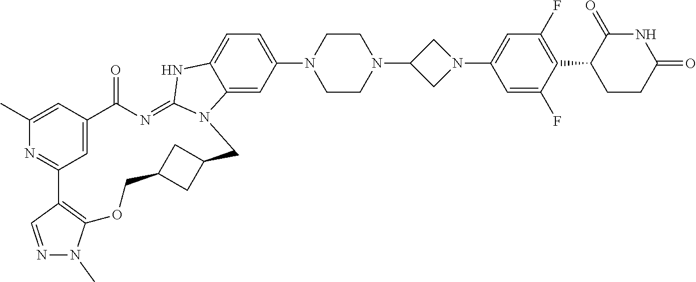 [SMILES: Cc1cc2cc(n1)-c1cnn(C)c1OC[C@H]1C[C@@H](CN3/C(=N/C2=O)Nc2ccc(N4CCN(C5CN(c6cc(F)c([C@H]7CCC(=O)NC7=O)c(F)c6)C5)CC4)cc23)C1]

The titled compound was prepared in a manner similar to that in Example 6 with intermediate 7.

¹H NMR (500 MHz, DMSO) δ 12.34 (s, 1H), 10.79 (s, 1H), 8.59 (s, 1H), 7.82 (s, 1H), 7.39 (s, 1H), 7.28 (d, J=8.7 Hz, 1H), 6.96 (s, 11H), 6.84 (d, J=8.9 Hz, 11H), 6.08 (d, J=11.2 Hz, 2H), 4.81-4.38 (m, 3H), 4.24-4.07 (m, 1H), 4.04-3.94 (m, 2H), 3.91-3.82 (m, 2H), 3.73-3.58 (m, 5H), 3.20-3.06 (m, 8H), 2.91-2.79 (m, 2H), 2.75-2.67 (m, 1H), 2.63-2.47 (m, 6H), 2.28-1.96 (m, 2H), 1.94-1.43 (m, 2H). [M+H]⁺=791.5.

### Example 30: (R)-3-(4-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(hydroxymethyl)piperazin-1-yl)piperidin-1-yl)-2,6-difluorophenyl)piperidine-2,6-dione

### Step 1: tert-butyl (S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(hydroxymethyl)piperazine-1-carboxylate

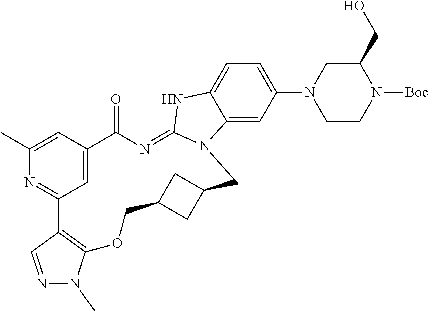 [SMILES: Cc1cc2cc(n1)-c1cnn(C)c1OC[C@H]1C[C@@H](CN3/C(=N/C2=O)Nc2ccc(N4CCN(C(=O)OC(C)(C)C)[C@H](CO)C4)cc23)C1]

A mixture of intermediate 1 (506 mg, 1 mmol), tert-butyl (R)-2-(hydroxymethyl)piperazine-1-carboxylate (325 mg, 1.5 mmol), Pd₂dba₃(91 mg, 0.1 mmol), Ruphos (93 mg, 0.2 mmol), and t-BuONa (288 mg, 3 mmol) in DMA (10 mL) was stirred in a round bottom flask at 90° C. for 2 hours under N₂. Water (20 mL) was added, and the mixture was extracted with DCM (50 mL×3). The combined organic layer was dried over Na₂SO₄. The solvent was removed by evaporation, and the residue was purified by silica gel column chromatography (DCM:MeOH=100:0˜80:20 gradient elution) to afford the product (420 mg, 66%). [M+H]⁺=643.2.

### Step 2: (7¹s,7³R,E)-5⁶-((S)-3-(hydroxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

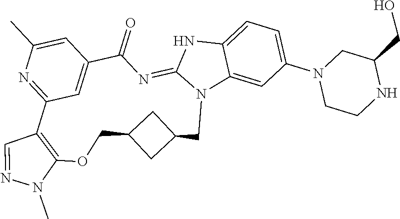 [SMILES: Cc1cc2cc(n1)-c1cnn(C)c1OC[C@H]1C[C@@H](CN3/C(=N/C2=O)Nc2ccc(N4CCN[C@H](CO)C4)cc23)C1]

To a solution of tert-butyl (S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(hydroxymethyl)piperazine-1-carboxylate (129 mg, 0.2 mmol) in DCM (4 mL) was added TFA (1 mL) at 25° C. The mixture was stirred at rt for 1 hour. Sat. sq. NaHCO₃ (20 mL) was added, and the mixture was extracted with DCM (50 mL×3). The combined organic layer was dried over Na₂SO₄. The solvent was concentrated under reduced pressure to give the product (90 mg, 85%). [M+H]⁺=543.2.

### Step 3: (R)-3-(4-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(hydroxymethyl)piperazin-1-yl)piperidin-1-yl)-2,6-difluorophenyl)piperidine-2,6-dione

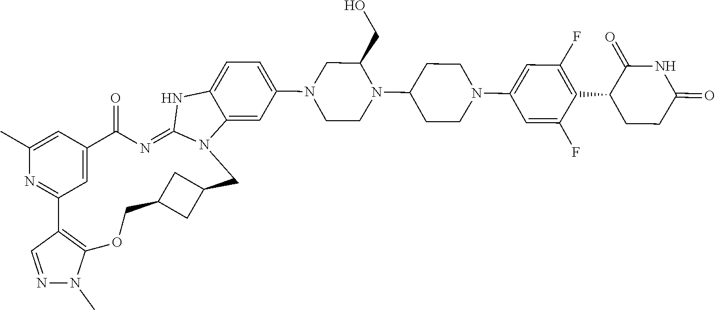 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)[C@H](CO)C4)cc23)C1]

A mixture of (7¹s,7³R,E)-5⁶-((S)-3-(hydroxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (100 mg, 0.18 mmol) and intermediate 6 (65 mg, 0.2 mmol) in 1,2-dichloroethane (15 mL)) was stirred in a round bottom flask at 70° C. for 2 hours. To the mixture was added NaBH(OAc)₃ (80 mg, 0.37 mmol) and the reaction was stirred at 70° C. for 12 hours. Then the mixture was evaporated in vacuum to afford the crude product, which was purified with silica gel column chromatography (DCM:MeOH=100:0˜80:20 gradient elution) to give the product (15 mg, 10%). ¹H NMR (500 MHz, DMSO) δ 12.42 (s, 1H), 10.88 (s, 1H), 8.66 (s, 1H), 7.90 (s, 1H), 7.46 (s, 1H), 7.36 (d, J=8.6 Hz, 1H), 6.98 (s, 1H), 6.90 (s, 1H), 6.66 (d, J=12.0 Hz, 2H), 4.63-4.61 (m, 2H), 4.32-4.15 (m, 1H), 4.05-4.01 (m, 2H), 3.85-3.81 (m, 2H), 3.72-3.69 (m, 3H), 3.70-3.65 (m, 1H), 3.30-3.18 (m, 9H), 3.10-2.98 (m, 3H), 2.92-2.88 (m, 3H), 2.81-2.76 (m, 4H), 2.56-2.52 (m, 4H), 2.10-1.95 (m, 1H), 1.94-1.89 (m, 2H), 1.77-1.72 (m, 2H), 1.46-1.42 (m, 1H); [M+H]⁺=848.9.

### Example 31: (R)-3-(4-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(hydroxymethyl)piperazin-1-yl)piperidin-1-yl)-2,6-difluorophenyl)piperidine-2,6-dione

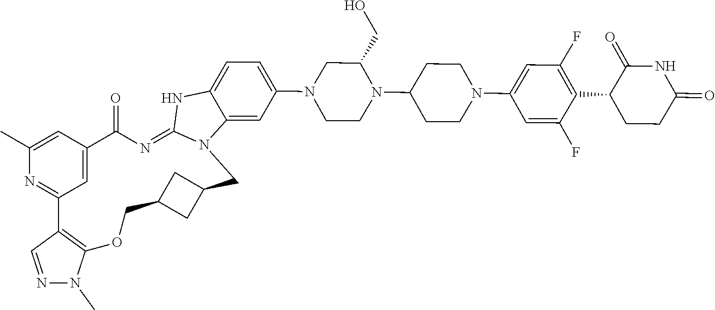 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)[C@@H](CO)C4)cc23)C1]

The titled compound was prepared in a manner similar to that in Example 30. ¹H NMR (500 MHz, DMSO) δ 12.41 (s, 1H), 10.87 (s, 1H), 8.65 (s, 1H), 7.91 (s, 1H), 7.45 (s, 1H), 7.36 (d, J=8.6 Hz, 1H), 6.98 (s, 1H), 6.90 (s, 1H), 6.66 (d, J=12.0 Hz, 2H), 4.63-4.61 (m, 2H), 4.32-4.15 (m, 1H), 4.05-4.01 (m, 2H), 3.85-3.81 (m, 2H), 3.72-3.69 (m, 3H), 3.70-3.65 (m, 1H), 3.30-3.18 (m, 9H), 3.10-2.98 (m, 3H), 2.92-2.88 (m, 3H), 2.81-2.76 (m, 4H), 2.56-2.52 (m, 4H), 2.10-1.95 (m, 1H), 1.94-1.90 (m, 2H), 1.77-1.73 (m, 2H), 1.46-1.41 (m, 1H); [M+H]⁺=848.9.

### Example 32: 3-(5-(4-((S)-4-((7¹s,7³R,E)-1¹,2⁶-dimethyl-3-oxo-5¹,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)-4-methylpyridin-2-yl)piperidine-2,6-dione

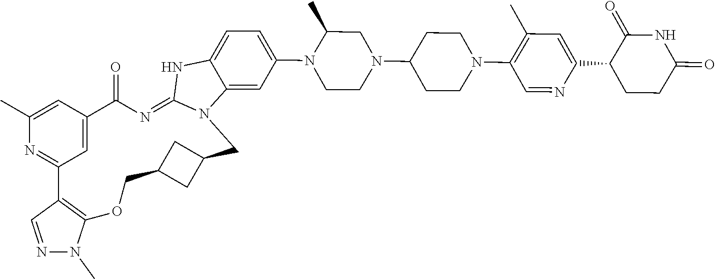 [SMILES: Cc1cc2cc(n1)-c1cnn(C)c1OC[C@H]1C[C@@H](CN3/C(=N/C2=O)Nc2ccc(N4CCN(C5CCN(c6cnc([C@H]7CCC(=O)NC7=O)cc6C)CC5)C[C@@H]4C)cc23)C1]

The titled compound was prepared in a manner similar to that in Example 33.

¹H NMR (500 MHz, DMSO) δ 12.40 (s, 1H), 10.79 (s, 1H), 8.65 (s, 1H), 8.14 (s, 1H), 7.88 (s, 1H), 7.46 (s, 1H), 7.36 (s, 1H), 7.13 (s, 1H), 7.01 (s, 1H), 6.87 (s, 1H), 4.83-4.07 (m, 4H), 3.95-3.83 (m, 2H), 3.70 (s, 3H), 3.28-3.13 (m, 6H), 3.06-2.86 (m, 6H), 2.79-2.60 (m, 7H), 2.43-2.32 (m, 2H), 2.29-2.03 (m, 6H), 1.95-1.85 (m, 2H), 1.69-1.52 (m, 2H), 1.05-0.90 (m, 3H). [M+H]⁺=812.4.

### Example 33: 3-(5-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)-6-methylpyridin-2-yl)piperidine-2,6-dione

### Step 1: tert-butyl (S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazine-1-carboxylate

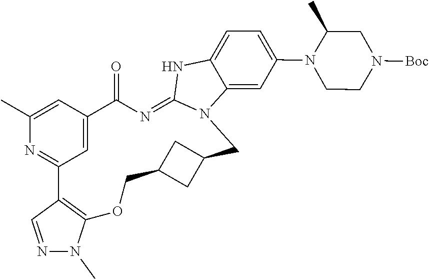 [SMILES: Cc1cc2cc(n1)-c1cnn(C)c1OC[C@H]1C[C@@H](CN3/C(=N/C2=O)Nc2ccc(N4CCN(C(=O)OC(C)(C)C)C[C@@H]4C)cc23)C1]

To a stirred solution of intermediate 1 (300 mg, 0.59 mmol) and tert-butyl (S)-3-methylpiperazine-1-carboxylate (237 mg, 1.18 mmol) in DMA (15 mL) were added t-BuONa (227 mg, 2.37 mmol), Ruphos (110 mg, 0.24 mmol) and Pd₂(dba)₃ (108 mg, 0.12 mmol) at room temperature under nitrogen atmosphere. The resulting mixture was stirred for 2 h at 100 T under nitrogen atmosphere. The mixture was allowed to cool down to room temperature. The resulting mixture was concentrated under reduced pressure. The residue was diluted with EtOAc (100 mL), and washed with water (3×50 mL) and brine (50 mL). The organic layer was dried over anhydrous Na₂SO₄, filtered and concentrated under reduced pressure. The residue was purified by silica gel column chromatography, eluted with DCM/MeOH (15:1) to afford the product (130 mg, 35.1%); [M+H]⁺=627.7.

### Step 2: (7¹s,7³R,E)-1¹,2⁶-dimethyl-5⁶-((S)-2-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one

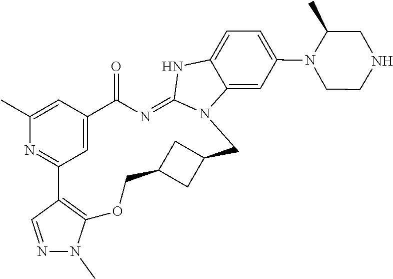 [SMILES: Cc1cc2cc(n1)-c1cnn(C)c1OC[C@H]1C[C@@H](CN3/C(=N/C2=O)Nc2ccc(N4CCNC[C@@H]4C)cc23)C1]

To a solution of tert-butyl (S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazine-1-carboxylate (130 mg, 0.21 mmol) in DCM (10 mL) was added TFA (5 mL). The reaction was stirred at rt for 2 hrs and then concentrated in vacuo. The residue was dissolved in DCM (30 mL), washed with sat. aq. NaHCO₃ (20 mL) and brine (20 mL), dried over Na₂SO₄, filtered and concentrated in vacuum to afford the product (70 mg, 64.10%). [M+H]⁺=527.6.

### Step 3: 3-(5-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)-6-methylpyridin-2-yl)piperidine-2,6-dione

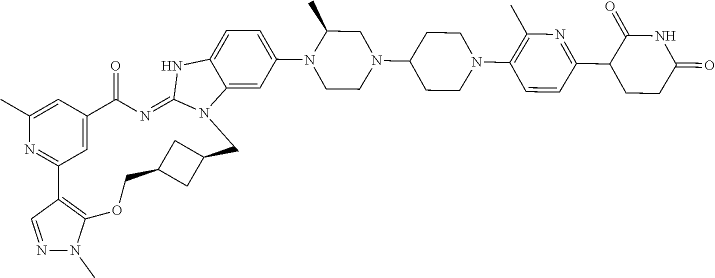 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C[C@@H]4C)cc23)C1]

To a solution of (7 s,7³R,E)-1¹,2⁶-dimethyl-5⁶-((S)-2-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphan-3-one (50 mg, 0.1 mmol), intermediate 12 (57 mg, 0.19 mmol) in DCE (5 mL) was added STAB (60 mg, 0.28 mmol). Then the mixture was stirred at 50° C. for 6 hr. H₂O (10 mL) was added and the resulting mixture was extracted with DCM (10 mL×3). The combined organic phase was washed with brine (10 mL), dried over Na₂SO₄, filtered and concentrated in vacuum. The residue was purified by silica gel column (DCM:CH₃OH=20:1) to afford the product (27.1 mg, 35.2%). ¹H NMR (500 MHz, DMSO) δ 12.34 (s, 1H), 10.72 (s, 1H), 8.59 (s, 1H), 7.82 (s, 1H), 7.40 (s, 1H), 7.30 (t, J=8.0 Hz, 2H), 7.04 (d, J=8.1 Hz, 1H), 6.95 (s, 1H), 6.83 (d, J=8.8 Hz, 1H), 4.83-3.94 (m, 4H), 3.97-3.81 (m, 2H), 3.65 (s, 3H), 3.17 (s, 1H), 3.09-3.07 (m, 2H), 2.95 (s, 1H), 2.84 (s, 3H), 2.65 (s, 1H), 2.62-2.54 (m, 4H), 2.53-2.45 (m, 10H), 2.34 (s, 3H), 2.15-2.12 (m, 1H), 2.03-1.99 (m, 1H), 1.84 (s, 2H), 1.57 (s, 2H), 0.92 (d, J=6.0 Hz, 3H); [M+H]⁺=812.6.

### Example 34: 3-(5-(4-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)-6-methylpyridin-2-yl)piperidine-2,6-dione

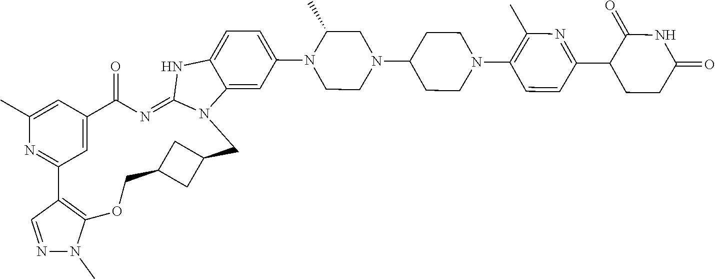 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C[C@H]4C)cc23)C1]

The titled compound was prepared in a manner similar to that in Example 33. ¹H NMR (500 MHz, DMSO) δ 12.34 (s, 1H), 10.71 (s, 1H), 8.59 (s, 1H), 7.82 (s, 1H), 7.39 (s, 1H), 7.30 (t, J=8.2 Hz, 2H), 7.04 (d, J=8.1 Hz, 1H), 6.95 (s, 1H), 6.82 (d, J=8.5 Hz, 1H), 4.80-3.95 (m, 4H), 3.87-3.81 (m, 2H), 3.65 (s, 3H), 3.16 (s, 1H), 3.08-3.07 (m, 2H), 2.94 (s, 1H), 2.84 (s, 3H), 2.65 (s, 1H), 2.60-2.57 (m, 4H), 2.53-2.44 (m, 10H), 2.34 (s, 3H), 2.18-2.09 (m, 1H), 2.03-1.99 (m, 1H), 1.84 (s, 2H), 1.58 (s, 2H), 0.92 (d, J=6.1 Hz, 3H); [M+H]⁺=812.6.

### Example 35: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-oxopiperazin-1-yl)piperidin-1-yl)pyridin-2-yl)piperidine-2,6-dione

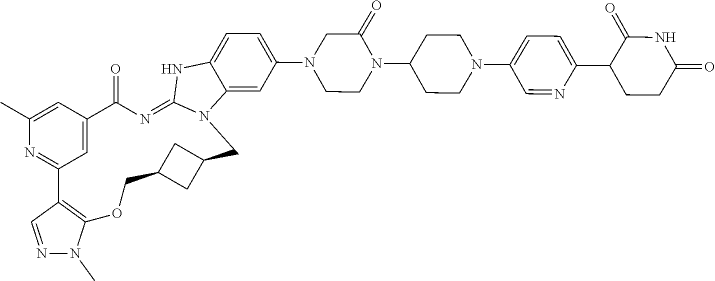 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C(=O)C4)cc23)C1]

The titled compound was prepared in a manner similar to that in Example 26. ¹H NMR (500 MHz, DMSO) δ 12.38 (s, 1H), 10.75 (s, 1H), 8.61 (s, 1H), 8.19 (s, 1H), 7.85 (s, 1H), 7.43 (s, 1H), 7.36 (s, 1H), 7.31 (d, J=8.6 Hz, 1H), 7.17 (s, 1H), 7.01 (s, 1H), 6.84 (d, J=9.4 Hz, 1H), 4.43-4.38 (m, 2H), 3.84-3.81 (m, 3H), 3.79-3.76 (m, 3H), 3.65 (s, 4H), 2.93-2.83 (m, 3H), 2.80-2.73 (m, 5H), 2.57 (s, 2H), 2.52-2.48 (m, 4H), 2.29 (s, 1H), 2.17-2.10 (m, 2H), 2.07-1.95 (m, 2H), 1.85-1.75 (m, 3H), 1.65-1.58 (m, 2H); [M+H]⁺=798.6 Example 36: 3-(5-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)piperidin-1-yl)pyridin-2-yl)piperidine-2,6-dione

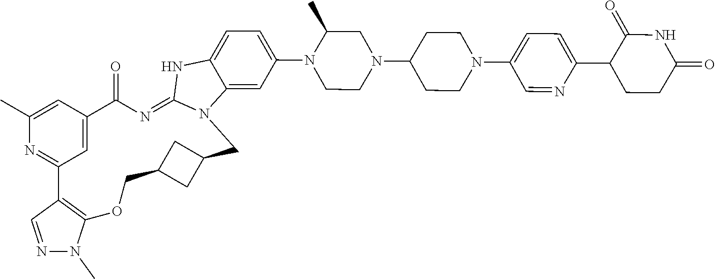 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C[C@@H]4C)cc23)C1]

The titled compound was prepared in a manner similar to that in Example 33 with intermediate 11.

¹H NMR (500 MHz, DMSO) δ 12.41 (s, 1H), 10.78 (s, 1H), 8.66 (s, 1H), 8.22 (d, J=2.6 Hz, 1H), 7.90 (s, 1H), 7.46 (s, 1H), 7.40-7.31 (m, 2H), 7.17 (d, J=8.7 Hz, 1H), 7.01 (s, 1H), 6.88 (d, J=8.6 Hz, 1H), 4.81-4.02 (m, 4H), 3.92-3.86 (m, 2H), 3.80-3.69 (m, 5H), 3.32-3.20 (m, 5H), 3.02-2.96 (m, 1H), 2.95-2.86 (m, 2H), 2.79-2.72 (m, 2H), 2.71-2.52 (m, 10H), 2.23-2.16 (m, 1H), 2.14-2.06 (m, 1H), 1.94-1.86 (m, 2H), 1.62-1.49 (m, 2H), 0.97 (d, J=6.2 Hz, 3H). [M+H]⁺=798.3.

### Example 37: 3-(5-(3-(((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)methyl)azetidin-1-yl)-4-methylpyridin-2-yl)piperidine-2,6-dione

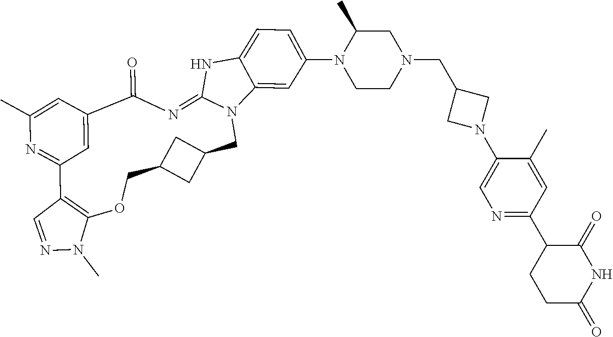 [SMILES: Cc1cc2cc(n1)-c1cnn(C)c1OC[C@H]1C[C@@H](CN3/C(=N/C2=O)Nc2ccc(N4CCN(CC5CN(c6cnc(C7CCC(=O)NC7=O)cc6C)C5)C[C@@H]4C)cc23)C1]

The titled compound was prepared in a manner similar to that in Example 33 with intermediate 17.

¹H NMR (500 MHz, DMSO) δ 12.39 (s, 1H), 10.74 (s, 1H), 8.64 (s, 1H), 7.88 (s, 1H), 7.62 (s, 1H), 7.45 (s, 1H), 7.34 (d, J=8.6 Hz, 1H), 7.00 (s, 1H), 6.94 (s, 1H), 6.86 (d, J=8.6 Hz, 1H), 4.98-4.15 (m, 4H), 4.13-4.06 (m, 2H), 3.90-3.85 (m, 1H), 3.84-3.79 (m, 1H), 3.70 (s, 3H), 3.64-3.58 (m, 2H), 3.24-3.15 (m, 2H), 3.04-2.87 (m, 5H), 2.64-2.52 (m, 10H), 2.48-2.43 (m, 2H), 2.31-2.25 (m, 1H), 2.20-2.12 (m, 4H), 2.11-2.03 (m, 1H), 0.95 (d, J=6.1 Hz, 3H). [M+H]⁺=798.7.

### Example 38: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)piperdine-1-yl)-3-fluoropyridin-2-yl)piperidine 6-dione

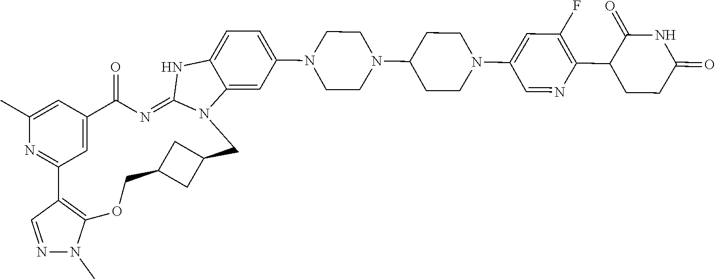 [SMILES: Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)c(F)c6)CC5)CC4)cc23)C1]

The titled compound was prepared in a manner similar to that in Example 6 with intermediate 9. The racemate was separated by Prep-Chiral-HPLC with following condition: (Column: CHIRALPAK IF, 2 cm×25 cm, 5 um; Flow rate: 20 mL/min;) to afford the title compound corresponded to peak A @254 nm (Ret. Time: 2.186 min). ¹H NMR (500 MHz, DMSO) δ 12.42 (s, 1H), 10.86 (s, 1H), 8.67 (s, 1H), 8.13 (s, 1H), 7.90 (s, 1H), 7.47 (s, 1H), 7.36 (d, J=8.5 Hz, 1H), 7.32-7.27 (m, 1H), 7.04 (s, 1H), 6.79 (d, J=8.6 Hz, 1H), 4.15-4.08 (m, 1H), 3.91-3.85 (m, 2H), 3.73-3.70 (m, 4H), 3.52 (s, 2H), 3.34-3.30 (m, 5H), 3.20-3.14 (m, 3H), 2.98-2.89 (m, 3H), 2.85-2.80 (m, 3H), 2.75-2.69 (m, 4H), 2.65 (s, 2H), 2.57 (s, 3H), 2.37 (s, 1H), 2.26-2.22 (m, 1H), 2.09-2.05 (m, 1H), 1.94-1.90 (s, 2H), 1.59-1.55 (m, 1H); [M+H]⁺=802.5.

### Example 40: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)piperidin-1-yl)-4-methylpyridin-2-yl)piperidine-2,6-dione

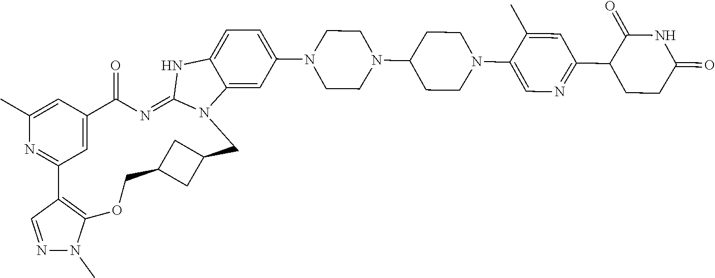 [SMILES: Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)CC4)cc23)C1]

The title compound was prepared in a procedure similar to that in Example 6 with intermediate 10. ¹H NMR (500 MHz, DMSO) δ 12.40 (s, 1H), 10.80 (s, 1H), 8.66 (s, 1H), 8.15 (s, 1H), 7.89 (s, 1H), 7.46 (s, 1), 7.35 (d, J=8.7 Hz, 1H), 7.14 (s, 1H), 7.02 (s, 1H), 6.90 (d, J=8.7 Hz, 1H), 4.81-4.01 (m, 4H), 3.89 (dd, J=8.7, 5.3 Hz, 1H), 3.72 (s, 4H), 3.20-3.15 (m, 6H), 2.92 (d, J=5.0 Hz, 2H), 2.76-2.66 (m, 6H), 2.64-2.52 (m, 7H), 2.40 (t, J=11.2 Hz, 1H), 2.31-2.07 (m, 6H), 1.92 (d, J=11.1 Hz, 2H), 1.67-1.56 (m, 2H). [M+H]⁺=798.6.

### Example 102: 3-(5-(4-(1-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperidin-4-yl)piperazin-1-yl)-4-methylpyridin-2-yl)piperidine-2,6-dione

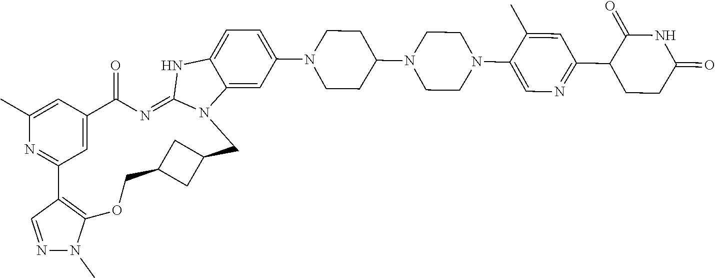 [SMILES: Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6C)CC5)CC4)cc23)C1]

The titled compound was prepared in a manner similar to that in Example 28.

¹H NMR (500 MHz, DMSO) δ 12.34 (s, 1H), 10.74 (s, 1H), 8.59 (s, 1H), 8.11 (s, 1H), 7.82 (s, 1H), 7.39 (s, 1H), 7.28 (d, J=8.7 Hz, 1H), 7.09 (s, 1H), 6.98 (s, 1H), 6.86 (d, J=8.8 Hz, 1H), 4.62-4.51 (m, 1H), 4.22-4.11 (m, 2H), 3.86-3.82 (m, 1H), 3.71 (s, 1H), 3.65 (s, 3H), 3.24-3.22 (m, 4H), 2.89-2.83 (m, 5H), 2.65 (t, J=11.4 Hz, 3H), 2.56 (d, J=11.2 Hz, 1H), 2.52-2.49 (m, 5H), 2.46-2.44 (m, 5H), 2.20 (s, 3H), 2.14-2.10 (m, 1H), 2.06-2.03 (m, 1H), 1.92-1.83 (m, 2H), 1.60-1.56 (m, 2H), 1.23-1.16 (m, 1H). [M+H]⁺=798.6.

### Example 70: (R)-3-(4-(4-(1-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperidin-4-yl)-3-oxopiperazin-1-yl)-2,6-difluorophenyl)piperidine-2,6-dione

### Step 1: tert-butyl 4-((2-(((benzyloxy)carbonyl)amino)ethyl)amino)piperidine-1-carboxylate

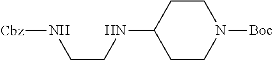 [SMILES: CC(C)(C)OC(=O)N1CCC(NCCNC(=O)OCc2ccccc2)CC1]

To a solution of tert-butyl 4-oxopiperidine-1-carboxylate (3.1 g, 15.0 mmol), benzyl (2-aminoethyl)carbamate (3.0 g, 15.0 mmol) and AcOH (1.5 g, 22.5 mmol) in DCE (100 mL) was added STAB (6.3 g, 30.0 mmol). Then the mixture was stirred at RT for 16 hours. The reaction was quenched with NaHCO₃/water (100 mL) and extracted with DCM (150 mL×2). The combined organic phase was washed with brine (100 mL), dried over Na₂SO₄, filtered and concentrated in vacuum. The residue was purified by silica gel column (DCM:CH₃OH=15:1), to afford the title product (5.5 g, 93.3%). [M+H]⁺=378.5.

### Step 2: tert-butyl 4-(N-(2-(((benzyloxy)carbonyl)amino)ethyl)-2-chloroacetamido)piperidine-1-carboxylate

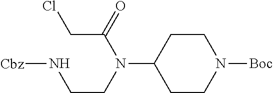 [SMILES: CC(C)(C)OC(=O)N1CCC(N(CCNC(=O)OCc2ccccc2)C(=O)CCl)CC1]

To a solution of tert-butyl 4-((2-(((benzyloxy)carbonyl)amino)ethyl)amino)piperidine-1-carboxylate (5.5 g, 14.5 mmol) and DIEA (5.6 g, 43.5 mmol) in DCM (150 mL) was added 2-chloroacetyl chloride (2.0 g, 17.4 mmol) cooled to 0° C. Then the mixture was stirred at RT for 3 hours. The reaction was quenched with water (100 mL) and extracted with DCM (150 mL×2). The combined organic phase was washed with brine (100 mL×1), dried over Na₂SO₄, filtered and concentrated in vacuum. The residue was purified by silica gel column (DCM:CH₃OH=20:1), to afford the title product (6.0 g, 90.9%). [M+H]⁺=454.5.

### Step 3: benzyl 4-(1-(tert-butoxycarbonyl)piperidin-4-yl)-3-oxopiperazine-1-carboxylate

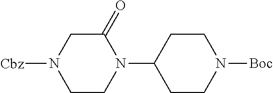 [SMILES: CC(C)(C)OC(=O)N1CCC(N2CCN(C(=O)OCc3ccccc3)CC2=O)CC1]

To a solution of tert-butyl 4-(N-(2-(((benzyloxy)carbonyl)amino)ethyl)-2-chloroacetamido)piperidine-1-carboxylate (6.0 g, 13.2 mmol) in DMF (80 mL) was added NaH (1.0 g, 26.4 mmol) at RT. Then the mixture was stirred at RT for 0.5 hour. The reaction was quenched with NH₄Cl/water (100 mL) and extracted with EA (150 mL×2). The combined organic phase was washed with brine (100 mL×2), dried over Na₂SO₄, filtered and concentrated in vacuum. The residue was purified by silica gel column (DCM:CH₃OH=20:1), to afford the title product (5.3 g, 96.3%). [M+H]⁺=418.5.

### Step 4: tert-butyl 4-(2-oxopiperazin-1-yl)piperidine-1-carboxylate

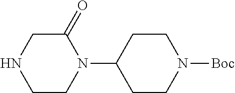 [SMILES: CC(C)(C)OC(=O)N1CCC(N2CCNCC2=O)CC1]

To the solution of benzyl 4-(1-(tert-butoxycarbonyl)piperidin-4-yl)-3-oxopiperazine-1-carboxylate (5.3 g, 12.7 mmol) in THF (100 mL) was added Pd/C (1.5 g, 10 wt. %, wet). The resulting mixture was stirred at RT for 3 hours under hydrogen atmosphere (balloon). The mixture was filtered by celite and washed with DCM. The filtrate was concentrated in vacuum to afford the desired product (3.4 g, 94.4%). [M+H]⁺=284.5.

### Step 5: tert-butyl 4-(4-(4-(2,6-bis(benzyloxy)pyridin-3-yl)-3,5-difluorophenyl)-2-oxopiperazin-1-yl)piperidine-1-carboxylate

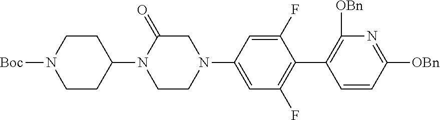 [SMILES: CC(C)(C)OC(=O)N1CCC(N2CCN(c3cc(F)c(-c4ccc(OCc5ccccc5)nc4OCc4ccccc4)c(F)c3)CC2=O)CC1]

To the solution of 2,6-bis(benzyloxy)-3-(4-bromo-2,6-difluorophenyl)pyridine (2.0 g, 4.1 mmol), tert-butyl 4-(2-oxopiperazin-1-yl)piperidine-1-carboxylate (1.8 g, 6.3 mmol) and Cs₂CO₃ (2.7 g, 8.2 mmol) in 100 mL 1,4-dioxane were added G3 RuPhos Pd (690 mg, 0.82 mmol) and RuPhos (383 mg, 0.82 mmol). The mixture was stirred at 100° C. for 16 hours. After LCMS showed the reaction was completed, the mixture was filtered through a celite pad and washed with DCM. The filtrate was concentrated under reduced pressure to give the crude residue, which was purified by silica column chromatography (PE:EA=50:1-1:1) to afford the product (2.6 g, 91.5%). [M+H]⁺=685.5.

### Step 6: tert-butyl 4-(4-(4-(2,6-dioxopiperidin-3-yl)-3,5-difluorophenyl)-2-oxopiperazin-1-yl)piperidine-1-carboxylate

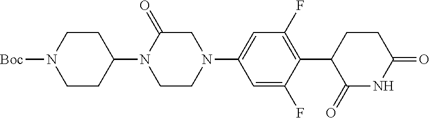 [SMILES: CC(C)(C)OC(=O)N1CCC(N2CCN(c3cc(F)c(C4CCC(=O)NC4=O)c(F)c3)CC2=O)CC1]

To the solution of tert-butyl 4-(4-(4-(2,6-bis(benzyloxy)pyridin-3-yl)-3,5-difluorophenyl)-2-oxopiperazin-1-yl)piperidine-1-carboxylate (1.1 g, 1.6 mmol) in 15 mL DMF and 15 mL i-PrOH, Pd/C (0.5 g, 10 wt. %, wet) was added. The mixture was stirred at 50° C. for 24 hours under hydrogen atmosphere (balloon). The mixture was cooled to room temperature and filtered by celite directly. The filtrate was concentrated in vacuum to afford the desired product (780 mg, 95.8%). [M+H]⁺=507.5

### Step 7: 3-(2,6-difluoro-4-(3-oxo-4-(piperidin-4-yl)piperazin-1-yl)phenyl)piperidine-2,6-dione

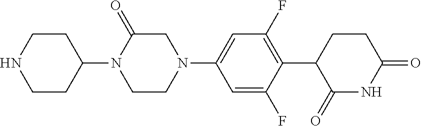 [SMILES: O=C1CCC(c2c(F)cc(N3CCN(C4CCNCC4)C(=O)C3)cc2F)C(=O)N1]

A solution of tert-butyl 4-(4-(4-(2,6-dioxopiperidin-3-yl)-3,5-difluorophenyl)-2-oxopiperazin-1-yl)piperidine-1-carboxylate (780 mg, 1.5 mmol) in HCl/1,4-dioxane (20 mL) was stirred at room temperature for 1 hour, the reaction mixture was concentrated under reduced pressure to afford the product (625 mg), which was used without further purification. [M+H]⁺=407.5 Step 8: (R)-3-(4-(4-(1-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperidin-4-yl)-3-oxopiperazin-1-yl)-2,6-difluorophenyl)piperidine-2,6-dione

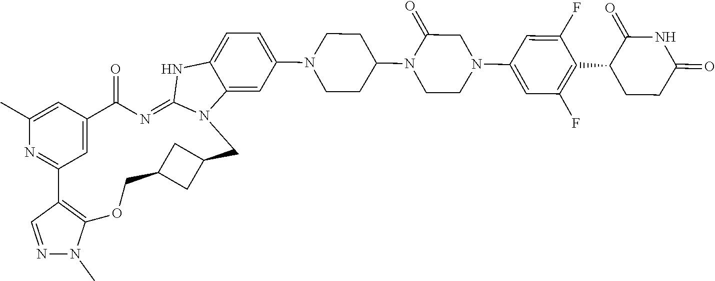 [SMILES: Cc1cc2cc(n1)-c1cnn(C)c1OC[C@H]1C[C@@H](CN3/C(=N/C2=O)Nc2ccc(N4CCC(N5CCN(c6cc(F)c([C@H]7CCC(=O)NC7=O)c(F)c6)CC5=O)CC4)cc23)C1]

To a stirred solution of intermediate 1 (50 mg, 0.1 mmol) in DMA (5 mL) were added 3-(2,6-difluoro-4-(3-oxo-4-(piperidin-4-yl)piperazin-1-yl)phenyl)piperidine-2,6-dione (80 mg, 0.2 mmol), t-BuONa (47 mg, 0.5 mmol), Ruphos (18 mg, 0.04 mmol) and Pd₂(dba)₃ (18 mg, 0.04 mmol) at room temperature under nitrogen atmosphere. The resulting mixture was stirred for 1 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with DCM/MeOH (15:1) to afford the crude product. Chiral separation by HPLC afforded the title compound (4.49 mg, 5.5%). HPLC conditions: Column: CHIRALPAK IA; Column Size: 2 cm×25 cm, 5 um; Mobile Phase:HEX:DCM (0.1% FA):MeOH=50:50; Flow Rate: 20 mL/min; Retention Time: 3.286 min.

¹H NMR (500 MHz, DMSO) δ 12.42 (s, 1H), 10.88 (s, 1H), 8.67 (s, 1H), 7.90 (s, 1H), 7.47 (s, 1H), 7.36 (d, J=8.9 Hz, 1H), 7.07 (s, 1H), 6.94 (d, J=8.1 Hz, 1H), 6.66 (s, 1H), 6.64 (s, 1H), 4.89-4.41 (m, 3H), 4.30-4.00 (m, 3H), 3.89 (s, 2H), 3.81 (d, J=11.1 Hz, 2H), 3.72 (s, 3H), 3.54-3.46 (m, 3H), 3.00-2.87 (m, 3H), 2.84-2.74 (m, 4H), 2.69-2.62 (m, 2H), 2.16-2.04 (m, 1H), 2.01-1.85 (m, 3H), 1.73-1.62 (m, 2H), 1.32-1.14 (m, 4H), 0.91-0.77 (m, 1H). [M+H]⁺=833.7.

### Example 41: 3-(5-(4-(3-(((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)(methyl)amino)azetidin-1-yl)piperidin-1-yl)-4-methylpyridin-2-yl)piperidine-2,6-dione

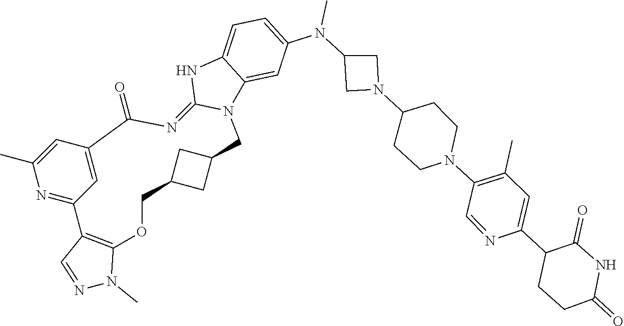 [SMILES: Cc1cc2cc(n1)-c1cnn(C)c1OC[C@H]1C[C@@H](CN3/C(=N/C2=O)Nc2ccc(N(C)C4CN(C5CCN(c6cnc(C7CCC(=O)NC7=O)cc6C)CC5)C4)cc23)C1]

The title compound was prepared in a manner similar to that in Example 6.

¹H NMR (500 MHz, DMSO) δ 12.28 (s, 1H), 10.73 (s, 1H), 8.58 (s, 1H), 8.06 (s, 1H), 7.82 (s, 1H), 7.39 (s, 1H), 7.28 (d, J=8.6 Hz, 1H), 7.06 (s, 1H), 6.76 (s, 1H), 6.66 (d, J=8.3 Hz, 1H), 4.95-3.87 (m, 6H), 3.81 (dd, J=8.8, 5.3 Hz, 1H), 3.65 (s, 3H), 3.60 (t, J=6.1 Hz, 2H), 3.07-2.99 (m, 2H), 2.88-2.82 (m, 4H), 2.76 (s, 3H), 2.66-2.60 (m, 2H), 2.53-2.46 (m, 5H), 2.21-2.08 (m, 6H), 2.06-2.00 (m, 1H), 1.81 (s, 1H), 1.75-1.68 (m, 3H), 1.35-1.25 (m, 2H). [M+H]⁺=798.30.

### Example 82: 3-(5-(2-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)ethyl)-6-methylpyridin-2-yl)piperidine-2,6-dione

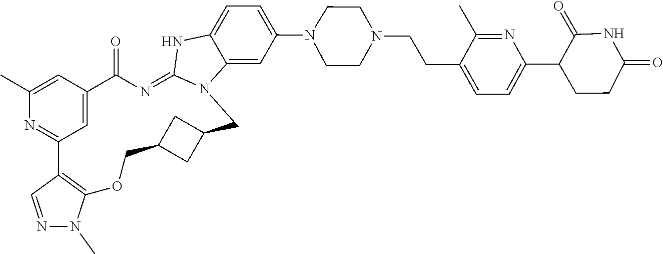 [SMILES: Cc1cc2cc(n1)-c1cnn(C)c1OC[C@H]1C[C@@H](CN3/C(=N/C2=O)Nc2ccc(N4CCN(CCc5ccc(C6CCC(=O)NC6=O)nc5C)CC4)cc23)C1]

To the solution of the product in example 6 step 2 (100 mg, 0.19 mmol), intermediate 27 (96 mg, 0.29 mmol) and KI (65 mg, 0.38 mmol) in MeCN (5 mL)/DMSO (2 mL) was added DIEA (75 mg, 0.57 mmol). The resulting mixture was heated at 80° C. overnight under N₂. The mixture was quenched with water and extracted with DCM (3×20 mL). The combined organic phase was washed with brine (1×15 mL), dried over Na₂SO₄, filtered and concentrated under reduced pressure to give the crude residue, which was purified by silica column chromatography (DCM:MeOH=100:1-10:1), followed by Prep-HPLC chromatography to afford the title product (31 mg, 21.4%).

¹H NMR (500 MHz, DMSO) δ 12.41 (s, 1H), 10.81 (s, 1H), 8.66 (s, 1H), 7.89 (s, 1H), 7.57 (d, J=7.8 Hz, 1H), 7.46 (s, 1H), 7.35 (d, J=8.7 Hz, 1H), 7.11 (d, J=7.8 Hz, 1H), 7.03 (s, 1H), 6.91 (d, J=8.8 Hz, 1H), 4.88-3.99 (m, 4H), 3.92 (dd, J=9.5, 5.3 Hz, 1H), 3.72 (s, 3H), 3.22-3.14 (m, 4H), 2.92 (s, 2H), 2.79 (t, J=7.5 Hz, 2H), 2.68-2.51 (m, 14H), 2.46 (s, 3H), 2.23 (dd, J=9.0, 4.6 Hz, 2H), 2.13-2.06 (m, 1H). [M+H]⁺=743.4.

### Example 62: 3-(5-((3S,4R)-4-(4-((7¹s,7³S,E)-1¹,2⁶-dimethyl-3-oxo-5²,5³-dihydro-1¹H,5¹H-9-oxa-4- aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-6-methylpyridin-2-yl)piperidine-2,6-dione

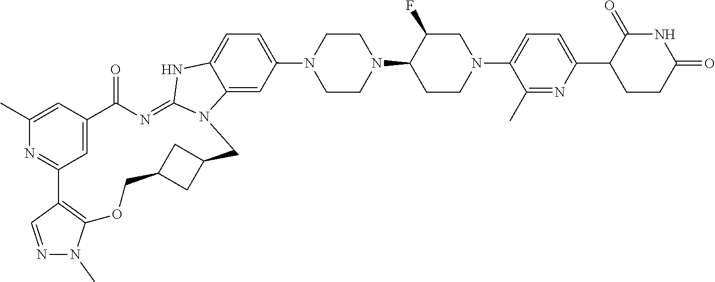 [SMILES: Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)C[C@@H]5F)CC4)cc23)C1]

The title compound was prepared in a manner similar to that in Example 26 step 4 with intermediates 1 and 28. ¹H NMR (500 MHz, DMSO) δ 12.34 (s, 1H), 10.72 (s, 1H), 8.59 (s, 1H), 7.85-7.79 (m, 1H), 7.39 (s, 1H), 7.30 (dd, J=16.3, 8.2 Hz, 2H), 7.05 (d, J=7.7 Hz, 1H), 6.97 (s, 1H), 6.85 (d, J=8.5 Hz, 1H), 5.04-4.99 (m, 1H), 4.63-4.58 (m, 2H), 4.24-3.93 (m, 2H), 3.83-3.78 (m, 1H), 3.65-3.59 (m, 4H), 3.10-3.01 (m, 5H), 2.98-2.67 (m, 11H), 2.57-2.48 (m, 6H), 2.32-2.28 (m, 3H), 2.24-2.08 (m, 2H), 2.06-1.90 (m, 2H), 1.82-1.72 (m, 1H); [M+H]⁺=816.5.

### Example 43: 3-(5-(4-((1R,4R)-5-((7¹s,7³S,E)-1¹,2⁶-dimethyl-3-oxo-5²,5³-dihydro-1¹H,5¹H-9-oxa-4- aza-5(2,1)-benzo[d]imidazola-2(2,4)-pyridina-1(4,5)-pyrazola-7(1,3)-cyclobutanacyclononaphane-5⁶-yl)-2,5-diazabicyclo[2.2.1]heptan-2-yl)piperidin-1-yl)-6-methylpyridin-2-yl)piperidine-2,6-dione

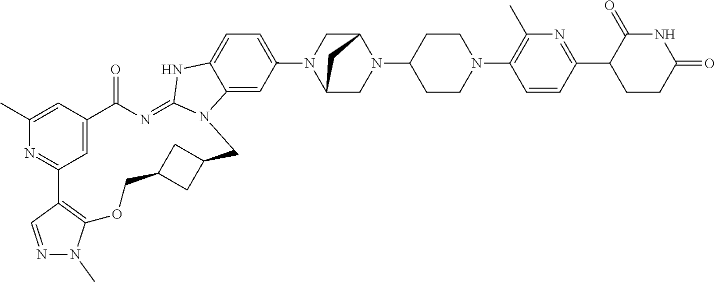 [SMILES: Cc1cc2cc(n1)-c1cnn(C)c1OC[C@H]1C[C@@H](CN3/C(=N/C2=O)Nc2ccc(N4C[C@H]5C[C@@H]4CN5C4CCN(c5ccc(C6CCC(=O)NC6=O)nc5C)CC4)cc23)C1]

The title compound was prepared in a manner similar to that in Example 6 with intermediate 12. ¹H NMR (500 MHz, DMSO) δ 12.33 (s, 1H), 10.77 (s, 1H), 8.66 (s, 1H), 7.89 (s, 1H), 7.46 (s, 1H), 7.32 (d, J=8.5 Hz, 2H), 7.08 (d, J=8.2 Hz, 1H), 6.65 (s, 1H), 6.55 (d, J=8.7 Hz, 1H), 4.49-4.39 (m, 1H), 3.97-3.84 (m, 2H), 3.72 (s, 3H), 3.50-3.40 (m, 2H), 3.25-2.88 (m, 8H), 2.61-2.50 (m, 11H), 2.45-2.32 (m, 5H), 2.26-2.14 (m, 2H), 2.10-2.01 (m, 1H), 1.96-1.85 (i, 4H), 1.62-1.42 (m, 2H); [M+H]⁺=810.5.

### Example 109: 3-(5-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl-2-(methoxymethyl)piperazin-1-yl)piperidin-1-yl)-6-methylpyridin-2-yl)piperidine-2,6-dione

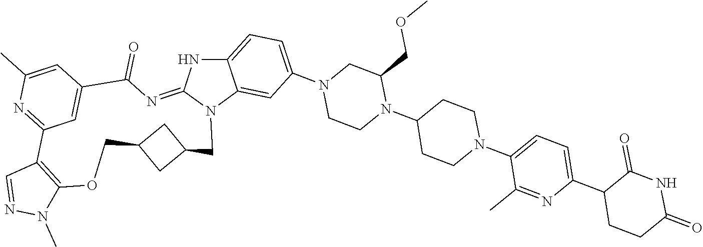 [SMILES: COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2ccc(C3CCC(=O)NC3=O)nc2C)CC1]

The title compound was prepared in a manner similar to that in Example 30 with intermediate 12. ¹H NMR (500 MHz, DMSO) δ 12.32 (s, 1H), 10.80 (s, 1H), 8.66 (s, 1H), 8.11 (s, 1H), 7.89 (s, 1H), 7.46 (s, 1H), 7.30 (d, J=8.7 Hz, 1H), 7.12 (s, 1H), 6.92 (s, 1H), 6.79 (d, J=8.8 Hz, 1H), 3.92-3.77 (i, 2H), 3.72 (s, 3H), 3.57 (s, 1H), 3.51 (s, 1H), 3.44-3.40 (m, 1H), 3.18 (s, 2H), 3.04 (s, 2H), 2.94-2.88 (m, 6H), 2.76-2.73 (m, 3H), 2.63-2.60 (m, 2H), 2.56 (s, 3H), 2.55-2.51 (m, 6H), 2.36 (s, 1H), 2.28 (s, 1H), 2.26 (s, 3H), 2.24-2.16 (m, 2H), 2.10 (d, J=12.6 Hz, 2H), 1.92 (s, 1H), 1.77 (s, 2H), 1.57 (d, J=11.5 Hz, 1H). [M+H]⁺=842.6.

### Example 110: 3-(5-((S)-4-(1-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperidin-4-yl)-3-methylpiperazin-1-yl)-4-methylpyridin-2-yl)piperidine-2,6-dione

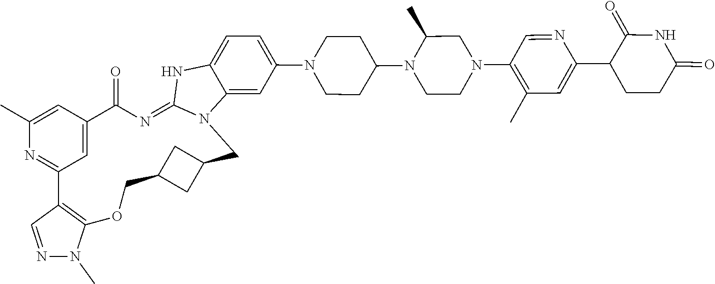 [SMILES: Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6C)C[C@@H]5C)CC4)cc23)C1]

The title compound was prepared in a manner similar to that in Example 17 with intermediate 22. ¹H NMR (500 MHz, DMSO) δ 12.40 (s, 1H), 10.80 (s, 1H), 8.66 (s, 1H), 8.13 (s, 1H), 7.90 (s, 1H), 7.46 (s, 1H), 7.34 (d, J=8.7 Hz, 1H), 7.14 (s, 1H), 7.03 (s, 1H), 6.95-6.88 (m, 1H), 4.82-4.02 (m, 4H), 3.89 (dd, J=8.8, 5.4 Hz, 1H), 3.78-3.70 (m, 5H), 3.02-2.82 (m, 9H), 2.78-2.53 (m, 11H), 2.27 (s, 3H), 2.19 (dd, J=8.5, 4.7 Hz, 1H), 2.14-2.07 (m, 1H), 1.88-1.70 (m, 4H), 1.60-1.51 (m, 1H), 1.11 (d, J=6.0 Hz, 3H). [M+H]⁺=812.7.

Example 57: 3-(5-((3R,4R)-4-(4-((7¹s,7³S,E)-1¹,2⁶-dimethyl-3-oxo-5²,5³-dihydro-1¹H,5¹H-9-oxa-4- aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-6-methylpyridin-2-yl)piperidine-2,6-dione

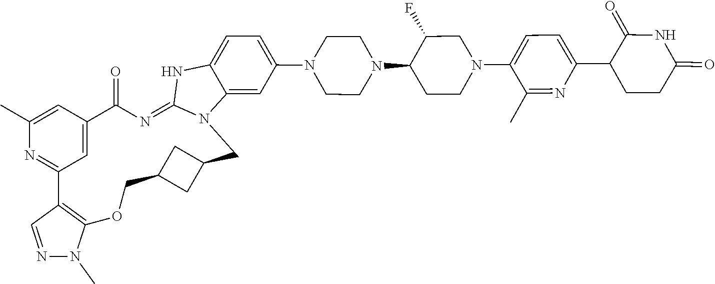 [SMILES: Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)C[C@H]5F)CC4)cc23)C1]

The title compound was prepared in a manner similar to that in Example 26 step 4 with intermediates 1 and 29. ¹H NMR (500 MHz, DMSO) δ 12.41 (s, 1H), 10.79 (s, 1H), 8.66 (s, 1H), 7.89 (s, 1H), 7.48-7.40 (m, 2H), 7.35 (d, J=8.6 Hz, 1H), 7.13 (d, J=8.1 Hz, 1H), 7.03 (s, 1H), 6.92 (d, J=9.1 Hz, 1H), 4.94-4.04 (m, 5H), 3.91 (dd, J=9.4, 5.2 Hz, 1H), 3.72 (s, 3H), 3.44-3.36 (m, 2H), 3.17 (s, 4H), 3.09-3.05 (m, 1H), 2.95-2.83 (m, 5H), 2.79-2.72 (m, 2H), 2.68-2.53 (m, 8H), 2.43-2.36 (m, 4H), 2.25-2.17 (m, 1H), 2.12-2.05 (m, 1H), 1.94-1.88 (m, 1H), 1.76-1.70 (m, 1H). [M+H]⁺=816.60.

### Example 112: 3-(5-(4-(6-(((7¹s,7³s,E)-1¹,2⁶-dimethyl-3-oxo-5²,5³-dihydro-1¹′H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)(methyl)amino)-2-azaspiro[3.3]heptan-2-yl)piperidin-1-yl)-6-methylpyridin-2-yl)piperidine-2,6-dione

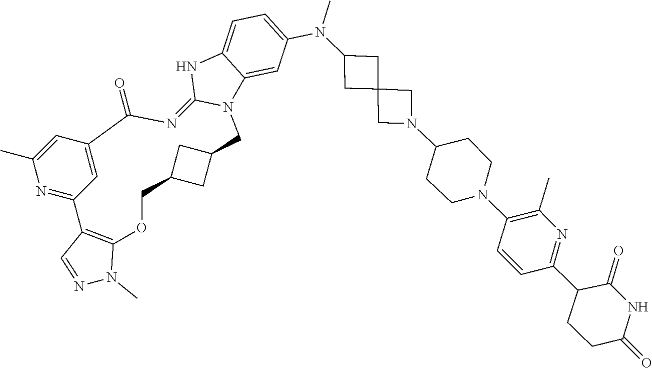 [SMILES: Cc1cc2cc(n1)-c1cnn(C)c1OC[C@H]1C[C@@H](CN3/C(=N/C2=O)Nc2ccc(N(C)C4CC5(C4)CN(C4CCN(c6ccc(C7CCC(=O)NC7=O)nc6C)CC4)C5)cc23)C1]

The title compound was prepared in a manner similar to that in Example 6 with intermediate 12. ¹H NMR (500 MHz, DMSO) δ 12.38 (s, 1H), 10.78 (s, 1H), 8.65 (s, 1H), 7.90 (s, 1H), 7.46 (s, 1H), 7.33 (d, J=8.5 Hz, 2H), 7.08 (d, J=8.0 Hz, 1H), 6.81-6.70 (m, 2H), 4.90-3.80 (m, 8H), 3.72 (s, 3H), 3.27 (s, 2H), 3.12-2.86 (m, 8H), 2.76 (s, 3H), 2.59-2.54 (m, 6H), 2.45-2.39 (m, 2H), 2.36 (s, 3H), 2.25-2.15 (m, 2H), 2.13-2.04 (m, 4H), 1.75-1.68 (m, 2H), 1.38-1.27 (m, 2H). [M+H]⁺=838.70.

### Example 113: 3-(5-(4-(6-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2,6-diazaspiro[3.3]heptan-2-yl)piperidin-1-yl)-6-methylpyridin-2-yl)piperidine-2,6-dione

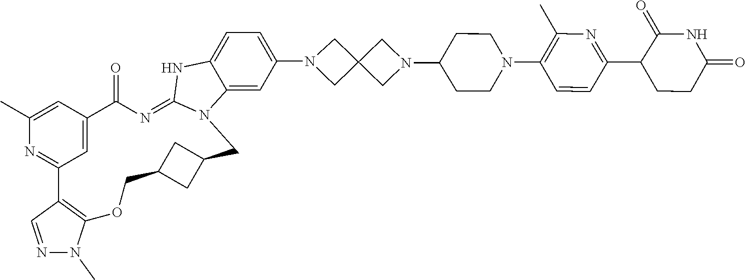 [SMILES: Cc1cc2cc(n1)-c1cnn(C)c1OC[C@H]1C[C@@H](CN3/C(=N/C2=O)Nc2ccc(N4CC5(C4)CN(C4CCN(c6ccc(C7CCC(=O)NC7=O)nc6C)CC4)C5)cc23)C1]

The title compound was prepared in a manner similar to that in Example 6 with intermediate 12. ¹H NMR (500 MHz, DMSO) δ 12.31 (s, 1H), 10.71 (s, 1H), 8.58 (s, 1H), 7.82 (s, 1H), 7.39 (s, 1H), 7.33-7.26 (m, 2H), 7.03 (d, J=8.0 Hz, 1H), 6.43 (s, 1H), 6.29 (d, J=8.5 Hz, 1H), 4.82-3.79 (m, 11H), 3.65 (s, 3H), 3.06-2.79 (m, 8H), 2.56-2.47 (m, 10H), 2.31 (s, 3H), 2.19-2.09 (m, 2H), 2.05-1.98 (m, 1H), 1.84 (s, 1H), 1.78-1.68 (m, 1H), 1.36-1.26 (m, 1H). [M+H]⁺=810.7.

### Example 114: 3-(5-(4-(3-((((7¹s,7³s,E)-1¹,2⁶-dimethyl-3-oxo-5²,5³-dihydro-1′H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)(methyl)amino)methyl)azetidin-1-yl)piperidin-1-yl)-6-methylpyridin-2-yl)piperidine-2,6-dione

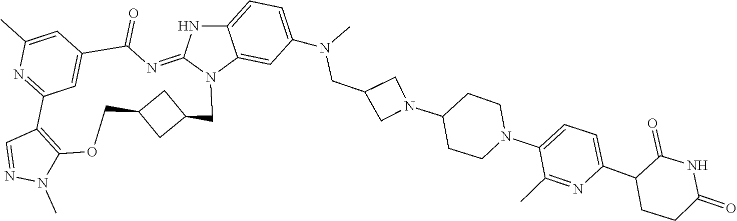 [SMILES: Cc1cc2cc(n1)-c1cnn(C)c1OC[C@H]1C[C@@H](CN3/C(=N/C2=O)Nc2ccc(N(C)CC4CN(C5CCN(c6ccc(C7CCC(=O)NC7=O)nc6C)CC5)C4)cc23)C1]

The titled compound was prepared in a manner similar to that in Example 6 with intermediate 12. ¹H NMR (500 MHz, DMSO) δ 12.33 (s, 1H), 10.77 (s, 1H), 8.65 (s, 1H), 7.89 (s, 1H), 7.44 (d, J=12.1 Hz, 1H), 7.36-7.31 (m, 2H), 7.09 (d, J=8.2 Hz, 1H), 6.86-6.59 (m, 2H), 3.89-3.85 (m, 1H), 3.71 (s, 3H), 3.53 (s, 3H), 3.02 (s, 3H), 2.91 (s, 8H), 2.73-2.58 (m, 5H), 2.55 (s, 7H), 2.37 (s, 4H), 2.18 (s, 3H), 2.10-2.00 (m, 1H), 1.73 (s, 3H), 1.33 (d, J=9.2 Hz, 2H); [M+H]⁺=812.4.

### Example 47: 3-(5-((1S,4S)-5-(1-((7¹s,7³R,E)-1¹,2⁶-dimethyl-3-oxo-5²,5³-dihydro-1′H,5¹H-9-oxa-4- aza-5(2,1)-benzo[d]imidazola-2(2,4)-pyridina-1(4,5)-pyrazola-7(1,3)-cyclobutanacyclononaphane-5⁶-yl)piperidin-4-yl)-2,5-diazabicyclo[2.2.1]heptan-2-yl)-6-methylpyridin-2-yl)piperidine-2,6-dione

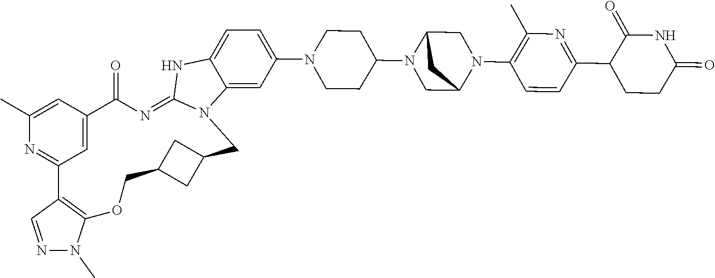 [SMILES: Cc1cc2cc(n1)-c1cnn(C)c1OC[C@H]1C[C@@H](CN3/C(=N/C2=O)Nc2ccc(N4CCC(N5C[C@@H]6C[C@H]5CN6c5ccc(C6CCC(=O)NC6=O)nc5C)CC4)cc23)C1]

The title compound was prepared in a manner similar to that in Example 28 with intermediate 23. ¹H NMR (500 MHz, DMSO) δ 12.39 (s, 1H), 10.76 (s, 1H), 8.65 (s, 1H), 7.89 (s, 1H), 7.46 (s, 1H), 7.32 (d, J=8.6 Hz, 1H), 7.11-7.04 (m, 1H), 6.99 (d, J=7.6 Hz, 2H), 6.88 (d, J=9.0 Hz, 1H), 4.82-4.47 (m, 2H), 4.25-3.97 (m, 3H), 3.83-3.79 (m, 1H), 3.76 (s, 1H), 3.61-3.53 (m, 2H), 2.98-2.83 (m, 3H), 2.81-2.71 (m, 2H), 2.64-2.54 (m, 7H), 2.41-2.24 (m, 9H), 2.24-2.03 (m, 4H), 1.92-1.66 (m, 5H), 1.52-1.38 (m, 2H); [M+H]⁺=810.5.

### Example 117: 3-(5-(4-((1S,4S)-5-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2,5-diazabicyclo[2,2,1]heptan-2-yl)piperidin-1-yl)-6-methylpyridin-2-yl)piperidine-2,6-dione

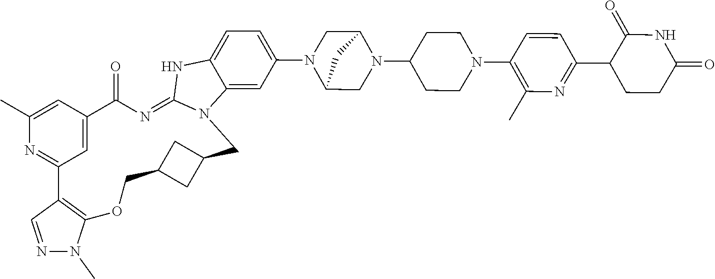 [SMILES: Cc1cc2cc(n1)-c1cnn(C)c1OC[C@H]1C[C@@H](CN3/C(=N/C2=O)Nc2ccc(N4C[C@@H]5C[C@H]4CN5C4CCN(c5ccc(C6CCC(=O)NC6=O)nc5C)CC4)cc23)C1]

The titled compound was prepared in a manner similar to that in Example 6 with intermediate 12. ¹H NMR (500 MHz, DMSO) δ 12.32 (s, 1H), 10.77 (s, 1H), 8.66 (s, 1H), 7.89 (s, 1H), 7.46 (s, 1H), 7.32 (d, J=8.5 Hz, 2H), 7.08 (d, J=7.9 Hz, 1H), 6.65 (s, 1H), 6.55 (d, J=8.3 Hz, 1H), 4.46-4.39 (m, 2H), 3.87 (dd, J=9.3, 5.3 Hz, 1H), 3.72 (s, 3H), 3.39-3.32 (m, 2H), 3.31-3.24 (m, 4H), 3.13-2.87 (m, 6H), 2.65-2.58 (m, 3H), 2.56-2.47 (m, 6H), 2.37 (s, 4H), 2.19 (d, J=9.7 Hz, 2H), 2.06 (dd, J=13.2, 5.7 Hz, 2H), 1.93-1.87 (m, 4H), 1.57-1.52 (m, 2H); [M+H]⁺=810.4

### Example 44: 3-(5-((S)-4-(1-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperidin-4-yl)-3-methylpiperazin-1-yl)-6-methylpyridin-2-yl)piperidine-2,6-dione

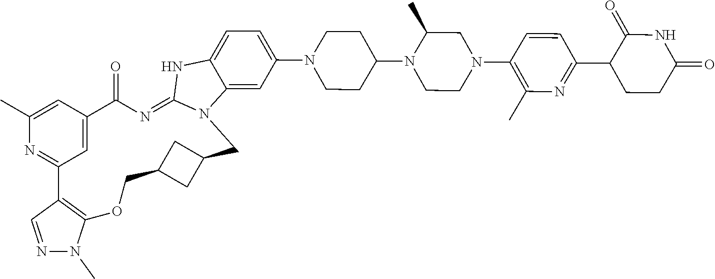 [SMILES: Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)C[C@@H]5C)CC4)cc23)C1]

The titled compound was prepared in a manner similar to that in Example 17 with intermediates 25 and 30. ¹H NMR (500 MHz, DMSO) δ 12.41 (s, 1H), 10.79 (s, 1H), 8.66 (s, 1H), 7.89 (s, 1H), 7.46 (s, 1H), 7.38 (d, J=7.8 Hz, 1H), 7.35 (d, J=8.6 Hz, 1H), 7.13 (d, J=8.1 Hz, 1H), 7.04 (s, 1H), 6.92 (d, J=9.1 Hz, 1H), 4.24-4.02 (m, 2H), 3.92-3.88 (m, 1H), 3.72 (s, 3H), 3.27-3.18 (m, 4H), 2.94-2.90 (m, 4H), 2.77 (m, 2H), 2.57-2.55 (m, 5H), 2.44-2.41 (m, 5H), 2.23-2.16 (m, 2H), 2.12-2.06 (m, 1H), 1.81-1.75 (m, 2H), 1.51-1.45 (m, 3H), 1.27-1.21 (m, 6H), 0.89-0.81 (m, 3H). [M+H]⁺=812.7.

### Example 120: 3-(5-(4-((S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-(methoxymethyl)piperazin-1-yl)piperidin-1-yl)-4-methylpyridin-2-yl)piperidine-2,6-dione

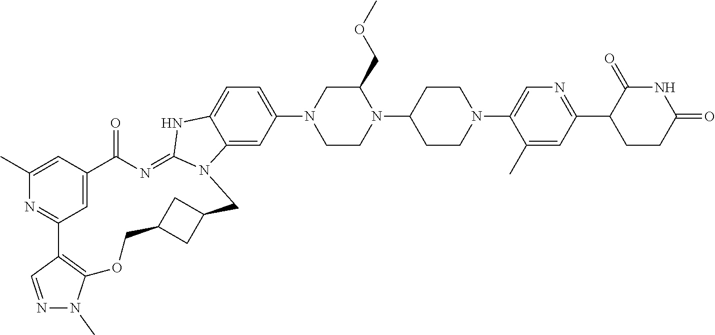 [SMILES: COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2cnc(C3CCC(=O)NC3=O)cc2C)CC1]

The titled compound was prepared in a manner similar to that in Example 6 with intermediates 1 and 10. ¹H NMR (500 MHz, DMSO) δ 12.41 (s, 1H), 10.79 (s, 1H), 8.66 (s, 1H), 8.15 (s, 1H), 7.89 (s, 1H), 7.46 (s, 1H), 7.36 (d, J=8.8 Hz, 1H), 7.14 (s, 1H), 6.98 (s, 1H), 6.88 (d, J=8.7 Hz, 1H), 3.92-3.77 (m, 2H), 3.72 (s, 3H), 3.57 (s, 1H), 3.51 (s, 1H), 3.44-3.40 (m, 1H), 3.18 (s, 2H), 3.04 (s, 2H), 2.94-2.88 (m, 6H), 2.76-2.73 (m, 3H), 2.63-2.60 (m, 2H), 2.56 (s, 3H), 2.55-2.51 (m, 6H), 2.36 (s, 1H), 2.28 (s, 1H), 2.26 (s, 3H), 2.24-2.16 (m, 2H), 2.10 (d, J=12.6 Hz, 2H), 1.92 (s, 1H), 1.77 (s, 2H), 1.57 (d, J=11.5 Hz, 1H). [M+H]⁺=842.0.

### Example 121: 3-(5-(4-(3-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)azetidin-1-yl)piperidin-1-yl)-6-methylpyridin-2-yl)piperidine-2,6-dione

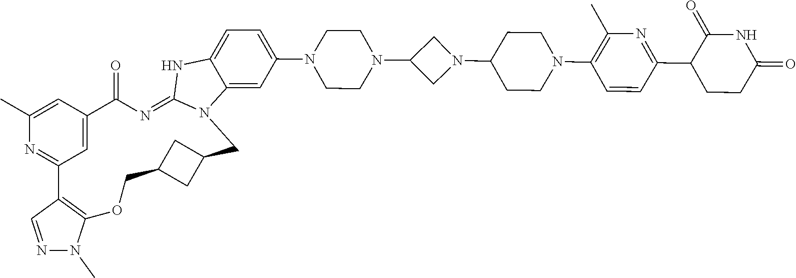 [SMILES: Cc1cc2cc(n1)-c1cnn(C)c1OC[C@H]1C[C@@H](CN3/C(=N/C2=O)Nc2ccc(N4CCN(C5CN(C6CCN(c7ccc(C8CCC(=O)NC8=O)nc7C)CC6)C5)CC4)cc23)C1]

The titled compound was prepared in a manner similar to that in Example 6 with intermediates 39 and 12. ¹H NMR (500 MHz, DMSO) δ 12.36 (s, 1H), 10.78 (s, 1H), 8.65 (s, 1H), 7.89 (s, 1H), 7.46 (s, 1H), 7.35 (dd, J=8.3, 3.5 Hz, 2H), 7.09 (d, J=8.1 Hz, 1H), 7.02 (s, 1H), 6.90 (d, J=8.5 Hz, 1H), 4.62-4.51 (m, 2H), 4.29-4.18 (m, 2H), 3.88 (dd, J=9.3, 5.3 Hz, 1H), 3.72 (s, 3H), 3.43 (s, 2H), 3.16 (s, 5H), 3.04-3.01 (m, 2H), 2.93-2.87 (m, 6H), 2.63-2.58 (m, 3H), 2.56 (s, 4H), 2.43 (s, 5H), 2.37 (s, 3H), 2.26-2.14 (m, 3H), 2.11-2.03 (m, 1H), 1.81-1.73 (m, 2H), 1.38-1.35 (m, 2H). [M+H]⁺=853.8.

### Example 122: 3-(5-(4-((S)-1-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3,3-difluoropiperidin-4-yl)piperazin-1-yl)-6-methylpyridin-2-yl)piperidine-2,6-dione

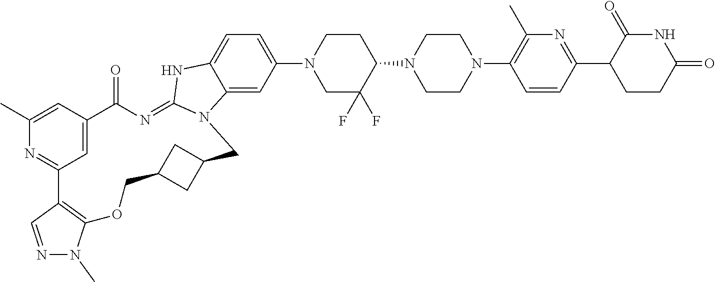 [SMILES: Cc1cc2cc(n1)-c1cnn(C)c1OC[C@H]1C[C@@H](CN3/C(=N/C2=O)Nc2ccc(N4CC[C@H](N5CCN(c6ccc(C7CCC(=O)NC7=O)nc6C)CC5)C(F)(F)C4)cc23)C1]

The titled compound was prepared in a manner similar to that in Example 26 step 4 with intermediates 1 and 40. ¹H NMR (500 MHz, DMSO) δ 12.51 (s, 1H), 10.90 (s, 1H), 8.79 (s, 1H), 8.03 (s, 1H), 7.63 (s, 2H), 7.36 (d, J=8.8 Hz, 1H), 7.33 (s, 1H), 7.15 (s, 1H), 6.98 (d, J=8.6 Hz, 1H), 4.59-4.57 (m, 2H), 4.01-3.99 (m, 2H), 3.75 (s, 3H), 3.30-3.26 (m, 9H), 3.15-3.12 (m, 6H), 2.91-2.88 (m, 5H), 2.65 (s, 3H), 2.48 (s, 3H), 2.26 (d, J=8.4 Hz, 2H), 2.15-1.91 (m, 4H). [M+H]⁺=834.0.

### Example 123: 3-(5-((R)-4-(4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)-3,3-difluoropiperidin-1-yl)-6-methylpyridin-2-yl)piperidine-2,6-dione

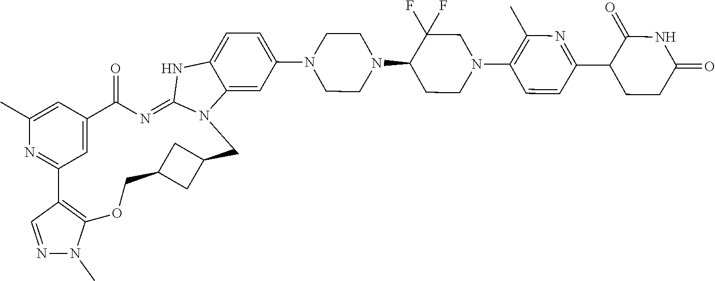 [SMILES: Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)CC5(F)F)CC4)cc23)C1]

The titled compound was prepared in a manner similar to that in Example 26 step 4 with intermediates 1 and 24. ¹H NMR (500 MHz, DMSO) δ 12.54 (s, 1H), 10.87 (s, 1H), 8.81 (s, 1H), 8.05 (s, 1H), 7.66 (s, 1H), 7.57 (s, 1H), 7.40 (d, J=8.7 Hz, 1H), 7.28 (s, 1H), 7.12 (s, 1H), 6.97 (d, J=8.9 Hz, 1H), 4.59-4.57 (m, 2H), 4.01-3.99 (m, 2H), 3.75 (s, 3H), 3.30-3.26 (m, 9H), 3.15-3.12 (m, 6H), 2.91-2.88 (m, 5H), 2.65 (s, 3H), 2.48 (s, 3H), 2.26 (d, J=8.4 Hz, 2H), 2.15-1.91 (m, 4H). [M+H]⁺=834.0.

### Example 124: 3-(5-(4-(5-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2-oxa-5,8-diazaspiro[3.5]nonan-8-yl)piperidin-1-yl)-6-methylpyridin-2-yl)piperidine-2,6-dione

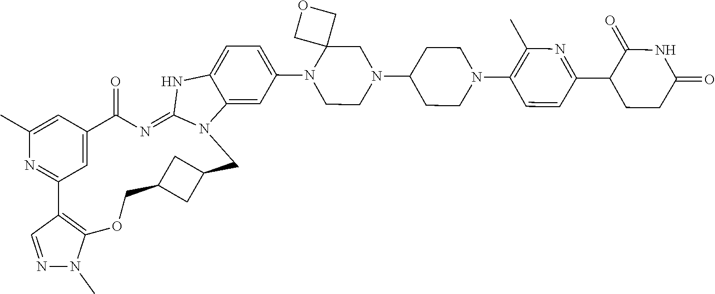 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC45COC5)cc23)C1]

The title compound was prepared in a manner similar to that in Example 6 with intermediate 12. ¹H NMR (500 MHz, DMSO) δ 12.50 (s, 1H), 10.78 (s, 1H), 8.66 (s, 1H), 7.89 (s, 1H), 7.47 (s, 1H), 7.42-7.33 (m, 2H), 7.10 (d, J=8.0 Hz, 1H), 7.03 (s, 1H), 6.76 (d, J=8.0 Hz, 1H), 4.83-4.47 (m, 4H), 4.44-4.03 (m, 5H), 3.92-3.85 (m, 1H), 3.72 (s, 3H), 3.24-3.19 (m, 3H), 3.13-3.09 (m, 3H), 3.06-2.99 (m, 3H), 2.97-2.84 (m, 4H), 2.64-2.59 (m, 6H), 2.20-2.16 (m, 3H), 2.07-2.01 (m, 2H), 1.88-1.81 (m, 3H), 1.72-1.59 (m, 3H). [M+H]⁺=840.5.

### Example 125: 3-(5-((S)-4-(4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)-3,3-difluoropiperidin-1-yl)-6-methylpyridin-2-yl)piperidine-2,6-dione

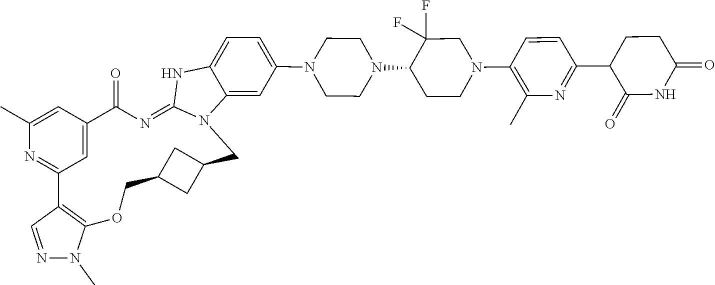 [SMILES: Cc1cc2cc(n1)-c1cnn(C)c1OC[C@H]1C[C@@H](CN3/C(=N/C2=O)Nc2ccc(N4CCN([C@H]5CCN(c6ccc(C7CCC(=O)NC7=O)nc6C)CC5(F)F)CC4)cc23)C1]

The titled compound was prepared in a manner similar to that in Example 26 step 4 with intermediates 1 and 31. ¹H NMR (500 MHz, DMSO) δ 12.54 (s, 1H), 10.87 (s, 1H), 8.81 (s, 1H), 8.05 (s, 1H), 7.66 (s, 1H), 7.57 (s, 1H), 7.40 (d, J=8.7 Hz, 1H), 7.28 (s, 1H), 7.12 (s, 1H), 6.97 (d, J=8.9 Hz, 1H), 4.59-4.57 (m, 2H), 4.01-3.99 (m, 2H), 3.75 (s, 3H), 3.30-3.26 (m, 9H), 3.15-3.12 (m, 6H), 2.91-2.88 (m, 5H), 2.65 (s, 3H), 2.48 (s, 3H), 2.26 (d, J=8.4 Hz, 2H), 2.15-1.91 (m, 4H). [M+H]⁺=834.0.

### Example 126: 3-(5-(4-(5-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2,5-diazabicyclo[2.2.2]octan-2-yl)piperidin-1-yl)-6-methylpyridin-2-yl)piperidine-2,6-dione

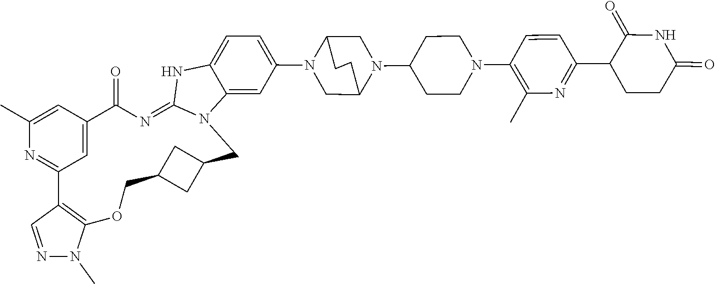 [SMILES: Cc1cc2cc(n1)-c1cnn(C)c1OC[C@H]1C[C@@H](CN3/C(=N/C2=O)Nc2ccc(N4CC5CCC4CN5C4CCN(c5ccc(C6CCC(=O)NC6=O)nc5C)CC4)cc23)C1]

The title compound was prepared in a manner similar to that in Example 6 with intermediate 12. ¹H NMR (500 MHz, DMSO) δ 12.29 (s, 1H), 10.78 (s, 1H), 8.65 (s, 1H), 7.89 (s, 1H), 7.44 (d, J=11.9 Hz, 1H), 7.35-7.31 (m, 2H), 7.09 (d, J=8.2 Hz, 1H), 6.68 (s, 1H), 6.60-6.58 (m, 1H), 4.13-4.08 (m, 3H), 3.91-3.86 (m, 1H), 3.71 (s, 3H), 3.63-3.59 (m, 1H), 3.26-3.17 (m, 4H), 3.10-3.02 (m, 2H), 3.00-2.86 (m, 5H), 2.66-2.62 (m, 2H), 2.60-2.53 (m, 6H), 2.37-2.32 (m, 4H), 2.25-2.19 (m, 2H), 2.13-1.90 (m, 5H), 1.88-1.83 (m, 1H), 1.81-1.74 (m, 1H), 1.64-1.55 (m, 1H), 1.52-1.47 (m, 2H). [M+H]⁺=824.5.

### Example 127: 3-(5-(4-(8-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3,8-diazabicyclo[3.2.1]octan-3-yl)piperidin-1-yl)-6-methylpyridin-2-yl)piperidine-2,6-dione

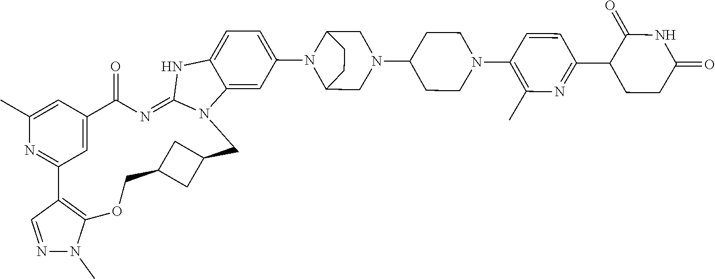 [SMILES: Cc1cc2cc(n1)-c1cnn(C)c1OC[C@H]1C[C@@H](CN3/C(=N/C2=O)Nc2ccc(N4C5CCC4CN(C4CCN(c6ccc(C7CCC(=O)NC7=O)nc6C)CC4)C5)cc23)C1]

The title compound was prepared in a manner similar to that in Example 6 with intermediate 12. ¹H NMR (500 MHz, DMSO) δ 12.33 (s, 1H), 10.77 (s, 1H), 8.66 (s, 1H), 7.89 (s, 1H), 7.46 (s, 1H), 7.32 (dd, J=12.2, 8.5 Hz, 2H), 7.08 (d, J=8.2 Hz, 1H), 6.92 (s, 1H), 6.79 (d, J=8.4 Hz, 1H), 4.87-4.44 (m, 2H), 4.32 (s, 2H), 4.26-3.95 (m, 2H), 3.87 (dd, J=9.4, 5.2 Hz, 1H), 3.72 (s, 3H), 3.06 (d, J=10.8 Hz, 2H), 2.91 (s, 3H), 2.67-2.53 (m, 13H), 2.37 (s, 3H), 2.26-2.13 (m, 3H), 2.10-2.01 (m, 1H), 1.93-1.78 (m, 6H), 1.60-1.48 (m, 2H). [M+H]⁺=824.5.

### Example 128: (3R)-3-(4-(4-(8-((7¹s,7³s,E)-1¹,2⁶-dimethyl-3-oxo-5²,5³-dihydro-1¹H,5¹H-9-oxa-4-aza- 5(2,1)-benzo[d]imidazola-2(2,4)-pyridina-1(4,5)-pyrazola-7(1,3)-cyclobutanacyclononaphane-5⁶-yl)-3,8-diazabicyclo[3.2.1]octan-3-yl)piperidin-1-yl)-2,6-difluorophenyl)piperidine-2,6-dione

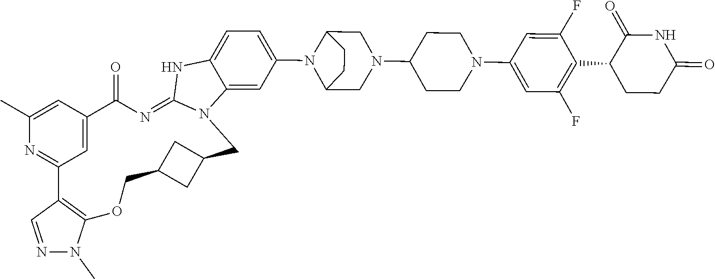 [SMILES: Cc1cc2cc(n1)-c1cnn(C)c1OC[C@H]1C[C@@H](CN3/C(=N/C2=O)Nc2ccc(N4C5CCC4CN(C4CCN(c6cc(F)c([C@H]7CCC(=O)NC7=O)c(F)c6)CC4)C5)cc23)C1]

The title compound was prepared in a manner similar to that in Example 6 with intermediate 6. ¹H NMR (500 MHz, DMSO) δ 12.32 (s, 1H), 10.86 (s, 1H), 8.66 (s, 1H), 7.89 (s, 1H), 7.46 (s, 1H), 7.28 (t, J=15.4 Hz, 1H), 6.90 (s, 1H), 6.77 (d, J=8.8 Hz, 1H), 6.60 (d, J=12.8 Hz, 2H), 4.88-4.39 (m, 2H), 4.30 (s, 2H), 4.23-4.08 (m, 1H), 4.03 (dd, J=12.6, 4.9 Hz, 1H), 3.72 (s, 3H), 3.67 (d, J=12.1 Hz, 2H), 2.93-2.89 (m, 3H), 2.77-2.74 (m, 3H), 2.65-2.51 (m, 12H), 2.26 (d, J=3.3 Hz, 1H), 2.13-2.01 (m, 1H), 1.95-1.92 (m, 1H), 1.89-1.77 (m, 4H), 1.74-1.71 (m, 2H), 1.42-1.39 (m, 2H). [M+H]⁺=845.5.

### Example 129: (3R)-3-(4-(4-(7-((7¹s,7³s,E)-1¹,2⁶-dimethyl-3-oxo-5²,5³-dihydro-1¹H,5¹H-9-oxa-4-aza- 5(2,1)-benzo[d]imidazola-2(2,4)-pyridina-1(4,5)-pyrazola-7(1,3)-cyclobutanacyclononaphane-5⁶-yl)-2,7-diazaspiro[4.4]nonan-2-yl)piperidin-1-yl)-2,6-difluorophenyl)piperidine-2,6-dione

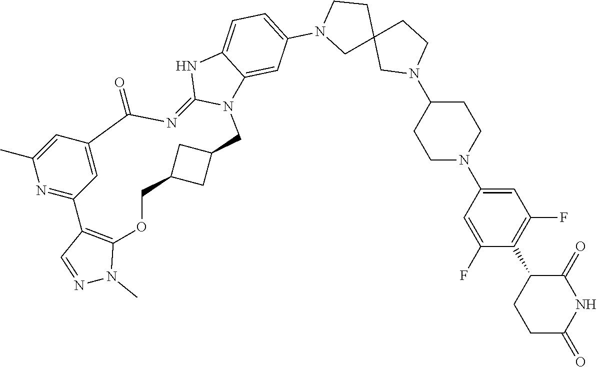 [SMILES: Cc1cc2cc(n1)-c1cnn(C)c1OC[C@H]1C[C@@H](CN3/C(=N/C2=O)Nc2ccc(N4CCC5(CCN(C6CCN(c7cc(F)c([C@H]8CCC(=O)NC8=O)c(F)c7)CC6)C5)C4)cc23)C1]

The titled compound was prepared in a manner similar to that in Example 6 with intermediate 6. ¹H NMR (500 MHz, DMSO) δ 12.31 (s, 1H), 10.87 (s, 1H), 8.65 (s, 1H), 7.89 (s, 1H), 7.45 (s, 1H), 7.31 (d, J=8.4 Hz, 1H), 6.63 (d, J=10.6 Hz, 2H), 6.51 (s, 1H), 6.46 (d, J=8.5 Hz, 1H), 4.62-4.51 (m, 2H), 4.25-4.15 (m, 1H), 4.05 (dd, J=12.6, 4.8 Hz, 1H), 3.71 (s, 4H), 3.68-3.64 (m, 1H), 3.32 (s, 2H), 3.30-3.27 (m, 2H), 3.20 (s, 1H), 2.92 (s, 3H), 2.87-2.79 (m, 4H), 2.69-2.57 (m, 3H), 2.55 (m, 3H), 2.53-2.51 (m, 3H), 2.22 (s, 1H), 2.13-2.02 (m, 2H), 2.01-1.92 (m, 3H), 1.87-1.81 (m, 4H), 1.44 (s, 2H). [M+H]⁺=859.7.

### Example 130: 3-(5-(4-(8-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3,8-diazabicyclo[3.2.1]octan-3-yl)piperidin-1-yl)-4-methylpyridin-2-yl)piperidine-2,6-dione

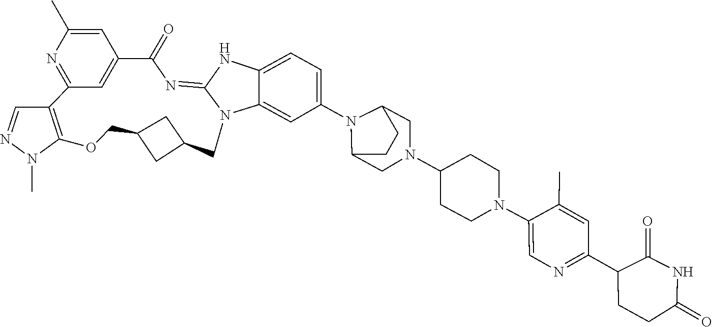 [SMILES: Cc1cc2cc(n1)-c1cnn(C)c1OC[C@H]1C[C@@H](CN3/C(=N/C2=O)Nc2ccc(N4C5CCC4CN(C4CCN(c6cnc(C7CCC(=O)NC7=O)cc6C)CC4)C5)cc23)C1]

The title compound was prepared in a manner similar to that in Example 6 with intermediate 10. ¹H NMR (500 MHz, DMSO) δ 12.32 (s, 1H), 10.80 (s, 1H), 8.66 (s, 1H), 8.11 (s, 1H), 7.89 (s, 1H), 7.46 (s, 1H), 7.30 (d, J=8.7 Hz, 1H), 7.12 (s, 1H), 6.92 (s, 1H), 6.79 (d, J=8.8 Hz, 1H), 4.58 (s, 1H), 4.33 (s, 2H), 3.87 (dd, J=8.7, 5.4 Hz, 1H), 3.72 (s, 3H), 3.12 (d, J=11.6 Hz, 2H), 2.91 (s, 3H), 2.67-2.64 (m, 5H), 2.58 (s, 2H), 2.57-2.54 (m, 8H), 2.36 (s, 1H), 2.24-2.20 (m, 6H), 2.10-2.07 (m, 1H), 1.90 (d, J=6.8 Hz, 2H), 1.81 (d, J=14.4 Hz, 4H), 1.52 (d, J=11.2 Hz, 2H). [M+H]⁺=824.0.

### Example 131: 3-(5-(4-(3-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)azetidin-1-yl)piperidin-1-yl)-4-methylpyridin-2-yl)piperidine-2,6-dione

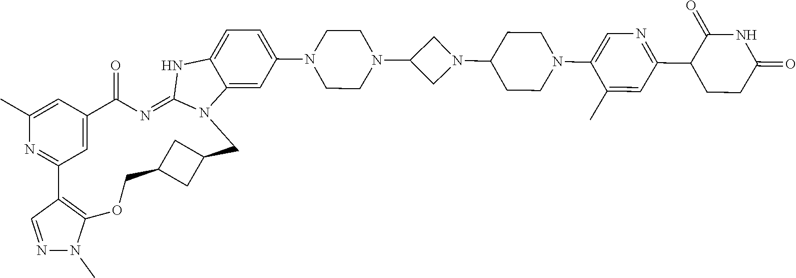 [SMILES: Cc1cc2cc(n1)-c1cnn(C)c1OC[C@H]1C[C@@H](CN3/C(=N/C2=O)Nc2ccc(N4CCN(C5CN(C6CCN(c7cnc(C8CCC(=O)NC8=O)cc7C)CC6)C5)CC4)cc23)C1]

The titled compound was prepared in a manner similar to that in Example 6 with intermediates 39 and 10. ¹H NMR (500 MHz, DMSO) δ 12.40 (s, 1H), 10.79 (s, 1H), 8.65 (s, 1H), 8.13 (s, 1H), 7.89 (s, 1H), 7.46 (s, 1H), 7.35 (d, J=8.7 Hz, 1H), 7.13 (s, 1H), 7.02 (s, 1H), 6.90 (d, J=8.4 Hz, 1H), 4.72-4.62 (m, 2H), 4.32-4.18 (m, 2H), 3.88 (dd, J=8.7, 5.4 Hz, 1H), 3.72 (s, 3H), 3.43 (s, 3H), 3.17 (s, 4H), 3.12-3.06 (m, 2H), 2.92 (s, 5H), 2.71-2.67 (m, 2H), 2.59-2.53 (m, 5H), 2.52-2.51 (m, 1H), 2.44 (s, 5H), 2.23 (s, 4H), 2.20-2.06 (m, 3H), 1.79-1.76 (m, 2H), 1.37-1.31 (m, 2H). [M+H]⁺=853.8.

### Example 132: 3-(5-(4-(5-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2,5-diazabicyclo[2.2.2]octan-2-yl)piperidin-1-yl)-4-methylpyridin-2-yl)piperidine-2,6-dione

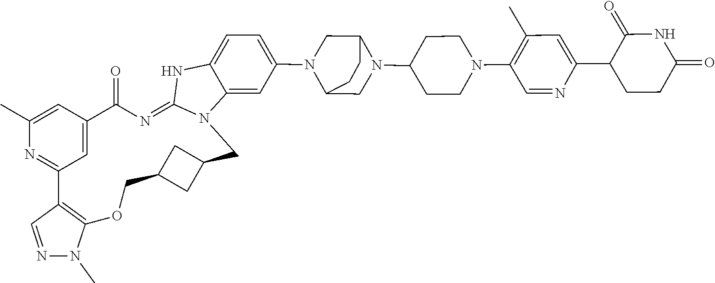 [SMILES: Cc1cc2cc(n1)-c1cnn(C)c1OC[C@H]1C[C@@H](CN3/C(=N/C2=O)Nc2ccc(N4CC5CCC4CN5C4CCN(c5cnc(C6CCC(=O)NC6=O)cc5C)CC4)cc23)C1]

The title compound was prepared in a manner similar to that in Example 6 with intermediate 10. ¹H NMR (500 MHz, DMSO) δ 12.30 (s, 1H), 10.79 (s, 1H), 8.65 (s, 1H), 8.13 (s, 1H), 7.89 (s, 1H), 7.45 (s, 1H), 7.31 (d, J=8.7 Hz, 1H), 7.13 (s, 1H), 6.68 (s, 1H), 6.60 (d, J=8.1 Hz, 1H), 4.84-4.44 (m, 2H), 4.38-4.06 (m, 2H), 4.02 (s, 1H), 3.88 (dd, J=8.7, 5.3 Hz, 1H), 3.71 (s, 3H), 3.61 (d, J=9.2 Hz, 1H), 3.30-3.20 (m, 4H), 3.13 (d, J=6.3 Hz, 2H), 3.04-2.85 (m, 5H), 2.72 (t, J=8.7 Hz, 2H), 2.64-2.53 (m, 6H), 2.28 (s, 1H), 2.24 (s, 3H), 2.21-2.14 (m, 1H), 2.09 (dd, J=12.9, 6.3 Hz, 1H), 2.01 (d, J=11.3 Hz, 1H), 1.99-1.91 (m, 2H), 1.87 (d, J=10.6 Hz, 1H), 1.83-1.75 (m, 11H), 1.65-1.55 (m, 1H), 1.55-1.42 (m, 2H). [M+H]⁺=824.6.

### Example 133: 3-(5-((3S,4R)-4-(4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-4-methylpyridin 2-yl)piperidine-2,6-dione

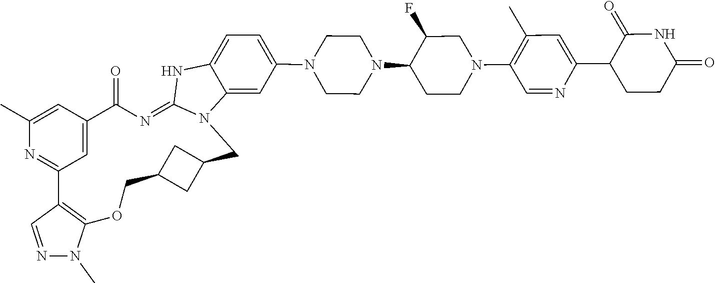 [SMILES: Cc1cc2cc(n1)-c1cnn(C)c1OC[C@H]1C[C@@H](CN3/C(=N/C2=O)Nc2ccc(N4CCN([C@@H]5CCN(c6cnc(C7CCC(=O)NC7=O)cc6C)C[C@@H]5F)CC4)cc23)C1]

The title compound was prepared in a manner similar to that in Example 26 step 4 with intermediates 1 and 32. ¹H NMR (500 MHz, DMSO) δ 12.54 (s, 1H), 10.87 (s, 1H), 8.76 (s, 1H), 8.25 (s, 1H), 8.00 (s, 1H), 7.59 (s, 1H), 7.42 (d, J=8.7 Hz, 1H), 7.28 (s, 1H), 7.18 (s, 1H), 7.00 (d, J=8.9 Hz, 1H), 5.51-5.41 (m, 1H), 4.83-4.52 (m, 2H), 4.30-4.04 (m, 2H), 4.02-3.88 (m, 3H), 3.74 (s, 5H), 3.67-3.61 (m, 1H), 3.42-3.28 (m, 3H), 3.16-3.07 (m, 2H), 3.03-2.98 (m, 1H), 2.98-2.90 (m, 3H), 2.66-2.51 (m, 8H), 2.33 (s, 3H), 2.30-2.28 (m, 1H), 2.25-2.20 (m, 2H), 2.15-2.09 (m, 2H), 1.91-1.68 (m, 1H). [M+H]⁺=816.9.

### Example 134: 3-(5-((3R,4S)-4-(4-((7¹s,7³R,E)-1¹,2⁶-dimethyl-3-oxo-5²,5³-dihydro-1¹H,5′H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-4-methylpyridin-2-yl)piperidine-2,6-dione

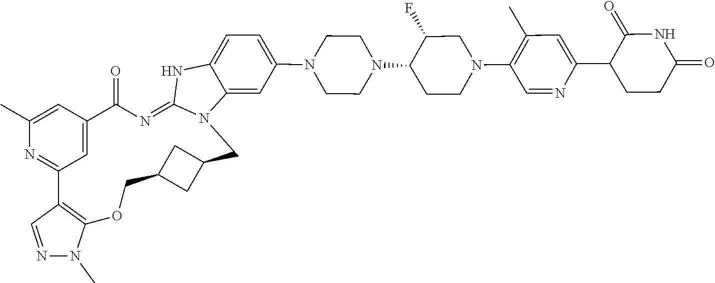 [SMILES: Cc1cc2cc(n1)-c1cnn(C)c1OC[C@H]1C[C@@H](CN3/C(=N/C2=O)Nc2ccc(N4CCN([C@H]5CCN(c6cnc(C7CCC(=O)NC7=O)cc6C)C[C@H]5F)CC4)cc23)C1]

The title compound was prepared in a manner similar to that in Example 26 step 4 with intermediates 1 and 33. ¹H NMR (500 MHz, DMSO) δ 12.55 (s, 1H), 10.88 (s, 1H), 8.76 (s, 1H), 8.25 (s, 1H), 8.00 (s, 1H), 7.59 (s, 1H), 7.42 (d, J=8.7 Hz, 1H), 7.28 (s, 1H), 7.18 (s, 1H), 7.00 (d, J=8.4 Hz, 1H), 5.51-5.42 (m, 1H), 4.87-4.49 (m, 2H), 4.42-4.06 (m, 2H), 4.03-3.87 (m, 3H), 3.78-3.68 (m, 5H), 3.64 (t, J=11.3 Hz, 1H), 3.46-3.25 (m, 3H), 3.18-3.07 (m, 2H), 3.05-2.85 (m, 4H), 2.67-2.51 (m, 8H), 2.38-2.18 (m, 6H), 2.17-2.05 (m, 2H), 1.92-1.58 (m, 1H). [M+H]⁺=816.9.

### Example 66: (R)-3-(4-((3S,4R)-4-(4-((7¹s,7³S,E)-1¹,2⁶-dimethyl-3-oxo-5²,5³-dihydro-1¹H,5¹H-9-oxa- 4-aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-2,6-difluorophenyl)piperidine-2,6-dione

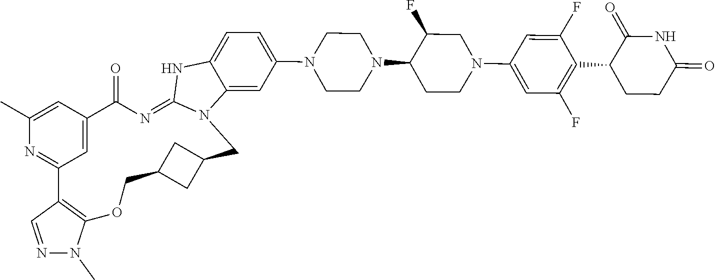 [SMILES: Cc1cc2cc(n1)-c1cnn(C)c1OC[C@H]1C[C@@H](CN3/C(=N/C2=O)Nc2ccc(N4CCN([C@@H]5CCN(c6cc(F)c([C@H]7CCC(=O)NC7=O)c(F)c6)C[C@@H]5F)CC4)cc23)C1]

The title compound was prepared in a manner similar to that in Example 26 step 4 with intermediates 1 and 34, and purified by silica gel column (DCM:CH₃OH=15:1) to give the racemate, which was further purified by Prep chiral-HPLC with following condition: (Column: CHIRALPAK IF, 2 cm×25 cm, 5 um; Flow rate: 20 mL/min; Gradient: 10% to 50% in 2.0 min, hold 1.0 min at 50%, Injection Volume: 5 uL) to afford the title product. ¹H NMR (500 MHz, DMSO) δ 12.38 (s, 1H), 10.83 (s, 1H), 8.66 (s, 1H), 7.89 (s, 1H), 7.46 (s, 1H), 7.35 (d, J=8.6 Hz, 1H), 7.01 (s, 1H), 6.90 (d, J=8.3 Hz, 1H), 6.64 (d, J=13.0 Hz, 2H), 5.19-5.04 (m, 1H), 4.85-4.01 (m, 6H), 3.92 (d, J=12.5 Hz, 1H), 3.71 (s, 3H), 3.21-3.12 (m, 4H), 3.05-2.73 (m, 10H), 2.70-2.52 (m, 8H), 2.14-2.04 (m, 1H), 2.00-1.85 (m, 2H), 1.79 (s, 1H). [M+H]⁺=837.7.

### Example 136: (R)-3-(4-(4-(6-(((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)(methyl)amino)-2-azaspiro[3.3]heptan-2-yl)piperidin-1-yl)-2,6-difluorophenyl)piperidine-2,6-dione

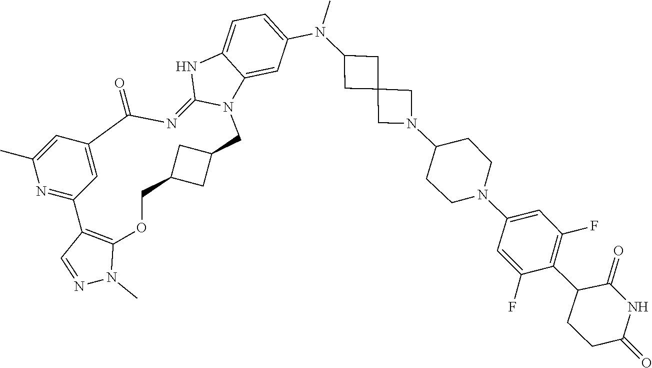 [SMILES: Cc1cc2cc(n1)-c1cnn(C)c1OC[C@H]1C[C@@H](CN3/C(=N/C2=O)Nc2ccc(N(C)C4CC5(C4)CN(C4CCN(c6cc(F)c(C7CCC(=O)NC7=O)c(F)c6)CC4)C5)cc23)C1]

The title compound was prepared in a manner similar to that in Example 6 with intermediate 6. ¹H NMR (500 MHz, DMSO) δ 12.37 (s, 1H), 10.87 (s, in), 8.65 (s, 1H), 7.90 (s, 1H), 7.46 (s, 1H), 7.33 (d, J=8.6 Hz, 1H1), 6.80-6.67 (m, 2H), 6.59 (d, J=12.9 Hz, 2H), 4.86-3.96 (m, 6H), 3.82-3.75 (in, i1H), 3.71 (s, 3H), 3.63-3.51 (m, 3H), 3.21 (s, 2H), 3.04 (s, 2H), 2.93-2.87 (m, 2H), 2.81-2.73 (m, 6H), 2.57-2.52 (m, 4H), 2.44-2.35 (m, 3H), 2.13-2.03 (m, 4H), 1.96-1.89 (m, 2H), 1.67-1.59 (m, 2H), 1.18-1.13 (in, 2H). [M+H]⁺=859.70.

### Example 137: (R)-3-(4-(4-(3-(4-((7¹s,7³s,E)-1¹,2⁶-dimethyl-3-oxo-5²,5³-dihydro-1¹H,5¹H-9-oxa-4- aza-5(2,1)-benzo[d]imidazola-2(2,4)-pyridina-1(4,5)-pyrazola-7(1,3)-cyclobutanacyclononaphane-5⁶-yl)piperazin-1-yl)azetidin-1-yl)piperidin-1-yl)-2,6-difluorophenyl)piperidine-2,6-dione

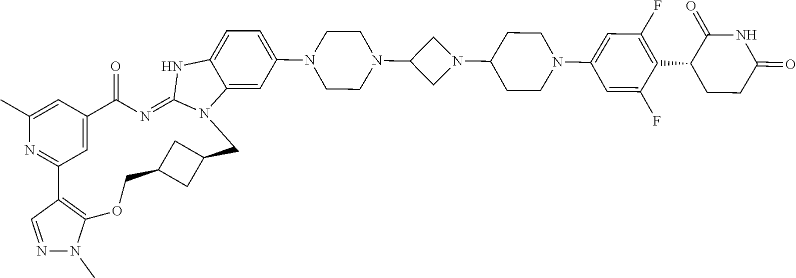 [SMILES: Cc1cc2cc(n1)-c1cnn(C)c1OC[C@H]1C[C@@H](CN3/C(=N/C2=O)Nc2ccc(N4CCN(C5CN(C6CCN(c7cc(F)c([C@H]8CCC(=O)NC8=O)c(F)c7)CC6)C5)CC4)cc23)C1]

The title compound was prepared in a manner similar to that in Example 6 with intermediates 39 and 6. ¹H NMR (500 MHz, DMSO) δ 12.41 (s, 1H), 10.88 (s, 1H), 8.65 (s, 1H), 7.90 (s, 1H), 7.46 (s, 1H), 7.35 (d, J=8.7 Hz, 1H), 6.99 (s, 1H), 6.87 (d, J=8.4 Hz, 1H), 6.59 (d, J=12.9 Hz, 2H), 4.87-3.96 (m, 6H), 3.71 (s, 3H), 3.59-3.51 (m, 2H), 3.37 (s, 3H), 3.13 (s, 4H), 2.96-2.74 (m, 9H), 2.58-2.52 (m, 4H), 2.39 (s, 4H), 2.17-2.06 (m, 2H), 1.98-1.94 (m, 2H), 1.68-1.63 (m, 2H), 1.24-1.13 (m, 2H). [M+H]⁺=874.70.

### Example 138: 3-(5-((3R,4R)-4-(4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)-3-fluoropiperidin-1-yl)-4-methylpyridin-2-yl)piperidine-2,6-dione

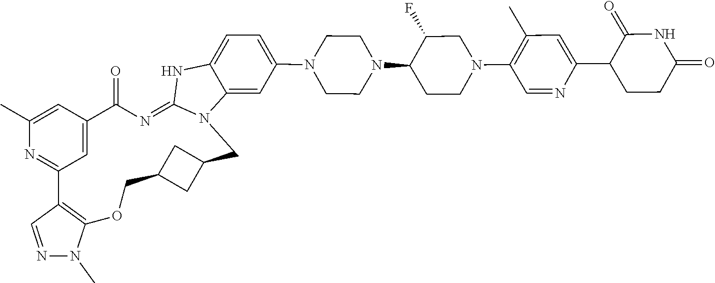 [SMILES: Cc1cc2cc(n1)-c1cnn(C)c1OC[C@H]1C[C@@H](CN3/C(=N/C2=O)Nc2ccc(N4CCN([C@@H]5CCN(c6cnc(C7CCC(=O)NC7=O)cc6C)C[C@H]5F)CC4)cc23)C1]

The title compound was prepared in a manner similar to that in Example 26 step 4 with intermediate 35. ¹H NMR (500 MHz, DMSO) δ 12.57 (s, 1H), 10.90 (s, 1H), 8.81 (s, 1H), 8.30 (s, 1H), 8.06 (s, 1H), 7.65 (s, 1H), 7.43 (d, J=8.7 Hz, 1H), 7.32 (s, 1H), 7.18 (s, 1H), 7.01 (d, J=8.7 Hz, 1H), 5.53-5.21 (m, 1H), 4.78-4.57 (m, 2H), 4.29-4.24 (m, 2H), 4.01 (dd, J=9.6, 5.2 Hz, 3H), 3.87-3.81 (m, 2H), 3.75 (s, 3H), 3.69-3.66 (m, 1H), 3.51-3.43 (m, 1H), 3.28 (d, J=10.4 Hz, 2H), 3.06-2.76 (m, 6H), 2.72-2.51 (m, 8H), 2.37-2.21 (m, 6H), 2.16-2.08 (m, 1H), 2.04-1.90 (m, 1H), 1.89-1.61 (m, 1H). [M+H]⁺=816.7.

### Example 139: 3-(5-(4-((2S,5S)-4-((7¹s,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-2,5-dimethylpiperazin-1-yl)piperidin-1-yl)-6-methylpyridin-2-yl)piperidine-2,6-dione

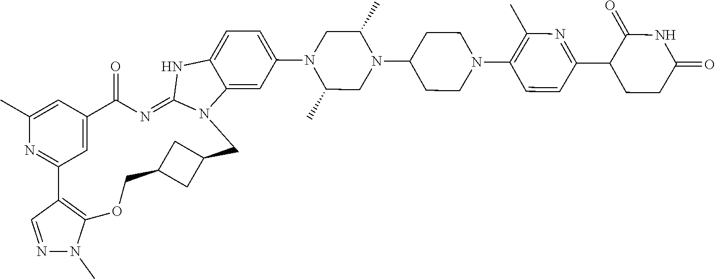 [SMILES: Cc1cc2cc(n1)-c1cnn(C)c1OC[C@H]1C[C@@H](CN3/C(=N/C2=O)Nc2ccc(N4C[C@H](C)N(C5CCN(c6ccc(C7CCC(=O)NC7=O)nc6C)CC5)C[C@@H]4C)cc23)C1]

The title compound was prepared in a manner similar to that in Example 6 with intermediates 1 and 12. ¹H NMR (500 MHz, DMSO) δ 12.51 (s, 1H), 10.84 (s, 1H), 8.73 (s, 1H), 7.97 (s, 1H), 7.55 (s, 1H), 7.50 (d, J=8.3 Hz, 1H), 7.41 (d, J=8.7 Hz, 1H), 7.19 (s, 1H), 7.09 (s, 1H), 7.00-6.93 (m, 1H), 3.81-3.71 (m, 6H), 3.69-3.62 (m, 3H), 3.29-3.24 (m, 5H), 3.18-3.00 (m, 3H), 3.00-2.80 (m, 5H), 2.77-2.71 (m, 1H), 2.60 (s, 6H), 2.41 (s, 2H), 2.36-2.32 (m, 1H), 2.27-2.20 (m, 2H), 2.15-2.04 (m, 3H), 1.93-1.71 (m, 2H), 1.55-1.48 (m, 3H), 1.12 (d, J=6.8 Hz, 2H), 0.96 (d, J=5.9 Hz, 1H). [M+H]⁺=826.7.

### Example 140: 3-(5-(4-(4-((7¹s,7³s,E)-1¹,2⁶-dimethyl-3-oxo-5²,5³-dihydro-1H,5¹H-9-oxa-4-aza-5(2,1)- benzo[d]imidazola-2(2,4)-pyridina-1(4,5)-pyrazola-7(1,3)-cyclobutanacyclononaphane-5⁶-yl)piperazin-1-yl)piperidin-1-yl)-6-ethylpyridin-2-yl)piperidine-2,6-dione

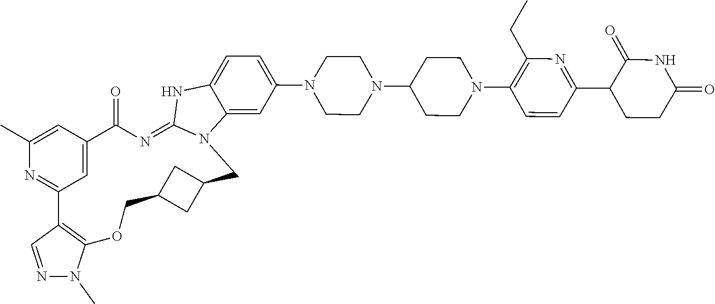 [SMILES: CCc1nc(C2CCC(=O)NC2=O)ccc1N1CCC(N2CCN(c3ccc4c(c3)N3C[C@H]5C[C@@H](COc6c(cnn6C)-c6cc(cc(C)n6)C(=O)/N=C/3N4)C5)CC2)CC1]

The title compound was prepared in a manner similar to that in Example 6 with intermediate 36. ¹H NMR (500 MHz, DMSO) δ 12.41 (s, 1H), 10.79 (s, 1H), 8.66 (s, 1H), 7.89 (s, 1H), 7.46 (s, 1H), 7.41 (d, J=8.2 Hz, 1H), 7.35 (d, J=8.7 Hz, 1H), 7.12 (d, J=8.2 Hz, 1H), 7.02 (s, 1H), 6.91 (dd, J=8.8, 1.7 Hz, 1H), 4.85-3.99 (m, 4H), 3.91 (dd, J=7.7, 5.6 Hz, 1H), 3.72 (s, 3H), 3.20-3.14 (m, 4H), 3.08 (s, 2H), 2.96-2.88 (m, 2H), 2.84-2.52 (m, 16H), 2.38 (d, J=12.0 Hz, 2H), 2.21-2.12 (m, 2H), 1.92 (d, J=11.6 Hz, 2H), 1.63 (d, J=11.5 Hz, 2H), 1.18 (t, J=7.5 Hz, 3H). [M+H]⁺=812.6.

### Example 141: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)piperidin-1-yl)-4,6-dimethylpyridin-2-yl)piperidine-2,6-dione

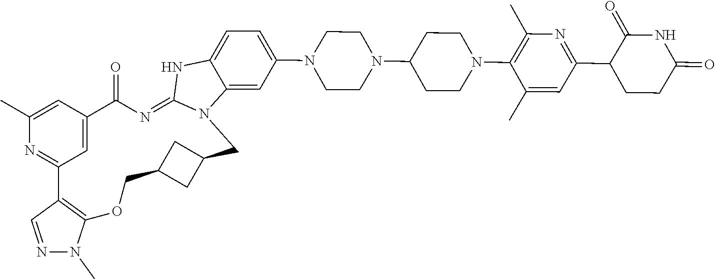 [SMILES: Cc1cc2cc(n1)-c1cnn(C)c1OC[C@H]1C[C@@H](CN3/C(=N/C2=O)Nc2ccc(N4CCN(C5CCN(c6c(C)cc(C7CCC(=O)NC7=O)nc6C)CC5)CC4)cc23)C1]

The title compound was prepared in a manner similar to that in Example 6 with intermediate 37. ¹H NMR (500 MHz, DMSO) δ 12.41 (s, 1H), 10.78 (s, 1H), 8.66 (s, 1H), 7.89 (s, 1H), 7.46 (s, 1H), 7.35 (d, J=8.7 Hz, 1H), 7.03-6.88 (m, 3H), 4.85-3.99 (m, 4H), 3.84 (dd, J=9.2, 5.4 Hz, 1H), 3.72 (s, 3H), 3.20-3.08 (m, 6H), 3.01-2.85 (m, 5H), 2.75-2.66 (m, 4H), 2.65-2.51 (m, 7H), 2.45-2.35 (m, 5H), 2.28 (s, 3H), 2.19 (dd, J=8.9, 5.4 Hz, 1H), 2.08 (d, J=6.1 Hz, 1H), 1.86 (d, J=11.0 Hz, 2H), 1.58 (d, J=8.1 Hz, 2H). [M+H]⁺=812.6.

### Example 142: 3-(5-(4-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)piperidin-1-yl)-3-fluoro-6-methylpyridin-2-yl)piperidine-2,6-dione

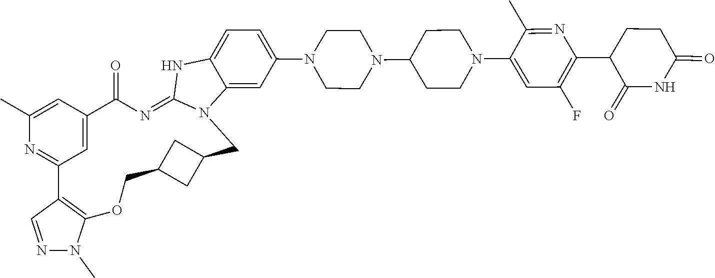 [SMILES: Cc1cc2cc(n1)-c1cnn(C)c1OC[C@H]1C[C@@H](CN3/C(=N/C2=O)Nc2ccc(N4CCN(C5CCN(c6cc(F)c(C7CCC(=O)NC7=O)nc6C)CC5)CC4)cc23)C1]

The title compound was prepared in a manner similar to that in Example 6 with intermediate 38. ¹H NMR (500 MHz, DMSO) δ 12.39 (s, 1H), 10.84 (s, 1H), 8.66 (s, 1H), 7.89 (s, 1H), 7.46 (s, 1H), 7.38-7.29 (m, 2H), 7.01 (s, 1H), 6.90 (d, J=8.7 Hz, 1H), 4.90-4.08 (m, 6H), 3.72 (s, 3H), 3.17 (s, 6H), 2.96-2.86 (m, 2H), 2.75-2.63 (m, 9H), 2.59-2.53 (m, 6H), 2.38 (s, 3H), 2.28-2.23 (m, 1H), 2.07-2.02 (m, 1H), 1.97-1.90 (m, 2H), 1.67-1.58 (m, 2H).

[M+H]⁺=816.6.

### Example 143: 3-(5-(4-(3-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)azetidin-1-yl)piperidin-1-yl)-4-ethylpyridin-2-yl)piperidine-2,6-dione

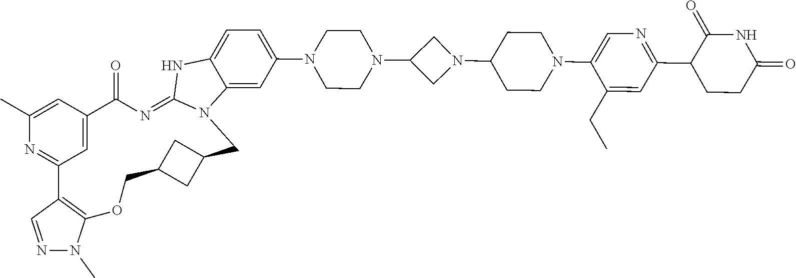 [SMILES: CCc1cc(C2CCC(=O)NC2=O)ncc1N1CCC(N2CC(N3CCN(c4ccc5c(c4)N4C[C@H]6C[C@@H](COc7c(cnn7C)-c7cc(cc(C)n7)C(=O)/N=C/4N5)C6)CC3)C2)CC1]

The title compound was prepared in a manner similar to that in Example 6 with intermediate 39. ¹H NMR (500 MHz, DMSO) δ 12.41 (s, 1H), 10.80 (s, 1H), 8.65 (s, 1H), 8.17 (s, 1H), 7.89 (s, 1H), 7.46 (s, 1H), 7.35 (d, J=8.7 Hz, 1H), 7.18 (s, 1H), 7.02 (s, 1H), 6.90 (d, J=8.9 Hz, 1H), 4.85-4.47 (m, 2H), 4.32-4.01 (m, 2H), 3.94-3.91 (m, 1H), 3.72 (s, 3H), 3.42-3.40 (m, 2H), 3.16 (s, 3H), 3.07-2.99 (m, 3H), 2.95-2.90 (m, 4H), 2.84 (t, J=6.3 Hz, 2H), 2.73-2.68 (m, 3H), 2.65-2.58 (m, 4H), 2.56 (s, 4H), 2.43 (s, 4H), 2.25-2.06 (m, 4H), 1.77-1.75 (m, 2H), 1.34-1.32 (m, 2H), 1.20 (t, J=7.5 Hz, 3H). [M+H]⁺=867.7.

### Example 148: (R)-3-(4-(4-(3-((R)-4-((7¹s,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)-3-methylpiperazin-1-yl)azetidin-1-yl)piperidin-1-yl)-2,6-difluorophenyl)piperidine-2,6-dione

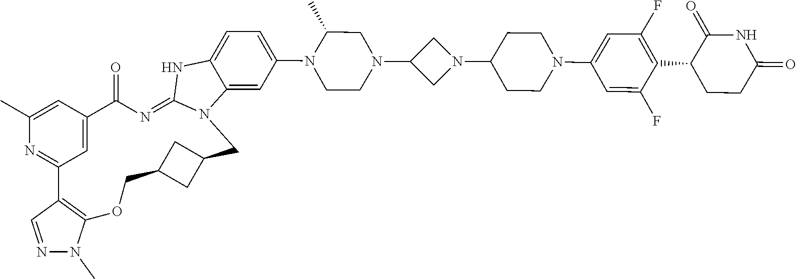 [SMILES: Cc1cc2cc(n1)-c1cnn(C)c1OC[C@H]1C[C@@H](CN3/C(=N/C2=O)Nc2ccc(N4CCN(C5CN(C6CCN(c7cc(F)c([C@H]8CCC(=O)NC8=O)c(F)c7)CC6)C5)C[C@H]4C)cc23)C1]

The title compound was prepared in a manner similar to that in Example 6. ¹H NMR (500 MHz, DMSO) δ 12.41 (s, 1H), 10.86 (s, 1H), 8.65 (s, 1H), 7.89 (s, 1H), 7.46 (s, 1H), 7.35 (d, J=8.7 Hz, 1H), 7.01 (s, 1H), 6.88 (d, J=9.1 Hz, 1H), 6.63 (s, 1H), 6.60 (s, 1H), 4.93-4.44 (m, 2H), 4.39-4.09 (m, 2H), 4.04 (dd, J=12.4, 4.8 Hz, 1H), 3.90 (d, J=5.2 Hz, 1H), 3.72 (s, 3H), 3.60 (d, J=12.4 Hz, 2H), 3.46-3.36 (m, 4H), 3.23-3.18 (m, 1H), 2.98 (t, J=9.0 Hz, 1H), 2.95-2.82 (m, 7H), 2.82-2.74 (m, 2H), 2.67-2.61 (m, 1H), 2.59-2.51 (m, 5H), 2.44 (d, J=8.8 Hz, 1H), 2.33-2.28 (m, 1H), 2.22 (ddd, J=13.1, 8.4, 4.1 Hz, 1H), 2.17-2.11 (m, 1H), 2.10-2.03 (m, 1H), 1.99-1.91 (m, 1H), 1.70 (d, J=10.0 Hz, 2H), 1.21 (d, J=9.4 Hz, 2H), 0.96 (d, J=6.3 Hz, 3H). [M+H]⁺=888.7.

### Example 149: 3-(5-(4-(3-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)azetidin-1-yl)piperidin-1-yl)-6-ethylpyridin-2-yl)piperidine-2,6-dione

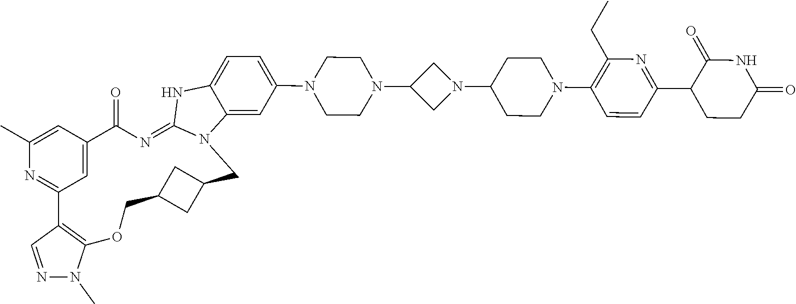 [SMILES: CCc1nc(C2CCC(=O)NC2=O)ccc1N1CCC(N2CC(N3CCN(c4ccc5c(c4)N4C[C@H]6C[C@@H](COc7c(cnn7C)-c7cc(cc(C)n7)C(=O)/N=C/4N5)C6)CC3)C2)CC1]

The title compound was prepared in a manner similar to that in Example 6 with intermediates 39 and 36. ¹H NMR (500 MHz, DMSO) δ 12.41 (s, 1H), 10.78 (s, 1H), 8.65 (s, 1H), 7.89 (s, 1H), 7.46 (s, 1H), 7.39 (d, J=8.2 Hz, 1H), 7.35 (d, J=8.7 Hz, 1H), 7.11 (d, J=8.2 Hz, 1H), 7.02 (s, 1H), 6.90 (d, J=8.8 Hz, 1H), 4.87-3.99 (m, 4H), 3.90 (dd, J=7.6, 5.7 Hz, 1H), 3.72 (s, 3H), 3.41 (t, J=6.0 Hz, 3H), 3.18-3.13 (m, 4H), 3.01-2.82 (m, 8H), 2.80-2.53 (m, 11H), 2.46-2.39 (m, 4H), 2.15 (dd, J=12.1, 6.3 Hz, 3H), 1.76 (d, J=11.5 Hz, 2H), 1.35 (d, J=9.0 Hz, 2H), 1.17 (t, J=7.4 Hz, 3H). [M+H]⁺=867.6.

### Example 150: 3-(5-(2-(3-(4-((7¹s,7³s,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclobutanacyclononaphane-5⁶-yl)piperazin-1-yl)azetidin-1-yl)ethyl)-4,6-dimethylpyridin-2-yl)piperidine-2,6-dione

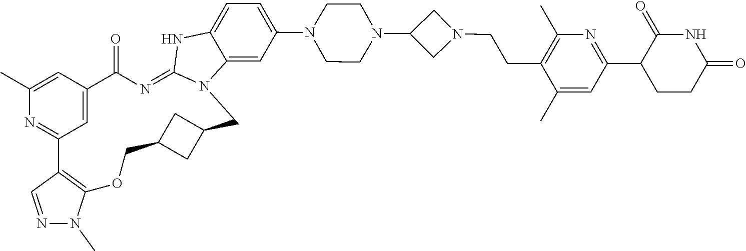 [SMILES: Cc1cc2cc(n1)-c1cnn(C)c1OC[C@H]1C[C@@H](CN3/C(=N/C2=O)Nc2ccc(N4CCN(C5CN(CCc6c(C)cc(C7CCC(=O)NC7=O)nc6C)C5)CC4)cc23)C1]

The title compound was prepared in a manner similar to that in Example 82 with intermediates 39 and 26.

¹H NMR (500 MHz, DMSO) δ 12.42 (s, 1H), 10.80 (s, 1H), 8.65 (s, 1H), 7.89 (s, 1H), 7.46 (s, 1H), 7.34 (d, J=8.7 Hz, 1H), 7.02 (s, 1H), 6.97 (s, 1H), 6.90 (d, J=8.7 Hz, 1H), 4.80-3.82 (m, 6H), 3.72 (s, 3H), 3.51-3.45 (m, 2H), 3.16 (s, 4H), 2.98-2.83 (m, 6H), 2.65-2.52 (m, 10H), 2.46-2.39 (m, 7H), 2.29 (s, 3H), 2.21-2.19 (m, 1H), 2.09-2.04 (m, 11H), 1.84-1.61 (m, 11H). [M+H]⁺=812.6.

### Cell Line Generation

H1975-clone #28 (Del19/T790M/C797S, or abbr: DTC), H1975-clone #23 (Del19/C797S, or abbr: DC), H1975-clone #25 (L858R/T790M/C797S, or abbr: LTC) and H1975-clone #8 (L858R/C₇₉₇S, or abbr: LC). EGFR-Del19/T790M/C797S, EGFR-Del19/C797S, EGFR-L858R/T790M/C797S and EGFR-L858R/C797S were stably expressed in H1975 cell lines by lentivirus-mediated over-expression, respectively. The EGFR over-expressed cells then underwent knockout, in which the EGFR targeting sgRNA was designed to only target the endogenous EGFR copies and preserve the exogenous EGFR copies. Followed by the knockout, the edited H1975 cells were seeded in 96 well plates at the concentration of 1 cell/well, cultured for about 2 weeks to allow single clones formation. The formed clones were screened by DNA sequencing and whole exon sequencing analysis for the desired edition. H1975-clone #28, H1975-clone #23, H1975-clone #25 and H1975-clone #8 were finally confirmed as homozygous Del19/T790M/C₇₉₇S EGFR, Del19/C₇₉₇S EGFR, L858R/T790M/C₇₉₇S EGFR and L858R/C₇₉₇S EGFR clones, respectively.

### Cell Degradation

### Cell Treatment

On day 1, H1975-clone #28 (Del19/T790M/C797S, or abbr: DTC), H1975-clone #23 (Del19/C797S, or abbr: DC), H1975-clone #25 (L858R/T790M/C797S, or abbr: LTC) and H1975-clone #8 (L858R/C797S, or abbr: LC) cells are seeded at 20000 cells/well, 30000 cells/well, 10000 cells/well or 5000 cells/well correspondingly in cell culture medium [RPMIl640 (Gibco, Cat #72400-047), 10% heat-inactive FBS, 1% PS (Gibco, Cat #10378)] in Corning 96 well plate (Cat #3599).

H1975-#25, H1975-#28, H1975-#23 and H1975-#8 cells are treated with compounds diluted in 0.2% DMSO cell culture medium on day 2, incubate for 16 h, 37° C., 5% CO₂. The final concentration of compounds in all assay is start with 10 μM, 5-fold dilution, total 8 doses were included.

### HTRF Assay

Total-EGFR cellular kit(64NG1PEH) were used for evaluating EGFR degradation.

After 16 h treatment, add HTRF lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; cover the plate with a plate sealer, spin 1000 rpm for 1 min, incubate overnight at room temperature; read on BMG PheraStar with HTRF protocol (337 nm-665 nm-620 nm).

The inhibition (degradation) percentage of the compound was calculated by the following equation:

Inhibition percentage of Compound=100−100×(Signal−low control)/(High control−low control), wherein signal=each test compound group

Low control=only lysis buffer without cells, indicating that EGFR is completely degraded.

High control=Cell group with added DMSO and without compound, indicating microplate readings without EGFR degradation.

Imax (Dmax) is the maximum percentage of inhibition (degradation).

The IC₅₀ (DC₅₀) value of a compound can be obtained by fitting the following equation

$$Y = Bottom + ( TOP - Bottom ) / ( 1 + ( ( {IC}_{50} / X ) \hat{} hillslope ) )$$

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (H1975 # 28 DTC and H1975
# 25-LTC) result for Examples

| | |
|---|---|
| H1975 # 28 DTC | H1975 # 25-LTC |

| | | | | |
|---|---|---|---|---|
| example 1 | 0.6 | 91.9 | 1.0 | 92.8 |
| example 2 | 0.8 | 84.3 | 3.1 | 62.0 |
| example 3 | 0.6 | 86.7 | 0.6 | 85.6 |
| example 4 | 1.0 | 84.6 | 1.1 | 86.6 |
| example 5 | 0.6 | 92.2 | 0.5 | 92.9 |
| example 6 | 0.8 | 91.2 | 0.6 | 91.7 |
| example 7 | 0.8 | 80.6 | 1.9 | 78.6 |
| example 9 | 0.6 | 90.9 | 1.4 | 88.5 |
| example 10 | 0.5 | 90.6 | 3.7 | 89.8 |
| example 11 | 0.5 | 91.5 | 1.9 | 85.4 |
| example 12 | 7.5 | 85.3 | 15.0 | 79.0 |
| example 14 | 0.6 | 77.1 | 1.0 | 83.9 |
| example 15 | 1.4 | 76.0 | 4.5 | 87.3 |
| example 16 | 0.7 | 71.5 | 1.9 | 64.0 |
| example 17 | 0.7 | 86.2 | 1.3 | 88.3 |
| example 18 | 0.6 | 88.4 | 1.6 | 88.6 |
| example 19 | 0.6 | 92.8 | 0.6 | 92.2 |
| example 20 | 0.6 | 89.8 | 1.2 | 92.5 |
| example 21 | 1.7 | 91.5 | 2.1 | 94.0 |
| example 22 | 0.6 | 89.5 | 0.8 | 91.7 |
| example 23 | 0.6 | 87.0 | 1.0 | 91.3 |
| example 24 | 0.6 | 88.0 | 1.2 | 91.7 |
| example 25 | 0.7 | 89.9 | 0.8 | 83.4 |
| example 26 | 1.0 | 90.8 | 1.1 | 92.1 |
| example 27 | 1.1 | 84.7 | 1.9 | 89.6 |
| example 28 | 0.6 | 79.8 | 0.7 | 92.1 |
| example 29 | 0.6 | 78.3 | 0.9 | 42.6 |
| example 30 | 0.6 | 84.4 | 0.8 | 90.3 |
| example 31 | 0.6 | 84.2 | 0.5 | 90.5 |
| example 32 | 0.6 | 88.8 | 0.4 | 88.8 |
| example 33 | 0.6 | 89.4 | 0.6 | 93.3 |
| example 34 | 0.5 | 89.6 | 0.7 | 93.3 |
| example 35 | 0.9 | 72.9 | 1.1 | 85.9 |
| example 36 | 0.7 | 78.0 | 1.3 | 87.7 |
| example 37 | 0.6 | 79.4 | 0.6 | 80.7 |
| example 38 | 0.9 | 78.4 | 1.5 | 81.4 |
| example 40 | 0.7 | 91.5 | 0.4 | 93.6 |
| example 102 | 0.6 | 92.2 | 0.5 | 92.9 |
| example 70 | 0.6 | 88.7 | 0.6 | 91.8 |
| example 41 | 0.6 | 80.1 | 1.6 | 84.2 |
| example 82 | 0.5 | 79.5 | 0.6 | 90.3 |
| example 62 | 0.6 | 89.1 | 0.6 | 91.5 |
| example 43 | 0.5 | 75.5 | 0.6 | 87.1 |
| example 109 | 0.8 | 81.0 | 0.9 | 90.4 |
| example 110 | 0.6 | 82.2 | 0.9 | 90.3 |
| example 57 | 0.3 | 87.5 | 0.6 | 89.9 |
| example 112 | 0.7 | 75.2 | 0.7 | 85.2 |
| example 113 | 0.1 | 75.7 | 1.0 | 78.4 |
| example 114 | 0.8 | 79.0 | 0.8 | 86.8 |
| example 47 | 0.6 | 78.5 | 0.5 | 93.3 |
| example 117 | 0.8 | 78.2 | 0.7 | 90.6 |
| example 44 | 0.6 | 80.7 | 3.0 | 91.3 |
| example 120 | 0.7 | 82.4 | 0.9 | 83.1 |
| example 121 | 0.6 | 83.2 | 0.4 | 89.5 |
| example 122 | 1.3 | 84.1 | 0.9 | 90.9 |
| example 123 | 0.7 | 85.6 | 1.5 | 92.1 |
| example 124 | 1.4 | 82.6 | 1.2 | 87.5 |
| example 125 | 0.7 | 89.9 | 3.0 | 86.8 |
| example 126 | 0.6 | 80.5 | 0.4 | 85.9 |
| example 127 | 0.6 | 85.5 | 1.1 | 96.0 |
| example 128 | 1.0 | 75.7 | 2.0 | 73.7 |
| example 129 | 1.2 | 73.2 | 1.1 | 72.7 |
| example 130 | 0.6 | 83.4 | 1.5 | 90.7 |
| example 131 | 0.5 | 68.0 | 0.3 | 84.8 |
| example 132 | 1.1 | 77.3 | 0.3 | 88.3 |
| example 133 | 0.6 | 88.8 | 0.6 | 92.1 |
| example 134 | 0.5 | 89.6 | 0.5 | 92.0 |
| example 66 | 0.5 | 82.1 | 1.1 | 75.9 |
| example 136 | 1.3 | 85.0 | 2.5 | 87.0 |
| example 137 | 0.3 | 80.2 | 0.6 | 77.3 |
| example 138 | 0.4 | 90.9 | 2.8 | 88.4 |

### Cell Line

H1975-clone #8 (L858R/C797S): EGFR-L858R/C797S were stably expressed in H1975 cell lines by lentivirus-mediated over-expression, respectively. The EGFR over-expressed cells then underwent knockout, in which the EGFR targeting sgRNA was designed to only target the endogenous EGFR copies and preserve the exogenous EGFR copies. Followed by the knockout, the edited H1975 cells were seeded in 96 well plates at the concentration of 1 cell/well, cultured for about 2 weeks to allow single clones formation. The formed clones were screened by DNA sequencing and whole exon sequencing analysis for the desired edition. H1975-clone #8 were finally confirmed as homozygous L858R/C797S EGFR clones.

BaF3-L858R (abbv. L858R) cell were purchased from Kangyuan Bochuang Biotechnology (Beijing) Co., Ltd.

### Cell Degradation

### Cell Treatment

On day 1, H1975-clone #8 (L858R/C797S) cells are seeded at 5000 cells/well in cell culture medium [RPMI1640 (Gibco, Cat #72400-047), 10% heat-inactive FBS, 1% PS (Gibco, Cat #10378)] in Corning 96 well plate (Cat #3599).

On day 2, BaF3-L858R cells are seeded at 50000 cells/well at a volume of 54 μL/well in cell culture medium [RPMI1640 (Gibco, phenol red free, Cat #11835-030), 10% heat-inactive FBS, 1% PS (Gibco, Cat #10378)] in Corning 96 well plate (Cat #3799).

H1975-#8 and BaF3-L858R cells are treated with compounds diluted in 0.1% DMSO cell culture medium on day 2, incubate for 16 h, 37° C., 5% CO₂. the final concentration of compounds in all assay starts with 10 uM, 5-fold dilution, total 8 doses were included.

### HTRF Assay

Total-EGFR cellular kit (64NG1PEH) were used for evaluating EGFR degradation.

After 16 h treatment, for H1975-#8 cells, add 100 μL HTRF 1× lysis buffer to each well; for BaF3-L858R cells, add 20 μL 4× lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; cover the plate with a plate sealer, spin 1000 rpm for 1 min, incubate overnight at room temperature; read on BMG PheraStar with HTRF protocol (337 nm-665 nm-620 nm).

The inhibition (degradation) percentage of the compound was calculated by the following equation:

Inhibition percentage of Compound=100−100×(Signal−low control)/(High control−low control), wherein signal=each test compound group

Low control=only lysis buffer without cells, indicating that EGFR is completely degraded.

High control=Cell group with added DMSO and without compound, indicating microplate readings without EGFR degradation.

Imax (Dmax) is the maximum percentage of inhibition (degradation).

The IC₅₀ (DC₅₀) value of a compound can be obtained by fitting the following equation

$$Y = Bottom + ( TOP - Bottom ) / ( 1 + ( ( {IC}_{50} / X ) \hat{} hillslope ) )$$

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 2
Degradation result for Examples

| | BaF3-L858R | | H1975-clone#8 (L858R/C797S) | |
|---|---|---|---|---|
| Example No | DC50 (nM) | Dmax (%) | DC50 (nM) | Dmax (%) |
| 139 | 30.7 | 88.9 | | |
| 140 | 37.8 | 90.6 | | |
| 141 | 11.9 | 85.1 | 0.7 | 40.4 |
| 142 | 52.3 | 76.2 | | |
| 143 | 8.5 | 75.8 | | |
| 148 | 3.0 | 80.0 | | |
| 149 | 16.0 | 71.1 | 3.3 | 52.2 |
| 150 | 11.9 | 73.5 | 3.5 | 51.9 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

1. A compound of Formula (I): 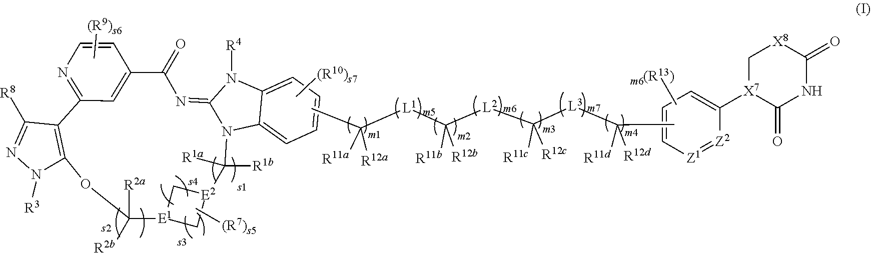
or a pharmaceutically acceptable salt thereof, or a stereoisomer thereof or a tautomer thereof or a deuterated analog thereof,
wherein:
E¹ is N or CR⁵;
E² is N or CR⁶;
R^{1a}, R^{1b}, R^{2a} and R^{2b} are each independently absence, hydrogen, halogen, —C₁₋₈alkyl, —C₂₋₈alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloakyl or —CN;
R³ and R⁴ are each independently hydrogen, —C₁₋₆alkyl, or —C₃₋₈cycloalkyl; each said —C₁₋₆alkyl or —C₃₋₈cycloalkyl is optionally substituted with at least one substituent selected from hydrogen, halogen, —C₁₋₆alkoxy;
R⁵ and R⁶ are each independently absence, hydrogen, halogen, —C₁₋₈alkyl, —C₂₋₈alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl or —CN; or
R⁵ and R⁶ with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;
R⁷ is each independently absence, hydrogen, halogen, —C₁₋₈alkyl, —C₂₋₈alkenyl, —C₂₋₈alkynyl, —C₁₋₈alkoxy, —C₃₋₈cycloalkyl or —CN; each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl or —CN; or
two R⁷ with the carbon atom(s) to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;
R⁸ and R⁹ are each independently selected from hydrogen, halogen, —C₁-C₆alkyl or C₃-C₈cycloalkyl; each of —C₁-C₆alkyl or C₃-C₈cycloalkyl is optionally substituted with at least one substituent selected from hydrogen, halogen, —C₁₋₆alkoxy;
R¹⁰ is each independently selected from hydrogen, halogen, —C₁-C₈alkyl, —C₂₋₈alkenyl, —C₂₋₈alknyl, C₃-C₈cycloalkyl, 3- to 8-membered heterocyclyl, C₆-C₁₂aryl, 5- to 12-membered heteroaryl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b} or —NR^{10a}SO₂R^{10b} or —CN; each of —C₁-C₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl is optionally substituted with at least one R^{10c};
R^{10a} and R^{10b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₂aryl, or 5- to 12-membered heteroaryl, each of said —C₁-C₈alkyl, —C₂-C₈alkenyl, —C₃-C₈alkynyl, C₃-C₈cycloalkyl, 3- to 8-membered heterocyclyl, C₆-C₁₂aryl, or 5- to 12-membered heteroaryl is optionally substituted with at least one substituent R^{10d};
R_{10c} and R^{10d} are each independently selected from halogen, hydrogen, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{10e}R^{10f}, —OR^{10e}, SR^{10e}, —SO₂R^{10e}, —SO₂NR^{10e}R^{10f}, —C(O)R^{10e}, —CO₂R^{10e}, —C(O)NR^{10e}R^{10f}, —NR^{10e}COR^{10f}, —NR^{10e}CO₂R^{8f} or —NR^{10e}SO₂R^{10f} or —CN;
R^{10e} and R^{10f} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R^{11a}, R^{11b}, R^{11c}, R^{11d}, R^{12a}, R^{12b}, R^{12c} and R^{12d} are each independently absence, oxo, hydrogen, halogen, —C₁₋₈alkyl, —C₂₋₈alkenyl, —C₂₋₈alkynyl, —C₁₋₈alkoxy or —C₃₋₈cycloalkyl; each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy or —CN;
L¹ is independently selected from —O—, —NR^{a}—, —C(O)—, **^{L1}-C(O)NR^{a}—**^{L1}, *^{L1}-C(O)O—**^{L1}, *^{L1}-NR^{a}C(O)—**^{L1}, *^{L1}-OC(O)—**^{L1}, 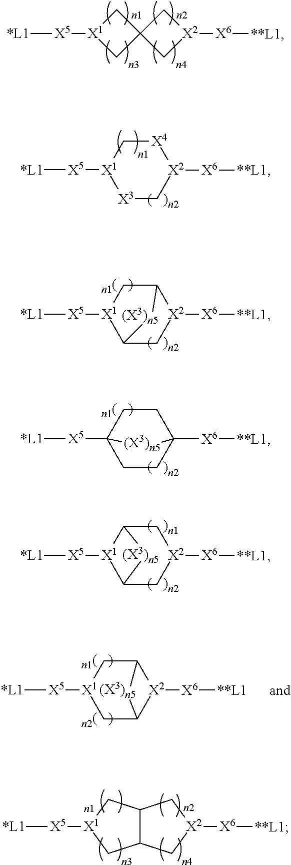
wherein each of said 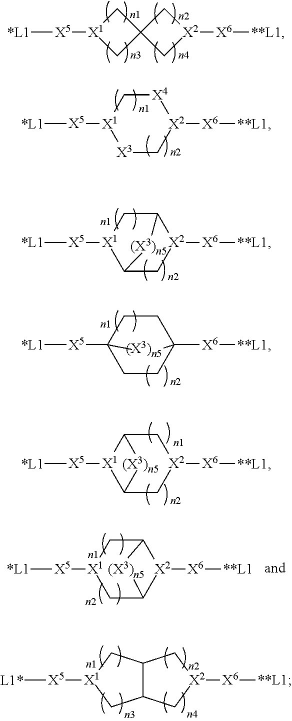
is optionally substituted with at least one R^{L1c};
wherein *^{L1} refers to the position attached to the 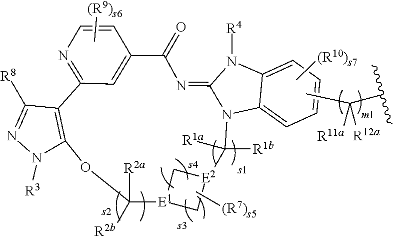
moiety, and **^{L1} refers to the position attached to the 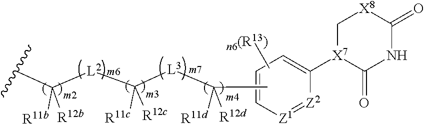
moiety;
L² is independently selected from —O—, —NR^{a}—, —C(O)—, *^{L2}-C(O)NR^{a}—**^{L2}, *^{L2}-C(O)O—**^{L2}, *^{L2}-NR^{a}C(O)—**^{L2}, -*^{L2}-OC(O)—**^{L2}, 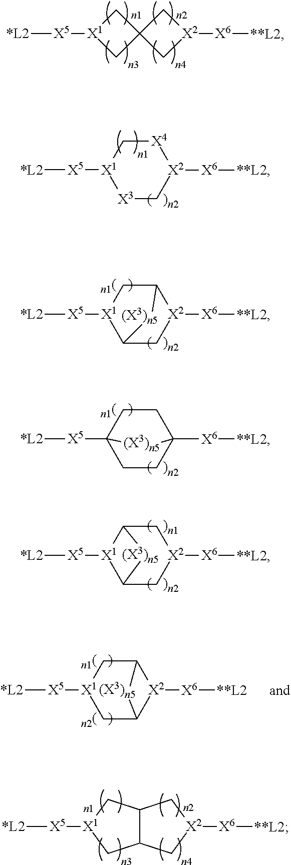
wherein each of said 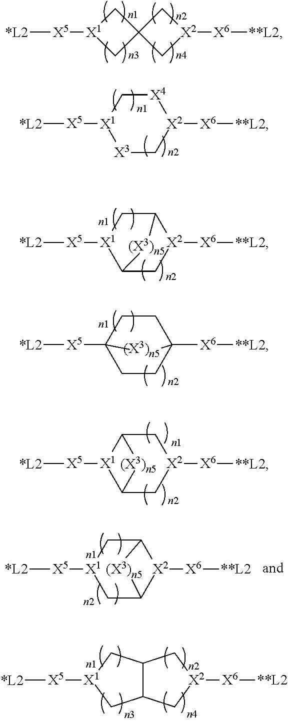
is optionally substituted with at least one R^{L2c};
wherein *^{L2} refers to the position attached to the 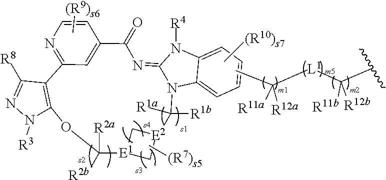
moiety, and **^{L2} refers to the position attached to the 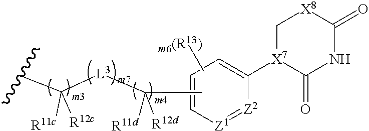
moiety;
L³ is independently selected from —O—, —NR^{a}—, —C(O), *^{L3}-C(O)NR^{a}—**^{L3}, *^{L3}-C(o)O—**^{L3}, *^{L3}-NR^{a}C(O)—**^{L3}, *^{L3}-OC(O)—**^{L3}, 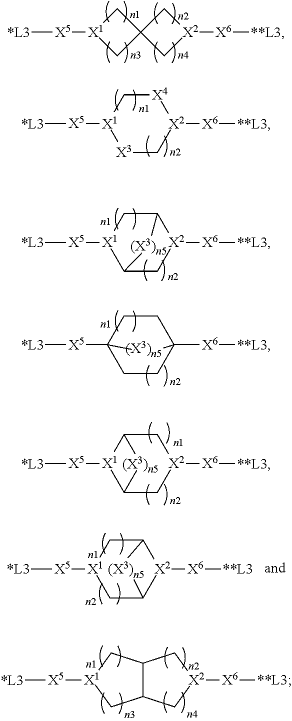
wherein each of said 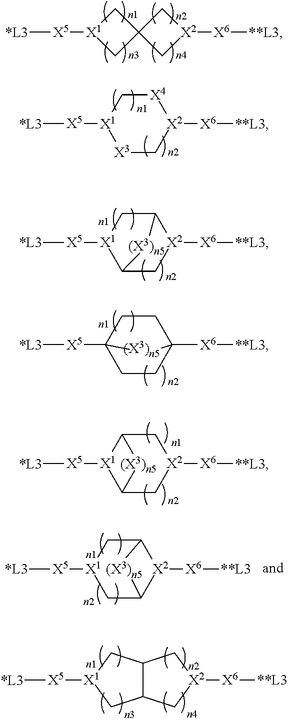
is optionally substituted with at least one R^{L3c};
wherein *^{L3} refers to the position attached to the 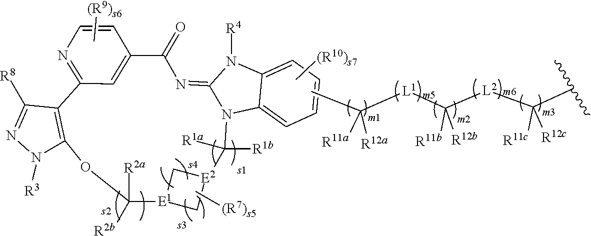
moiety, and **^{L3} refers to the position attached to the 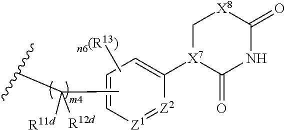
moiety;
each of said R^{L1c}, R^{L1c} and R^{L3c} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, from a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, from a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, from a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
Z¹ and Z² are each independently N or CR^{z};
R^{z}, at each occurrence, is independently selected from absence, hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
R^{Za} and R^{Zb} are each independently selected from absence, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ is each independently selected from absence, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of —C₁₋₈alkyl, —C₂₋₈alkenyl, —C₂₋₈alkynyl —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₂₂aryl, 5- to 12-membered heteroaryl, oxo, —CN, —OR^{13e}, —SO₂R^{13c}, —SO₂NR^{13e}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absence, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
at each occurrence, X¹, X² and X⁷ are each independently selected from —CR^{a}, or N;
at each occurrence X³, X⁴ and X⁸ are each independently selected from —NR^{a}—, —O—, —S— and —CR^{a}R^{b}—;
at each occurrence, X⁵ and X⁶ are each independently selected from absence, single bond, —C(O)—, —NR^{a}— and —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alknyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₂aryl or 5- to 12-membered heteroaryl;
m1, m2, m3 and m4 are each independently 0, 1 or 2; provided that m1+m2+m3+m4≤4;
m5, m6 and m7 are each independently 0, 1 or 2; provided that m5+m6+m7≥1;
n1, n2, n3, n4 and n5 are each independently 0, 1, 2 or 3;
n6 is each independently 0, 1, 2, 3 or 4;
s1 and s2 are each independently 0, 1, 2 or 3;
s3 and s4 are each independently 1, 2 or 3;
s5, s6 and s7 are each independently 0, 1, 2 or 3;
provided that:
for any one of L¹, L² or L³, when X¹ is N, X⁵ is single bond, absence, —C(O)—; and/or when X² is N, X⁶ is single bond, absence, —C(O)—;
when L¹ is 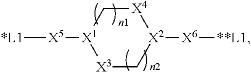
when X¹ is N, X⁵ is single bond, absence, —C(O)—; and/or X³ is —CR^{a}R^{b}—; when X² is N X⁶ is single bond, absence, —C(O)—, and/or X⁴ is CR^{a}R^{b};
when L² is 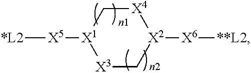
when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L³ is 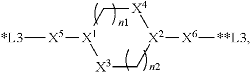
when X¹ is N, X⁵ is single bond, absence, —C(O)—; and/or X³ is —CR^{a}R^{b}—; when X² is N X⁶ is single bond absence, —C(O)—, and/or X⁴ is —CR^{a}R^{b}—.

2. The compound of claim 1, wherein the compound is selected from formula (Iia), (Iib), (IIIa), (IIIb), (Iva), (Ivb), (Va), (Vb), (Via), (Vib), (Vic), (Vid), (Vie), (Vif), (Vig), (Vih), (Vii), (Vij), (Vik), (Vil), (Vim), (Vin), (Vio), (Vip) and Vig):

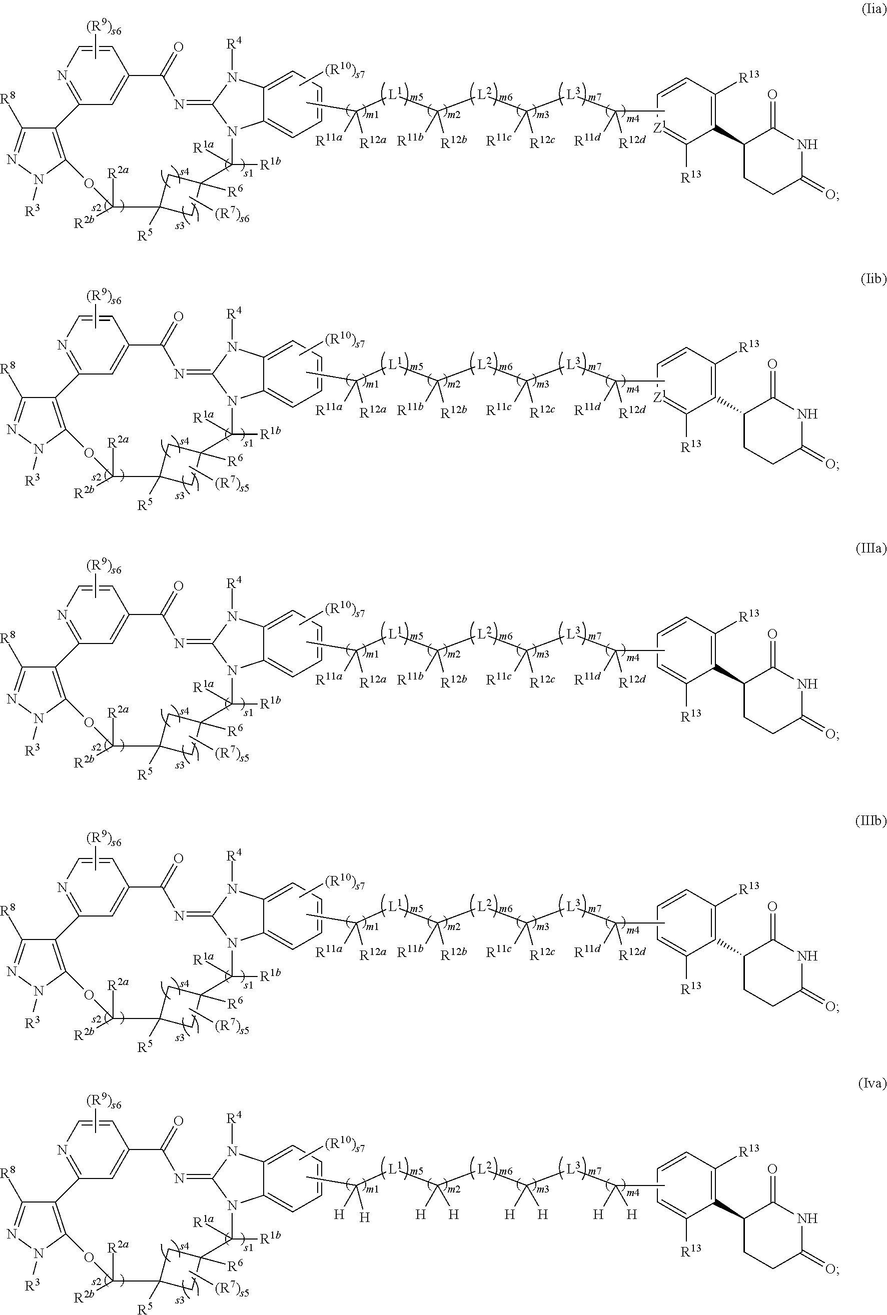

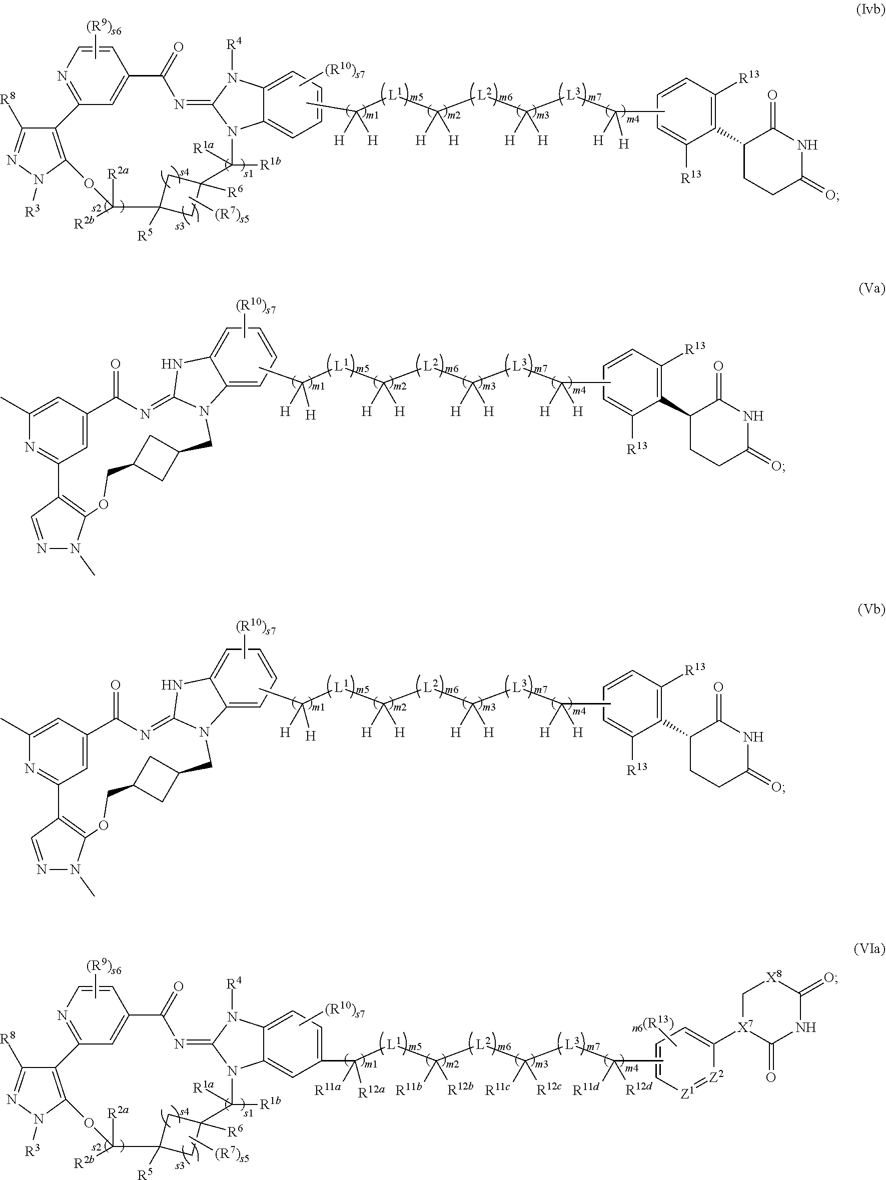

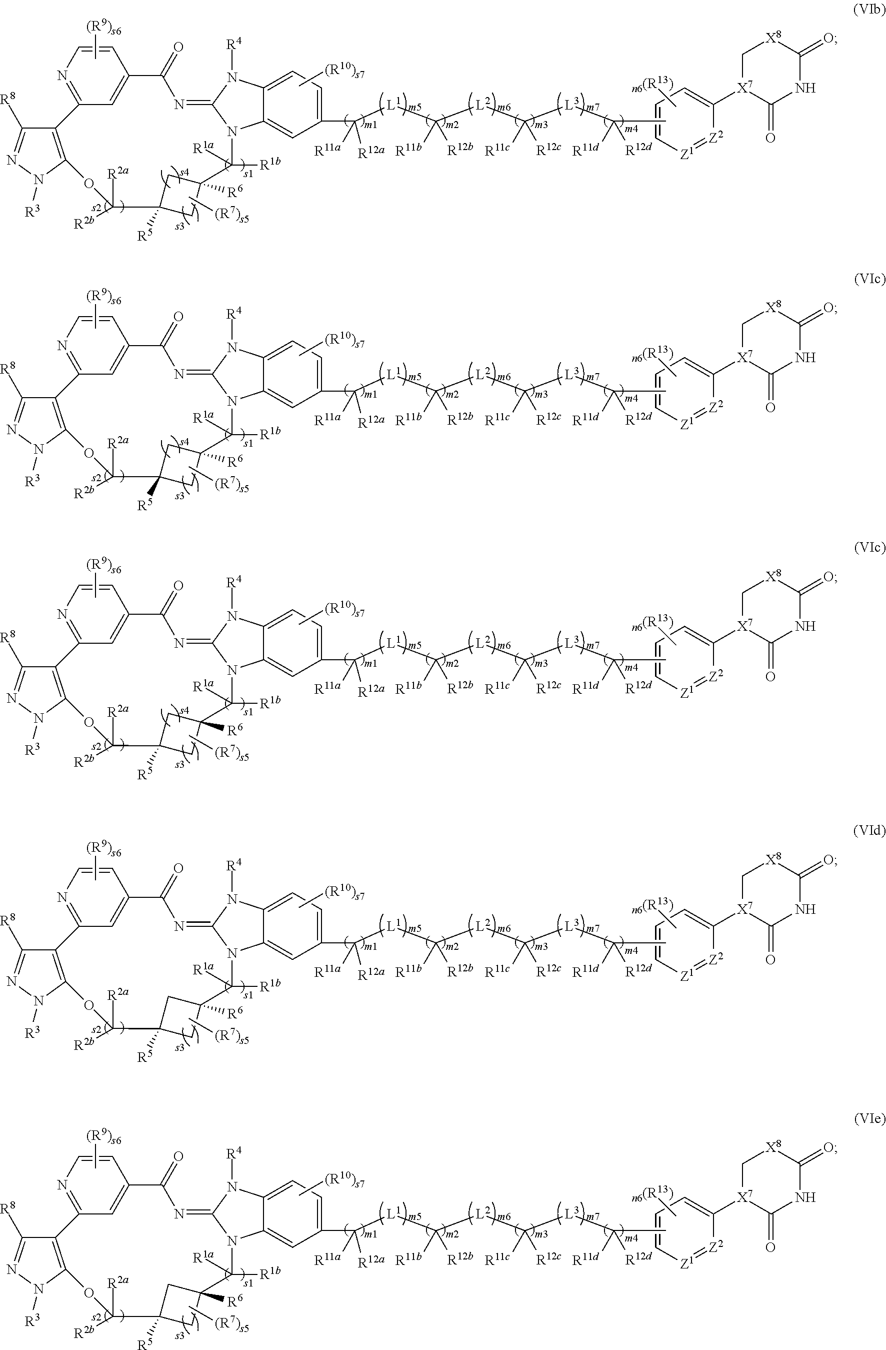

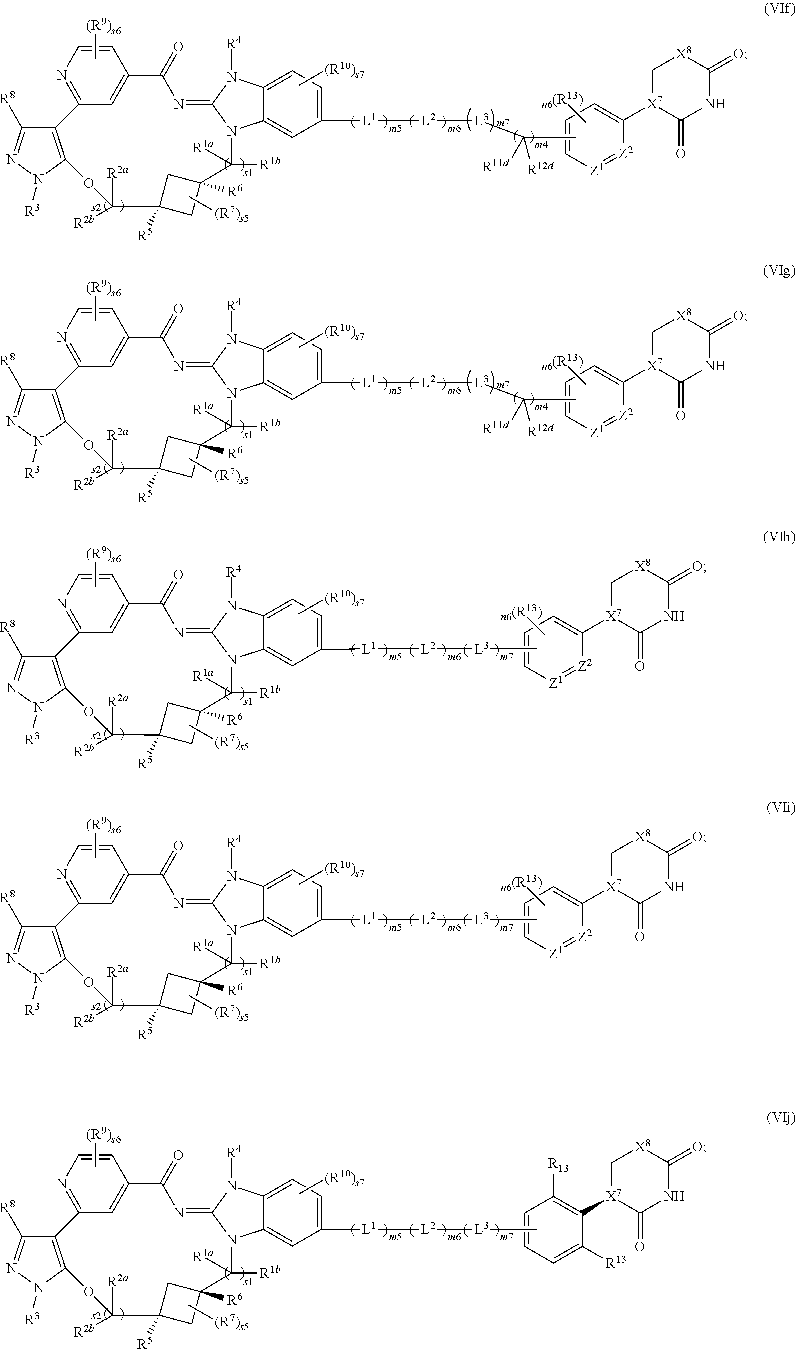

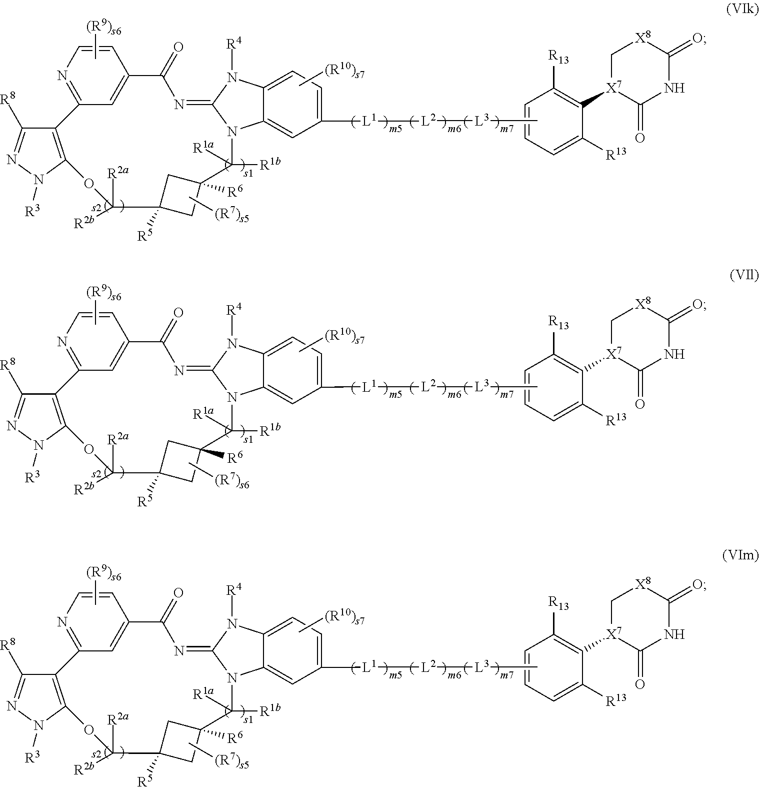

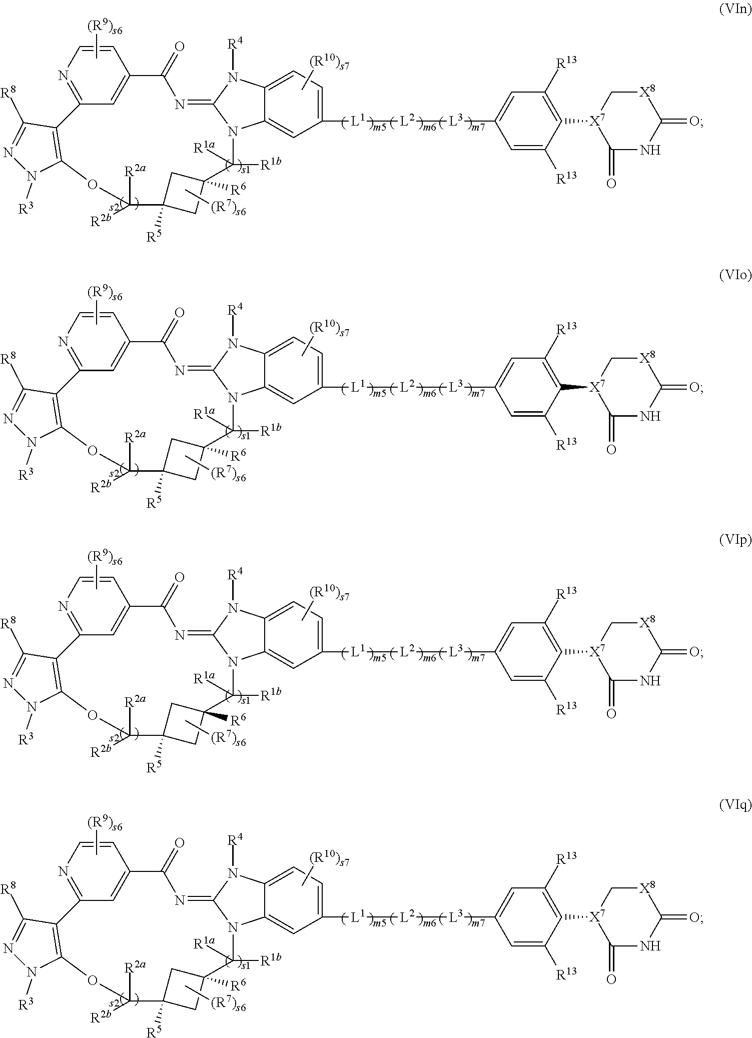

3. (canceled)

4. (canceled)

5. The compound of claim 1, wherein m1+m2+m3+m4=0, 1, 2 or 3; the number of —CH₂— in 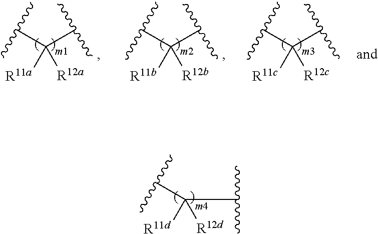
moieties is no more than 2;
R^{11a}, R^{11b}, R^{11c}, R_{11d}, R^{12a}, R^{12b}, R^{12c} and R^{12d} are each independently oxo, hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl.

6. (canceled)

7. The compound of claim 1,
R³ and R⁴ are each independently hydrogen, methyl, ethyl, propyl, butyl, pentyl, hexyl, heptyl, cyclobutyl, cyclopentyl, cyclohexyl, cycloheptyl or cyclooctyl; preferably R³ is independently methyl, and R⁴ is hydrogen.

8. The compound of claim 1, wherein R^{1a}, R^{1b}, R^{2a} and R^{2b}, are each independently hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN; wherein each said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl and cyclooctyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN.

9. The compound of claim 1, wherein R^{1a}, R^{1b}, R^{2a} and R^{2b} are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, —Cl₂CH₂OCH₂CH₃.

10. The compound of claim 1, wherein R⁵ and R⁶ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, —CH₂CH₂OCH₂CH₂; or
R⁵ and R⁶ with the carbon atoms to which they are attached, form a 3-, 4-, 5-, 6-7- or 8-membered ring, said ring comprising 0-3 heteroatoms independently selected from nitrogen, oxygen or sulfur; said ring is optimally substituted with at least one substituent F, Cl, Br, I, hydroxy, methyl, ethyl, propyl, butyl, pentyl, hexyl, heptyl or octyl;
R⁷ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, CH₂OCH₃, —CH₂OCH₂CH₃, —CH₂CH₂OCH₃, —CH₂CH₂OCH₂CH₃; or
two R⁷ with the carbon atom(s) to which they are attached, form a 3-, 4-, 5-, 6-, 7- or 8-membered ring said ring comprising 0-3 heteroatoms independently selected from nitrogen, oxygen or sulfur; said ring is optionally substituted with at least one substituent F, Cl, Br, I, hydroxy, methyl, ethyl, propel, butyl, pentyl, hexyl, heptyl or octyl.

11.-13. (canceled)

14. The compound of claim 1, wherein R⁸ and R⁹ are each independently selected from hydrogen, F, Cl, Br, I, methyl, ethyl, propyl, butyl, pentyl, hexyl, cyclopropyl, cyclobutyl, cyclopentyl, cyclohexyl, cycloheptyl or cyclooctyl; said methyl, ethyl, propyl, butyl, pentyl, hexyl, cyclopropyl, cyclobutyl, cyclopentyl, cyclohexyl, cycloheptyl or cyclooctyl is optionally substituted with at least one substituent selected from hydrogen, F, Cl, Br, I, methoxy, ethoxy, propoxy, butoxy, pentoxy or hexoxy.

15. The compound of claim 1, wherein
R¹⁰ is each independently selected from hydrogen, —F, —Cl, —Br, —I, ethyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b} or —NR^{10a}SO₂R^{10b} or —CN each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl is optionally substituted with at least one R^{10c};
R^{10a} and R^{10b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl;
R^{10c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo(═O), —NR^{10e}R^{10f}, —OR^{10e} or —CN;
R^{10e} and R^{10f} t are each independently selected from hydrogen, methyl ethyl, propyl, buty, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

16. The compound of claim 1, wherein the 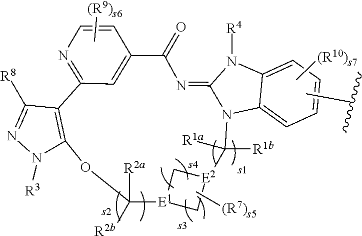
moiety is selected from

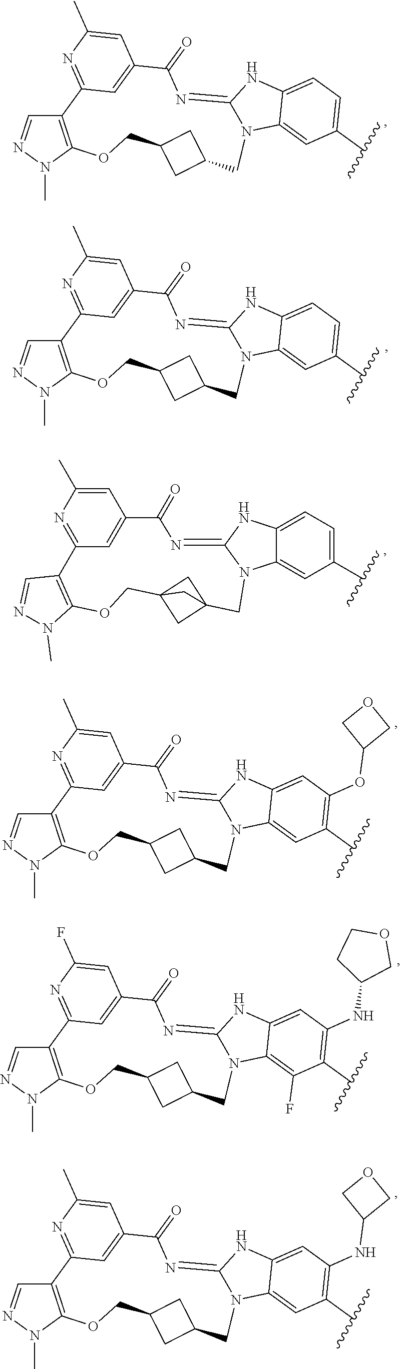

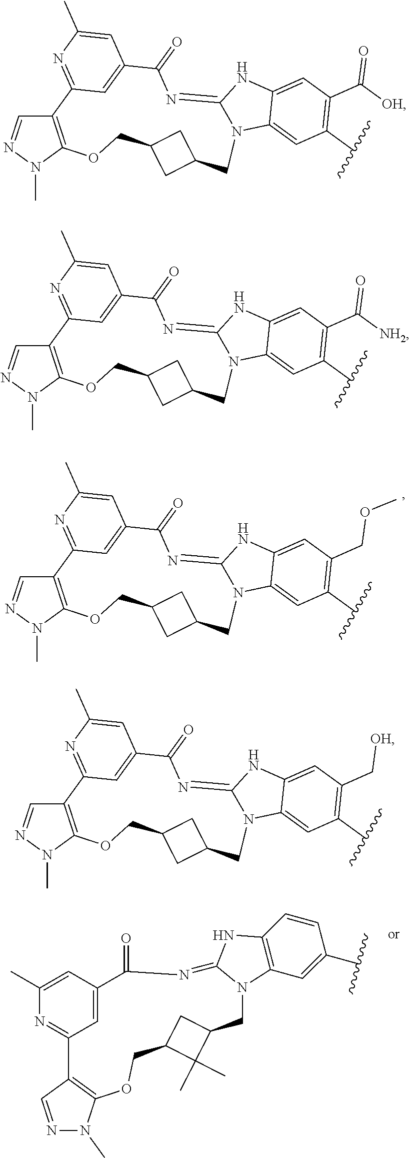

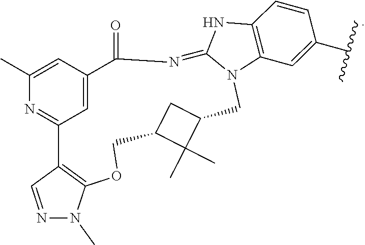 [SMILES: *c1ccc2c(c1)N1C[C@H]3C[C@@H](COc4c(cnn4C)-c4cc(cc(C)n4)C(=O)/N=C/1N2)C3(C)C]

17. (canceled)

18. (canceled)

19. The compound of claim 1, wherein L¹ is selected from —O—, —C(O)—, —N(R^{a})—, *^{L1}-C(O)N(R^{a})—**^{L1}, *^{L1}-C(O)O—**^{L1}, ^{*L1}—N(R^{a})C(O)—**^{L1}, *^{L1}-OC(O)—**^{L1}, 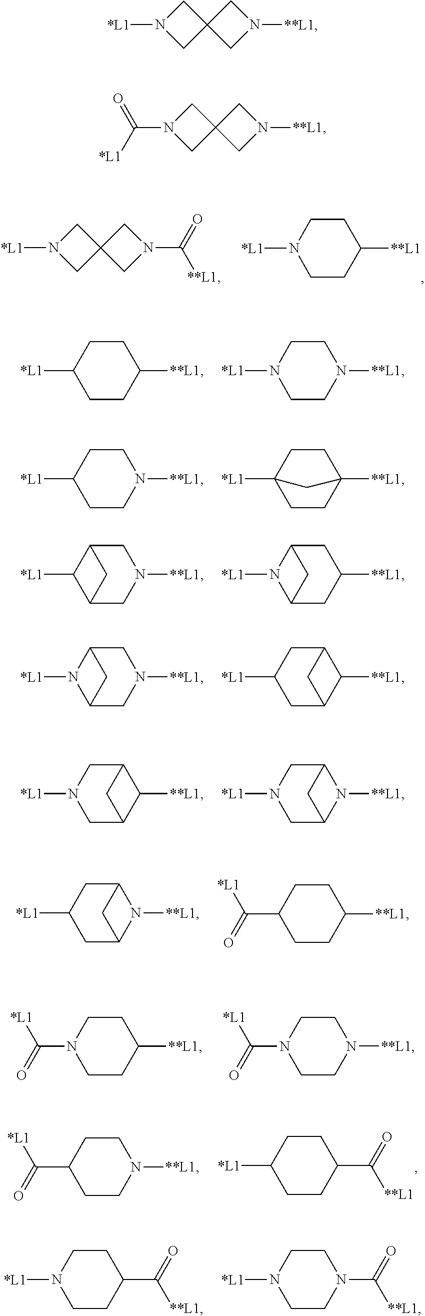 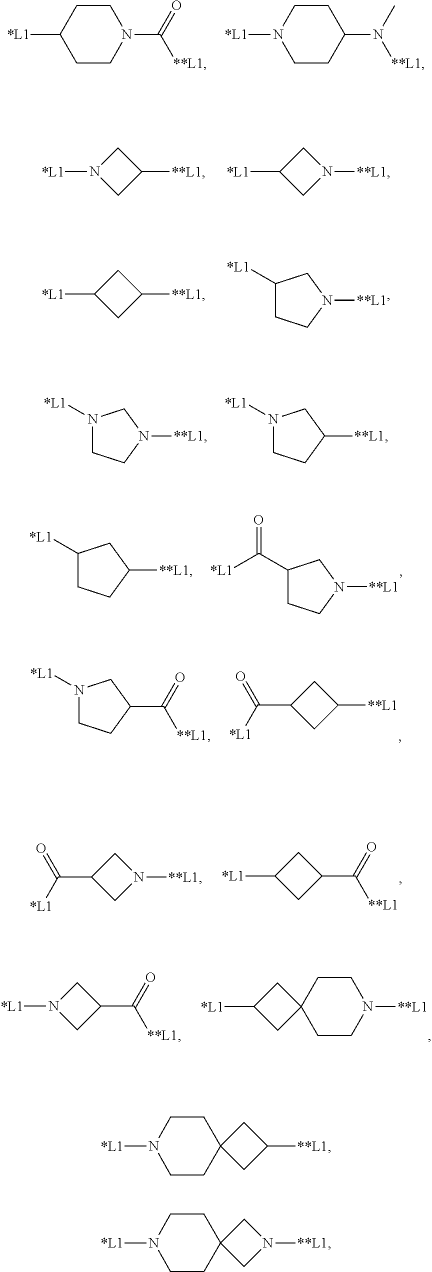 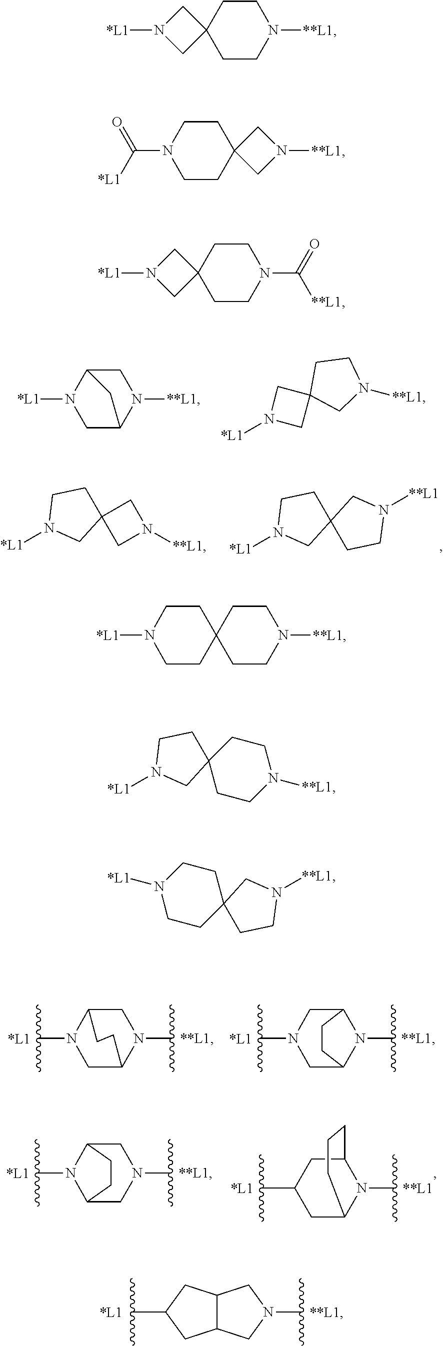 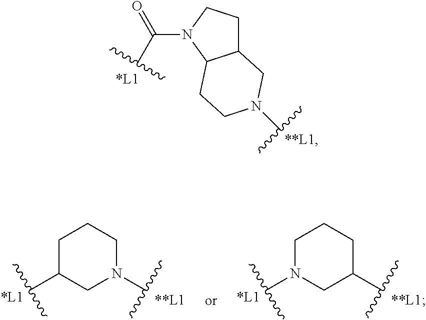
wherein each of said 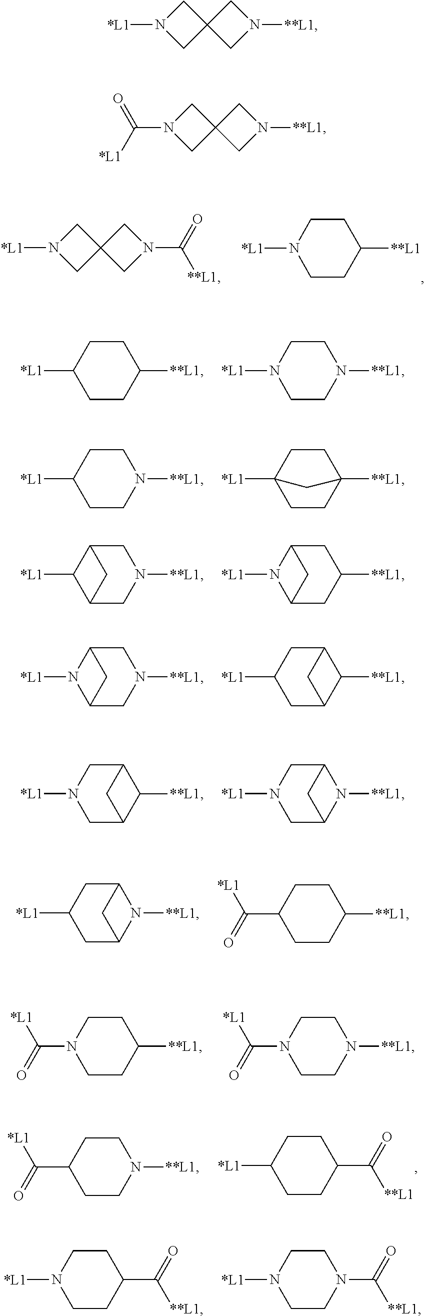 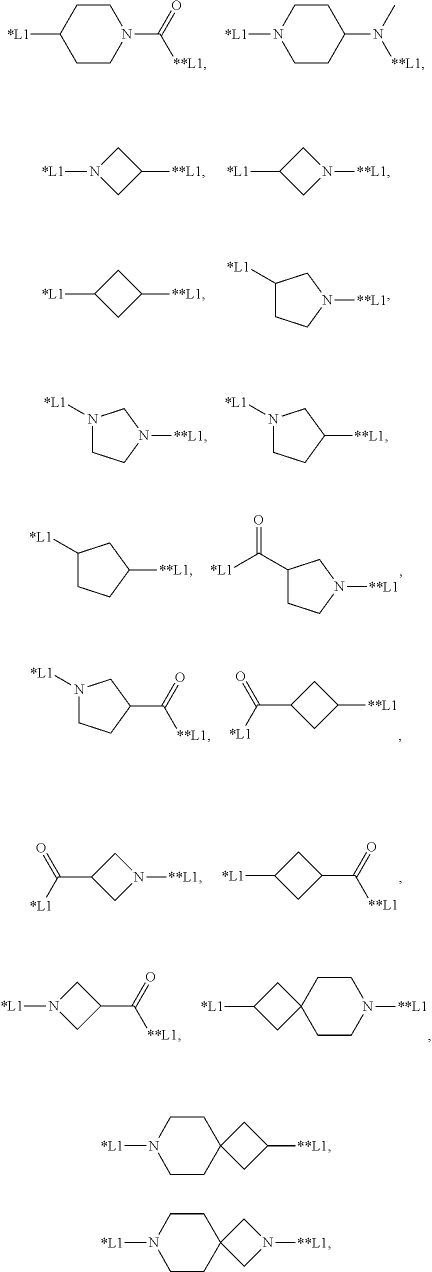 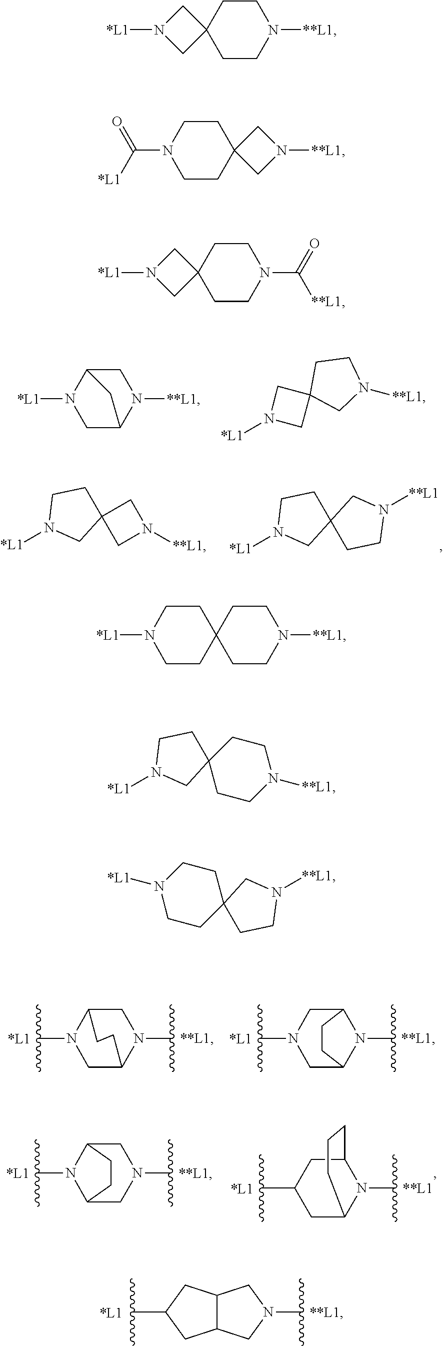 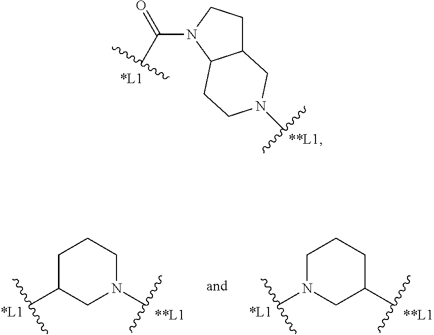
is optionally substituted with at least one R^{L1c};
each of said R is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, 4C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alknyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alknyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl 3- to 8-membered heterocyclyl, phenyl or 5- to 12-membered heteroaryl;
L² selected from —O—,_-C(O)—, —N(R^{a})—, *^{L2}-C(O)N(R^{a})—**^{L2}, *^{L2}-C(O)O—**^{L2}, *^{L2}-N(R^{a})C(O)—**^{L2}, *^{L2}-OC(O)—**^{L2}, 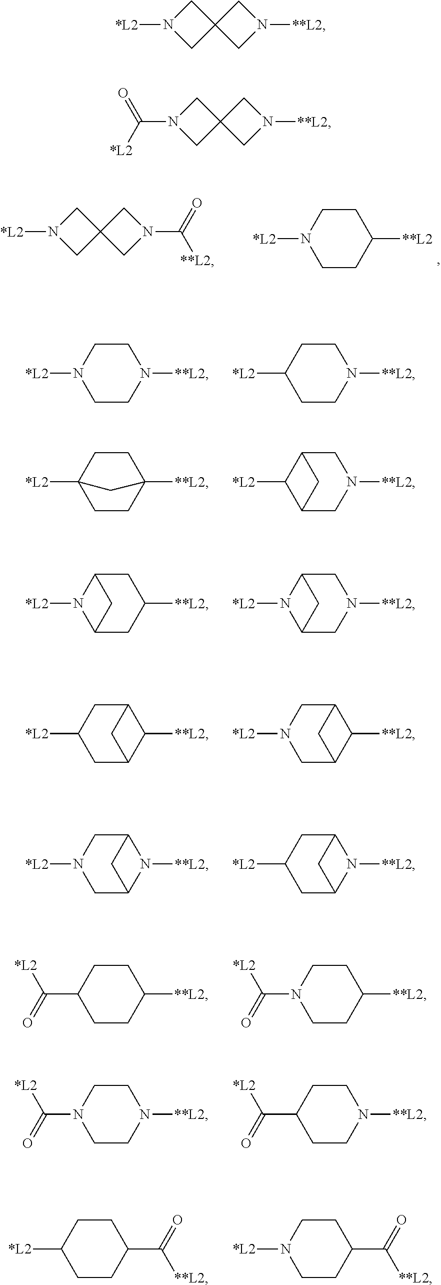 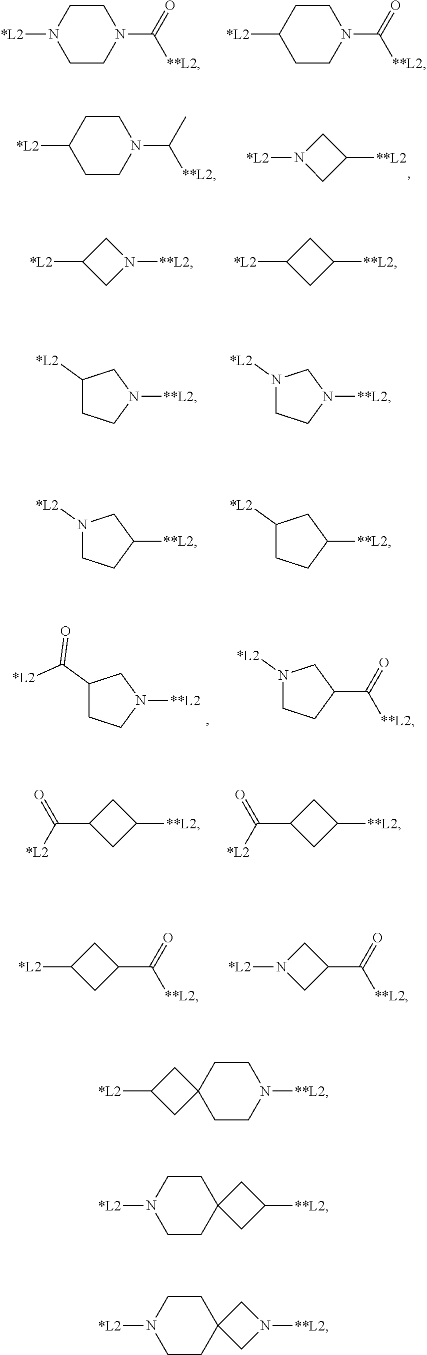 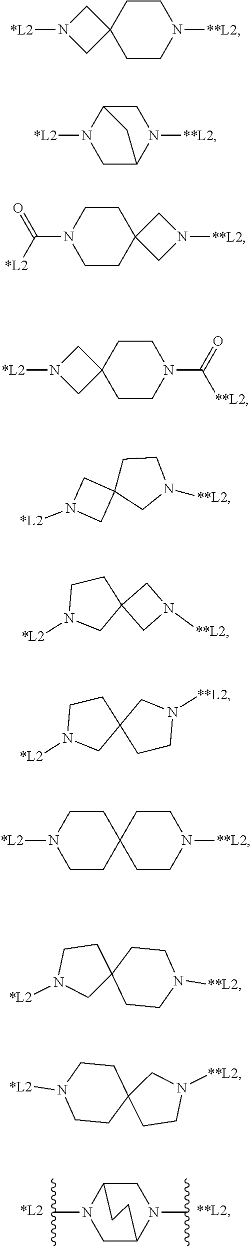 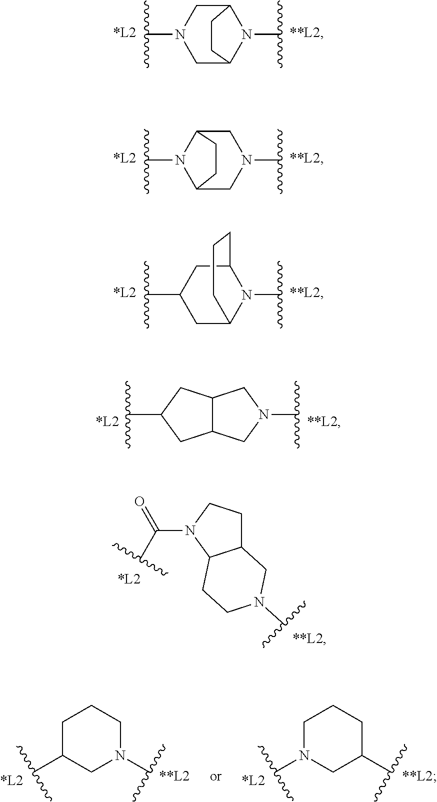
wherein each of said 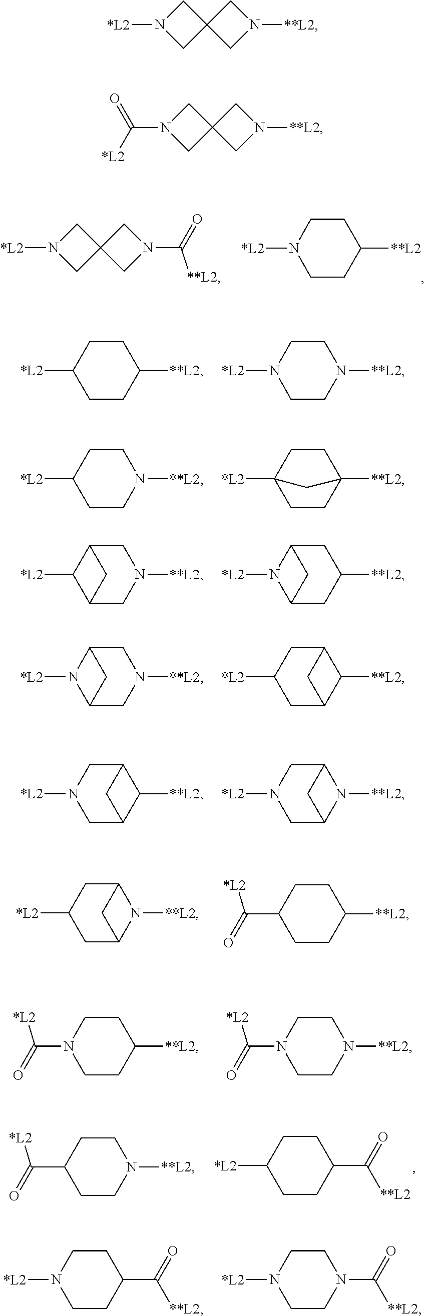 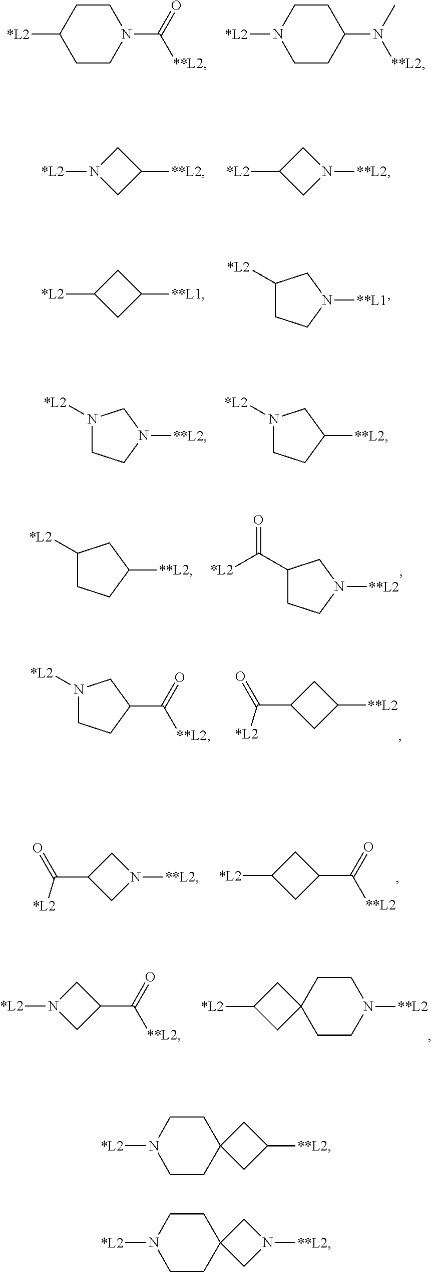 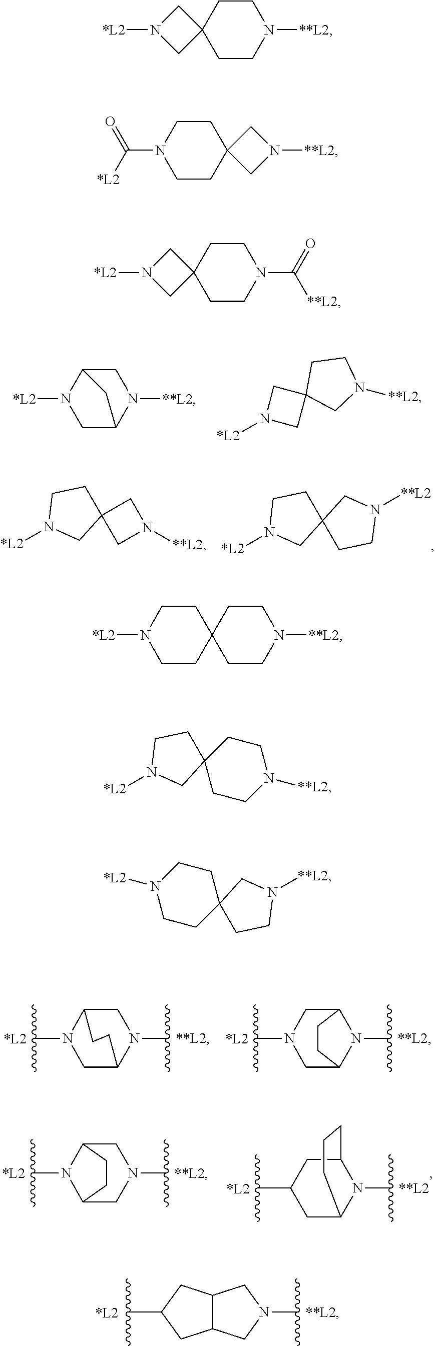 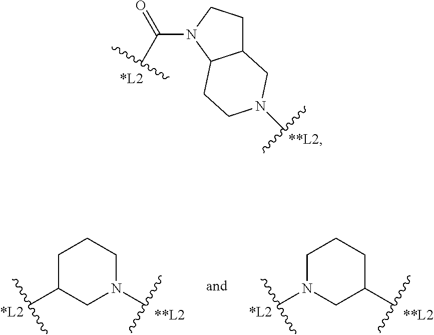
optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, 4C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alknyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alknyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl 3- to 8-membered heterocyclyl, phenyl or 5- to 12-membered heteroaryl;
L³ is selected from —O—, —N(R^{a})—, —C(O)—, *^{L3}-C(O)N(R^{a})—**^{L3}, *^{L3}-C(O)O—**^{L3}, *^{L3}-N(R^{a})C(O)—**^{L3}, *^{L3}-OC(O)—**^{L3} 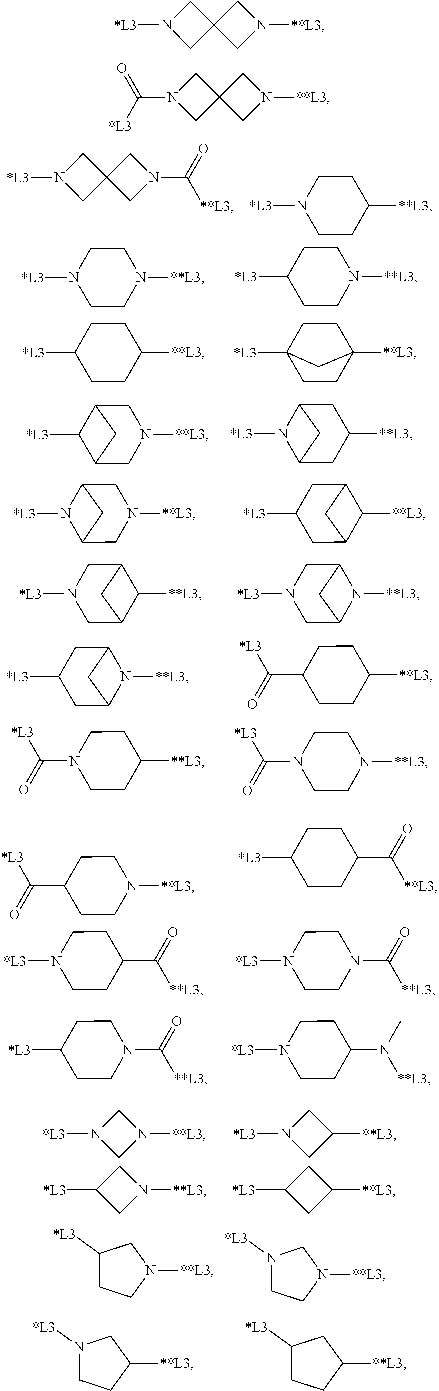 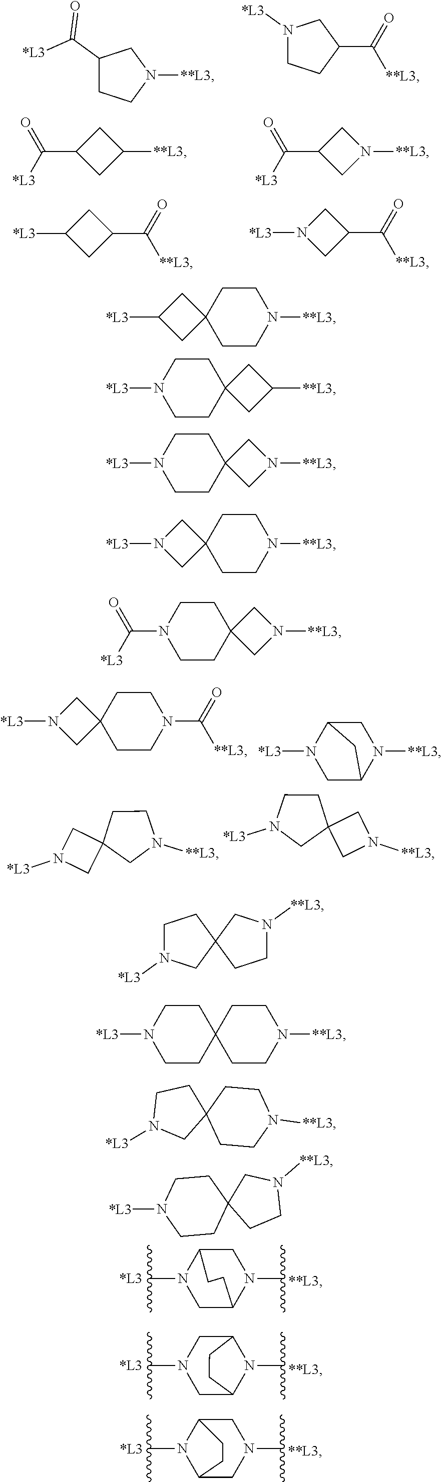 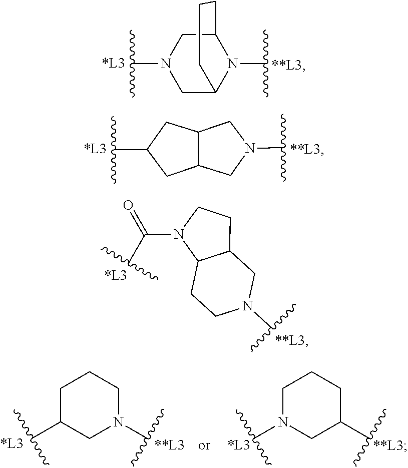
wherein each of said 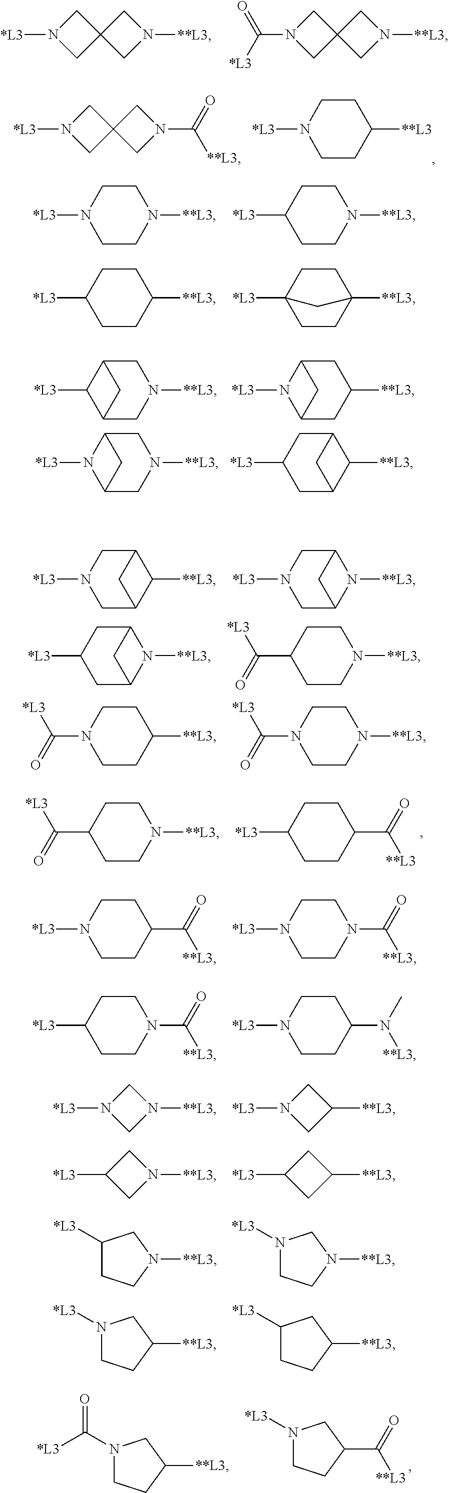 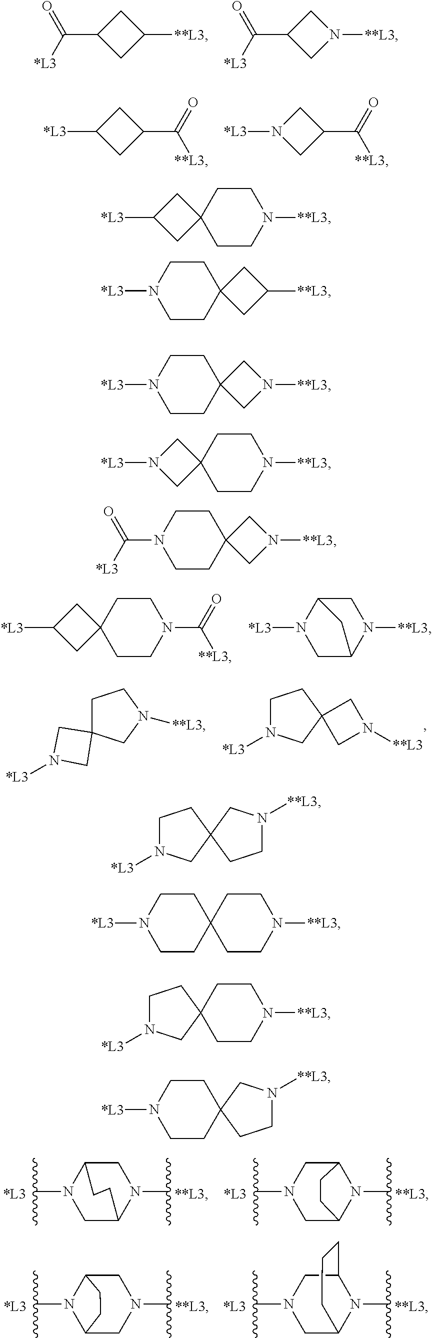 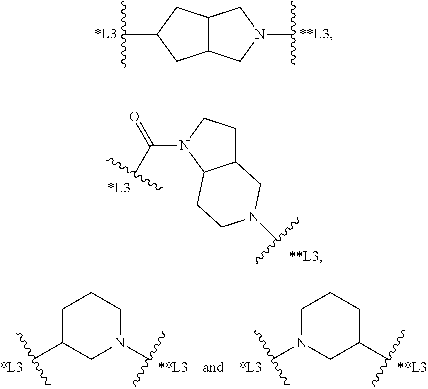 [SMILES: *C1CC2CN(*)CC2C1 ; *C(=O)N1CCC2CN(*)CCC21 ; *C1CCCN(*)C1 ; *C1CCCN(*)C1]
is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alknyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl 3- to 8-membered heterocyclyl, phenyl or 5- to 12-membered heteroaryl.

20. The compound of claim 1, wherein L¹ is selected from —O—, —N(CH₃)—, —C(O)—, —NH—, *^{L1}-C(O)N(CH₃)—**^{L1}, *^{L1}-(O)NH—**^{L1}, *^{L1}-C(O)O—**^{L1}, *^{L1}-C(O)N(C₂H₅)—**^{L1}, *^{L1}-C(O)N(C₃H₇)—**^{L1}, *^{L1}-N(CN₃)C(O)—**^{L1}, *^{L1}-NHC(O)—**^{L1}, *^{L1}-OC(O)—**^{L1}, *^{L1}-N(C₂H₅)C(O)—**^{L1}, *^{L1}-N(C₃H₇)C(O)—*^{L1}, 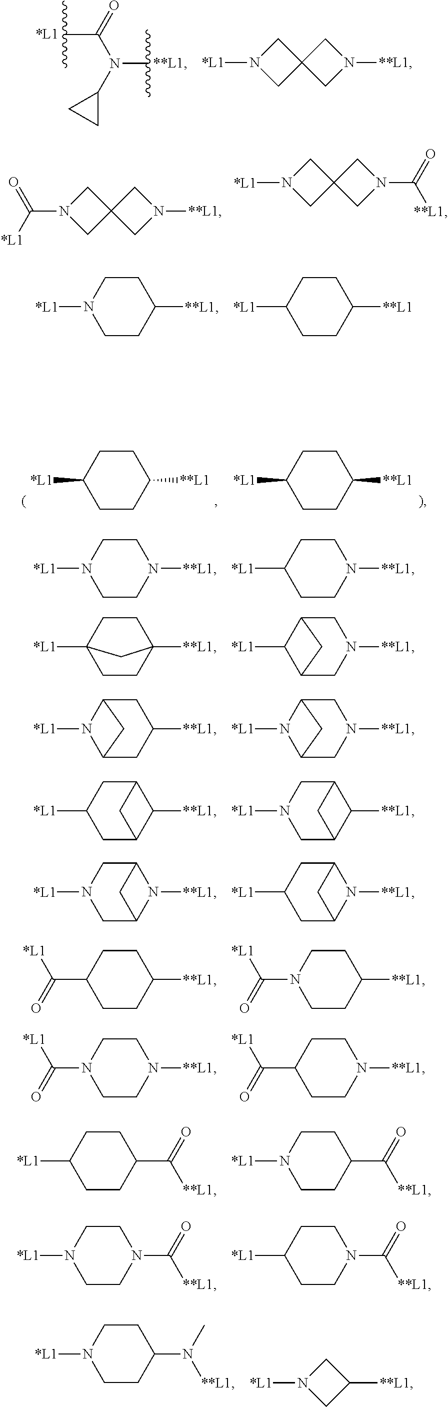 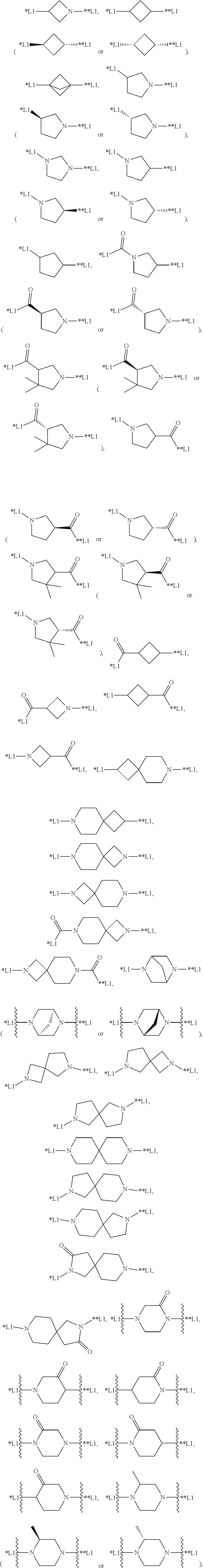 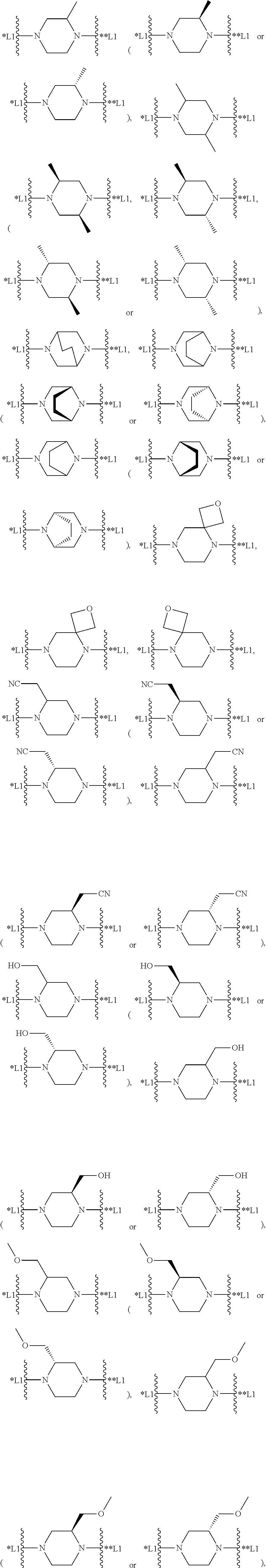 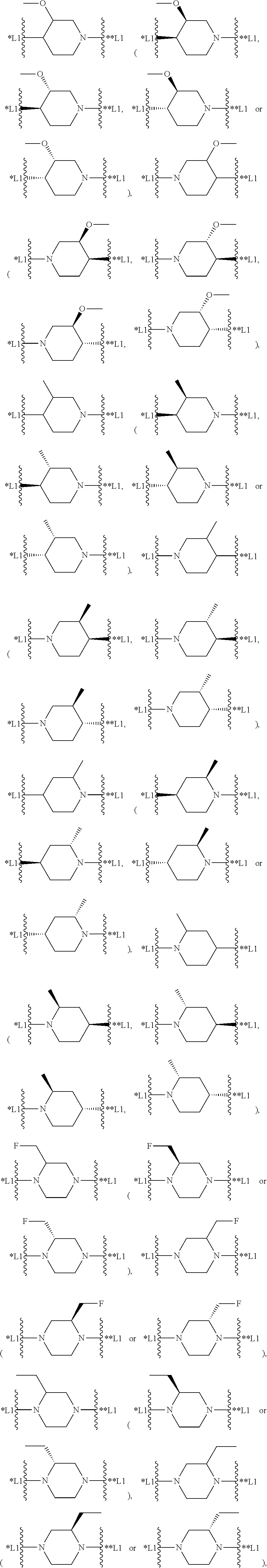 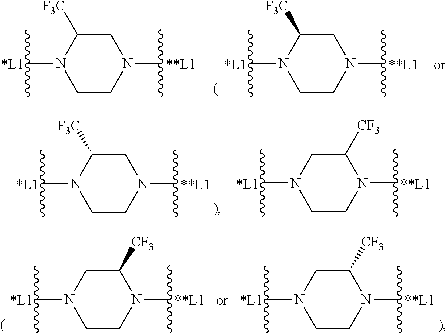 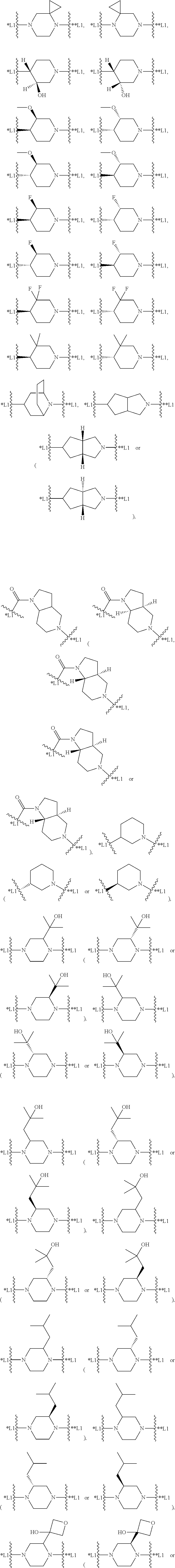 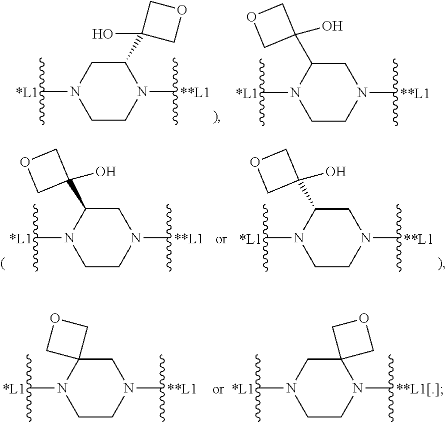
L² is selected from —O—, —N(CH₃)—, —C(O)—, —NH—, *^{L2}-C(O)N(CH₃)—**^{L2}, *^{L2}-C(O)NH—**^{L2}, *^{L2}-C(O)O—**^{L2}, *^{L2}-C(O)N(C₂H₅)—**^{L2}, *^{L2}-C(O)N(C₃H₇)—**^{L2}, *^{L2}-N(CH₃)C(O)—**^{L2}, *^{L2}-NHC(O)—**^{L2}, *^{L2}-OC(O)—**^{L2}, *^{L2}-N(C₂H₅)C(O)—**^{L2}, *^{L2}-N(C₃H₇)C(O)—**^{L2}, 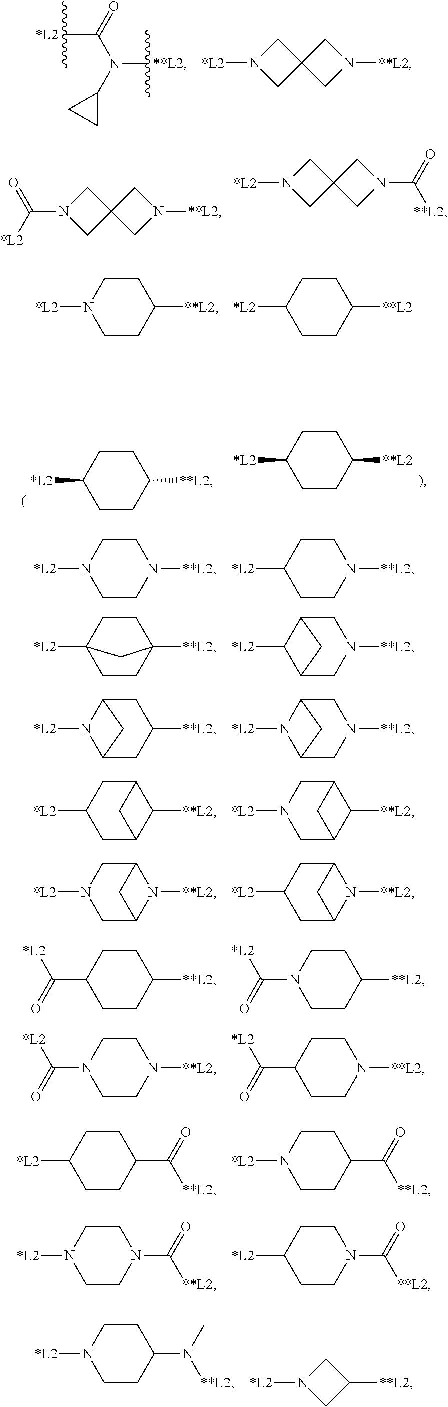 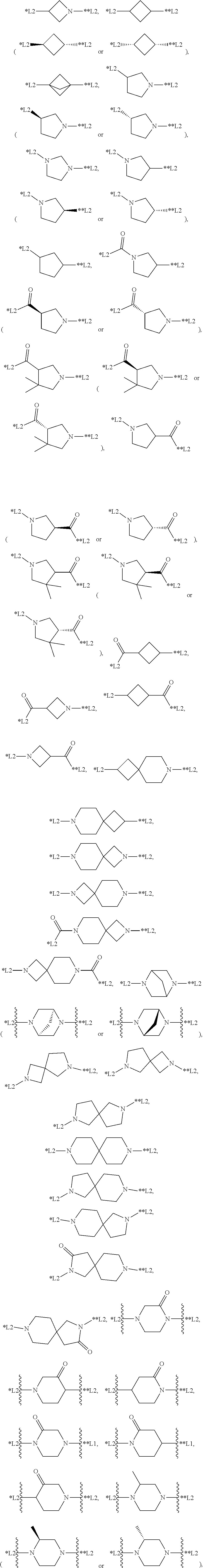 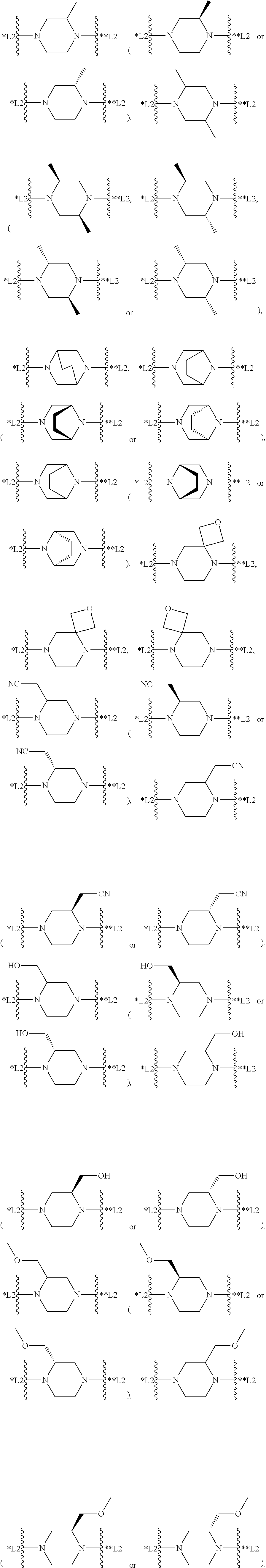 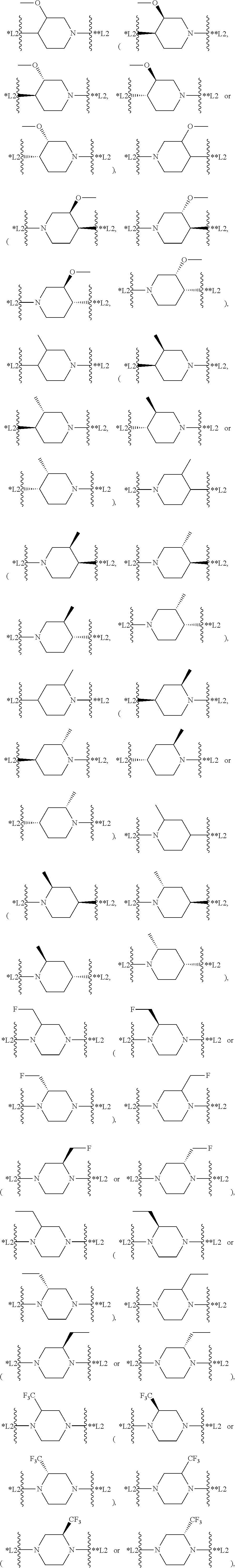 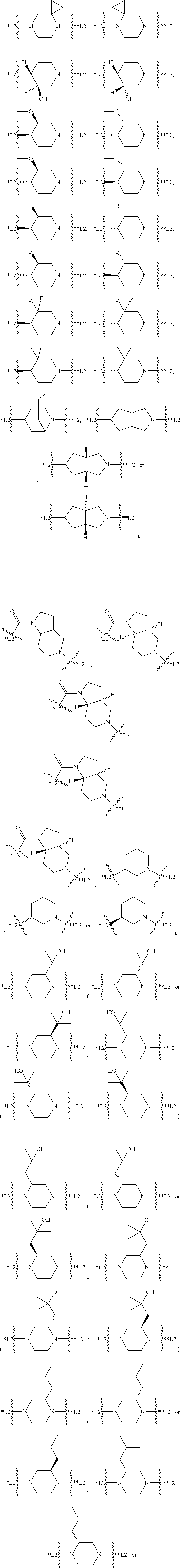 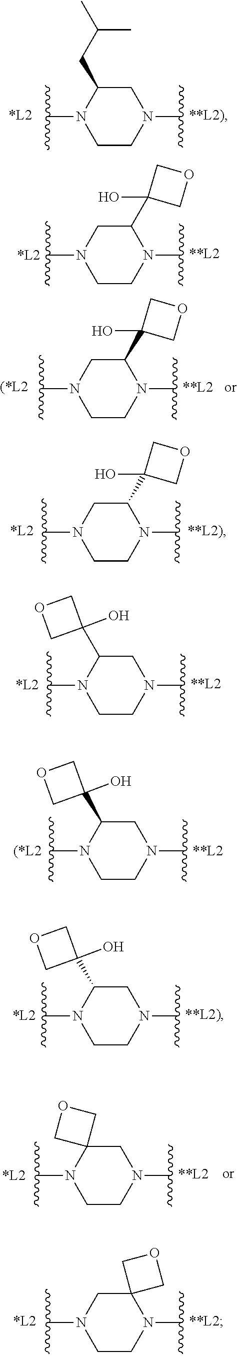
L³ is selected from —O—, —N(CH₃)—, —C(O)—, —NH—, *^{L3}-C(O)N(CH₃)—**^{L3}, *^{L3}-C(O)NH—**^{L3}, *^{L3}-C(O))—**^{L3}, *^{L3}-N(C₂H₅)C(O)—**^{L3}, *^{L3}-N(C₃H₇)C(O)—**^{L3},

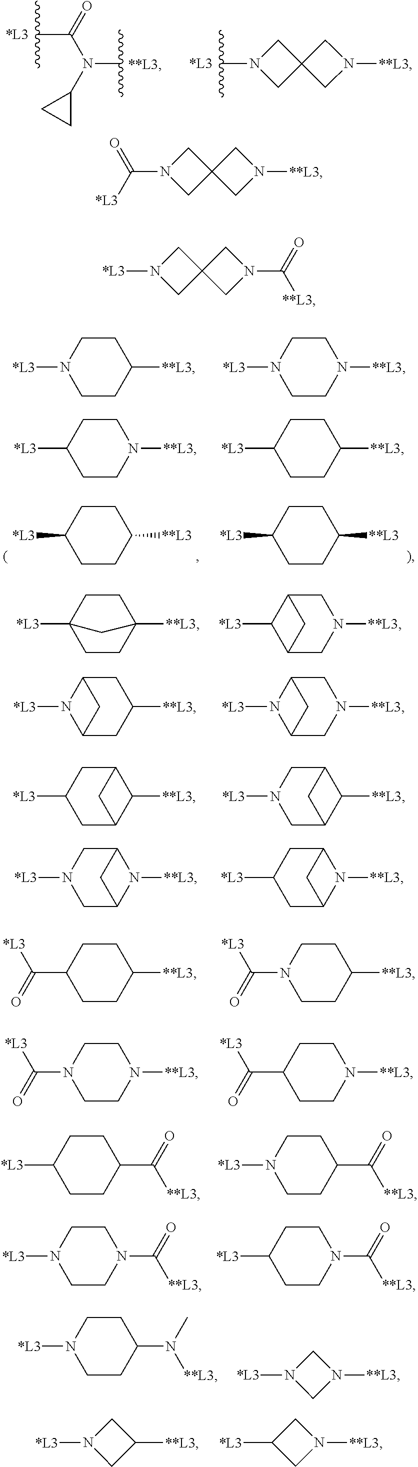

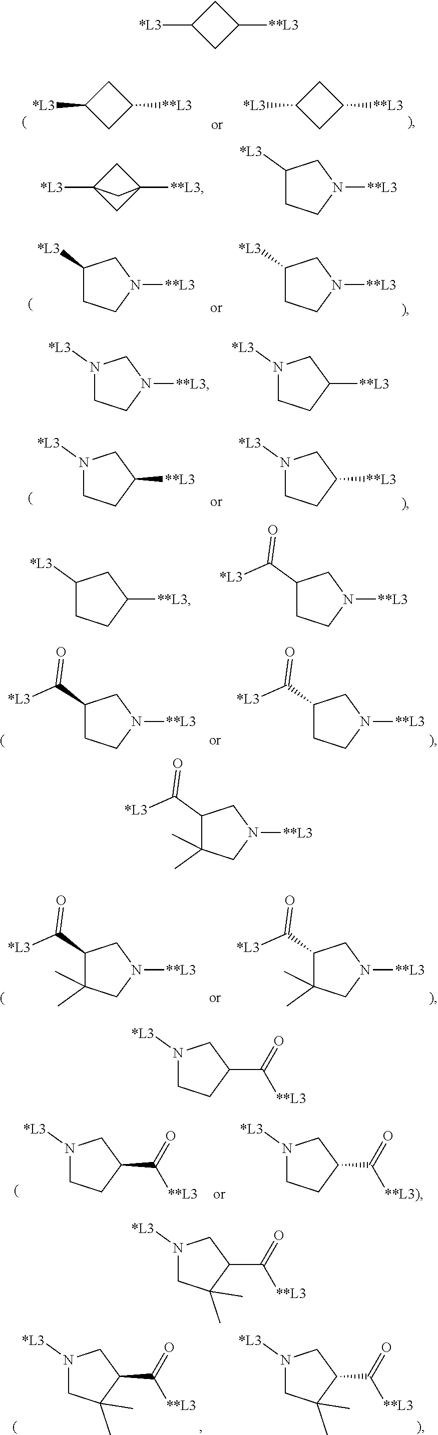

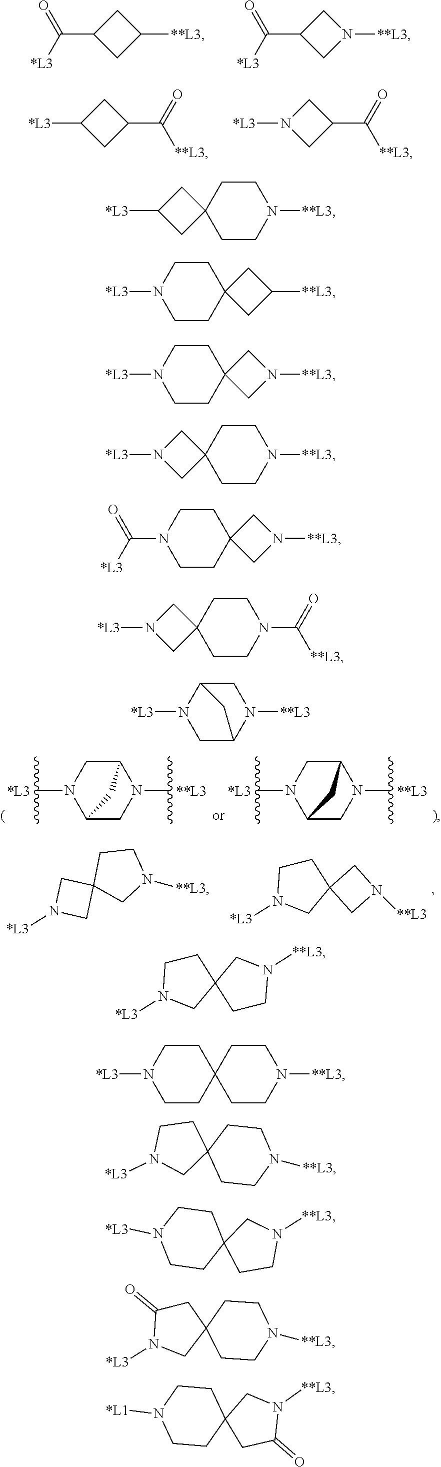

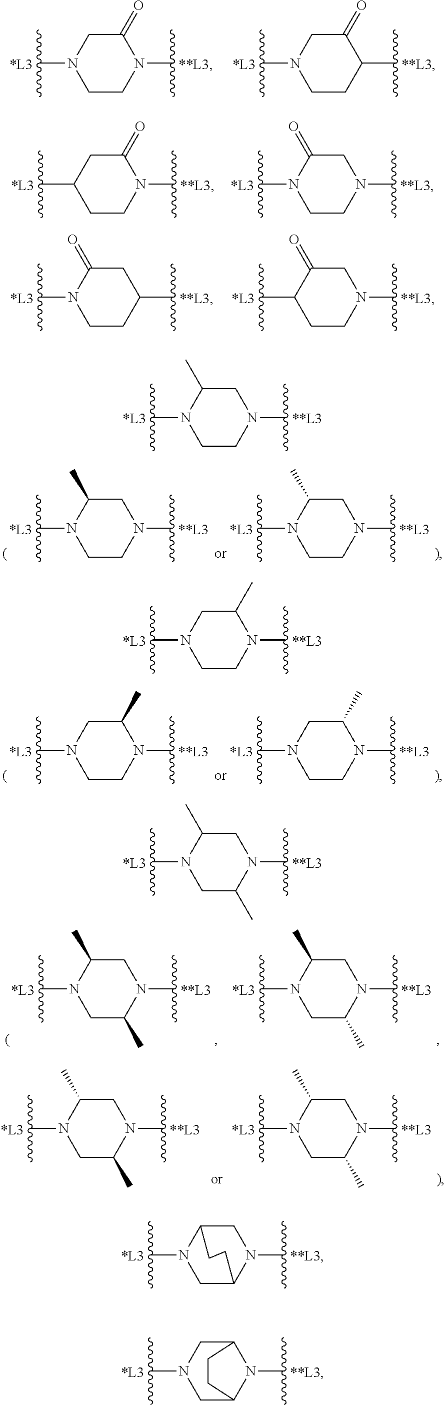

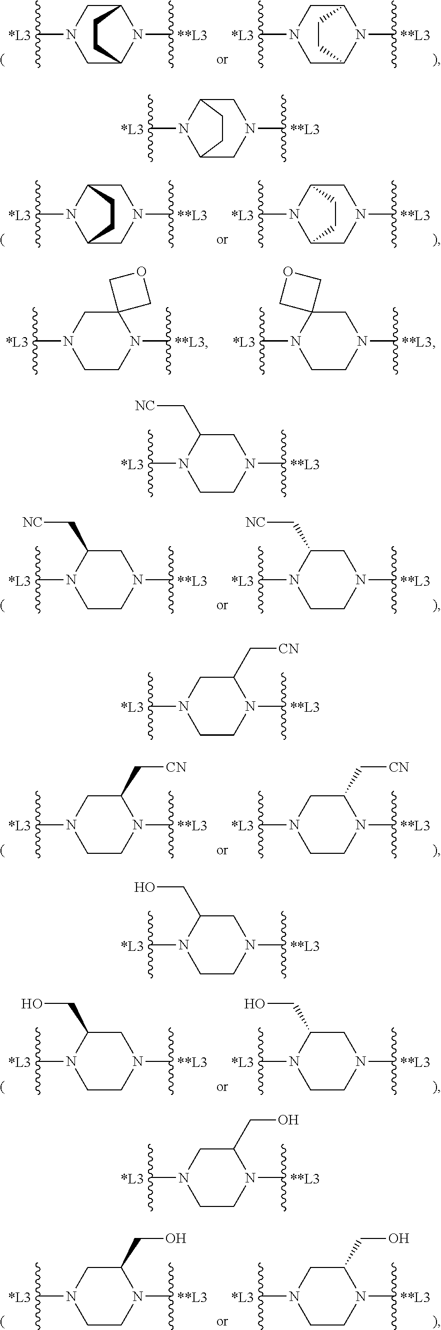

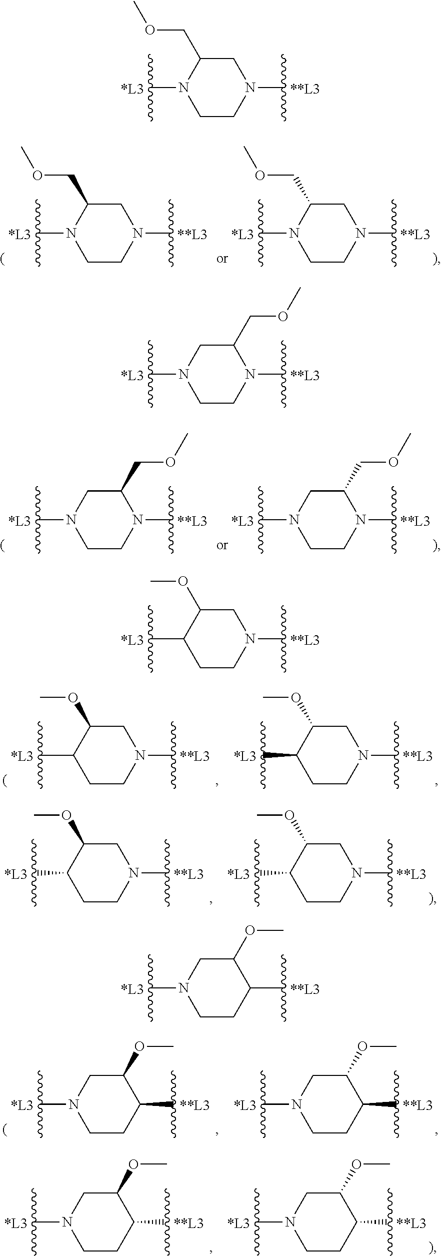

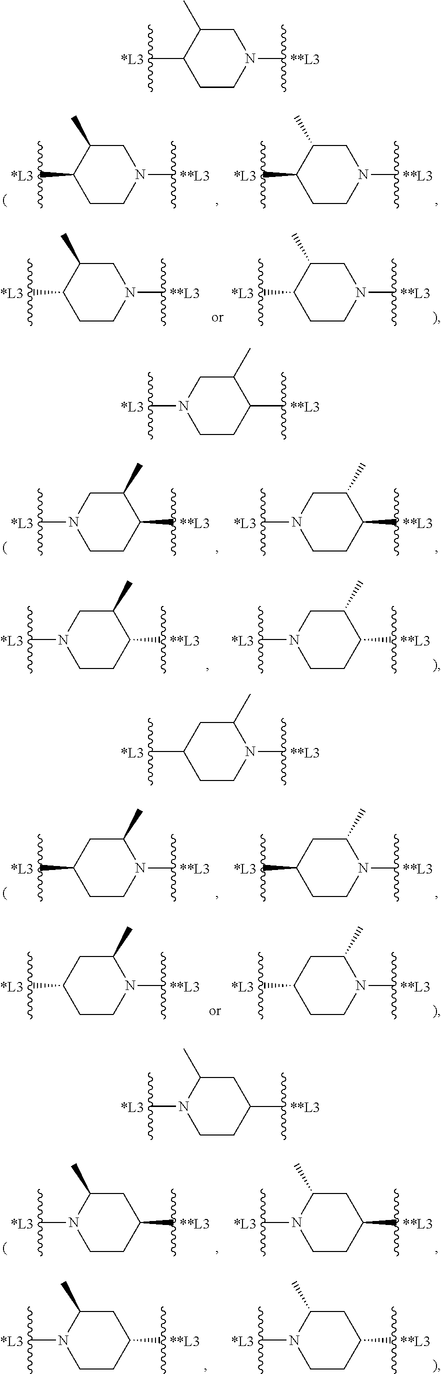

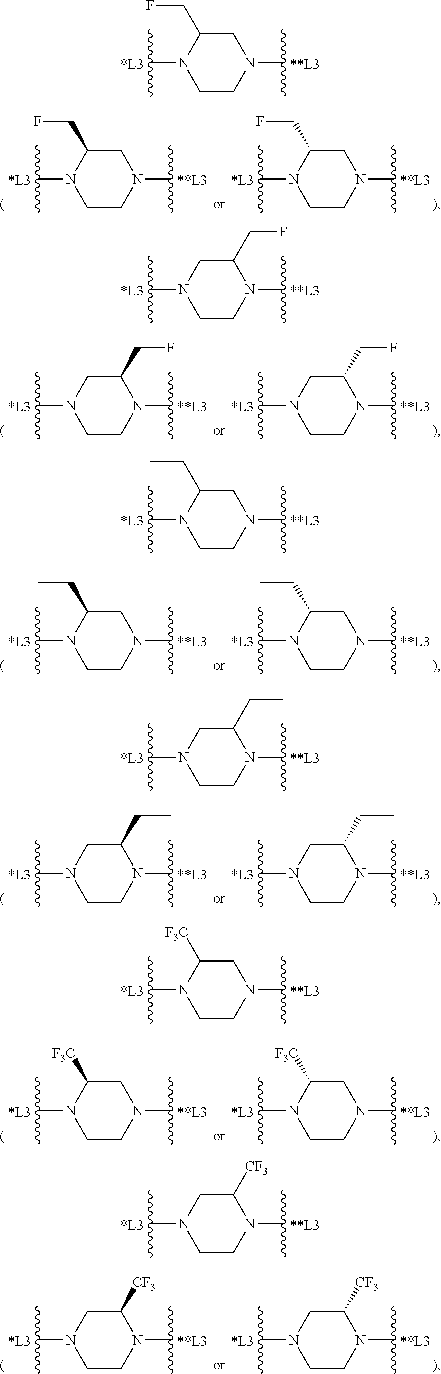

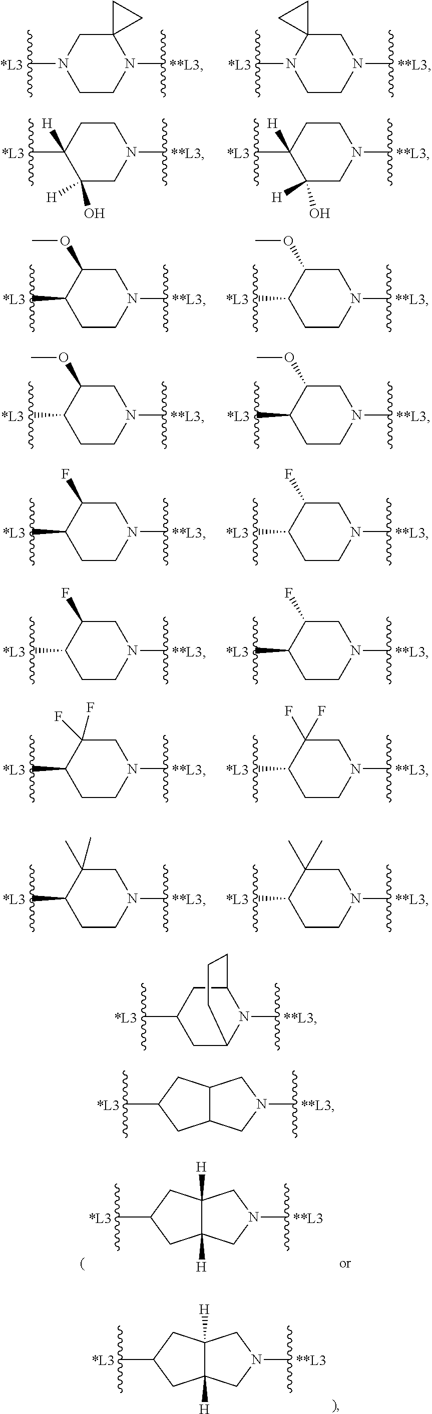

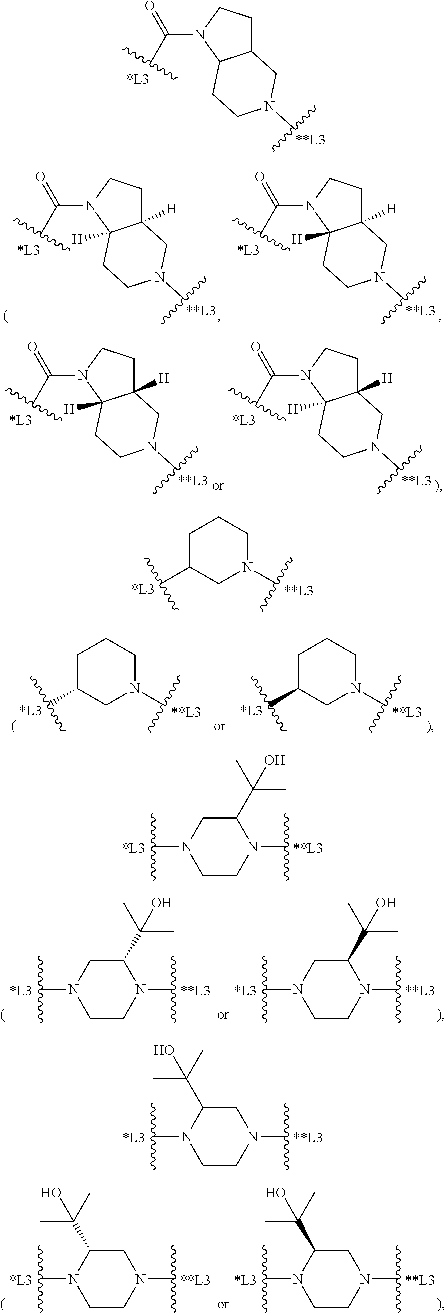

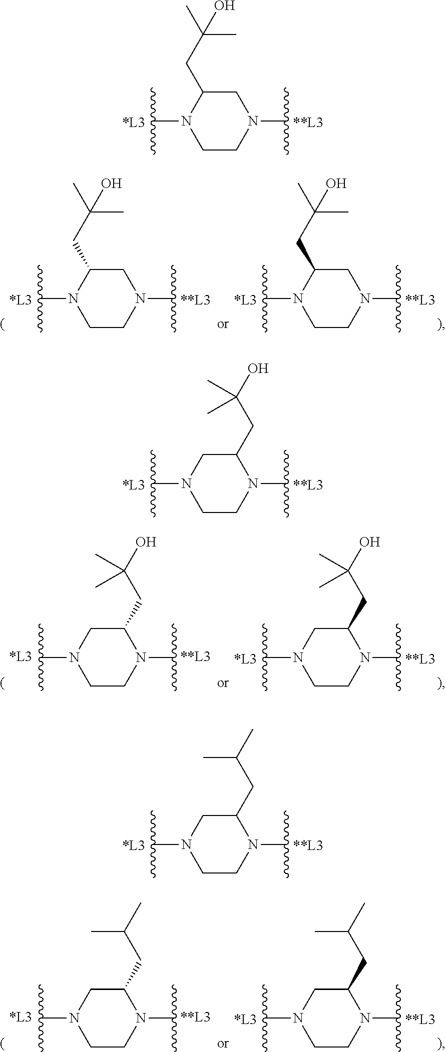

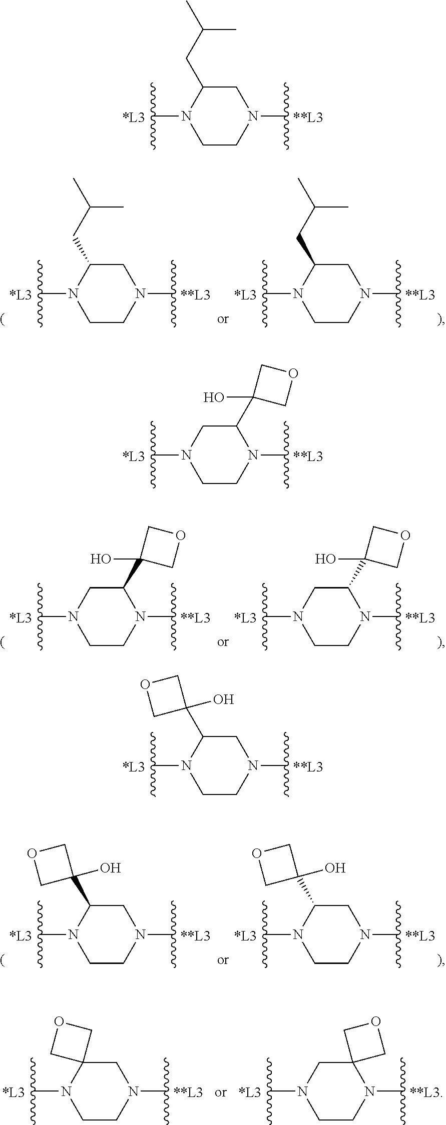

21.-24. (canceled)

25. The compound of claim 1, wherein 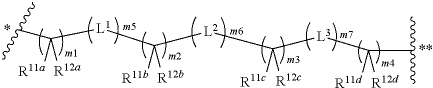
moiety is selected from

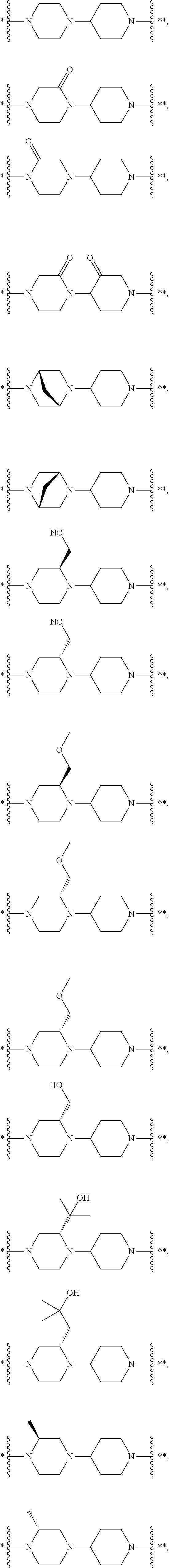

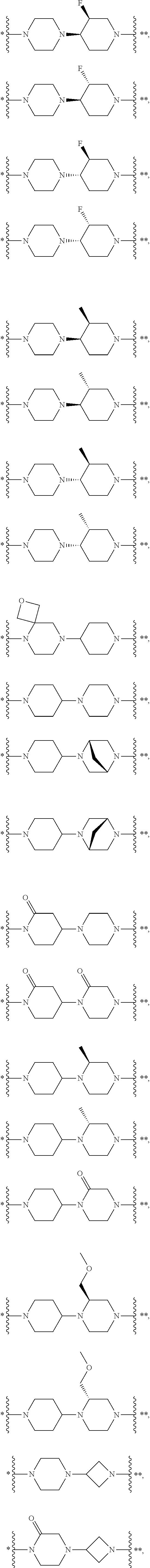

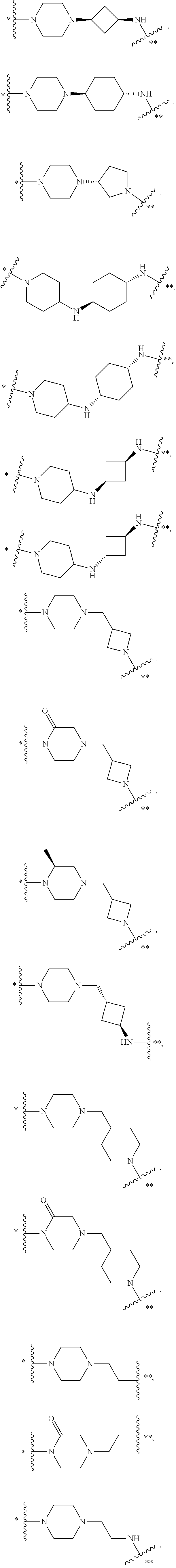

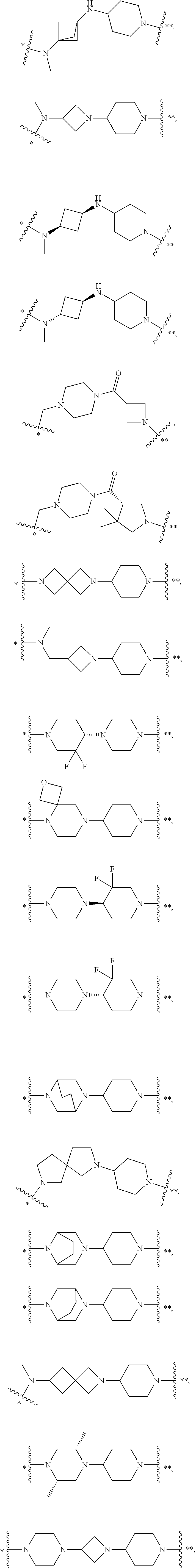

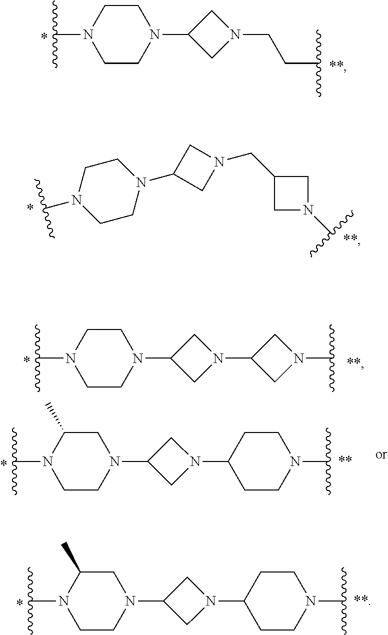

26. (canceled)

27. The compound of claim 1, wherein
X⁷ is independently selected from —CH, —C(CH₃), or N;
at most one of Z¹ and Z² is N;
R^{z} is selected from H, —CH₃, —C₂H₅, F, CH₂F, —CHF₂, —CF₃—OCH₃, —OC₂H₅, —C₃H₇, —OCH₂F, —OCHF₂, —OCH₂CF₃, —OCF₃, —SCF₃, —CF₃ or —CH(OH)CH₃;
R¹³ is each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₂, —OCF₃, —SCF₃, or phenyl.

28. (canceled)

29. The compound of claim 1, wherein [symbol] is selected from

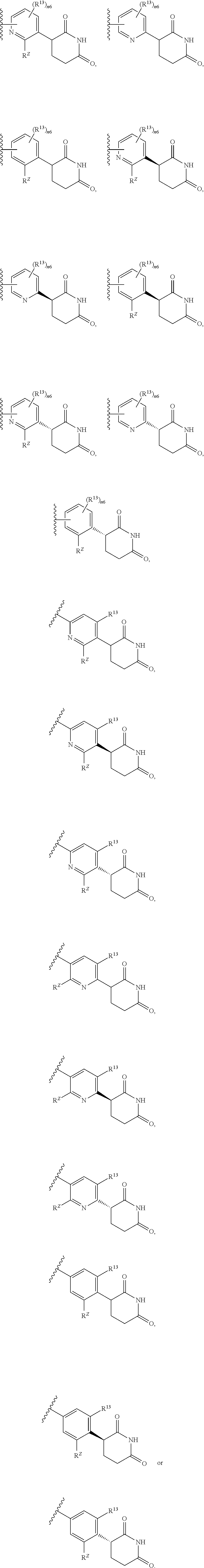

30.-32. (canceled)

33. The compound of claim 1, where [symbol] is

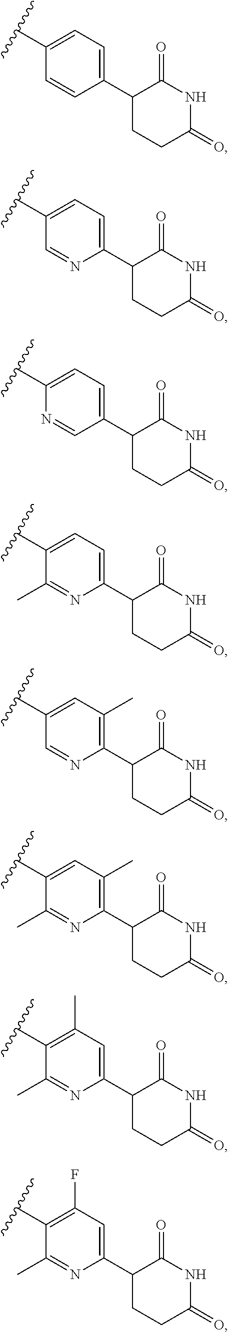

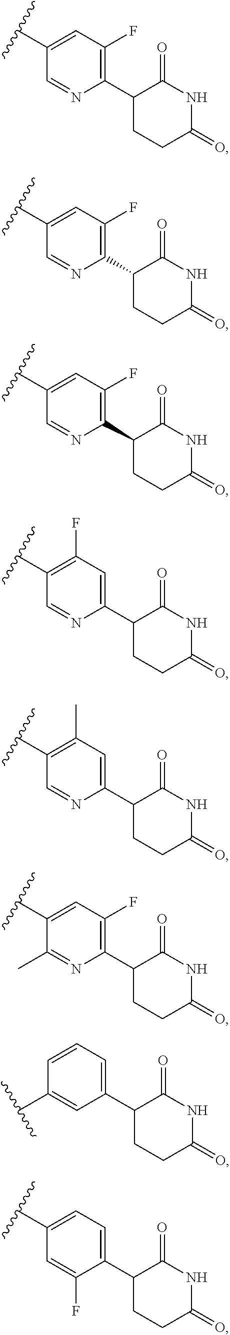

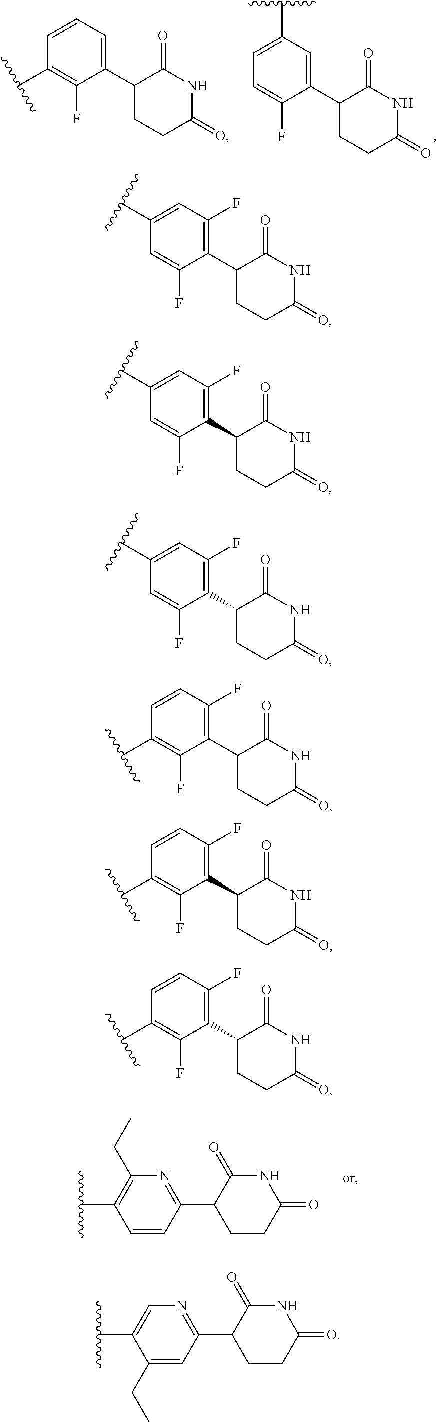

34. (canceled)

35. A compound, wherein the compound is selected from 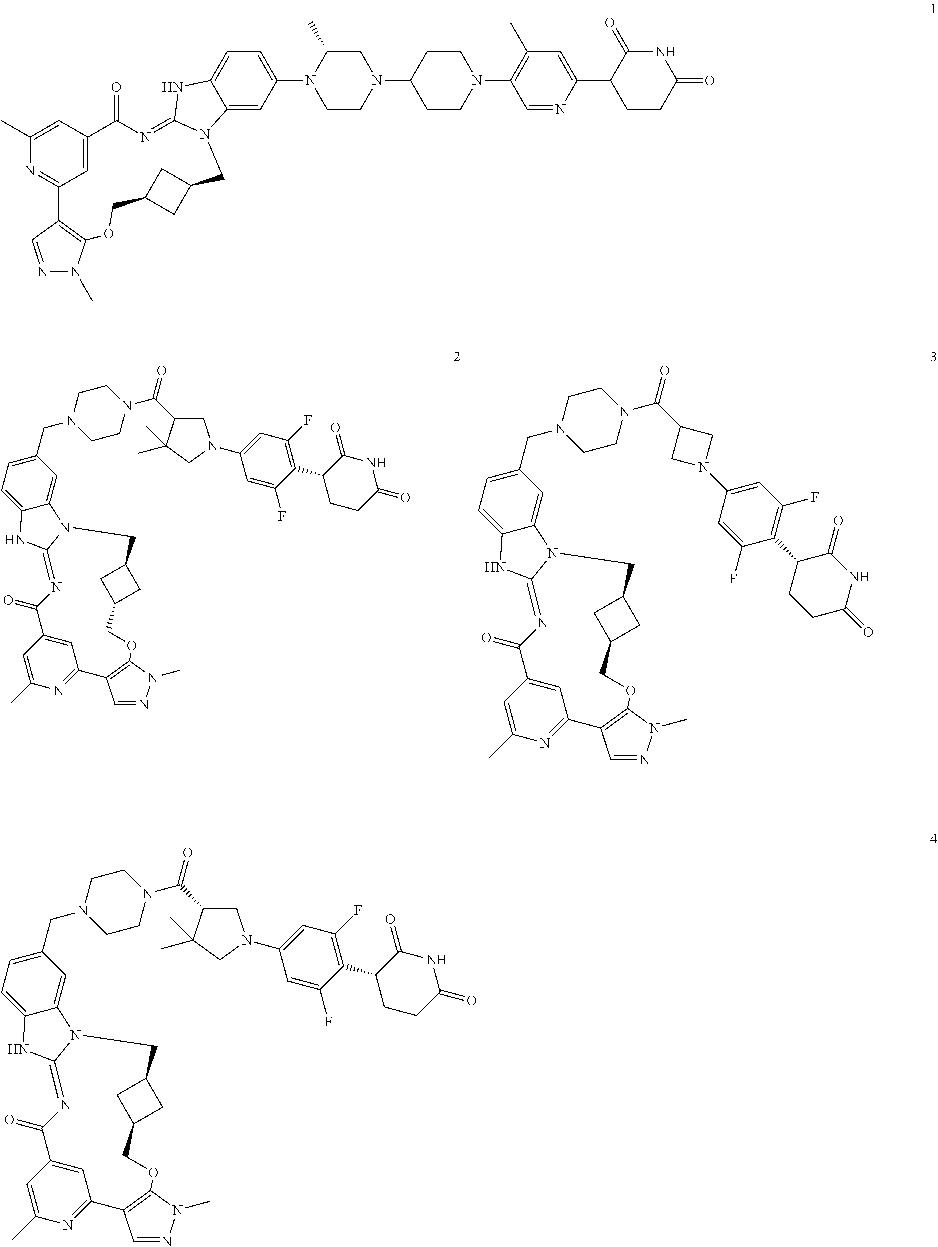 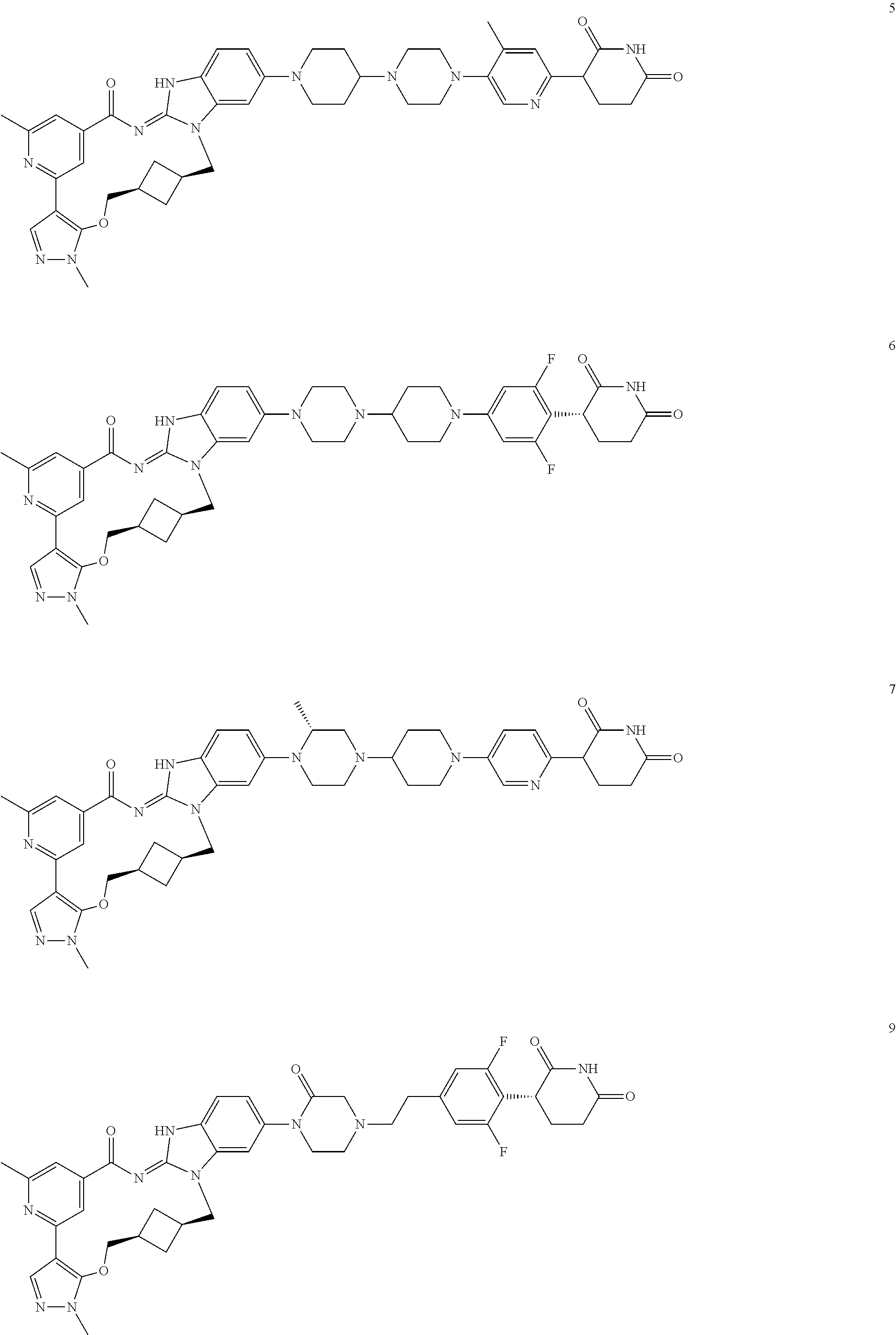 [SMILES: Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6C)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C[C@H]4C)cc23)C1 ; Cc1cc2cc(n1)-c1cnn(C)c1OC[C@H]1C[C@@H](CN3/C(=N/C2=O)Nc2ccc(N4CCN(CCc5cc(F)c([C@H]6CCC(=O)NC6=O)c(F)c5)CC4=O)cc23)C1] 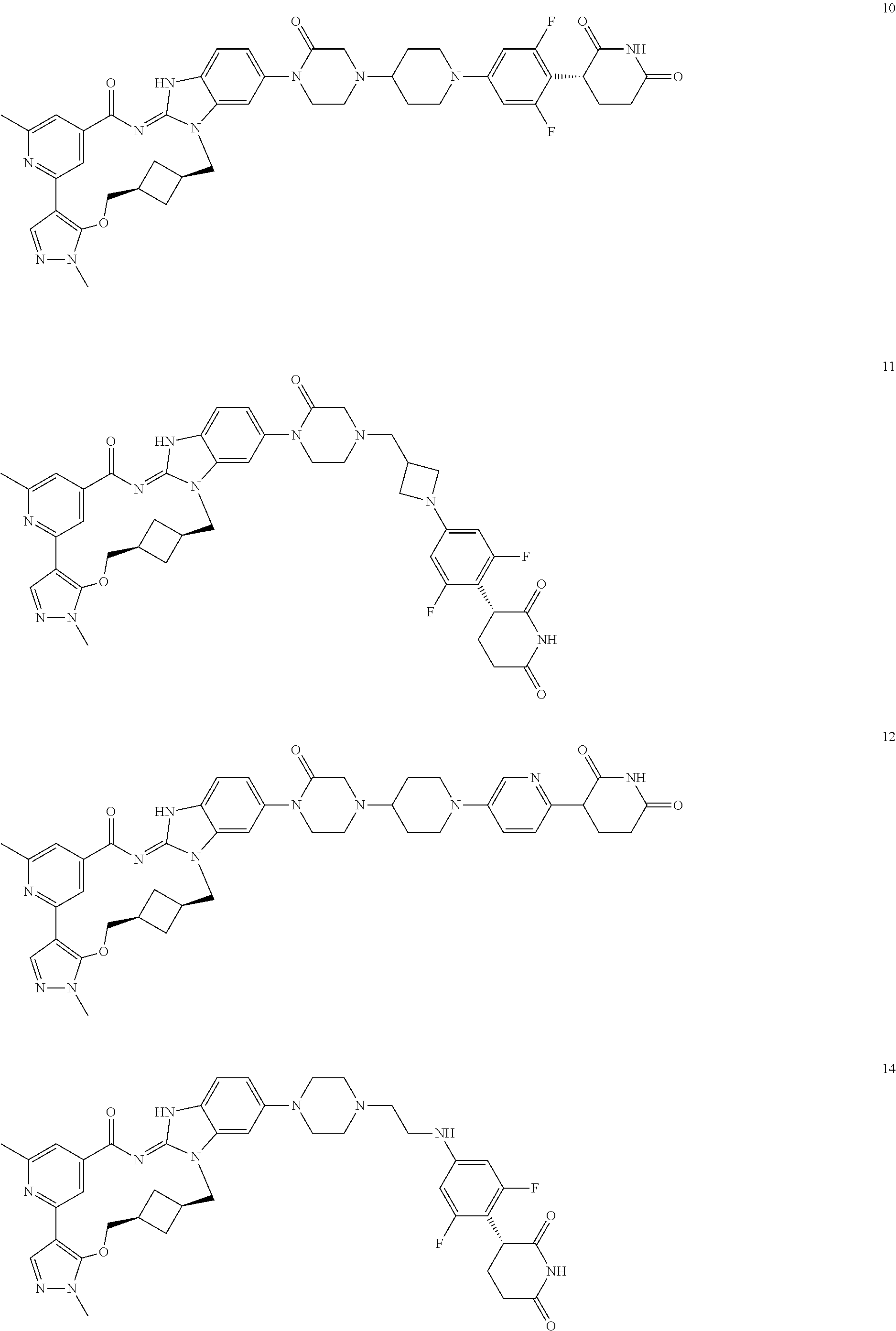 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)CC4=O)cc23)C1 ; Cc1cc2cc(n1)-c1cnn(C)c1OC[C@H]1C[C@@H](CN3/C(=N/C2=O)Nc2ccc(N4CCN(CC5CN(c6cc(F)c([C@H]7CCC(=O)NC7=O)c(F)c6)C5)CC4=O)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)CC4=O)cc23)C1 ; Cc1cc2cc(n1)-c1cnn(C)c1OC[C@H]1C[C@@H](CN3/C(=N/C2=O)Nc2ccc(N4CCN(CCNc5cc(F)c([C@H]6CCC(=O)NC6=O)c(F)c5)CC4)cc23)C1] 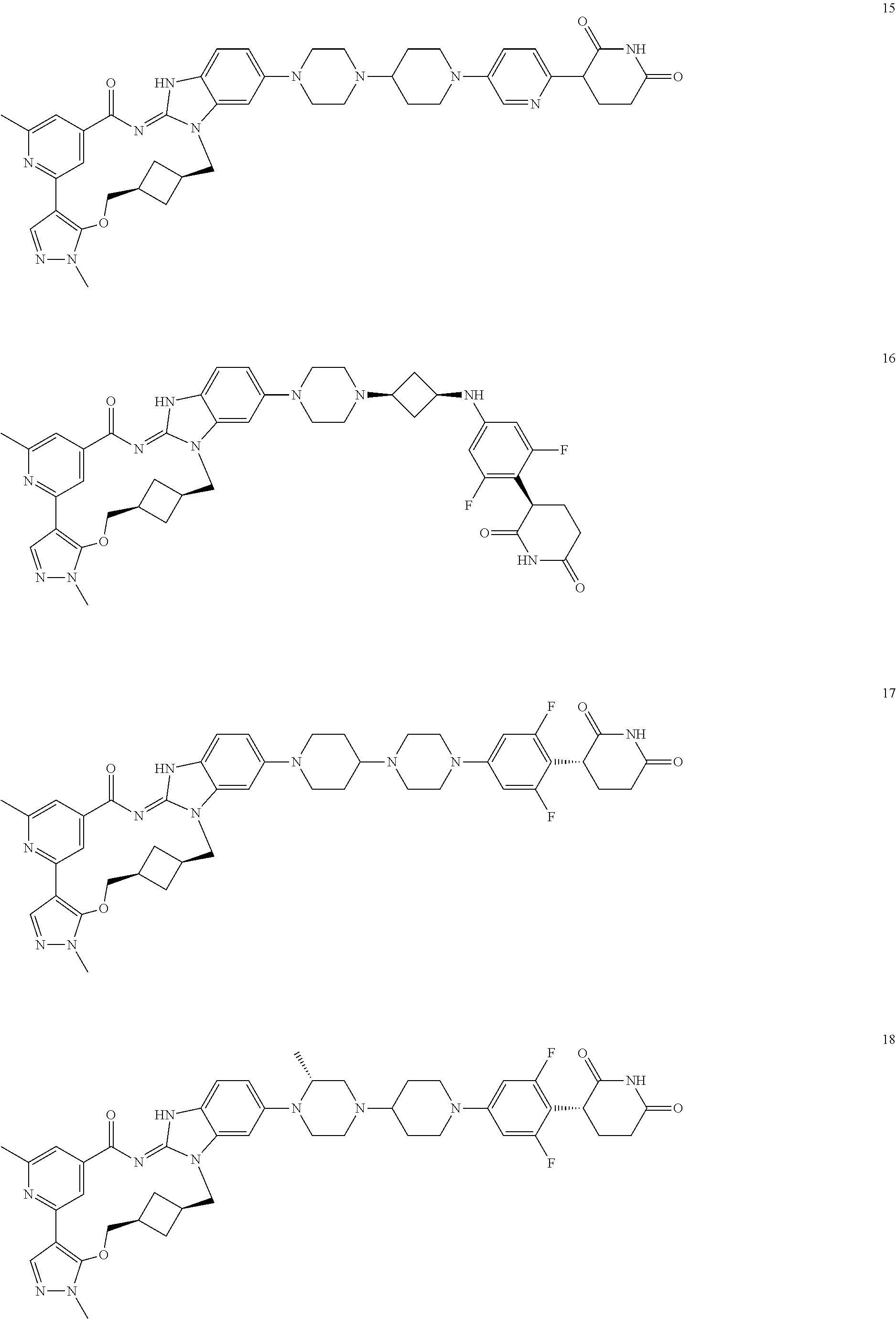 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)CC4)cc23)C1 ; Cc1cc2cc(n1)-c1cnn(C)c1OC[C@H]1C[C@@H](CN3/C(=N/C2=O)Nc2ccc(N4CCN([C@H]5C[C@@H](Nc6cc(F)c([C@H]7CCC(=O)NC7=O)c(F)c6)C5)CC4)cc23)C1 ; Cc1cc2cc(n1)-c1cnn(C)c1OC[C@H]1C[C@@H](CN3/C(=N/C2=O)Nc2ccc(N4CCC(N5CCN(c6cc(F)c([C@H]7CCC(=O)NC7=O)c(F)c6)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)C[C@H]4C)cc23)C1] 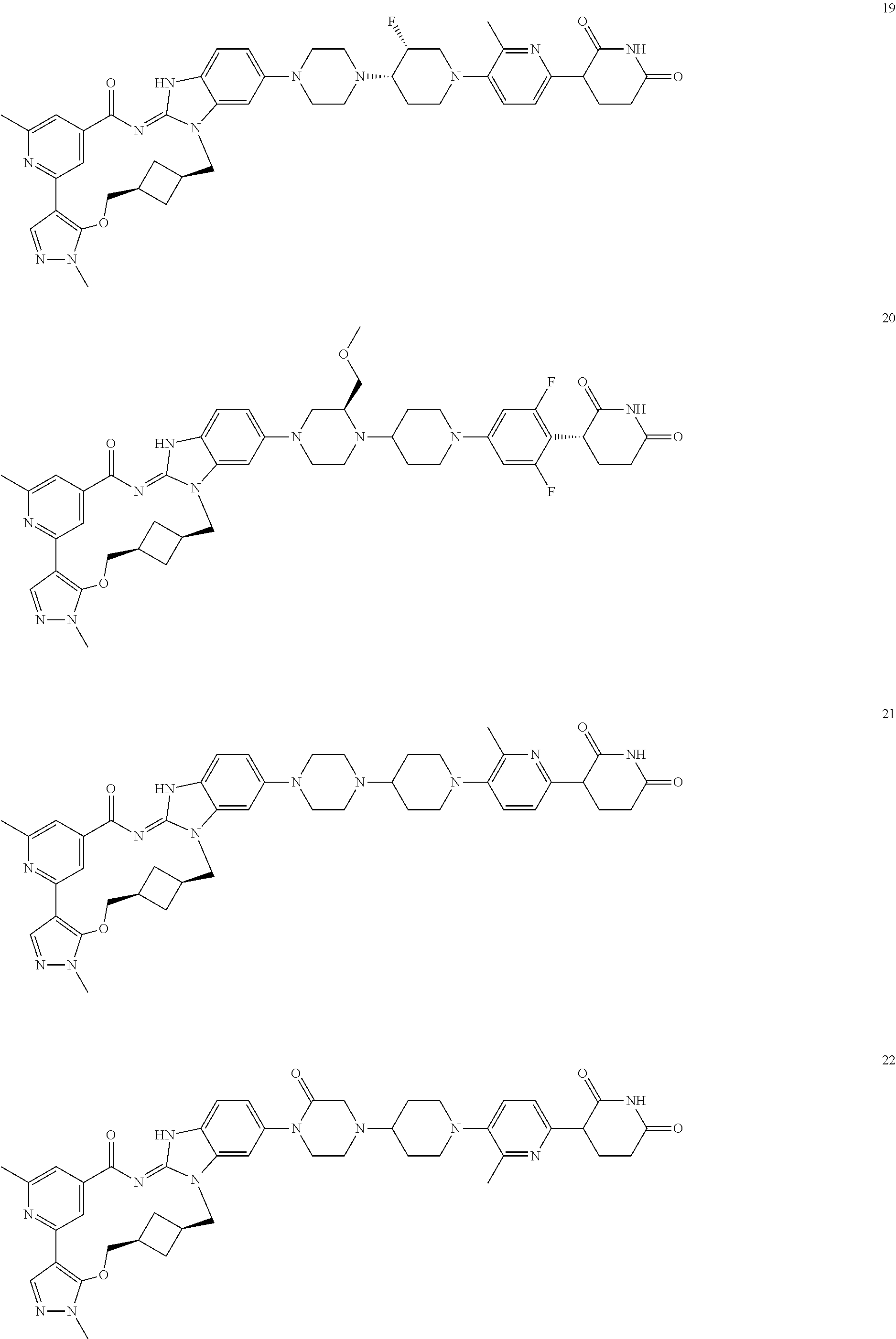 [SMILES: Cc1cc2cc(n1)-c1cnn(C)c1OC[C@H]1C[C@@H](CN3/C(=N/C2=O)Nc2ccc(N4CCN([C@H]5CCN(c6ccc(C7CCC(=O)NC7=O)nc6C)C[C@H]5F)CC4)cc23)C1 ; COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2cc(F)c([C@H]3CCC(=O)NC3=O)c(F)c2)CC1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC4=O)cc23)C1] 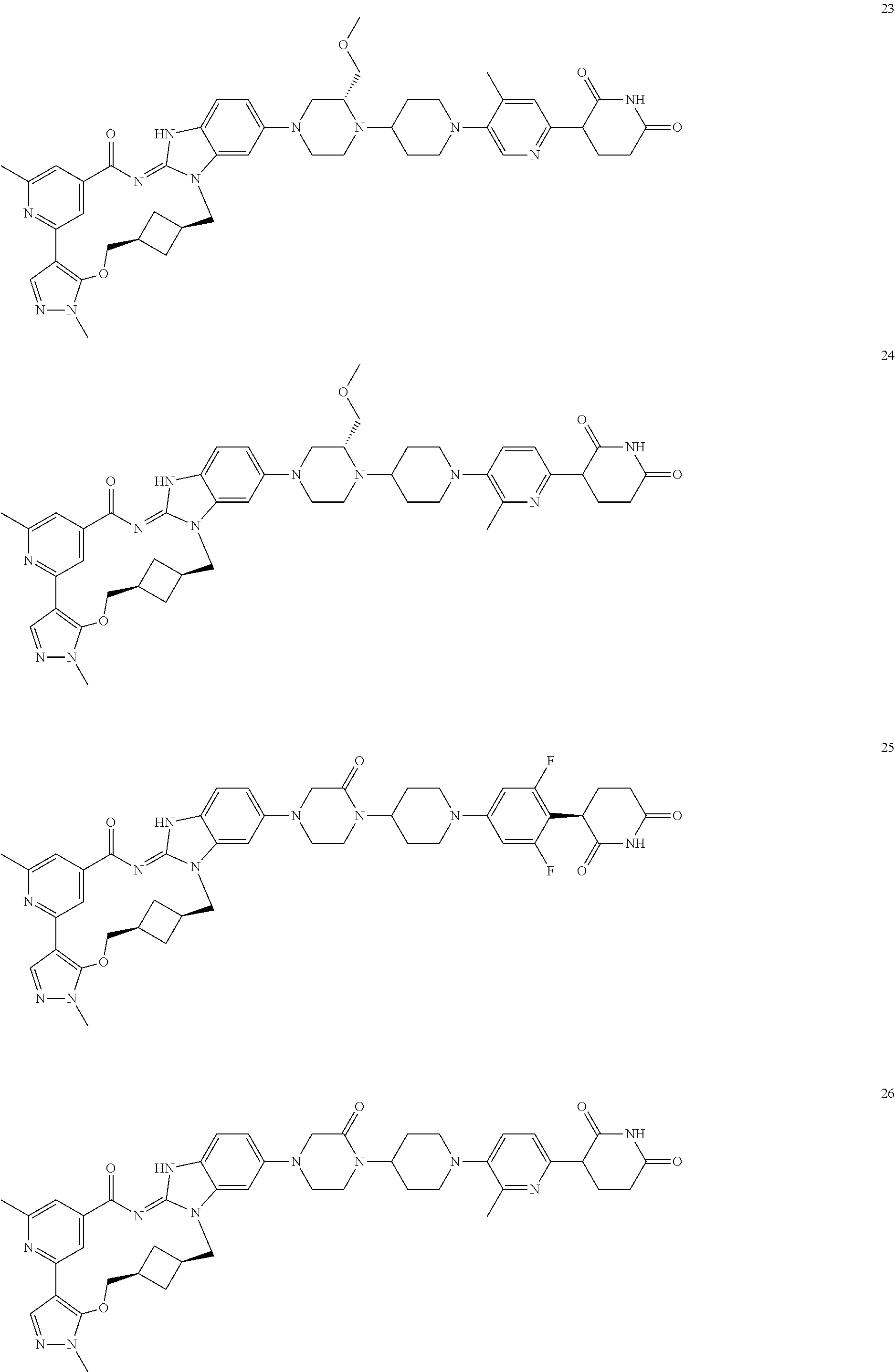 [SMILES: COC[C@H]1CN(c2ccc3c(c2)N2C[C@H]4C[C@@H](COc5c(cnn5C)-c5cc(cc(C)n5)C(=O)/N=C/2N3)C4)CCN1C1CCN(c2cnc(C3CCC(=O)NC3=O)cc2C)CC1 ; COC[C@H]1CN(c2ccc3c(c2)N2C[C@H]4C[C@@H](COc5c(cnn5C)-c5cc(cc(C)n5)C(=O)/N=C/2N3)C4)CCN1C1CCN(c2ccc(C3CCC(=O)NC3=O)nc2C)CC1 ; Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)C(=O)C4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C(=O)C4)cc23)C1] 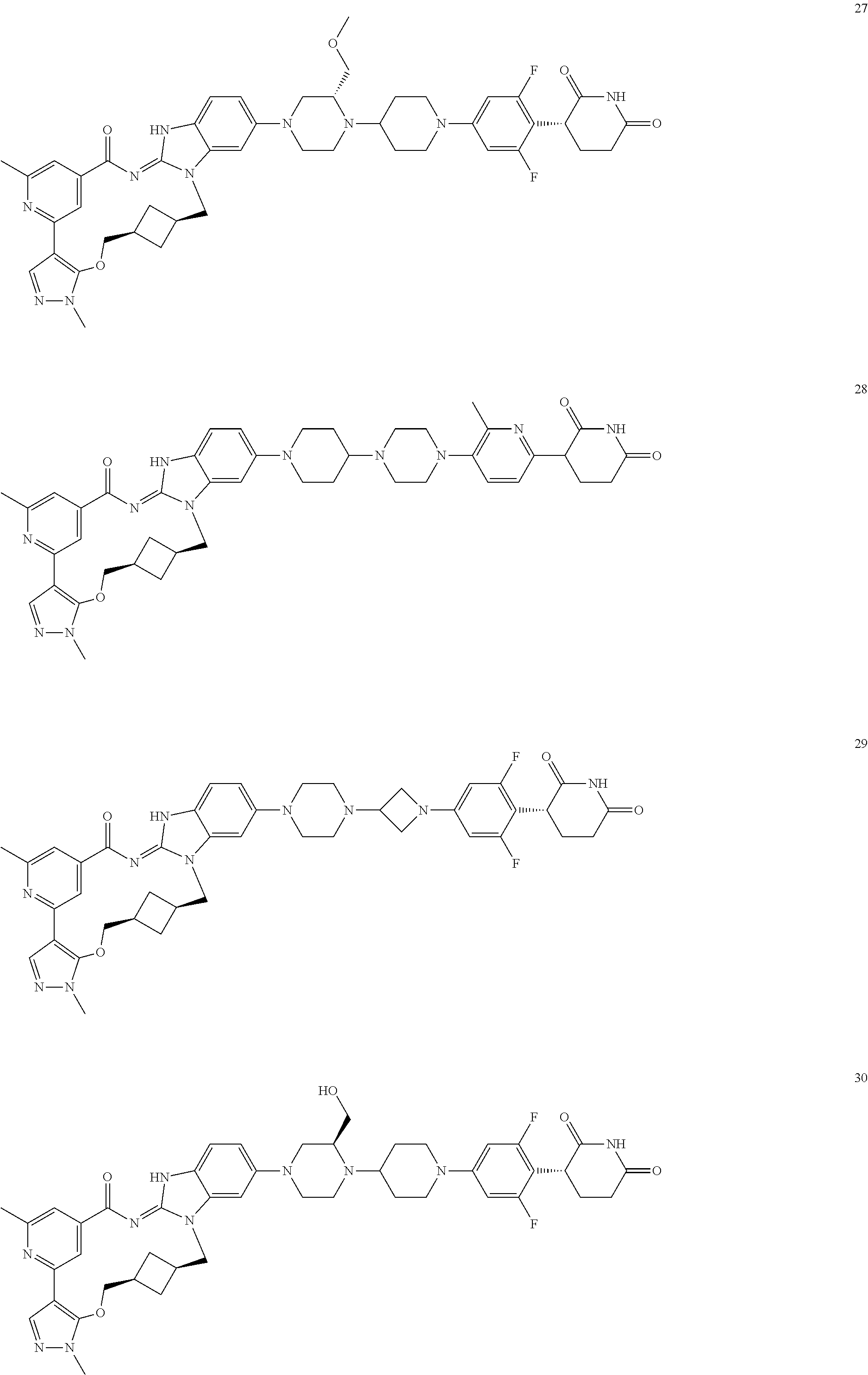 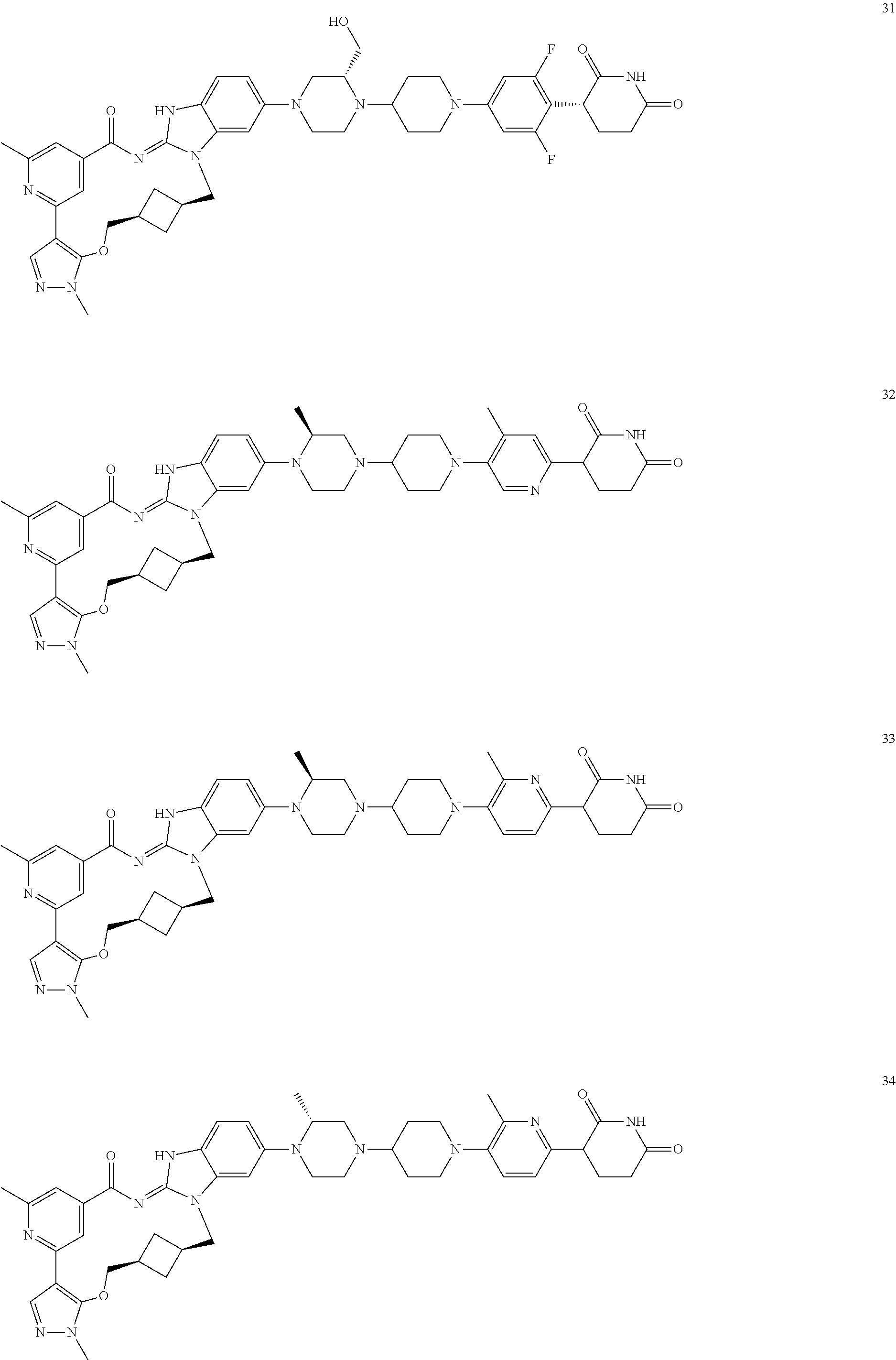 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)[C@@H](CO)C4)cc23)C1 ; Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)C[C@@H]4C)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C[C@@H]4C)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)C[C@H]4C)cc23)C1] 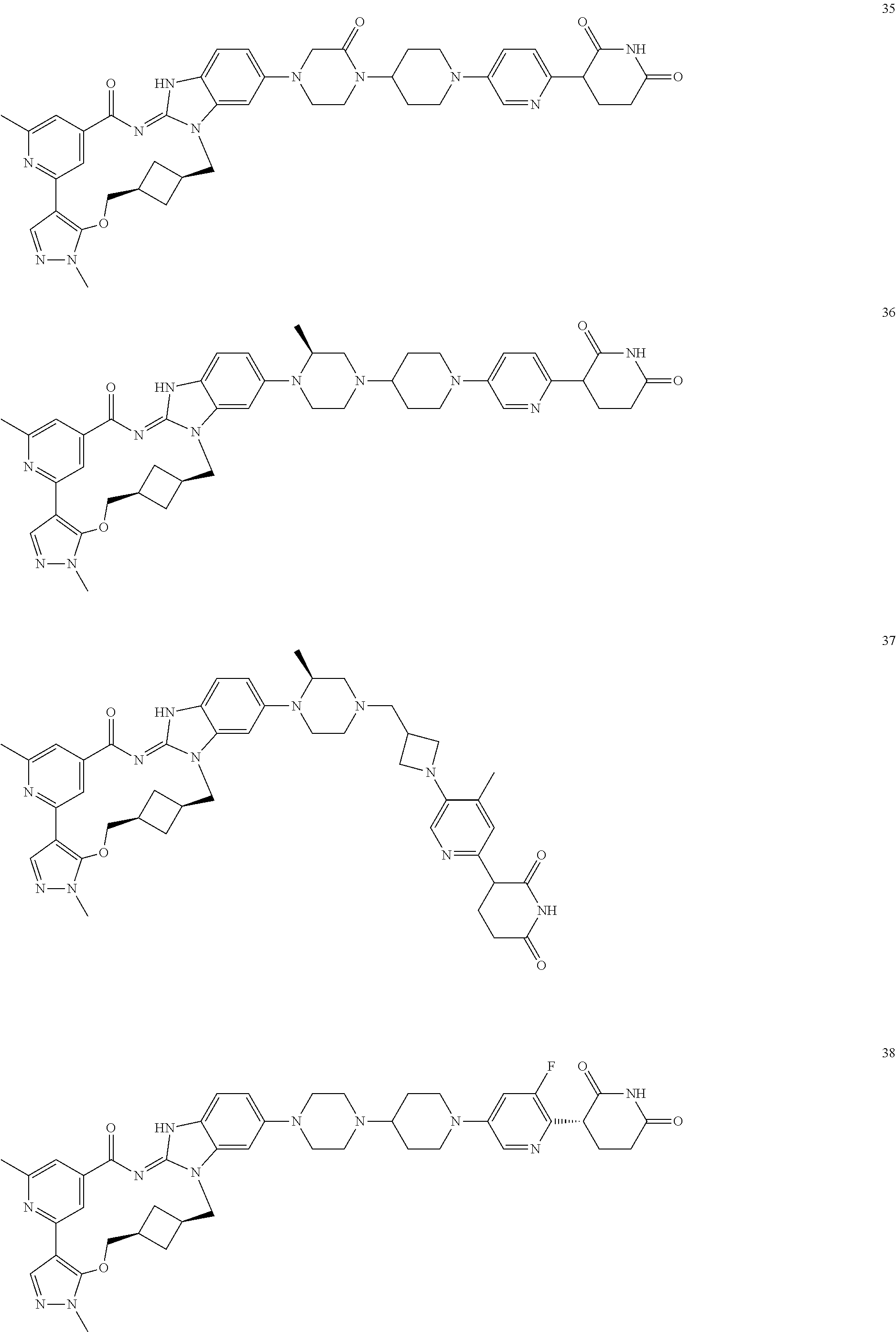 [SMILES: Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C(=O)C4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6)CC5)C[C@@H]4C)cc23)C1 ; Cc1cc2cc(n1)-c1cnn(C)c1OC[C@H]1C[C@@H](CN3/C(=N/C2=O)Nc2ccc(N4CCN(CC5CN(c6cnc(C7CCC(=O)NC7=O)cc6C)C5)C[C@@H]4C)cc23)C1 ; Cc1cc2cc(n1)-c1cnn(C)c1OC[C@H]1C[C@@H](CN3/C(=N/C2=O)Nc2ccc(N4CCN(C5CCN(c6cnc([C@H]7CCC(=O)NC7=O)c(F)c6)CC5)CC4)cc23)C1] 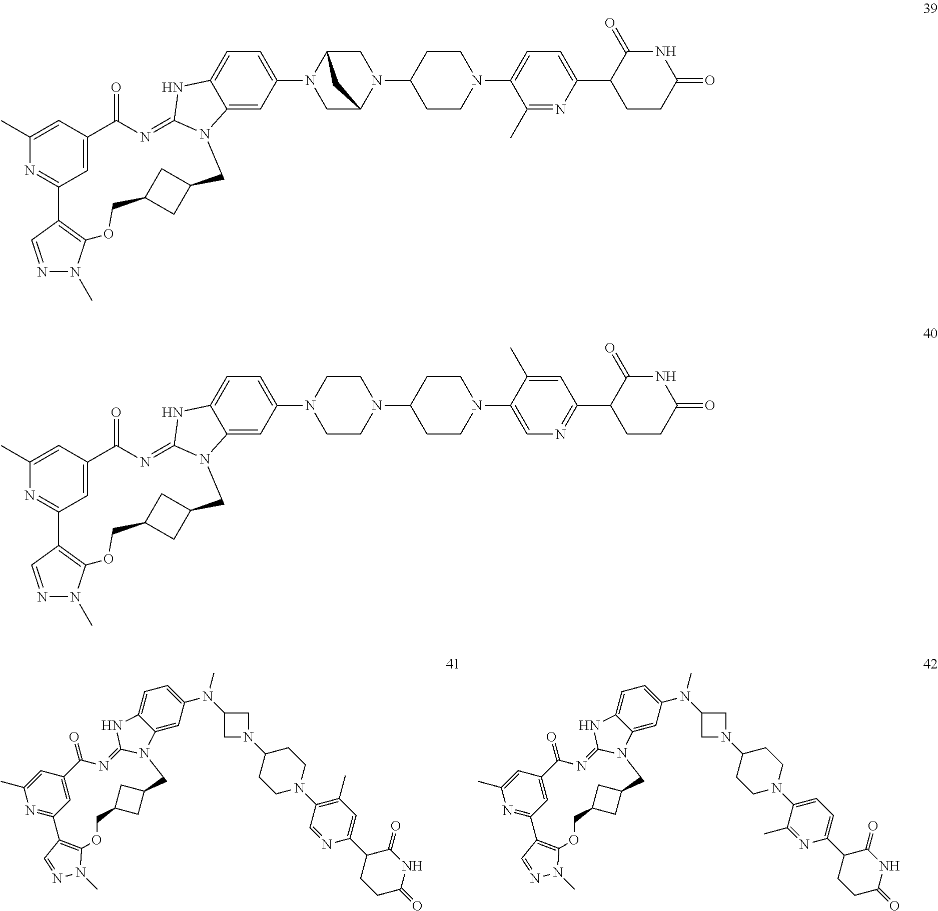 [SMILES: Cc1cc2cc(n1)-c1cnn(C)c1OC[C@H]1C[C@@H](CN3/C(=N/C2=O)Nc2ccc(N4C[C@@H]5C[C@H]4CN5C4CCN(c5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)CC4)cc23)C1 ; Cc1cc2cc(n1)-c1cnn(C)c1OC[C@H]1C[C@@H](CN3/C(=N/C2=O)Nc2ccc(N(C)C4CN(C5CCN(c6cnc(C7CCC(=O)NC7=O)cc6C)CC5)C4)cc23)C1 ; Cc1cc2cc(n1)-c1cnn(C)c1OC[C@H]1C[C@@H](CN3/C(=N/C2=O)Nc2ccc(N(C)C4CN(C5CCN(c6ccc(C7CCC(=O)NC7=O)nc6C)CC5)C4)cc23)C1] 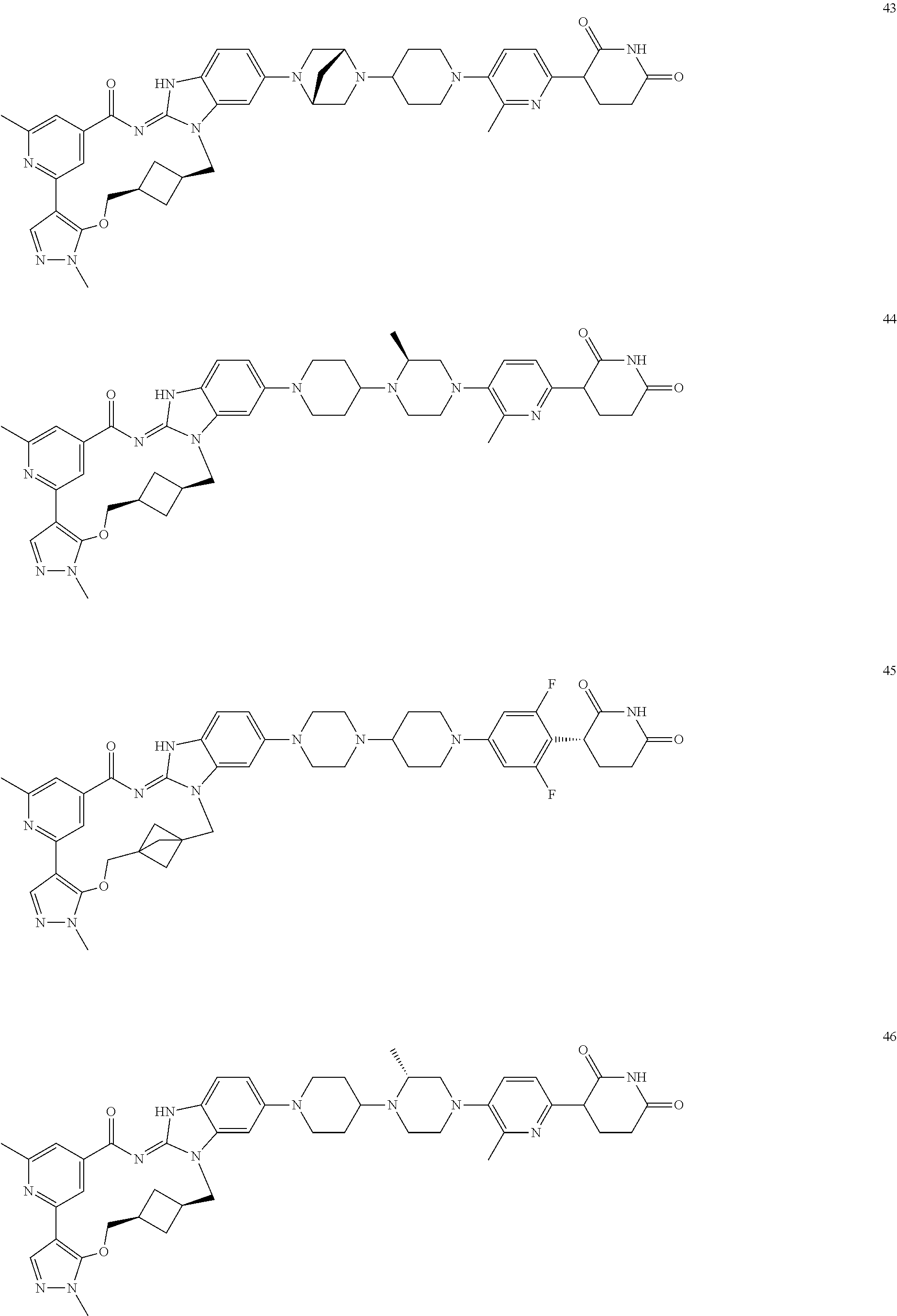 [SMILES: Cc1cc2cc(n1)-c1cnn(C)c1OC[C@H]1C[C@@H](CN3/C(=N/C2=O)Nc2ccc(N4C[C@H]5C[C@@H]4CN5C4CCN(c5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)C[C@@H]5C)CC4)cc23)C1 ; Cc1cc2cc(n1)-c1cnn(C)c1OCC13CC(CN4/C(=N/C2=O)Nc2ccc(N5CCN(C6CCN(c7cc(F)c([C@H]8CCC(=O)NC8=O)c(F)c7)CC6)CC5)cc24)(C1)C3 ; Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)C[C@H]5C)CC4)cc23)C1] 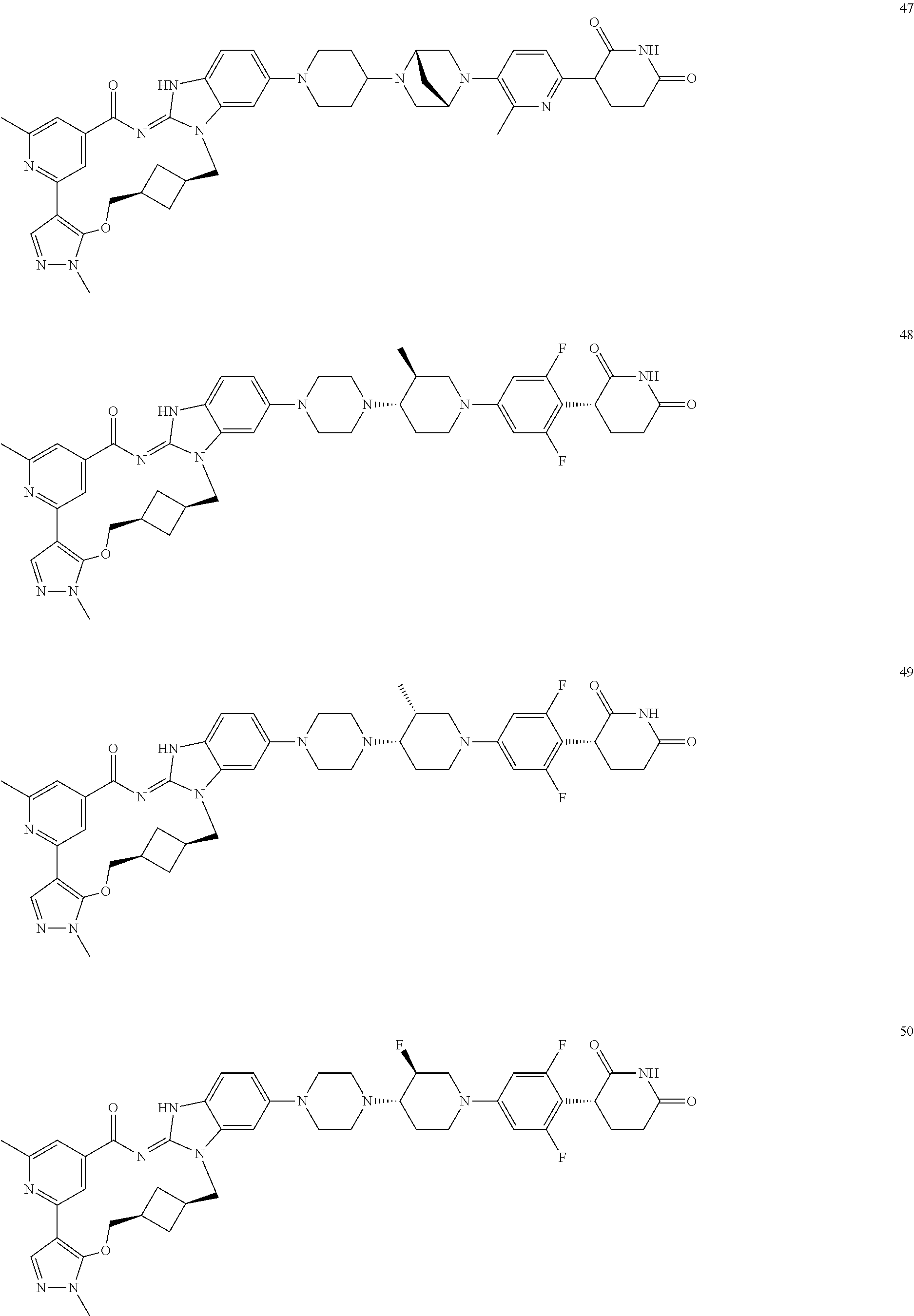 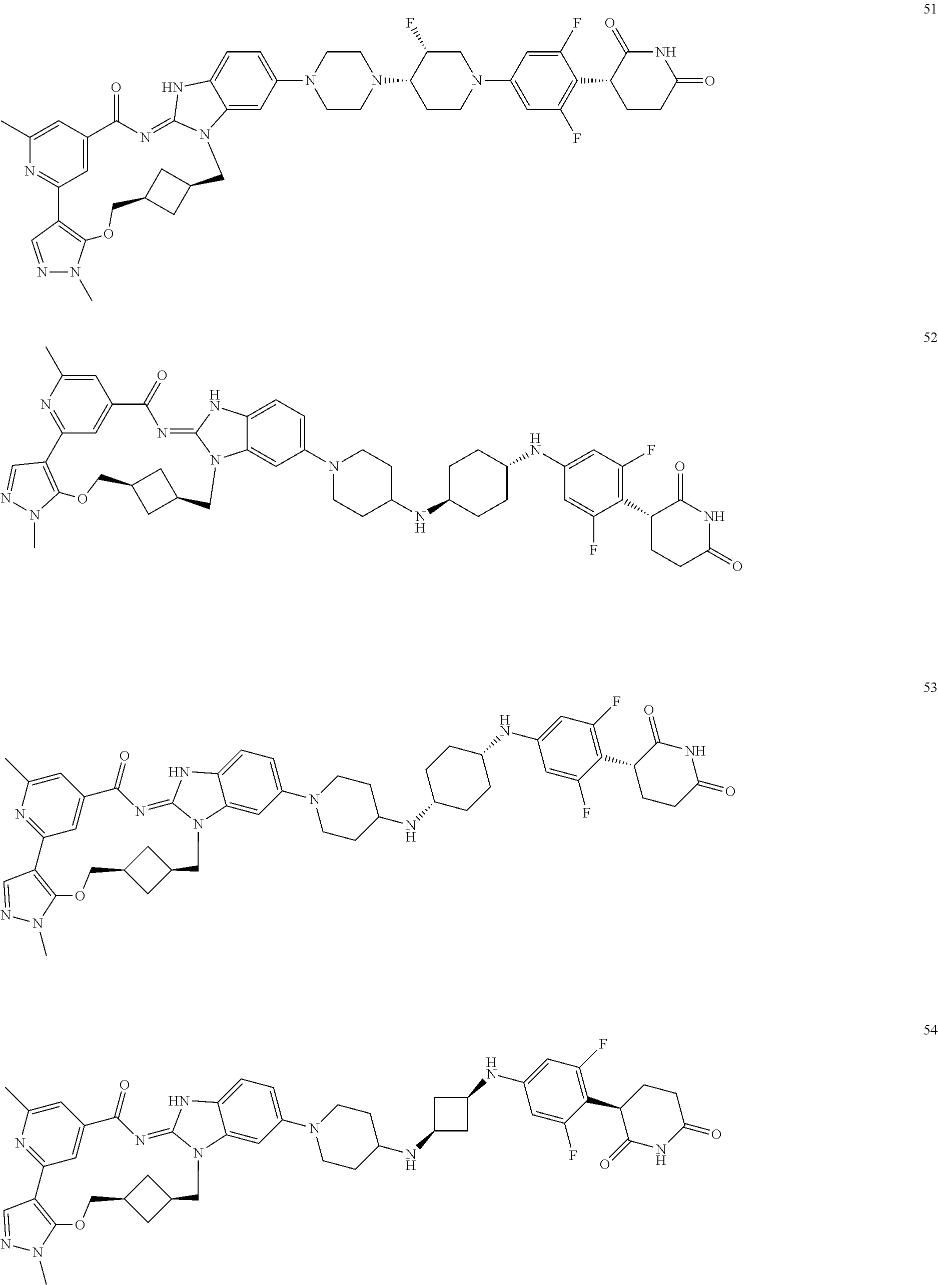 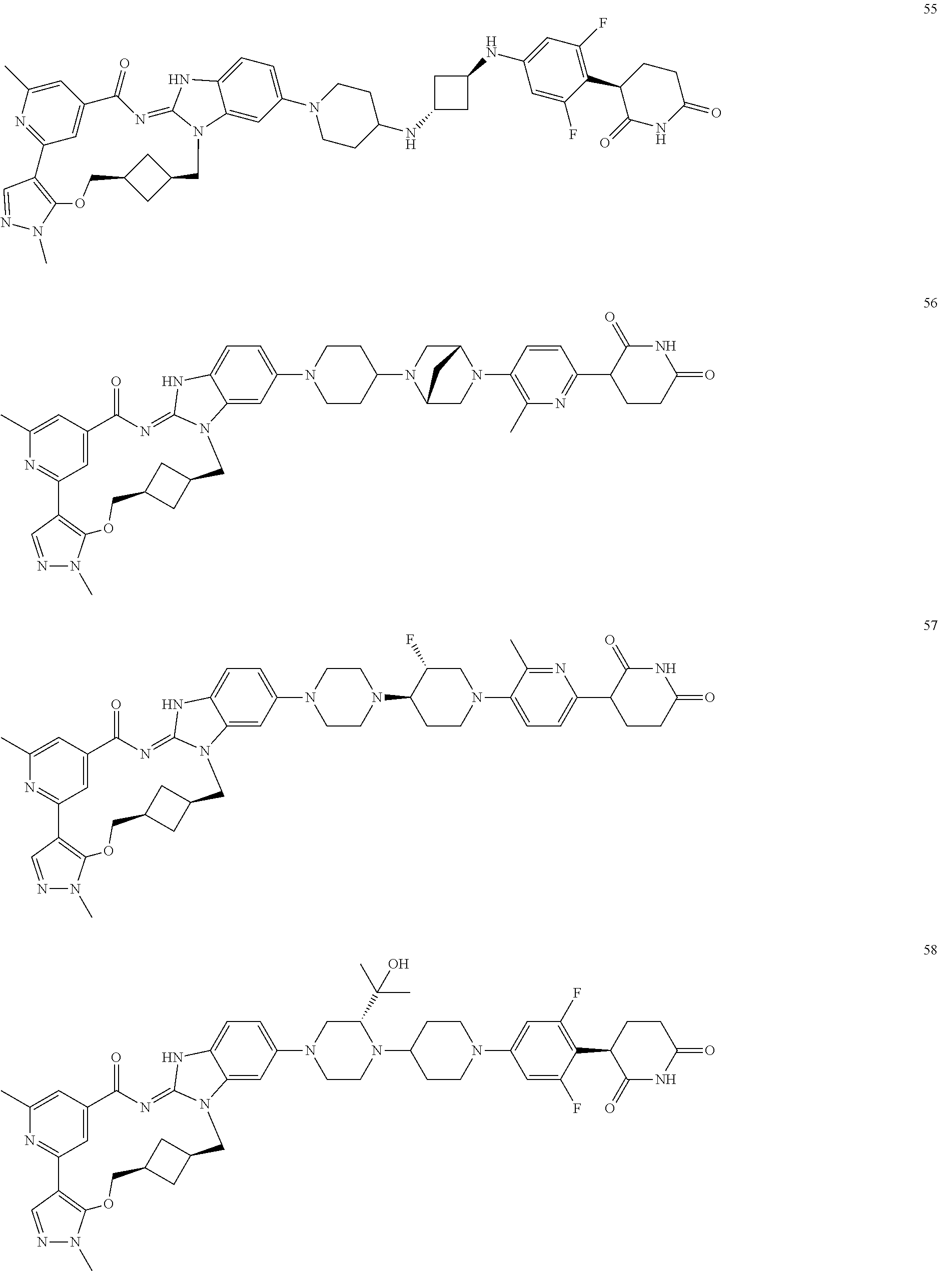 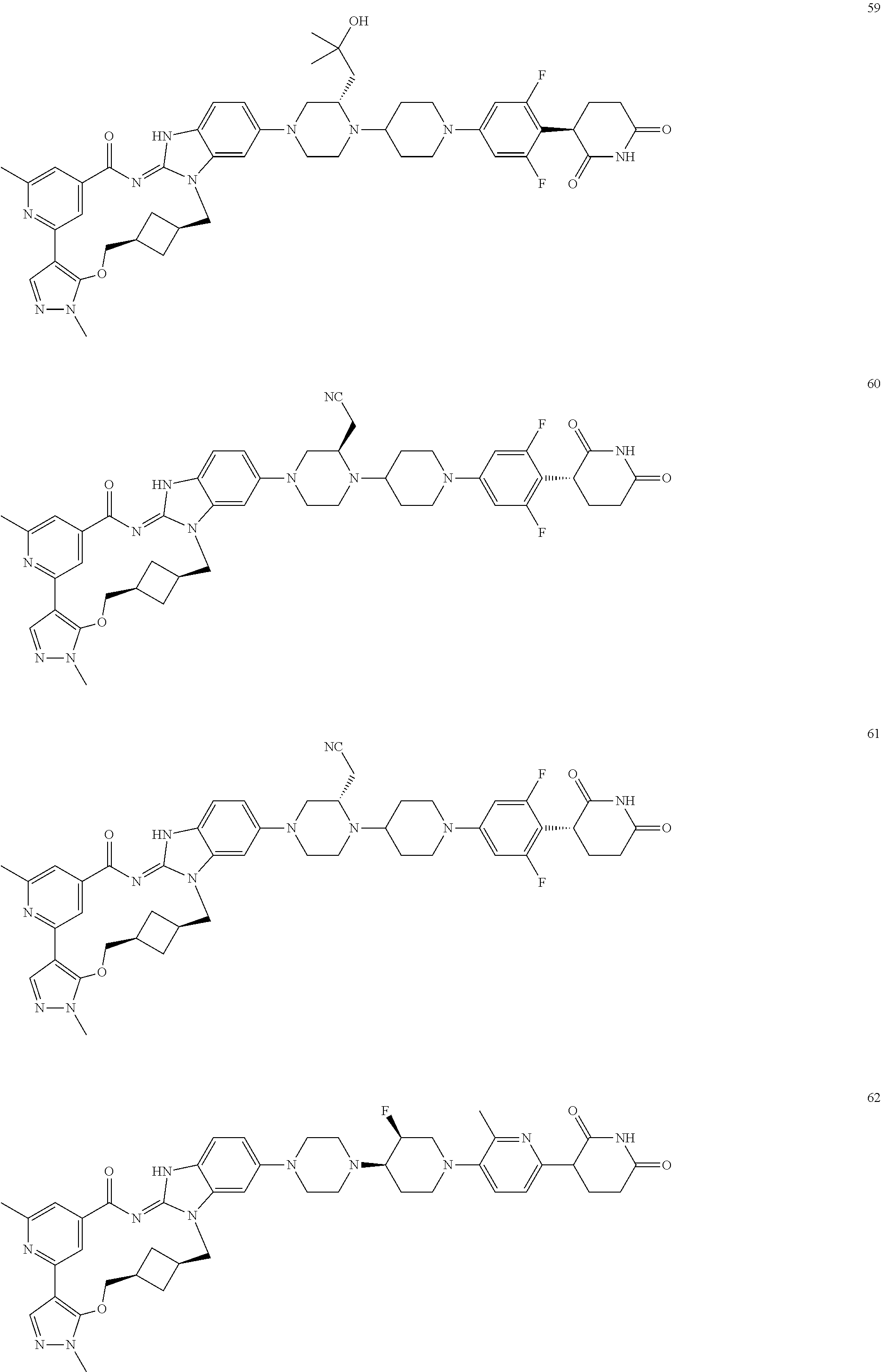 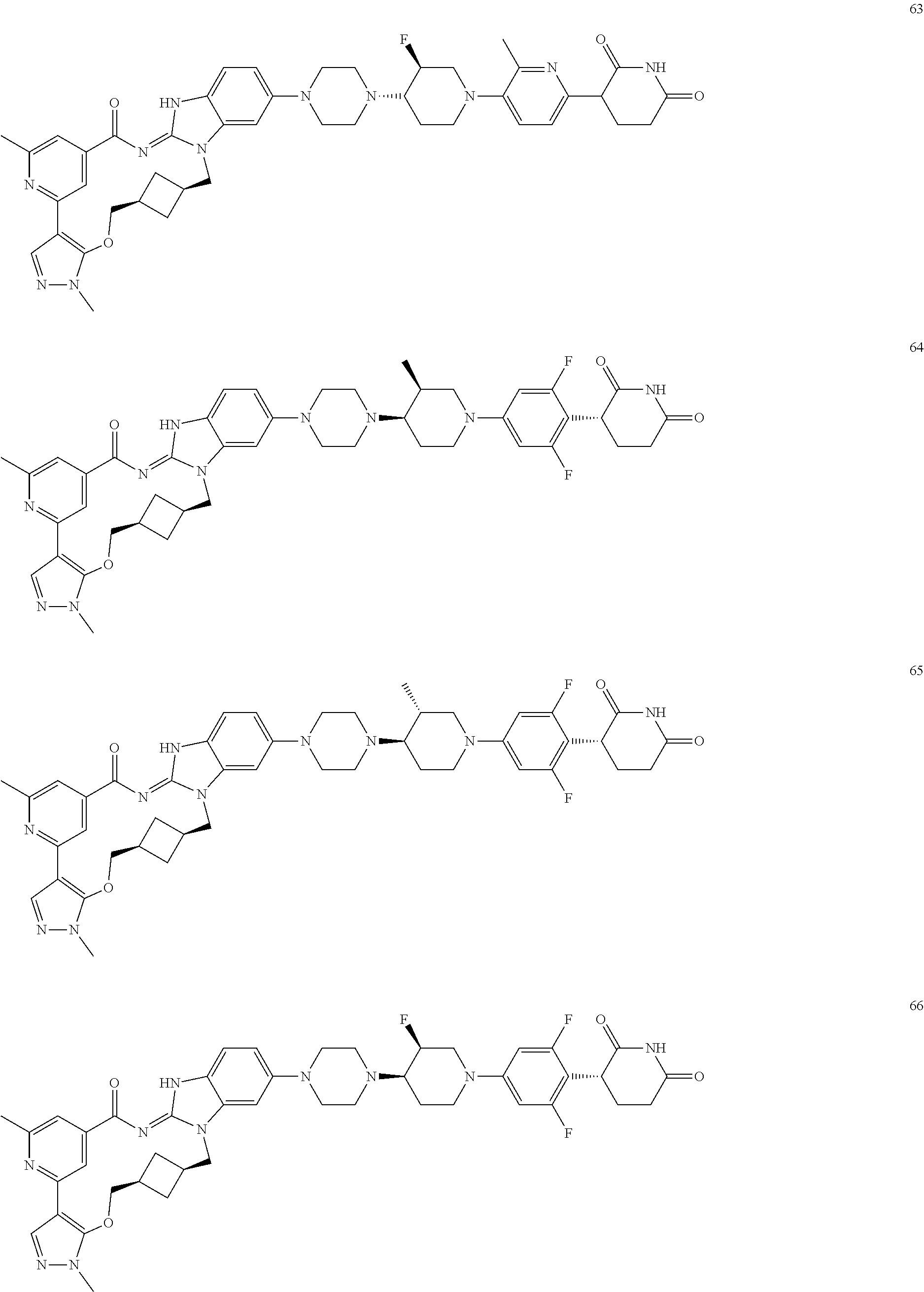 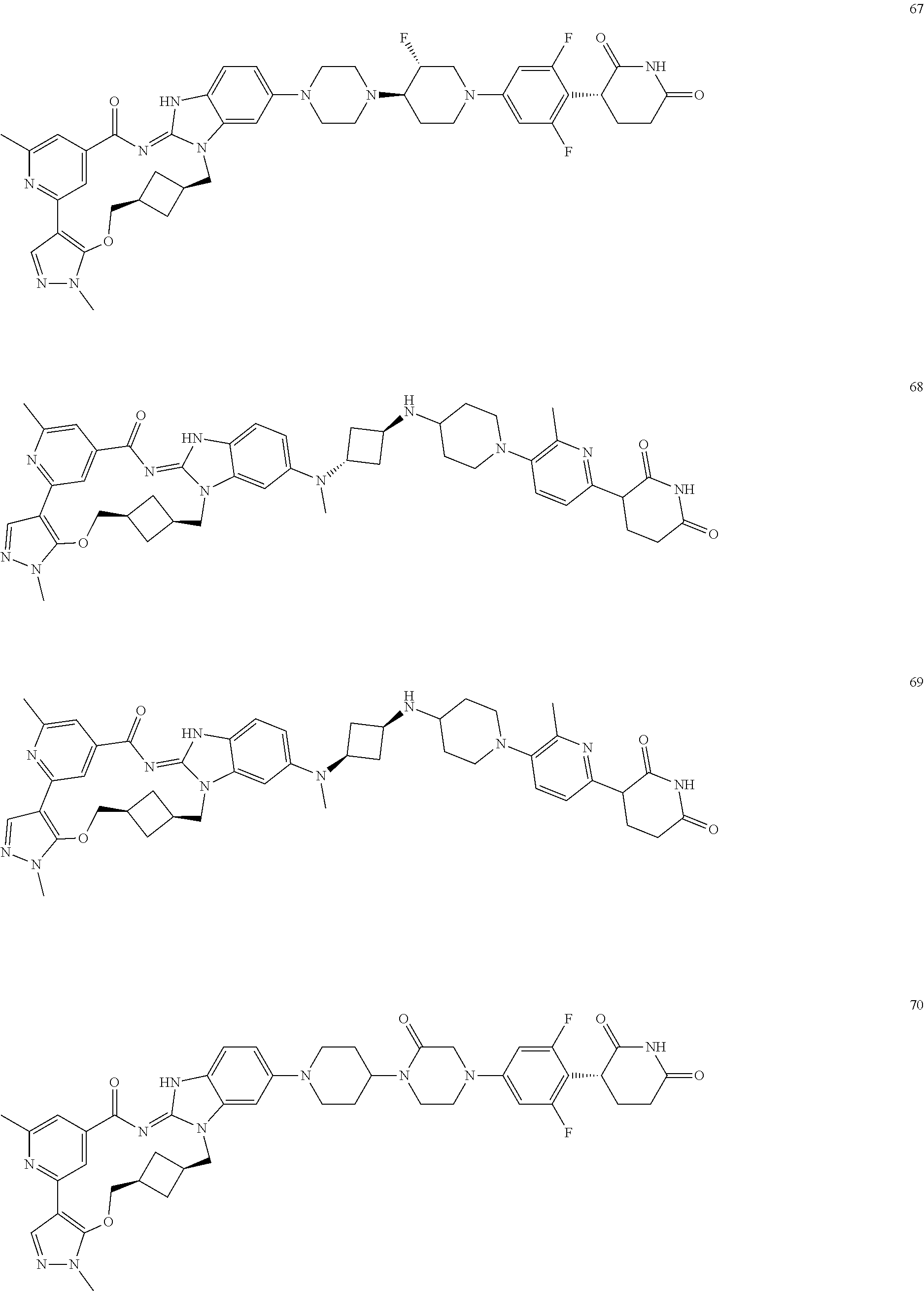 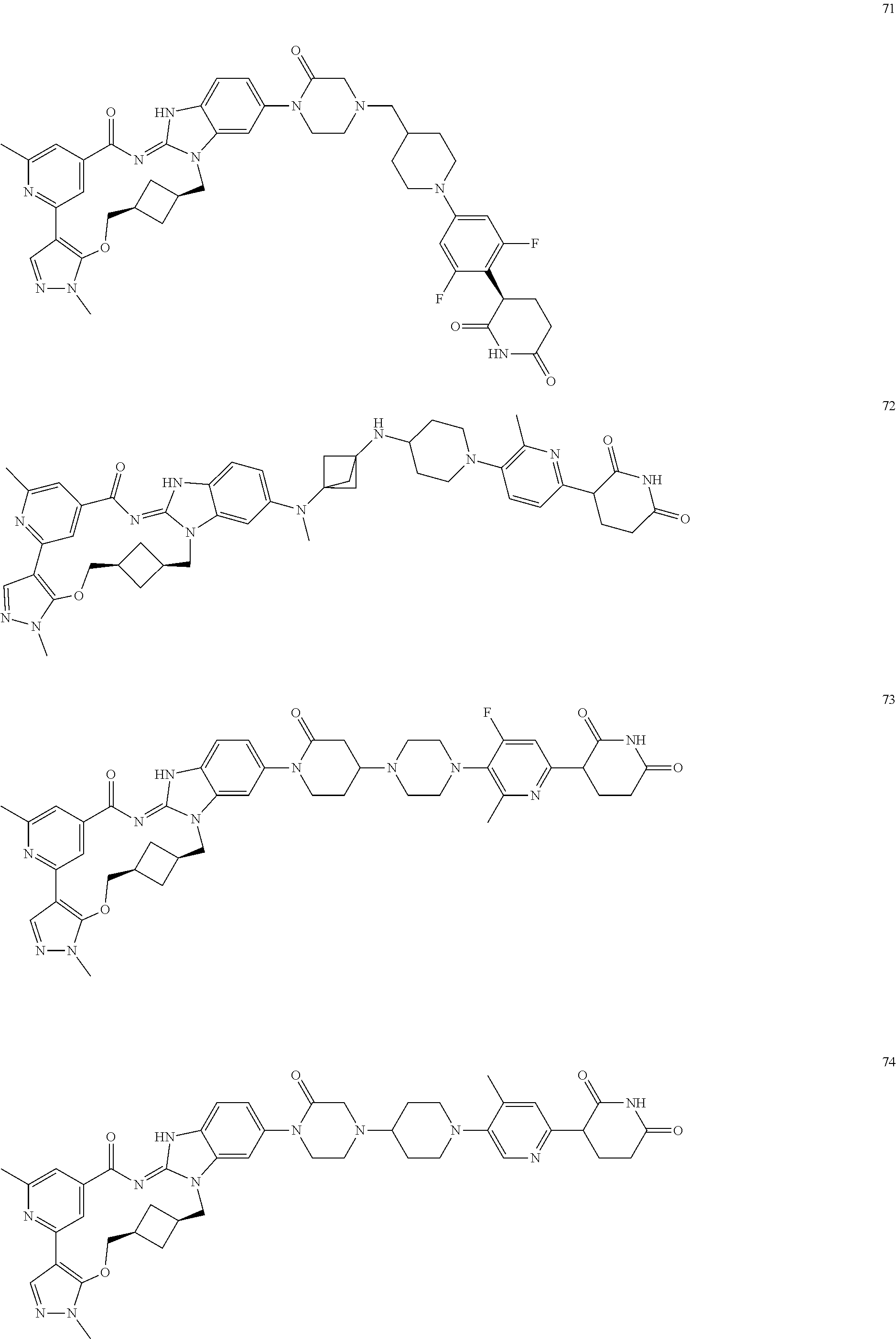 [SMILES: Cc1cc2cc(n1)-c1cnn(C)c1OC[C@H]1C[C@@H](CN3/C(=N/C2=O)Nc2ccc(N4CCN(CC5CCN(c6cc(F)c([C@H]7CCC(=O)NC7=O)c(F)c6)CC5)CC4=O)cc23)C1 ; Cc1cc2cc(n1)-c1cnn(C)c1OC[C@H]1C[C@@H](CN3/C(=N/C2=O)Nc2ccc(N(C)C45CC(NC6CCN(c7ccc(C8CCC(=O)NC8=O)nc7C)CC6)(C4)C5)cc23)C1 ; Cc1cc2cc(n1)-c1cnn(C)c1OC[C@H]1C[C@@H](CN3/C(=N/C2=O)Nc2ccc(N4CCC(N5CCN(c6c(F)cc(C7CCC(=O)NC7=O)nc6C)CC5)CC4=O)cc23)C1 ; Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)CC4=O)cc23)C1] 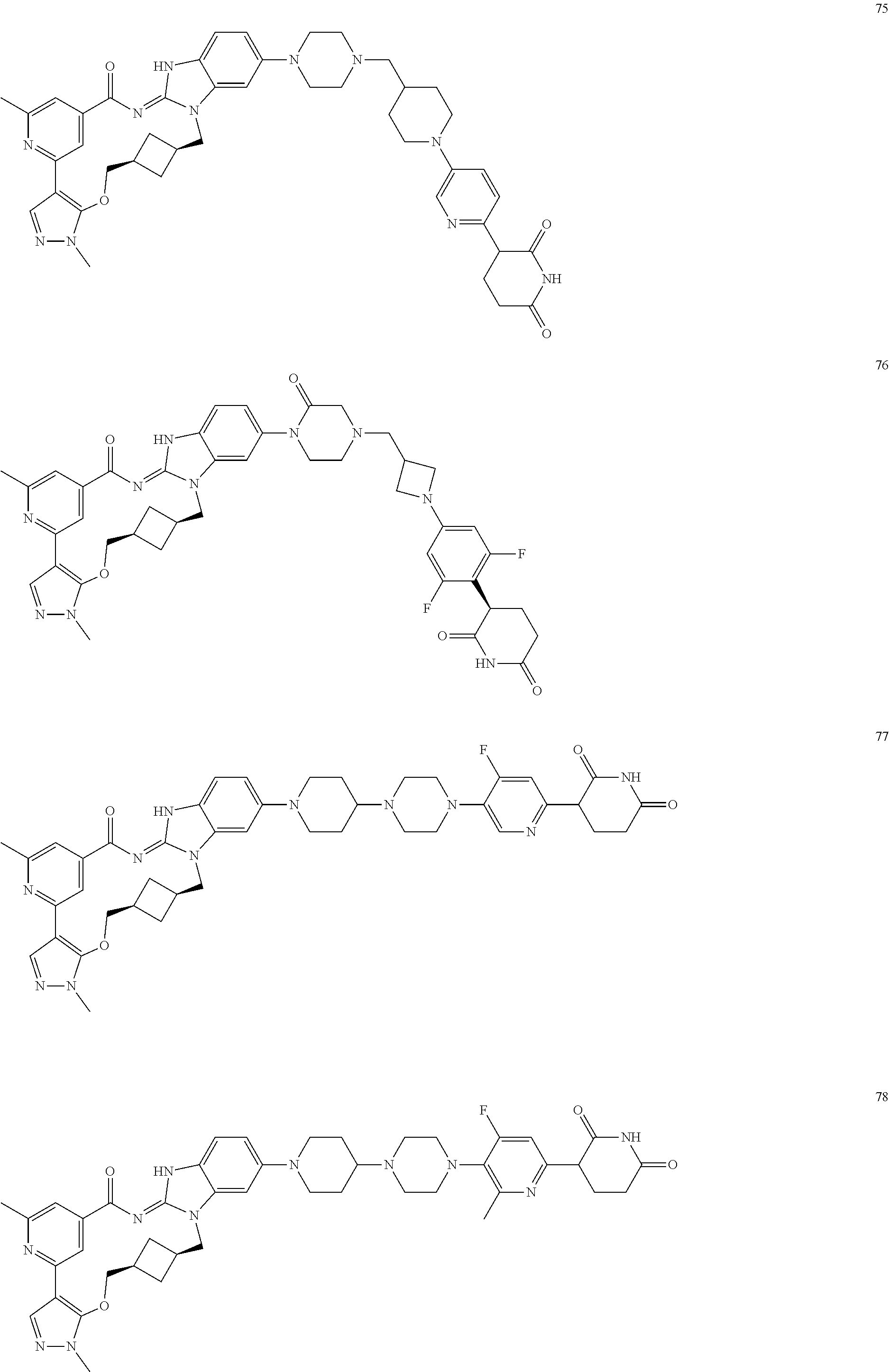 [SMILES: Cc1cc2cc(n1)-c1cnn(C)c1OC[C@H]1C[C@@H](CN3/C(=N/C2=O)Nc2ccc(N4CCN(CC5CCN(c6ccc(C7CCC(=O)NC7=O)nc6)CC5)CC4)cc23)C1 ; Cc1cc2cc(n1)-c1cnn(C)c1OC[C@H]1C[C@@H](CN3/C(=N/C2=O)Nc2ccc(N4CCN(CC5CN(c6cc(F)c([C@H]7CCC(=O)NC7=O)c(F)c6)C5)CC4=O)cc23)C1 ; Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6F)CC5)CC4)cc23)C1 ; Cc1cc2cc(n1)-c1cnn(C)c1OC[C@H]1C[C@@H](CN3/C(=N/C2=O)Nc2ccc(N4CCC(N5CCN(c6c(F)cc(C7CCC(=O)NC7=O)nc6C)CC5)CC4)cc23)C1] 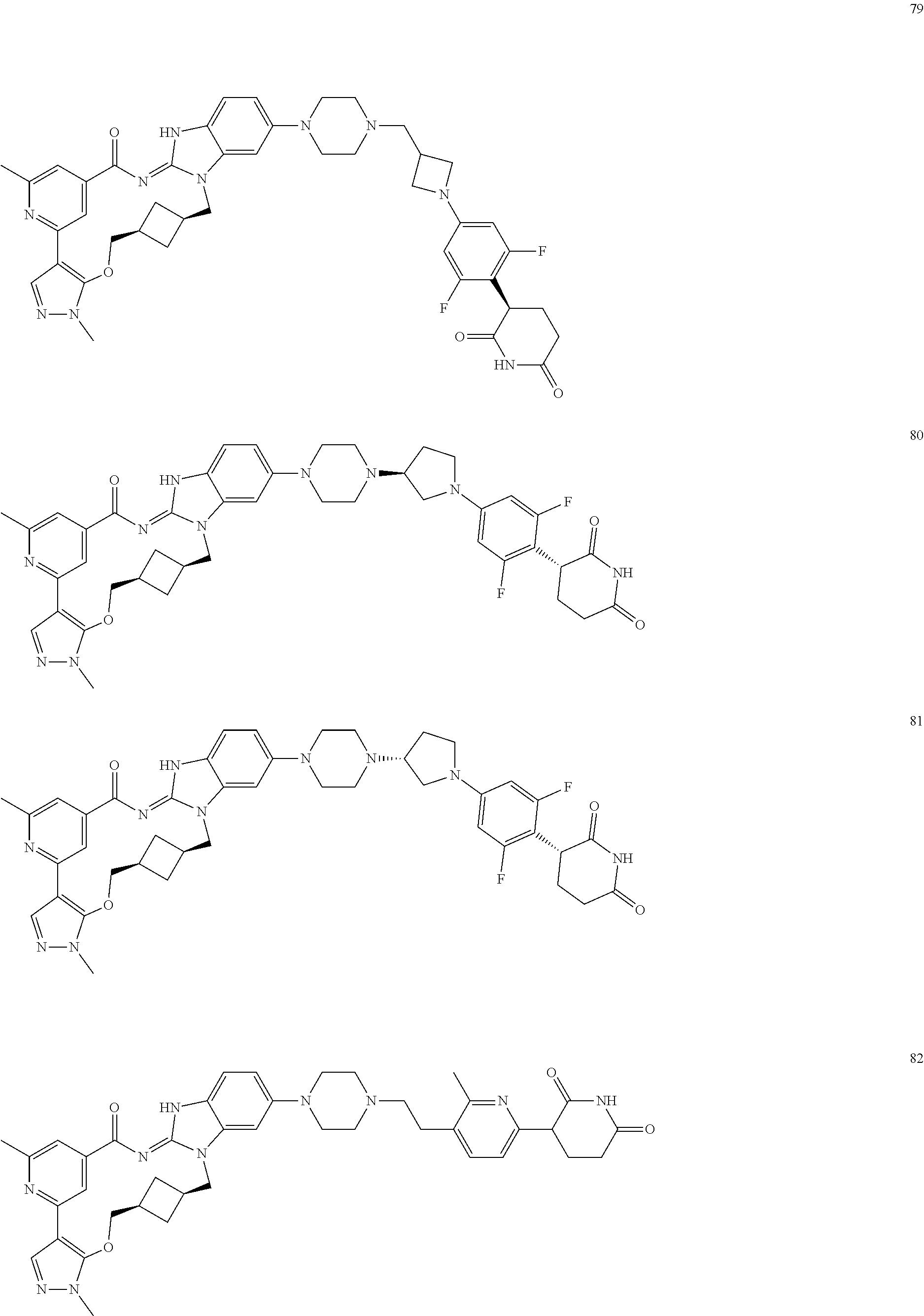 [SMILES: Cc1cc2cc(n1)-c1cnn(C)c1OC[C@H]1C[C@@H](CN3/C(=N/C2=O)Nc2ccc(N4CCN(CC5CN(c6cc(F)c([C@H]7CCC(=O)NC7=O)c(F)c6)C5)CC4)cc23)C1 ; Cc1cc2cc(n1)-c1cnn(C)c1OC[C@H]1C[C@@H](CN3/C(=N/C2=O)Nc2ccc(N4CCN([C@H]5CCN(c6cc(F)c([C@H]7CCC(=O)NC7=O)c(F)c6)C5)CC4)cc23)C1 ; Cc1cc2cc(n1)-c1cnn(C)c1OC[C@H]1C[C@@H](CN3/C(=N/C2=O)Nc2ccc(N4CCN([C@@H]5CCN(c6cc(F)c([C@H]7CCC(=O)NC7=O)c(F)c6)C5)CC4)cc23)C1 ; Cc1cc2cc(n1)-c1cnn(C)c1OC[C@H]1C[C@@H](CN3/C(=N/C2=O)Nc2ccc(N4CCN(CCc5ccc(C6CCC(=O)NC6=O)nc5C)CC4)cc23)C1] 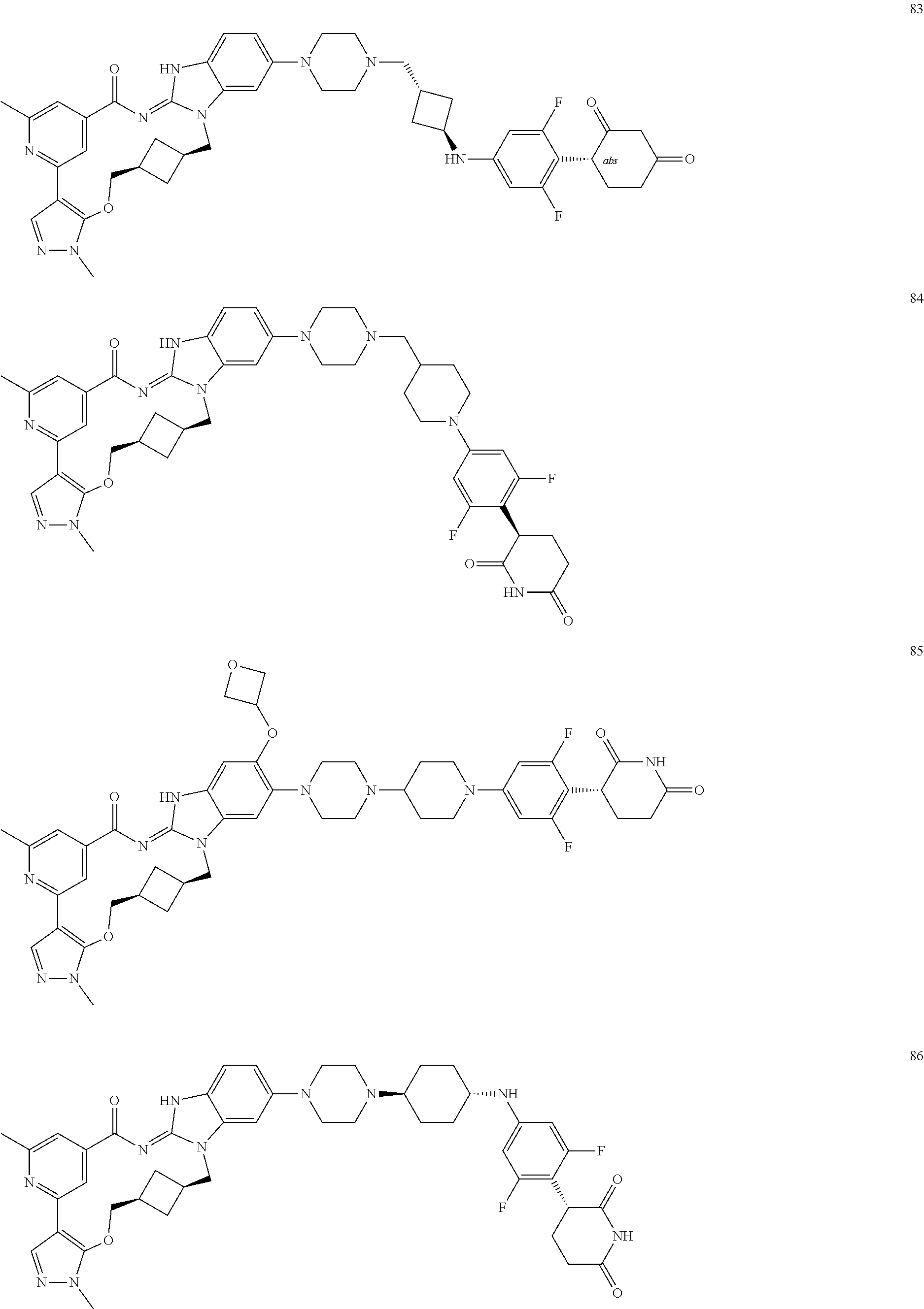 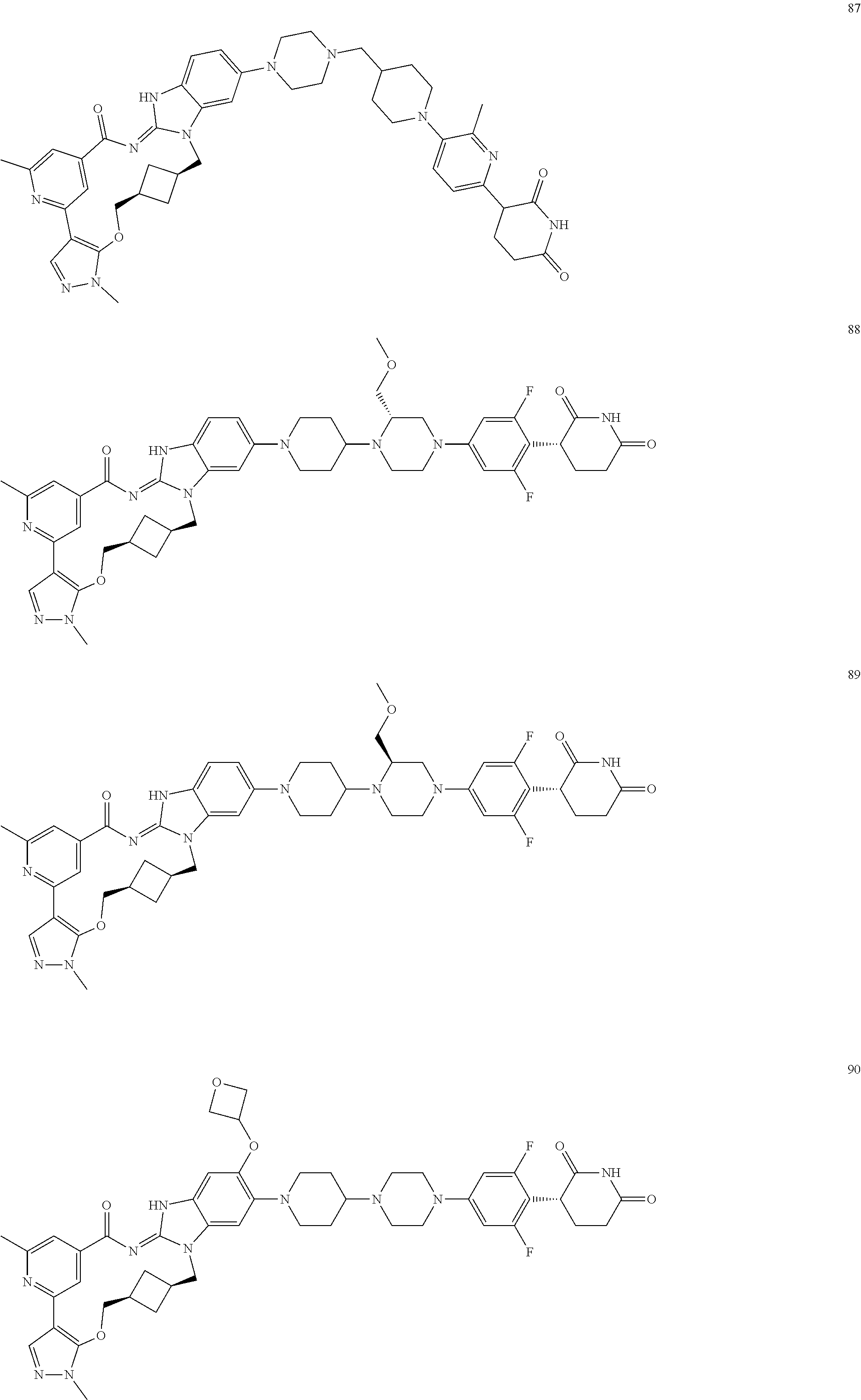 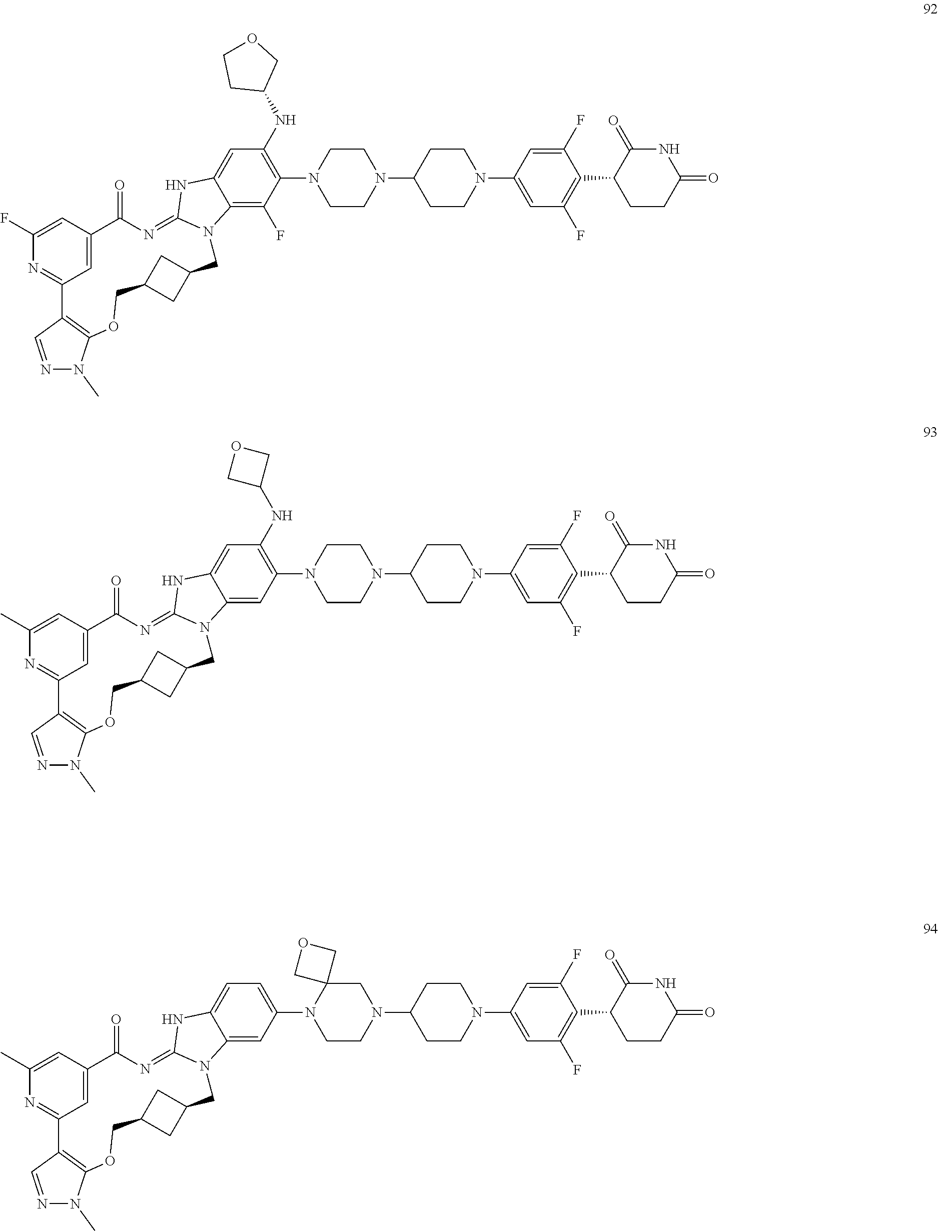 [SMILES: Cn1ncc2c1OC[C@H]1C[C@@H](CN3/C(=N/C(=O)c4cc(F)nc-2c4)Nc2cc(N[C@@H]4CCOC4)c(N4CCN(C5CCN(c6cc(F)c([C@H]7CCC(=O)NC7=O)c(F)c6)CC5)CC4)c(F)c23)C1 ; Cc1cc2cc(n1)-c1cnn(C)c1OC[C@H]1C[C@@H](CN3/C(=N/C2=O)Nc2cc(NC4COC4)c(N4CCN(C5CCN(c6cc(F)c([C@H]7CCC(=O)NC7=O)c(F)c6)CC5)CC4)cc23)C1 ; Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)CC45COC5)cc23)C1] 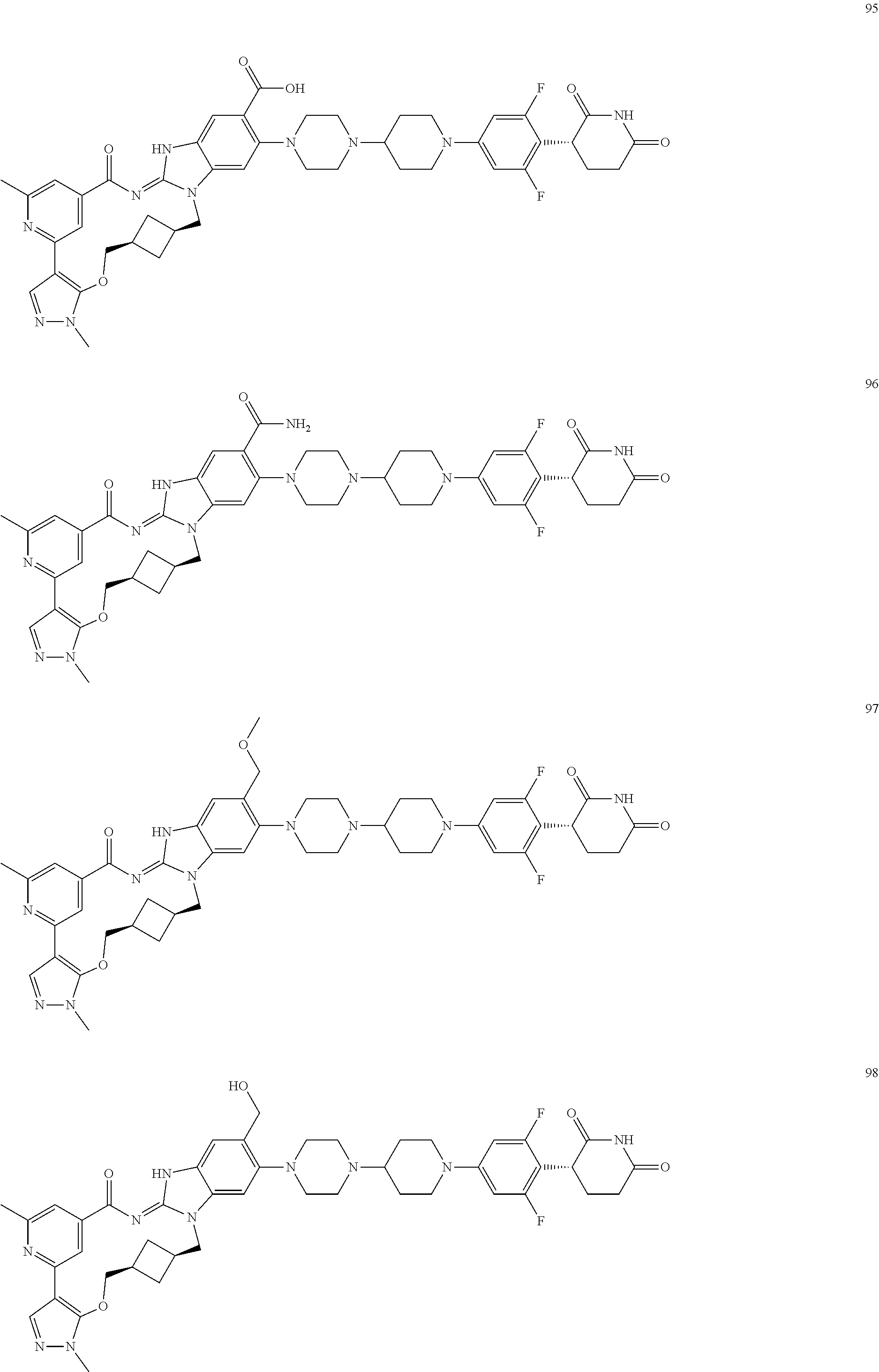 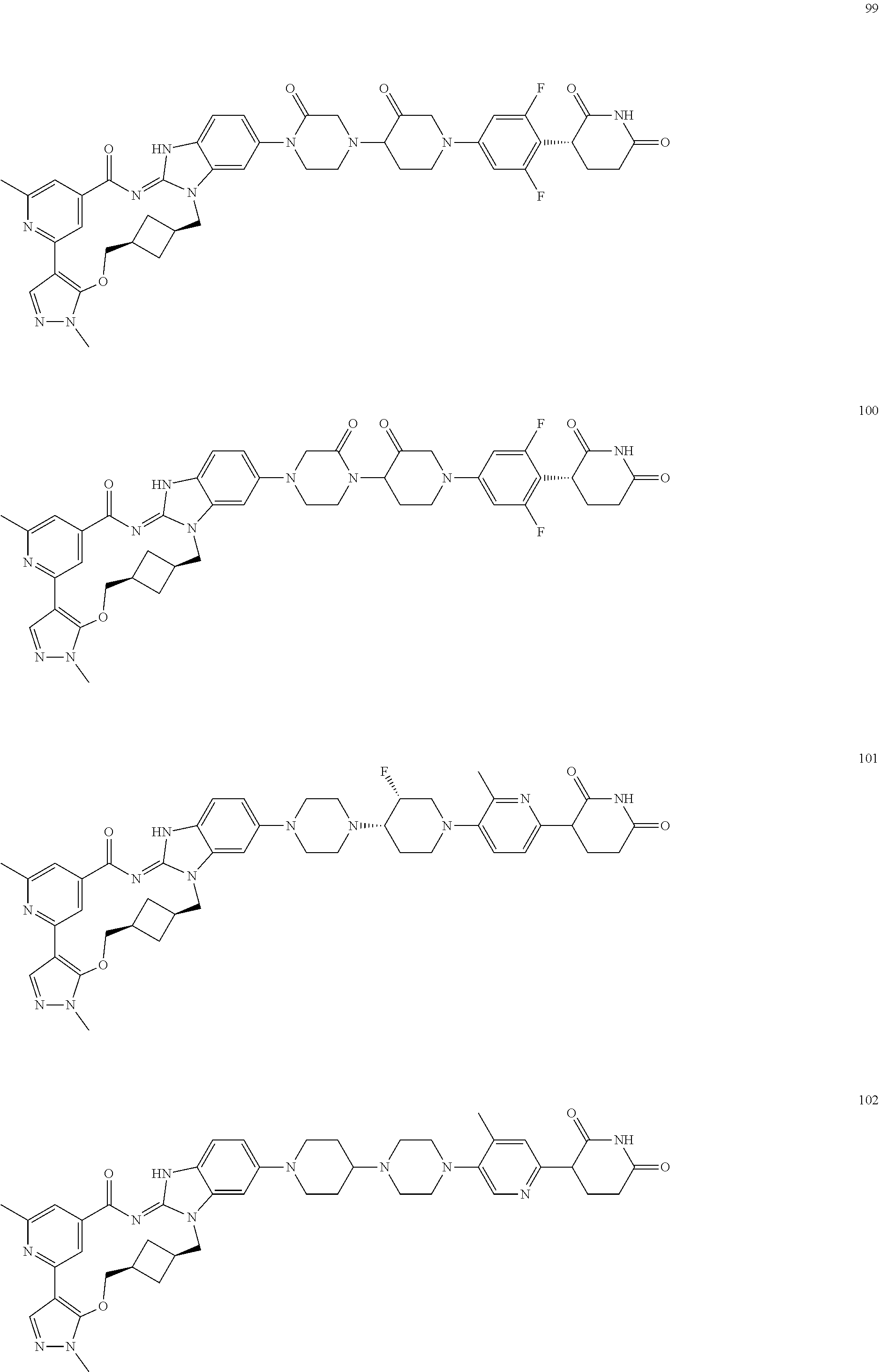 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5=O)CC4=O)cc23)C1 ; Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5=O)C(=O)C4)cc23)C1 ; Cc1cc2cc(n1)-c1cnn(C)c1OC[C@H]1C[C@@H](CN3/C(=N/C2=O)Nc2ccc(N4CCN([C@H]5CCN(c6ccc(C7CCC(=O)NC7=O)nc6C)C[C@H]5F)CC4)cc23)C1 ; Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6C)CC5)CC4)cc23)C1] 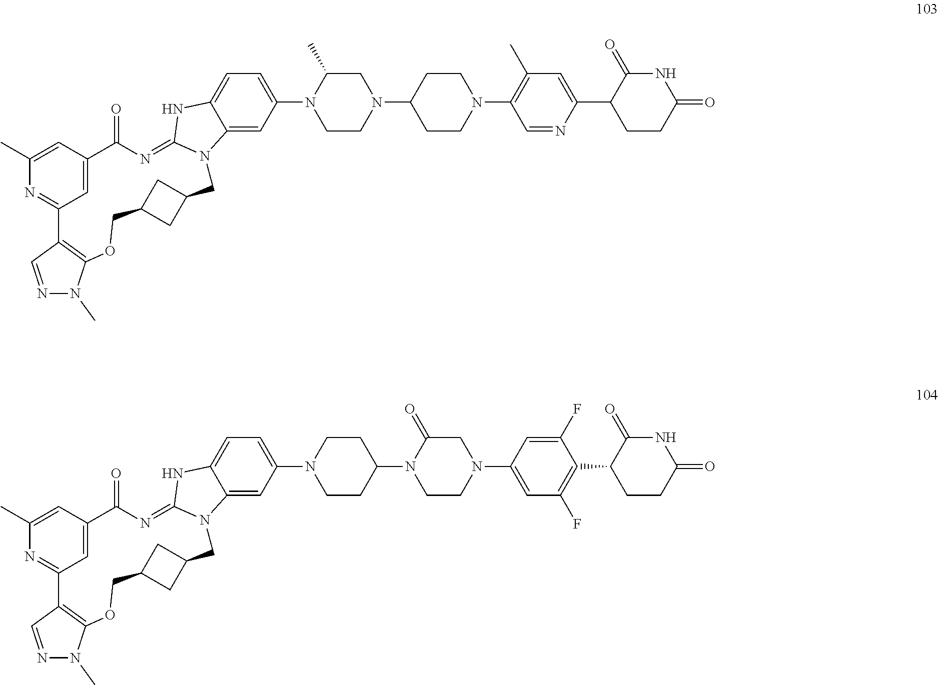 [SMILES: Cc1cc2cc(n1)-c1cnn(C)c1OC[C@H]1C[C@@H](CN3/C(=N/C2=O)Nc2ccc(N4CCN(C5CCN(c6cnc(C7CCC(=O)NC7=O)cc6C)CC5)C[C@H]4C)cc23)C1 ; Cc1cc2cc(n1)-c1cnn(C)c1OC[C@H]1C[C@@H](CN3/C(=N/C2=O)Nc2ccc(N4CCC(N5CCN(c6cc(F)c([C@H]7CCC(=O)NC7=O)c(F)c6)CC5=O)CC4)cc23)C1] 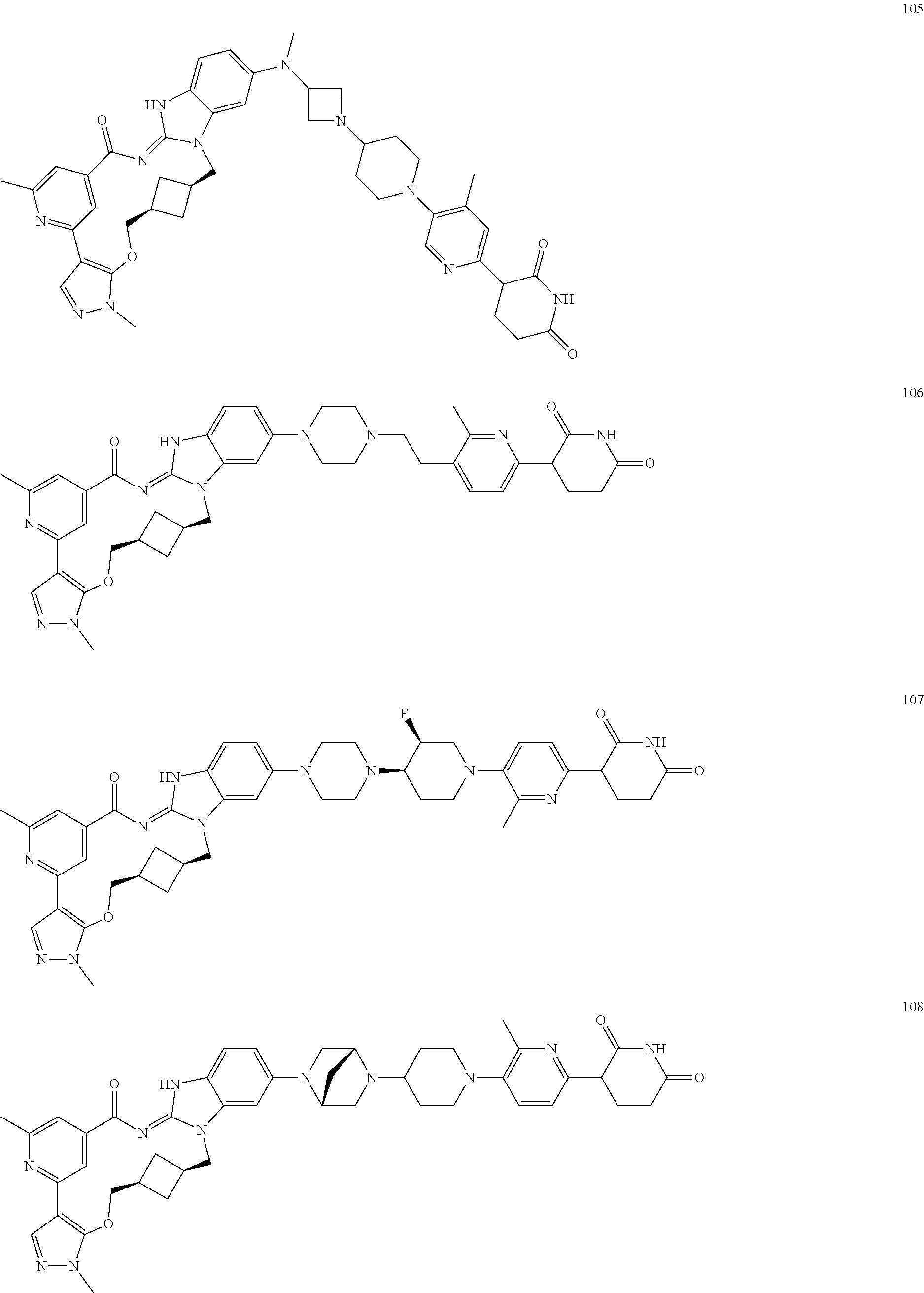 [SMILES: Cc1cc2cc(n1)-c1cnn(C)c1OC[C@H]1C[C@@H](CN3/C(=N/C2=O)Nc2ccc(N(C)C4CN(C5CCN(c6cnc(C7CCC(=O)NC7=O)cc6C)CC5)C4)cc23)C1 ; Cc1cc2cc(n1)-c1cnn(C)c1OC[C@H]1C[C@@H](CN3/C(=N/C2=O)Nc2ccc(N4CCN(CCc5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)C[C@@H]5F)CC4)cc23)C1 ; Cc1cc2cc(n1)-c1cnn(C)c1OC[C@H]1C[C@@H](CN3/C(=N/C2=O)Nc2ccc(N4C[C@H]5C[C@@H]4CN5C4CCN(c5ccc(C6CCC(=O)NC6=O)nc5C)CC4)cc23)C1] 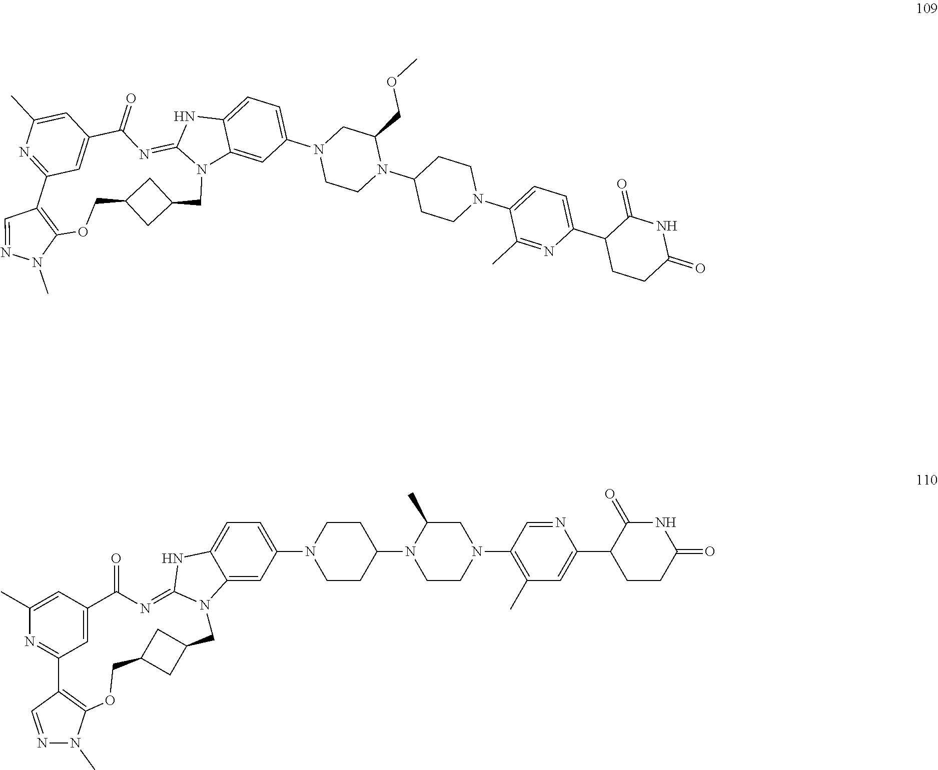 [SMILES: COC[C@@H]1CN(c2ccc3c(c2)N2C[C@H]4C[C@@H](COc5c(cnn5C)-c5cc(cc(C)n5)C(=O)/N=C/2N3)C4)CCN1C1CCN(c2ccc(C3CCC(=O)NC3=O)nc2C)CC1 ; Cc1cc2cc(n1)-c1cnn(C)c1OC[C@H]1C[C@@H](CN3/C(=N/C2=O)Nc2ccc(N4CCC(N5CCN(c6cnc(C7CCC(=O)NC7=O)cc6C)C[C@@H]5C)CC4)cc23)C1] 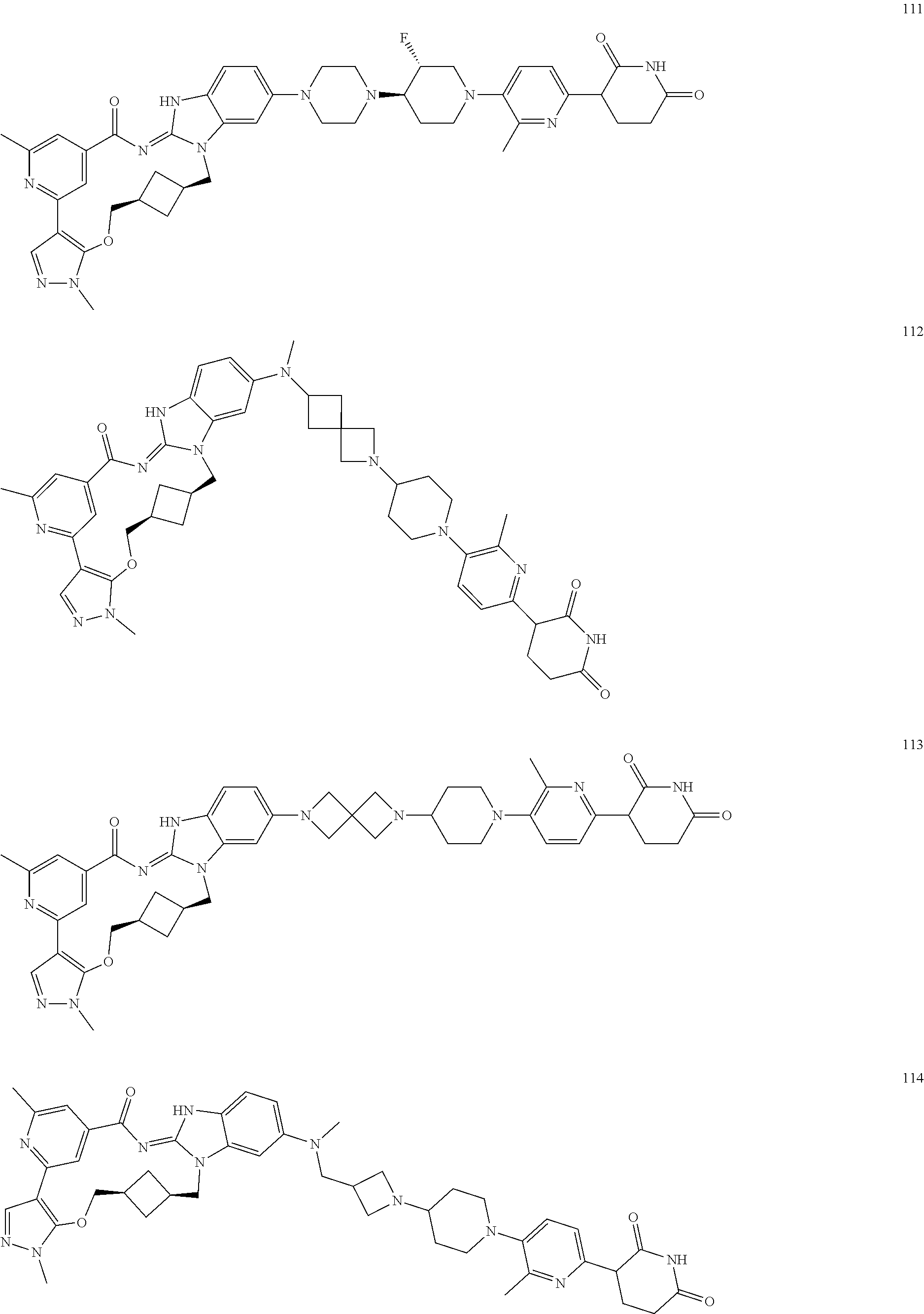 [SMILES: Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)C[C@H]5F)CC4)cc23)C1 ; Cc1cc2cc(n1)-c1cnn(C)c1OC[C@H]1C[C@@H](CN3/C(=N/C2=O)Nc2ccc(N(C)C4CC5(C4)CN(C4CCN(c6ccc(C7CCC(=O)NC7=O)nc6C)CC4)C5)cc23)C1 ; Cc1cc2cc(n1)-c1cnn(C)c1OC[C@H]1C[C@@H](CN3/C(=N/C2=O)Nc2ccc(N4CC5(C4)CN(C4CCN(c6ccc(C7CCC(=O)NC7=O)nc6C)CC4)C5)cc23)C1 ; Cc1cc2cc(n1)-c1cnn(C)c1OC[C@H]1C[C@@H](CN3/C(=N/C2=O)Nc2ccc(N(C)CC4CN(C5CCN(c6ccc(C7CCC(=O)NC7=O)nc6C)CC5)C4)cc23)C1] 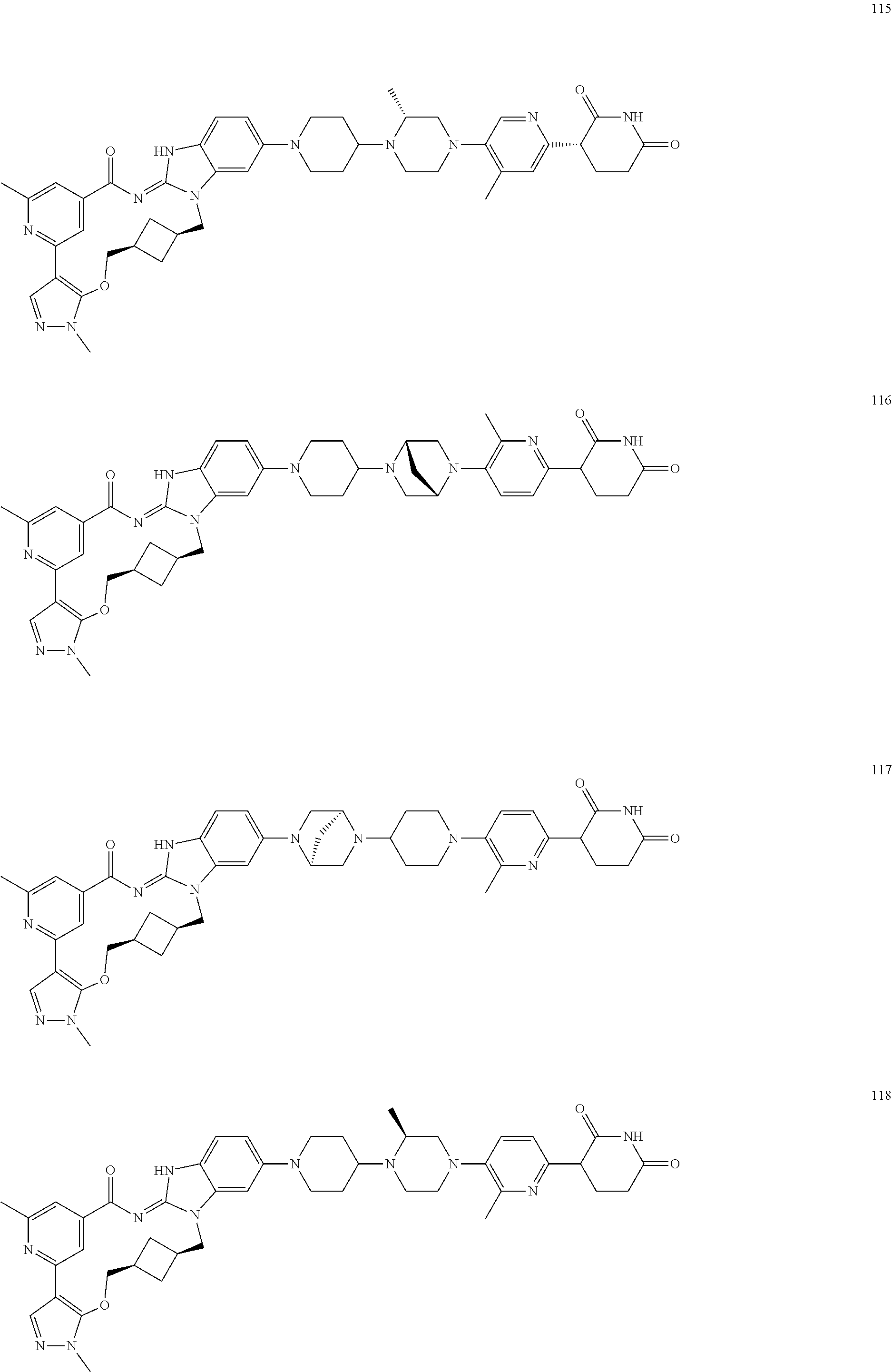 [SMILES: Cc1cc2cc(n1)-c1cnn(C)c1OC[C@H]1C[C@@H](CN3/C(=N/C2=O)Nc2ccc(N4CCC(N5CCN(c6cnc([C@H]7CCC(=O)NC7=O)cc6C)C[C@H]5C)CC4)cc23)C1 ; Cc1cc2cc(n1)-c1cnn(C)c1OC[C@H]1C[C@@H](CN3/C(=N/C2=O)Nc2ccc(N4CCC(N5C[C@@H]6C[C@H]5CN6c5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4C[C@@H]5C[C@H]4CN5C4CCN(c5ccc(C6CCC(=O)NC6=O)nc5C)CC4)cc23)C1 ; Cc1cc2cc(n1)-c1cnn(C)c1OC[C@H]1C[C@@H](CN3/C(=N/C2=O)Nc2ccc(N4CCC(N5CCN(c6ccc(C7CCC(=O)NC7=O)nc6C)C[C@@H]5C)CC4)cc23)C1] 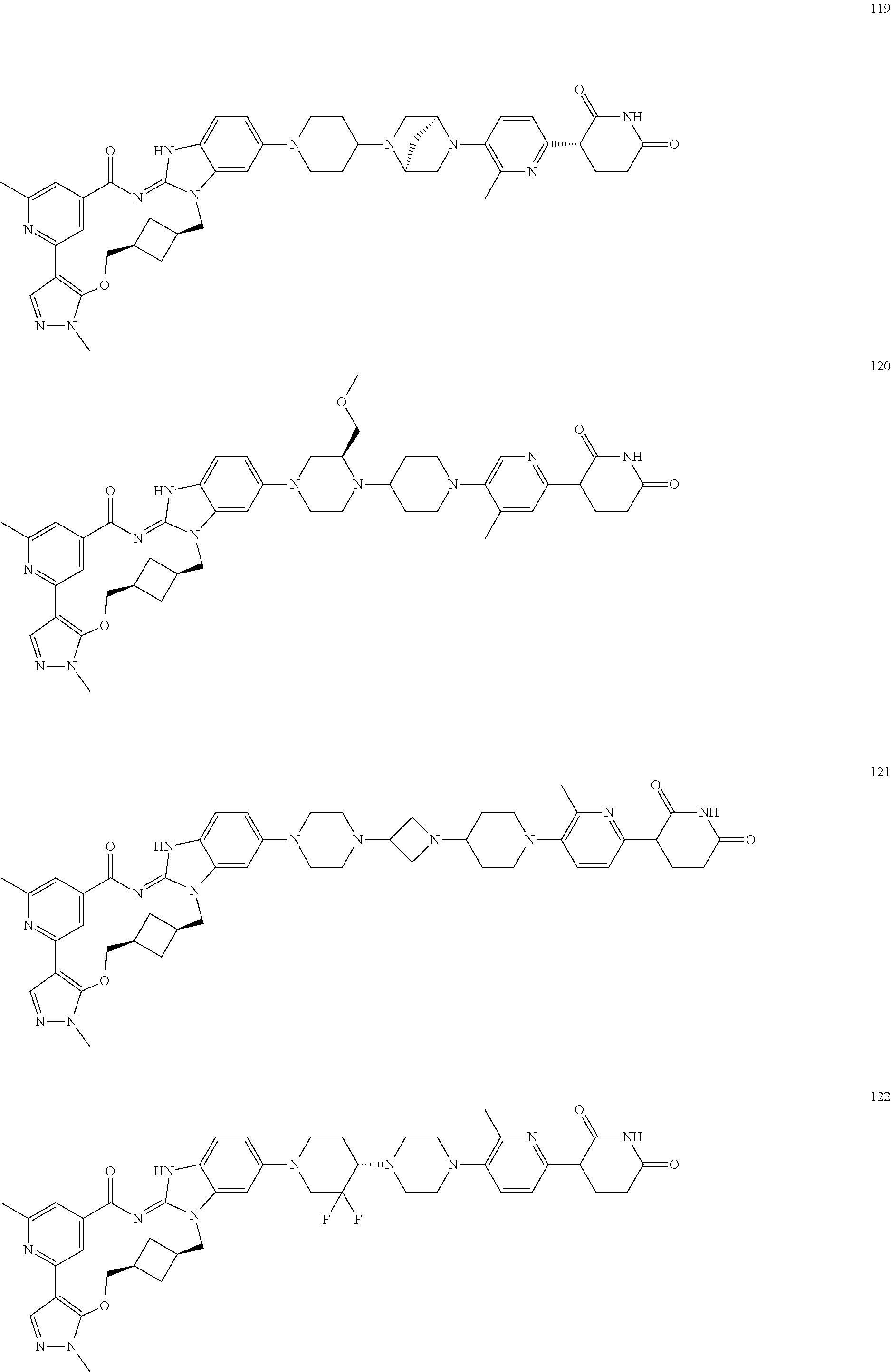 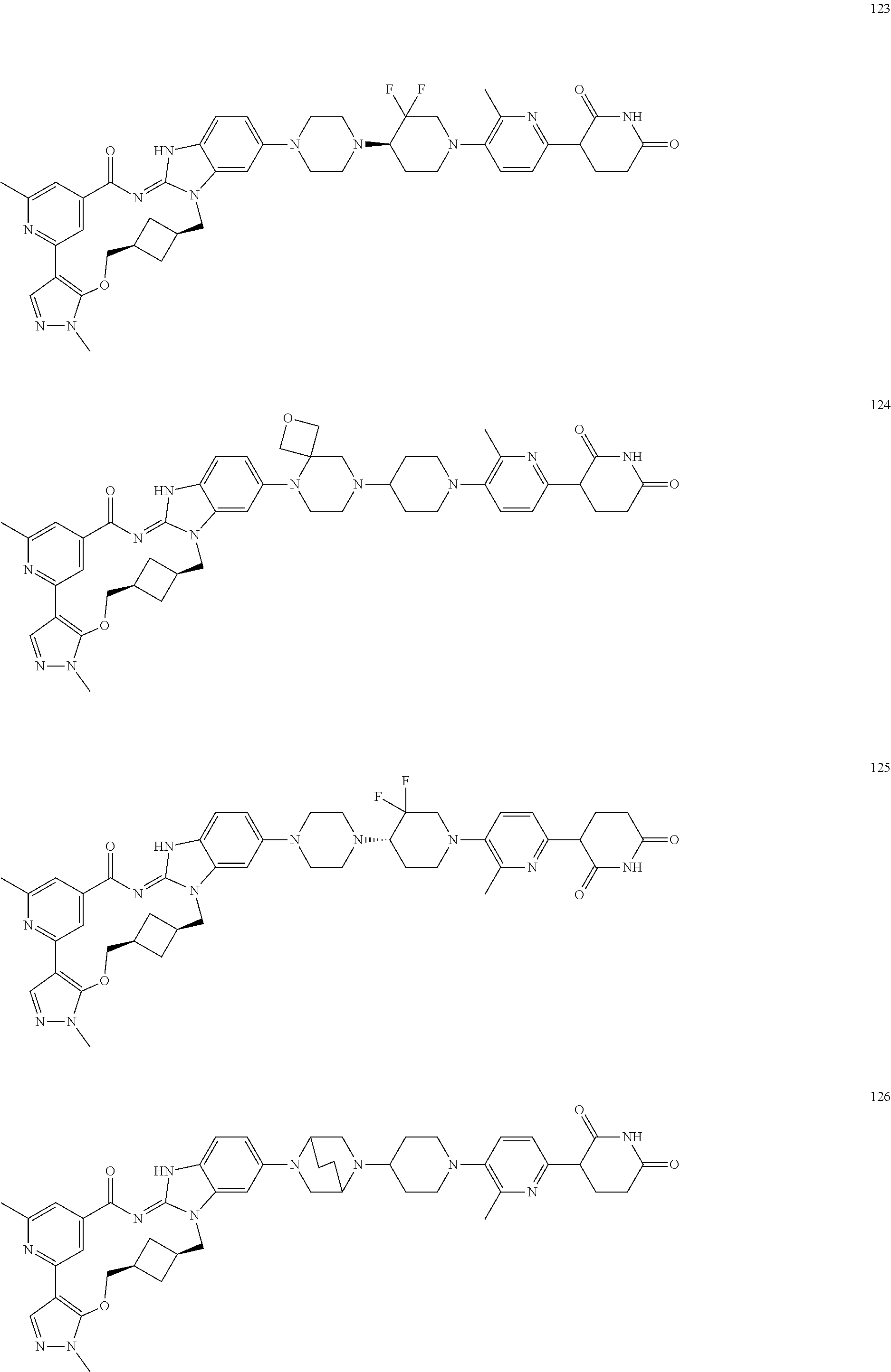 [SMILES: Cc1cc2cc(n1)-c1cnn(C)c1OC[C@H]1C[C@@H](CN3/C(=N/C2=O)Nc2ccc(N4CCN([C@@H]5CCN(c6ccc(C7CCC(=O)NC7=O)nc6C)CC5(F)F)CC4)cc23)C1 ; Cc1cc2cc(n1)-c1cnn(C)c1OC[C@H]1C[C@@H](CN3/C(=N/C2=O)Nc2ccc(N4CCN(C5CCN(c6ccc(C7CCC(=O)NC7=O)nc6C)CC5)CC45COC5)cc23)C1 ; Cc1cc2cc(n1)-c1cnn(C)c1OC[C@H]1C[C@@H](CN3/C(=N/C2=O)Nc2ccc(N4CCN([C@H]5CCN(c6ccc(C7CCC(=O)NC7=O)nc6C)CC5(F)F)CC4)cc23)C1 ; Cc1cc2cc(n1)-c1cnn(C)c1OC[C@H]1C[C@@H](CN3/C(=N/C2=O)Nc2ccc(N4CC5CCC4CN5C4CCN(c5ccc(C6CCC(=O)NC6=O)nc5C)CC4)cc23)C1] 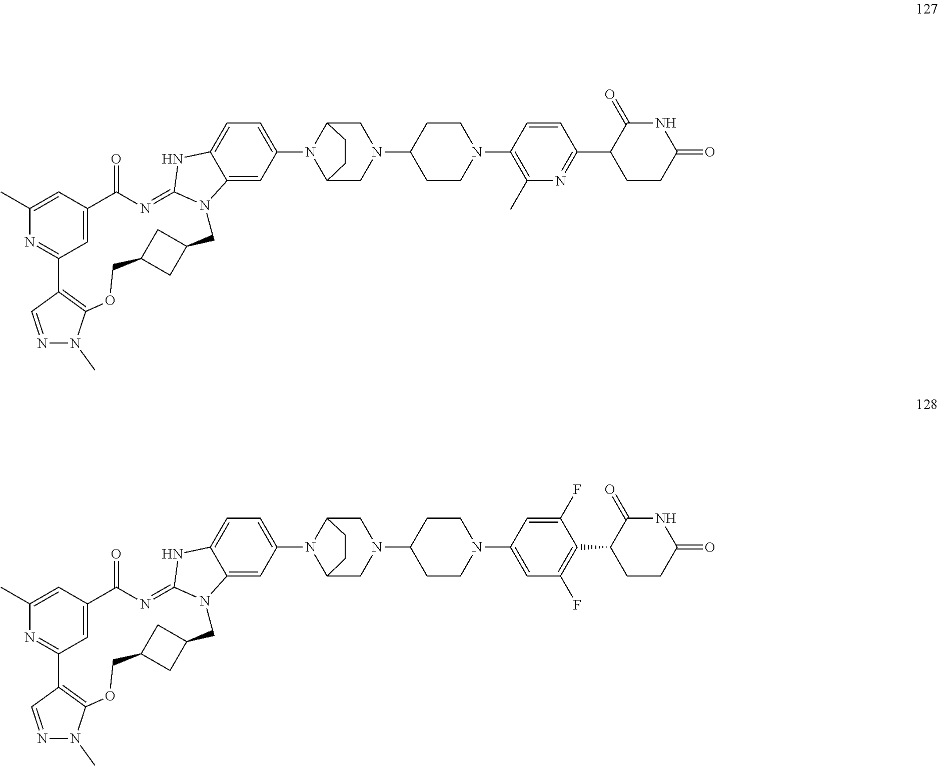 [SMILES: Cc1cc2cc(n1)-c1cnn(C)c1OC[C@H]1C[C@@H](CN3/C(=N/C2=O)Nc2ccc(N4C5CCC4CN(C4CCN(c6ccc(C7CCC(=O)NC7=O)nc6C)CC4)C5)cc23)C1 ; Cc1cc2cc(n1)-c1cnn(C)c1OC[C@H]1C[C@@H](CN3/C(=N/C2=O)Nc2ccc(N4C5CCC4CN(C4CCN(c6cc(F)c([C@H]7CCC(=O)NC7=O)c(F)c6)CC4)C5)cc23)C1] 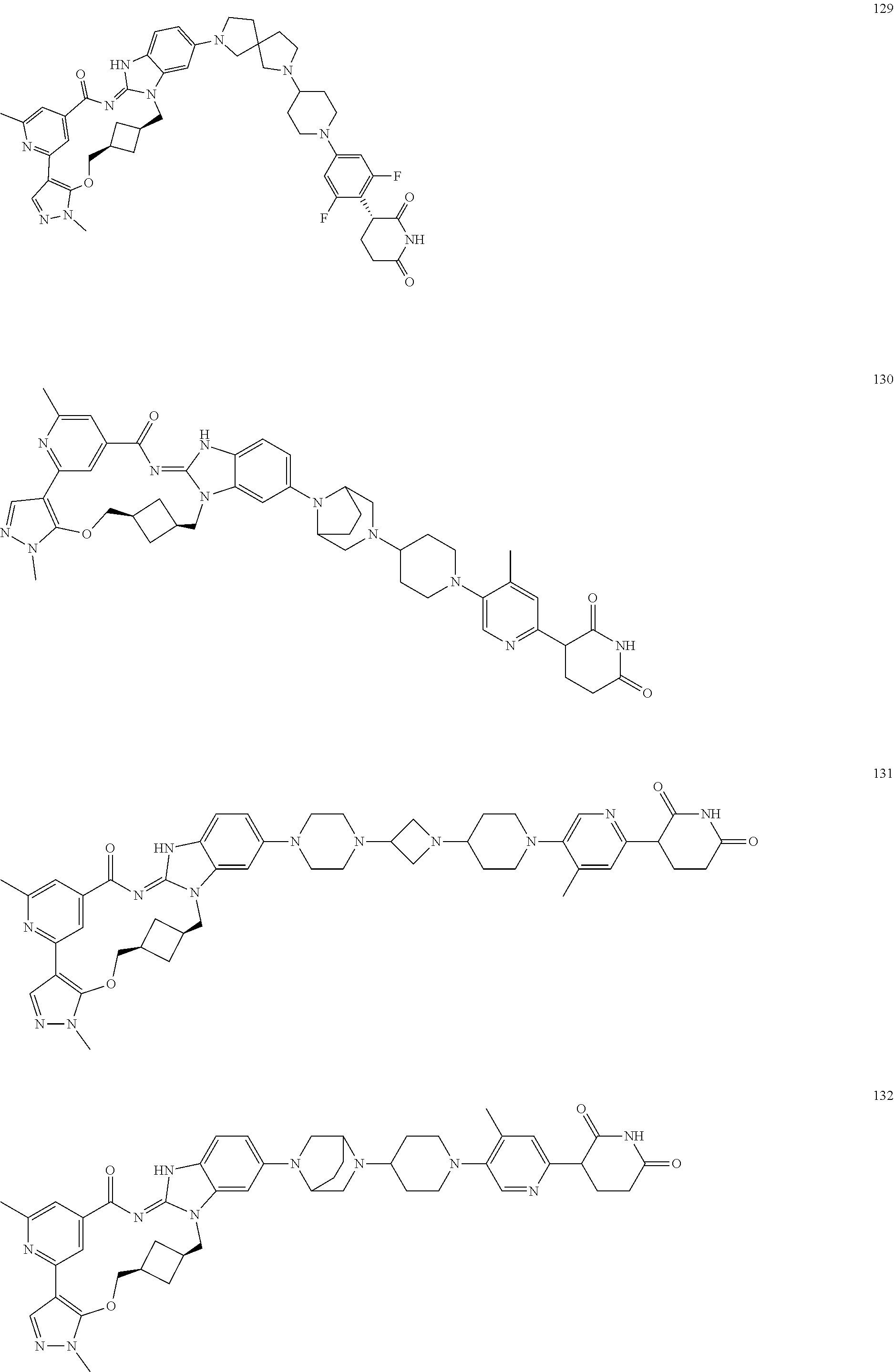 [SMILES: Cc1cc2cc(n1)-c1cnn(C)c1OC[C@H]1C[C@@H](CN3/C(=N/C2=O)Nc2ccc(N4CCC5(CCN(C6CCN(c7cc(F)c([C@H]8CCC(=O)NC8=O)c(F)c7)CC6)C5)C4)cc23)C1 ; Cc1cc2cc(n1)-c1cnn(C)c1OC[C@H]1C[C@@H](CN3/C(=N/C2=O)Nc2ccc(N4C5CCC4CN(C4CCN(c6cnc(C7CCC(=O)NC7=O)cc6C)CC4)C5)cc23)C1 ; Cc1cc2cc(n1)-c1cnn(C)c1OC[C@H]1C[C@@H](CN3/C(=N/C2=O)Nc2ccc(N4CCN(C5CN(C6CCN(c7cnc(C8CCC(=O)NC8=O)cc7C)CC6)C5)CC4)cc23)C1 ; Cc1cc2cc(n1)-c1cnn(C)c1OC[C@H]1C[C@@H](CN3/C(=N/C2=O)Nc2ccc(N4CC5CCC4CN5C4CCN(c5cnc(C6CCC(=O)NC6=O)cc5C)CC4)cc23)C1] 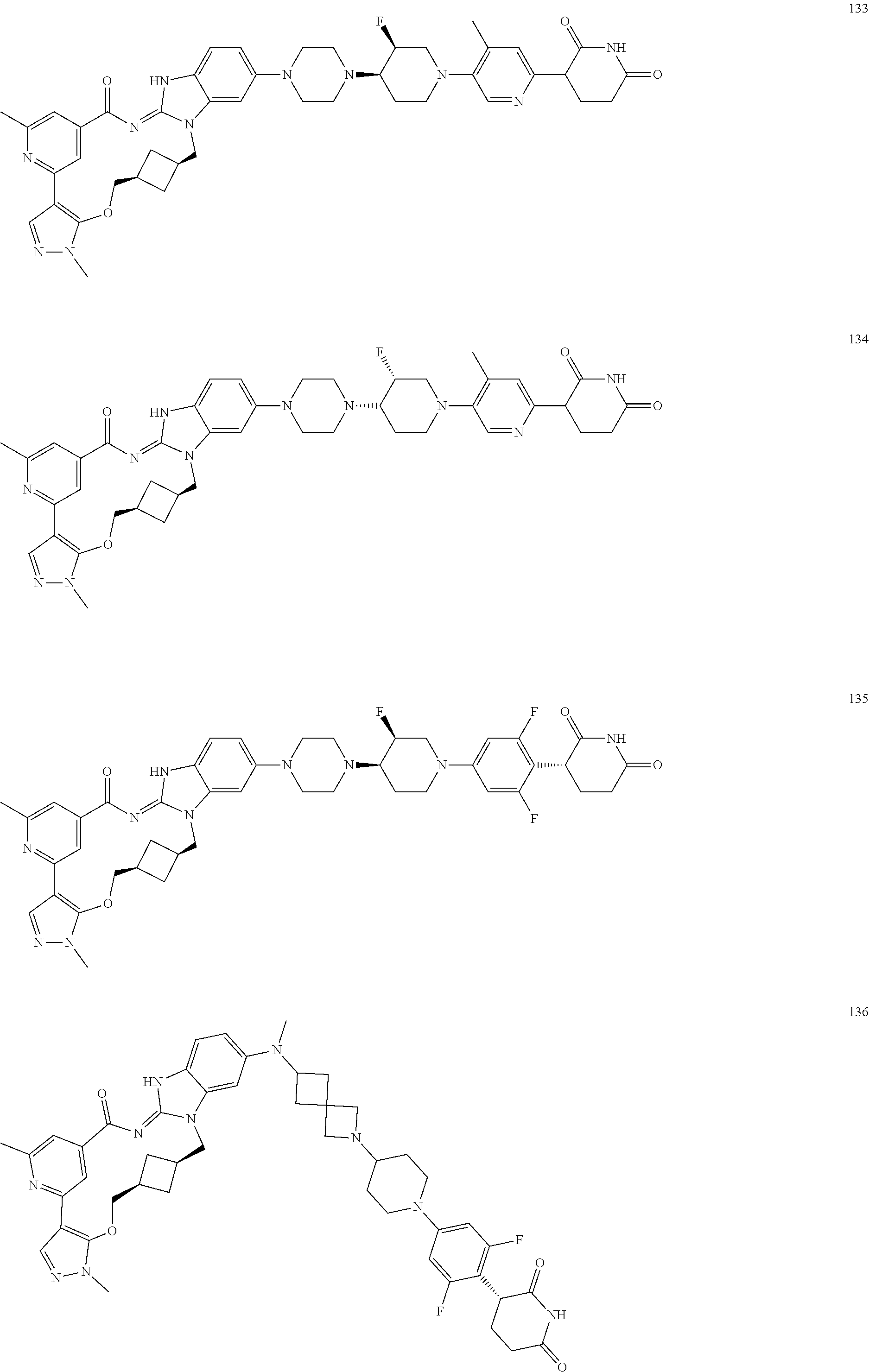 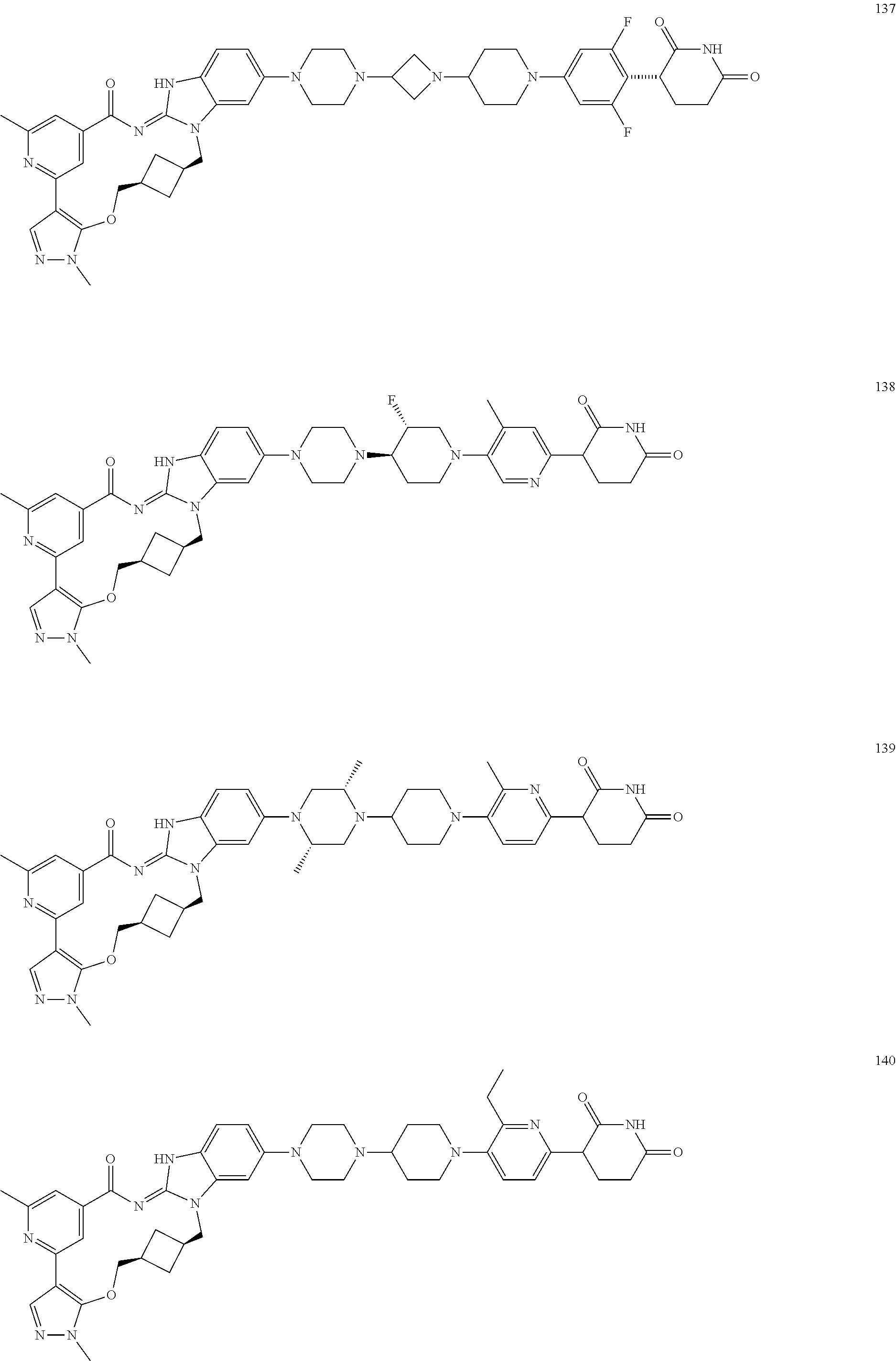 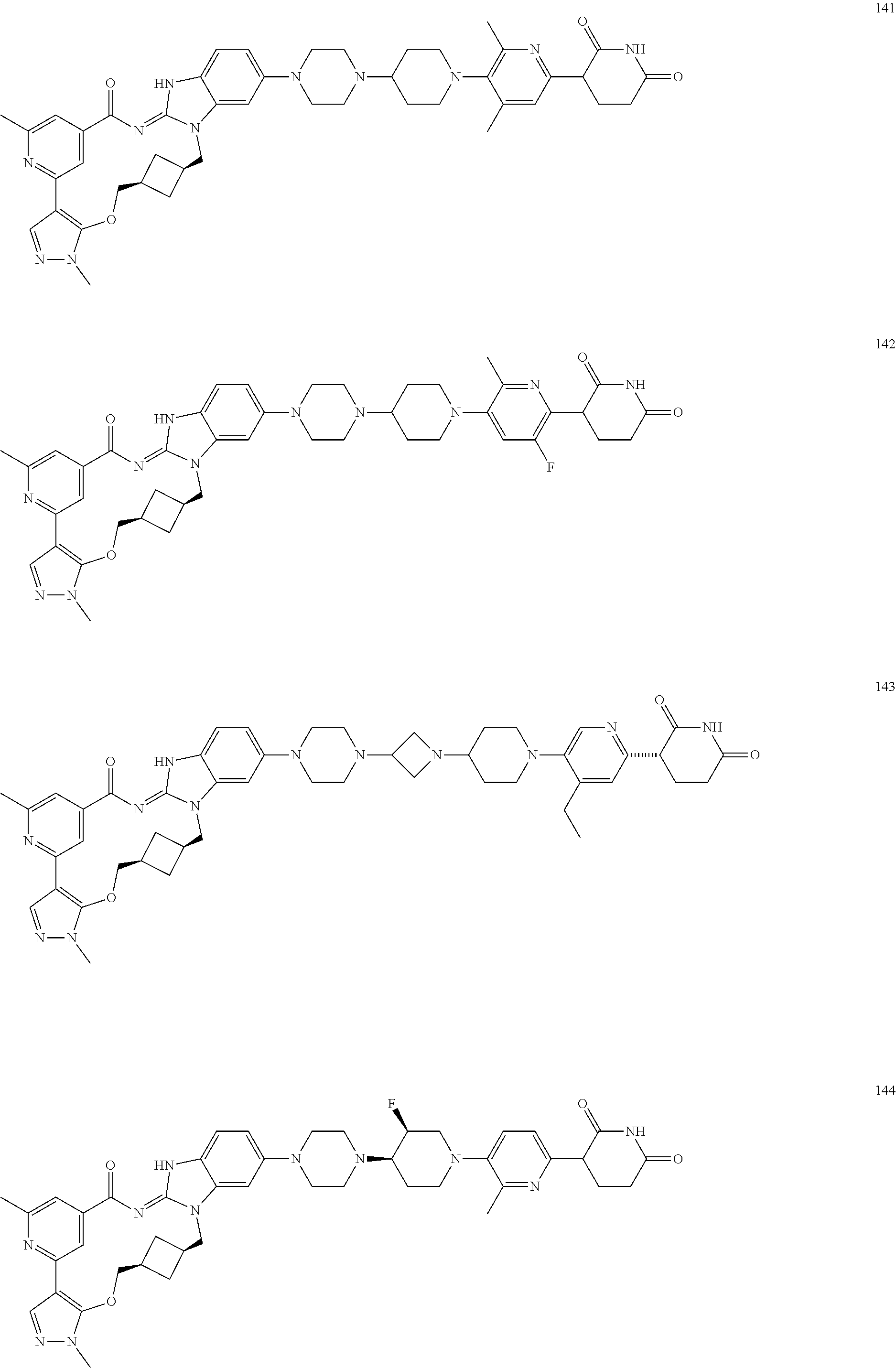 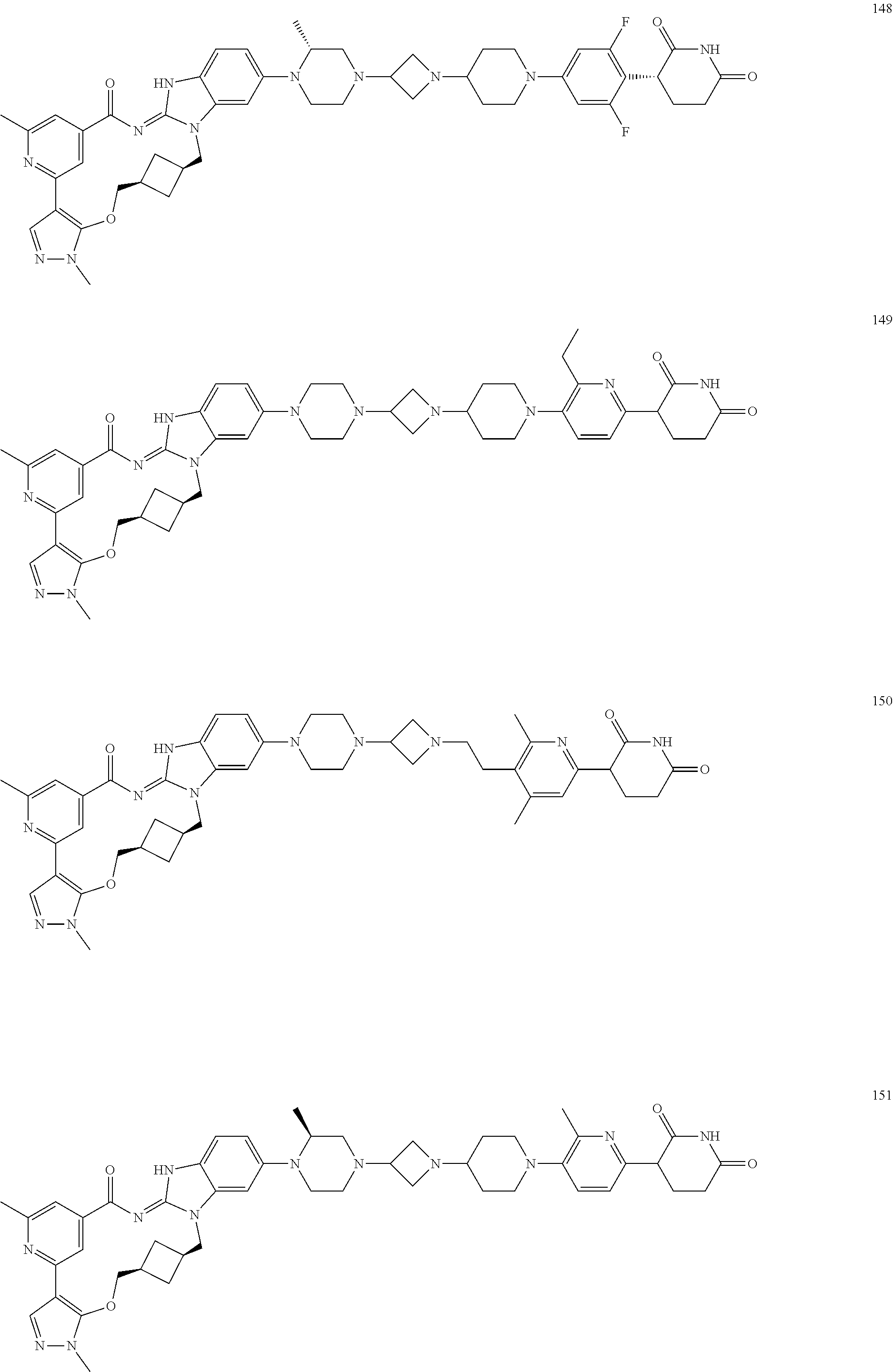 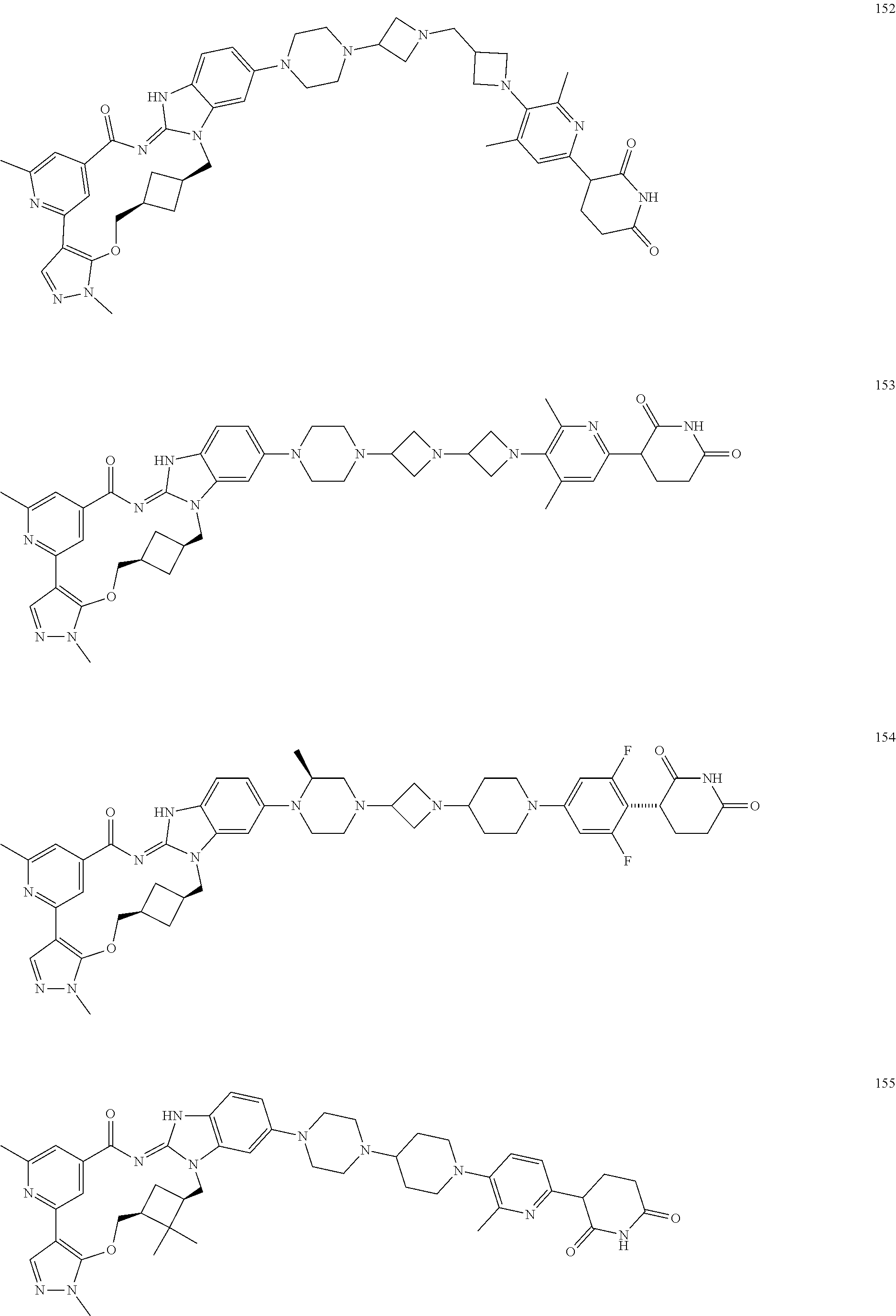 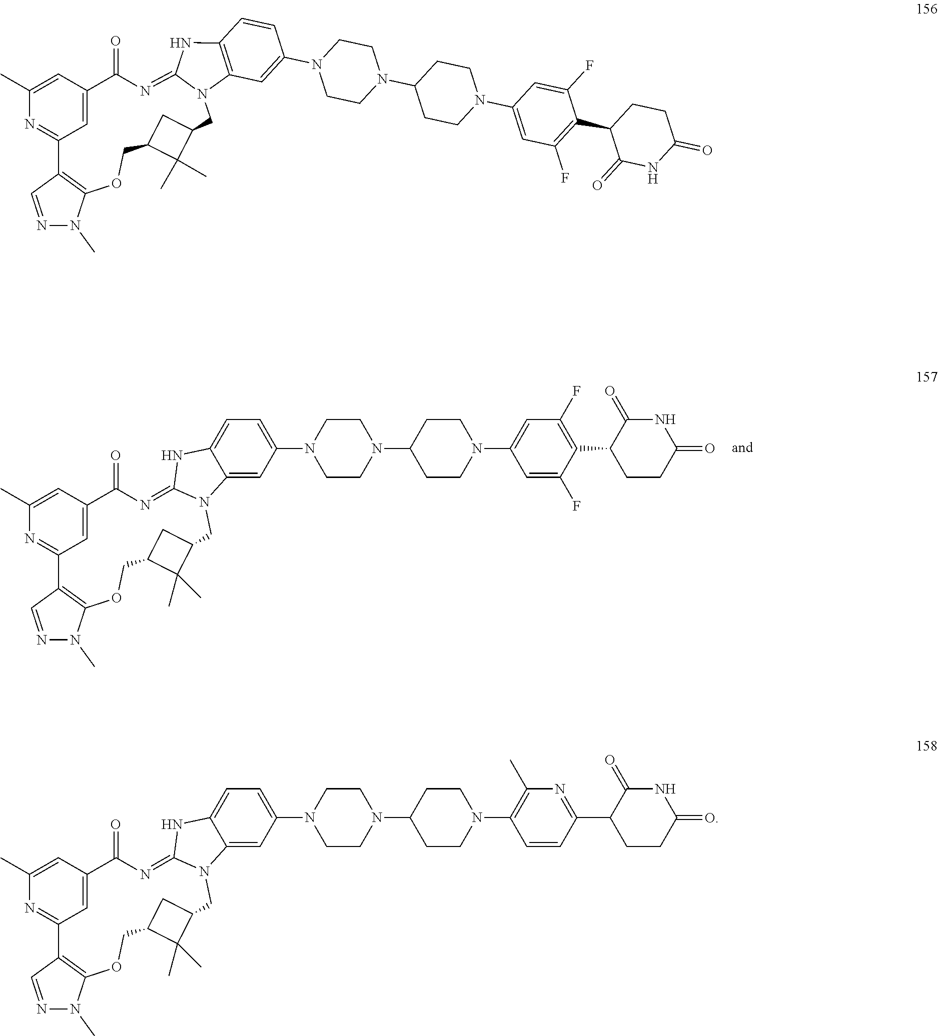 [SMILES: Cc1cc2cc(n1)-c1cnn(C)c1OC[C@H]1C[C@@H](CN3/C(=N/C2=O)Nc2ccc(N4CCN(C5CCN(c6cc(F)c([C@H]7CCC(=O)NC7=O)c(F)c6)CC5)CC4)cc23)C1(C)C ; Cc1cc2cc(n1)-c1cnn(C)c1OC[C@@H]1C[C@H](CN3/C(=N/C2=O)Nc2ccc(N4CCN(C5CCN(c6cc(F)c([C@H]7CCC(=O)NC7=O)c(F)c6)CC5)CC4)cc23)C1(C)C ; Cc1cc2cc(n1)-c1cnn(C)c1OC[C@@H]1C[C@H](CN3/C(=N/C2=O)Nc2ccc(N4CCN(C5CCN(c6ccc(C7CCC(=O)NC7=O)nc6C)CC5)CC4)cc23)C1(C)C]
or a pharmaceutically acceptable salt thereof or a stereoisomer thereof, or a tautomer thereof, or a deuterated analog thereof.

36. A pharmaceutical composition comprising a compound of claim 1, or a pharmaceutically acceptable salt, stereoisomer, tautomer or prodrug thereof, together with a pharmaceutically acceptable excipient.

37. A method of treating a disease that can be affected by EGFR modulation, comprises administrating a subject in need thereof an effective amount of a compound of claim 1, or a pharmaceutically acceptable salt, stereoisomer, tautomer or prodrug thereof, wherein the disease is selected from cancer, pancreatic cancer, breast cancer, glioblastoma multiforme, head and neck cancer, or non-small cell lung cancer.

38.-40. (canceled)

41. The compound of claim 1, wherein R¹⁰ is each independently selected from hydrogen, —F, —Cl, —Br, —I, methyl, ethyl, propyl, butyl, pentyl, hexyl, cyclopropyl, cyclobutyl, cyclopentyl, cyclohexyl, 3- to 8-membered heterocyclyl, —NR^{10a}R^{10b}, —OR^{10a}, —CO₂R^{10a} or —C(O)NR^{10a}R^{10b}; each of methyl, ethyl, propyl, butyl, pentyl, hexyl, cyclopropyl, cyclobutyl, cyclopenyl, cyclohexyl or 3- to 8-membered heterocyclyl is optionally substituted with at least one R^{10c};
R^{10a} and R^{10b} are each independently selected from hydrogen, methyl, ethyl, propyl, butyl, pentyl, cyclopropyl, cyclobutyl, cyclopentyl, cyclohexyl, 3-membered heterocyclyl, 4-membered heterocyclyl, 5-membered heterocyclyl or 6-membered lieterocyclyl;
R^{10c} is each independently selected from hydrogen, —F, —Cl, —Br, —I, methyl, ethyl, propyl, butyl, cyclopropyl, cyclobutyl, cyclopentyl, oxo (═O), —NR^{10e}R^{10f}, —OR^{10e} or —CN;
R^{10e} and R^{10f} are each independently selected from hydrogen, methyl, ethyl, propyl, cyclopropyl, cyclobutyl, cyclopentyl;

42. The compound of claim 1, wherein R¹⁰ is each independently selected from H, F, Cl, Br, methyl, ethyl, propyl (n-propyl or iso-propyl), butyl(n-butyl, sec-butyl, iso-butyl or tert-butyl), cyclopropyl, cyclobutyl, cyclopentyl, cyclohexyl, 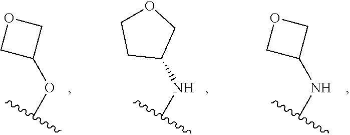 [SMILES: *OC1COC1 ; *N[C@@H]1CCOC1 ; *NC1COC1]
—COOH, —CONH₂, —C—OCH or —CH₂OH.
